(19) United States
(12) Patent Application Publication
(10) Pub. No.: US 2025/0206724 A1
(43) Pub. Date: Jun. 26, 2025

(54) SUBSTITUTED 3-AMINO INDAZOLE DERIVATIVES AS KINASE INHIBITORS

(71) Applicant: NERVIANO MEDICAL SCIENCES S.R.L., Nerviano (MI) (IT)

(72) Inventors: Paolo POLUCCI, Cassina Rizzardi (CO) (IT); Maria MENICHINCHERI, Milan (IT); Ilaria MOTTO, Nerviano (IT); Matteo SALSA, Bellinzago Novarese (NO) (IT); Matteo D'ANELLO, Novate Milanese (MI) (IT); Daniela FAIARDI, Pavia (IT)

(73) Assignee: NERVIANO MEDICAL SCIENCES S.R.L., Nerviano (MI) (IT)

(21) Appl. No.: 18/852,095

(22) PCT Filed: Mar. 27, 2023
(86) PCT No.: PCT/EP2023/057772

§ 371 Date: Sep. 27, 2024

(30) Foreign Application Priority Data
Mar. 30, 2022 (EP) 22165550.9

(51) Int. Cl. C07D 403/12; A61K 31/416; A61K 31/438; A61K 31/439; A61K 31/454; A61K 31/5377; A61K 31/55; A61K 45/06; A61P 35/02; C07D 231/56; C07D 401/12; C07D 401/14; C07D 405/12; C07D 405/14; C07D 413/12; C07D 413/14; C07D 471/08

(52) U.S. Cl. CPC C07D 403/12; A61K 31/416; A61K 31/438; A61K 31/439; A61K 31/454; A61K 31/5377; A61K 31/55; A61K 45/06; A61P 35/02; C07D 231/56; C07D 401/12; C07D 401/14; C07D 405/12; C07D 405/14; C07D 413/12; C07D 413/14; C07D 471/08

(57) ABSTRACT

The present invention relates to substituted 3-amino indazole derivatives as kinase inhibitors and in particular as inhibitors of CDK11 families. Due to the key role of protein kinases, in particular of CDK11, in the regulation of cellular proliferation, such compounds are thus useful to treat diseases caused by altered CDK11 activity, in particular in the treatment of cancer as well as in the treatment of a variety of cell proliferative disorders and immune-related disorder. The present invention also provides methods for preparing these compounds, pharmaceutical compositions comprising these compounds, and methods of treating diseases utilizing pharmaceutical compositions comprising these compounds.

The present invention relates to substituted 3-amino indazole derivatives, to a process for their preparation, to pharmaceutical compositions comprising them, and to their use as therapeutic agents, particularly in the treatment of diseases caused by dysregulated protein kinase activity, such as cancer, cell proliferative disorders, viral infections, immune disorders, neurodegenerative disorders and cardiovascular diseases.

### BACKGROUND OF THE INVENTION

The malfunctioning of protein kinases (PKs) is the hallmark of numerous diseases. A large share of the oncogenes and proto-oncogenes involved in human cancers encode for PKs. The enhanced activities of PKs are also implicated in many non-malignant diseases, such as benign prostate hyperplasia, familial adenomatosis, polyposis, neurofibromatosis, psoriasis, vascular smooth cell proliferation associated with atherosclerosis, pulmonary fibrosis, arthritis glomerulonephritis and post-surgical stenosis and restenosis.

PKs are also implicated in inflammatory conditions and in the multiplication of viruses and parasites. PKs may also play a major role in the pathogenesis and development of neurodegenerative disorders.

For a general reference to PKs malfunctioning or deregulation see, for instance, Current Opinion in Chemical Biology, 1999, 3, 459-465; Nature Rev. Drug Discov. 2002; and Carcinogenesis, 2008, 29,1087-1091.

Cyclin-dependent kinase 11 (CDK11) is a serine/threonine kinase encoded by two nearly identical genes, CDC2L1 and CDC2L2. Two predominant CDK11 protein isoforms are produced from mammalian CDK11 gene(s): CDK11p110, that is detected in mammalian tissues and cell lines throughout all the cell cycle, and CDK11p58, that is produced via usage of an internal ribosomal entry site within CDK11 mRNA transcripts at the G2/M cell cycle transition (Trembley et al. 2004). CDK11 acts in complex with cyclins L1 and L2 (CYCL1 and CYCL2; Loyer et al. 2008). CDK11 has been reported to be involved in the regulation of various cellular processes, including cell cycle, transcription, splicing, and chromosome segregation. CDK11 has been observed to play a role in survival and proliferation of different types of human cancers, including breast, colon and cervical cancer, osteosarcoma, melanoma, multiple myeloma and Acute Myelogneous Leukemia Furthermore, inhibition of CDK11 by gene knockout or knockdown has been observed to exart an antitumoral effect in different cancer models, thereby suggesting that targeting CDK11 could result in a therapeutic effect (Zhou et al. 2016, Loyer and Trembley 2020). Indazole derivatives are known in the art as protein kinase inhibitors. Among them, for instance, WO2008/154241, in name of Abbvie Inc., reports 5-heteroaryl substituted indazole useful in the therapy of diseases associated with dysregulated protein kinase activity; WO2003/028720 discloses amino indazole derivatives also active as kinase inhibitors; however none of aforementioned applications report activity data on the Cyclin-dependent kinase 11 (CDK11).

Taking the above into consideration, there is a strong need for the development of CDK11 inhibitors for the treatment of cancer and other diseases. The present inventors have now identified novel 3-amino indazole derivatives endowed with inhibitory activity toward CDK11 protein kinase.

### DETAILED DESCRIPTION OF THE INVENTION

The present inventors have discovered that compounds of general formula (I), as defined below, are kinase inhibitors and in particular are inhibitors of CDK11 families. Due to the key role of protein kinases, in particular of CDK11, in the regulation of cellular proliferation, such compounds are thus useful to treat diseases caused by altered CDK11 activity, in particular in the treatment of cancer as well as in the treatment of a variety of cell proliferative disorders and immune-related disorder.

Accordingly, a first object of the present invention is to provide a substituted 3-amino indazole derivative of formula (I):

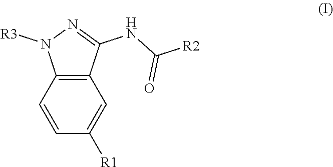

wherein:
R1 is:
optionally substituted straight or branched (C1-C6) alkyl;
optionally substituted (C3-C7) cycloalkyl;
optionally substituted 5 to 7-membered heterocyclyl;
optionally substituted aryl;
5 or 6-membered heteroaryl selected from the group consisting of pyridyl, pyrimidinyl, pyrrolyl and pyrazolyl; or a group of formula (II):

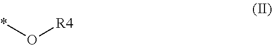

wherein R4 is:
optionally substituted straight or branched (C2-C6) alkyl;
optionally substituted (C3-C7) cycloalkyl; or
optionally substituted 5 to 7-membered heterocyclyl;
R2 is:
straight or branched (C2-C6) alkyl;
optionally substituted (C3-C7) cycloalkyl;
optionally substituted heterocyclyl;
5 to 6-membered heterocyclylmethyl group [—CH2-Het] wherein the heterocyclyl is selected from the group consisting of pirrolydinyl, methylpiperazinyl and piperidinyl; or
a group of formula (III):

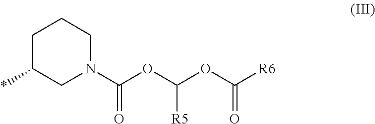

wherein:
R5 is:
hydrogen;
an optionally substituted straight or branched (C1-C6) alkyl;
optionally substituted (C3-C7) cycloalkyl;
optionally substituted aryl;
optionally substituted heterocycly; or
optionally substituted heteroaryl;
R6 is:
an optionally substituted straight or branched (C1-C6) alkyl;
optionally substituted (C3-C7) cycloalkyl;
optionally substituted aryl;
optionally substituted heterocyclyl; or
optionally substituted heteroaryl;
R3 is hydrogen or a group of formula (IV):

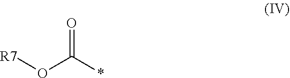

wherein:
R7 is:
an optionally substituted straight or branched (C1-C6) alkyl;
optionally substituted (C3-C7) cycloalkyl;
optionally substituted aryl;
optionally substituted heterocyclyl; or
optionally substituted heteroaryl;
or pharmaceutically acceptable salt thereof;
provided that R1 is not:

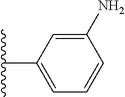 [SMILES: *c1cccc(N)c1]

Preferred compounds of formula (I) are the compound wherein:
R1 is:
mono, di or three substituted phenyl;
optionally substituted 5 or 6-membered heteroaryl selected from the group consisting of pyridyl, pyrrolyl and pyrazolyl; wherein the substituents are each independently selected from the group consisting of halogens, straight or branched (C1-C6) alkyl, alkoxy, hydroxyalkyl, polyfluorinated alkyl, polyfluorinated alkoxy, cyano, amino, alkylsulfonyl, aminosulfonyl, phenyl, hydroxy, aminocarbonyl, alkylaminocarbonyl, hydroxyalkylaminocarbonyl, formyl, formylamino, nitro, alkylcarbonyl, alkyloxycarbonyl and oxadiazolyl; or a group of formula (II):

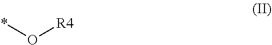

wherein R4 is:
straight or branched (C2-C6) alkyl, C3-C7) cycloalkyl, optionally substituted by halogen or alkoxy;
R2 is:
(C3-C7) cycloalkyl optionally substituted with hydroxy, aminoalkyl, alkylamino, dialkylamino, alkylaminoalkyl, heterocyclyl, C3-C7cycloalkylamino, haloalkylamino;
optionally substituted 5 to 7-membered heterocyclyl with from 1 to 2 heteroatoms selected among N or 0;
1-azabicyclo[2.2.2]octanyl;
2-azabicyclo[2.2.2]octanyl; or
a group of formula (III):

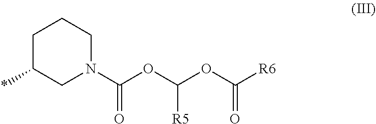

wherein R5 is:
hydrogen;
optionally substituted straight or branched (C1-C6) alkyl; or
optionally substituted (C3-C7) cycloalkyl;
R6 is:
optionally substituted straight or branched (C1-C6) alkyl;
optionally substituted (C3-C7) cycloalkyl;
optionally substituted phenyl;
optionally substituted heterocyclyl; or
optionally substituted 5 or 6-membered heteroaryl with from 1 to 2 nitrogen atoms;
R3 is:
hydrogen or
a group of formula (IV) wherein R7 is:
   straight or branched (C1-C6) alkyl, optionally substituted with (C3-C7) cycloalkyl, alkylthio, alkoxy or alkoxy (C1-C6) alkoxy; or
   (C3-C7) cycloalkyl;
provided that R1 is not

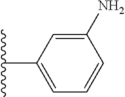 [SMILES: *c1cccc(N)c1]

or a pharmaceutically acceptable salt thereof.

More preferred compounds of formula (I) are the compounds wherein:
R1 is:
a disubstituted phenyl;
optionally substituted 5 or 6-membered heteroaryl selected from the group consisting of pyridyl and pyrazolyl; wherein the substituents are each independently selected from the group consisting of halogens, straight or branched (C1-C6) alkyl alkoxy, hydroxyalkyl, polyfluorinated alkyl, polyfluorinated alkoxy, cyano, amino, alkylsulfonyl, aminosulfonyl, phenyl, hydroxy, aminocarbonyl, alkylaminocarbonyl, hydroxyalkylaminocarbonyl, formyl, formylamino, nitro, alkylcarbonyl, alkyloxycarbonyl and oxadiazolyl; or
a group of formula (II) as described above;
R2 is:
(C3-C7) cycloalkyl selected from the group consisting of cyclopropyl, cyclobutyl and cyclohexyl, optionally substituted with hydroxy, aminoalkyl, alkylamino, dialkylamino, haloalkylamino, alkylaminoalkyl, morpholinyl, (C3-C7)cycloalkylamino;
heterocyclyl selected from the group consisting of pyrrolidinyl, piperidinyl, morfolinyl, tetrahydropyranyl and azepanyl, optionally substituted with straight or branched (C1-C6) alkyl, (C3-C6) cycloalkyl, spiro (C4-C6) cycloalkyl, aminoalkyl, alkoxyalkyl, hydroxyalkyl, phenyl, polyfluorinated alkyl, C3-C7 cycloalkylamino and hydroxy; or
a group of formula (III) as defined above;
R3 and R4 are as defined above;
or a pharmaceutically acceptable salt thereof.

Preferred specific compounds (cpd) of formula (I) or a salt thereof are the compounds listed below: N-[5-(4-Amino-2-chlorophenyl)-1H-indazol-3-yl]piperidine-3-carboxamide dihydrochloride (cpd 1); N-[5-(2,4-Dichlorophenyl)-1H-indazol-3-yl]-1-methylpiperidine-3-carboxamide hydrochloride (cpd 2); N-[5-(2-Chloro-4-hydroxyphenyl)-1H-indazol-3-yl]piperidine-3-carboxamide hydrochloride (cpd 3); N-[5-(2,5-Dichlorophenyl)-1H-indazol-3-yl]piperidine-3-carboxamide hydrochloride (cpd 4); (3R)-N-[5-(4-Amino-2-chlorophenyl)-1H-indazol-3-yl]piperidine-3-carboxamide dihydrochloride (cpd 5); (3R)-N-[5-(2-chloro-4-hydroxyphenyl)-1H-indazol-3-yl]piperidine-3-carboxamide hydrochloride (cpd 6); (3S)-N-[5-(4-Amino-2-chlorophenyl)-1H-indazol-3-yl]piperidine-3-carboxamide dihydrochloride (cpd 7); (3R)-N-[5-(2-Chloro-4-methylphenyl)-1H-indazol-3-yl]piperidine-3-carboxamide hydrochloride (cpd 8); N-[5-(5-Carbamoyl-2-chlorophenyl)-1H-indazol-3-yl]piperidine-3-carboxamide hydrochloride (cpd 9); (3S)-N-[5-(2-chloro-4-hydroxyphenyl)-1H-indazol-3-yl]piperidine-3-carboxamide hydrochloride (cpd 10); N-[5-(3-Amino-2-fluorophenyl)-1H-indazol-3-yl]piperidine-3-carboxamide hydrochloride (cpd 11); (3S)-N-[5-(2-Chloro-4-methylphenyl)-1H-indazol-3-yl]piperidine-3-carboxamide hydrochloride (cpd 12); (3R)-N-[5-(2,5-Dichlorophenyl)-1H-indazol-3-yl]piperidine-3-carboxamide hydrochloride (cpd 13); (3R)-N-[5-(2,4-Dichlorophenyl)-1H-indazol-3-yl]piperidine-3-carboxamide hydrochloride (cpd 14); N-{5-[5-Chloro-2-(trifluoromethyl)phenyl]-1H-indazol-3-yl}piperidine-3-carboxamide trifluoroacetate (cpd 15); N-[5-(5-Acetyl-2-fluorophenyl)-1H-indazol-3-yl]piperidine-3-carboxamide hydrochloride (cpd 16); N-[5-(2-Chloro-5-methoxyphenyl)-1H-indazol-3-yl]piperidine-3-carboxamide hydrochloride (cpd 17); (3R)-N-[5-(2-Chloro-5-methoxyphenyl)-1H-indazol-3-yl]pipeidine-3-carboxamide hydrochloride (cpd 18); (3R)-N-[5-(5-Acetyl-2-fluorophenyl)-1H-indazol-3-yl]piperidine-3-carboxamide hydrochloride (cpd 19); (3R)-N-[5-(2-Fluoro-5-methoxyphenyl)-1H-indazol-3-yl]piperidine-3-carboxamide hydrochloride (cpd 20); (3R)-N-[5-(5-Chloro-2-fluorophenyl)-1H-indazol-3-yl]piperidine-3-carboxamide hydrochloride (cpd 21); (3R)-N-[5-(5-Ethoxy-2-fluorophenyl)-1H-indazol-3-yl]piperidine-3-carboxamide hydrochloride (cpd 22); (3R)-N-{5-[2-Fluoro-5-(trifluoromethoxy)phenyl]-1H-indazol-3-yl}piperidine-3-carboxamide hydrochloride (cpd 23); (3R)-N-[5-(5-Cyano-2-methylphenyl)-1H-indazol-3-yl]piperidine-3-carboxamide hydrochloride (cpd 24); (3R)-N-[5-(5-Cyano-2-fluorophenyl)-1H-indazol-3-yl]piperidine-3-carboxamide hydrochloride (cpd 25); (3R)-N-[5-(2-Chloro-5-nitrophenyl)-1H-indazol-3-yl]piperidine-3-carboxamide hydrochloride (cpd 26); N-[5-(2,6-Dichlorophenyl)-1H-indazol-3-yl]-6-oxopiperidine-3-carboxamide (cpd 27); N-[5-(5-Acetyl-2-fluorophenyl)-1H-indazol-3-yl]-6-oxopiperidine-3-carboxamide (cpd 28); Methyl 4-chloro-3-(3-{[(3R)-piperidin-3-ylcarbonyl]amino}-1H-indazol-5-yl)benzoate hydrochloride (cpd 29); (3R)-N-[5-(2,4-Difluorophenyl)-1H-indazol-3-yl]piperidine-3-carboxamide hydrochloride (cpd 30); (3R)-N-{5-[4-Methoxy-2-(trifluoromethyl)phenyl]-1H-indazol-3-yl}piperidine-3-carboxamide hydrochloride (cpd 31); (3R)-N-[5-(2-Fluoro-5-methylphenyl)-1H-indazol-3-yl]piperidine-3-carboxamide hydrochloride (cpd 32); (3R)-N-[5-(5-Acetyl-2-chlorophenyl)-1H-indazol-3-yl]piperidine-3-carboxamide hydrochloride (cpd 33); (3R)-N-[5-(5-Amino-2-chlorophenyl)-1H-indazol-3-yl]piperidine-3-carboxamide dihydrochloride (cpd 34); (3R)-N-[5-(2,5-Difluorophenyl)-1H-indazol-3-yl]piperidine-3-carboxamide hydrochloride (cpd 35); (3R)-N-{5-[4-Methyl-2-(trifluoromethyl)phenyl]-1H-indazol-3-yl}piperidine-3-carboxamide hydrochloride (cpd 36); Methyl 4-chloro-3-(3-{[(3R)-pyrrolidin-3-ylcarbonyl]amino}-1H-indazol-5-yl)benzoate hydrochloride (cpd 37); (3R)-N-[5-(5-Acetyl-2-chlorophenyl)-1H-indazol-3-yl]pyrrolidine-3-carboxamide hydrochloride (cpd 38); (3R)-N-{5-[2-Chloro-5-(methylcarbamoyl)phenyl]-1H-indazol-3-yl}pyrrolidine-3-carboxamide hydrochloride (cpd 39); Methyl 4-cyano-3-(3-{[(3R)-piperidin-3-ylcarbonyl]amino}-1H-indazol-5-yl)benzoate hydrochloride (cpd 40); (3R)-N-[5-(2-Chloro-5-hydroxyphenyl)-1H-indazol-3-yl]piperidine-3-carboxamide hydrochloride (cpd 41); Methyl 2,4-dichloro-5-(3-{[(3R)-piperidin-3-ylcarbonyl]amino}-1H-indazol-5-yl)benzoate hydrochloride (cpd 42); (3R)-N-[5-(2-Chloro-5-propanoylphenyl)-1H-indazol-3-yl]piperidine-3-carboxamide hydrochloride (cpd 43); (3R)-N-{5-[2-Chloro-4-(trifluoromethyl)phenyl]-1H-indazol-3-yl}piperidine-3-carboxamide hydrochloride (cpd 44); (3R)-N-{5-[4-Chloro-2-(trifluoromethyl)phenyl]-1H-indazol-3-yl}piperidine-3-carboxamide hydrochloride (cpd 45); (3R)-N-{5-[5-Fluoro-2-(trifluoromethyl)phenyl]-1H-indazol-3-yl}piperidine-3-carboxamide hydrochloride (cpd 46); (3R)-N-[5-(2-Chlorophenyl)-1H-indazol-3-yl]piperidine-3-carboxamide hydrochloride (cpd 47); (3R)-N-{5-[2-Chloro-5-(propan-2-yl)phenyl]-1H-indazol-3-yl}piperidine-3-carboxamide hydrochloride (cpd 48); (3R)-N-(5-Phenyl-1H-indazol-3-yl)piperidine-3-carboxamide hydrochloride (cpd 49); Methyl 2,4-difluoro-5-(3-{[(3R)-piperidin-3-ylcarbonyl]amino}-1H-indazol-5-yl)benzoate hydrochloride (cpd 50); (3R)-N-[5-(5-tert-Butyl-2-chlorophenyl)-1H-indazol-3-yl]piperidine-3-carboxamide hydrochloride (cpd 51); (3R)-N-[5-(2-Chloro-5-fluorophenyl)-1H-indazol-3-yl]piperidine-3-carboxamide hydrochloride (cpd 52); (3R)-N-[5-(2,5-Dicyanophenyl)-1H-indazol-3-yl]piperidine-3-carboxamide hydrochloride (cpd 53); Methyl 2-amino-4-chloro-5-(3-{[(3R)-pyrrolidin-3-ylcarbonyl]amino}-1H-indazol-5-yl)benzoate dihydrochloride (cpd 54); (3R)-N-{5-[2-Cyano-5-(methylcarbamoyl)phenyl]-1H-indazol-3-yl}piperidine-3-carboxamide hydrochloride (cpd 55); Propan-2-yl 4-chloro-3-(3-{[(3R)-piperidin-3-ylcarbonyl]amino}-1H-indazol-5-yl)benzoate hydrochloride (cpd 56); Methyl 2,4-dichloro-3-(3-{[(3R)-piperidin-3-ylcarbonyl]amino}-1H-indazol-5-yl)benzoate hydrochloride (cpd 57); (3R)-N-{5-[2-Chloro-5-(1,2,4-oxadiazol-3-yl)phenyl]-1H-indazol-3-yl}piperidine-3-carboxamide dihydrochloride (cpd 58); (3R)-N-{5-[2-Chloro-5-(1,3,4-oxadiazol-2-yl)phenyl]-1H-indazol-3-yl}piperidine-3-carboxamide trifluoroacetate (cpd 59); (3R)-N-{5-[2-Chloro-5-(5-methyl-1,2,4-oxadiazol-3-yl)phenyl]-1H-indazol-3-yl}piperidine-3-carboxamide dihydrochloride (cpd 60); N-[5-(2-Chloro-5-cyanophenyl)-1H-indazol-3-yl]piperidine-4-carboxamide hydrochloride (cpd 61); N-[5-(2-Chloro-4-hydroxyphenyl)-1H-indazol-3-yl]-3-(dimethylamino)cyclobutanecarboxamide hydrochloride (cpd 62); N-[5-(2-Chloro-4-hydroxyphenyl)-1H-indazol-3-yl]-3-(methylamino)cyclobutanecarboxamide hydrochloride (cpd 63); N-[5-(2-Chloro-6-fluorophenyl)-1H-indazol-3-yl]-1-methylpiperidine-4-carboxamide hydrochloride (cpd 64); 1-Butyl-N-[5-(2-chloro-6-fluorophenyl)-1H-indazol-3-yl]piperidine-4-carboxamide hydrochloride (cpd 65); 1-(3-Aminopropyl)-N-[5-(2-chloro-6-fluorophenyl)-1H-indazol-3-yl]piperidine-4-carboxamide dihydrochloride (cpd 66); N-[5-(2-Chloro-6-fluorophenyl)-1H-indazol-3-yl]-1-(1-methoxypropan-2-yl)piperidine-4-carboxamide hydrochloride (cpd 67); N-[5-(2,6-Dichlorophenyl)-1H-indazol-3-yl]-1-methylpiperidine-4-carboxamide (cpd 68); 1-Butyl-N-[5-(2,6-dichlorophenyl)-1H-indazol-3-yl]piperidine-4-carboxamide (cpd 69); N-[5-(2,6-Dichlorophenyl)-1H-indazol-3-yl]-1-(2-methylpropyl)piperidine-4-carboxamide (cpd 70); N-[5-(2,6-Dichlorophenyl)-1H-indazol-3-yl]-1-(3-hydroxy-2,2-dimethylpropyl)piperidine-4-carboxamide (cpd 71); N-[5-(6-Chloro-2-fluoro-3-methylphenyl)-1H-indazol-3-yl]-1-methylpiperidine-4-carboxamide hydrochloride (cpd 72); N-[5-(2-Chloro-6-fluoro-3-methylphenyl)-1H-indazol-3-yl]-1-methylpiperidine-4-carboxamide hydrochloride (cpd 73); N-[5-(5-Chloro-2-fluoro-3-methylphenyl)-1H-indazol-3-yl]-1-methylpiperidine-4-carboxamide hydrochloride (cpd 74); N-{5-[2-Methoxy-5-(propan-2-yl)phenyl]-1H-indazol-3-yl}-1-methylpiperidine-4-carboxamide hydrochloride (cpd 75); 1-Methyl-N-{5-[3-(trifluoromethyl)phenyl]-1H-indazol-3-yl}piperidine-4-carboxamide hydrochloride (cpd 76); N-{5-[2-Chloro-5-(hydroxymethyl)phenyl]-1H-indazol-3-yl}-1-methylpiperidine-4-carboxamide p-toluensulfonate (cpd 77); N-[5-(2-Fluorophenyl)-1H-indazol-3-yl]-1-methylpiperidine-4-carboxamide hydrochloride (cpd 78); N-[5-(2-Cyanophenyl)-1H-indazol-3-yl]-1-methylpiperidine-4-carboxamide p-toluensulfonate (cpd 79); N-[5-(2,5-Dichlorophenyl)-1H-indazol-3-yl]-1-methylpiperidine-4-carboxamide hydrochloride (cpd 80); N-[5-(3-Carbamoylphenyl)-1H-indazol-3-yl]-1-methylpiperidine-4-carboxamide hydrochloride (cpd 81); N-{5-[4-(Hydroxymethyl)phenyl]-1H-indazol-3-yl}-1-methylpiperidine-4-carboxamide p-toluensulfonate (cpd 82); N-{5-[3-(Hydroxymethyl)phenyl]-1H-indazol-3-yl}-1-methylpiperidine-4-carboxamide p-toluensulfonate (cpd 83); 1-Methyl-N-[5-(pyridin-3-yl)-1H-indazol-3-yl]piperidine-4-carboxamide dihydrochloride (cpd 84); N-[5-(2-Chlorophenyl)-1H-indazol-3-yl]-1-methylpiperidine-4-carboxamide hydrochloride (cpd 85); N-[5-(2-Chloro-5-methylphenyl)-1H-indazol-3-yl]-1-methylpiperidine-4-carboxamide hydrochloride (cpd 86); N-[5-(4-Fluorophenyl)-1H-indazol-3-yl]-1-methylpiperidine-4-carboxamide hydrochloride (cpd 87); N-[5-(2-Ethylphenyl)-1H-indazol-3-yl]-1-methylpiperidine-4-carboxamide hydrochloride (cpd 88); N-[5-(3-Cyanophenyl)-1H-indazol-3-yl]-1-methylpiperidine-4-carboxamide hydrochloride (cpd 89); N-[5-(4-Aminophenyl)-1H-indazol-3-yl]-1-methylpiperidine-4-carboxamide dihydrochloride (cpd 90); N-[5-(2-Aminophenyl)-1H-indazol-3-yl]-1-methylpiperidine-4-carboxamide dihydrochloride (cpd 91); 1-Methyl-N-[5-(4-sulfamoylphenyl)-1H-indazol-3-yl]piperidine-4-carboxamide hydrochloride (cpd 92); 1-Methyl-N-(5-phenyl-1H-indazol-3-yl)piperidine-4-carboxamide hydrochloride (cpd 93); N-{5-[2-Chloro-5-(trifluoromethyl)phenyl]-1H-indazol-3-yl}-1-methylpiperidine-4-carboxamide hydrochloride (cpd 94); N-[5-(2-Fluoropyridin-3-yl)-1H-indazol-3-yl]-1-methylpiperidine-4-carboxamide dihydrochloride (cpd 95); N-[5-(2-Methoxyphenyl)-1H-indazol-3-yl]-1-methylpiperidine-4-carboxamide hydrochloride (cpd 96); N-[5-(2,3-Dichlorophenyl)-1H-indazol-3-yl]-1-methylpiperidine-4-carboxamide hydrochloride (cpd 97); N-[5-(2-Chloro-6-methoxyphenyl)-1H-indazol-3-yl]-1-methylpiperidine-4-carboxamide hydrochloride (cpd 98); N-[5-(2,3-Difluorophenyl)-1H-indazol-3-yl]-1-methylpiperidine-4-carboxamide hydrochloride (cpd 99); 1-Methyl-N-{5-[3-(methylsulfonyl)phenyl]-1H-indazol-3-yl}piperidine-4-carboxamide hydrochloride (cpd 100); N-[5-(3-Fluorophenyl)-1H-indazol-3-yl]-1-methylpiperidine-4-carboxamide hydrochloride (cpd 101); N-[5-(2-Hydroxyphenyl)-1H-indazol-3-yl]-1-methylpiperidine-4-carboxamide hydrochloride (cpd 102); N-[5-(Biphenyl-2-yl)-1H-indazol-3-yl]-1-methylpiperidine-4-carboxamide hydrochloride (cpd 103); 1-Methyl-N-{5-[2-(trifluoromethyl)phenyl]-1H-indazol-3-yl}piperidine-4-carboxamide hydrochloride (cpd 104); N-[5-(2,6-Difluorophenyl)-1H-indazol-3-yl]-1-methylpiperidine-4-carboxamide hydrochloride (cpd 105); N-[5-(2,4-Dichlorophenyl)-1H-indazol-3-yl]-1-methylpiperidine-4-carboxamide hydrochloride (cpd 106); N-[5-(4-Amino-2-chlorophenyl)-1H-indazol-3-yl]-1-methylpiperidine-4-carboxamide dihydrochloride (cpd 107); 1-Methyl-N-[5-(2,4,6-trichlorophenyl)-1H-indazol-3-yl]piperidine-4-carboxamide hydrochloride (cpd 108); N-[5-(4-Carbamoylphenyl)-1H-indazol-3-yl]-1-methylpiperidine-4-carboxamide hydrochloride (cpd 109); N-[5-(4-Cyanophenyl)-1H-indazol-3-yl]-1-methylpiperidine-4-carboxamide hydrochloride (cpd 110); N-[5-(2-Chloro-4-hydroxyphenyl)-1H-indazol-3-yl]-1-methylpiperidine-4-carboxamide hydrochloride (cpd 111); N-[5-(4-Amino-3-fluorophenyl)-1H-indazol-3-yl]-1-methylpiperidine-4-carboxamide hydrochloride (cpd 112); N-[5-(4-Cyano-2-methylphenyl)-1H-indazol-3-yl]-1-methylpiperidine-4-carboxamide hydrochloride (cpd 113); N-[5-(2,6-Dimethylphenyl)-1H-indazol-3-yl]-1-methylpiperidine-4-carboxamide trifluoroacetate (cpd 114); N-[5-(2-Fluoro-5-nitrophenyl)-1H-indazol-3-yl]-1-methylpiperidine-4-carboxamide hydrochloride (cpd 115); N-[5-(2-Chloro-5-cyanophenyl)-1H-indazol-3-yl]-1-methylpiperidine-4-carboxamide trifluoroacetate (cpd 116); N-[5-(5-Carbamoyl-2-chlorophenyl)-1H-indazol-3-yl]-1-methylpiperidine-4-carboxamide trifluoroacetate (cpd 117); N-[5-(2-Fluoro-4-formylphenyl)-1H-indazol-3-yl]-1-methylpiperidine-4-carboxamide hydrochloride (cpd 118); N-[5-(2-Fluoro-4-methylphenyl)-1H-indazol-3-yl]-1-methylpiperidine-4-carboxamide hydrochloride (cpd 119); N-[5-(2,6-Difluoro-4-methoxyphenyl)-1H-indazol-3-yl]-1-methylpiperidine-4-carboxamide hydrochloride (cpd 120); 1-Methyl-N-[5-(1H-pyrazol-4-yl)-1H-indazol-3-yl]piperidine-4-carboxamide hydrochloride (cpd 121); 1-Methyl-N-[5-(1-methyl-1H-pyrazol-4-yl)-1H-indazol-3-yl]piperidine-4-carboxamide hydrochloride (cpd 122); 1-Methyl-N-[5-(2,4,6-trifluorophenyl)-1H-indazol-3-yl]piperidine-4-carboxamide hydrochloride (cpd 123); N-[5-(2,4-Difluorophenyl)-1H-indazol-3-yl]-1-methylpiperidine-4-carboxamide hydrochloride (cpd 124); 1-Methyl-N-[5-(1H-pyrazol-3-yl)-1H-indazol-3-yl]piperidine-4-carboxamide hydrochloride (cpd 125); 1-Methyl-N-[5-(2,4,5-trifluorophenyl)-1H-indazol-3-yl]piperidine-4-carboxamide hydrochloride (cpd 126); 1-Methyl-N-{5-[2,4,6-trifluoro-3-(propan-2-yloxy)phenyl]-1H-indazol-3-yl}piperidine-4-carboxamide hydrochloride (cpd 127); 1-Methyl-N-[5-(2,3,4-trifluorophenyl)-1H-indazol-3-yl]piperidine-4-carboxamide hydrochloride (cpd 128); N-[5-(4-fluoro-2-methylphenyl)-1H-indazol-3-yl]-1-methylpiperidine-4-carboxamide trifluoroacetate (cpd 129); N-[5-(4-Amino-2-methylphenyl)-1H-indazol-3-yl]-1-methylpiperidine-4-carboxamide dihydrochloride (cpd 130); 1-Methyl-N-[5-(2,4,6-trifluoro-3-methoxyphenyl)-1H-indazol-3-yl]piperidine-4-carboxamide hydrochloride (cpd 131); N-[5-(2,3-Difluoro-4-methylphenyl)-1H-indazol-3-yl]-1-methylpiperidine-4-carboxamide hydrochloride (cpd 132); N-[5-(2-Chloro-4-fluoro-3-methylphenyl)-1H-indazol-3-yl]-1-methylpiperidine-4-carboxamide hydrochloride (cpd 133); N-[5-(3-Cyano-2,6-difluorophenyl)-1H-indazol-3-yl]-1-methylpiperidine-4-carboxamide (cpd 134); N-[5-(3-Cyano-4-hydroxyphenyl)-1H-indazol-3-yl]-1-methylpiperidine-4-carboxamide hydrochloride (cpd 135); N-[5-(2-Cyano-5-fluorophenyl)-1H-indazol-3-yl]-1-methylpiperidine-4-carboxamide hydrochloride (cpd 136); N-[5-(2-Chloro-5-nitrophenyl)-1H-indazol-3-yl]-1-methylpiperidine-4-carboxamide trifluoroacetate (cpd 137); N-[5-(2,6-Difluoro-4-hydroxyphenyl)-1H-indazol-3-yl]-1-methylpiperidine-4-carboxamide trifluoroacetate (cpd 138); 1-Methyl-N-[5-(2,4,6-trifluoro-3-hydroxyphenyl)-1H-indazol-3-yl]piperidine-4-carboxamide (cpd 139); 1-Butyl-N-[5-(2-chloro-5-cyanophenyl)-1H-indazol-3-yl]piperidine-4-carboxamide hydrochloride (cpd 140); N-[5-(2-Chloro-5-cyanophenyl)-1H-indazol-3-yl]-1-propylpiperidine-4-carboxamide hydrochloride (cpd 141); N-[5-(2-Chloro-5-cyanophenyl)-1H-indazol-3-yl]-1-pentylpiperidine-4-carboxamide hydrochloride (cpd 142); N-[5-(2-Chloro-5-cyanophenyl)-1H-indazol-3-yl]-1-(propan-2-yl)piperidine-4-carboxamide hydrochloride (cpd 143); N-[5-(2,6-Dichlorophenyl)-1H-indazol-3-yl]-4-(methylamino)cyclohexanecarboxamide hydrochloride (cpd 144); N-[5-(2,6-Dichlorophenyl)-1H-indazol-3-yl]-3-(methylamino)cyclobutanecarboxamide hydrochloride (cpd 145); N-[5-(2-Chloro-5-cyanophenyl)-1H-indazol-3-yl]-3-(methylamino)cyclobutanecarboxamide hydrochloride (cpd 146); N-[5-(2-Chloro-5-cyanophenyl)-1H-indazol-3-yl]-3-(dimethylamino)cyclobutanecarboxamide hydrochloride (cpd 147); N-[5-(2-Chloro-5-cyanophenyl)-1H-indazol-3-yl]-3-(propan-2-ylamino)cyclobutanecarboxamide hydrochloride (cpd 148); N-[5-(2-Chloro-5-cyanophenyl)-1H-indazol-3-yl]-3-(morpholin-4-yl)cyclobutanecarboxamide hydrochloride (cpd 149); N-[5-(2-Chloro-5-cyanophenyl)-1H-indazol-3-yl]-3-[(2,2,2-trifluoroethyl)amino]cyclobutanecarboxamide hydro-chloride (cpd 150); N-[5-(2-Chloro-5-cyanophenyl)-1H-indazol-3-yl]-3-[(2-fluoroethyl)amino]cyclobutanecarboxamide hydrochloride (cpd 151); N-[5-(2-Chloro-5-cyanophenyl)-1H-indazol-3-yl]-3-(ethylamino)cyclobutanecarboxamide hydrochloride (cpd 152); N-[5-(2-Chloro-5-cyanophenyl)-1H-indazol-3-yl]-3-(cyclobutylamino)cyclobutanecarboxamide hydrochloride (cpd 153); N-[5-(2-Chloro-5-cyanophenyl)-1H-indazol-3-yl]-3-(cyclopentylamino)cyclobutanecarboxamide hydrochloride (cpd 154); N-[5-(2-Chloro-5-cyanophenyl)-1H-indazol-3-yl]-3-(cyclopropylamino)cyclobutanecarboxamide hydrochloride (cpd 155); N-[5-(2,6-Dichlorophenyl)-1H-indazol-3-yl]tetrahydro-2H-pyran-4-carboxamide (cpd 156); N-[5-(2,6-Dichlorophenyl)-1H-indazol-3-yl]piperidine-3-carboxamide hydrochloride (cpd 157); 1-Cyclopropyl-N-[5-(2,6-dichlorophenyl)-1H-indazol-3-yl]piperidine-4-carboxamide hydrochloride (cpd 158); N-[5-(2,6-Dichlorophenyl)-1H-indazol-3-yl]-1-phenylpiperidine-4-carboxamide hydrochloride (cpd 159); N-[5-(2,6-Dichlorophenyl)-1H-indazol-3-yl]-1-azabicyclo[2.2.2]octane-4-carboxamide (cpd 160); N-[5-(2-Chloro-6-fluorophenyl)-1H-indazol-3-yl]-1-methylpiperidine-3-carboxamide hydrochloride (cpd 161); N-[5-(2,6-Dichlorophenyl)-1H-indazol-3-yl]-2-[(2S)-pyrrolidin-2-yl]acetamide hydrochloride (cpd 162); N-[5-(2,6-Dichlorophenyl)-1H-indazol-3-yl]-2-(piperidin-4-yl)acetamide hydrochloride (cpd 163); trans-4-(Aminomethyl)-N-[5-(2,6-dichlorophenyl)-1H-indazol-3-yl]cyclohexanecarboxamide hydrochloride (cpd 164); N-[5-(2-Chloro-6-fluorophenyl)-1H-indazol-3-yl]piperidine-3-carboxamide hydrochloride (cpd 165); N-[5-(2,6-Dichlorophenyl)-1H-indazol-3-yl]-4-(dimethylamino)cyclohexanecarboxamide hydrochloride (cpd 166); (3R)-N-[5-(2,6-Dichlorophenyl)-1H-indazol-3-yl]pyrrolidine-3-carboxamide hydrochloride (cpd 167); (3S)-N-[5-(2,6-Dichlorophenyl)-1H-indazol-3-yl]pyrrolidine-3-carboxamide hydrochloride (cpd 168); (3R)-N-[5-(2,6-Dichlorophenyl)-1H-indazol-3-yl]piperidine-3-carboxamide hydrochloride (cpd 169); (3S)-N-[5-(2,6-Dichlorophenyl)-1H-indazol-3-yl]piperidine-3-carboxamide hydrochloride (cpd 170); N-[5-(2-Chloro-5-cyanophenyl)-1H-indazol-3-yl]piperidine-3-carboxamide hydrochloride (cpd 171); (3R)-N-[5-(2-Chloro-6-fluorophenyl)-1H-indazol-3-yl]piperidine-3-carboxamide hydrochloride (cpd 172); (3S)-N-[5-(2-Chloro-6-fluorophenyl)-1H-indazol-3-yl]piperidine-3-carboxamide hydrochloride (cpd 173); (3R)-N-[5-(2-Chloro-5-cyanophenyl)-1H-indazol-3-yl]piperidine-3-carboxamide hydrochloride (cpd 174); N-[5-(2,6-Dichlorophenyl)-1H-indazol-3-yl]-b-alaninamide hydrochloride (cpd 175); (3R)-N-[5-(2-Chloro-5-cyanophenyl)-1H-indazol-3-yl]pyrrolidine-3-carboxamide hydrochloride (cpd 176); (3R,6S)-N-[5-(2,6-Dichlorophenyl)-1H-indazol-3-yl]-6-methylpiperidine-3-carboxamide trifluoroacetate (cpd 177); (3R,6R)-N-[5-(2,6-Dichlorophenyl)-1H-indazol-3-yl]-6-methylpiperidine-3-carboxamide trifluoroacetate (cpd 178); (3R,6R)-N-[5-(2-Chloro-5-cyanophenyl)-1H-indazol-3-yl]-6-methylpiperidine-3-carboxamide trifluoroacetate (cpd 179); (3R,6S)-N-[5-(2-Chloro-5-cyanophenyl)-1H-indazol-3-yl]-6-(trifluoromethyl)piperidine-3-carboxamide (cpd 180); (3S)-N-[5-(2-Chloro-5-cyanophenyl)-1H-indazol-3-yl]piperidine-3-carboxamide hydrochloride (cpd 181); (2R)-N-[5-(2-Chloro-5-cyanophenyl)-1H-indazol-3-yl]morpholine-2-carboxamide hydrochloride (cpd 182); N-[5-(2-Chloro-5-cyanophenyl)-1H-indazol-3-yl]azepane-3-carboxamide hydrochloride (cpd 183); N-[5-(2-Chloro-5-cyanophenyl)-1H-indazol-3-yl]-6,6-dimethylpiperidine-3-carboxamide hydrochloride (cpd 184); N-[5-(2-Chloro-5-cyanophenyl)-1H-indazol-3-yl]-6-azaspiro[4.5]decane-8-carboxamide hydrochloride (cpd 185); N-[5-(2-Chloro-5-cyanophenyl)-1H-indazol-3-yl]-1-azaspiro[5.5]undecane-3-carboxamide hydrochloride (cpd 186); N-[5-(2-Chloro-5-cyanophenyl)-1H-indazol-3-yl]-2-azabicyclo[2.2.2]octane-4-carboxamide hydrochloride (cpd 187); N-[5-(2-Chloro-5-cyanophenyl)-1H-indazol-3-yl]-1-azabicyclo[2.2.2]octane-3-carboxamide hydrochloride (cpd 188); 1-[(2,2-Dimethylpentanoyl)oxy]ethyl (3R)-3-{[5-(2-chloro-5-cyanophenyl)-1H-indazol-3-yl]carbamoyl}-piperidine-1-carboxylate (cpd 189); 1-[(2-Methylpropanoyl)oxy]ethyl (3R)-3-{[5-(2-chloro-5-cyanophenyl)-1H-indazol-3-yl]carbamoyl}piperidine-1-carboxylate (cpd 190); (Acetyloxy)methyl (3R)-3-{[5-(2-chloro-5-cyanophenyl)-1H-indazol-3-yl]carbamoyl}piperidine-1-carboxylate (cpd 191); [(2-Methylpropanoyl)oxy]methyl (3R)-3-{[5-(2-chloro-5-cyanophenyl)-1H-indazol-3-yl]carbamoyl}piperidine-1-carboxylate (cpd 192); [(2,2-Dimethylpropanoyl)oxy]methyl (3R)-3-{[5-(2-chloro-5-cyanophenyl)-1H-indazol-3-yl]carbamoyl}-piperidine-1-carboxylate (cpd 193); 1-(Acetyloxy)ethyl (3R)-3-{[5-(2-chloro-5-cyanophenyl)-1H-indazol-3-yl]carbamoyl}piperidine-1-carboxylate (cpd 194); 1-[(2,2-Dimethylpropanoyl)oxy]ethyl (3R)-3-{[5-(2-chloro-5-cyanophenyl)-1H-indazol-3-yl]carbamoyl}-piperidine-1-carboxylate (cpd 195); 2-Methyl-1-[(2-methylpropanoyl)oxy]propyl (3R)-3-{[5-(2-chloro-5-cyanophenyl)-1H-indazol-3-yl]carbamoyl}-piperidine-1-carboxylate (cpd 196); 1-[(2,2-Dimethylpropanoyl)oxy]-2-methylpropyl (3R)-3-{[5-(2-chloro-5-cyanophenyl)-1H-indazol-3-yl]carbamoyl}-piperidine-1-carboxylate (cpd 197); 1-{[(1-Methylcyclohexyl)carbonyl]oxy)ethyl (3R)-3-{[5-(2-chloro-5-cyanophenyl)-1H-indazol-3-yl]carbamoyl}-piperidine-1-carboxylate (cpd 198); 1-[(Tetrahydro-2H-pyran-4-ylcarbonyl)oxy]ethyl (3R)-3-{[5-(2-chloro-5-cyanophenyl)-1H-indazol-3-yl]carbamoyl}piperidine-1-carboxylate (cpd 199); 1-[(2,2-Dimethylpropanoyl)oxy]propyl (3R)-3-{[5-(2-chloro-5-cyanophenyl)-1H-indazol-3-yl]carbamoyl}-piperidine-1-carboxylate (cpd 200); N-[5-(2,6-Dichlorophenyl)-1H-indazol-3-yl]-4-hydroxycyclohexanecarboxamide (cpd 201); (+)-N-[5-(2-Chloro-5-cyanophenyl)-1H-indazol-3-yl]-6,6-dimethylpiperidine-3-carboxamide (cpd 202); (−)-N-[5-(2-Chloro-5-cyanophenyl)-1H-indazol-3-yl]-6,6-dimethylpiperidine-3-carboxamide (cpd 203); (8+)-N-[5-(2-Chloro-5-cyanophenyl)-1H-indazol-3-yl]-6-azaspiro[4.5]decane-8-carboxamide hydrochloride (cpd 204); (8-)-N-[5-(2-Chloro-5-cyanophenyl)-1H-indazol-3-yl]-6-azaspiro[4.5]decane-8-carboxamide hydrochloride (cpd 205); (3+)-N-[5-(2-Chloro-5-cyanophenyl)-1H-indazol-3-yl]-1-azaspiro[5.5]undecane-3-carboxamide (cpd 206); (3-)-N-[5-(2-Chloro-5-cyanophenyl)-1H-indazol-3-yl]-1-azaspiro[5.5]undecane-3-carboxamide (cpd 207); Butyl 5-(2-chloro-5-cyanophenyl)-3-{[(3R)-piperidin-3-ylcarbonyl]amino}-1H-indazole-1-carboxylate hydrochloride (cpd 208); Ethyl 5-(2-chloro-5-cyanophenyl)-3-{[(3R)-piperidin-3-ylcarbonyl]amino}-1H-indazole-1-carboxylate hydrochloride (cpd 209); Propan-2-yl 5-(2-chloro-5-cyanophenyl)-3-{[(3R)-piperidin-3-ylcarbonyl]amino}-1H-indazole-1-carboxylate hydrochloride (cpd 210); Hexyl 5-(2-chloro-5-cyanophenyl)-3-{[(3R)-piperidin-3-ylcarbonyl]amino}-1H-indazole-1-carboxylate hydrochloride (cpd 211); 2,2-Dimethylpropyl 5-(2-chloro-5-cyanophenyl)-3-{[(3R)-piperidin-3-ylcarbonyl]amino}-1H-indazole-1-carboxylate hydrochloride (cpd 212); 2-Methylpropyl 5-(2-chloro-5-cyanophenyl)-3-{[(3R)-piperidin-3-ylcarbonyl]amino}-1H-indazole-1-carboxylate hydrochloride (cpd 213); Pentyl 5-(2-chloro-5-cyanophenyl)-3-{[(3R)-piperidin-3-ylcarbonyl]amino}-1H-indazole-1-carboxylate hydrochloride (cpd 214); 2-Methoxyethyl 5-(2-chloro-5-cyanophenyl)-3-{[(3R)-piperidin-3-ylcarbonyl]amino}-1H-indazole-1-carboxylate hydrochloride (cpd 215); Cyclobutyl 5-(2-chloro-5-cyanophenyl)-3-{[(3R)-piperidin-3-ylcarbonyl]amino}-1H-indazole-1-carboxylate hydrochloride (cpd 216); Pentan-3-yl 5-(2-chloro-5-cyanophenyl)-3-{[(3R)-piperidin-3-ylcarbonyl]amino}-1H-indazole-1-carboxylate hydrochloride (cpd 217); Cyclohexylmethyl 5-(2-chloro-5-cyanophenyl)-3-{[(3R)-piperidin-3-ylcarbonyl]amino}-1H-indazole-1-carboxylate hydrochloride (cpd 218); Cyclopropylmethyl 5-(2-chloro-5-cyanophenyl)-3-{[(3R)-piperidin-3-ylcarbonyl]amino}-1H-indazole-1-carboxylate formate (cpd 219); 2-(Ethylsulfanyl)ethyl 5-(2-chloro-5-cyanophenyl)-3-{[(3R)-piperidin-3-ylcarbonyl]amino}-1H-indazole-1-carboxylate formate (cpd 220); 3-(3-Methoxypropoxy)propyl 5-(2-chloro-5-cyanophenyl)-3-{[(3R)-piperidin-3-ylcarbonyl]amino}-1H-indazole-1-carboxylate formate (cpd 221); 4-Methylpentyl 5-(2-chloro-5-cyanophenyl)-3-{[(3R)-piperidin-3-ylcarbonyl]amino}-1H-indazole-1-carboxylate formate (cpd 222); Cyclopentyl 5-(2-chloro-5-cyanophenyl)-3-{[(3R)-piperidin-3-ylcarbonyl]amino}-1H-indazole-1-carboxylate formate (cpd 223); 2,2-Dimethylpropyl 5-(2-chloro-5-cyanophenyl)-3-({[(3+)-6,6-dimethylpiperidin-3-yl]carbonyl}amino)-1H-indazole-1-carboxylate hydrochloride (cpd 224); Hexyl 5-(2-chloro-5-cyanophenyl)-3-({[(3R)-6,6-dimethylpiperidin-3-yl]carbonyl}amino)-1H-indazole-1-carboxylate hydrochloride (cpd 225); Propan-2-yl 5-(2-chloro-5-cyanophenyl)-3-({[(3R)-6,6-dimethylpiperidin-3-yl]carbonyl}amino)-1H-indazole-1-carboxylate hydrochloride (cpd 226); (3R)-N-[5-(2-Fluoroethoxy)-1H-indazol-3-yl]piperidine-3-carboxamide hydrochloride (cpd 227); (3R)-N-[5-(2-Methoxyethoxy)-1H-indazol-3-yl]piperidine-3-carboxamide hydrochloride (cpd 228); (3R)-N-(5-Ethoxy-1H-indazol-3-yl)piperidine-3-carboxamide hydrochloride (cpd 229); (3R)-N-[5-(Propan-2-yloxy)-1H-indazol-3-yl]piperidine-3-carboxamide hydrochloride (cpd 230); and (3R)-N-(5-Butoxy-1H-indazol-3-yl)piperidine-3-carboxamide hydrochloride (cpd 231).

For a reference to any specific compound of formula (I) of the invention, optionally in the form of a pharmaceutically acceptable salt, see the experimental section and claims.

Where compounds of the formula (I) contain one or more chiral centers, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterized and identified by their optical activity (i.e. as + and − isomers, or d and l isomers) or they may be characterized in terms of their absolute stereochemistry using the “R and S” nomenclature developed by Cahn, Ingold and Prelog, see Advanced Organic Chemistry by Jerry March, 4th Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centers, wherein at least 55% (e.g. at least 60%, 65%, 70%, 75%, 80%, 85%, 90% or 95%) of the compound of the formula (I) is present as a single optical isomer (e.g. enantiomer or diastereoisomer).

In cases in which compounds have unsaturated carbon-carbon double bonds, both the cis (Z) and trans (E) isomers are within the scope of this invention.

In cases wherein compounds may exist in tautomeric forms, such as keto-enol tautomers, each tautomeric form is contemplated as being included within this invention whether existing in equilibrium or predominantly in one form.

As such, unless otherwise provided, when in compounds of formula (I) R3 is hydrogen, and only one of the following tautomeric forms of formula (Ia) or (Ib) is indicated, the remaining one has still to be intended as comprised within the scope of the invention:

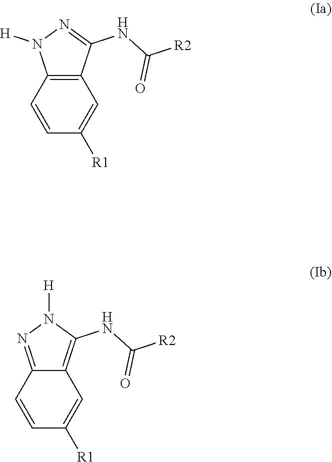

Pharmaceutically acceptable salts of the compounds of formula (I) include the salts with inorganic or organic acids, e.g. nitric, hydrochloric, hydrobromic, sulfuric, perchloric, phosphoric, acetic, trifluoroacetic, formic, propionic, glycolic, lactic, oxalic, fumaric, malonic, malic, maleic, tartaric, citric, benzoic, cinnamic, mandelic, methanesulphonic, isethionic and salicylic acid.

Pharmaceutically acceptable salts of the compounds of formula (I) also include the salts with inorganic or organic bases, e.g. alkali or alkaline-earth metals, especially sodium, potassium, calcium, ammonium or magnesium hydroxides, carbonates or bicarbonates, acyclic or cyclic amines.

Further object of the present invention are compounds of formula (I) as defined above, as well as their isomers, tautomers, hydrates, solvates, complexes, metabolites, prodrugs, carriers and N-oxides.

A metabolite of a compound of formula (I) is any compound into which this same compound of formula (I) is converted in vivo, for instance upon administration to a mammal in need thereof. Typically, without however representing a limiting example, upon administration of a compound of formula (I), this same derivative may be converted into a variety of compounds, for instance including more soluble derivatives like hydroxylated derivatives, which are easily excreted. Hence, depending upon the metabolic pathway thus occurring, any of these hydroxylated derivatives may be regarded as a metabolite of the compounds of formula (I).

Prodrugs are any covalently bound compounds, which release in vivo the active parent drug according to formula (I). N-oxides are compounds of formula (I) wherein nitrogen and oxygen are tethered through a dative bond. Further object of the present invention are compounds of formula (I) wherein one or more hydrogen/s is/are replaced by one or more deuterium atom/s.

With the term “straight or branched (C1-C6) alkyl”, hence comprehensive of (C1-C4) alkyl, we intend any of the groups such as, for instance, methyl, ethyl, n-propyl, isopropyl, n-butyl, isobutyl, tert-butyl, sec-butyl, n-pentyl, n-hexyl, and the like.

The terms “carbocycle”, “carbocyclic ring” and “cycloalkyl” refer to a monovalent non-aromatic, saturated or partially unsaturated ring having 3 to 7 carbon atoms (C3-C7) as monocyclic ring or 7 to 12 carbon atoms (C7-C12) as a bicyclic ring. Bicyclic carbocycles having 7 to 12 atoms can be arranged, for example, as a bicyclo[4,5], [5,5], [5,6] or [6,6] system, and bicyclic carbocycles having 9 or 10 ring atoms can be arranged as a bicyclo[5,6] or [6,6] system, or as bridged systems such as bicyclo[2.2.1]heptane, bicyclo[2.2.2]octane and bicyclo[3.2.2]nonane. Examples of monocyclic carbocycles having 3 to 7atoms include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

The term “aryl” refers to a mono-, bi- or poly-carbocyclic hydrocarbon with from 1 to 4 ring systems, optionally further fused or linked to each other by single bonds, wherein at least one of the carbocyclic rings is “aromatic”, wherein the term “aromatic” refers to completely conjugated π-electron bond system. Non limiting examples of such aryl groups are phenyl, α- or β-naphthyl, α- or β-tetrahydronaphthalenyl, biphenyl, and indanyl groups.

The term “heteroaryl” refers to aromatic heterocyclic rings, typically 5- to 7-membered heterocycles with from 1 to 3 heteroatoms selected among N, O or S; the heteroaryl ring can be optionally further fused or linked to aromatic and non-aromatic carbocyclic and heterocyclic rings. Not limiting examples of such heteroaryl groups are, for instance, pyridyl, pyrazinyl, pyrimidinyl, pyridazinyl, indolyl, imidazolyl, thiazolyl, isothiazolyl, pyrrolyl, furanyl, oxazolyl, isoxazolyl, pyrazolyl, thiophenyl, thiadiazolyl, isoxazolyl, isothiazolyl, oxadiazolyl, indazolyl, cinnolinyl, benzo[1,3]dioxolyl, benzo[1,4]dioxinyl, benzothiazolyl, benzothiophenyl, benzofuranyl, isoindolinyl, benzoimidazolyl, benzoxazolyl, quinolinyl, isoquinolinyl, 1,2,3-triazolyl, 1-phenyl-1,2,3-triazolyl, 2,3-dihydroindolyl, 2,3-dihydrobenzofuranyl, 2,3-dihydrobenzothiophenyl, benzopyranyl, 2,3-dihydrobenzoxazinyl, 2,3-dihydroquinoxalinyl and the like.

With the term “heterocyclyl”, and “heterocyclyle”, we intend a 3- to 7-membered, saturated or partially unsaturated carbocyclic ring where one or more carbon atoms are replaced by heteroatoms such as nitrogen, oxygen and sulfur. Non limiting examples of heterocyclyl groups are, for instance, pyranyl, tetrahydropyranyl, pyrrolidinyl, pyrrolinyl, imidazolinyl, imidazolidinyl, pyrazolidinyl, pyrazolinyl, thiazolinyl, thiazolidinyl, dihydrofuranyl, tetrahydrofuranyl, tetrahydropyridinyl, 1,3-dioxolanyl, piperidinyl, piperazinyl, morpholinyl, dihydroazepinyl, tetrahydroazepinyl, azepanyl, oxadiazole and the like. A heterocycle may be also a bicycle having 7 to 10 ring members (4 to 9 carbon atoms and 1 to 6 heteroatoms selected from N, O, P, and S), for example: a bicyclo[4,5], [5,5], [5,6], or [6,6] system. Not limiting examples of such heterocyclyl groups are 3-azabicyco[3.1.0]hexanyl, 3-azabicyclo[4.1.0]heptanyl, azabicyclo[2.2.2]hexanyl, 1-azabicyclo[2.2.2]octanyl, 2-azabicyclo[2.2.2]octanyl. Spiro moieties are also included within the scope of this definition.

According to the present invention and unless otherwise provided, any of the above R1, R2, R3, R4, R5, R6 and R7, groups may be optionally substituted, in any of their free positions, by one or more groups, for instance 1 to 6 groups, independently selected from: halogen, nitro, oxo groups (═O), cyano, (C1-C6) alkyl, polyfluorinated alkyl, polyfluorinated alkoxy, haloalkyl, haloalkylamino, alkenyl, alkynyl, hydroxyalkyl, aryl, arylalkyl, heterocyclyl, (C3-C7) cycloalkyl, hydroxy, alkoxy, alkoxyalkyl, alkoxy (C1-C6) alkoxy, aryloxy, heteroaryl, heterocyclyloxy, methylenedioxy, alkylcarbonyloxy, arylcarbonyloxy, cycloalkenyloxy, C3-C7 cycloalkylamino, heterocyclylcarbonyloxy, alkylideneaminooxy, carboxy, alkoxycarbonyl, aryloxycarbonyl, cycloalkyloxycarbonyl, heterocyclyloxycarbonyl, amino, ureido, aminoalkyl, alkylamino, dialkylamino, arylamino, diarylamino, heterocyclylamino, formylamino, alkylcarbonylamino, arylcarbonylamino, heterocyclylcarbonylamino, aminocarbonyl, alkylaminocarbonyl, alkylaminoalkyl, dialkylaminocarbonyl, arylaminocarbonyl, heterocyclylaminocarbonyl, alkoxycarbonylamino, hydroxyalkylaminocarbonyl, hydroxyaminocarbonyl alkoxyimino, alkylsulfonylamino, arylsulfonylamino, heterocyclylsulfonylamino, formyl, alkylcarbonyl, arylcarbonyl, cycloalkylcarbonyl, heterocyclylcarbonyl, alkylsulfonyl, arylsulfonyl, aminosulfonyl, alkylaminosulfonyl, dialkylaminosulfonyl, arylaminosulfonyl, heterocyclylaminosulfonyl, arylthio, alkylthio, phosphonate and alkylphosphonate.

In their turn, whenever appropriate, each of the above substituent may be further substituted by one or more of the aforementioned groups.

In this respect, with the term “halogen” we intend a fluorine, chlorine, bromine or iodine atom.

With the term “cyano” we intend a —CN residue.

With the term “nitro” we intend a —NO₂ group.

With the term “alkenyl” or “alkynyl” we intend any of the aforementioned straight or branched (C2-C6) alkyl groups further bearing a double or triple bond. Non limiting examples of alkenyl or alkynyl groups of the invention are, for instance, vinyl, allyl, 1-propenyl, isopropenyl, 1-butenyl, 2-butenyl, 3-butenyl, 2-pentenyl, 1-hexenyl, ethynyl, 2-propynyl, 4-pentynyl, and the like.

With the term “polyfluorinated alkyl or alkoxy” we intend any of the above straight or branched (C1-C6) alkyl or alkoxy groups which are substituted by more than one fluorine atom such as, for instance, trifluoromethyl, trifluoroethyl, 1,1,1,3,3,3-hexafluoropropyl, trifluoromethoxy and the like.

With the terms “alkoxy, aryloxy, heterocyclyloxy” and derivatives thereof, we intend any of the above (C1-C6) alkyl, aryl or heterocyclyl groups linked to the rest of the molecule through an oxygen atom (—O—).

From all of the above, it is clear to the skilled person that any group whose name is a composite name such as, for instance, arylamino, has to be intended as conventionally construed by the parts from which it derives, e.g. by an amino group which is further substituted by aryl, wherein aryl is as above defined.

Likewise, any of the terms such as, for instance, alkylthio, alkylamino, dialkylamino, alkoxycarbonyl, alkoxycarbonylamino, heterocyclylcarbonyl, heterocyclylcarbonylamino, cycloalkyloxycarbonyl and the like, include groups wherein the alkyl, alkoxy, aryl, (C3-C7) cycloalkyl and heterocyclyl moieties are as above defined.

The present invention also provides a process for the preparation of a compound of formula (I) as defined above, by using the reaction routes and synthetic schemes described below, employing the techniques available in the art and starting materials readily available. The preparation of certain embodiments of the present invention is described in the examples that follow, but those of ordinary skill in the art will recognize that the preparations described may be readily adapted to prepare other embodiments of the present invention. For example, the synthesis of non-exemplified compounds according to the invention may be performed by modifications apparent to those skilled in the art, for instance, by appropriately protecting interfering groups, by changing to other suitable reagents known in the art, or by making routine modifications of reaction conditions. Alternatively, other reactions referred to herein or known in the art will be recognized as having adaptability for preparing other compounds of the invention.

The compounds of this invention can be prepared from readily available starting materials using the following general methods and procedures. Unless otherwise indicated, the starting materials are known compounds or may be prepared from known compounds according to well known procedures. It will be appreciated that, where typical or preferred process conditions (i.e., reaction temperatures, times, mole ratios of reactants, solvents, pressures) are described, different process conditions can also be used unless otherwise stated. Optimum reaction conditions may vary with the particular reactants or solvent used, but such conditions can be determined by one skilled in the art by routine optimization procedures.

Compounds of formula (I) can be prepared as reported in Scheme 1 below:

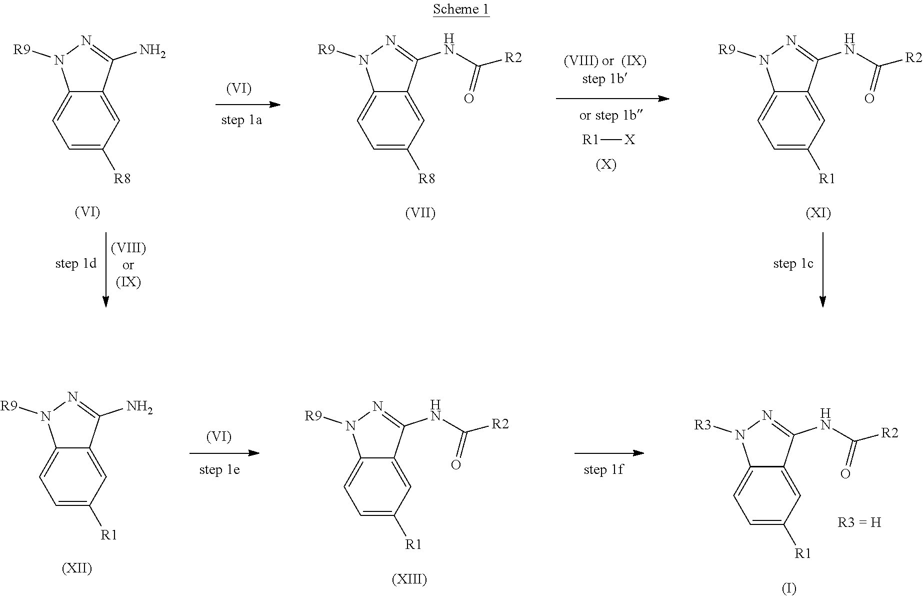

Accordingly, the process of the present invention comprises the following steps:
Step 1a) reacting a compound of formula (V):

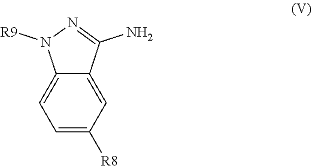

wherein R8 is bromine or 4,4,5,5-tetramethyl-[1,3,2]dioxaborolane and R9 is a suitable protecting group such as 1,1′,1″-methanetriyltribenzene, tert-butyloxycarbonyl, p-metossitritile, 1-(diphenylmethyl)-4-methoxybenzene or tetrahydro-2H-pyranyl (THP) with a compound of formula (VI):

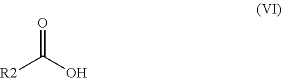

wherein R2 is an optionally substituted straight or branched (C2-C6) alkyl, (C3-C7) cycloalkyl, heterocyclyl, 5 to 6-membered heterocyclylmethyl wherein the heterocyclyl is selected from the group consisting of pirrolydinyl, methylpiperazinyl and piperidinyl;
Step 1b′) mixing the obtained intermediate of formula (VII):

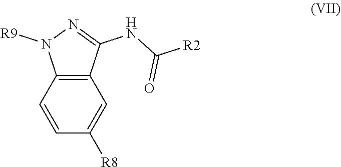

wherein R2 and R9 are defined as above in step 1a and R8 is bromine, with a compound of formula (VIII) or (IX):

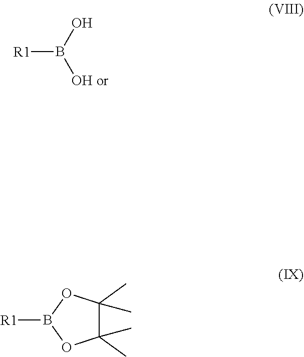

wherein R1 is an optionally substituted straight or branched (C1-C6) alkyl, (C3-C7) cycloalkyl, heterocyclyl, aryl and 5 or 6 membered heteroaryl;
or
Step 1b″) reacting the intermediate of formula (VII) wherein R8 is 4,4,5,5-tetramethyl-[1,3,2]dioxaborolane and R2 and R9 are defined above in step 1a with a compound of formula (X):

R1-X (X)
wherein R1 is defined as above in step 1b′ and X is halogen;
Step 1c) reacting the obtained intermediate (XI):

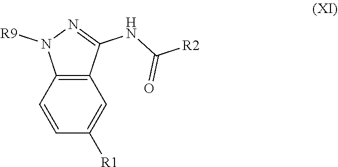

wherein R1, R2 and R9 are defined as above in step 1b′ and 1b″, with the appropriate deprotecting agent to obtain a compound of formula (I):

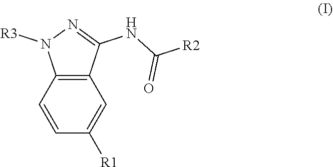

wherein R 3 is hydrogen and R1 and R2 are defined as above;
or
Step 1d) reacting a compound of formula (V):

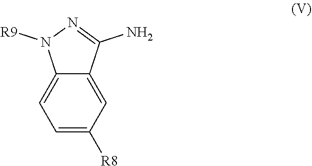

wherein R8 is bromine and R9 is as defined above in step 1a, with a compound of formula (VIII) or (IX):

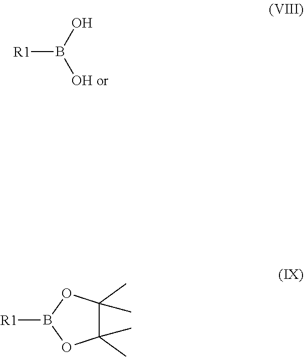

wherein R1 is defined as above in step 1b′;
Step 1e) mixing the resulted intermediate (XII):

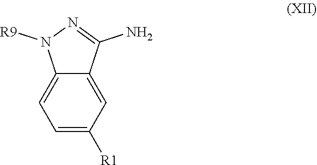

wherein R1 and R9 are as defined above in step 1c, with a compound of formula (VI):

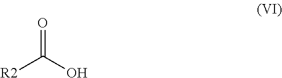

wherein R2 is defined as above in step 1a;
Step 1f) reacting the obtained intermediate (XIII):

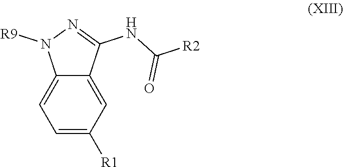

wherein R1, R2 and R9 are defined as above, with the appropriate deprotecting agent thus to obtain a compound of formula (I):

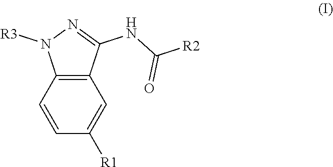

wherein R3 is hydrogen and R1 and R2 are defined as above in step 1c.

Alternatively, if desired, a first compound of formula (VII) or (XII) can be converted into a second compound of formula (VII) or (XII), respectively, by operating according to well-known synthetic conditions.

Examples of possible conversions are those reported below:
conv. A) converting a compound of formula (VIIa):

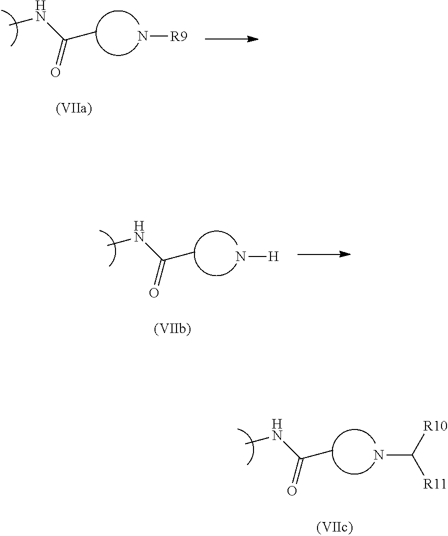

wherein R2 is an optionally substituted heterocyclyl and R9 is defined as above in step 1a, into a compound of formula (VIIb), by first removing the protecting group then, reacting the resulting derivative with a compound of formula R10R11CO (XIV), wherein R10 and R11 are hydrogen, an optionally substituted group selected from straight or branched (C1-C6) alkyl, (C3-C7) cycloalkyl or R10 and R11 taken together may form an optionally substituted (C3-C7) cycloalkyl and heterocyclyl group, under reductive amination conditions in the presence of a suitable reducing agents;
conv. B) converting a compound of formula (VIId):

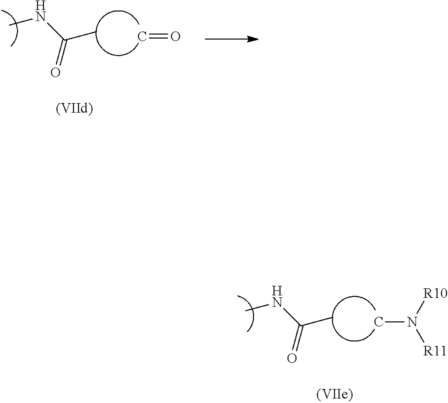

wherein R2 is a (C4-C6) cycloalkyloxo, into a compound of formula (VIIe), by reacting with a compound of formula R10R11NH (XV) wherein R10 and R11 are defined as above in conv. A, under reductive amination conditions in the presence of a suitable reducing agents;
conv. C) converting a compound of formula (XIIIa):

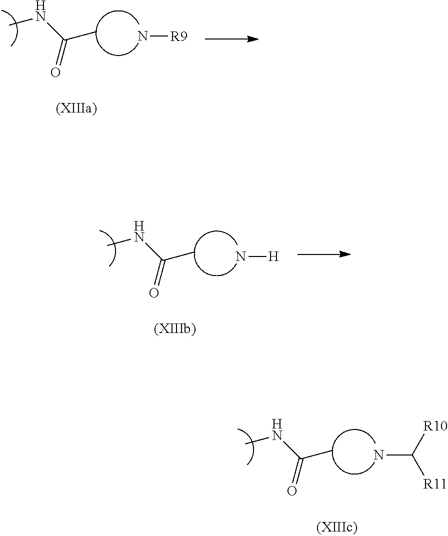

wherein R2 is an optionally substituted heterocycly and R9 is defined as above in step 1a, into a compound of formula (XIIIb), by first removing the protecting group then, reacting the resulting derivative with a compound of formula R10R11CO (XIV), wherein R10 and R11 are defined as above in conv. A, under reductive amination conditions in the presence of a suitable reducing agents;
conv. D) converting a compound of formula (XIIId):

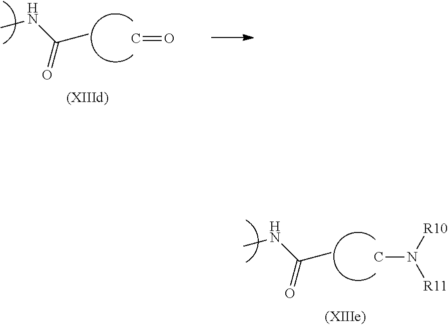

wherein R2 is a (C4-C6) cycloalkyloxo, into a compound of formula (XIIIe), by reacting with a compound of formula R10R11NH (XV) wherein R10 and R11 are defined as above in conv.A, under reductive amination conditions in the presence of a suitable reducing agents;
conv. E) converting a compound of formula (XIIIf):

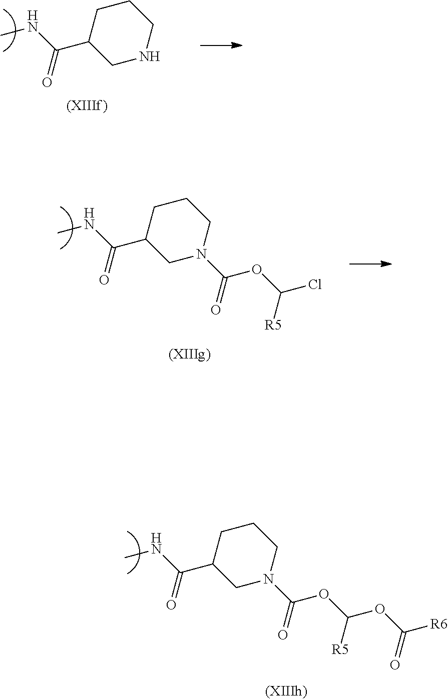

first into a compound of formula (XIIIg), reacting with a compound of formula R5CHClOCOCl (XVI) wherein R5 is hydrogen, an optionally substituted straight or branched (C1-C6) alkyl, (C3-C7) cycloalkyl, aryl, heterocyclyl and heteroaryl, then into a compound of formula (XIIIh) reacting under basic condition with a compound of formula R6COOH (XVII), wherein R6 is an optionally substituted straight or branched (C1-C6) alkyl, (C3-C7) cycloalkyl, aryl, heterocyclyl or heteroaryl.

A first compound of general formula (I) can be conveniently converted into a second compound of formula (I) by operating according to well-known synthetic conditions.

Examples of possible conversions are those reported below:
conv. 1) converting a compound of formula (I):

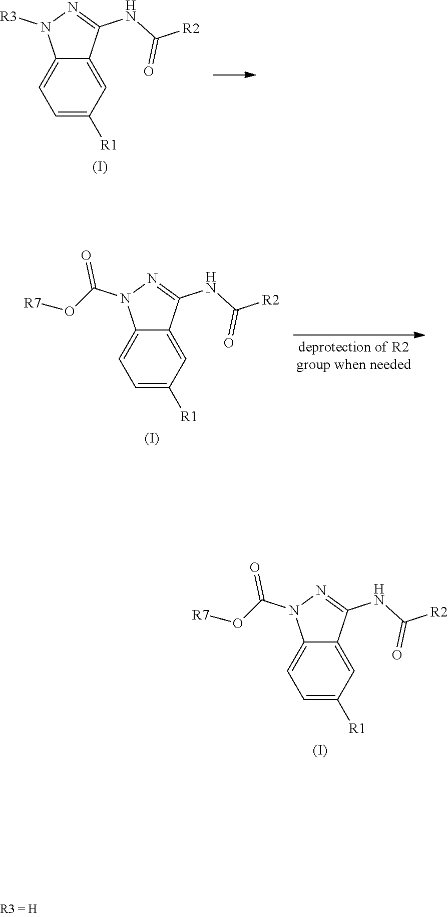

wherein R1 is defined as above in step 1b′ and R2 is an optionally substituted straight or branched (C2-C6) alkyl, (C3-C7) cycloalkyl, heterocyclyl and 5 to 6-membered heterocyclylmethyl wherein the heterocyclyl is selected from the group consisting of pirrolydinyl, and piperidinyl, into a compound of formula (I), by reacting with a compound of formula R7OCOCl (XIX) wherein R7 is an optionally substituted straight or branched (C1-C6) alkyl, (C3-C7) cycloalkyl, aryl, heterocyclyl or heteroaryl, under basic condition;
or
compounds of formula (I) wherein R1 is a group of formula (II) wherein R4 is an optionally substituted straight or branched (C2-C6) alkyl, (C3-C7) cycloalkyl or heterocyclyl, can be prepared as reported in Scheme 2 below:

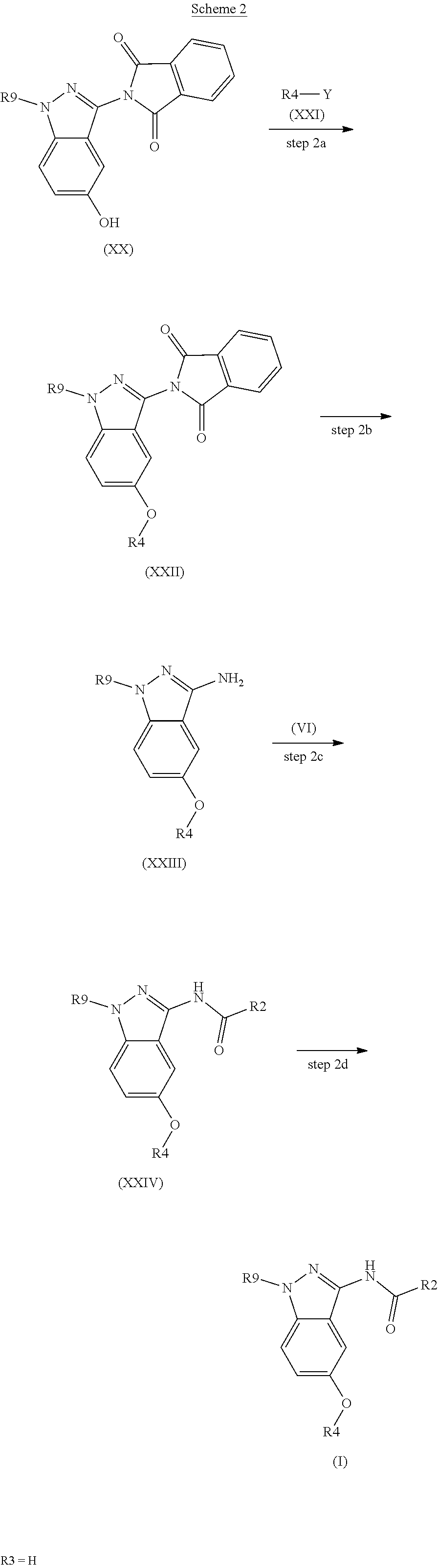

accordingly, the process of the present invention comprises the following steps:
Step 2a) reacting a compound of formula (XX):

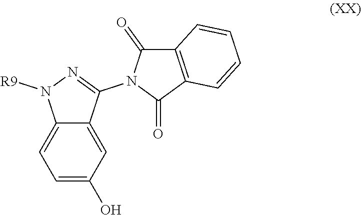

wherein R9 is defined as above in step 1a, with a compound of formula (XXI):

R4-Y (XXI)
wherein R4 an optionally substituted straight or branched (C2-C6) alkyl, (C3-C7) cycloalkyl or heterocyclyl, and Y is halogen or hydroxy;
Step 2b) reacting the obtained intermediate (XXII):

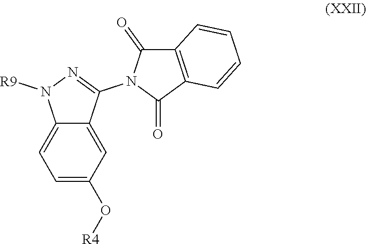

with the suitable deprotecting agent;
Step 2c) mixing the obtained intermediate (XXIII):

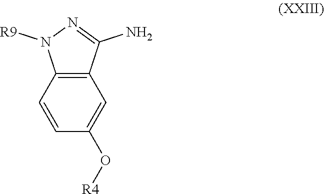

with a compound of formula (VI):

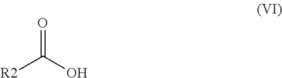

wherein R2 is defined as above in step 1a;
Step 2d) reacting the obtained intermediate (XXIV):

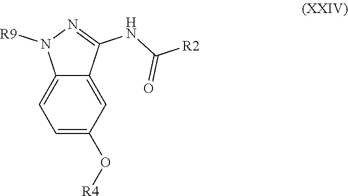

with the suitable deprotecting agent to obtain a compound of formula (I):

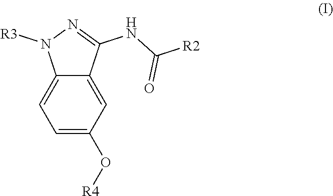

wherein R3 is hydrogen and R2 and R4 are defined as above.

According to step 1a, the coupling reaction of a compound of formula (V), with a suitable carboxylic acid of formula (VI), is carried out under basic conditions, preferably with DIPEA or TEA, in a suitable solvent such as DCM, DMF, THF, 1,4-dioxane or DMA, in the presence of a suitable condensing agent, for instance dicyclohexylcarbodiimide (DCC), 1-ethyl-3-(3′-dimethylaminopropyl)carbodiimide (EDC), 3,4-dihydro-3-hydroxy-4-oxo-1,2,3-benzotriazine (DHBT), 0-benzotriazolytetramethylisouronium tetrafluoroborate (TBTU), benzotriazol-1-yloxytripyrrolidinophosphonium hexafluorophosphate (PyBOP) or 2-(1H-benzotriazole-1yl)-1,1,3,3-tetramethyluronium hexafluorophosphate (HBTU) at a temperature ranging from about −10° C. to reflux and for a suitable time for instance from about 30 minutes to about 96 hours. Said reaction is optionally carried out in the presence of a suitable catalyst such as DMAP, or in presence of a further coupling reagent such as N-hydroxybenzotriazole (HOBT); or
alternatively, conversion can be also carried out, through mixed anhydride method, by using al alkyl chloroformate such as ethyl, isopropyl, benzyl chloroformate, in the presence of a tertiary amine, such as TEA, DIPEA or pyridine, in a suitable solvent such as, for instance, DCM, DMF, THE and the like, at r.t.; or alternatively the carboxylic acid of formula (VI) is converted first into the corresponding acyl chloride by reaction with an activating agent such as thionyl chloride, oxalyl chloride, cyanuric chloride or 1-chloro-N,N,2-trimethylpropenylamine (Ghosez's reagent) neat or in a suitable solvent, such as toluene or DCM, optionally in the presence of a catalytic amount of DMF, at a temperature ranging from about −10° C. to reflux and for a suitable time, for instance, from about 30 minutes to about 5 hours. Then, said acyl chloride is reacted with the suitable amine of formula (V), in a suitable solvent such as DCM, THF, diethyl ether, 1,4-dioxane, ACN, toluene or DMF and the like at a temperature ranging from about −10° C. to reflux and for a suitable time, for instance from about 30 minutes to about 96 hours, in the presence of a suitable base such as TEA, DIPEA or pyridine.

According to step 1b′ and 1b″, reaction of a compound of formula (VII) with a compound of formula (VIII), (IX) or (X), is performed under standard Suzuki coupling conditions using a Pd-based catalyst, such as dichloro[1,1′-bis(diphenylphosphino)ferrocene]palladium(II) dichloromethane adduct, [1,1′-Bis(diphenylphosphino)ferrocene]-dichloropalladium, Tetrakis(triphenylphosphine)-palladium, Tris(dibenzylideneacetone)dipalladium and the like, with a suitable base such as Na₂CO₃, Cs₂CO₃, K₃PO₄, in a suitable solvent such as 1,4-dioxane, 1,4-dioxane/water, THF, DMF, toluene and the like, at a temperature ranging from r.t. to 130° C., in classical thermal conditions, or in microwave apparatus for a time period ranging from 1 hour to 48 hours.

According to step 1c, the removal of R9 protecting group on the compound of formula (XI) can be carried out following procedures which are well known in the art. depending of the protecting group of choice, the following conditions can be employed: for example trityl or tert-butoxycarbonyl (Boc) groups can be removed under acidic conditions such as for instance TFA, HCl and the like in a solvent such as DCM, 1,4-dioxane, at a temperature ranging from r.t. to reflux, for a suitable time, for instance from about 1 hour to 48 hours; methoxy or ethoxycarbonyl groups can be removed under basic conditions such for instance TEA, DIPEA, Na₂CO₃, K₂CO₃ and the like in a solvent such as MeOH, EtOH, at a temperature ranging from r.t to reflux, for a suitable time, for instance from 30 minutes to 24 hours.

According to step 1d, the Suzuki reaction of compound of formula (V) with compound of formula (VIII) or (IX) is performed as described as for step. 1b.

According to step 1e, the amidation of compound of formula (XII) with a carboxylic acid of formula (VI) can be carried out as described as for step 1a.

According to step 1f, the deprotection of a compound of formula (XIII) is performed as described as for step 1c.

According to the conv. A of the process, at first deprotection of a compound of formula (VIIa) is performed, depending of the protecting group of choice, under acidic, basic or hydrogenation conditions to provide the free amine of formula (VIIb). Then, this intermediate is further reacted in reductive amination conditions with a reagent of formula (XIV), in the presence of a reductive agent such as NaBH₄, NaCNBH₃, NaBH(OAc)₃ and the like, in a solvent such as MeOH, EtOH, DMF and the like, at a temperature ranging from r.t. to 40° C. and for a time ranging from about 1 hour to 12 hours. Said reducing reaction can be optionally carried out in the presence of a suitable catalyst such as AcOH, TFA and the like.

According to the conv. B, the reductive amination of a compound of formula (VIId) with an amine of formula (XV) is performed as described as for conv. A.

According to the conv. C, the reactions on a compound of formula (XIIIa) to yield a compound of formula (XIIc) are performed as described as for conv. A.

According to the conv. D, the reductive amination of a compound of formula (XIIId) with an amine of formula (XV) is performed as described as for conv. B.

According to the conv. E of the process, at first the reaction of the piperidine ring of a compound of formula (XIIIf) with a chloroformate of a formula (XVI) is carried out under basic condition, preferably with DIPEA, TEA and pyridine, in a suitable solvent such as DCM, DMF, THF, 1,4-dioxane or DMA, at a temperature ranging from about −10° C. to 50° C. and for a suitable time for instance from about 30 minutes to about 24 hours. Then intermediate (XIIIg) is further reacted with a carboxylic acid of formula (XVII), with a suitable base such as Na₂CO₃, K₂CO₃, Cs₂CO₃, and the like, in a suitable solvent such as 1,4-dioxane, 1,4-dioxane/water, THF, DMF, toluene and the like, at a temperature ranging from r.t. to 60° C., and for a suitable time, for instance, from about 2 hours to about 96 hours. Said reaction is optionally carried out in the presence of a suitable catalyst such as Nal or Kl.

According to the conv. 1, the reactions of a compound of formula (I) with a compound of formula (XIX) are carried out under basic condition, preferably with DIPEA, TEA and pyridine, in a suitable solvent such as DCM, DMF, THF, 1,4-dioxane or DMA, at a temperature ranging from about −10° C. to 50° C. and for a suitable time for instance from about 30 minutes to about 24 hours.

According to step 2a, the alkylation of an intermediate of formula (XX) with an intermediate of formula (XXI), wherein Y is bromine or iodine, can be carried out in the presence of the suitable base such as Na₂CO₃, K₂CO₃, Cs₂CO₃, NaH, KH and the like, in a suitable solvent, such as DMF, DMA, ACN, acetone, THF and the like, at a temperature ranging from 0° C. to reflux in a period time varying from 1 hour to 24 hours. When is used an intermediate of formula (XXI) wherein Y is hydroxy, the reaction is preferentially curried out under Mitsunobu alkylation conditions in presence of a suitable reagent such us, for instance, diethylazodicarboxylate (DEAD), diisopropylazodicarboxylate (DIAD), ditertbutylazodicarboxylate (DBAD), 1,1′-(azodicarbonyl)dipiperidine (ADDP), and a phosphine reagent such as, for instance, trimethylphosphine, tritertbutylphosphine and the like, in a suitable solvent, such as THF, DMF, DCM, toluene, benzene and the like, at a temperature ranging from 0° C. to 65° C.

According to step 2b, the deprotection of a compound of formula (XXII) is performed with hydrazine, in a suitable solvent, such as MeOH, EtOH and the like, at a temperature ranging from r.t. to reflux in a period time varying from about 1 hour to 96 hours.

According to step 2c, the amidation of a compound of formula (XXIII) with a carboxylic acid of formula (VI) can be carried out as described as for step 1a.

According to step 2d, the deprotection of a compound of formula (XXIV) is performed as described as for step 1c.

From all of the above, it is clear to the skilled person that the conversion of a compound of formula (I) into a pharmaceutically acceptable salt thereof or, alternatively, the conversion into the free compound (I) of a corresponding salt, according to procedures well-known in the art, is still within the scope of the invention. When preparing the compounds of formula (I) according to any variant of the process, which are all to be intended as within the scope of the invention, optional functional groups within the starting materials, the reagents or the intermediates thereof, and which could give rise to unwanted side reactions, need to be properly protected according to conventional techniques.

Protection of such reactive centers, and subsequent deprotection at the end of the synthetic transformations, can be accomplished following standard procedures described, for instance, in: Green, Theodora W. and Wuts, Peter G. M.—Protective Groups in Organic Synthesis, Third Edition, John Wiley & Sons Inc., New York (NY), 1999.

According to any variant of the process for preparing the compounds of the formula (I), the starting materials and any other reactants are known or easily prepared according to known methods or commercially available.

The final compounds may be isolated and purified using conventional procedures, for example chromatography and/or crystallization and salt formation.

The compounds of general formula (I) as defined above can be converted into pharmaceutically acceptable salts.

The synthesis of a compound of general formula (I), according to the synthetic processes described above, can be conducted in a stepwise manner, whereby each intermediate is isolated and purified if needed by standard purification techniques, like, for example, column chromatography, before carrying out the subsequent reaction. Alternatively, two or more steps of the synthetic sequence can be carried out in a so-called “one-pot” procedure, as known in the art, whereby only the compound resultant from the two or more steps is isolated and purified. In cases where a compound of general formula (I) contains one or more asymmetric centers, said compound can be separated into the single stereoisomers by procedures known to those skilled in the art. Such procedures comprise standard chromatographic techniques, including chromatography using a chiral stationary phase, or crystallization. General methods for separation of compounds containing one or more asymmetric centers are reported, for instance, in Jacques, Jean; Collet, Andr6; Wilen, Samuel H., Enantiomers, Racemates, and Resolutions, John Wiley & Sons Inc., New York (NY), 1981.

The present invention also provides a method for treating diseases caused by and/or associated with dysregulated protein kinase activity, particularly CDK1/CYCB, CDK2/CYCA, CDK4/CYCD1, CDK7/CYCH/MAT1, CDK9/CYCT, CDK11A, CDK11B, more particularly CDK11 family kinases, which comprises administering to a mammal in need thereof, more particularly a human, an effective amount of compound of formula (I) as defined above.

Furthermore the present invention provides a compound of formula (I) or a pharmaceutically acceptable salt thereof, as defined above, for use in a method of treating a desease caused by and/or associated with dysregulated protein kinase activity reported above, particularly CDK11 kinase activity, which comprises administering to a mammal, preferably a human, in need thereof, an effective amount of a compound of formula (I) as defined above.

A preferred method of the present invention is to treat a disease caused by and/or associated with dysregulated protein kinase activity selected from the group consisting of cancer, cell proliferative disorders, viral infections, immune disorders, neurodegenerative disorders and cardiovascular diseases. More preferably, the disease is cancer.

According to a most preferred embodiment of the present invention the cancer is selected from the group consisting of: carcinomas, such as bladder, breast, kidney, liver, colon, lung, including small cell lung cancer, esophagus, gall-bladder, ovary, pancreas, stomach, cervix, prostate, and skin, including squamous cell carcinoma; hematopoietic tumors of lymphoid lineage including leukemia, acute lymphocitic leukemia, acute lymphoblastic leukemia, chronic lymphocytic leukemia, B-cell lymphoma, angioimmunoblastic T-cell lymphoma, Hodgkin's lymphoma, non-Hodgkin's lymphoma, hairy cell lymphoma and Burkitt's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including glioma, glioblastoma, glioblastoma multiforme, astrocytoma, oligodendroglioma, paraglioma, neuroblastoma, and schwannomas; and other tumors, including melanoma, seminoma, teratocarcinoma, osteosarcoma, xeroderma pigmentosum, keratoxanthoma, thyroid cancers, such as papillary thyroid carcinoma and medullary thyroid carcinoma, Kaposi's sarcoma, chondrosarcoma, and cholangiocarcinoma.

Another preferred method of the present invention is to treat specific cellular proliferation disorders such as, for example, benign prostate hyperplasia, familial adenomatosis polyposis, neurofibromatosis, psoriasis, vascular smooth cell proliferation associated with atherosclerosis, pulmonary fibrosis, arthritis, glomerulonephritis and postsurgical stenosis and restenosis.

Another preferred method of the present invention is to treat viral infections, in particular the prevention of AIDS development in HIV-infected individuals.

Another preferred method of the present invention is to treat immune disorders, such as inflammatory and autoimmune diseases, for examples multiple sclerosis, rheumatoid arthritis (RA), systemic lupus erythematous, inflammatory bowel diseases (IBD), Crohn's disease, irritable bowel syndrome, pancreatitis, ulcerative colitis, diverticulosis, myasthenia gravis, vasculitis, psoriasis, scleroderma, asthma, allergy, systemic sclerosis, vitiligo, arthritis such as osteoarthritis, juvenile rheumatoid arthritis, ankylosing spondylitis.

Another preferred method of the present invention is to treat neurodegenerative disorders, such as Alzheimer's disease, Parkinson's disease and Huntington's disease.

Another preferred method of the present invention is to treat specific cardiovascular diseases, such as coronary heart diseases, cardiomyopathies, ischaemic heart diseases, heart failure, hypertensive heart diseases, inflammatory heart diseases and valvular heart diseases.

In addition, the method of the present invention also provides tumor angiogenesis and metastasis inhibition as well as the treatment of organ transplant rejection and host versus graft disease.

The present invention further provides a pharmaceutical composition comprising a compound of formula (I) in combination with one or more chemotherapeutic—e.g. cytostatic or cytotoxic agents. Cytostatic or cytotoxic agents include, but are not limited to antibiotic-type agents, alkylating agents, antimetabolite agents, hormonal agents, immunological agents, interferon-type agents, cyclooxygenase inhibitors (e.g. COX-2 inhibitors), matrixmetalloprotease inhibitors, telomerase inhibitors, tyrosine kinase inhibitors, anti-growth factor receptor agents, anti-HER agents, anti-EGFR agents, anti-angiogenesis agents (e.g. angiogenesis inhibitors), farnesyl transferase inhibitors, ras-raf signal transduction pathway inhibitors, cell cycle inhibitors, other cdks inhibitors, tubulin binding agents, topoisomerase I inhibitors, topoisomerase II inhibitors aromatase inhibitors, inhibitors of kinesins, therapeutic monoclonal antibodies, inhibitors of mTOR, histone deacetylase inhibitors, inhibitors of hypoxic response, PD-1 antagonists, or antigen binding fragment thereof, which specifically binds to PD-1 or PD-L1 and the like.

If formulated as a fixed dose, such combination products employ the compounds of this invention within the dosage range described below and the other pharmaceutically active agent within the approved dosage range.

The present invention further provides an in vitro method for inhibiting CDK11 protein kinase activity which comprises contacting the CDK11 kinase with an effective amount of a compound of formula (I) as defined above. Additionally, the invention provides a product comprising a compound of formula (I) or a pharmaceutically acceptable salt thereof, as defined above, and one or more chemotherapeutic agents, as a combined preparation for simultaneous, separate or sequential use in anticancer therapy.

The compounds of formula (I) of the present invention, suitable for administration to a mammal, e.g. to humans, can be administered by the usual routes and the dosage level depends upon the age, weight, and conditions of the patient and administration route.

For example, a suitable dosage adopted for oral administration of a compound of formula (I) may range from about 10 to about 1000 mg per dose, from 1 to 5 times daily. The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or through intravenous and/or intrathecal and/or intraspinal injection or infusion.

The pharmaceutical compositions containing the compounds of the invention are usually prepared following conventional methods and are administered in a suitable pharmaceutical form.

For example, the solid oral forms may contain, together with the active compound, diluents, e.g. lactose, dextrose, saccharose, sucrose, cellulose, corn starch or potato starch; lubricants, e.g. silica, talc, stearic acid, magnesium or calcium stearate, and/or polyethylene glycols; binding agents, e.g. starches, arabic gum, gelatine methylcellulose, carboxymethylcellulose or polyvinyl pyrrolidone; disintegrating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. These pharmaceutical preparations may be manufactured in known manner, for example, by means of mixing, granulating, tabletting, sugar-coating, or film-coating processes.

The suspensions and the emulsions may contain, as examples of carriers, natural gum, agar, sodium alginate, pectin, methylcellulose, carboxymethylcellulose or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol and, if desired, a suitable amount of lidocaine hydrochloride.

The solutions for intravenous injections or infusions may contain, as a carrier, sterile water or preferably they may be in the form of sterile, aqueous, isotonic, saline solutions or they may contain propylene glycol as a carrier.

The suppositories may contain, together with the active compound, a pharmaceutically acceptable carrier, e.g. cocoa butter, polyethylene glycol, a polyoxyethylene sorbitan fatty acid ester surfactant or lecithin.

In another aspect the invention provides a compound of formula (I) or a pharmaceutically acceptable salt thereof, as defined above, for use as a medicament.

Finally, the invention provides the use of a compound of formula (I) or a pharmaceutically acceptable salt thereof, as defined above, in the manufacture of a medicament with anticancer activity.

### Experimental Section

The short forms and abbreviations used herein, as well as throughout the description, have the following meaning:

ABBREVIATIONS

| | |
|---|---|
| EtOAc | Ethyl acetate |
| Bz | Benzyl |
| DCM | Dichloromethane |
| DIPEA | N,N-Diisopropyethylamine |
| DMA | N,N-Dimethylacetamide |
| DMF | N,N-Dimethylformamide |
| DMSO | Dimethyl sulfoxide |
| Et₂O | Diethyl ether |
| EtOH | Ethanol |
| iPrOH | Isopropanol |
| MeOH | Methanol |
| MTBE | Methyl tert-butyl ether |
| PdCl₂(dppf) | 1,1′-bis(Diphenylphosphino)ferrocene]palladium(II) |
| | chloride |
| TEA | Triethylamine |
| THF | Tetrahydrofuran |
| h | Hour/s |
| min | Minute/s |
| r.t. | Room temperature |
| rt | Retention time |

### Preparation of Compounds of Formula (I)

For a reference to any specific compound of formula (I) of the invention, optionally in the form of a pharmaceutically acceptable salt, see the experimental section and claims. Referring to the examples that follow, compounds of the present invention were synthesized using the methods described herein, or other methods, which are well known in the art.

With the aim at better illustrating the present invention, without posing any limitation to it, the following examples are given.

As used herein the symbols and conventions used in the processes, schemes and examples are consistent with those used in the contemporary scientific literature, for example, the Journal of the American Chemical Society or the Journal of Biological Chemistry.

Compound names are IUPAC names, generated by using ACD Name (by Advanced Chemistry Development, Inc.). Unless otherwise noted, all materials, including anhydrous solvent such as DMF, THF, DCM, were obtained from commercial suppliers, of the best grade and used without further purification. All reactions involving air- or moisture-sensitive compounds were performed under nitrogen or argon atmosphere.

### General Purification and Analytical Methods

The synthetic preparation of some compounds of formula (I) of the invention is described in the following examples.

The compounds of the present invention, as prepared according to the following examples, were also characterized by ¹H NMR and/or by HPLC/MS analytical data; HPLC/MS data were collected following any one of methods LCQ or LCT. Flash Chromatography was performed on silica gel (Merck grade 9395, 60A).

### HPLC LCQ Method

HPLC-MS/UV analyses were performed on a LCQ DecaXP (Thermo, San Jose, US) ion trap instrument, equipped with an electrospray (ESI) ion source. The mass spectrometer is connected to a Surveyor HPLC system (Thermo, San Jose, US) with an UV photodiode array detector (UV detection 215-400 nm). A Waters XSelect CSH C18 column 50×4.6 mm, 3.5 m particle size was used. Mobile phase A was ammonium acetate 5 mM buffer (pH 4.5 with acetic acid):acetonitrile 95:5, and mobile phase B was ammonium acetate 5 mM buffer (pH 4.5 with acetic acid): acetonitrile 5:95. Gradient from 0 to 100% B in 7 minutes, hold 100% B 2 minutes. Flow rate 1 mL/min. Injection volume 10 μL. Retention times (HPLC r.t.) are given in minutes. Full scan, mass range from 50 to 1200 amu. Heated capillary temp was 200° C. and Spray voltage value was set at 4 kV. Mass are given as m/z ratio. Instrument control, data acquisition and processing were performed by using Xcalibur 1.4 SR1 software (Thermo).

### HPLC LCT Method

HPLC-MS/UV analyses and High Resolution Mass Spectra (HRMS) were performed on a A Waters Alliance LC 2795 equipped with a Waters PDA UV detector 2996 and a TOF Waters LCT Premier XE mass detector (ESI interface) supported by a Waters Reagent Manager liquid pump. The assay is based on generic gradient reversed-phase chromatography that allows complementing an identity-purity assay with determination and confirmation of the expected exact mass of the compounds in the same run. Compound identity is accomplished by on-line serial ESI(+) Full Scan MS detection, sample purity is obtained as relative “Area Percent” of the integrated LC/UV trace at 216-400 nm. The liquid chromatograph is equipped with a Waters XBridge CSH C18 column (3.0×30 mm, 3.5 m particle size) thermostated at 50° C. Alternatively a Supelco column Ascentis Express C18 (2.7×30 mm×3 um) was used.

Mobile phases A was 0.05% w/v formic acid in highly purified water and mobile phase B was a 70/25/5 (v/v/v) mixture of MeOH/iPrOH/H2O containing 0.035% w/v of formic acid. Gradient from 0 to 100% B in 17.5 minutes, hold 100% B 5 minutes. Flow rate 0.8 mL/min, Injection volume 4 μL. The ESI source operated at 100° C., 2.5 kV capillary voltage, 60 V cone, 600 L/hr nitrogen desolvation flow at 350° C. and 10 L/hr nitrogen cone flow. The “Normal” Zfocus is set at 140. The analyzer is normally optimized at 7200 V flight tube.

In order to obtain high resolution mass spectra, the eluent from the HPLC column was split and 25 μL/min were mixed with a 100 μL/min stream of a 30/10/60 (v/v/v) mixture of MeOH/iPrOH/H2O containing 0.01% w/v of formic acid and 80 nM Trimethoprim coming from a Waters Reagent Manager pump before entering the MS source.

Trimethoprim was chosen as stable, soluble and appropriate reference compound for real-time single-point mass correction. ES(+) full scan 80-1200 amu centroided data acquisition was carried out at 2 Hz sampling rate in the “W” mode. The LCT embedded PC provided both real-time data centroiding and real-time mass correction based on the Trimethoprim. H+ reference mass of 291.1452 Da. Proper intensity MS spectra (40 to 2000 analyte counts) were averaged to obtain the final result.

¹H-NMR spectra were recorded at a constant temperature of 28° C. on a Varian INOVA 400 spectrometer operating at 400.5 MHz and equipped with a 5 mm ¹H{¹⁵N-³¹P}z-axis PFG Indirect Detection probe and on a Varian INOVA 500 spectrometer operating at 499.7 MHz and equipped with a 5 mm ¹H{¹³C-¹⁵N}triple resonance Indirect Detection probe. Chemical shifts were referenced with non deuterated residual solvent signal (DMSO-d₅: 2.50 ppm for ¹H). Data are reported as follows: chemical shift (6), multiplicity (s=singlet, d=doublet, t=triplet, q=quartet, qt=quintet, br. s=broad singlet, dd=doublet of doublets, ddd=doublet of doublets of doublets, m=multiplet), coupling constants (J, Hz) and number of protons.

### Example 1

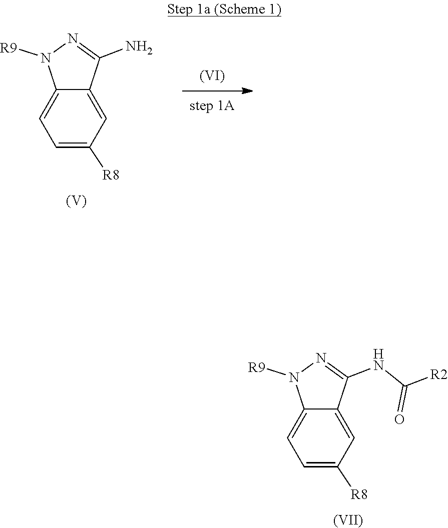

### tert-Butyl 3-[(5-bromo-1-trityl-1H-indazol-3-yl)carbamoyl]piperidine-1-carboxylate[(VII), R2=tert-butyl piperidine-1-carboxylate, R8=Br, R9=1,1′, 1″-methanetriyltribenzene]

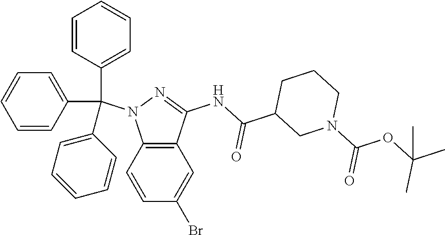 [SMILES: CC(C)(C)OC(=O)N1CCCC(C(=O)Nc2nn(C(c3ccccc3)(c3ccccc3)c3ccccc3)c3ccc(Br)cc23)C1]

To a solution of 1-(tert-butoxycarbonyl)piperidine-3-carboxylic acid (0.093 g, 0.405 mmol) in dry DCM (5 mL), cooled at 0-5° C., 1-chloro-N,N,2-trimethyl-1-propenylamine (Ghosez's reagent) (0.080 mL, 0.608 mmol) was added. The mixture was stirred at room temperature for 2 h, then was added dropwise to a solution of 5-bromo-1-trityl-1H-indazol-3-ylamine (0.153 g, 0.338 mmol) and pyridine (0.098 mL, 1.215 mmol) in dry THF (10 mL), cooled at 0-5° C. The reaction was stirred at room temperature for 2 h, then diluted with DCM (20 mL) and washed with 10% aqueous citric acid (20 mL), water (20 mL) and brine (20 mL). The organic phase was dried with anhydrous Na₂SO₄, filtered and evaporated to dryness. The crude was purified by flash chromatography (Hexane/EtOAc 70:30) to obtain tert-butyl 3-[(5-bromo-1-trityl-1H-indazol-3-yl)carbamoyl]piperidine-1-carboxylate (0.215 g, 96% of yield) as white solid.

¹H NMR (500 MHz, DMSO-d₆) δ ppm 1.27-1.47 (m, 9H), 1.60-1.76 (m, 2H), 1.95 (d, J=11.90 Hz, 1H), 2.54-2.61 (m, 1H), 2.82 (m, 1H), 2.88-3.19 (m, 1H), 3.80 (d, J=12.35 Hz, 1H), 4.03 (q, J=7.17 Hz, 2H), 6.33 (d, J=9.15 Hz, 1H), 7.17-71.18 (m, 7H), 7.26-7.30 (m, 3H), 7.30-7.36 (m, 6H), 7.93 (d, J=1.53 Hz, 1H), 10.67 (s, 1H). HRMS (ESI) calcd for C37H38BrN4O3Na [M+H+Na]⁺ 687.1941, found 687.1938.

According to this same methodology, but employing suitable intermediates and reagents, the following compounds were prepared:

### (R)-tert-Butyl 3-[(5-bromo-1-trityl-1H-indazol-3-yl)carbamoyl]piperidine-1-carboxylate [(VII), R2=tert-butyl piperidine-1-carboxylate, R8=Br, R9=1,1′,1″-methanetriyltribenzene]

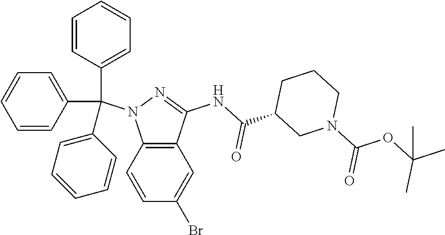 [SMILES: CC(C)(C)OC(=O)N1CCC[C@@H](C(=O)Nc2nn(C(c3ccccc3)(c3ccccc3)c3ccccc3)c3ccc(Br)cc23)C1]

Starting from 5-bromo-1-trityl-1H-indazol-3-ylamine and (3R)-1-(tert-butoxycarbonyl)piperidine-3-carboxylic acid. Flash column chromatography (Hexane/EtOAc 70:30). Obtained 0.150 g (90% of yield). 1H NMR (500 MHz, DMSO-d₆) δ ppm 1.27-1.47 (m, 9H), 1.60-1.76 (m, 2H), 1.95 (d, J=11.90 Hz, 1H), 2.54-2.61 (m, 1H), 2.82 (m, 1H), 2.88-3.19 (m, 1H), 3.80 (d, J=12.35 Hz, 1H), 4.03 (q, J=7.17 Hz, 2H), 6.33 (d, J=9.15 Hz, 1H), 7.17-71.18 (m, 7H), 7.26-7.30 (m, 3H), 7.30-7.36 (m, 6H), 7.93 (d, J=1.53 Hz, 1H), 10.67 (s, 1H). HRMS (ESI) calcd for C37H38BrN4O3Na [M+H+Na]⁺687.1941, found 687.1935.

### (S)-tert-Butyl 3-[(5-bromo-1-trityl-1H-indazol-3-yl)carbamoyl]piperidine-1-carboxylate [(VII), R2=tert-butyl piperidine-1-carboxylate, R8=Br, R9=1,1′, 1″-methanetriyltribenzene]

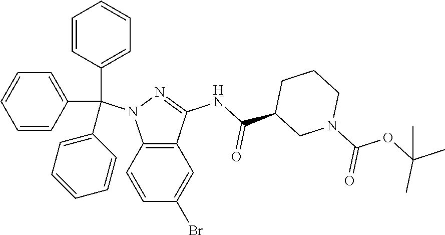 [SMILES: CC(C)(C)OC(=O)N1CCC[C@H](C(=O)Nc2nn(C(c3ccccc3)(c3ccccc3)c3ccccc3)c3ccc(Br)cc23)C1]

Starting from 5-bromo-1-trityl-1H-indazol-3-ylamine and (3S)-1-(tert-butoxycarbonyl)piperidine-3-carboxylic acid. Flash column chromatography (Hexane/EtOAc 70:30). Obtained 0.110 g (93% of yield). 1H NMR (500 MHz, DMSO-d₆) δ ppm 1.27-1.47 (m, 9H), 1.60-1.76 (m, 2H), 1.95 (d, J=11.90 Hz, 1H), 2.54-2.61 (m, 1H), 2.82 (m, 1H), 2.88-3.19 (m, 1H), 3.80 (d, J=12.35 Hz, 1H), 4.03 (q, J=7.17 Hz, 2H), 6.33 (d, J=9.15 Hz, 1H), 7.17-71.18 (m, 7H), 7.26-7.30 (m, 3H), 7.30-7.36 (m, 6H), 7.93 (d, J=1.53 Hz, 1H), 10.67 (s, 1H). HRMS (ESI) calcd for C37H38BrN4O3Na [M+H+Na]⁺687.1941, found 687.1943.

### N-(5-Bromo-1-trityl-1H-indazol-3-yl)-1-methylpiperidine-3-carboxamide [(VII), R2=methylpiperidine, R8=Br, R9=1,1′, 1″-methanetriyltribenzene]

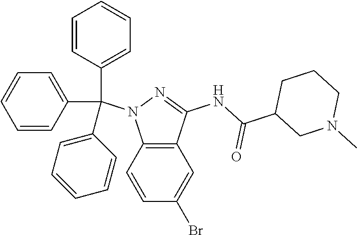 [SMILES: CN1CCCC(C(=O)Nc2nn(C(c3ccccc3)(c3ccccc3)c3ccccc3)c3ccc(Br)cc23)C1]

Starting from 5-bromo-1-trityl-1H-indazol-3-ylamine and 1-methylpiperidine-3-carboxylic acid. Flash column chromatography (DCM/MeOH/33% aqueous ammonia 95:5:0.5). Obtained 0.293 g (67% of yield). ¹H NMR (500 MHz, DMSO-d₆) δ ppm 1.34-1.55 (m, 2H), 1.66 (d, J=12.35 Hz, 1H), 1.84 (d, J=10.68 Hz, 2H), 1.98-2.07 (m, 1H), 2.17 (s, 3H), 2.66-2.75 (m, 2H), 2.86 (d, J=10.68 Hz, 1H), 6.32 (d, J=9.15 Hz, 1H), 7.14-7.21 (m, 7H), 7.25-7.30 (m, 3H), 7.31-7.36 (m, 6H), 7.89 (d, J=1.22 Hz, 1H), 10.58 (s, 1H). HRMS (ESI) calcd for C33H32BrN4O [M+H]⁺ 579.1754, found 579.1766.

### tert-Butyl 4-[(5-bromo-1-trityl-1H-indazol-3-yl)carbamoyl]piperidine-1-carboxylate [(VII), R2=tert-butyl piperidine-1-carboxylate, R8=Br, R9=1,1′, 1″-methanetriyltribenzene]

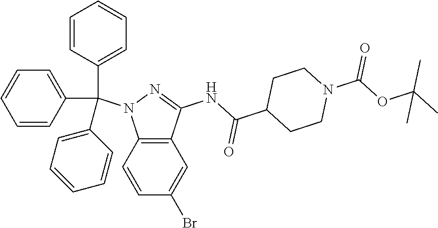 [SMILES: CC(C)(C)OC(=O)N1CCC(C(=O)Nc2nn(C(c3ccccc3)(c3ccccc3)c3ccccc3)c3ccc(Br)cc23)CC1]

Starting from 5-bromo-1-trityl-1H-indazol-3-ylamine and 1-(tert-butoxycarbonyl)piperidine-4-carboxylic acid. Flash column chromatography (Hexane/EtOAc 70:30). Obtained 0.100 g (90% of yield). LCMS (ESI) m/z 666 (M+H)⁺.

### tert-Butyl (3R)-3-{[5-(4,4,5,5-tetramethyl-1,3,2-dioxaborolan-2-yl)-1-trityl-1H-indazol-3-yl]carbamoyl}piperidine-1-carboxylate [(VII), R2=tert-butyl piperidine-1-carboxylate, R8=4,4,5,5-tetramethyl-1,3,2-dioxaborolan-2-yl, R9=1,1′, 1″-methanetriyltribenzene]

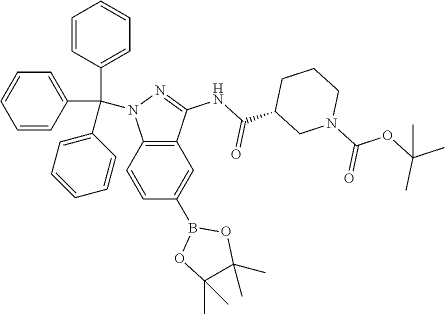 [SMILES: CC(C)(C)OC(=O)N1CCC[C@@H](C(=O)Nc2nn(C(c3ccccc3)(c3ccccc3)c3ccccc3)c3ccc(B4OC(C)(C)C(C)(C)O4)cc23)C1]

Starting from 5-(4,4,5,5-tetramethyl-1,3,2-dioxaborolan-2-yl)-1-trityl-1H-indazol-3-amine and (3R)-1-(tert-butoxycarbonyl)piperidine-3-carboxylic acid. Flash column chromatography (Hexane/EtOAc 70:30). Obtained 0.077 g (92% of yield). ¹H NMR (500 MHz, DMSO-d₆) δ ppm 1.26 (s, 12H), 1.31-1.39 (m, 10H), 1.59-1.78 (m, 2H), 1.96-2.00 (m, 1H), 2.61 (m, 1H), 2.73-2.82 (m, 2H), 3.87 (d, J=12.96 Hz, 1H), 3.96-4.05 (m, 1H), 6.39 (d, J=8.69 Hz, 1H), 7.17 (d, J=7.47 Hz, 6H), 7.24-7.28 (m, 4H), 7.30-7.33 (m, 6H), 8.16 (s, 1H), 10.59 (br. s., 1H). HRMS (ESI) calcd for C43H50BN405 [M+H]⁺ 712.3905, found 712.3895.

### tert-Butyl (3R)-3-{[5-(4,4,5,5-tetramethyl-1,3,2-dioxaborolan-2-yl)-1-trityl-1H-indazol-3-yl]carbamoyl}-pyrrolidine-1-carboxylate [(XXIX), R2=tert-butyl pyrrolidine-1-carboxylate, R8=4,4,5,5-tetramethyl-1,3,2-dioxaborolan-2-yl, R9=1,1′, 1″-methanetriyltribenzene]

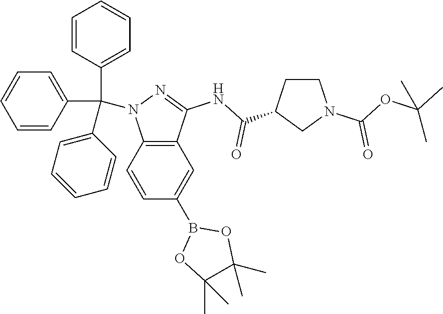 [SMILES: CC(C)(C)OC(=O)N1CC[C@@H](C(=O)Nc2nn(C(c3ccccc3)(c3ccccc3)c3ccccc3)c3ccc(B4OC(C)(C)C(C)(C)O4)cc23)C1]

Starting from 5-(4,4,5,5-tetramethyl-1,3,2-dioxaborolan-2-yl)-1-trityl-1H-indazol-3-amine and (3R)-1-(tert-butoxycarbonyl)pyrrolidine-3-carboxylic acid. Flash column chromatography (Hexane/EtOAc 70:30). Obtained 0.127 g (89% of yield). 1H NMR (500 MHz, DMSO-d₆) δ ppm 1.26 (s, 12H), 1.41 (s, 9H), 2.00-2.20 (m, 2H), 3.15-3.30 (m, 2H), 3.38-3.45 (m, 2H), 3.55 (m, 1H), 6.40 (d, J=8.85 Hz, 1H), 7.17 (d, J=7.63 Hz, 6H), 7.25-7.29 (m, 4H), 7.30-7.33 (m, 6H), 8.15 (br. s., 1H), 10.66 (d, J=6.41 Hz, 1H). HRMS (ESI) calcd for C42H48BN4O5 [M+H]⁺ 698.3749, found 698.3740.

### N-(5-Bromo-1-trityl-1H-indazol-3-yl)-6-oxopiperidine-3-carboxamide [(VII), R2=piperidin-2-one, R8=Br, R9=1,1′, 1″-methanetriyltribenzene]

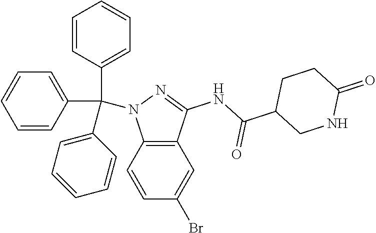 [SMILES: O=C1CCC(C(=O)Nc2nn(C(c3ccccc3)(c3ccccc3)c3ccccc3)c3ccc(Br)cc23)CN1]

Starting from 5-bromo-1-trityl-1H-indazol-3-ylamine and 6-oxopiperidine-3-carboxylic acid. Flash column chromatography (DCM/MeOH 95:5). Obtained 0.157 g (96% of yield). ¹H NMR (500 MHz, DMSO-d₆) δ ppm 1.85-1.96 (m, 1H), 1.99-2.08 (m, 1H), 2.13-2.30 (m, 2H), 2.51-2.54 (m, 2H), 2.89 (m, 1H), 6.33 (d, J=9.15 Hz, 1H), 7.13-7.23 (m, 7H), 7.26-7.30 (m, 3H), 7.31-7.36 (m, 6H), 7.52 (br. s., 1H), 7.93 (d, J=1.22 Hz, 1H), 10.73 (s, 1H). HRMS (ESI) calcd for C32H28BrN4O2Na [M+H+Na]⁺601.1209, found 601.1234.

### tert-Butyl (3R)-3-[(5-bromo-1-trityl-1H-indazol-3-yl)carbamoyl]pyrrolidine-1-carboxylate [(VII), R2=tert-butyl pyrrolidine-1-carboxylate, R8=Br, R9=1,1′,1″-methanetriyltribenzene]

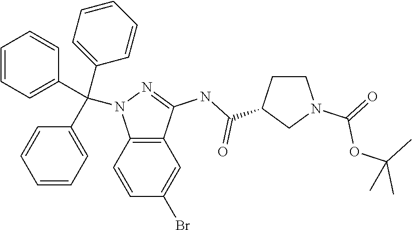 [SMILES: CC(C)(C)OC(=O)N1CC[C@@H](C(=O)Nc2nn(C(c3ccccc3)(c3ccccc3)c3ccccc3)c3ccc(Br)cc23)C1]

Starting from 5-bromo-1-trityl-1H-indazol-3-ylamine and (3R)-1-(tert-butoxycarbonyl)pyrrolidine-3-carboxylic acid. Flash column chromatography (Hexane/EtOAc 70:30). Obtained 0.162 g (97% of yield). 1H NMR (500 MHz, DMSO-d₆) δ ppm 1.40 (s, 9H), 2.00-2.18 (m, 2H), 3.16-3.29 (m, 2H), 3.38-3.45 (m, 2H), 3.52 (m, 1H), 6.34 (d, J=9.15 Hz, 1H), 7.17-7.19 (m, 7H), 7.25-7.31 (m, 3H), 7.31-7.38 (m, 6H), 7.94 (br. s., 1H), 10.73 (br. s., 1H). HRMS (ESI) calcd for C36H36BrN4O3Na [M+H+Na]⁺673.1785, found 673.1804.

### 9H-Fluoren-9-ylmethyl 4-[(5-bromo-1-trityl-1H-indazol-3-yl)carbamoyl]piperidine-1-carboxylate [(VII), R2=piperidine-1,4-dicarboxylic acid mono-(9H-fluoren-9-ylmethyl) ester, R8=Br, R9=methanetriyltribenzene]

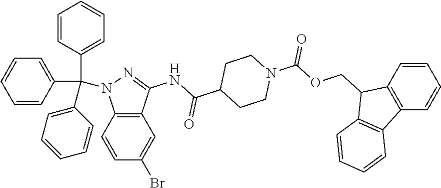 [SMILES: O=C(Nc1nn(C(c2ccccc2)(c2ccccc2)c2ccccc2)c2ccc(Br)cc12)C1CCN(C(=O)OCC2c3ccccc3-c3ccccc32)CC1]

Starting from 5-bromo-1-trityl-1H-indazol-3-ylamine and piperidine-1,4-dicarboxylic acid mono-(9H-fluoren-9-ylmethyl) ester. Flash column chromatography (Hexane/EtOAc 60:40). Obtained 6.53 g (75% of yield). 1H NMR (500 MHz, DMSO-d6) δ ppm 1.40-1.52 (m, 2H), 1.72-1.85 (m, 2H), 2.77-2.82 (m, 1H), 3.46-3.56 (m, 2H), 4.28-4.43 (m, 4H), 6.33 (d, J=8.69 Hz, 1H), 7.16-7.21 (m, J=7.17 Hz, 7H), 7.25-7.37 (m, 11H), 7.40-7.44 (m, 2H), 7.64 (d, J=7.17 Hz, 2H), 7.87-7.95 (m, 3H), 10.59 (s, 1H). HRMS (ESI) calcd for C47H40BrN4O3 [M+H]⁺ 809.2098, found 809.2077.

### N-(5-Bromo-1-trityl-1H-indazol-3-yl)-3-oxocyclobutanecarboxamide [(VII), R2=3-oxocyclobutane, R8=Br, R9=1,1′, 1″-methanetriyltribenzene]

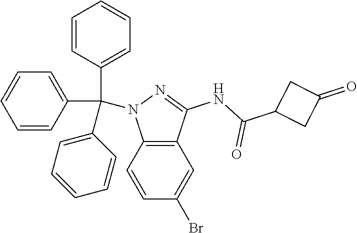 [SMILES: O=C1CC(C(=O)Nc2nn(C(c3ccccc3)(c3ccccc3)c3ccccc3)c3ccc(Br)cc23)C1]

Starting from 5-bromo-1-trityl-1H-indazol-3-ylamine and 3-oxocyclobutanecarboxylic acid. Flash column chromatography (Hexane/EtOAc 70:30). Obtained 0.55 g (91% of yield). 1H NMR (500 MHz, DMSO-d₆) δ ppm 3.26-3.31 (m, 2H), 3.44 (m, 1H), 6.34 (d, J=9.15 Hz, 1H), 7.19 (d, J=7.32 Hz, 7H), 7.27-7.30 (m, 3H), 7.32-7.36 (m, 6H), 8.05 (d, J=1.22 Hz, 1H), 10.90 (s, 1H). HRMS (ESI) calcd for C31H25BrN3O2Na [M+H+Na]⁺572.0944, found 572.0957.

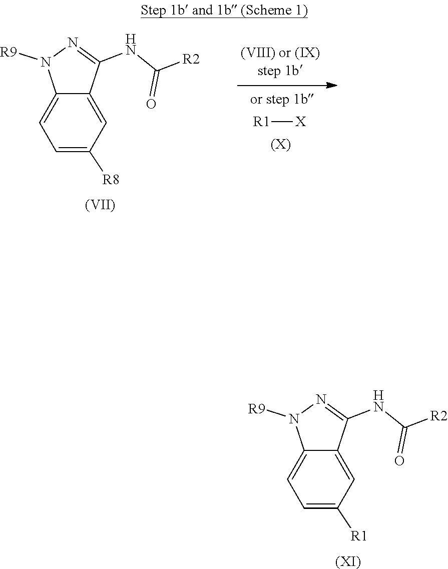

### tert-Butyl 3-{[5-(4-amino-2-chlorophenyl)-1-trityl-1H-indazol-3-yl]carbamoyl}piperidine-1-carboxylate [(XI), R1=4-amino-2-chlorophenyl, R2=tert-butyl piperidine-1-carboxylate, R9=1,1′0.1″-methanetriyltribenzene]

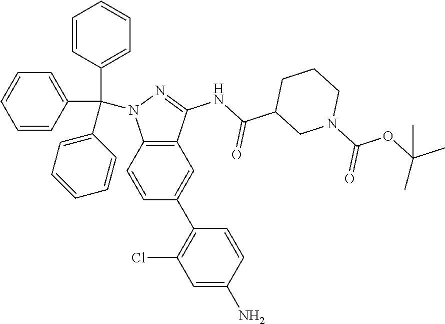 [SMILES: CC(C)(C)OC(=O)N1CCCC(C(=O)Nc2nn(C(c3ccccc3)(c3ccccc3)c3ccccc3)c3ccc(-c4ccc(N)cc4Cl)cc23)C1]

A solution of tert-butyl 3-[(5-bromo-1-trityl-1H-indazol-3-yl)carbamoyl]piperidine-1-carboxylate (0.060 g, 0.090 mmol) in mixture 1,4-dioxane/water 3:1 (4 mL) was evacuated with argon for 5 min. 3-Chloro-4-(4,4,5,5-tetramethyl-1,3,2-dioxaborolan-2-yl)aniline (0.068 g, 0.271 mmol), 1,1′-Bis(diphenylphosphino)ferrocene]dichloropalladium(II)×DCM (10% mol, 0.0074 g, 0.009 mmol) and Cs₂CO₃ (0.059 g, 0.180 mmol) were added. The mixture was heated, under argon, at 80° C., for 1 h, then was cooled, diluted with EtOAc (20 mL) and washed with aqueous NaHCO₃ (20 mL), water (20 mL) and brine (20 mL). The organic phase was separated, dried with anhydrous Na₂SO₄, filtered and evaporated to dryness. The crude was purified by flash column chromatography (Hexane/EtOAc 60:40) to obtain tert-butyl 3-{[5-(4-amino-2-chlorophenyl)-1-trityl-1H-indazol-3-yl]carbamoyl}piperidine-1-carboxylate (0.050 g, 78% of yield) as white solid. ¹H NMR (500 MHz, DMSO-d₆) δ ppm 1.37 (s, 9H), 1.51-1.76 (m, 2H), 1.92-1.99 (m, 2H), 2.57 (m, 1H), 2.73-2.79 (m 2H), 3.81-3.84 (m, 2H), 5.44 (s, 2H), 6.37 (d, J=9.00 Hz, 1H), 6.54 (dd, J=8.39, 2.29 Hz, 1H), 6.66 (d, J=2.29 Hz, 1H), 6.98 (d, J=8.39 Hz, 1H), 7.04 (d, J=9.91 Hz, 1H), 7.23 (d, J=7.63 Hz, 6H), 7.26-7.30 (m, 3H), 7.31-7.38 (m, 6H), 7.63 (s, 1H), 10.56 (s, 1H). HRMS (ESI) calcd for C43H43ClN5O3 [M+H]⁺ 712.3049, found 712.3026.

According to this same methodology, but employing suitable intermediates and reagents, the following compounds were prepared:

### N-[5-(2,4-Dichlorophenyl)-1-trityl-1H-indazol-3-yl]-1-methylpiperidine-3-carboxamide [(XI), R1=2,4-dichlorophenyl, R2=1-methylpiperidine, R9=1,1′, 1″-methanetriyltribenzene]

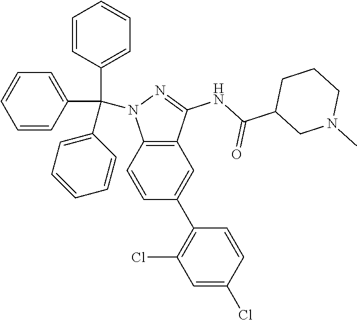 [SMILES: CN1CCCC(C(=O)Nc2nn(C(c3ccccc3)(c3ccccc3)c3ccccc3)c3ccc(-c4ccc(Cl)cc4Cl)cc23)C1]

Starting from N-(5-bromo-1-trityl-1H-indazol-3-yl)-1-methylpiperidine-3-carboxamide and (2,4-dichlorophenyl)boronic acid. Flash column chromatography (DCM/MeOH 90:10). Obtained 0.035 g (73% of yield). LCMS (ESI) m/z 645 (M+H)⁺.

### tert-Butyl 3-{[5-(2-chloro-4-hydroxyphenyl)-1-trityl-1H-indazol-3-yl]carbamoyl}piperidine-1-carboxylate [(XI), R1=2-chloro-4-hydroxyphenyl, R2=tert-butyl piperidine-1-carboxylate, R9=methanetriyltribenzene]

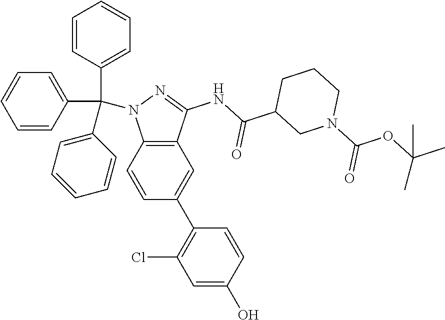 [SMILES: CC(C)(C)OC(=O)N1CCCC(C(=O)Nc2nn(C(c3ccccc3)(c3ccccc3)c3ccccc3)c3ccc(-c4ccc(O)cc4Cl)cc23)C1]

Starting from tert-butyl 3-[(5-bromo-1-trityl-1H-indazol-3-yl)carbamoyl]piperidine-1-carboxylate and (2-chloro-4-hydroxyphenyl)boronic acid. Flash column chromatography (Hexane/EtOAc 60:40). Obtained 0.058 g (74% of yield). ¹H NMR (500 MHz, DMSO-d₆) δ ppm 1.37 (s, 9H), 1.60-1.69 (m, 2H), 1.88-1.97 (m, 2H), 2.57 (m, 1H), 2.73-2.79 (m, 2H), 3.80-3.83 (m, 2H), 6.40 (d, J=8.85 Hz, 1H), 6.78 (dd, J=8.39, 2.44 Hz, 1H), 6.89 (d, J=2.44 Hz, 1H), 7.06 (d, J=9.30 Hz, 1H), 7.15 (d, J=8.24 Hz, 1H), 7.24 (d, J=7.63 Hz, 6H), 7.27-7.31 (m, 3H), 7.32-7.38 (m, 6H), 7.68 (s, 1H), 9.96 (s, 1H), 10.59 (s, 1H). HRMS (ESI) calcd for C43H42ClN4O4Na [M+H+Na]⁺735.2708, found 735.2701.

### tert-Butyl 4-{[5-(2-chloro-5-cyanophenyl)-1-trityl-1H-indazol-3-yl]carbamoyl}piperidine-1-carboxylate [(XI), R1=2-chloro-5-cyanophenyl, R2=tert-butyl piperidine-1-carboxylate, R9=1,1′, 1″-methanetriyltribenzene]

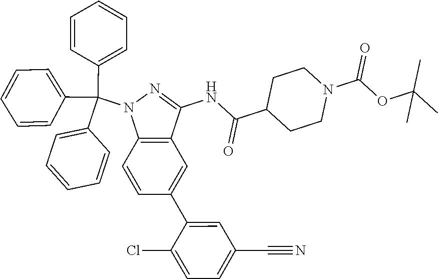 [SMILES: CC(C)(C)OC(=O)N1CCC(C(=O)Nc2nn(C(c3ccccc3)(c3ccccc3)c3ccccc3)c3ccc(-c4cc(C#N)ccc4Cl)cc23)CC1]

Starting from tert-butyl 4-[(5-bromo-1-trityl-1H-indazol-3-yl)carbamoyl]piperidine-1-carboxylate and (2-chloro-5-cyanophenyl)boronic acid. Flash column chromatography (Hexane/EtOAc 80:20). Obtained 0.055 g (78% of yield). LCMS (ESI) m/z 722 (M+H)⁺.

### tert-Butyl 3-{[5-(2,5-dichlorophenyl)-1-trityl-1H-indazol-3-yl]carbamoyl}piperidine-1-carboxylate [(XI), R1=2,5-dichlorophenyl, R2=tert-butyl piperidine-1-carboxylate, R9=1,1′, 1″-methanetriyltribenzene]

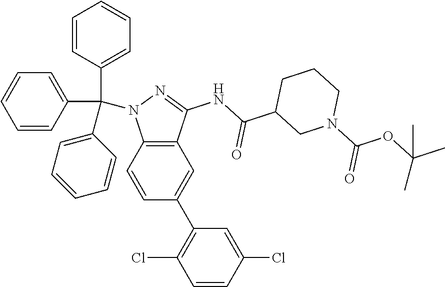 [SMILES: CC(C)(C)OC(=O)N1CCCC(C(=O)Nc2nn(C(c3ccccc3)(c3ccccc3)c3ccccc3)c3ccc(-c4cc(Cl)ccc4Cl)cc23)C1]

Starting from tert-butyl 3-[(5-bromo-1-trityl-1H-indazol-3-yl)carbamoyl]piperidine-1-carboxylate and (2,5-dichlorophenyl)boronic acid. Flash column chromatography (Hexane/EtOAc 80:20). Obtained 0.067 g (76% of yield). ¹H NMR (500 MHz, DMSO-d₆) δ ppm 1.30-1.36 (m, 10H), 1.56-1.76 (m, 2H), 1.94 (d, J=11.59 Hz, 1H), 2.58 (m, 1H), 2.77-2.81 (m, 2H), 3.79 (d, J=12.51 Hz, 1H), 3.93-3.96 (m, 2H), 6.45 (d, J=9.00 Hz, 1H), 7.14 (dd, J=8.92, 1.30 Hz, 1H), 7.24 (d, J=7.47 Hz, 6H), 7.27-7.32 (m, 3H), 7.32-7.39 (m, 6H), 7.41-7.45 (m, 1H), 7.46 (d, J=2.59 Hz, 1H), 7.57 (d, J=8.39 Hz, 1H), 7.82 (s, 1H), 10.64 (s, 1H). HRMS (ESI) calcd for C43H41Cl2N4O3Na [M+H+Na]⁺753.2369, found 753.2368.

### tert-Butyl (3R)-3-{[5-(4-amino-2-chlorophenyl)-1-trityl-1H-indazol-3-yl]carbamoyl}piperidine-1-carboxylate [(XI), R1=4-amino-2-chlorophenyl, R2=tert-butyl piperidine-1-carboxylate, R9=methanetriyltribenzene]

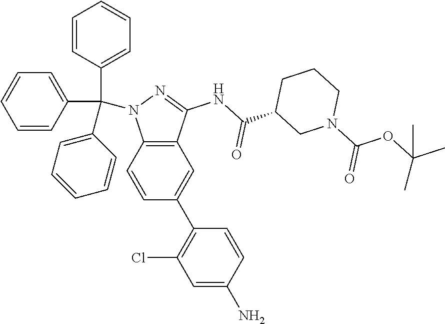 [SMILES: CC(C)(C)OC(=O)N1CCC[C@@H](C(=O)Nc2nn(C(c3ccccc3)(c3ccccc3)c3ccccc3)c3ccc(-c4ccc(N)cc4Cl)cc23)C1]

Starting from tert-butyl (3R)-3-[(5-bromo-1-trityl-1H-indazol-3-yl)carbamoyl]piperidine-1-carboxylate and 3-chloro-4-(4,4,5,5-tetramethyl-1,3,2-dioxaborolan-2-yl)aniline. Flash column chromatography (Hexane/EtOAc 60:40). Obtained 0.063 g (84% of yield). 1H NMR (500 MHz, DMSO-d₆) δ ppm 1.37 (s, 9H), 1.51-1.76 (m, 2H), 1.92-1.99 (m, 2H), 2.57 (m, 1H), 2.73-2.79 (m 2H), 3.81-3.84 (m, 2H), 5.44 (s, 2H), 6.37 (d, J=9.00 Hz, 1H), 6.54 (dd, J=8.39, 2.29 Hz, 1H), 6.66 (d, J=2.29 Hz, 1H), 6.98 (d, J=8.39 Hz, 1H), 7.04 (d, J=9.91 Hz, 1H), 7.23 (d, J=7.63 Hz, 6H), 7.26-7.30 (m, 3H), 7.31-7.38 (m, 6H), 7.63 (s, 1H), 10.56 (s, 1H). HRMS (ESI) calcd for C43H43ClN5O3 [M+H]⁺ 712.3049, found 712.3030.

### tert-Butyl (3R)-3-{[5-(2-chloro-4-hydroxyphenyl)-1-trityl-1H-indazol-3-yl]carbamoyl}piperidine-1-carboxylate [(XI), R1=2-chloro-4-hydroxyphenyl, R2=tert-butyl piperidine-1-carboxylate, R9=methanetriyltribenzene]

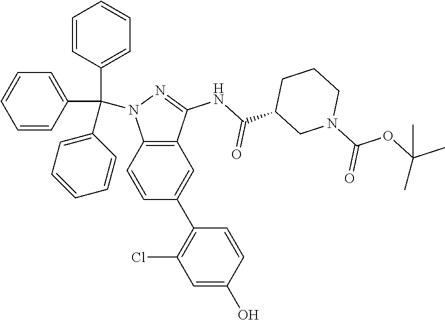 [SMILES: CC(C)(C)OC(=O)N1CCC[C@@H](C(=O)Nc2nn(C(c3ccccc3)(c3ccccc3)c3ccccc3)c3ccc(-c4ccc(O)cc4Cl)cc23)C1]

Starting from tert-butyl (3R)-3-[(5-bromo-1-trityl-1H-indazol-3-yl)carbamoyl]piperidine-1-carboxylate and (2-chloro-4-hydroxyphenyl)boronic acid. Flash column chromatography (Hexane/EtOAc 60:40). Obtained 0.060 g (80% of yield). 1H NMR (500 MHz, DMSO-d₆) δ ppm 1.37 (s, 9H), 1.60-1.69 (m, 2H), 1.88-1.97 (m, 2H), 2.57 (m, 1H), 2.73-2.79 (m, 2H), 3.80-3.83 (m, 2H), 6.40 (d, J=8.85 Hz, 1H), 6.78 (dd, J=8.39, 2.44 Hz, 1H), 6.89 (d, J=2.44 Hz, 1H), 7.06 (d, J=9.30 Hz, 1H), 7.15 (d, J=8.24 Hz, 1H), 7.24 (d, J=7.63 Hz, 6H), 7.27-7.31 (m, 3H), 7.32-7.38 (m, 6H), 7.68 (s, 1H), 9.96 (s, 1H), 10.59 (s, 1H). HRMS (ESI) calcd for C43H42ClN4O [M+H]⁺ 713.2889, found 713.2870.

### tert-Butyl (3S)-3-{[5-(4-amino-2-chlorophenyl)-1-trityl-1H-indazol-3-yl]carbamoyl}piperidine-1-carboxylate [(XI), R1=4-amino-2-chlorophenyl, R2=tert-butyl piperidine-1-carboxylate, R9=methanetriyltribenzene]

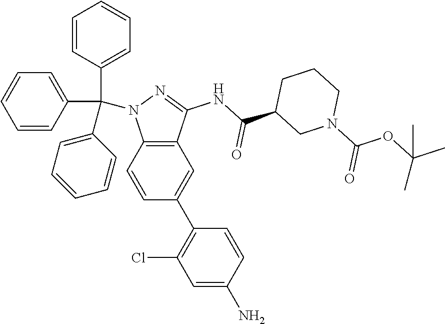 [SMILES: CC(C)(C)OC(=O)N1CCC[C@H](C(=O)Nc2nn(C(c3ccccc3)(c3ccccc3)c3ccccc3)c3ccc(-c4ccc(N)cc4Cl)cc23)C1]

Starting from tert-butyl (3S)-3-[(5-bromo-1-trityl-1H-indazol-3-yl)carbamoyl]piperidine-1-carboxylate and 3-chloro-4-(4,4,5,5-tetramethyl-1,3,2-dioxaborolan-2-yl)aniline. Flash column chromatography (Hexane/EtOAc 60:40). Obtained 0.059 g (79% of yield). ¹H NMR (500 MHz, DMSO-d₆) δ ppm 1.37 (s, 9H), 1.51-1.76 (m, 2H), 1.92-1.99 (m, 2H), 2.57 (m, 1H), 2.73-2.79 (m, 2H), 3.81-3.84 (m, 2H), 5.44 (s, 2H), 6.37 (d, J=9.00 Hz, 1H), 6.54 (dd, J=8.39, 2.29 Hz, 1H), 6.66 (d, J=2.29 Hz, 1H), 6.98 (d, J=8.39 Hz, 1H), 7.04 (d, J=9.91 Hz, 1H), 7.23 (d, J=7.63 Hz, 6H), 7.26-7.30 (m, 3H), 7.31-7.38 (m, 6H), 7.63 (s, 1H), 10.56 (s, 1H). HRMS (ESI) calcd for C43H43ClN5O3 [M+H]⁺ 712.3049, found 712.3031.

### tert-Butyl (3R)-3-{[5-(2-chloro-4-methylphenyl)-1-trityl-1H-indazol-3-yl]carbamoyl}piperidine-1-carboxylate [(XI), R1=2-chloro-4-methylphenyl, R2=tert-butyl piperidine-1-carboxylate, R9=methanetriyltribenzene]

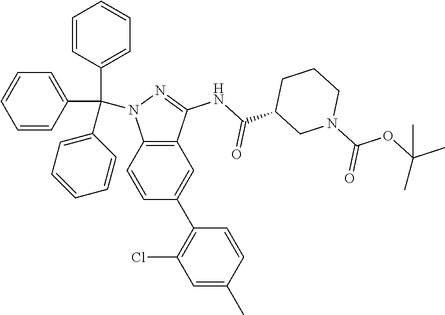 [SMILES: Cc1ccc(-c2ccc3c(c2)c(NC(=O)[C@@H]2CCCN(C(=O)OC(C)(C)C)C2)nn3C(c2ccccc2)(c2ccccc2)c2ccccc2)c(Cl)c1]

Starting from tert-butyl (3R)-3-[(5-bromo-1-trityl-1H-indazol-3-yl)carbamoyl]piperidine-1-carboxylate and (2-chloro-5-methylphenyl)boronic acid. Flash column chromatography (Hexane/EtOAc 80:20). Obtained 0.060 g (75% of yield). LCMS (ESI) m/z 711 (M+H)⁺.

### tert-Butyl 3-{[5-(5-carbamoyl-2-chlorophenyl)-1-trityl-1H-indazol-3-yl]carbamoyl}piperidine-1-carboxylate [(XI), R1=5-carbamoyl-2-chlorophenyl, R2=tert-butyl piperidine-1-carboxylate, R9=methanetriyltribenzene]

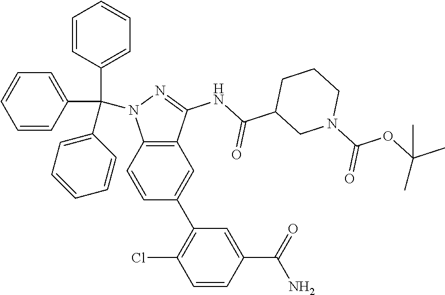 [SMILES: CC(C)(C)OC(=O)N1CCCC(C(=O)Nc2nn(C(c3ccccc3)(c3ccccc3)c3ccccc3)c3ccc(-c4cc(C(N)=O)ccc4Cl)cc23)C1]

Starting from tert-butyl 3-[(5-bromo-1-trityl-1H-indazol-3-yl)carbamoyl]piperidine-1-carboxylate and (5-carbamoyl-2-chlorophenyl)boronic acid. Flash column chromatography (Hexane/EtOAc 70:30). Obtained 0.077 g (83% of yield). LCMS (ESI) m/z 740 (M+H)⁺.

### tert-Butyl (3S)-3-{[5-(2-chloro-4-hydroxyphenyl)-1-trityl-1H-indazol-3-yl]carbamoyl}piperidine-1-carboxylate [(XI), R1=2-chloro-4-hydroxyphenyl, R2=tert-butyl piperidine-1-carboxylate, R9=methanetriyltribenzene]

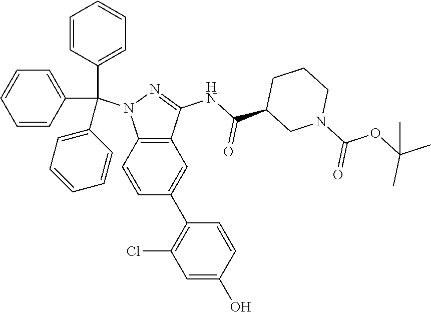 [SMILES: CC(C)(C)OC(=O)N1CCC[C@H](C(=O)Nc2nn(C(c3ccccc3)(c3ccccc3)c3ccccc3)c3ccc(-c4ccc(O)cc4Cl)cc23)C1]

Starting from tert-butyl (3S)-3-[(5-bromo-1-trityl-1H-indazol-3-yl)carbamoyl]piperidine-1-carboxylate and (2-chloro-4-hydroxyphenyl)boronic acid. Flash column chromatography (Hexane/EtOAc 60:40). Obtained 0.064 g (85% of yield). ¹H NMR (500 MHz, DMSO-d₆) δ ppm 1.37 (s, 9H), 1.60-1.69 (m, 2H), 1.88-1.97 (m, 2H), 2.57 (m, 1H), 2.73-2.79 (m, 2H), 3.80-3.83 (m, 2H), 6.40 (d, J=8.85 Hz, 1H), 6.78 (dd, J=8.39, 2.44 Hz, 1H), 6.89 (d, J=2.44 Hz, 1H), 7.06 (d, J=9.30 Hz, 1H), 7.15 (d, J=8.24 Hz, 1H), 7.24 (d, J=7.63 Hz, 6H), 7.27-7.31 (m, 3H), 7.32-7.38 (m, 6H), 7.68 (s, 1H), 9.96 (s, 1H), 10.59 (s, 1H). HRMS (ESI) calcd for C43H42ClN4ONa [M+H+Na]⁺735.2708, found 735.2687.

### tert-Butyl 3-{[5-(3-amino-2-fluorophenyl)-1-trityl-1H-indazol-3-yl]carbamoyl}piperidine-1-carboxylate [(XI), R1=3-amino-2-fluorophenyl, R2=tert-butyl piperidine-1-carboxylate, R9=1,1′, 1″-methanetriyltribenzene]

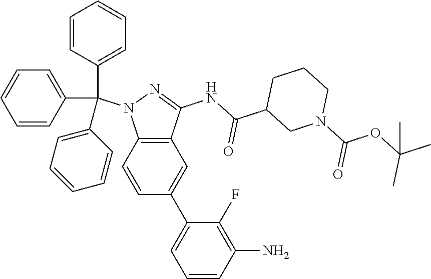 [SMILES: CC(C)(C)OC(=O)N1CCCC(C(=O)Nc2nn(C(c3ccccc3)(c3ccccc3)c3ccccc3)c3ccc(-c4cccc(N)c4F)cc23)C1]

Starting from tert-butyl 3-[(5-bromo-1-trityl-1H-indazol-3-yl)carbamoyl]piperidine-1-carboxylate and (3-ammino-2-fluorophenyl)boronic acid. Flash column chromatography (Hexane/EtOAc 80:20). Obtained 0.048 g (72% of yield). LCMS (ESI) m/z 696 (M+H)⁺.

### tert-Butyl (3S)-3-{[5-(2-chloro-4-methylphenyl)-1-trityl-1H-indazol-3-yl]carbamoyl}piperidine-1-carboxylate [(XI), R1=2-chloro-4-methylphenyl, R2=tert-butyl piperidine-1-carboxylate, R9=methanetriyltribenzene]

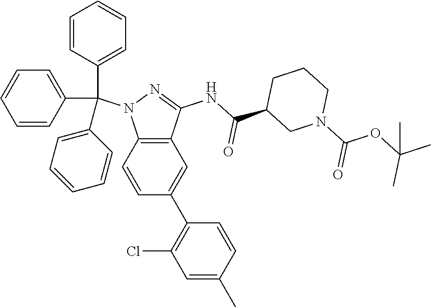 [SMILES: Cc1ccc(-c2ccc3c(c2)c(NC(=O)[C@H]2CCCN(C(=O)OC(C)(C)C)C2)nn3C(c2ccccc2)(c2ccccc2)c2ccccc2)c(Cl)c1]

Starting from tert-butyl (3S)-3-[(5-bromo-1-trityl-1H-indazol-3-yl)carbamoyl]piperidine-1-carboxylate and (2-chloro-5-methylphenyl)boronic acid. Flash column chromatography (Hexane/EtOAc 80:20). Obtained 0.052 g (76% of yield). LCMS (ESI) m/z 711 (M+H)⁺.

### tert-Butyl (3R)-3-{[5-(2,5-dichlorophenyl)-1-trityl-1H-indazol-3-yl]carbamoyl}piperidine-1-carboxylate [(XI), R1=2,5-dichlorophenyl, R2=tert-butyl piperidine-1-carboxylate, R9=1,1′,1″-methanetriyltribenzene]

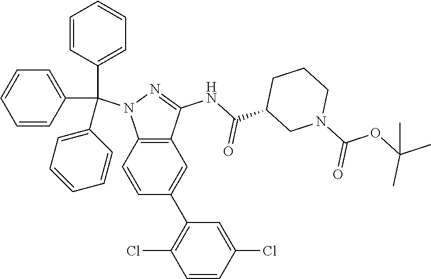 [SMILES: CC(C)(C)OC(=O)N1CCC[C@@H](C(=O)Nc2nn(C(c3ccccc3)(c3ccccc3)c3ccccc3)c3ccc(-c4cc(Cl)ccc4Cl)cc23)C1]

Starting from tert-butyl (3R)-3-[(5-bromo-1-trityl-1H-indazol-3-yl)carbamoyl]piperidine-1-carboxylate and (2,5-dichlorophenyl)boronic acid. Flash column chromatography (Hexane/EtOAc 80:20). Obtained 0.064 g (77% of yield). ¹H NMR (500 MHz, DMSO-d₆) δ ppm 1.30-1.36 (m, 10H), 1.56-1.76 (m, 2H), 1.94 (d, J=11.59 Hz, 1H), 2.58 (m, 1H), 2.77-2.81 (m, 2H), 3.79 (d, J=12.51 Hz, 1H), 3.93-3.96 (m, 2H), 6.45 (d, J=9.00 Hz, 1H), 7.14 (dd, J=8.92, 1.30 Hz, 1H), 7.24 (d, J=7.47 Hz, 6H), 7.27-7.32 (m, 3H), 7.32-7.39 (m, 6H), 7.41-7.45 (m, 1H), 7.46 (d, J=2.59 Hz, 1H), 7.57 (d, J=8.39 Hz, 1H), 7.82 (s, 1H), 10.64 (s, 1H). HRMS (ESI) calcd for C43H41Cl2N4O3Na [M+H+Na]⁺753.2369, found 753.2366.

### tert-Butyl (3R)-3-{[5-(2,4-dichlorophenyl)-1-trityl-1H-indazol-3-yl]carbamoyl}piperidine-1-carboxylate [(XI), R1=2,4-dichlorophenyl, R2=tert-butyl piperidine-1-carboxylate, R9=1,1′, 1″-methanetriyltribenzene]

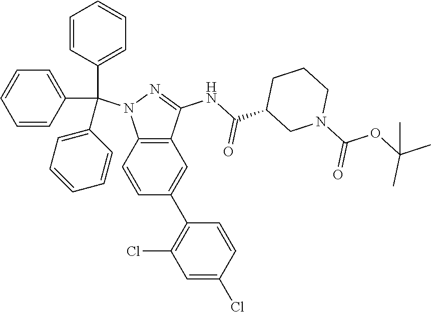 [SMILES: CC(C)(C)OC(=O)N1CCC[C@@H](C(=O)Nc2nn(C(c3ccccc3)(c3ccccc3)c3ccccc3)c3ccc(-c4ccc(Cl)cc4Cl)cc23)C1]

Starting from tert-butyl (3R)-3-[(5-bromo-1-trityl-1H-indazol-3-yl)carbamoyl]piperidine-1-carboxylate and (2,4-dichlorophenyl)boronic acid. Flash column chromatography (Hexane/EtOAc 80:20). Obtained 0.056 g (64% of yield). ¹H NMR (500 MHz, DMSO-d₆) δ ppm 1.26-1.36 (m, 10H), 1.57-1.79 (m, 2H), 1.94 (d, J=13.57 Hz, 1H), 2.58 (m, 1H), 2.73-2.82 (m, 1H), 3.80 (d, J=13.12 Hz, 1H), 3.85-4.09 (m, 2H), 6.44 (d, J=9.00 Hz, 1H), 7.12 (d, J=8.85 Hz, 1H), 7.24 (d, J=7.63 Hz, 6H), 7.27-7.31 (m, 3H), 7.32-7.37 (m, 6H), 7.39 (d, J=8.39 Hz, 1H), 7.47 (dd, J=8.39, 2.14 Hz, 1H), 7.70 (d, J=2.13 Hz, 1H), 7.79 (s, 1H), 10.64 (s, 1H). HRMS (ESI) calcd for C43H41Cl2N4O3Na [M+H+Na]⁺753.2369, found 753.2379.

### tert-Butyl 3-({5-[5-chloro-2-(trifluoromethyl)phenyl]-1-trityl-1H-indazol-3-yl)carbamoyl)piperidine-1-carboxylate [(XI), R1=5-chloro-2-(trifluoromethyl)phenyl, R2=tert-butyl piperidine-1-carboxylate, R9=1,1′, 1″-methanetriyltribenzene]

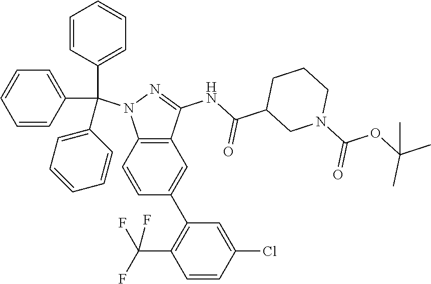 [SMILES: CC(C)(C)OC(=O)N1CCCC(C(=O)Nc2nn(C(c3ccccc3)(c3ccccc3)c3ccccc3)c3ccc(-c4cc(Cl)ccc4C(F)(F)F)cc23)C1]

Starting from tert-butyl 3-[(5-bromo-1-trityl-1H-indazol-3-yl)carbamoyl]piperidine-1-carboxylate and [5-chloro-2-(trifluoromethyl)phenyl]boronic acid. Flash column chromatography (Hexane/EtOAc 80:20). Obtained 0.055 g (68% of yield). ¹H NMR (500 MHz, DMSO-d₆) δ ppm 1.35-1.39 (m, 10H), 1.57-1.71 (m, 2H), 1.92 (m, 1H), 2.57 (m, 1H), 2.76-2.84 (m, 1H), 3.76-3.80 (m, 2H), 3.90-3.97 (m, 1H), 6.32 (d, J=9.15 Hz, 1H), 6.41 (d, J=9.00 Hz, 1H), 7.03 (d, J=8.69 Hz, 1H), 7.17-7.18 (m, 4H), 7.23-7.24 (m, 3H), 7.27-7.36 (m, 6H), 7.45 (s, 1H), 7.67 (dd, J=8.69, 1.68 Hz, 1H), 7.72 (s, 1H), 7.82 (d, J=8.54 Hz, 1H), 7.93 (s, 1H), 10.63 (s, 1H). HRMS (ESI) calcd for C44H41ClF3N4O3Na [M+H+Na]⁺787.2633, found 787.2622.

### tert-Butyl 3-{[5-(5-acetyl-2-fluorophenyl)-1-trityl-1H-indazol-3-yl]carbamoyl}piperidine-1-carboxylate [(XI), R1=5-acetyl-2-fluorophenyl, R2=tert-butyl piperidine-1-carboxylate, R9=1,1′, 1″-methanetriyltribenzene]

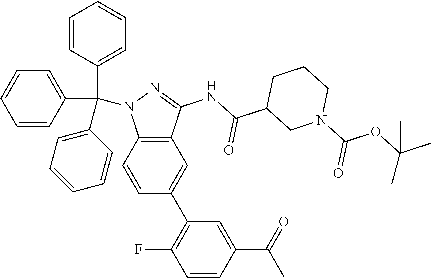 [SMILES: CC(=O)c1ccc(F)c(-c2ccc3c(c2)c(NC(=O)C2CCCN(C(=O)OC(C)(C)C)C2)nn3C(c2ccccc2)(c2ccccc2)c2ccccc2)c1]

Starting from tert-butyl 3-[(5-bromo-1-trityl-1H-indazol-3-yl)carbamoyl]piperidine-1-carboxylate and (5-acetyl-2-fluorophenyl)boronic acid. Flash column chromatography (Hexane/EtOAc 80:20). Obtained 0.089 g (96% of yield). ¹H NMR (500 MHz, DMSO-d₆) δ ppm 1.33-1.36 (m, 10H), 1.56-1.76 (m, 2H), 1.96 (d, J=12.81 Hz, 1H), 2.60 (m, 1H), 2.78-2.81 (m, 1H), 2.89 (s, 3H), 3.79 (d, J=12.96 Hz, 1H), 3.92-4.03 (m, 2H), 6.48 (d, J=9.00 Hz, 1H), 7.23-7.37 (m, 16H), 7.41-7.48 (m, 1H), 7.93-7.99 (m, 3H), 10.65 (s, 1H). HRMS (ESI) calcd for C45H44FN4O4Na [M+H+Na]⁺745.3160, found 745.3149.

### tert-Butyl 3-{[5-(2-chloro-5-methoxyphenyl)-1-trityl-1H-indazol-3-yl]carbamoyl}piperidine-1-carboxylate [(XI), R1=2-chloro-5-methoxyphenyl, R2=tert-butyl piperidine-1-carboxylate, R9=methanetriyltribenzene]

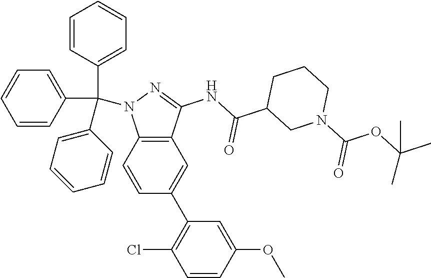 [SMILES: COc1ccc(Cl)c(-c2ccc3c(c2)c(NC(=O)C2CCCN(C(=O)OC(C)(C)C)C2)nn3C(c2ccccc2)(c2ccccc2)c2ccccc2)c1]

Starting from tert-butyl 3-[(5-bromo-1-trityl-1H-indazol-3-yl)carbamoyl]piperidine-1-carboxylate and (2-chloro-5-methoxyphenyl)boronic acid. Flash column chromatography (Hexane/EtOAc 80:20). Obtained 0.072 g (94% of yield). ¹H NMR (500 MHz, DMSO-d₆) δ ppm 1.30-1.36 (m, 10H), 1.59-1.69 (m, 2H), 1.93 (d, J=14.03 Hz, 1H), 2.58 (m, 1H), 2.74-2.81 (m, 1H), 3.76 (s, 3H), 3.79 (d, J=12.81 Hz, 1H), 3.90-3.98 (m, 2H), 6.44 (d, J=9.00 Hz, 1H), 6.88 (d, J=2.90 Hz, 1H), 6.95 (dd, J=8.85, 3.05 Hz, 1H), 7.13 (d, J=8.69 Hz, 1H), 7.24 (d, J=7.63 Hz, 6H), 7.28-7.30 (m, 3H), 7.33-7.36 (m, 6H), 7.42 (d, J=8.85 Hz, 1H), 7.78 (s, 1H), 10.62 (s, 1H). HRMS (ESI) calcd for C44H44ClN4O4 [M+H]⁺ 727.3046, found 727.3045.

### tert-Butyl (3R)-3-{[5-(2-chloro-5-methoxyphenyl)-1-trityl-1H-indazol-3-yl]carbamoyl}piperidine-1-carboxylate [(XI), R1=2-chloro-5-methoxyphenyl, R2=tert-butyl piperidine-1-carboxylate, R9=methanetriyltribenzene]

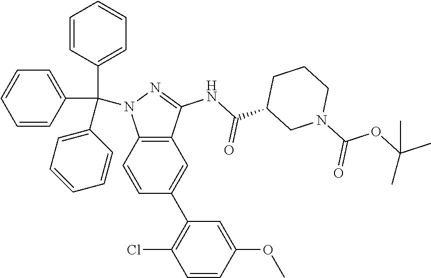 [SMILES: COc1ccc(Cl)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and (2-chloro-5-methoxyphenyl)boronic acid. Flash column chromatography (Hexane/EtOAc 80:20). Obtained 0.063 g (95% of yield). ¹H NMR (500 MHz, DMSO-d₆) δ ppm 1.30-1.36 (m, 10H), 1.59-1.69 (m, 2H), 1.93 (d, J=14.03 Hz, 1H), 2.58 (m, 1H), 2.74-2.81 (m, 1H), 3.76 (s, 3H), 3.79 (d, J=12.81 Hz, 1H), 3.90-3.98 (m, 2H), 6.44 (d, J=9.00 Hz, 1H), 6.88 (d, J=2.90 Hz, 1H), 6.95 (dd, J=8.85, 3.05 Hz, 1H), 7.13 (d, J=8.69 Hz, 1H), 7.24 (d, J=7.63 Hz, 6H), 7.28-7.30 (m, 3H), 7.33-7.36 (m, 6H), 7.42 (d, J=8.85 Hz, 1H), 7.78 (s, 1H), 10.62 (s, 1H). HRMS (ESI) calcd for C44H44ClN4O4 [M+H]⁺ 727.3046, found 727.3042.

### tert-Butyl (3R)-3-{[5-(5-acetyl-2-fluorophenyl)-1-trityl-1H-indazol-3-yl]carbamoyl}piperidine-1-carboxylate [(XI), R1=5-acetyl-2-fluorophenyl, R2=tert-butyl piperidine-1-carboxylate, R9=methanetriyltribenzene]

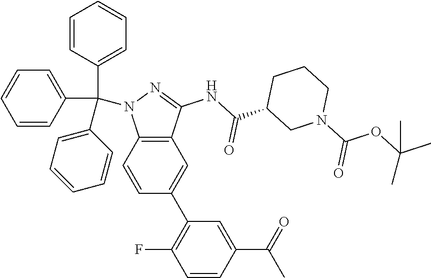 [SMILES: CC(=O)c1ccc(F)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and (5-acetyl-2-fluorophenyl)boronic acid. Flash column chromatography (Hexane/EtOAc 80:20). Obtained 0.110 g (91% of yield). 1H NMR (500 MHz, DMSO-d₆) δ ppm 1.33-1.36 (m, 10H), 1.56-1.76 (m, 2H), 1.96 (d, J=12.81 Hz, 1H), 2.60 (m, 1H), 2.78-2.81 (m, 1H), 2.89 (s, 3H), 3.79 (d, J=12.96 Hz, 1H), 3.92-4.03 (m, 2H), 6.48 (d, J=9.00 Hz, 1H), 7.23-7.37 (m, 16H), 7.41-7.48 (m, 1H), 7.93-7.99 (m, 3H), 10.65 (s, 1H). HRMS (ESI) calcd for C45H44FN4O4Na [M+H+Na]⁺745.3160, found 745.3155.

### tert-Butyl (3R)-3-{[5-(2-fluoro-5-methoxyphenyl)-1-trityl-1H-indazol-3-yl]carbamoyl}piperidine-1-carboxylate [(XI), R1=2-fluoro-5-methoxyphenyl, R2=tert-butyl piperidine-1-carboxylate, R9=methanetriyltribenzene]

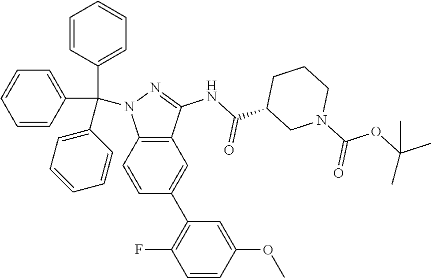 [SMILES: COc1ccc(F)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and (2-fluoro-5-methoxyphenyl)boronic acid. Flash column chromatography (Hexane/EtOAc 80:20). Obtained 0.099 g (93% of yield). ¹H NMR (500 MHz, DMSO-d₆) δ ppm 1.27-1.40 (m, 10H), 1.61-1.71 (m, 2H), 1.95 (d, J=12.66 Hz, 1H), 2.59 (m, 1H), 2.74-2.80 (m, 1H), 3.76 (s, 3H), 3.80 (d, J=12.81 Hz, 1H), 3.95-4.00 (m, 2H), 6.45 (d, J=9.15 Hz, 1H), 6.86-6.97 (m, 2H), 7.15-7.26 (m, 8H), 7.27-7.32 (m, 3H), 7.32-7.40 (m, 6H), 7.87 (s, 1H), 10.63 (s, 1H). HRMS (ESI) calcd for C44H44FN4O4Na [M+H+Na]⁺745.3160, found 745.3163.

### tert-Butyl (3R)-3-{[5-(5-chloro-2-fluorophenyl)-1-trityl-1H-indazol-3-yl]carbamoyl}piperidine-1-carboxylate [(XI), R1=5-chloro-2-fluorophenyl, R2=tert-butyl piperidine-1-carboxylate, R9=methanetriyltribenzene]

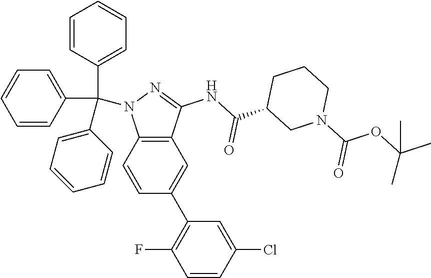 [SMILES: CC(C)(C)OC(=O)N1CCC[C@@H](C(=O)Nc2nn(C(c3ccccc3)(c3ccccc3)c3ccccc3)c3ccc(-c4cc(Cl)ccc4F)cc23)C1]

Starting from tert-butyl (3R)-3-[(5-bromo-1-trityl-1H-indazol-3-yl)carbamoyl]piperidine-1-carboxylate and (5-chloro-2-fluorophenyl)boronic acid. Flash column chromatography (Hexane/EtOAc 80:20). Obtained 0.055 g (87% of yield). ¹H NMR (500 MHz, DMSO-d₆) δ ppm 1.29-1.40 (m, 10H), 1.64-1.71 (m, 2H), 1.92-1.97 (m, 1H), 2.59 (m, 1H), 2.75-2.81 (m, 1H), 3.80 (d, J=11.59 Hz, 1H), 3.96-3.99 (m, 2H), 6.46 (d, J=9.00 Hz, 1H), 7.19-7.27 (m, 7H), 7.27-7.31 (m, 3H), 7.33-7.37 (m, 7H), 7.42-7.47 (m, 1H), 7.49-7.54 (m, 1H), 7.92 (s, 1H), 10.65 (s, 1H). HRMS (ESI) calcd for C43H41ClFN4O3Na [M+H+Na]⁺737.2665, found 737.2661.

### tert-Butyl (3R)-3-{[5-(5-ethoxy-2-fluorophenyl)-1-trityl-1H-indazol-3-yl]carbamoyl}piperidine-1-carboxylate [(XI), R1=5-ethoxy-2-fluorophenyl, R2=tert-butyl piperidine-1-carboxylate, R9=methanetriyltribenzene]

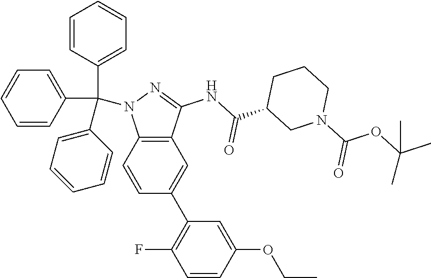 [SMILES: CCOc1ccc(F)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and (5-ethoxy-2-fluorophenyl)boronic acid. Flash column chromatography (Hexane/EtOAc 80:20). Obtained 0.066 g (83% of yield). ¹H NMR (500 MHz, DMSO-d₆) δ ppm 1.28-1.33 (m, 4H), 1.37 (s, 9H), 1.61-1.70 (m, 2H), 1.94-1.98 (m, 1H), 2.59 (m, 1H), 2.76-2.81 (m, 1H), 3.81 (d, J=11.44 Hz, 1H), 3.95-4.00 (m, 2H), 4.02 (q, J=6.91 Hz, 2H), 6.44 (d, J=9.00 Hz, 1H), 6.84-6.94 (m, 2H), 7.18 (t, J=9.61 Hz, 1H), 7.22 (d, J=7.78 Hz, 6H), 7.28-7.30 (m, 4H), 7.33-7.36 (m, 6H), 7.87 (s, 1H), 10.62 (s, 1H). HRMS (ESI) calcd for C45H46FN4O4Na [M+H+Na]⁺747.3317, found 747.3312.

### tert-Butyl (3R)-3-({5-[2-fluoro-5-(trifluoromethoxy)phenyl]-1-trityl-1H-indazol-3-yl}carbamoyl)piperidine-1-carboxylate [(XI), R1=2-fluoro-5-(trifluoromethoxy)phenyl, R2=tert-butyl piperidine-1-carboxylate, R9=1,1′,1″-methanetriyltribenzene]

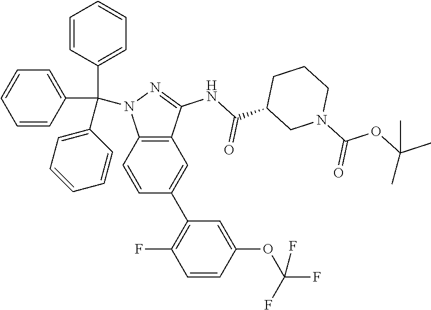 [SMILES: CC(C)(C)OC(=O)N1CCC[C@@H](C(=O)Nc2nn(C(c3ccccc3)(c3ccccc3)c3ccccc3)c3ccc(-c4cc(OC(F)(F)F)ccc4F)cc23)C1]

Starting from tert-butyl (3R)-3-[(5-bromo-1-trityl-1H-indazol-3-yl)carbamoyl]piperidine-1-carboxylate and [2-fluoro-5-(trifluoromethoxy)phenyl]boronic acid. Flash column chromatography (Hexane/EtOAc 80:20). Obtained 0.110 g (89% of yield). ¹H NMR (500 MHz, DMSO-d₆) δ ppm 1.31-1.36 (m, 10H), 1.59-1.74 (m, 2H), 1.92-1.97 (m, 1H), 2.59 (m, 1H), 2.76-2.84 (m, 1H), 3.79 (d, J=13.42 Hz, 1H), 3.89-3.97 (m, 2H), 6.47 (d, J=9.00 Hz, 1H), 7.17 (d, J=7.47 Hz, 1H), 7.22 (d, J=7.78 Hz, 6H), 7.28-7.31 (m, 3H), 7.33-7.36 (m, 6H), 7.40-7.51 (m, 3H), 7.93 (s, 1H), 10.66 (s, 1H). HRMS (ESI) calcd for C44H41F4N4O4Na [M+H+Na]⁺787.2878, found 787.2872.

### tert-Butyl (3R)-3-{[5-(5-cyano-2-methylphenyl)-1-trityl-1H-indazol-3-yl]carbamoyl}piperidine-1-carboxylate [(XI), R1=5-cyano-2-methylphenyl, R2=tert-butyl piperidine-1-carboxylate, R9=methanetriyltribenzene]

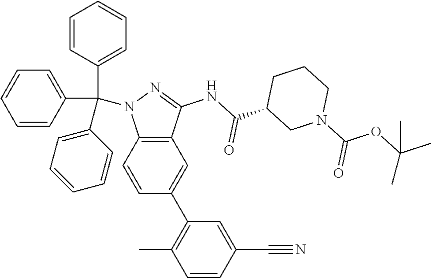 [SMILES: Cc1ccc(C#N)cc1-c1ccc2c(c1)c(NC(=O)[C@@H]1CCCN(C(=O)OC(C)(C)C)C1)nn2C(c1ccccc1)(c1ccccc1)c1ccccc1]

Starting from tert-butyl (3R)-3-[(5-bromo-1-trityl-1H-indazol-3-yl)carbamoyl]piperidine-1-carboxylate and (5-cyano-2-methylphenyl)boronic acid. Flash column chromatography (Hexane/EtOAc 70:30). Obtained 0.060 g (87% of yield). ¹H NMR (500 MHz, DMSO-d₆) δ ppm 1.30-1.35 (m, 10H), 1.55-1.78 (m, 2H), 1.92-1.95 (m, 1H), 2.24 (s, 3H), 2.58 (m, 1H), 2.76-2.81 (m, 1H), 3.76 (d, J=12.66 Hz, 1H), 3.91-3.97 (m, 2H), 6.44 (d, J=9.00 Hz, 1H), 7.09 (d, J=9.15 Hz, 1H), 7.24 (d, J=7.63 Hz, 6H), 7.28-7.31 (m, 3H), 7.33-7.36 (m, 6H), 7.49 (m, 1H), 7.59 (s, 1H), 7.70-7.71 (m, 2H), 10.62 (s, 1H). HRMS (ESI) calcd for C45H44N5O3Na [M+H+Na]⁺724.3258, found 724.3259.

### tert-Butyl (3R)-3-{[5-(5-cyano-2-fluorophenyl)-1-trityl-1H-indazol-3-yl]carbamoyl}piperidine-1-carboxylate [(XI), R1=5-cyano-2-fluorophenyl, R2=tert-butyl piperidine-1-carboxylate, R9=methanetriyltribenzene]

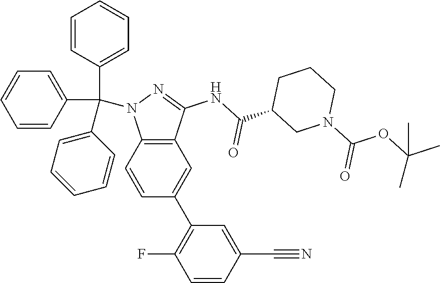 [SMILES: CC(C)(C)OC(=O)N1CCC[C@@H](C(=O)Nc2nn(C(c3ccccc3)(c3ccccc3)c3ccccc3)c3ccc(-c4cc(C#N)ccc4F)cc23)C1]

Starting from tert-butyl (3R)-3-[(5-bromo-1-trityl-1H-indazol-3-yl)carbamoyl]piperidine-1-carboxylate and (5-cyano-2-fluorophenyl)boronic acid. Flash column chromatography (Hexane/EtOAc 70:30). Obtained 0.037 g (54% of yield). LCMS (ESI) m/z 706 (M+H)⁺.

### tert-Butyl (3R)-3-{[5-(2-chloro-5-nitrophenyl)-1-trityl-1H-indazol-3-yl]carbamoyl}piperidine-1-carboxylate [(XI), R1=2-chloro-5-nitrophenyl, R2=tert-butyl piperidine-1-carboxylate, R9=

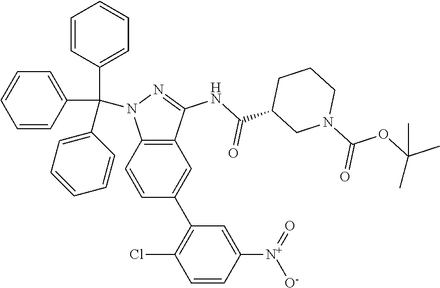 [SMILES: CC(C)(C)OC(=O)N1CCC[C@@H](C(=O)Nc2nn(C(c3ccccc3)(c3ccccc3)c3ccccc3)c3ccc(-c4cc([N+](=O)[O-])ccc4Cl)cc23)C1]

Starting from tert-butyl (3R)-3-[(5-bromo-1-trityl-1H-indazol-3-yl)carbamoyl]piperidine-1-carboxylate and (2-chloro-5-nitrophenyl)boronic acid. Flash column chromatography (Hexane/EtOAc 70:30). Obtained 0.060 g (83% of yield). ¹H NMR (500 MHz, DMSO-d₆) δ ppm 1.23-1.43 (m, 10H), 1.57-1.73 (m, 2H), 1.89-1.98 (m, 1H), 2.59 (m, 1H), 2.76-2.84 (m, 1H), 3.77 (d, J=13.42 Hz, 1H), 3.93-3.98 (m, 2H), 6.49 (d, J=9.00 Hz, 1H), 7.20 (d, J=9.00 Hz, 1H), 7.24 (d, J=7.63 Hz, 6H), 7.28-7.31 (m, 3H), 7.34-7.37 (m, 6H), 7.86 (d, J=8.85 Hz, 1H), 7.91 (s, 1H), 8.11 (d, J=2.44 Hz, 1H), 8.21 (dd, J=8.85, 2.75 Hz, 1H), 10.67 (s, 1H). HRMS (ESI) calcd for C43H41ClN5O5Na [M+H+Na]⁺764.2610, found 764.2593.

### N-[5-(2,6-Dichlorophenyl)-1-trityl-1H-indazol-3-yl]-6-oxopiperidine-3-carboxamide [(XI), R1=2,6-dichlorophenyl, R2=piperidin-2-one, R9=1,1′,1″-methanetriyltribenzene]

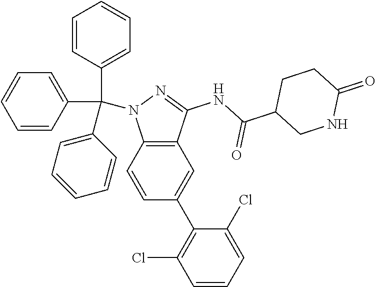 [SMILES: O=C1CCC(C(=O)Nc2nn(C(c3ccccc3)(c3ccccc3)c3ccccc3)c3ccc(-c4c(Cl)cccc4Cl)cc23)CN1]

Starting from N-(5-bromo-1-trityl-1H-indazol-3-yl)-6-oxopiperidine-3-carboxamide and (2,6-dichlorophenyl)boronic acid. Flash column chromatography (DCM/MeOH 95:5). Obtained 0.050 g (75% of yield). ¹H NMR (500 MHz, DMSO-d₆) δ ppm 1.81-1.92 (m, 1H), 1.94-2.02 (m, 1H), 2.08-2.28 (m, 2H), 2.88 (m, 1H), 3.23-3.31 (m, 2H), 6.47 (d, J=8.85 Hz, 1H), 6.94 (d, J=8.39 Hz, 1H), 7.25 (d, J=7.63 Hz, 6H), 7.27-7.30 (m, 3H), 7.34-7.37 (m, 6H), 7.41 (t, J=8.08 Hz, 1H), 7.48 (m, 1H), 7.55 (d, J=8.08 Hz, 2H), 7.67 (s, 1H), 10.68 (s, 1H). HRMS (ESI) calcd for C38H31Cl2N4O2Na [M+H+Na]⁺667.1638, found 667.1664.

### N-[5-(5-Acetyl-2-fluorophenyl)-1-trityl-1H-indazol-3-yl]-6-oxopiperidine-3-carboxamide [(XI), R1=5-acetyl-2-fluorophenyl, R2=piperidin-2-one, R9=1,1′,1″-methanetriyltribenzene]

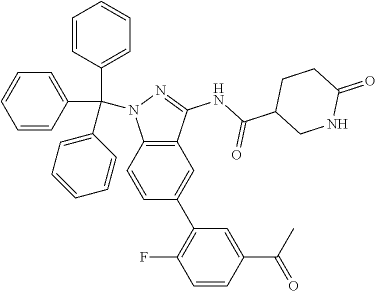 [SMILES: CC(=O)c1ccc(F)c(-c2ccc3c(c2)c(NC(=O)C2CCC(=O)NC2)nn3C(c2ccccc2)(c2ccccc2)c2ccccc2)c1]

Starting from N-(5-bromo-1-trityl-1H-indazol-3-yl)-6-oxopiperidine-3-carboxamide and (5-acetyl-2-fluorophenyl)boronic acid. Flash column chromatography (DCM/MeOH 95:5). Obtained 0.080 g (82% of yield). ¹H NMR (500 MHz, DMSO-d₆) δ ppm 1.87-1.94 (m, 1H), 1.99-2.05 (m, 1H), 2.13-2.26 (m, 2H), 2.60 (s, 3H), 2.91 (m, 1H), 3.29-3.31 (m, 2H), 6.49 (d, J=9.00 Hz, 1H), 7.25 (d, J=7.63 Hz, 6H), 7.28-7.31 (m, 4H), 7.34-7.37 (m, 6H), 7.44 (m, 1H), 7.50 (m, 1H), 7.93 (s, 1H), 7.95-8.04 (m, 2H), 10.71 (s, 1H). HRMS (ESI) calcd for C40H34FN4O3Na [M+H+Na]659.2429, found 659.2455.

### tert-Butyl (3R)-3-({5-[2-chloro-5-(methoxycarbonyl)phenyl]-1-trityl-1H-indazol-3-yl)carbamoyl)piperidine-1-carboxylate [(XI), R1=2-chloro-5-(methoxycarbonyl)phenyl, R2=tert-butyl piperidine-1-carboxylate, R9=1,1′,1″-methanetriyltribenzene]

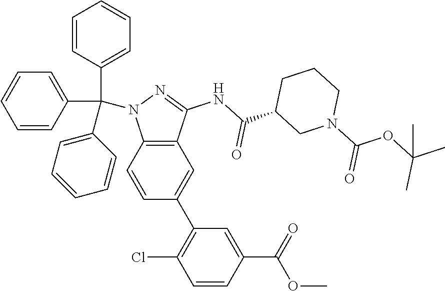 [SMILES: COC(=O)c1ccc(Cl)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and [2-chloro-5-(methoxycarbonyl)phenyl]boronic acid. Flash column chromatography (Hexane/EtOAc 70:30). Obtained 0.055 g (74% of yield). 1H NMR (500 MHz, DMSO-d₆) δ ppm 1.30-1.35 (m, 10H), 1.59-1.69 (m, 2H), 1.92-1.94 (m, 1H), 2.59 (m, 1H), 2.77-2.85 (m, 1H), 3.77 (d, J=13.42 Hz, 1H), 3.85 (s, 3H), 3.92-4.00 (m, 2H), 6.47 (d, J=8.85 Hz, 1H), 7.15 (d, J=9.91 Hz, 1H), 7.24 (d, J=7.63 Hz, 6H), 7.28-7.31 (m, 3H), 7.34-7.37 (m, 6H), 7.71 (d, J=8.24 Hz, 1H), 7.83-7.84 (m, 2H), 7.92 (dd, J=8.39, 2.14 Hz, 1H), 10.65 (s, 1H). HRMS (ESI) calcd for C45H44ClN4O5Na [M+H+Na]⁺777.2814, found 777.2816.

### tert-Butyl (3R)-3-{[5-(2,4-difluorophenyl)-1-trityl-1H-indazol-3-yl]carbamoyl}piperidine-1-carboxylate [(XI), R1=2,4-difluorophenyl, R2=tert-butyl piperidine-1-carboxylate, R9=1,1′,1″-methanetriyltribenzene]

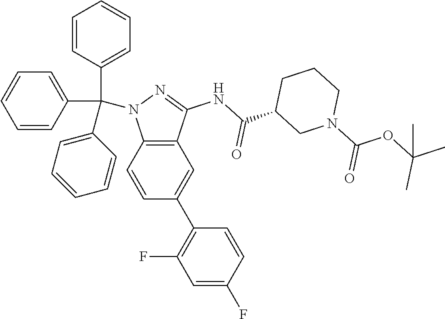 [SMILES: CC(C)(C)OC(=O)N1CCC[C@@H](C(=O)Nc2nn(C(c3ccccc3)(c3ccccc3)c3ccccc3)c3ccc(-c4ccc(F)cc4F)cc23)C1]

Starting from tert-butyl (3R)-3-[(5-bromo-1-trityl-1H-indazol-3-yl)carbamoyl]piperidine-1-carboxylate and (2,4-difluorophenyl)boronic acid. Flash column chromatography (Hexane/EtOAc 70:30). Obtained 0.098 g (51% of yield). LCMS (ESI) m/z 699 (M+H)⁺.

### tert-Butyl (3R)-3-({5-[4-methoxy-2-(trifluoromethyl)phenyl]-1-trityl-1H-indazol-3-yl}carbamoyl)piperidine-1-carboxylate [(XI), R1=4-methoxy-2-(trifluoromethyl)phenyl, R2=tert-butyl piperidine-1-carboxylate, R9=1,1′,1″-methanetriyltribenzene]

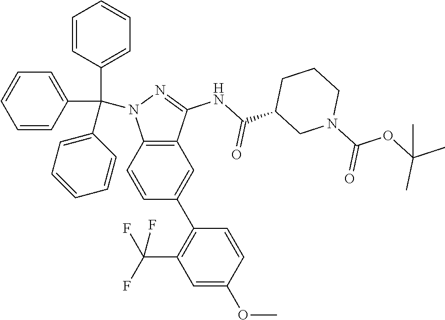 [SMILES: COc1ccc(-c2ccc3c(c2)c(NC(=O)[C@@H]2CCCN(C(=O)OC(C)(C)C)C2)nn3C(c2ccccc2)(c2ccccc2)c2ccccc2)c(C(F)(F)F)c1]

Starting from tert-butyl (3R)-3-[(5-bromo-1-trityl-1H-indazol-3-yl)carbamoyl]piperidine-1-carboxylate and [4-methoxy-2-(trifluoromethyl)phenyl]boronic acid. Flash column chromatography (Hexane/EtOAc 70:30). Obtained 0.062 g (83% of yield). ¹H NMR (500 MHz, DMSO-d₆) δ ppm 1.30-1.35 (m, 10H), 1.59-1.68 (m, 2H), 1.90-1.93 (m, 1H), 2.56 (m, 1H), 2.74-2.80 (m, 1H), 3.77 (d, J=13.27 Hz, 1H), 3.85 (s, 3H), 3.92-3.97 (m, 2H), 6.39 (d, J=8.85 Hz, 1H), 6.97 (d, J=8.69 Hz, 1H), 7.23-7.36 (m, 18H), 7.62 (s, 1H), 10.59 (s, 1H). HRMS (ESI) calcd for C45H44F3N4O4Na [M+H+Na]⁺783.3128, found 783.3122.

### tert-Butyl (3R)-3-{[5-(2-fluoro-5-methylphenyl)-1-trityl-1H-indazol-3-yl]carbamoyl}piperidine-1-carboxylate [(XI), R1=2-fluoro-5-methylphenyl, R2=tert-butyl piperidine-1-carboxylate, R9=

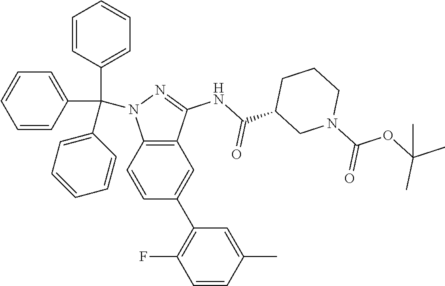 [SMILES: Cc1ccc(F)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and (2-fluoro-5-methylphenyl)boronic acid. Flash column chromatography (Hexane/EtOAc 70:30). Obtained 0.048 g (88% of yield). ¹H NMR (500 MHz, DMSO-d₆) δ ppm 1.31-1.37 (m, 10H), 1.62-1.71 (m, 2H), 1.94-1.97 (m, 1H), 2.30 (s, 3H), 2.59 (m, 1H), 2.74-2.81 (m, 1H), 3.81 (d, J=13.27 Hz, 1H), 3.93-4.03 (m, 2H), 6.44 (d, J=9.00 Hz, 1H), 7.14-7.20 (m, 4H), 7.22 (d, J=7.63 Hz, 6H), 7.28-7.30 (m, 3H), 7.33-7.36 (m, 6H), 7.86 (s, 1H), 10.62 (s, 1H). HRMS (ESI) calcd for C44H44FN4O3 [M+H]⁺ 695.3392, found 695.3387.

### tert-Butyl (3R)-3-{[5-(5-acetyl-2-chlorophenyl)-1-trityl-1H-indazol-3-yl]carbamoyl}piperidine-1-carboxylate [(XI), R1=5-acetyl-2-chlorophenyl, R2=tert-butyl piperidine-1-carboxylate, R9=methanetriyltribenzene]

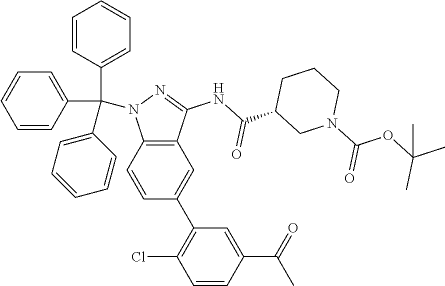 [SMILES: CC(=O)c1ccc(Cl)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and (5-acetyl-2-chlorophenyl)boronic acid. Flash column chromatography (Hexane/EtOAc 80:20). Obtained 0.061 g (84% of yield). ¹H NMR (500 MHz, DMSO-d₆) δ ppm 1.29-1.35 (m, 10H), 1.51-1.75 (m, 2H), 1.86-1.98 (m, 1H), 2.56-2.60 (m, 4H), 2.74-2.82 (m, 1H), 3.77 (d, J=12.81 Hz, 1H), 3.88-3.97 (m, 2H), 6.47 (d, J=9.00 Hz, 1H), 7.16 (d, J=9.00 Hz, 1H), 7.24 (d, J=7.63 Hz, 6H), 7.28-7.31 (m, 3H), 7.34-7.37 (m, 6H), 7.70 (d, J=8.39 Hz, 1H), 7.83 (s, 1H), 7.87 (s, 1H), 7.91 (dd, J=8.39, 2.14 Hz, 1H), 10.64 (s, 1H). HRMS (ESI) calcd for C45H44ClN4O4Na [M+H+Na]⁺761.2865, found 761.2868.

### tert-Butyl (3R)-3-{[5-(5-amino-2-chlorophenyl)-1-trityl-1H-indazol-3-yl]carbamoyl}piperidine-1-carboxylate [(XI), R1=5-amino-2-chlorophenyl, R2=tert-butyl piperidine-1-carboxylate, R9=methanetriyltribenzene]

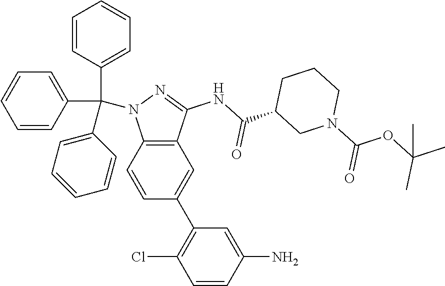 [SMILES: CC(C)(C)OC(=O)N1CCC[C@@H](C(=O)Nc2nn(C(c3ccccc3)(c3ccccc3)c3ccccc3)c3ccc(-c4cc(N)ccc4Cl)cc23)C1]

Starting from tert-butyl (3R)-3-[(5-bromo-1-trityl-1H-indazol-3-yl)carbamoyl]piperidine-1-carboxylate and (5-amino-2-chlorophenyl)boronic acid. Flash column chromatography (Hexane/EtOAc 70:30). Obtained 0.045 g (74% of yield). 1H NMR (500 MHz, DMSO-d₆) δ ppm 1.30-1.37 (m, 10H), 1.59-1.69 (m, 2H), 1.94-1.96 (m, 1H), 2.57 (m, 1H), 2.72-2.78 (m, 1H), 3.82 (d, J=13.73 Hz, 1H), 3.91-3.98 (m, 2H), 5.28 (s, 2H), 6.40 (d, J=9.00 Hz, 1H), 6.48-6.56 (m, 2H), 7.05 (d, J=9.00 Hz, 1H), 7.09 (d, J=8.24 Hz, 1H), 7.23 (d, J=7.63 Hz, 6H), 7.27-7.30 (m, 3H), 7.33-7.36 (m, 6H), 7.71 (s, 1H), 10.60 (s, 1H). HRMS (ESI) calcd for C43H43ClN5O3Na [M+H+Na]⁺734.2868, found 734.2874.

### tert-Butyl (3R)-3-{[5-(2,5-difluorophenyl)-1-trityl-1H-indazol-3-yl]carbamoyl}piperidine-1-carboxylate [(XI), R1=2,5-difluorophenyl, R2=tert-butyl piperidine-1-carboxylate, R9=1,1′,1″-methanetriyltribenzene]

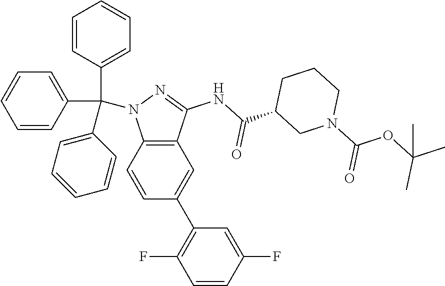 [SMILES: CC(C)(C)OC(=O)N1CCC[C@@H](C(=O)Nc2nn(C(c3ccccc3)(c3ccccc3)c3ccccc3)c3ccc(-c4cc(F)ccc4F)cc23)C1]

Starting from tert-butyl (3R)-3-[(5-bromo-1-trityl-1H-indazol-3-yl)carbamoyl]piperidine-1-carboxylate and (2,5-difluorophenyl)boronic acid. Flash column chromatography (Hexane/EtOAc 80:20). Obtained 0.071 g (59% of yield). ¹H NMR (500 MHz, DMSO-d₆) δ ppm 1.31-1.37 (m, 10H), 1.61-1.71 (m, 2H), 1.94-1.99 (m, 1H), 2.59 (m, 1H), 2.75-2.82 (m, 1H), 3.80 (d, J=12.81 Hz, 1H), 3.93-4.00 (m, 2H), 6.46 (d, J=8.85 Hz, 1H), 7.18-7.36 (m, 19H), 7.92 (s, 1H), 10.65 (s, 1H). HRMS (ESI) calcd for C43H41F2N4O3 [M+H]⁺ 699.3141, found 699.3129.

### tert-Butyl (3R)-3-({5-[4-methyl-2-(trifluoromethyl)phenyl]-1-trityl-1H-indazol-3-yl}carbamoyl)piperidine-1-carboxylate [(XI), R1=4-methyl-2-(trifluoromethyl)phenyl, R2=tert-butyl piperidine-1-carboxylate, R9=1,1′,1″-methanetriyltribenzene]

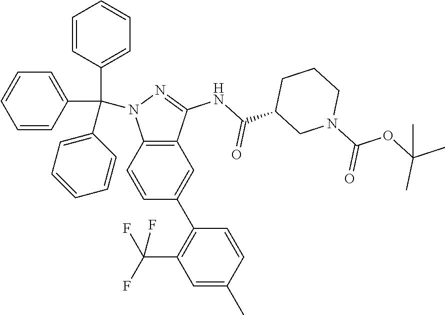 [SMILES: Cc1ccc(-c2ccc3c(c2)c(NC(=O)[C@@H]2CCCN(C(=O)OC(C)(C)C)C2)nn3C(c2ccccc2)(c2ccccc2)c2ccccc2)c(C(F)(F)F)c1]

Starting from tert-butyl (3R)-3-[(5-bromo-1-trityl-1H-indazol-3-yl)carbamoyl]piperidine-1-carboxylate and [4-methyl-2-(trifluoromethyl)phenyl]boronic acid. Flash column chromatography (Hexane/EtOAc 80:20). Obtained 0.047 g (65% of yield). ¹H NMR (500 MHz, DMSO-d₆) δ ppm 1.29-1.35 (m, 10H), 1.55-1.68 (m, 2H), 1.89-1.93 (m, 1H), 2.41 (s, 3H), 2.56 (m, 1H), 2.70-2.79 (m, 1H), 3.78 (d, J=14.03 Hz, 1H), 3.87-3.98 (m, 1H), 6.40 (d, J=8.85 Hz, 1H), 6.98 (d, J=9.30 Hz, 1H), 7.23 (d, J=7.63 Hz, 6H), 7.27-7.30 (m, 3H), 7.33-7.36 (m, 6H), 7.40 (d, J=0.91 Hz, 1H), 7.46 (d, J=7.93 Hz, 1H), 7.58 (s, 1H), 7.66 (s, 1H), 10.60 (s, 1H). HRMS (ESI) calcd for C45H44F3N4O3Na [M+H+Na]⁺767.3179, found 767.3188.

### tert-Butyl (3R)-3-({5-[2-chloro-5-(methoxycarbonyl)phenyl]-1-trityl-1H-indazol-3-yl}carbamoyl)pyrrolidine-1-carboxylate [(XI), R1=2-chloro-5-(methoxycarbonyl)phenyl, R2=tert-butyl pyrrolidine-1-carboxylate, R9=1,1′,1″-methanetriyltribenzene]

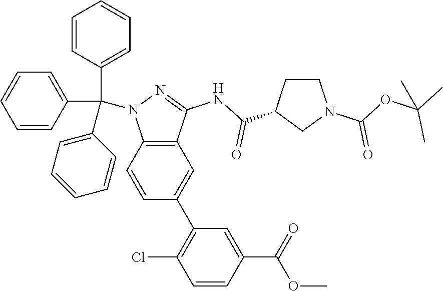 [SMILES: COC(=O)c1ccc(Cl)c(-c2ccc3c(c2)c(NC(=O)[C@@H]2CCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]pyrrolidine-1-carboxylate and [2-chloro-5-(methoxycarbonyl)phenyl]boronic acid. Flash column chromatography (Hexane/EtOAc 70:30). Obtained 0.054 g (73% of yield). ¹H NMR (500 MHz, DMSO-d₆) δ ppm 1.36 (s, 9H), 2.05-2.17 (m, 2H), 3.12-3.28 (m, 2H), 3.37-3.41 (m, 2H), 3.51 (d, J=8.54 Hz, 1H), 3.85 (s, 3H), 6.48 (d, J=9.00 Hz, 1H), 7.15 (d, J=8.54 Hz, 1H), 7.24 (d, J=7.63 Hz, 6H), 7.28-7.31 (m, 3H), 7.34-7.37 (m, 6H), 7.71 (d, J=8.39 Hz, 1H), 7.80-7.87 (m, 2H), 7.92 (dd, J=8.24, 2.14 Hz, 1H), 10.71 (d, J=6.71 Hz, 1H). HRMS (ESI) calcd for C44H42ClN4O5Na [M+H+Na]⁺763.2657, found 763.2645. tert-Butyl (3R)-3-{[5-(5-acetyl-2-chlorophenyl)-1-trityl-1H-indazol-3-yl]carbamoyl}pyrrolidine-1-carboxylate [(XI), R1=5-acetyl-2-chlorophenyl, R2=tert-butyl pyrrolidine-1-carboxylate, R9=methanetriyltribenzene]

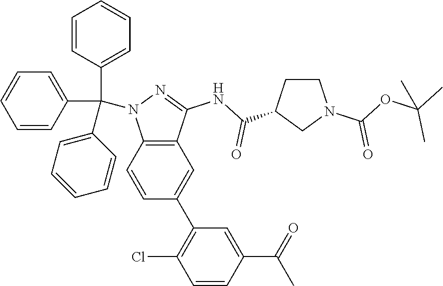 [SMILES: CC(=O)c1ccc(Cl)c(-c2ccc3c(c2)c(NC(=O)[C@@H]2CCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]pyrrolidine-1-carboxylate and (5-acetyl-2-chlorophenyl)boronic acid. Flash column chromatography (Hexane/EtOAc 70:30). Obtained 0.047 g (65% of yield). ¹H NMR (500 MHz, DMSO-d₆) δ ppm 1.36 (s, 9H), 2.01-2.15 (m, 2H), 2.59 (s, 3H), 3.15-3.29 (m, 2H), 3.36-3.43 (m, 2H), 3.48-3.52 (m, 1H), 6.48 (d, J=8.85 Hz, 1H), 7.17 (d, J=9.00 Hz, 1H), 7.24 (d, J=7.63 Hz, 6H), 7.28-7.31 (m, 3H), 7.34-7.37 (m, 6H), 7.70 (d, J=8.39 Hz, 1H), 7.79-7.88 (m, 2H), 7.91 (dd, J=8.39, 2.14 Hz, 1H), 10.71 (d, J=5.64 Hz, 1H). HRMS (ESI) calcd for C44H42ClN4O4Na [M+H+Na]⁺747.2708, found 747.2712.

### tert-Butyl (3R)-3-({5-[2-chloro-5-(methylcarbamoyl)phenyl]-1-trityl-1H-indazol-3-yl}carbamoyl)pyrrolidine-1-carboxylate [(XI), R1=2-chloro-5-(methylcarbamoyl)phenyl, R2=tert-butyl pyrrolidine-1-carboxylate, R9=1,1′,1″-methanetriyltribenzene]

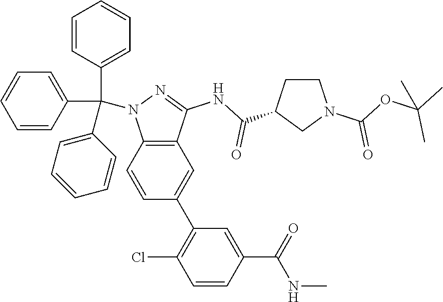 [SMILES: CNC(=O)c1ccc(Cl)c(-c2ccc3c(c2)c(NC(=O)[C@@H]2CCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]pyrrolidine-1-carboxylate and [2-chloro-5-(methylcarbamoyl)phenyl]boronic acid. Flash column chromatography (Hexane/EtOAc 30:70). Obtained 0.045 g (61% of yield). ¹H NMR (500 MHz, DMSO-d₆) δ ppm 1.36 (s, 9H), 2.03-2.13 (m, 2H), 2.75 (d, J=4.58 Hz, 3H), 3.13-3.29 (m, 2H), 3.37-3.40 (m, 1H), 3.43-3.57 (m, 1H), 6.48 (d, J=8.85 Hz, 1H), 7.16 (d, J=8.85 Hz, 1H), 7.24 (d, J=7.78 Hz, 6H), 7.28-7.31 (m, 3H), 7.34-7.37 (m, 6H), 7.63 (d, J=8.85 Hz, 1H), 7.80-7.81 (m, 3H), 8.55 (d, J=4.57 Hz, 1H), 10.70 (d, J=6.86 Hz, 1H). HRMS (ESI) calcd for C44H43ClN5O4Na [M+H+Na]⁺762.2817, found 762.2827.

### tert-Butyl (3R)-3-({5-[2-cyano-5-(methoxycarbonyl)phenyl]-1-trityl-1H-indazol-3-yl)carbamoyl)piperidine-1-carboxylate [(XI), R1=2-cyano-5-(methoxycarbonyl)phenyl, R2=tert-butyl piperidine-1-carboxylate, R9=1,1′,1″-methanetriyltribenzene]

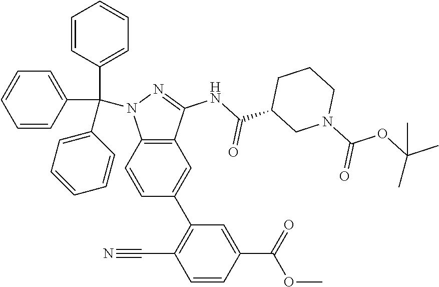 [SMILES: COC(=O)c1ccc(C#N)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and methyl 4-cyano-3-(4,4,5,5-tetramethyl-1,3,2-dioxaborolan-2-yl)benzoate. Flash column chromatography (Hexane/EtOAc 80:20). Obtained 0.160 g (84% of yield). LCMS (ESI) m/z 746 (M+H)⁺.

### tert-Butyl (3R)-3-{[5-(2-chloro-5-hydroxyphenyl)-1-trityl-1H-indazol-3-yl]carbamoyl}piperidine-1-carboxylate [(XI), R1=2-chloro-5-hydroxyphenyl, R2=tert-butyl piperidine-1-carboxylate, R9=methanetriyltribenzene]

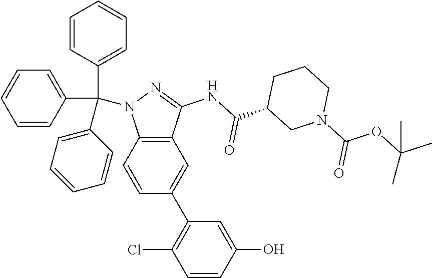 [SMILES: CC(C)(C)OC(=O)N1CCC[C@@H](C(=O)Nc2nn(C(c3ccccc3)(c3ccccc3)c3ccccc3)c3ccc(-c4cc(O)ccc4Cl)cc23)C1]

Starting from tert-butyl (3R)-3-[(5-bromo-1-trityl-1H-indazol-3-yl)carbamoyl]piperidine-1-carboxylate and (2-chloro-5-hydroxypheny)lboronic acid. Flash column chromatography (Hexane/EtOAc 70:30). Obtained 0.045 g (58% of yield). ¹H NMR (500 MHz, DMSO-d₆) δ ppm 1.30-1.37 (m, 10H), 1.57-1.72 (m, 2H), 1.95 (d, J=12.81 Hz, 1H), 2.58 (m, 1H), 2.72-2.80 (m, 1H), 3.82 (d, J=13.12 Hz, 1H), 3.97-4.02 (m, 2H), 6.42 (d, J=9.00 Hz, 1H), 6.71 (d, J=2.75 Hz, 1H), 6.75 (dd, J=8.62, 2.97 Hz, 1H), 7.10 (d, J=8.85 Hz, 1H), 7.23 (d, J=7.47 Hz, 6H), 7.27-7.30 (m, 4H), 7.33-7.36 (m, 6H), 7.74 (s, 1H), 9.77 (s, 1H), 10.63 (s, 1H). HRMS (ESI) calcd for C43H42ClN4O4 [M+H]⁺ 713.2889, found 713.2894.

### tert-Butyl (3R)-3-({5-[2,4-dichloro-5-(methoxycarbonyl)phenyl]-1-trityl-1H-indazol-3-yl)carbamoyl)piperidine-1-carboxylate [(XI), R1=2,4-dichloro-5-(methoxycarbonyl)phenyl, R2=tert-butyl piperidine-1-carboxylate, R9=1,1′,1″-methanetriyltribenzene]

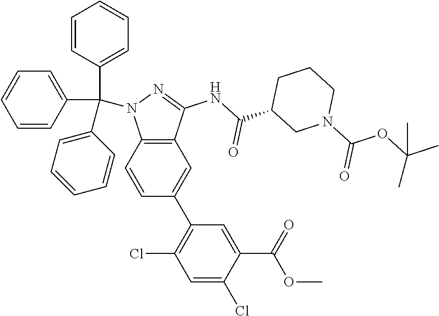 [SMILES: COC(=O)c1cc(-c2ccc3c(c2)c(NC(=O)[C@@H]2CCCN(C(=O)OC(C)(C)C)C2)nn3C(c2ccccc2)(c2ccccc2)c2ccccc2)c(Cl)cc1Cl]

Starting from tert-butyl (3R)-3-[(5-bromo-1-trityl-1H-indazol-3-yl)carbamoyl]piperidine-1-carboxylate and methyl 2,4-dichloro-5-(4,4,5,5-tetramethyl-1,3,2-dioxaborolan-2-yl)benzoate. Flash column chromatography (Hexane/EtOAc 70:30). Obtained 0.030 g (55% of yield). LCMS (ESI) m/z 789 (M+H)⁺.

### tert-Butyl (3R)-3-{[5-(2-chloro-5-propanoylphenyl)-1-trityl-1H-indazol-3-yl]carbamoyl}piperidine-1-carboxylate [(XI), R1=2-chloro-5-propanoylphenyl, R2=tert-butyl piperidine-1-carboxylate, R9=methanetriyltribenzene]

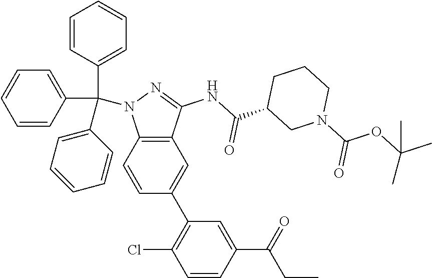 [SMILES: CCC(=O)c1ccc(Cl)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and (2-chloro-5-propanoylphenyl)boronic acid. Flash column chromatography (Hexane/EtOAc 70:30). Obtained 0.023 g (50% of yield). LCMS (ESI) m/z 753 (M+H)⁺.

### tert-Butyl (3R)-3-({5-[2-chloro-4-(trifluoromethyl)phenyl]-1-trityl-1H-indazol-3-yl)carbamoyl)piperidine-1-carboxylate [(XI), R1=2-chloro-4-(trifluoromethyl)phenyl, R2=tert-butyl piperidine-1-carboxylate, R9=1,1′,1″-methanetriyltribenzene]

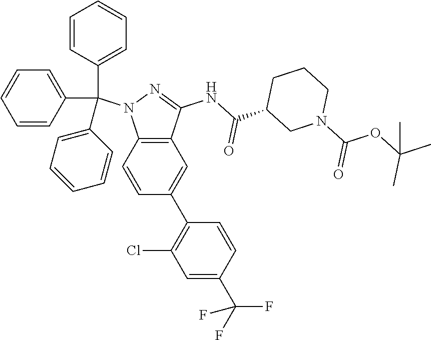 [SMILES: CC(C)(C)OC(=O)N1CCC[C@@H](C(=O)Nc2nn(C(c3ccccc3)(c3ccccc3)c3ccccc3)c3ccc(-c4ccc(C(F)(F)F)cc4Cl)cc23)C1]

Starting from tert-butyl (3R)-3-[(5-bromo-1-trityl-1H-indazol-3-yl)carbamoyl]piperidine-1-carboxylate and [2-chloro-4-(trifluoromethyl)phenyl]boronic acid. Flash column chromatography (Hexane/EtOAc 80:20). Obtained 0.058 g (85% of yield). ¹H NMR (500 MHz, DMSO-d₆) δ ppm 1.29-1.38 (m, 10H), 1.56-1.73 (m, 2H), 1.93 (d, J=10.98 Hz, 1H), 2.59 (m, 1H), 2.75-2.84 (m, 1H), 3.77 (d, J=12.20 Hz, 1H), 3.90-4.00 (m, 2H), 6.47 (d, J=8.85 Hz, 1H), 7.18 (d, J=8.85 Hz, 1H), 7.24 (d, J=7.47 Hz, 6H), 7.28-7.31 (m, 3H), 7.34-7.37 (m, 6H), 7.60 (d, J=8.08 Hz, 1H), 7.76 (d, J=8.08 Hz, 1H), 7.85 (s, 1H), 7.95 (s, 1H), 10.66 (s, 1H). HRMS (ESI) calcd for C44H41ClF3N4O3Na [M+H+Na]⁺787.2633, found 787.2635.

### tert-Butyl (3R)-3-({5-[4-chloro-2-(trifluoromethyl)phenyl]-1-trityl-1H-indazol-3-yl)carbamoyl)piperidine-1-carboxylate [(XI), R1=4-chloro-2-(trifluoromethyl)phenyl, R2=tert-butyl piperidine-1-carboxylate, R9=1,1′,1″-methanetriyltribenzene]

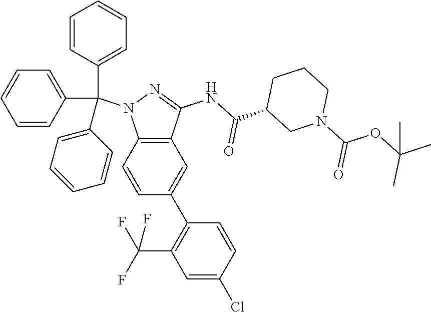 [SMILES: CC(C)(C)OC(=O)N1CCC[C@@H](C(=O)Nc2nn(C(c3ccccc3)(c3ccccc3)c3ccccc3)c3ccc(-c4ccc(Cl)cc4C(F)(F)F)cc23)C1]

Starting from tert-butyl (3R)-3-[(5-bromo-1-trityl-1H-indazol-3-yl)carbamoyl]piperidine-1-carboxylate and [4-chloro-2-(trifluoromethyl)phenyl]boronic acid. Flash column chromatography (Hexane/EtOAc 80:20). Obtained 0.062 g (90% of yield). ¹H NMR (500 MHz, DMSO-d₆) δ ppm 1.26-1.35 (m, 10H), 1.57-1.69 (m, 2H), 1.92 (d, J=10.52 Hz, 1H), 2.57 (m, 1H), 2.72-2.82 (m, 1H), 3.77 (d, J=13.12 Hz, 1H), 3.87-3.98 (m, 2H), 6.42 (d, J=9.00 Hz, 1H), 7.01 (d, J=8.85 Hz, 1H), 7.23 (d, J=7.63 Hz, 6H), 7.27-7.30 (m, 3H), 7.33-7.36 (m, 6H), 7.40 (d, J=8.39 Hz, 1H), 7.69 (s, 1H), 7.76 (dd, J=8.24, 1.98 Hz, 1H), 7.86 (d, J=1.98 Hz, 1H), 10.63 (s, 1H). HRMS (ESI) calcd for C44H41ClF3N4O3Na [M+H+Na]⁺787.2633, found 787.2650.

### tert-Butyl (3R)-3-({5-[5-fluoro-2-(trifluoromethyl)phenyl]-1-trityl-1H-indazol-3-yl)carbamoyl)piperidine-1-carboxylate [(XI), R1=5-fluoro-2-(trifluoromethyl)phenyl, R2=tert-butyl piperidine-1-carboxylate, R9=1,1′,1″-methanetriyltribenzene]

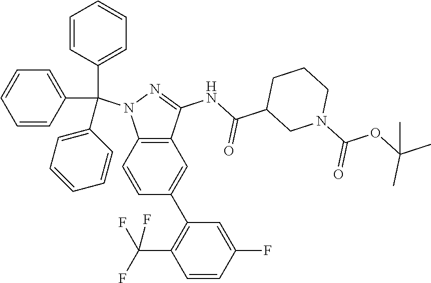 [SMILES: CC(C)(C)OC(=O)N1CCCC(C(=O)Nc2nn(C(c3ccccc3)(c3ccccc3)c3ccccc3)c3ccc(-c4cc(F)ccc4C(F)(F)F)cc23)C1]

Starting from tert-butyl (3R)-3-[(5-bromo-1-trityl-1H-indazol-3-yl)carbamoyl]piperidine-1-carboxylate and [5-fluoro-2-(trifluoromethyl)phenyl]boronic acid. Flash column chromatography (Hexane/EtOAc 80:20). Obtained 0.060 g (89% of yield). ¹H NMR (500 MHz, DMSO-d₆) δ ppm 1.29-1.38 (m, 10H), 1.57-1.69 (d, 2H), 1.90 (d, J=13.27 Hz, 1H), 2.57 (m, 1H), 2.73-2.80 (m, 1H), 3.77 (d, J=11.59 Hz, 1H), 3.89-3.99 (m, 2H), 6.42 (d, J=9.00 Hz, 1H), 7.04 (d, J=9.00 Hz, 1H), 7.18 (d, J=7.02 Hz, 1H), 7.23 (d, J=7.47 Hz, 6H), 7.28-7.31 (m, 3H), 7.33-7.46 (m, 6H), 7.43 (m, 1H), 7.72 (s, 1H), 7.88 (dd, J=8.85, 5.49 Hz, 1H), 10.63 (s, 1H). HRMS (ESI) calcd for C44H41F4N4O3 [M+H]⁺ 749.3110, found 749.3121.

### tert-Butyl (3R)-3-{[5-(2-chlorophenyl)-1-trityl-1H-indazol-3-yl]carbamoyl}piperidine-1-carboxylate [(XI), R1=2-chlorophenyl, R2=tert-butyl piperidine-1-carboxylate, R9=1,1′,1″-methanetriyltribenzene]

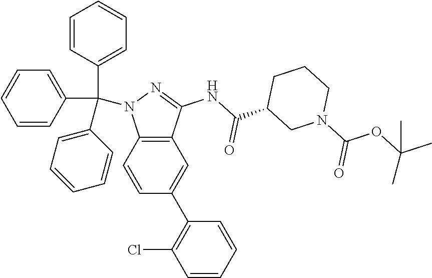 [SMILES: CC(C)(C)OC(=O)N1CCC[C@@H](C(=O)Nc2nn(C(c3ccccc3)(c3ccccc3)c3ccccc3)c3ccc(-c4ccccc4Cl)cc23)C1]

Starting from tert-butyl (3R)-3-[(5-bromo-1-trityl-1H-indazol-3-yl)carbamoyl]piperidine-1-carboxylate and (2-chlorophenyl)boronic acid. Flash column chromatography (Hexane/EtOAc 70:30). Obtained 0.037 g (76% of yield). LCMS (ESI) m/z 697 (M+H)⁺.

### tert-Butyl (3R)-3-({5-[2-chloro-5-(propan-2-yl)phenyl]-1-trityl-1H-indazol-3-yl}carbamoyl)piperidine-1-carboxylate [(XI), R1=2-chloro-5-(propan-2-yl)phenyl, R2=tert-butyl piperidine-1-carboxylate, R9=1,1′,1″-methanetriyltribenzene]

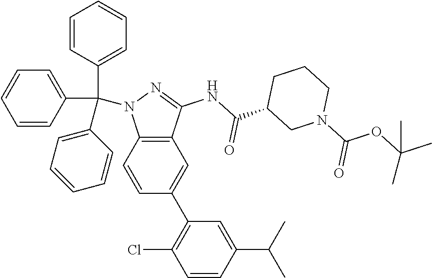 [SMILES: CC(C)c1ccc(Cl)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-[(5-bromo-1-trityl-1H-indazol-3-yl)carbamoyl]piperidine-1-carboxylate and [2-chloro-5-(propan-2-yl)phenyl]boronic acid. Flash column chromatography (Hexane/EtOAc 75:25). Obtained 0.042 g (81% of yield). ¹H NMR (500 MHz, DMSO-d₆) δ ppm 1.18 (d, J=7.02 Hz, 6H), 1.30-1.37 (m, 10H), 1.60-1.70 (m, 2H), 1.90-1.97 (m, 1H), 2.58 (m, 1H), 2.76-2.83 (m, 1H), 2.91 (m, 1H), 3.77 (d, J=13.42 Hz, 1H), 3.90-4.00 (m, 1H), 6.44 (d, J=8.85 Hz, 1H), 7.11 (d, J=9.00 Hz, 1H), 7.19 (s, 1H), 7.22-7.36 (m, 16H), 7.43 (d, J=8.24 Hz, 1H), 7.75 (s, 1H), 10.61 (s, 1H). HRMS (ESI) calcd for C46H48ClN4O3 [M+H]⁺ 739.3410, found 739.3405.

### tert-Butyl (3R)-3-[(5-phenyl-1-trityl-1H-indazol-3-yl)carbamoyl]piperidine-1-carboxylate [(XI), R1=phenyl, R2=tert-butyl piperidine-1-carboxylate, R9=1,1′,1″-methanetriyltribenzene]

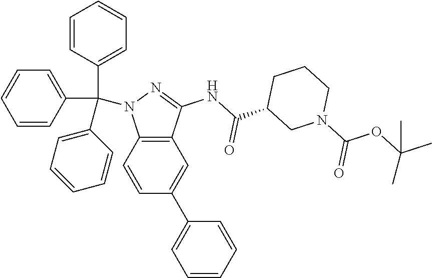 [SMILES: CC(C)(C)OC(=O)N1CCC[C@@H](C(=O)Nc2nn(C(c3ccccc3)(c3ccccc3)c3ccccc3)c3ccc(-c4ccccc4)cc23)C1]

Starting from tert-butyl (3R)-3-[(5-bromo-1-trityl-1H-indazol-3-yl)carbamoyl]piperidine-1-carboxylate and phenylboronic acid. Flash column chromatography (Hexane/EtOAc 75:25). Obtained 0.041 g (89% of yield). ¹H NMR (500 MHz, DMSO-d₆) δ ppm 1.32-1.38 (m, 10H), 1.63-1.72 (m, 2H), 1.95-2.04 (m, 1H), 2.60 (m, 1H), 2.75-2.82 (m, 1H), 3.83 (d, J=12.96 Hz, 1H), 3.92-4.05 (m, 2H), 6.44 (d, J=9.00 Hz, 1H), 7.22 (d, J=7.63 Hz, 6H), 7.27-7.37 (m, 11H), 7.43 (t, J=7.70 Hz, 2H), 7.55-7.56 (m, 2H), 7.94 (s, 1H), 10.62 (s, 1H). HRMS (ESI) calcd for C43H43N4O3 [M+H]⁺ 663.3330, found 663.3345.

### tert-Butyl (3R)-3-({5-[2,4-difluoro-5-(methoxycarbonyl)phenyl]-1-trityl-1H-indazol-3-yl)carbamoyl)piperidine-1-carboxylate [(XI), R1=2,4-difluoro-5-(methoxycarbonyl)phenyl, R2=tert-butyl piperidine-1-carboxylate, R9=1,1′,1″-methanetriyltribenzene]

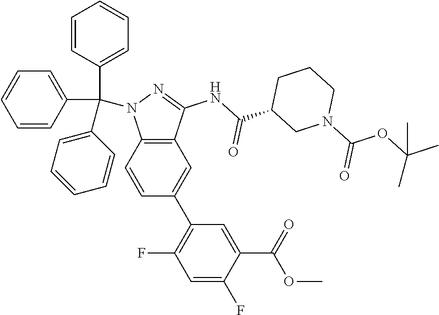 [SMILES: COC(=O)c1cc(-c2ccc3c(c2)c(NC(=O)[C@@H]2CCCN(C(=O)OC(C)(C)C)C2)nn3C(c2ccccc2)(c2ccccc2)c2ccccc2)c(F)cc1F]

Starting from tert-butyl (3R)-3-[(5-bromo-1-trityl-1H-indazol-3-yl)carbamoyl]piperidine-1-carboxylate and methyl 2,4-difluoro-5-(4,4,5,5-tetramethyl-1,3,2-dioxaborolan-2-yl)benzoate. Flash column chromatography (Hexane/EtOAc 70:30). Obtained 0.036 g (68% of yield). ¹H NMR (500 MHz, DMSO-d₆) δ ppm 1.31-1.39 (m, 10H), 1.61-1.71 (m, 2H), 1.94-1.98 (m, 1H), 2.60 (m, 1H), 2.75-2.84 (m, 1H), 3.79 (d, J=12.05 Hz, 1H), 3.85 (s, 3H), 3.92-4.04 (m, 2H), 6.48 (d, J=9.15 Hz, 1H), 7.22 (d, J=7.63 Hz, 6H), 7.28-7.31 (m, 4H), 7.33-7.36 (m, 6H), 7.55 (t, J=10.68 Hz, 1H), 7.86-7.96 (m, 2H), 10.66 (s, 1H). HRMS (ESI) calcd for C45H43F2N4O5 [M+H]⁺ 757.3196, found 757.3211.

### tert-Butyl (3R)-3-{[5-(5-tert-butyl-2-chlorophenyl)-1-trityl-1H-indazol-3-yl]carbamoyl}piperidine-1-carboxylate [(XI), R1=5-tert-butyl-2-chlorophenyl, R2=tert-butyl piperidine-1-carboxylate, R9=1,1′,1-methanetriyltribenzene]

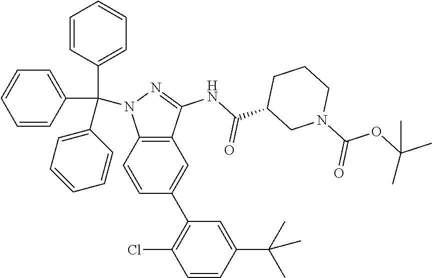 [SMILES: CC(C)(C)OC(=O)N1CCC[C@@H](C(=O)Nc2nn(C(c3ccccc3)(c3ccccc3)c3ccccc3)c3ccc(-c4cc(C(C)(C)C)ccc4Cl)cc23)C1]

Starting from tert-butyl (3R)-3-[(5-bromo-1-trityl-1H-indazol-3-yl)carbamoyl]piperidine-1-carboxylate and (5-tert-butyl-2-chlorophenyl)boronic acid. Flash column chromatography (Hexane/EtOAc 75:25). Obtained 0.047 g (80% of yield). ¹H NMR (500 MHz, DMSO-d₆) δ ppm 1.27 (s, 9H), 1.32-1.37 (m, 10H), 1.59-1.70 (m, 2H), 1.91-1.96 (m, 1H), 2.58 (m, 1H), 2.74-2.83 (m, 1H), 3.76 (d, J=12.81 Hz, 1H), 3.91-4.00 (m, 2H), 6.43 (d, J=9.00 Hz, 1H), 7.11 (d, J=8.24 Hz, 1H), 7.22-7.38 (m, 16H), 7.39 (d, J=2.29 Hz, 1H), 7.43 (d, J=8.39 Hz, 1H), 7.74 (s, 1H), 10.61 (s, 1H). HRMS (ESI) calcd for C47H50ClN4O3 [M+H]⁺ 753.3566, found 753.3578.

### tert-Butyl (3R)-3-{[5-(2-chloro-5-fluorophenyl)-1-trityl-1H-indazol-3-yl]carbamoyl}piperidine-1-carboxylate [(XI), R1=2-chloro-5-fluorophenyl, R2=tert-butyl piperidine-1-carboxylate, R9=1,1′,1-methanetriyltribenzene]

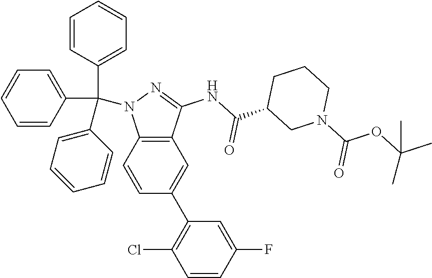 [SMILES: CC(C)(C)OC(=O)N1CCC[C@@H](C(=O)Nc2nn(C(c3ccccc3)(c3ccccc3)c3ccccc3)c3ccc(-c4cc(F)ccc4Cl)cc23)C1]

Starting from tert-butyl (3R)-3-[(5-bromo-1-trityl-1H-indazol-3-yl)carbamoyl]piperidine-1-carboxylate and (2-chloro-5-fluorophenyl)boronic acid. Flash column chromatography (Hexane/EtOAc 85:15). Obtained 0.048 g (96% of yield). ¹H NMR (500 MHz, DMSO-d₆) δ ppm 1.30-1.36 (m, 10H), 1.60-1.70 (m, 2H), 1.93-1.96 (m, 1H), 2.58 (m, 1H), 2.75-2.84 (m, 1H), 3.78 (d, J=13.12 Hz, 1H), 3.89-3.99 (m, 2H), 6.45 (d, J=8.85 Hz, 1H), 7.15 (d, J=8.69 Hz, 1H), 7.24 (d, J=8.24 Hz, 6H), 7.27-7.31 (m, 5H), 7.33-7.36 (m, 6H), 7.58 (dd, J=8.62, 5.26 Hz, 1H), 7.82 (s, 1H), 10.64 (s, 1H). HRMS (ESI) calcd for C43H41ClFN4O3 [M+H]⁺ 715.2846, found 715.2831.

### tert-Butyl (3R)-3-{[5-(2,5-dicyanophenyl)-1-trityl-1H-indazol-3-yl]carbamoyl}piperidine-1-carboxylate [(XI), R1=2,5-dicyanophenyl, R2=tert-butyl piperidine-1-carboxylate, R9=1,1′,1″-methanetriyltribenzene]

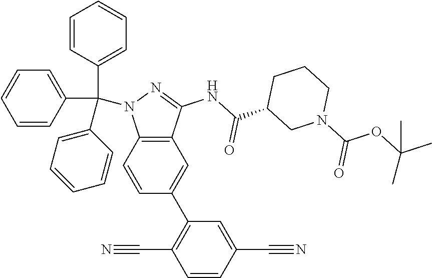 [SMILES: CC(C)(C)OC(=O)N1CCC[C@@H](C(=O)Nc2nn(C(c3ccccc3)(c3ccccc3)c3ccccc3)c3ccc(-c4cc(C#N)ccc4C#N)cc23)C1]

Starting from tert-butyl (3R)-3-[(5-bromo-1-trityl-1H-indazol-3-yl)carbamoyl]piperidine-1-carboxylate and 2-(4,4,5,5-tetramethyl-1,3,2-dioxaborolan-2-yl)benzene-1,4-dicarbonitrile. Flash column chromatography (Hexane/EtOAc 70:30). Obtained 0.013 g (22% of yield). LCMS (ESI) m/z 713 (M+H)⁺.

### N-[5-(2-Chloro-6-fluorophenyl)-1-trityl-1H-indazol-3-yl]-1-methylpiperidine-4-carboxamide [(XI), R1=2-chloro-6-fluorophenyl, R2=1-methylpiperidine, R9=1,1′,1″-methanetriyltribenzene]

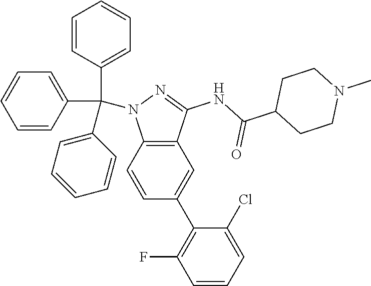 [SMILES: CN1CCC(C(=O)Nc2nn(C(c3ccccc3)(c3ccccc3)c3ccccc3)c3ccc(-c4c(F)cccc4Cl)cc23)CC1]

Starting from N-(5-bromo-1-trityl-1H-indazol-3-yl)-1-methylpiperidine-4-carboxamide and (2-chloro-6-fluorophenyl)boronic acid. Flash column chromatography (DCM/MeOH 80:20). Obtained 0.053 g (56% of yield). ¹H NMR (500 MHz, DMSO-d₆) δ ppm 1.64-1.75 (m, 2H), 1.82-1.89 (m, 2H), 2.33-2.44 (m, 4H), 2.98-3.09 (m, 2H), 6.48 (d, J=8.85 Hz, 1H), 7.03 (d, J=8.69 Hz, 1H), 7.19-7.32 (m, 10H), 7.32-7.39 (m, 6H), 7.40-7.47 (m, 2H), 7.74 (s, 1H), 10.57 (br. s., 1H). HRMS (ESI) calcd for C39H35ClFN4O [M+H]⁺ 629.2478, found 629.2497.

### 1-Butyl-N-[5-(2-chloro-6-fluorophenyl)-1-trityl-1H-indazol-3-yl]piperidine-4-carboxamide [(XI), R1=2-chloro-6-fluorophenyl, R2=1-butylpiperidine, R9=1,1′,1″-methanetriyltribenzene]

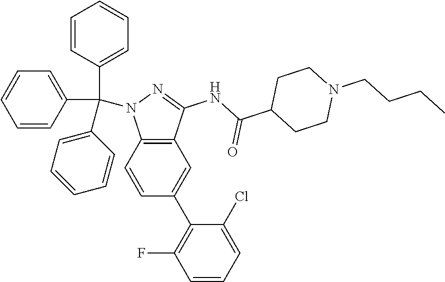 [SMILES: CCCCN1CCC(C(=O)Nc2nn(C(c3ccccc3)(c3ccccc3)c3ccccc3)c3ccc(-c4c(F)cccc4Cl)cc23)CC1]

Starting from N-(5-bromo-1-trityl-1H-indazol-3-yl)-1-butylpiperidine-4-carboxamide and (2-chloro-6-fluorophenyl)boronic acid. Flash column chromatography (DCM/MeOH 92:8). Obtained 0.056 g (48% of yield). ¹H NMR (500 MHz, DMSO-d₆) δ ppm 0.87 (t, J=7.32 Hz, 3H), 1.27 (m, 2H), 1.40 (m, 2H), 1.74-1.97 (m, 6H), 2.18-2.29 (m, 2H), 2.52-2.54 (m, 1H), 2.84-2.94 (m, 2H), 6.48 (d, J=9.00 Hz, 1H), 7.03 (d, J=8.69 Hz, 1H), 7.20-7.38 (m, 16H), 7.40-7.47 (m, 2H), 7.73 (s, 1H), 10.51 (br. s., 1H). HRMS (ESI) calcd for C42H41ClFN4O [M+H]⁺ 671.2948, found 671.2935.

### tert-Butyl[3-(4-{[5-(2-chloro-6-fluorophenyl)-1-trityl-1H-indazol-3-yl]carbamoyl}piperidin-1-yl)propyl]-carbamate [(XI), R1=2-chloro-6-fluorophenyl, R2=tert-butyl[3-(piperidin-1-yl)propyl]carbamate, R9=1,1′,1″-methanetriyltribenzene]

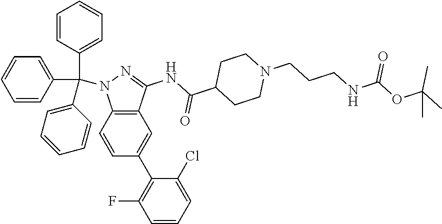 [SMILES: CC(C)(C)OC(=O)NCCCN1CCC(C(=O)Nc2nn(C(c3ccccc3)(c3ccccc3)c3ccccc3)c3ccc(-c4c(F)cccc4Cl)cc23)CC1]

Starting from tert-butyl (3-{4-[(5-bromo-1-trityl-1H-indazol-3-yl)carbamoyl]piperidin-1-yl}propyl)carbamate and (2-chloro-6-fluorophenyl)boronic acid. Flash column chromatography (DCM/MeOH 94:6). Obtained 0.061 g (54% of yield). ¹H NMR (500 MHz, DMSO-d₆) δ ppm 1.35 (s, 9H), 1.44-1.68 (m, 5H), 1.70-1.93 (m, 5H), 2.23 (m, 2H), 2.44 (m, 1H), 2.76-2.98 (m, 3H), 6.47 (d, J=9.15 Hz, 1H), 6.81 (br. s., 1H), 7.03 (d, J=8.54 Hz, 1H), 7.24-7.36 (m, 16H), 7.42-7.44 (m, 2H), 7.72 (s, 1H), 10.49 (br. s., 1H). HRMS (ESI) calcd for C46H48ClFN5O3 [M+H]⁺ 772.3424, found 772.3416.

### N-[5-(2-Chloro-6-fluorophenyl)-1-trityl-1H-indazol-3-yl]-1-(1-methoxypropan-2-yl)piperidine-4-carboxamide [(XI), R1=2-chloro-6-fluorophenyl, R2=1-(1-methoxypropan-2-yl)piperidine, R9=methanetriyltribenzene]

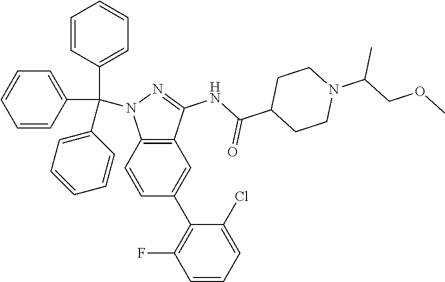 [SMILES: COCC(C)N1CCC(C(=O)Nc2nn(C(c3ccccc3)(c3ccccc3)c3ccccc3)c3ccc(-c4c(F)cccc4Cl)cc23)CC1]

Starting from N-(5-bromo-1-trityl-1H-indazol-3-yl)-1-(1-methoxypropan-2-yl)piperidine-4-carboxamide and (2-chloro-6-fluorophenyl)boronic acid. Flash column chromatography (DCM/MeOH 92:8). Obtained 0.032 g (52% of yield). LCMS (ESI) m/z 687 (M+H)⁺.

### N-[5-(2,6-Dichlorophenyl)-1-trityl-1H-indazol-3-yl]-1-methylpiperidine-4-carboxamide [(XI), R1=2,6-dichlorophenyl, R2=1-methylpiperidine, R9=1,1′,1″-methanetriyltribenzene]

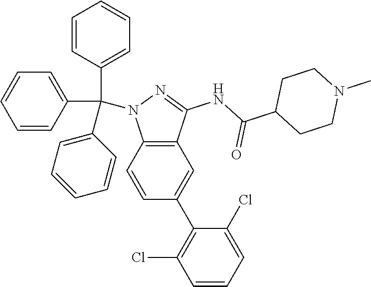 [SMILES: CN1CCC(C(=O)Nc2nn(C(c3ccccc3)(c3ccccc3)c3ccccc3)c3ccc(-c4c(Cl)cccc4Cl)cc23)CC1]

Starting from N-(5-bromo-1-trityl-1H-indazol-3-yl)-1-methylpiperidine-4-carboxamide and (2,6-dichlorophenyl)boronic acid. Flash column chromatography (DCM/MeOH 90:10). Obtained 0.106 g (64% of yield). ¹H NMR (500 MHz, DMSO-d₆) δ ppm 1.60-1.68 (m, 2H), 1.77 (d, J=10.68 Hz, 2H), 1.81-2.04 (m, 2H), 2.19 (br. s., 3H), 2.42 (m, 1H), 2.80-2.86 (m, 2H), 6.46 (d, J=9.00 Hz, 1H), 6.94 (d, J=8.54 Hz, 1H), 7.21-7.31 (m, 9H), 7.32-7.37 (m, 6H), 7.41 (t, J=8.24 Hz, 1H), 7.56 (d, J=8.08 Hz, 2H), 7.67 (s, 1H) 10.49 (br. s., 1H). HRMS (ESI) calcd for C39H35Cl2N4O [M+H]⁺ 645.2183, found 645.2186.

### 1-Butyl-N-[5-(2,6-dichlorophenyl)-1-trityl-1H-indazol-3-yl]piperidine-4-carboxamide [(XI), R1=2,6-dichlorophenyl, R2=1-butylpiperidine, R9=1,1′,1″-methanetriyltribenzene]

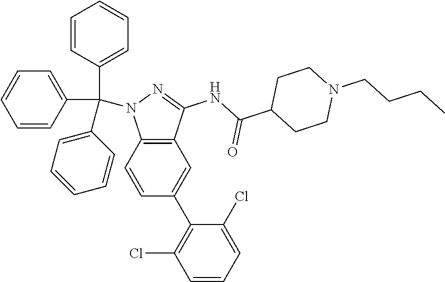 [SMILES: CCCCN1CCC(C(=O)Nc2nn(C(c3ccccc3)(c3ccccc3)c3ccccc3)c3ccc(-c4c(Cl)cccc4Cl)cc23)CC1]

Starting from N-(5-bromo-1-trityl-1H-indazol-3-yl)-1-butylpiperidine-4-carboxamide and (2,6-dichlorophenyl)boronic acid. Flash column chromatography (DCM/MeOH/33% aqueous ammonia 95:5:0.5). Obtained 0.144 g (79% of yield). ¹H NMR (500 MHz, DMSO-d₆) δ ppm 0.89 (t, J=7.32 Hz, 3H), 1.28 (m, 2H), 1.41 (m, 2H), 1.59-1.68 (m, 2H), 1.76-1.88 (m, 4H), 2.25 (m, 2H), 2.45 (m, 1H), 2.89 (d, J=9.00 Hz, 2H), 6.49 (d, J=9.00 Hz, 1H), 6.97 (d, J=8.69 Hz, 1H), 7.28-7.33 (m, 9H), 7.35-7.41 (m, 6H), 7.44 (t, J=8.39 Hz, 1H), 7.59 (d, J=8.08 Hz, 2H), 7.69 (s, 1H), 10.49 (s, 1H). HRMS (ESI) calcd for C42H41Cl2N4O [M+H]⁺ 687.2652, found 687.2680.

### N-[5-(2,6-Dichlorophenyl)-1-trityl-1H-indazol-3-yl]-1-(2-methylpropyl)piperidine-4-carboxamide [(XI), R1=2,6-dichlorophenyl, R2=1-(2-methylpropyl)piperidine, R9=1,1′,1″-methanetriyltribenzene]

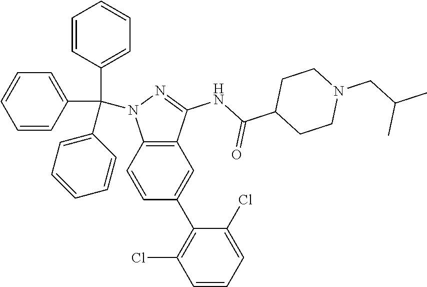 [SMILES: CC(C)CN1CCC(C(=O)Nc2nn(C(c3ccccc3)(c3ccccc3)c3ccccc3)c3ccc(-c4c(Cl)cccc4Cl)cc23)CC1]

Starting from 1 N-(5-bromo-1-trityl-1H-indazol-3-yl)-1-(2-methylpropyl)piperidine-4-carboxamide and (2,6-dichloro-phenyl)boronic acid. Flash column chromatography (DCM/MeOH 95:5). Obtained 0.105 g (70% of yield). LCMS (ESI) m/z 687 (M+H)⁺.

### N-[5-(2,6-Dichlorophenyl)-1-trityl-1H-indazol-3-yl]-1-(3-hydroxy-2,2-dimethylpropyl)piperidine-4-carboxamide [(XI), R1=2,6-dichlorophenyl, R2=1-(3-hydroxy-2,2-dimethylpropyl)piperidine, R9=methanetriyltribenzene]

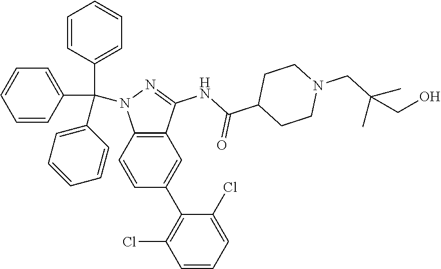 [SMILES: CC(C)(CO)CN1CCC(C(=O)Nc2nn(C(c3ccccc3)(c3ccccc3)c3ccccc3)c3ccc(-c4c(Cl)cccc4Cl)cc23)CC1]

Starting from N-(5-bromo-1-trityl-1H-indazol-3-yl)-1-(3-hydroxy-2,2-dimethylpropyl)piperidine-4-carboxamide and (2,6-dichlorophenyl)boronic acid. Flash column chromatography (DCM/MeOH 92:8). Obtained 0.077 g (62% of yield). ¹H NMR (401 MHz, DMSO-d₆) δ ppm 0.76 (s, 6H), 1.54-1.77 (m, 4H), 2.07-2.22 (m, 3H), 2.40 (m, 1H), 2.81 (d, J=9.64 Hz, 2H), 3.13 (s, 2H), 4.45 (m, 1H), 6.45 (d, J=8.79 Hz, 1H), 6.93 (d, J=9.03 Hz, 1H), 7.16-7.31 (m, 9H), 7.32-7.38 (m, 6H), 7.41 (t, J=8.67 Hz, 1H), 7.54 (d, J=8.18 Hz, 2H), 7.66 (s, 1H), 10.43 (br. s., 1H). HRMS (ESI) calcd for C43H43Cl2N4O2 [M+H]⁺ 717.2758, found 717.2746.

### N-[5-(6-Chloro-2-fluoro-3-methylphenyl)-1-trityl-1H-indazol-3-yl]-1-methylpiperidine-4-carboxamide [(XI), R1=6-chloro-2-fluoro-3-methylphenyl, R2=1-methylpiperidine, R9=1,1′,1″-methanetriyltribenzene]

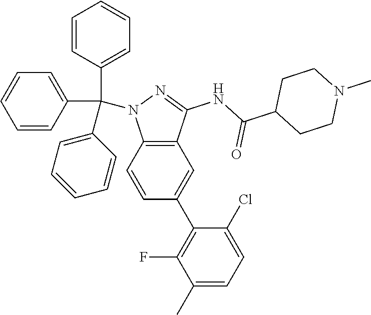 [SMILES: Cc1ccc(Cl)c(-c2ccc3c(c2)c(NC(=O)C2CCN(C)CC2)nn3C(c2ccccc2)(c2ccccc2)c2ccccc2)c1F]

Starting from N-(5-bromo-1-trityl-1H-indazol-3-yl)-1-methylpiperidine-4-carboxamide and (6-chloro-2-fluoro-3-methylphenyl)boronic acid. Flash column chromatography (DCM/MeOH/33% aqueous ammonia 94:6:0.5).

Obtained 0.060 g (64% of yield). 1H NMR (500 MHz, DMSO-d₆) δ ppm 1.60-1.65 (m, 2H), 1.73-1.75 (m, 2H), 1.81-1.84 (m, 2H), 2.13 (s, 3H), 2.23 (d, J=1.98 Hz, 3H), 2.40 (m, 1H), 2.78 (d, J=10.68 Hz, 2H), 6.46 (d, J=8.85 Hz, 1H), 7.00 (d, J=8.85 Hz, 1H), 7.21-7.28 (m, 6H), 7.28-7.32 (m, 5H), 7.32-7.39 (m, 6H), 7.72 (s, 1H), 10.47 (s, 1H). HRMS (ESI) calcd for C40H37ClFN4O [M+H]⁺ 643.2635, found 643.2640.

### N-[5-(2-Chloro-6-fluoro-3-methylphenyl)-1-trityl-1H-indazol-3-yl]-1-methylpiperidine-4-carboxamide [(XI), R1=2-chloro-6-fluoro-3-methylphenyl, R2=1-methylpiperidine, R9=1,1′,1″-methanetriyltribenzene]

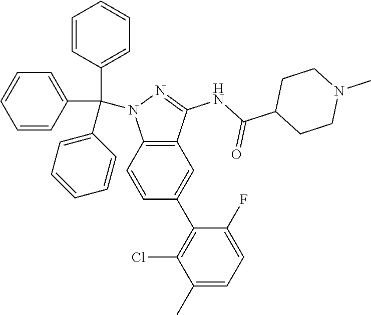 [SMILES: Cc1ccc(F)c(-c2ccc3c(c2)c(NC(=O)C2CCN(C)CC2)nn3C(c2ccccc2)(c2ccccc2)c2ccccc2)c1Cl]

Starting from N-(5-bromo-1-trityl-1H-indazol-3-yl)-1-methylpiperidine-4-carboxamide and (2-chloro-6-fluoro-3-methylphenyl)boronic acid. Flash column chromatography (DCM/MeOH/33% aqueous ammonia 90:10:0.5). Obtained 0.065 g (76% of yield). 1H NMR (500 MHz, DMSO-d₆) δ ppm 1.53-1.67 (m, 2H), 1.73-1.74 (m, 2H), 1.79-1.85 (m, 2H), 2.13 (s, 3H), 2.39 (m, 1H), 2.77 (d, J=11.13 Hz, 2H), 6.47 (d, J=9.00 Hz, 1H), 6.99 (d, J=9.30 Hz, 1H), 7.21 (t, J=8.69 Hz, 1H), 7.23-7.31 (m, 9H), 7.33-7.36 (m, 6H), 7.41 (m, 1H), 7.71 (s, 1H), 10.47 (s, 1H). HRMS (ESI) calcd for C40H37ClFN4O [M+H]⁺ 643.2635, found 643.2643.

### N-[5-(5-Chloro-2-fluoro-3-methylphenyl)-1-trityl-1H-indazol-3-yl]-1-methylpiperidine-4-carboxamide [(XI), R1=5-chloro-2-fluoro-3-methylphenyl, R2=1-methylpiperidine, R9=1,1′,1″-methanetriyltribenzene]

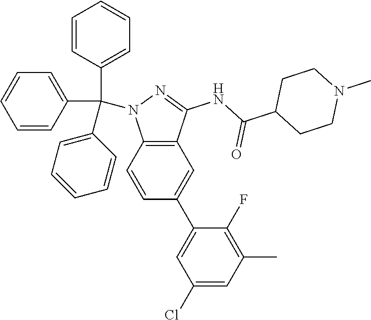 [SMILES: Cc1cc(Cl)cc(-c2ccc3c(c2)c(NC(=O)C2CCN(C)CC2)nn3C(c2ccccc2)(c2ccccc2)c2ccccc2)c1F]

Starting from N-(5-bromo-1-trityl-1H-indazol-3-yl)-1-methylpiperidine-4-carboxamide and (5-chloro-2-fluoro-3-methylphenyl)boronic acid. Flash column chromatography (DCM/MeOH/33% aqueous ammonia 92:8:0.5). Obtained 0.071 g (80% of yield). ¹H NMR (500 MHz, DMSO-d₆) δ ppm 1.60-1.72 (m, 2H), 1.76-1.79 (m, 2H), 1.82-1.87 (m, 2H), 2.14 (s, 3H), 2.26 (d, J=1.53 Hz, 3H), 2.41 (m, 1H), 2.79 (d, J=10.52 Hz, 2H), 6.44 (d, J=8.85 Hz, 1H), 7.17-7.25 (m, 6H), 7.26-7.31 (m, 5H), 7.32-7.40 (m, 7H), 7.88 (s, 1H), 10.47 (s, 1H). HRMS (ESI) calcd for C40H37ClFN4O [M+H]⁺ 643.2635, found 643.2640.

### N-{5-[2-Methoxy-5-(propan-2-yl)phenyl]-1-trityl-1H-indazol-3-yl}-1-methylpiperidine-4-carboxamide [(XI), R1=2-methoxy-5-(propan-2-yl)phenyl, R2=1-methylpiperidine, R9=1,1′,1″-methanetriyltribenzene]

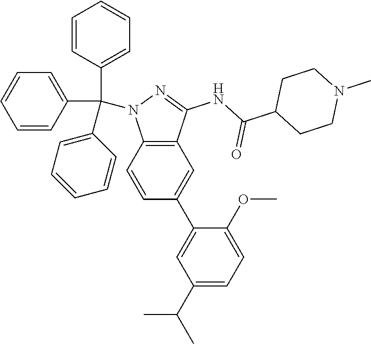 [SMILES: COc1ccc(C(C)C)cc1-c1ccc2c(c1)c(NC(=O)C1CCN(C)CC1)nn2C(c1ccccc1)(c1ccccc1)c1ccccc1]

Starting from N-(5-bromo-1-trityl-1H-indazol-3-yl)-1-methylpiperidine-4-carboxamide and [2-methoxy-5-(propan-2-yl)phenyl]boronic acid. Flash column chromatography (DCM/MeOH 90:10). Obtained 0.074 g (88% of yield). 1H NMR (500 MHz, DMSO-d₆) δ ppm 1.18 (d, J=6.86 Hz, 6H), 1.63-1.69 (m, 2H), 1.76-1.78 (m, 2H), 1.86-1.92 (m, 2H), 2.15 (br. s., 3H), 2.41 (m, 1H), 2.79 (d, J=9.00 Hz, 2H), 2.85 (m, 1H), 3.68 (s, 3H), 6.36 (d, J=8.85 Hz, 1H), 6.98 (d, J=8.54 Hz, 1H), 7.05 (s, 1H), 7.13 (d, J=9.30 Hz, 1H), 7.16 (dd, J=8.54, 2.29 Hz, 1H), 7.23-7.25 (m, 6H), 7.27-7.31 (m, 3H), 7.31-7.38 (m, 6H), 7.70 (s, 1H), 10.39 (s, 1H). HRMS (ESI) calcd for C43H45N4O2 [M+H]⁺649.3537, found 649.3548.

### 1-Methyl-N-{5-[3-(trifluoromethyl)phenyl]-1-trityl-1H-indazol-3-yl}piperidine-4-carboxamide [(XI), R1=3-(trifluoromethyl)phenyl, R2=1-methylpiperidine, R9=1,1′,1″-methanetriyltribenzene]

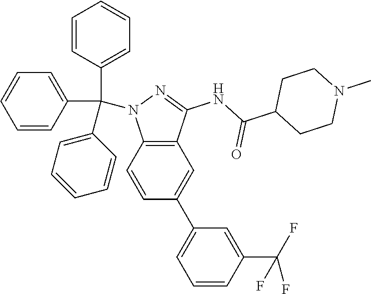 [SMILES: CN1CCC(C(=O)Nc2nn(C(c3ccccc3)(c3ccccc3)c3ccccc3)c3ccc(-c4cccc(C(F)(F)F)c4)cc23)CC1]

Starting from N-(5-bromo-1-trityl-1H-indazol-3-yl)-1-methylpiperidine-4-carboxamide and [3-(trifluoromethyl)phenyl]boronic acid. Flash column chromatography (DCM/MeOH 90:10). Obtained 0.033 g (40% of yield). LCMS (ESI) m/z 645 (M+H)⁺.

### N-{5-[2-Chloro-5-(hydroxymethyl)phenyl]-1-trityl-1H-indazol-3-yl}-1-methylpiperidine-4-carboxamide [(XI), R1=2-chloro-5-(hydroxymethyl)phenyl, R2=1-methylpiperidine, R9=1,1′,1″-methanetriyltribenzene]

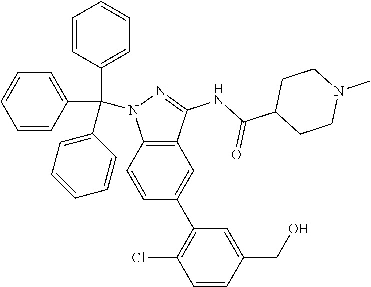 [SMILES: CN1CCC(C(=O)Nc2nn(C(c3ccccc3)(c3ccccc3)c3ccccc3)c3ccc(-c4cc(CO)ccc4Cl)cc23)CC1]

Starting from N-(5-bromo-1-trityl-1H-indazol-3-yl)-1-methylpiperidine-4-carboxamide and [2-chloro-5-(hydroxymethyl)phenyl]boronic acid. Flash column chromatography (DCM/MeOH/33% aqueous ammonia 92:8:0.5). Obtained 0.069 g (78% of yield). ¹H NMR (500 MHz, DMSO-d₆) δ ppm 1.56-1.70 (m, 2H), 1.76-1.78 (m, 2H), 1.82-1.89 (m, 2H), 2.14 (br. s., 3H), 2.41 (m, 1H), 2.79 (d, J=10.68 Hz, 2H), 4.50 (d, J=5.80 Hz, 2H), 5.28 (t, J=5.80 Hz, 1H), 6.44 (d, J=9.00 Hz, 1H), 7.10 (d, J=8.85 Hz, 1H), 7.24 (d, J=7.78 Hz, 6H), 7.26-7.32 (m, 5H), 7.32-7.38 (m, 6H), 7.47 (d, J=8.24 Hz, 1H), 7.76 (s, 1H), 10.46 (s, 1H). HRMS (ESI) calcd for C40H38ClN4O2 [M+H]⁺ 641.2678, found 641.2698.

### N-[5-(2-Fluorophenyl)-1-trityl-1H-indazol-3-yl]-1-methylpiperidine-4-carboxamide [(XI), R1=2-fluorophenyl, R2=1-methylpiperidine, R9=1,1′,1″-methanetriyltribenzene]

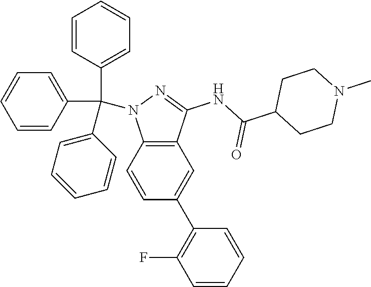 [SMILES: CN1CCC(C(=O)Nc2nn(C(c3ccccc3)(c3ccccc3)c3ccccc3)c3ccc(-c4ccccc4F)cc23)CC1]

Starting from N-(5-bromo-1-trityl-1H-indazol-3-yl)-1-methylpiperidine-4-carboxamide and (2-fluorophenyl)boronic acid. Flash column chromatography (DCM/MeOH/33% aqueous ammonia 90:10:0.5). Obtained 0.071 g (87% of yield). ¹H NMR (500 MHz, DMSO-d₆) δ ppm 1.58-1.72 (m, 2H), 1.77-1.86 (m, 4H), 2.14 (s, 3H), 2.41 (m, 1H), 2.79 (d, J=10.52 Hz, 2H), 6.45 (d, J=8.85 Hz, 1H), 7.17-7.45 (m, 20H), 7.86 (s, 1H), 10.47 (s, 1H). HRMS (ESI) calcd for C39H36FN4O [M+H]⁺ 595.2868, found 595.2884.

### N-[5-(2-Cyanophenyl)-1-trityl-1H-indazol-3-yl]-1-methylpiperidine-4-carboxamide [(XI), R1=2-cyanophenyl, R2=1-methylpiperidine, R9=1,1′,1″-methanetriyltribenzene]

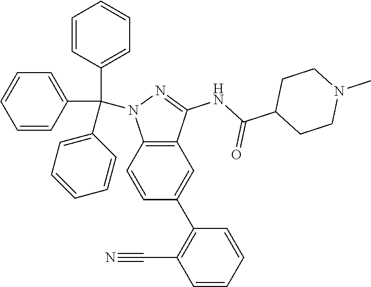 [SMILES: CN1CCC(C(=O)Nc2nn(C(c3ccccc3)(c3ccccc3)c3ccccc3)c3ccc(-c4ccccc4C#N)cc23)CC1]

Starting from N-(5-bromo-1-trityl-1H-indazol-3-yl)-1-methylpiperidine-4-carboxamide and (2-cyanophenyl)boronic acid. Flash column chromatography (DCM/MeOH/33% aqueous ammonia 90:10:0.5). Obtained 0.059 g (71% of yield). ¹H NMR (500 MHz, DMSO-d₆) δ ppm 1.60-1.71 (m, 2H), 1.75-1.78 (m, 2H), 1.82-1.87 (m, 2H), 2.14 (s, 3H), 2.42 (m, 1H), 2.79 (d, J=10.37 Hz, 2H), 6.48 (d, J=8.85 Hz, 1H), 7.22-7.27 (m, 6H), 7.28-7.32 (m, 4H), 7.33-7.38 (m, 6H), 7.54 (td, J=7.63, 1.07 Hz, 1H), 7.58 (d, J=7.93 Hz, 1H), 7.75 (m, 1H), 7.91 (d, J=7.93 Hz, 1H), 7.95 (s, 1H), 10.53 (s, 1H). HRMS (ESI) calcd for C40H36N5O [M+H]⁺ 602.2915, found 602.2935.

### N-[5-(2,5-Dichlorophenyl)-1-trityl-1H-indazol-3-yl]-1-methylpiperidine-4-carboxamide [(XI), R1=2,5-dichlorophenyl, R2=1-methylpiperidine, R9=1,1′,1″-methanetriyltribenzene]

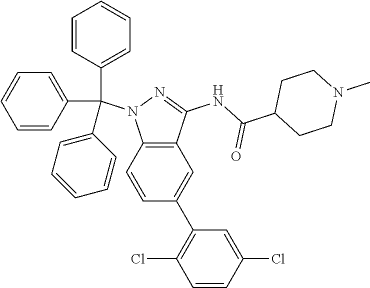 [SMILES: CN1CCC(C(=O)Nc2nn(C(c3ccccc3)(c3ccccc3)c3ccccc3)c3ccc(-c4cc(Cl)ccc4Cl)cc23)CC1]

Starting from N-(5-bromo-1-trityl-1H-indazol-3-yl)-1-methylpiperidine-4-carboxamide and (2,5-dichlorophenyl)boronic acid. Flash column chromatography (DCM/MeOH/33% aqueous ammonia 90:10:0.5). Obtained 0.074 g (83% of yield). ¹H NMR (500 MHz, DMSO-d₆) δ ppm 1.56-1.70 (m, 2H), 1.73-1.77 (m, 2H), 1.81-1.86 (m, 2H), 2.13 (s, 3H), 2.40 (m, 1H), 2.78 (d, J=10.52 Hz, 2H), 6.45 (d, J=9.00 Hz, 1H), 7.14 (d, J=8.54 Hz, 1H), 7.24 (d, J=7.63 Hz, 6H), 7.26-7.31 (m, 3H), 7.32-7.39 (m, 6H), 7.42-7.47 (m, 2H), 7.57 (d, J=8.69 Hz, 1H), 7.81 (s, 1H), 10.48 (s, 1H). HRMS (ESI) calcd for C39H35Cl2N4O [M+H]⁺ 645.2183, found 645.2198.

### N-[5-(3-Carbamoylphenyl)-1-trityl-1H-indazol-3-yl]-1-methylpiperidine-4-carboxamide [(XI), R1=3-carbamoylphenyl, R2=1-methylpiperidine, R9=1,1′,1″-methanetriyltribenzene]

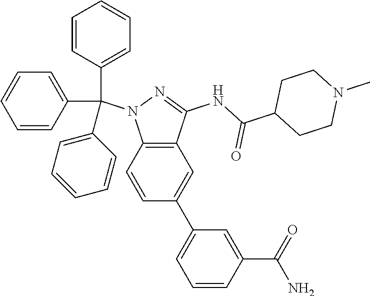 [SMILES: CN1CCC(C(=O)Nc2nn(C(c3ccccc3)(c3ccccc3)c3ccccc3)c3ccc(-c4cccc(C(N)=O)c4)cc23)CC1]

Starting from N-(5-bromo-1-trityl-1H-indazol-3-yl)-1-methylpiperidine-4-carboxamide and (3-carbamoylphenyl)boronic acid. Flash column chromatography (DCM/MeOH/33% aqueous ammonia 90:10:1). Obtained 0.077 g (91% of yield). ¹H NMR (500 MHz, DMSO-d₆) δ ppm 1.62-1.74 (m, 2H), 1.76-1.92 (m, 4H), 2.15 (br. s., 3H), 2.43 (m, 1H), 2.81 (d, J=9.61 Hz, 2H), 6.44 (d, J=9.00 Hz, 1H), 7.23 (d, J=7.63 Hz, 6H), 7.27-7.31 (m, 4H), 7.32-7.37 (m, 3H), 7.39-7.43 (m, 2H), 7.49 (t, J=7.78 Hz, 1H), 7.70 (d, J=7.02 Hz, 1H), 7.81 (d, J=7.78 Hz, 1H), 7.96 (s, 1H), 8.03-8.06 (m, 2H), 10.45 (s, 1H). HRMS (ESI) calcd for C40H38N5O2 [M+H]⁺ 620.3020, found 620.3038.

### N-{5-[4-(Hydroxymethyl)phenyl]-1-trityl-1H-indazol-3-yl}-1-methylpiperidine-4-carboxamide [(XI), R1=4-(hydroxymethyl)phenyl, R2=1-methylpiperidine, R9=1,1′,1″-methanetriyltribenzene]

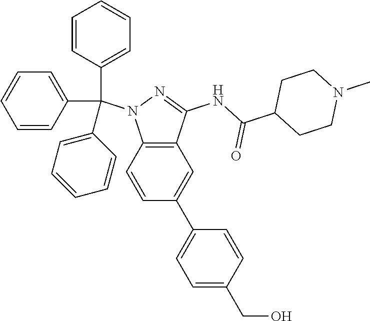 [SMILES: CN1CCC(C(=O)Nc2nn(C(c3ccccc3)(c3ccccc3)c3ccccc3)c3ccc(-c4ccc(CO)cc4)cc23)CC1]

Starting from N-(5-bromo-1-trityl-1H-indazol-3-yl)-1-methylpiperidine-4-carboxamide and [4-(hydroxymethyl)phenyl]boronic acid. Flash column chromatography (DCM/MeOH/33% aqueous ammonia 90:10:0.5). Obtained 0.073 g (87% of yield). ¹H NMR (500 MHz, DMSO-d₆) δ ppm 1.60-1.75 (m, 2H), 1.76-1.94 (m, 4H), 2.15 (s, 3H), 2.42 (m, 1H), 2.80 (d, J=10.83 Hz, 2H), 4.51 (d, J=5.80 Hz, 2H), 5.19 (t, J=5.72 Hz, 1H), 6.42 (d, J=8.85 Hz, 1H), 7.22 (d, J=7.47 Hz, 6H), 7.27-7.31 (m, 3H), 7.32-7.38 (m, 10H), 7.50 (d, J=7.78 Hz, 1H), 7.90 (s, 1H), 10.43 (s, 1H). HRMS (ESI) calcd for C40H39N4O2 [M+H]⁺ 607.3068, found 607.3079.

### N-{5-[3-(Hydroxymethyl)phenyl]-1-trityl-1H-indazol-3-yl}-1-methylpiperidine-4-carboxamide [(XI), R1=3-(hydroxymethyl)phenyl, R2=1-methylpiperidine, R9=1,1′,1″-methanetriyltribenzene]

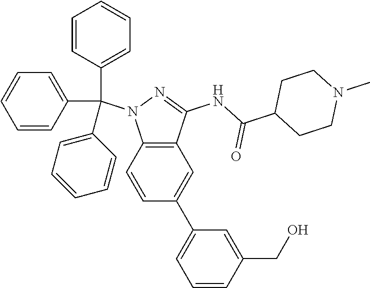 [SMILES: CN1CCC(C(=O)Nc2nn(C(c3ccccc3)(c3ccccc3)c3ccccc3)c3ccc(-c4cccc(CO)c4)cc23)CC1]

Starting from N-(5-bromo-1-trityl-1H-indazol-3-yl)-1-methylpiperidine-4-carboxamide and [3-(hydroxymethyl)phenyl]boronic acid. Flash column chromatography (DCM/MeOH/33% aqueous ammonia 90:10:0.5). Obtained 0.068 g (81% of yield). ¹H NMR (500 MHz, DMSO-d₆) δ ppm 1.62-1.74 (m, 2H), 1.79-1.91 (m, 4H), 2.16 (br. s., 2H), 2.43 (m, 1H), 2.81 (d, J=9.46 Hz, 2H), 4.53 (d, J=5.64 Hz, 2H), 5.22 (t, J=5.72 Hz, 1H), 6.43 (d, J=9.00 Hz, 1H), 7.18-7.43 (m, 19H), 7.48 (m, 1H), 7.90 (s, 1H), 10.44 (s, 1H). HRMS (ESI) calcd for C40H39N4O2 [M+H]⁺ 607.3068, found 607.3085.

### 1-Methyl-N-[5-(pyridin-3-yl)-1-trityl-1H-indazol-3-yl]piperidine-4-carboxamide [(XI), R1=pyridin-3-yl, R2=1-methylpiperidine, R9=1,1′,1″-methanetriyltribenzene]

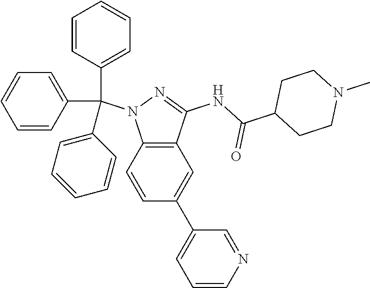 [SMILES: CN1CCC(C(=O)Nc2nn(C(c3ccccc3)(c3ccccc3)c3ccccc3)c3ccc(-c4cccnc4)cc23)CC1]

Starting from N-(5-bromo-1-trityl-1H-indazol-3-yl)-1-methylpiperidine-4-carboxamide and pyridin-3-ylboronic acid. Flash column chromatography (DCM/MeOH 90:10). Obtained 0.055 g (73% of yield). ¹H NMR (500 MHz, DMSO-d₆) δ ppm 1.76-1.86 (m, 2H), 1.92-1.99 (m, 2H), 2.20 (br. s., 2H), 2.45 (m, 1H), 3.13 (m, 2H), 6.48 (d, J=8.85 Hz, 1H), 7.22 (d, J=7.63 Hz, 6H), 7.26-7.32 (m, 3H), 7.32-7.39 (m, 6H), 7.42 (d, J=8.85 Hz, 1H), 7.45 (dd, J=7.93, 4.88 Hz, 1H), 7.97 (d, J=7.78 Hz, 1H), 7.99 (s, 1H), 8.53 (dd, J=4.73, 1.37 Hz, 1H), 8.79 (s, 1H), 10.61 (br. s., 1H). HRMS (ESI) calcd for C38H36N5O [M+H]⁺ 578.2915, found 578.2916.

### N-[5-(2-Chlorophenyl)-1-trityl-1H-indazol-3-yl]-1-methylpiperidine-4-carboxamide [(XI), R1=2-chlorophenyl, R2=1-methylpiperidine, R9=1,1′,1″-methanetriyltribenzene]

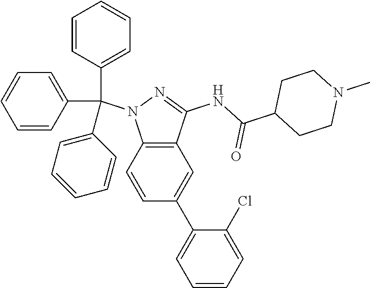 [SMILES: CN1CCC(C(=O)Nc2nn(C(c3ccccc3)(c3ccccc3)c3ccccc3)c3ccc(-c4ccccc4Cl)cc23)CC1]

Starting from N-(5-bromo-1-trityl-1H-indazol-3-yl)-1-methylpiperidine-4-carboxamide and (2-chlorophenyl)boronic acid. Flash column chromatography (DCM/MeOH 90:10). Obtained 0.041 g (51% of yield). LCMS (ESI) m/z 612 (M+H)⁺.

### N-[5-(2-Chloro-5-methylphenyl)-1-trityl-1H-indazol-3-yl]-1-methylpiperidine-4-carboxamide [(XI), R1=2-chloro-5-methylphenyl, R2=1-methylpiperidine, R9=1,1′,1″-methanetriyltribenzene]

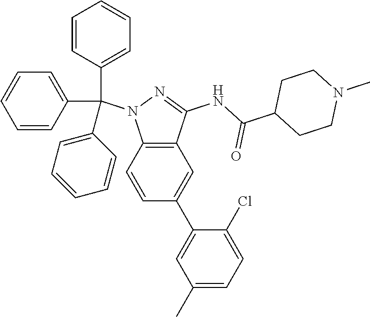 [SMILES: Cc1ccc(Cl)c(-c2ccc3c(c2)c(NC(=O)C2CCN(C)CC2)nn3C(c2ccccc2)(c2ccccc2)c2ccccc2)c1]

Starting from N-(5-bromo-1-trityl-1H-indazol-3-yl)-1-methylpiperidine-4-carboxamide and (2-chloro-5-methylpheny)lboronic acid. Flash column chromatography (DCM/MeOH/33% aqueous ammonia 90:10:0.5). Obtained 0.057 g (66% of yield). ¹H NMR (500 MHz, DMSO-d₆) δ ppm 1.58-1.70 (m, 2H), 1.74-1.77 (m, 2H), 1.81-1.85 (m, 2H), 2.13 (s, 3H), 2.40 (m, 1H), 2.78 (d, J=10.98 Hz, 2H), 6.43 (d, J=8.85 Hz, 1H), 7.09 (d, J=8.85 Hz, 1H), 7.17-7.18 (m, 2H), 7.24 (d, J=7.63 Hz, 6H), 7.27-7.32 (m, 3H), 7.33-7.37 (m, 6H), 7.38 (d, J=8.69 Hz, 1H), 7.75 (s, 1H), 10.45 (s, 1H). HRMS (ESI) calcd for C40H38ClN4O [M+H]⁺ 625.2729, found 625.2744.

### N-[5-(4-Fluorophenyl)-1-trityl-1H-indazol-3-yl]-1-methylpiperidine-4-carboxamide [(XI), R1=4-fluorophenyl, R2=1-methylpiperidine, R9=1,1′,1″-methanetriyltribenzene]

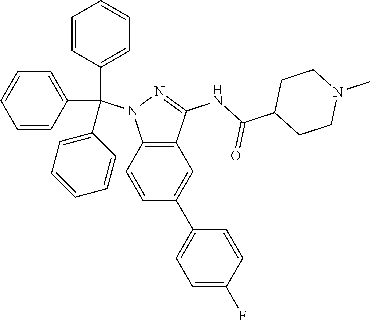 [SMILES: CN1CCC(C(=O)Nc2nn(C(c3ccccc3)(c3ccccc3)c3ccccc3)c3ccc(-c4ccc(F)cc4)cc23)CC1]

Starting from N-(5-bromo-1-trityl-1H-indazol-3-yl)-1-methylpiperidine-4-carboxamide and (4-fluorophenyl)boronic acid. Flash column chromatography (DCM/MeOH/33% aqueous ammonia 92:8:0.5). Obtained 0.078 g (95% of yield). ¹H NMR (500 MHz, DMSO-d₆) δ ppm 1.65-1.71 (m, 2H), 1.75-1.93 (m, 4H), 2.15 (s, 3H), 2.42 (m, 1H), 2.81 (d, J=10.83 Hz, 2H), 6.43 (d, J=8.85 Hz, 1H), 7.19-7.25 (m, 6H), 7.25-7.31 (m, 6H), 7.32-7.37 (m, 6H), 7.59 (m, 2H), 7.89 (s, 1H), 10.44 (s, 1H). HRMS (ESI) calcd for C39H36FN4O [M+H]⁺ 595.2868, found 595.2878.

### N-[5-(2-Ethylphenyl)-1-trityl-1H-indazol-3-yl]-1-methylpiperidine-4-carboxamide [(XI), R1=2-ethylphenyl, R2=1-methylpiperidine, R9=1,1′,1″-methanetriyltribenzene]

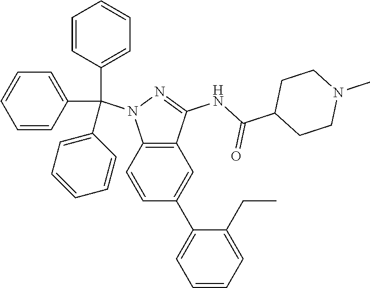 [SMILES: CCc1ccccc1-c1ccc2c(c1)c(NC(=O)C1CCN(C)CC1)nn2C(c1ccccc1)(c1ccccc1)c1ccccc1]

Starting from N-(5-bromo-1-trityl-1H-indazol-3-yl)-1-methylpiperidine-4-carboxamide and (2-ethylphenyl)boronic acid. Flash column chromatography (DCM/MeOH/33% aqueous ammonia 90:10:0.5). Obtained 0.067 g (81% of yield). ¹H NMR (500 MHz, DMSO-d₆) δ ppm 1.01 (t, J=7.47 Hz, 3H), 1.58-1.68 (m, 2H), 1.73-1.76 (m, 2H), 1.80-1.84 (m, 2H), 2.12 (s, 3H), 2.39 (m, 1H), 2.47 (m, 2H) 2.77 (d, J=10.83 Hz, 2H), 6.43 (d, J=9.00 Hz, 1H), 7.00 (d, J=8.69 Hz, 1H), 7.10 (d, J=7.32 Hz, 1H), 7.20 (td, J=7.09, 1.98 Hz, 1H), 7.23-7.31 (m, 11H), 7.32-7.38 (m, 6H), 7.60 (s, 1H), 10.43 (s, 1H). HRMS (ESI) calcd for C41H41N4O [M+H]⁺ 605.3275, found 605.3289.

### N-[5-(3-Cyanophenyl)-1-trityl-1H-indazol-3-yl]-1-methylpiperidine-4-carboxamide [(XI), R1=3-cyanophenyl, R2=1-methylpiperidine, R9=1,1′,1″-methanetriyltribenzene]

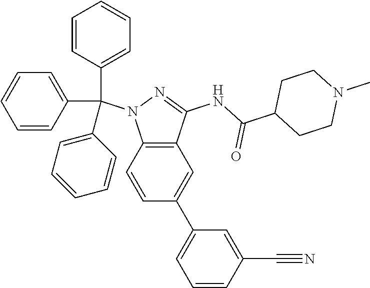 [SMILES: CN1CCC(C(=O)Nc2nn(C(c3ccccc3)(c3ccccc3)c3ccccc3)c3ccc(-c4cccc(C#N)c4)cc23)CC1]

Starting from N-(5-bromo-1-trityl-1H-indazol-3-yl)-1-methylpiperidine-4-carboxamide and (3-cyanophenyl)boronic acid. Flash column chromatography (DCM/MeOH/33% aqueous ammonia 90:10:0.5). Obtained 0.070 g (85% of yield). 1H NMR (500 MHz, DMSO-d₆) δ ppm 1.62-1.74 (m, 2H), 1.77-1.92 (m, 4H), 2.15 (s, 3H), 2.42 (m, 1H), 2.81 (d, J=10.52 Hz, 2H), 6.45 (d, J=8.85 Hz, 1H), 7.22 (d, J=7.63 Hz, 6H), 7.27-7.31 (m, 3H), 7.33-7.38 (m, 6H), 7.42 (d, J=8.54 Hz, 1H), 7.64 (t, J=7.78 Hz, 1H), 7.77-7.80 (m, 1H), 7.91 (d, J=7.93 Hz, 1H), 8.00-8.02 (m, 2H), 10.47 (s, 1H). HRMS (ESI) calcd for C40H36N5O [M+H]⁺ 602.2915, found 602.2935.

### N-[5-(4-Aminophenyl)-1-trityl-1H-indazol-3-yl]-1-methylpiperidine-4-carboxamide [(XI), R1=4-aminophenyl, R2=1-methylpiperidine, R9=1,1′,1″-methanetriyltribenzene]

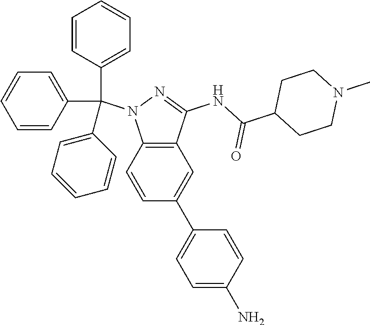 [SMILES: CN1CCC(C(=O)Nc2nn(C(c3ccccc3)(c3ccccc3)c3ccccc3)c3ccc(-c4ccc(N)cc4)cc23)CC1]

Starting from N-(5-bromo-1-trityl-1H-indazol-3-yl)-1-methylpiperidine-4-carboxamide and (4-aminophenyl)boronic acid. Flash column chromatography (DCM/MeOH 90:10). Obtained 0.059 g (77% of yield). ¹H NMR (500 MHz, DMSO-d₆) δ ppm 1.67-1.76 (m, 4H), 1.81-1.86 (m, 2H), 2.26 (br. s., 3H), 2.46 (m, 1H), 2.91 (m, 2H), 5.15 (s, 2H), 6.35 (d, J=9.00 Hz, 1H), 6.60 (d, J=8.54 Hz, 2H), 7.21-7.25 (m, 8H), 7.26-7.30 (m, 4H), 7.31-7.36 (m, 6H), 7.72 (s, 1H), 10.39 (br. s., 1H). HRMS (ESI) calcd for C39H38N5O [M+H]⁺ 592.3071, found 592.3098.

### N-[5-(2-Aminophenyl)-1-trityl-1H-indazol-3-yl]-1-methylpiperidine-4-carboxamide [(XI), R1=2-aminophenyl, R2=1-methylpiperidine, R9=1,1′,1″-methanetriyltribenzene]

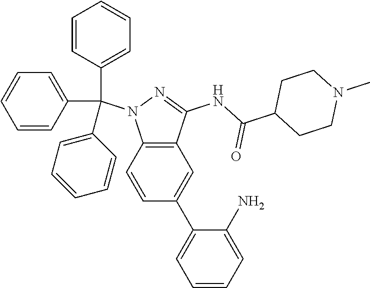 [SMILES: CN1CCC(C(=O)Nc2nn(C(c3ccccc3)(c3ccccc3)c3ccccc3)c3ccc(-c4ccccc4N)cc23)CC1]

Starting from N-(5-bromo-1-trityl-1H-indazol-3-yl)-1-methylpiperidine-4-carboxamide and (2-aminophenyl)boronic acid. Flash column chromatography (DCM/MeOH 90:10). Obtained 0.044 g (57% of yield). 1H NMR (500 MHz, DMSO-d₆) δ ppm 1.63-1.68 (m, 2H), 1.74-1.77 (m, 2H), 1.79-1.90 (m, 2H), 2.13 (s, 3H), 2.39 (m, 1H), 2.78 (m, J=10.83 Hz, 2H), 4.68 (s, 2H), 6.42 (d, J=8.85 Hz, 1H), 6.60 (td, J=7.40, 1.07 Hz, 1H), 6.72 (d, J=8.08 Hz, 1H), 6.91 (d, J=7.02 Hz, 1H), 7.01 (m, 1H), 7.06 (d, J=8.54 Hz, 1H), 7.24 (d, J=7.63 Hz, 6H), 7.27-7.32 (m, 3H), 7.32-7.39 (m, 6H), 7.70 (s, 1H), 10.41 (s, 1H). HRMS (ESI) calcd for C39H38N5O [M+H]⁺ 592.3071, found 592.3093.

### 1-Methyl-N-[5-(4-sulfamoylphenyl)-1-trityl-1H-indazol-3-yl]piperidine-4-carboxamide [(XI), R1=4-sulfamoylphenyl, R2=1-methylpiperidine, R9=1,1′,1″-methanetriyltribenzene]

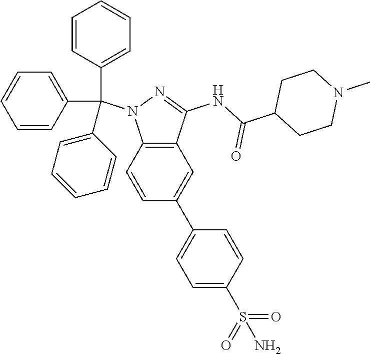 [SMILES: CN1CCC(C(=O)Nc2nn(C(c3ccccc3)(c3ccccc3)c3ccccc3)c3ccc(-c4ccc(S(N)(=O)=O)cc4)cc23)CC1]

Starting from N-(5-bromo-1-trityl-1H-indazol-3-yl)-1-methylpiperidine-4-carboxamide and (4-sulfamoylphenyl)boronic acid. Flash column chromatography (DCM/MeOH/33% aqueous ammonia 90:10:0.5). Obtained 0.065 g (72% of yield). ¹H NMR (500 MHz, DMSO-d₆) δ ppm 1.65-1.72 (m, 2H), 1.77-1.93 (m, 4H), 2.16 (br. s., 3H), 2.43 (m, 1H), 2.81 (m, J=9.30 Hz, 2H), 6.46 (d, J=9.00 Hz, 1H), 7.22 (d, J=7.47 Hz, 6H), 7.26-7.31 (m, 3H), 7.32-7.38 (m, 8H), 7.42 (d, J=8.85 Hz, 1H), 7.75 (d, J=8.08 Hz, 2H), 7.85 (d, J=8.54 Hz, 2H), 8.01 (s, 1H), 10.48 (s, 1H). HRMS (ESI) calcd for C39H38N5O3S [M+H]⁺ 656.2690, found 656.2707.

### 1-Methyl-N-(5-phenyl-1-trityl-1H-indazol-3-yl)piperidine-4-carboxamide [(XI), R1=phenyl, R2=1-methylpiperidine, R9=1,1′,1″-methanetriyltribenzene]

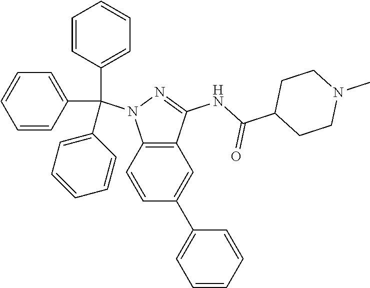 [SMILES: CN1CCC(C(=O)Nc2nn(C(c3ccccc3)(c3ccccc3)c3ccccc3)c3ccc(-c4ccccc4)cc23)CC1]

Starting from N-(5-bromo-1-trityl-1H-indazol-3-yl)-1-methylpiperidine-4-carboxamide and phenyllboronic acid. Flash column chromatography (DCM/MeOH 90:10). Obtained 0.062 g (83% of yield). ¹H NMR (500 MHz, DMSO-d₆) δ ppm 1.62-1.74 (m, 2H), 1.79-1.87 (m, 4H), 2.15 (s, 3H), 2.42 (m, 1H), 2.80 (d, J=10.52 Hz, 2H), 6.44 (d, J=9.00 Hz, 1H), 7.23 (d, J=7.63 Hz, 6H), 7.27-7.36.38 (m, 10H), 7.41-7.44 (m, 3H), 7.55 (d, J=7.47 Hz, 2H), 7.92 (s, 1H), 10.44 (s, 1H). HRMS (ESI) calcd for C39H37N4O [M+H]⁺ 577.2962, found 577.2974.

### N-{5-[2-Chloro-5-(trifluoromethyl)phenyl]-1-trityl-1H-indazol-3-yl}-1-methylpiperidine-4-carboxamide [(XI), R1=2-chloro-5-(trifluoromethyl)phenyl, R2=1-methylpiperidine, R9=1,1′,1″-methanetriyltribenzene]

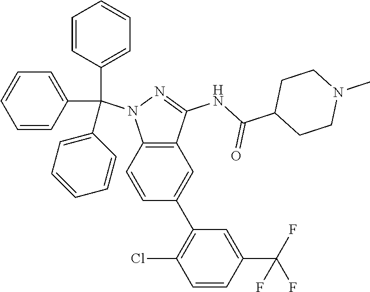 [SMILES: CN1CCC(C(=O)Nc2nn(C(c3ccccc3)(c3ccccc3)c3ccccc3)c3ccc(-c4cc(C(F)(F)F)ccc4Cl)cc23)CC1]

Starting from N-(5-bromo-1-trityl-1H-indazol-3-yl)-1-methylpiperidine-4-carboxamide and [2-chloro-5-(trifluoromethyl)phenyl]boronic acid. Flash column chromatography (DCM/MeOH 90:10). Obtained 0.055 g (62% of yield). ¹H NMR (500 MHz, DMSO-d₆) δ ppm 1.62-1.66 (m, 2H), 1.74-1.77 (m, 2H), 1.81-1.85 (m, 2H), 2.13 (s, 3H), 2.40 (m, 1H), 2.77 (d, J=10.52 Hz, 2H), 6.46 (d, J=8.69 Hz, 1H), 7.17 (d, J=9.00 Hz, 1H), 7.24 (d, J=7.63 Hz, 6H), 7.27-7.32 (m, 3H), 7.32-7.38 (m, 6H), 7.68 (s, 1H), 7.73 (dd, J=8.39, 2.14 Hz, 1H), 7.79 (d, J=8.39 Hz, 1H), 7.84 (s, 1H), 10.49 (s, 1H). HRMS (ESI) calcd for C40H35ClF3N4O [M+H]⁺ 679.2446, found 679.2467.

### N-[5-(2-Fluoropyridin-3-yl)-1-trityl-1H-indazol-3-yl]-1-methylpiperidine-4-carboxamide [(XI), R1=2-fluoropyridin-3-yl, R2=1-methylpiperidine, R9=1,1′,1″-methanetriyltribenzene]

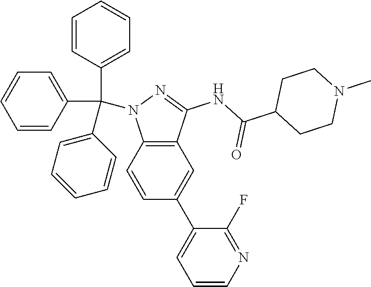 [SMILES: CN1CCC(C(=O)Nc2nn(C(c3ccccc3)(c3ccccc3)c3ccccc3)c3ccc(-c4cccnc4F)cc23)CC1]

Starting from N-(5-bromo-1-trityl-1H-indazol-3-yl)-1-methylpiperidine-4-carboxamide and (2-fluoropyridin-3-yl)boronic acid. Flash column chromatography (DCM/MeOH 90:10). Obtained 0.068 g (87% of yield). ¹H NMR (500 MHz, DMSO-d₆) δ ppm 1.57-1.72 (m, 2H), 1.77-1.80 (m, 2H), 1.82-1.96 (m, 2H), 2.15 (br. s., 3H), 2.42 (m, 1H), 2.80 (d, J=10.07 Hz, 2H), 6.47 (d, J=8.85 Hz, 1H), 7.23 (d, J=7.63 Hz, 6H), 7.26-7.31 (m, 4H), 7.31-7.40 (m, 6H), 7.44 (ddd, J=7.17, 5.11, 1.75 Hz, 1H), 7.94 (s, 1H), 8.04 (t, J=9.30 Hz, 1H), 8.19 (d, J=4.88 Hz, 1H), 10.50 (s, 1H). HRMS (ESI) calcd for C38H35FN5O [M+H]⁺ 596.2820, found 596.2834.

### N-[5-(2-Methoxyphenyl)-1-trityl-1H-indazol-3-yl]-1-methylpiperidine-4-carboxamide [(XI), R1=2-methoxyphenyl, R2=1-methylpiperidine, R9=1,1′,1″-methanetriyltribenzene]

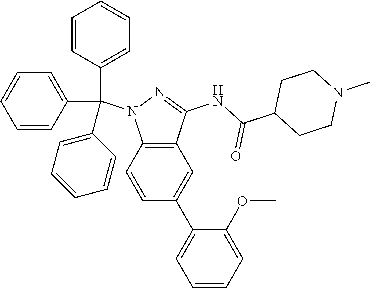 [SMILES: COc1ccccc1-c1ccc2c(c1)c(NC(=O)C1CCN(C)CC1)nn2C(c1ccccc1)(c1ccccc1)c1ccccc1]

Starting from N-(5-bromo-1-trityl-1H-indazol-3-yl)-1-methylpiperidine-4-carboxamide and (2-methoxyphenyl)boronic acid. Flash column chromatography (DCM/MeOH 90:10). Obtained 0.056 g (71% of yield). ¹H NMR (500 MHz, DMSO-d₆) δ ppm 1.69-1.77 (m, 2H), 1.86-1.89 (m, 2H), 2.39 (m, 1H), 3.04 (m, 2H), 3.71 (s, 3H), 6.37 (d, J=9.00 Hz, 1H), 6.99 (td, J=7.43, 0.69 Hz, 1H), 7.08 (d, J=8.24 Hz, 1H), 7.16 (d, J=9.00 Hz, 1H), 7.20-7.26 (m, 7H), 7.26-7.32 (m, 3H), 7.32-7.41 (m, 7H), 7.73 (s, 1H), 10.49 (br. s., 1H). HRMS (ESI) calcd for C40H39N4O2 [M+H]⁺ 607.3068, found 607.3087.

### N-[5-(2,3-Dichlorophenyl)-1-trityl-1H-indazol-3-yl]-1-methylpiperidine-4-carboxamide [(XI), R1=2,3-dichlorophenyl, R2=1-methylpiperidine, R9=1,1′,1″-methanetriyltribenzene]

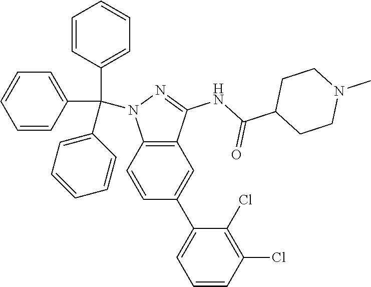 [SMILES: CN1CCC(C(=O)Nc2nn(C(c3ccccc3)(c3ccccc3)c3ccccc3)c3ccc(-c4cccc(Cl)c4Cl)cc23)CC1]

Starting from N-(5-bromo-1-trityl-1H-indazol-3-yl)-1-methylpiperidine-4-carboxamide and (2,3-dichlorophenyl)boronic acid. Flash column chromatography (DCM/MeOH 90:10). Obtained 0.043 g (56% of yield). LCMS (ESI) m/z 646 (M+H)⁺.

### N-[5-(2-Chloro-6-methoxyphenyl)-1-trityl-1H-indazol-3-yl]-1-methylpiperidine-4-carboxamide [(XI), R1=2-chloro-6-methoxyphenyl, R2=1-methylpiperidine, R9=1,1′,1″-methanetriyltribenzene]

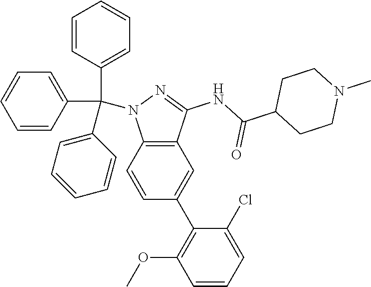 [SMILES: COc1cccc(Cl)c1-c1ccc2c(c1)c(NC(=O)C1CCN(C)CC1)nn2C(c1ccccc1)(c1ccccc1)c1ccccc1]

Starting from N-(5-bromo-1-trityl-1H-indazol-3-yl)-1-methylpiperidine-4-carboxamide and (2-chloro-6-methoxyphenyl)boronic acid. Flash column chromatography (DCM/MeOH 90:10). Obtained 0.048 g (57% of yield). LCMS (ESI) m/z 641 (M+H)⁺.

### N-[5-(2,3-Difluorophenyl)-1-trityl-1H-indazol-3-yl]-1-methylpiperidine-4-carboxamide [(XI), R1=2,3-difluorophenyl, R2=1-methylpiperidine, R9=1,1′,1″-methanetriyltribenzene]

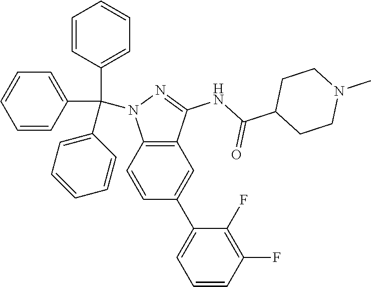 [SMILES: CN1CCC(C(=O)Nc2nn(C(c3ccccc3)(c3ccccc3)c3ccccc3)c3ccc(-c4cccc(F)c4F)cc23)CC1]

Starting from N-(5-bromo-1-trityl-1H-indazol-3-yl)-1-methylpiperidine-4-carboxamide and (2,3-difluorophenyl)boronic acid. Flash column chromatography (DCM/MeOH/33% aqueous ammonia 90:10:0.5). Obtained 0.064 g (76% of yield). ¹H NMR (500 MHz, DMSO-d₆) δ ppm 1.59-1.71 (m, 2H), 1.78 (d, J=11.44 Hz, 2H), 1.85 (t, J=10.68 Hz, 2H), 2.15 (s, 3H), 2.42 (br. s., 1H), 2.80 (d, J=10.37 Hz, 2H), 6.47 (d, J=8.85 Hz, 1H), 7.23 (d, J=7.63 Hz, 6H), 7.26-7.32 (m, 6H), 7.32-7.38 (m, 6H), 7.41 (m, 1H), 7.91 (s, 1H), 10.50 (s, 1H). HRMS (ESI) calcd for C39H35F2N4O [M+H]⁺ 613.2774, found 613.2791.

### 1-Methyl-N-{5-[3-(methylsulfonyl)phenyl]-1-trityl-1H-indazol-3-yl}piperidine-4-carboxamide [(XI), R1=3-(methylsulfonyl)phenyl, R2=1-methylpiperidine, R9=1,1′,1″-methanetriyltribenzene]

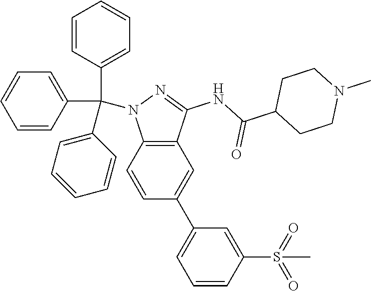 [SMILES: CN1CCC(C(=O)Nc2nn(C(c3ccccc3)(c3ccccc3)c3ccccc3)c3ccc(-c4cccc(S(C)(=O)=O)c4)cc23)CC1]

Starting from N-(5-bromo-1-trityl-1H-indazol-3-yl)-1-methylpiperidine-4-carboxamide and [3-(methylsulsonyl)phenyl]boronic acid. Flash column chromatography (DCM/MeOH 90:10). Obtained 0.036 g (43% of yield). ¹H NMR (500 MHz, DMSO-d₆) δ ppm 1.65-1.70 (m, 2H), 1.80-1.83 (m, J=11.74 Hz, 2H), 1.89-1.92 (m, 1H), 2.02-2.16 (m, 1H), 2.18 (br. s., 3H), 2.39-2.47 (m, 1H), 2.80-2.86 (m, 2H), 3.25 (s, 3H), 6.47 (d, J=9.00 Hz, 1H), 7.23 (d, J=7.47 Hz, 6H), 7.27-7.32 (m, 3H), 7.32-7.38 (m, 6H), 7.44 (d, J=8.54 Hz, 1H), 7.71 (t, J=7.78 Hz, 1H), 7.87 (m, 1H), 7.92 (d, J=7.63 Hz, 1H), 8.00 (s, 1H), 8.04 (br. s., 1H), 10.50 (br. s., 1H). HRMS (ESI) calcd for C40H39N4O3S [M+H]⁺ 655.2738, found 655.2762.

### N-[5-(3-Fluorophenyl)-1-trityl-1H-indazol-3-yl]-1-methylpiperidine-4-carboxamide [(XI), R1=3-fluorophenyl, R2=1-methylpiperidine, R9=1,1′,1″-methanetriyltribenzene]

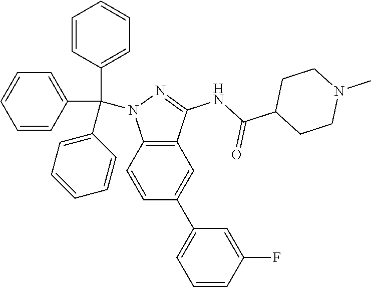 [SMILES: CN1CCC(C(=O)Nc2nn(C(c3ccccc3)(c3ccccc3)c3ccccc3)c3ccc(-c4cccc(F)c4)cc23)CC1]

Starting from N-(5-bromo-1-trityl-1H-indazol-3-yl)-1-methylpiperidine-4-carboxamide and (3-fluorophenyl)boronic acid. Flash column chromatography (DCM/MeOH 90:10). Obtained 0.054 g (70% of yield). ¹H NMR (500 MHz, DMSO-d₆) δ ppm 1.63-1.75 (m, 2H), 1.80-1.82 (m, 2H), 1.85-1.90 (m, 2H), 2.16 (br. s., 3H), 2.42 (m, 1H), 2.82 (m, J=8.39 Hz, 2H), 6.43 (d, J=8.85 Hz, 1H), 7.15 (td, J=8.92, 2.59 Hz, 1H), 7.20-7.25 (m, 6H), 7.27-7.31 (m, 3H), 7.32-7.36 (m, 7H), 7.37-7.43 (m, 2H), 7.45 (m, 1H), 7.97 (s, 1H), 10.47 (s, 1H). HRMS (ESI) calcd for C39H36FN4O [M+H]⁺ 595.2868, found 595.2891.

### N-[5-(2-Hydroxyphenyl)-1-trityl-1H-indazol-3-yl]-1-methylpiperidine-4-carboxamide [(XI), R1=2-hydroxyphenyl, R2=1-methylpiperidine, R9=1,1′,1″-methanetriyltribenzene]

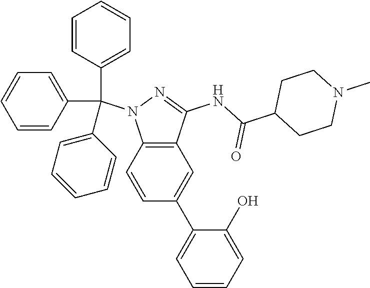 [SMILES: CN1CCC(C(=O)Nc2nn(C(c3ccccc3)(c3ccccc3)c3ccccc3)c3ccc(-c4ccccc4O)cc23)CC1]

Starting from N-(5-bromo-1-trityl-1H-indazol-3-yl)-1-methylpiperidine-4-carboxamide and (2-hydroxyphenyl)boronic acid. Flash column chromatography (DCM/MeOH 90:10). Obtained 0.061 g (78% of yield). LCMS (ESI) m/z 593 (M+H)⁺.

### N-[5-(Biphenyl-2-yl)-1-trityl-1H-indazol-3-yl]-1-methylpiperidine-4-carboxamide [(XI), R1=biphenyl-2-yl, R2=1-methylpiperidine, R9=1,1′,1″-methanetriyltribenzene]

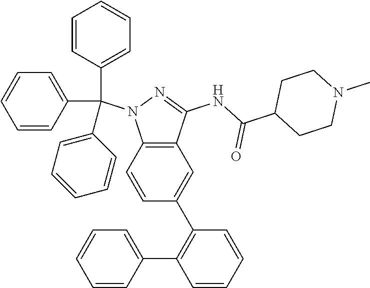 [SMILES: CN1CCC(C(=O)Nc2nn(C(c3ccccc3)(c3ccccc3)c3ccccc3)c3ccc(-c4ccccc4-c4ccccc4)cc23)CC1]

Starting from N-(5-bromo-1-trityl-1H-indazol-3-yl)-1-methylpiperidine-4-carboxamide and biphenyl-2-ylboronic acid. Flash column chromatography (DCM/MeOH 90:10). Obtained 0.064 g (74% of yield). ¹H NMR (500 MHz, DMSO-d₆) δ ppm 1.61-1.66 (m, J=9.76 Hz, 2H), 1.71-1.73 (m, 2H), 1.80-1.87 (m, 2H), 2.15 (br. s., 3H), 2.36 (m, 1H), 2.80 (m, J=10.22 Hz, 2H), 6.04 (d, J=8.85 Hz, 1H), 6.53 (d, J=8.54 Hz, 1H), 6.89-6.98 (m, 2H), 7.08-7.21 (m, 8H), 7.24-7.34 (m, 11H), 7.35-7.46 (m, 3H), 7.57 (s, 1H), 10.35 (s, 1H). HRMS (ESI) calcd for C45H₄₁N₄O [M+H]⁺ 653.3275, found 653.3276.

### 1-Methyl-N-{5-[2-(trifluoromethyl)phenyl]-1-trityl-1H-indazol-3-yl}piperidine-4-carboxamide [(XI), R1=2-(trifluoromethyl)phenyl, R2=1-methylpiperidine, R9=1,1′,1″-methanetriyltribenzene]

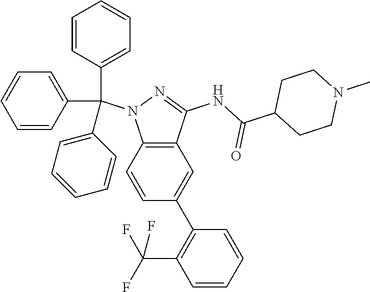 [SMILES: CN1CCC(C(=O)Nc2nn(C(c3ccccc3)(c3ccccc3)c3ccccc3)c3ccc(-c4ccccc4C(F)(F)F)cc23)CC1]

Starting from N-(5-bromo-1-trityl-1H-indazol-3-yl)-1-methylpiperidine-4-carboxamide and [2-(trifluromethyl)-phenyl]boronic acid. Flash column chromatography (DCM/MeOH 90:10). Obtained 0.038 g (99% of yield). LCMS (ESI) m/z 645 (M+H)⁺.

### N-[5-(2,6-Difluorophenyl)-1-trityl-1H-indazol-3-yl]-1-methylpiperidine-4-carboxamide [(XI), R1=2,6-difluorophenyl, R2=1-methylpiperidine, R9=1,1′,1″-methanetriyltribenzene]

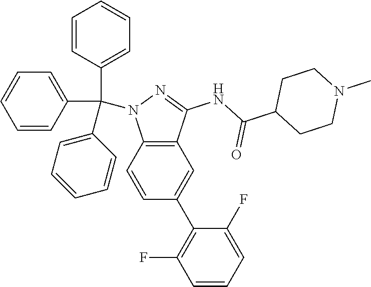 [SMILES: CN1CCC(C(=O)Nc2nn(C(c3ccccc3)(c3ccccc3)c3ccccc3)c3ccc(-c4c(F)cccc4F)cc23)CC1]

Starting from N-(5-bromo-1-trityl-1H-indazol-3-yl)-1-methylpiperidine-4-carboxamide and (2,6-difluorophenyl)boronic acid. Flash column chromatography (DCM/MeOH 90:10). Obtained 0.037 g (34% of yield). LCMS (ESI) m/z 613 (M+H)⁺.

### N-[5-(2,4-Dichlorophenyl)-1-trityl-1H-indazol-3-yl]-1-methylpiperidine-4-carboxamide [(XI), R1=2,4-dichlorophenyl, R2=1-methylpiperidine, R9=1,1′,1″-methanetriyltribenzene]

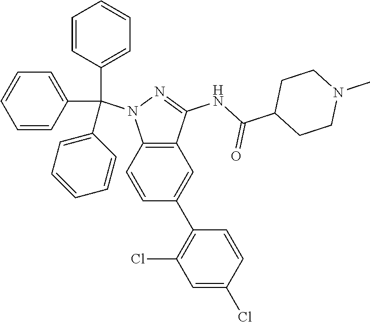 [SMILES: CN1CCC(C(=O)Nc2nn(C(c3ccccc3)(c3ccccc3)c3ccccc3)c3ccc(-c4ccc(Cl)cc4Cl)cc23)CC1]

Starting from N-(5-bromo-1-trityl-1H-indazol-3-yl)-1-methylpiperidine-4-carboxamide and (2,4-dichlorophenyl)boronic acid. Flash column chromatography (DCM/MeOH/33% aqueous ammonia 95:5:0.5). Obtained 0.080 g (61% of yield). ¹H NMR (500 MHz, DMSO-d₆) δ ppm 1.62-1.66 (m, 2H), 1.74-1.76 (m, 2H), 1.81-1.86 (m, 2H), 2.14 (s, 3H), 2.41 (m, 1H), 2.78 (d, J=10.37 Hz, 2H), 6.44 (d, J=9.00 Hz, 1H), 7.12 (d, J=8.85 Hz, 1H), 7.24 (d, J=7.63 Hz, 6H), 7.27-7.31 (m, 3H), 7.32-7.37 (m, 6H), 7.39 (d, J=8.39 Hz, 1H), 7.46 (m, 1H), 7.70 (d, J=2.13 Hz, 1H), 7.78 (s, 1H), 10.48 (s, 1H). HRMS (ESI) calcd for C39H35Cl2N4O [M+H]⁺ 645.2183, found 645.2186.

### N-[5-(4-Amino-2-chlorophenyl)-1-trityl-1H-indazol-3-yl]-1-methylpiperidine-4-carboxamide [(XI), R1=4-amino-2-chlorophenyl, R2=1-methylpiperidine, R9=1,1′,1″-methanetriyltribenzene]

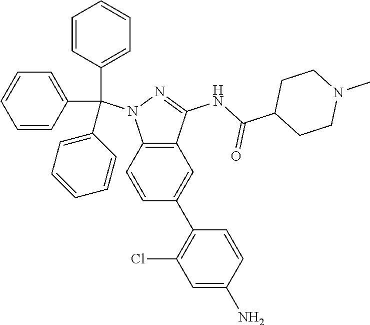 [SMILES: CN1CCC(C(=O)Nc2nn(C(c3ccccc3)(c3ccccc3)c3ccccc3)c3ccc(-c4ccc(N)cc4Cl)cc23)CC1]

Starting from N-(5-bromo-1-trityl-1H-indazol-3-yl)-1-methylpiperidine-4-carboxamide and 3-chloro-4-(4,4,5,5-tetramethyl-1,3,2-dioxaborolan-2-yl)aniline. Flash column chromatography (DCM/MeOH 90:10). Obtained 0.081 g (76% of yield). ¹H NMR (500 MHz, DMSO-d₆) δ ppm 1.67-1.73 (d, J=10.37 Hz, 3H), 1.82-1.85 (m, 2H), 2.27-2.33 (m, 2H), 2.57 (m, 1H), 2.89-3.01 (m, 2H), 5.45 (s, 2H), 6.37 (d, J=8.85 Hz, 1H), 6.54 (dd, J=8.31, 2.21 Hz, 1H), 6.66 (d, J=2.29 Hz, 1H), 6.98 (d, J=8.24 Hz, 1H), 7.03 (d, J=9.15 Hz, 1H), 7.23 (d, J=7.63 Hz, 6H), 7.27-7.30 (m, 3H), 7.32-7.39 (m, 6H), 7.63 (s, 1H), 10.45 (br. s., 1H). HRMS (ESI) calcd for C39H37ClN5O [M+H]⁺ 626.2681, found 626.2695.

### 1-Methyl-N-[5-(2,4,6-trichlorophenyl)-1-trityl-1H-indazol-3-yl]piperidine-4-carboxamide [(XI), R1=2,4,6-trichlorophenyl, R2=1-methylpiperidine, R9=1,1′,1″-methanetriyltribenzene]

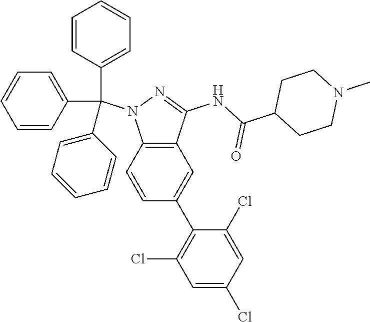 [SMILES: CN1CCC(C(=O)Nc2nn(C(c3ccccc3)(c3ccccc3)c3ccccc3)c3ccc(-c4c(Cl)cc(Cl)cc4Cl)cc23)CC1]

Starting from N-(5-bromo-1-trityl-1H-indazol-3-yl)-1-methylpiperidine-4-carboxamide and (2,4,6-trichlorophenyl)boronic acid. Flash column chromatography (DCM/MeOH/33% aqueous ammonia 95:5:0.5). Obtained 0.038 g (56% of yield). LCMS (ESI) m/z 679 (M+H)⁺.

### N-[5-(4-Carbamoylphenyl)-1-trityl-1H-indazol-3-yl]-1-methylpiperidine-4-carboxamide [(XI), R1=4-carbamoylphenyl, R2=1-methylpiperidine, R9=1,1′,1″-methanetriyltribenzene]

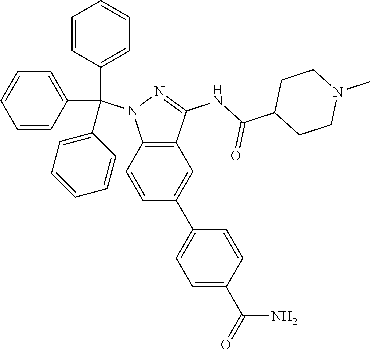 [SMILES: CN1CCC(C(=O)Nc2nn(C(c3ccccc3)(c3ccccc3)c3ccccc3)c3ccc(-c4ccc(C(N)=O)cc4)cc23)CC1]

Starting from N-(5-bromo-1-trityl-1H-indazol-3-yl)-1-methylpiperidine-4-carboxamide and (4-carbamoylphenyl)boronic acid. Flash column chromatography (DCM/MeOH/33% aqueous ammonia 90:10:1). Obtained 0.056 g (87% of yield). 1H NMR (500 MHz, DMSO-d₆) δ ppm 1.61-1.74 (m, 2H), 1.78-1.93 (m, 4H), 2.17 (br. s., 3H), 2.44 (m, 1H), 2.81-2.83 (m, 2H), 6.45 (d, J=8.85 Hz, 1H), 7.18-7.25 (m, 6H), 7.26-7.31 (m, 3H), 7.32-7.38 (m, 8H), 7.64 (d, J=8.08 Hz, 2H), 7.92 (d, J=8.39 Hz, 2H), 7.99 (br. s., 2H), 10.47 (s, 1H). HRMS (ESI) calcd for C40H38N5O2 [M+H]⁺ 620.3020, found 620.3024.

### N-[5-(4-Cyanophenyl)-1-trityl-1H-indazol-3-yl]-1-methylpiperidine-4-carboxamide [(XI), R1=4-cyanophenyl, R2=1-methylpiperidine, R9=1,1′,1″-methanetriyltribenzene]

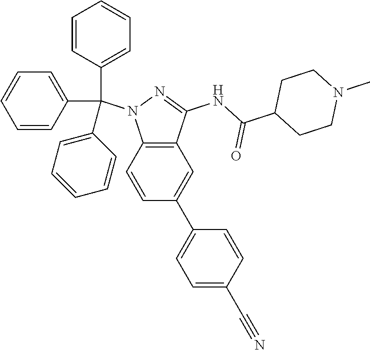 [SMILES: CN1CCC(C(=O)Nc2nn(C(c3ccccc3)(c3ccccc3)c3ccccc3)c3ccc(-c4ccc(C#N)cc4)cc23)CC1]

Starting from N-(5-bromo-1-trityl-1H-indazol-3-yl)-1-methylpiperidine-4-carboxamide and (4-cyanophenyl)boronic acid. Flash column chromatography (DCM/MeOH/33% aqueous ammonia 90:10:1). Obtained 0.059 g (97% of yield). ¹H NMR (500 MHz, DMSO-d₆) δ ppm 1.63-1.76 (m, 2H), 1.76-1.85 (m, 2H), 1.89-1.94 (m, 2H), 2.18 (br. s., 3H), 2.44 (m, 1H), 2.82-2.85 (m, 2H), 6.46 (d, J=9.00 Hz, 1H), 7.22 (d, J=7.47 Hz, 6H), 7.26-7.31 (m, 3H), 7.32-7.38 (m, 6H), 7.44 (d, J=8.54 Hz, 1H), 7.77 (d, J=8.24 Hz, 2H), 7.89 (d, J=8.54 Hz, 2H), 8.06 (s, 1H), 10.52 (s, 1H). HRMS (ESI) calcd for C40H36N5O [M+H]⁺ 602.2915, found 602.2923.

### N-[5-(2-Chloro-4-hydroxyphenyl)-1-trityl-1H-indazol-3-yl]-1-methylpiperidine-4-carboxamide [(XI), R1=2-chloro-4-hydroxyphenyl, R2=1-methylpiperidine, R9=1,1′,1″-methanetriyltribenzene]

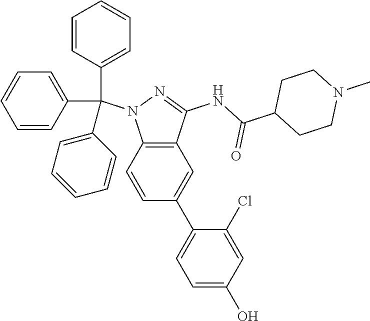 [SMILES: CN1CCC(C(=O)Nc2nn(C(c3ccccc3)(c3ccccc3)c3ccccc3)c3ccc(-c4ccc(O)cc4Cl)cc23)CC1]

Starting from N-(5-bromo-1-trityl-1H-indazol-3-yl)-1-methylpiperidine-4-carboxamide and (2-chloro-4-hydroxyphenyl)boronic acid. Flash column chromatography (DCM/MeOH 90:10). Obtained 0.046 g (73% of yield). LCMS (ESI) m/z 627 (M+H)⁺.

### N-[5-(4-Amino-3-fluorophenyl)-1-trityl-1H-indazol-3-yl]-1-methylpiperidine-4-carboxamide [(XI), R1=4-amino-3-fluorophenyl, R2=1-methylpiperidine, R9=1,1′,1″-methanetriyltribenzene]

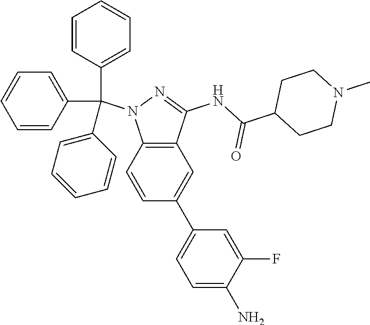 [SMILES: CN1CCC(C(=O)Nc2nn(C(c3ccccc3)(c3ccccc3)c3ccccc3)c3ccc(-c4ccc(N)c(F)c4)cc23)CC1]

Starting from N-(5-bromo-1-trityl-1H-indazol-3-yl)-1-methylpiperidine-4-carboxamide and 2-fluoro-4-(4,4,5,5-tetramethyl-1,3,2-dioxaborolan-2-yl)aniline. Flash column chromatography (DCM/MeOH 90:10). Obtained 0.050 g (81% of yield). LCMS (ESI) m/z 610 (M+H)⁺.

### N-[5-(4-Cyano-2-methylphenyl)-1-trityl-1H-indazol-3-yl]-1-methylpiperidine-4-carboxamide [(XI), R1=4-cyano-2-methylphenyl, R2=1-methylpiperidine, R9=1,1′,1″-methanetriyltribenzene]

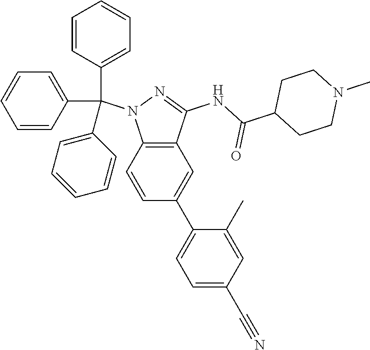 [SMILES: Cc1cc(C#N)ccc1-c1ccc2c(c1)c(NC(=O)C1CCN(C)CC1)nn2C(c1ccccc1)(c1ccccc1)c1ccccc1]

Starting from N-(5-bromo-1-trityl-1H-indazol-3-yl)-1-methylpiperidine-4-carboxamide and (4-cyano-2-methylphenyl)boronic acid. Flash column chromatography (DCM/MeOH/33% aqueous ammonia 90:10:0.5). Obtained 0.053 g (75% of yield). ¹H NMR (500 MHz, DMSO-d₆) δ ppm 1.61-1.67 (m, 2H), 1.74-1.78 (m, 2H), 1.82-2.00 (m, 2H), 2.17 (br. s., 3H), 2.22 (s, 3H), 2.41 (m, 1H), 2.78-2.82 (m, 2H), 6.44 (d, J=9.00 Hz, 1H), 7.10 (d, J=8.85 Hz, 1H), 7.24 (d, J=7.63 Hz, 6H), 7.27-7.31 (m, 3H), 7.32-7.38 (m, 7H), 7.68 (dd, J=7.93, 1.22 Hz, 1H), 7.71 (s, 1H), 7.77 (s, 1H), 10.50 (s, 1H). HRMS (ESI) calcd for C41H38N5O [M+H]⁺ 616.3071, found 616.3083.

### N-[5-(2,6-Dimethylphenyl)-1-trityl-1H-indazol-3-yl]-1-methylpiperidine-4-carboxamide [(XI), R1=2,6-dimethylphenyl, R2=1-methylpiperidine, R9=1,1′,1″-methanetriyltribenzene]

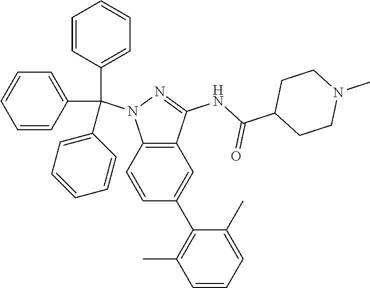 [SMILES: Cc1cccc(C)c1-c1ccc2c(c1)c(NC(=O)C1CCN(C)CC1)nn2C(c1ccccc1)(c1ccccc1)c1ccccc1]

Starting from N-(5-bromo-1-trityl-1H-indazol-3-yl)-1-methylpiperidine-4-carboxamide and (2,6-dimethylphenil)boronic acid. Flash column chromatography (DCM/MeOH/33% aqueous ammonia 95:5:0.5). Obtained 0.040 g (58% of yield). LCMS (ESI) m/z 605 (M+H)⁺.

### N-[5-(2-Fluoro-5-nitrophenyl)-1-trityl-1H-indazol-3-yl]-1-methylpiperidine-4-carboxamide [(XI), R1=2-fluoro-5-nitrophenyl, R2=1-methylpiperidine, R9=1,1′,1″-methanetriyltribenzene]

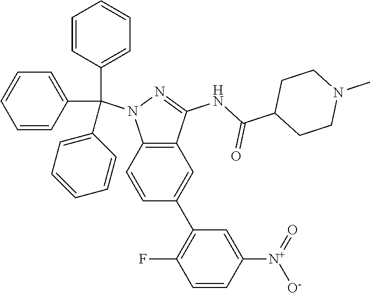 [SMILES: CN1CCC(C(=O)Nc2nn(C(c3ccccc3)(c3ccccc3)c3ccccc3)c3ccc(-c4cc([N+](=O)[O-])ccc4F)cc23)CC1]

Starting from N-(5-bromo-1-trityl-1H-indazol-3-yl)-1-methylpiperidine-4-carboxamide and (2-fluoro-5-nitrophenyl)boronic acid. Flash column chromatography (DCM/MeOH 90:10). Obtained 0.074 g (67% of yield). ¹H NMR (500 MHz, DMSO-d₆) δ ppm 1.68-1.71 (m, 2H), 1.79-1.82 (m, 2H), 1.89-1.96 (m, 2H), 2.19 (br. s., 3H), 2.45 (m, 1H), 2.82-2.87 (m, 2H), 6.50 (d, J=9.15 Hz, 1H), 7.23 (d, J=7.63 Hz, 6H), 7.27-7.32 (m, 4H), 7.33-7.39 (m, 6H), 7.61 (t, J=9.68 Hz, 1H), 8.00 (s, 1H), 8.23-8.30 (m, 2H), 10.55 (br. s., 1H). HRMS (ESI) calcd for C39H35FN5O3 [M+H]⁺ 640.2719, found 640.2732.

### N-[5-(2-Chloro-5-cyanophenyl)-1-trityl-1H-indazol-3-yl]-1-methylpiperidine-4-carboxamide [(XI), R1=2-chloro-5-cyanophenyl, R2=1-methylpiperidine, R9=1,1′,1″-methanetriyltribenzene]

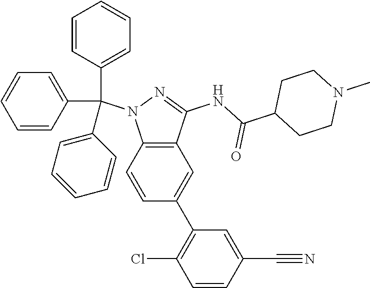 [SMILES: CN1CCC(C(=O)Nc2nn(C(c3ccccc3)(c3ccccc3)c3ccccc3)c3ccc(-c4cc(C#N)ccc4Cl)cc23)CC1]

Starting from N-(5-bromo-1-trityl-1H-indazol-3-yl)-1-methylpiperidine-4-carboxamide and (2-chloro-5-cyanophenil)boronic acid. Flash column chromatography (DCM/MeOH 90:10). Obtained 0.047 g (46% of yield). LCMS (ESI) m/z 636 (M+H)⁺.

### N-[5-(5-Carbamoyl-2-chlorophenyl)-1-trityl-1H-indazol-3-yl]-1-methylpiperidine-4-carboxamide [(XI), R1=5-carbamoyl-2-chlorophenyl, R2=1-methylpiperidine, R9=1,1′,1″-methanetriyltribenzene]

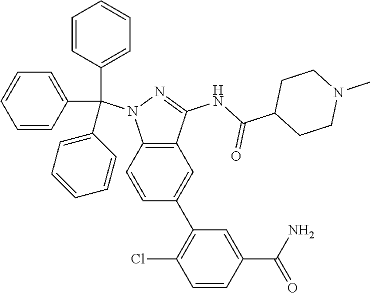 [SMILES: CN1CCC(C(=O)Nc2nn(C(c3ccccc3)(c3ccccc3)c3ccccc3)c3ccc(-c4cc(C(N)=O)ccc4Cl)cc23)CC1]

Starting from N-(5-bromo-1-trityl-1H-indazol-3-yl)-1-methylpiperidine-4-carboxamide and (5-carbamoyl-2-chlorophenyl)boronic acid. Flash column chromatography (DCM/MeOH 90:10). Obtained 0.085 g (49% of yield). LCMS (ESI) m/z 654 (M+H)⁺.

### N-[5-(2-Fluoro-4-formylphenyl)-1-trityl-1H-indazol-3-yl]-1-methylpiperidine-4-carboxamide [(XI), R1=2-fluoro-4-formylphenyl, R2=1-methylpiperidine, R9=1,1′,1″-methanetriyltribenzene]

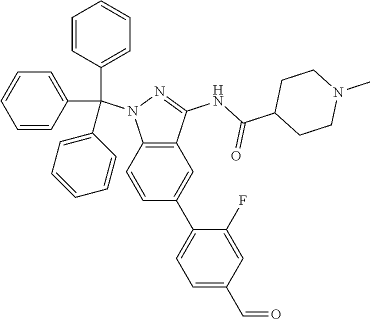 [SMILES: CN1CCC(C(=O)Nc2nn(C(c3ccccc3)(c3ccccc3)c3ccccc3)c3ccc(-c4ccc(C=O)cc4F)cc23)CC1]

Starting from N-(5-bromo-1-trityl-1H-indazol-3-yl)-1-methylpiperidine-4-carboxamide and (2-fluoro-4-formylphenyl)boronic acid. Flash column chromatography (DCM/MeOH 90:10). Obtained 0.040 g (39% of yield). LCMS (ESI) m/z 623 (M+H)⁺.

### N-[5-(2-Fluoro-4-methylphenyl)-1-trityl-1H-indazol-3-yl]-1-methylpiperidine-4-carboxamide [(XI), R1=2-fluoro-4-methylphenyl, R2=1-methylpiperidine, R9=1,1′,1″-methanetriyltribenzene]

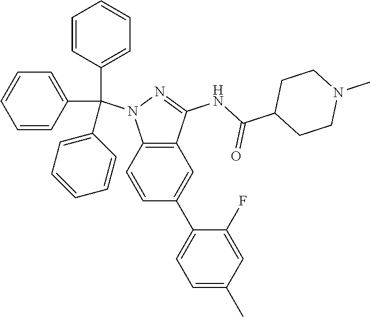 [SMILES: Cc1ccc(-c2ccc3c(c2)c(NC(=O)C2CCN(C)CC2)nn3C(c2ccccc2)(c2ccccc2)c2ccccc2)c(F)c1]

Starting from N-(5-bromo-1-trityl-1H-indazol-3-yl)-1-methylpiperidine-4-carboxamide and (2-fluoro-4-methylphenyl)boronic acid. Flash column chromatography (DCM/MeOH 90:10). Obtained 0.052 g (85% of yield). ¹H NMR (500 MHz, DMSO-d₆) δ ppm 1.58-1.72 (m, 2H), 1.77-1.80 (m, 2H), 1.86-1.92 (m, 2H), 2.17 (br. s., 3H), 2.33 (s, 3H), 2.41 (m, 1H), 2.81 (d, J=8.54 Hz, 2H), 6.43 (d, J=8.85 Hz, 1H), 7.08 (d, J=8.24 Hz, 1H), 7.10 (d, J=12.51 Hz, 1H), 7.18-7.36 (m, 17H), 7.82 (s, 1H), 10.45 (s, 1H). HRMS (ESI) calcd for C40H38FN4O [M+H]⁺ 609.3024, found 609.3027.

### N-[5-(2,6-Difluoro-4-methoxyphenyl)-1-trityl-1H-indazol-3-yl]-1-methylpiperidine-4-carboxamide [(XI), R1=2,6-difluoro-4-methoxyphenyl, R2=1-methylpiperidine, R9=1,1′,1″-methanetriyltribenzene]

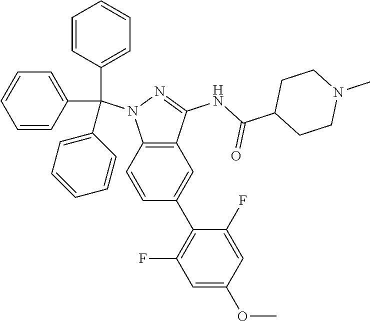 [SMILES: COc1cc(F)c(-c2ccc3c(c2)c(NC(=O)C2CCN(C)CC2)nn3C(c2ccccc2)(c2ccccc2)c2ccccc2)c(F)c1]

Starting from N-(5-bromo-1-trityl-1H-indazol-3-yl)-1-methylpiperidine-4-carboxamide and (2,6-difluoro-4-methoxyphenyl)boronic acid. Flash column chromatography (DCM/MeOH 90:10). Obtained 0.104 g (80% of yield). ¹H NMR (500 MHz, DMSO-d₆) δ ppm 1.55-1.71 (m, 2H), 1.75-1.77 (m, 2H), 1.82-1.89 (m, 2H), 2.15 (br. s., 3H), 2.40 (m, 1H), 2.79 (d, J=8.54 Hz, 2H), 3.80 (s, 3H), 6.45 (d, J=8.85 Hz, 1H), 6.83 (d, J=9.91 Hz, 2H), 7.06 (d, J=8.69 Hz, 1H), 7.23 (d, J=7.63 Hz, 6H), 7.27-7.30 (m, 3H), 7.33-7.36 (m, 6H), 7.75 (s, 1H), 10.47 (s, 1H). HRMS (ESI) calcd for C40H39FN4O [M+H]⁺ 643.2879, found 643.2887.

### tert-Butyl 4-(3-{[(1-methylpiperidin-4-yl)carbonyl]amino}-1-trityl-1H-indazol-5-yl)-1H-pyrazole-1-carboxylate [(XI), R1=tert-butyl 1H-pyrazole-1-carboxylate, R2=1-methylpiperidine, R9=methanetriyltribenzene]

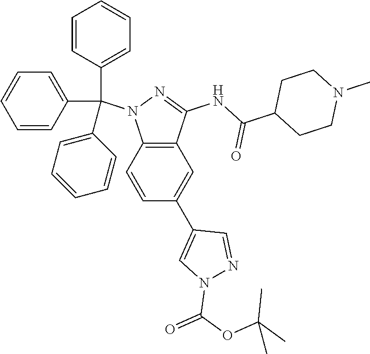 [SMILES: CN1CCC(C(=O)Nc2nn(C(c3ccccc3)(c3ccccc3)c3ccccc3)c3ccc(-c4cnn(C(=O)OC(C)(C)C)c4)cc23)CC1]

Starting from N-(5-bromo-1-trityl-1H-indazol-3-yl)-1-methylpiperidine-4-carboxamide and [1-(tert-butoxycarbonyl)-1H-pyrazol-4-yl]boronic acid. Flash column chromatography (DCM/MeOH 80:20). Obtained 0.056 g (81% of yield). LCMS (ESI) m/z 667 (M+H)⁺.

### N-[5-(2-Chloro-4-hydroxyphenyl)-1-trityl-1H-indazol-3-yl]-1-methylpiperidine-4-carboxamide [(XI), R1=2-chloro-4-hydroxyphenyl, R2=1-methylpiperidine, R9=1,1′,1″-methanetriyltribenzene]

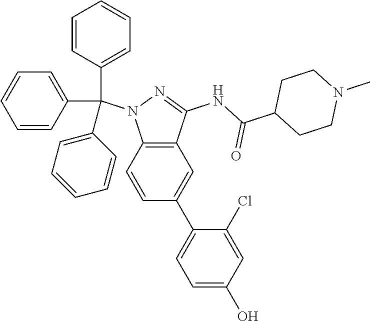 [SMILES: CN1CCC(C(=O)Nc2nn(C(c3ccccc3)(c3ccccc3)c3ccccc3)c3ccc(-c4ccc(O)cc4Cl)cc23)CC1]

Starting from N-(5-bromo-1-trityl-1H-indazol-3-yl)-1-methylpiperidine-4-carboxamide and (2-chloro-4-hydroxyphenyl)boronic acid. Flash column chromatography (DCM/MeOH/33% aqueous ammonia 90:10:0.5). Obtained 0.059 g (78% of yield). ¹H NMR (500 MHz, DMSO-d₆) δ ppm 1.63-1.72 (m, 2H), 1.75-1.82 (m, 2H), 1.83-1.88 (m, 2H), 2.15 (s, 3H), 2.41 (m, 1H), 2.80 (d, J=11.13 Hz, 2H), 6.40 (s, 1H), 6.72 (dd, J=8.39, 2.44 Hz, 1H), 6.80 (d, J=2.44 Hz, 1H), 6.90 (d, J=8.39 Hz, 1H), 7.00 (d, J=8.85 Hz, 1H), 7.19 (d, J=7.47 Hz, 6H), 7.24-7.28 (m, 3H), 7.29-7.34 (m, 6H), 7.68 (d, J=8.39 Hz, 1H), 9.98 (br. s., 1H), 10.42 (s, 1H). HRMS (ESI) calcd for C39H36ClN4O2 [M+H]⁺ 627.2522, found 627.2529.

### 1-Methyl-N-[5-(1-methyl-1H-pyrazol-4-yl)-1-trityl-1H-indazol-3-yl]piperidine-4-carboxamide [(XI), R1=1-methyl-1H-pyrazol-4-yl, R2=1-methylpiperidine, R9=1,1′,1″-methanetriyltribenzene]

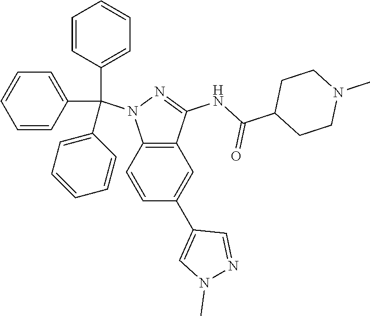 [SMILES: CN1CCC(C(=O)Nc2nn(C(c3ccccc3)(c3ccccc3)c3ccccc3)c3ccc(-c4cnn(C)c4)cc23)CC1]

Starting from N-(5-bromo-1-trityl-1H-indazol-3-yl)-1-methylpiperidine-4-carboxamide and 1-methyl-4-(4,4,5,5-tetramethyl-1,3,2-dioxaborolan-2-yl)-1H-pyrazole. Flash column chromatography (DCM/MeOH 85:15). Obtained 0.060 g (95% of yield). LCMS (ESI) m/z 581 (M+H)⁺.

### 1-Methyl-N-[5-(2,4,6-trifluorophenyl)-1-trityl-1H-indazol-3-yl]piperidine-4-carboxamide [(XI), R1=2,4,6-trifluorophenyl, R2=1-methylpiperidine, R9=1,1′,1″-methanetriyltribenzene]

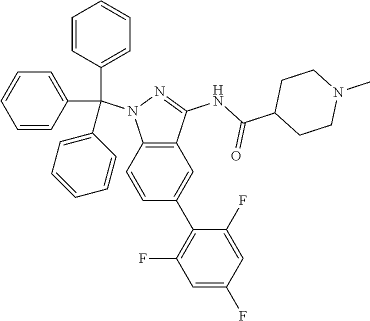 [SMILES: CN1CCC(C(=O)Nc2nn(C(c3ccccc3)(c3ccccc3)c3ccccc3)c3ccc(-c4c(F)cc(F)cc4F)cc23)CC1]

Starting from N-(5-bromo-1-trityl-1H-indazol-3-yl)-1-methylpiperidine-4-carboxamide and (2,4,6-trifluorophenyl)boronic acid. Flash column chromatography (DCM/MeOH 90:10). Obtained 0.011 g (17% of yield). LCMS (ESI) m/z 631 (M+H)⁺.

### N-[5-(2,4-Difluorophenyl)-1-trityl-1H-indazol-3-yl]-1-methylpiperidine-4-carboxamide [(XI), R1=2,4-difluorophenyl, R2=1-methylpiperidine, R9=1,1′,1″-methanetriyltribenzene]

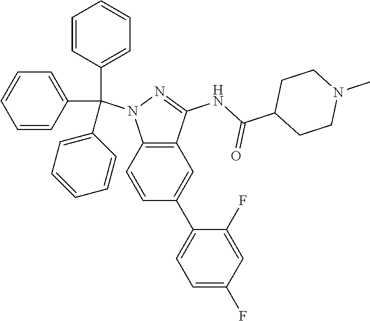 [SMILES: CN1CCC(C(=O)Nc2nn(C(c3ccccc3)(c3ccccc3)c3ccccc3)c3ccc(-c4ccc(F)cc4F)cc23)CC1]

Starting from N-(5-bromo-1-trityl-1H-indazol-3-yl)-1-methylpiperidine-4-carboxamide and (2,4-difluorophenyl)boronic acid. Flash column chromatography (DCM/MeOH 90:10). Obtained 0.044 g (72% of yield). ¹H NMR (500 MHz, DMSO-d₆) δ ppm 1.62-1.68 (m, 2H), 1.77-1.79 (m, 2H), 1.84-1.94 (m, 1H), 2.17 (br. s., 3H), 2.43 (m, 1H), 2.82 (d, J=8.85 Hz, 2H), 6.45 (d, J=8.85 Hz, 1H), 7.14-7.19 (m, 2H), 7.22 (d, J=7.63 Hz, 6H), 7.27-7.30 (m, 4H), 7.33-7.36 (m, 6H), 7.49 (m, 1H), 7.84 (s, 1H), 10.48 (s, 1H). HRMS (ESI) calcd for C39H35F2N4O [M+H]⁺ 613.2774, found 613.2773.

### 1-Methyl-N-[5-(1H-pyrazol-3-yl)-1-trityl-1H-indazol-3-yl]piperidine-4-carboxamide [(XI), R1=1H-pyrazol-3-yl, R2=1-methylpiperidine, R9=1,1′,1″-methanetriyltribenzene]

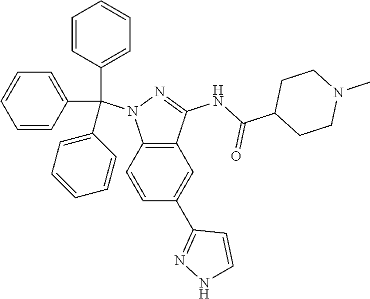 [SMILES: CN1CCC(C(=O)Nc2nn(C(c3ccccc3)(c3ccccc3)c3ccccc3)c3ccc(-c4cc[nH]n4)cc23)CC1]

Starting from N-(5-bromo-1-trityl-1H-indazol-3-yl)-1-methylpiperidine-4-carboxamide and 1H-pyrazol-3-ylboronic acid. Flash column chromatography (DCM/MeOH/33% aqueous ammonia 90:10:1). Obtained 0.040 g (69% of yield). LCMS (ESI) m/z 567 (M+H)⁺.

### 1-Methyl-N-[5-(2,4,5-trifluorophenyl)-1-trityl-1H-indazol-3-yl]piperidine-4-carboxamide [(XI), R1=2,4,5-trifluorophenyl, R2=1-methylpiperidine, R9=1,1′,1″-methanetriyltribenzene]

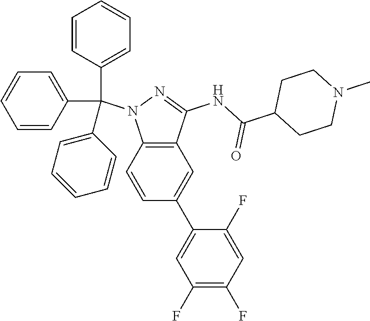 [SMILES: CN1CCC(C(=O)Nc2nn(C(c3ccccc3)(c3ccccc3)c3ccccc3)c3ccc(-c4cc(F)c(F)cc4F)cc23)CC1]

Starting from N-(5-bromo-1-trityl-1H-indazol-3-yl)-1-methylpiperidine-4-carboxamide and (2,4,5-trifluorophenyl)boronic acid. Flash column chromatography (DCM/MeOH 90:10). Obtained 0.039 g (62% of yield). ¹H NMR (500 MHz, DMSO-d₆) δ ppm 1.67-1.72 (m, 2H), 1.80-1.85 (m, 2H), 1.93-2.02 (m, 2H), 2.24 (br. s., 3H), 2.45 (m, 1H), 2.85-2.94 (m, 2H), 6.45 (d, J=9.00 Hz, 1H), 7.21 (d, J=7.63 Hz, 6H), 7.28-7.30 (m, 4H), 7.33-7.36 (m, 6H), 7.60-7.67 (m, 2H), 7.88 (s, 1H), 10.52 (br. s., 1H). HRMS (ESI) calcd for C39H34F3N4O [M+H]⁺ 631.2679, found 631.2680.

### 1-Methyl-N-{5-[2,4,6-trifluoro-3-(propan-2-yloxy)phenyl]-1-trityl-1H-indazol-3-yl}piperidine-4-carboxamide [(XI), R1=2,4,6-trifluoro-3-(propan-2-yloxy)phenyl, R2=1-methylpiperidine, R9=methanetriyltribenzene]

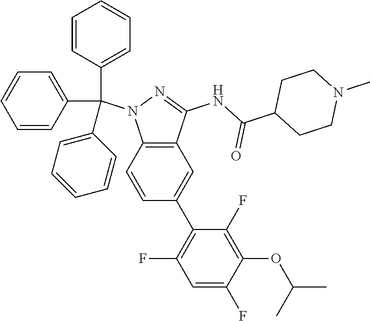 [SMILES: CC(C)Oc1c(F)cc(F)c(-c2ccc3c(c2)c(NC(=O)C2CCN(C)CC2)nn3C(c2ccccc2)(c2ccccc2)c2ccccc2)c1F]

Starting from N-(5-bromo-1-trityl-1H-indazol-3-yl)-1-methylpiperidine-4-carboxamide and [2,4,6-trifluoro-3-(propan-2-yloxy)phenyl]boronic acid. Flash column chromatography (DCM/MeOH 90:10). Obtained 0.034 g (50% of yield). ¹H NMR (500 MHz, DMSO-d₆) δ ppm 1.26 (d, J=6.10 Hz, 6H), 1.65-1.70 (m, 2H), 1.79-1.82 (m, 2H), 1.99-2.07 (m, 2H), 2.24 (br. s., 3H), 2.46 (m, 1H), 2.86-2.93 (m, 2H), 4.30 (spt, J=6.13 Hz, 1H), 6.47 (d, J=9.00 Hz, 1H), 7.10 (d, J=8.85 Hz, 1H), 7.23 (d, J=7.78 Hz, 6H), 7.28-7.30 (m, 4H), 7.33-7.36 (m, 6H), 7.81 (s, 1H), 10.53 (br. s., 1H). HRMS (ESI) calcd for C42H40F3N4O2 [M+H]⁺ 689.3098, found 689.3083.

### 1-Methyl-N-[5-(2,3,4-trifluorophenyl)-1-trityl-1H-indazol-3-yl]piperidine-4-carboxamide [(XI), R1=2,3,4-trifluorophenyl, R2=1-methylpiperidine, R9=1,1′,1″-methanetriyltribenzene]

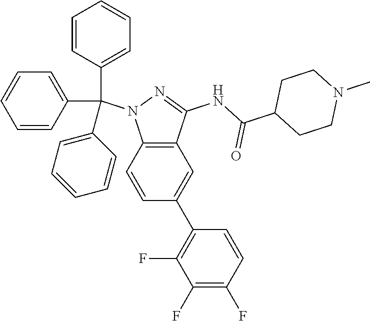 [SMILES: CN1CCC(C(=O)Nc2nn(C(c3ccccc3)(c3ccccc3)c3ccccc3)c3ccc(-c4ccc(F)c(F)c4F)cc23)CC1]

Starting from N-(5-bromo-1-trityl-1H-indazol-3-yl)-1-methylpiperidine-4-carboxamide and (2,3,4-trifluorophenyl)boronic acid. Flash column chromatography (DCM/MeOH 90:10). Obtained 0.091 g (35% of yield). ¹H NMR (500 MHz, DMSO-d₆) δ ppm 1.59-1.70 (m, 2H), 1.76-1.79 (m, 2H), 1.82-1.89 (m, 2H), 2.15 (br. s., 3H), 2.42 (m, 1H), 2.80 (d, J=10.07 Hz, 2H), 6.47 (d, J=9.00 Hz, 1H), 7.22 (d, J=7.63 Hz, 6H), 7.27-7.30 (m, 4H), 7.33-7.36 (m, 8H), 7.89 (s, 1H), 10.50 (s, 1H). HRMS (ESI) calcd for C39H34F3N4O [M+H]⁺ 631.2679, found 631.2680.

### N-[5-(4-Fluoro-2-methylphenyl)-1-trityl-1H-indazol-3-yl]-1-methylpiperidine-4-carboxamide [(XI), R1=4-fluoro-2-methylphenyl, R2=1-methylpiperidine, R9=1,1′,1″-methanetriyltribenzene]

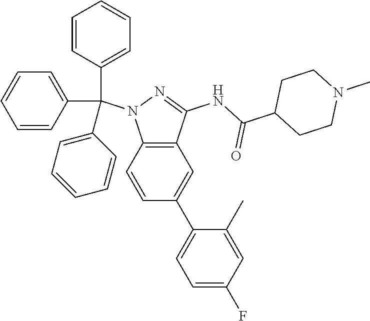 [SMILES: Cc1cc(F)ccc1-c1ccc2c(c1)c(NC(=O)C1CCN(C)CC1)nn2C(c1ccccc1)(c1ccccc1)c1ccccc1]

Starting from N-(5-bromo-1-trityl-1H-indazol-3-yl)-1-methylpiperidine-4-carboxamide and (4-fluoro-2-methylphenyl)boronic acid. Flash column chromatography (DCM/MeOH 90:10). Obtained 0.077 g (71% of yield). ¹H NMR (500 MHz, DMSO-d₆) δ ppm 1.63-1.67 (m, 2H), 1.76-1.78 (m, 2H), 1.87-1.93 (m, 2H), 2.16-2.19 (m, 6H), 2.41 (m, 1H), 2.81-2.84 (m, 2H), 6.41 (d, J=9.00 Hz, 1H), 7.01-7.07 (m, 2H), 7.13 (dd, J=10.14, 2.67 Hz, 1H), 7.17 (dd, J=8.39, 6.25 Hz, 1H), 7.24 (d, J=7.63 Hz, 6H), 7.27-7.31 (m, 3H), 7.32-7.37 (m, 6H), 7.61 (s, 1H), 10.45 (s, 1H). HRMS (ESI) calcd for C40H38FN4O [M+H]⁺ 609.3024, found 609.3029.

### N-[5-(4-Amino-2-methylphenyl)-1-trityl-1H-indazol-3-yl]-1-methylpiperidine-4-carboxamide [(XI), R1=4-amino-2-methylphenyl, R2=1-methylpiperidine, R9=1,1′,1″-methanetriyltribenzene]

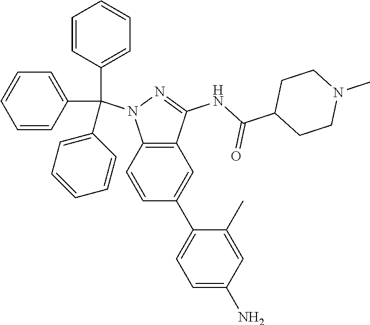 [SMILES: Cc1cc(N)ccc1-c1ccc2c(c1)c(NC(=O)C1CCN(C)CC1)nn2C(c1ccccc1)(c1ccccc1)c1ccccc1]

Starting from N-(5-bromo-1-trityl-1H-indazol-3-yl)-1-methylpiperidine-4-carboxamide and (4-ammino-2-methylphenyl)boronic acid. Flash column chromatography (DCM/MeOH 85:15). Obtained 0.046 g (61% of yield). 1H NMR (500 MHz, DMSO-d₆) δ ppm 1.58-1.70 (m, 2H), 1.74-1.79 (m, 2H), 1.85-1.89 (m, 2H), 2.04 (s, 3H), 2.16 (br. s., 3H), 2.41 (m, 1H), 2.79-2.81 (m, 2H), 5.01 (s, 2H), 6.35 (d, J=8.85 Hz, 1H), 6.41 (dd, J=8.16, 2.21 Hz, 1H), 6.44 (d, J=2.14 Hz, 1H), 6.79 (d, J=8.24 Hz, 1H), 6.95 (d, J=8.69 Hz, 1H), 7.24 (d, J=7.63 Hz, 6H), 7.25-7.30 (m, 3H), 7.31-7.37 (m, 6H), 7.49 (s, 1H), 10.36 (s, 1H). HRMS (ESI) calcd for C40H40N5O [M+H]⁺ 606.3228, found 606.3224.

### 1-Methyl-N-[5-(2,4,6-trifluoro-3-methoxyphenyl)-1-trityl-1H-indazol-3-yl]piperidine-4-carboxamide [(XI), R1=2,4,6-trifluoro-3-methoxyphenyl, R2=1-methylpiperidine, R9=1,1′,1″-methanetriyltribenzene]

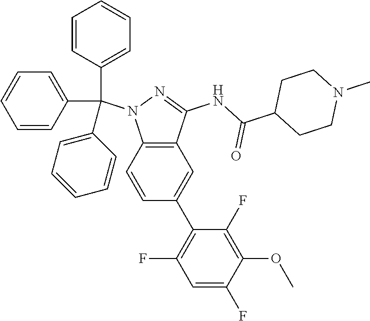 [SMILES: COc1c(F)cc(F)c(-c2ccc3c(c2)c(NC(=O)C2CCN(C)CC2)nn3C(c2ccccc2)(c2ccccc2)c2ccccc2)c1F]

Starting from N-(5-bromo-1-trityl-1H-indazol-3-yl)-1-methylpiperidine-4-carboxamide and (2,4,6-trifluoro-3-methoxyphenyl)boronic acid. Flash column chromatography (DCM/MeOH 95:5). Obtained 0.055 g (61% of yield). ¹H NMR (500 MHz, DMSO-d₆) δ ppm 1.59-1.71 (m, 2H), 1.76-1.79 (m, 2H), 1.87-1.96 (m, 2H), 2.18 (br. s., 3H), 2.43 (m, 1H), 2.83 (d, J=2.90 Hz, 2H), 3.89 (s, 3H), 6.47 (d, J=8.85 Hz, 1H), 7.09 (d, J=8.69 Hz, 1H), 7.23 (d, J=7.78 Hz, 6H), 7.26-7.31 (m, 4H), 7.32-7.42 (m, 7H), 7.80 (s, 1H), 10.51 (s, 1H). HRMS (ESI) calcd for C40H36F3N4O2 [M+H]⁺ 661.2785, found 661.2786.

### N-[5-(2,3-Difluoro-4-methylphenyl)-1-trityl-1H-indazol-3-yl]-1-methylpiperidine-4-carboxamide [(XI), R1=2,3-difluoro-4-methylphenyl, R2=1-methylpiperidine, R9=1,1′,1″-methanetriyltribenzene]

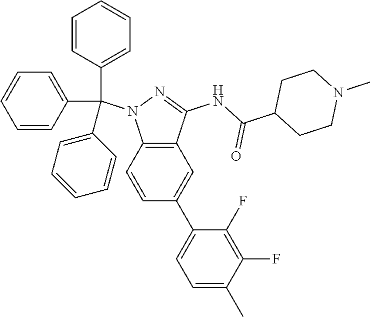 [SMILES: Cc1ccc(-c2ccc3c(c2)c(NC(=O)C2CCN(C)CC2)nn3C(c2ccccc2)(c2ccccc2)c2ccccc2)c(F)c1F]

Starting from N-(5-bromo-1-trityl-1H-indazol-3-yl)-1-methylpiperidine-4-carboxamide and (2,3-difluoro-4-methylphenyl)boronic acid. Flash column chromatography (DCM/MeOH 90:10). Obtained 0.050 g (77% of yield). LCMS (ESI) m/z 627 (M+H)⁺.

### N-[5-(2-Chloro-4-fluoro-3-methylphenyl)-1-trityl-1H-indazol-3-yl]-1-methylpiperidine-4-carboxamide [(XI), R1=2-chloro-4-fluoro-3-methylphenyl, R2=1-methylpiperidine, R9=1,1′,1″-methanetriyltribenzene]

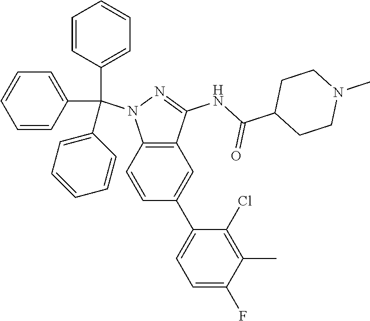 [SMILES: Cc1c(F)ccc(-c2ccc3c(c2)c(NC(=O)C2CCN(C)CC2)nn3C(c2ccccc2)(c2ccccc2)c2ccccc2)c1Cl]

Starting from N-(5-bromo-1-trityl-1H-indazol-3-yl)-1-methylpiperidine-4-carboxamide and (2-chloro-4-fluoro-3-methylphenyl)boronic acid. Flash column chromatography (DCM/MeOH 90:10). Obtained 0.058 g (48% of yield). LCMS (ESI) m/z 643 (M+H)⁺.

### N-[5-(3-Cyano-2,6-difluorophenyl)-1-trityl-1H-indazol-3-yl]-1-methylpiperidine-4-carboxamide [(XI), R1=3-cyano-2,6-difluorophenyl, R2=1-methylpiperidine, R9=1,1′,1″-methanetriyltribenzene]

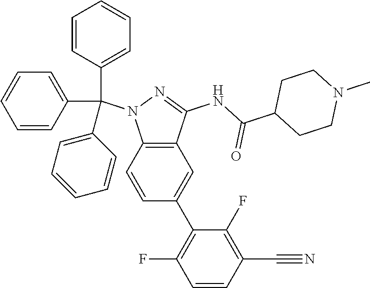 [SMILES: CN1CCC(C(=O)Nc2nn(C(c3ccccc3)(c3ccccc3)c3ccccc3)c3ccc(-c4c(F)ccc(C#N)c4F)cc23)CC1]

Starting from N-(5-bromo-1-trityl-1H-indazol-3-yl)-1-methylpiperidine-4-carboxamide and (3-cyano-2,6-difluorophenyl)boronic acid. Flash column chromatography (DCM/MeOH 95:5). Obtained 0.030 g (45% of yield). LCMS (ESI) m/z 638 (M+H)⁺.

### N-[5-(3-Cyano-4-hydroxyphenyl)-1-trityl-1H-indazol-3-yl]-1-methylpiperidine-4-carboxamide [(XI), R1=3-cyano-4-hydroxyphenyl, R2=1-methylpiperidine, R9=1,1′,1″-methanetriyltribenzene]

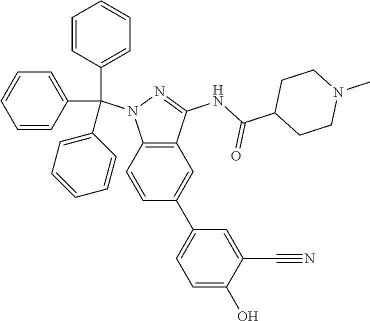 [SMILES: CN1CCC(C(=O)Nc2nn(C(c3ccccc3)(c3ccccc3)c3ccccc3)c3ccc(-c4ccc(O)c(C#N)c4)cc23)CC1]

Starting from N-(5-bromo-1-trityl-1H-indazol-3-yl)-1-methylpiperidine-4-carboxamide and 2-hydroxy-5-(4,4,5,5-tetramethyl-1,3,2-dioxaborolan-2-yl)benzonitrile. Flash column chromatography (DCM/MeOH/33% aqueous ammonia 90:10:0.5). Obtained 0.047 g (87% of yield). ¹H NMR (500 MHz, DMSO-d₆) δ ppm 1.66-1.89 (m, 6H), 2.16 (s, 3H), 2.42 (m, 1H), 2.82 (d, J=10.98 Hz, 2H), 6.39 (d, J=8.85 Hz, 1H), 7.05 (d, J=8.85 Hz, 1H), 7.21 (d, J=7.63 Hz, 6H), 7.26-7.31 (m, 4H), 7.31-7.39 (m, 6H), 7.68 (d, J=8.24 Hz, 1H), 7.75 (br. s., 1H), 7.85 (s, 1H), 10.41 (s, 1H). HRMS (ESI) calcd for C40H37F3N4O2 [M+H]⁺ 618.2864, found 618.2874.

### N-[5-(2-Cyano-5-fluorophenyl)-1-trityl-1H-indazol-3-yl]-1-methylpiperidine-4-carboxamide [(XI), R1=2-cyano-5-fluorophenyl, R2=1-methylpiperidine, R9=1,1′,1″-methanetriyltribenzene]

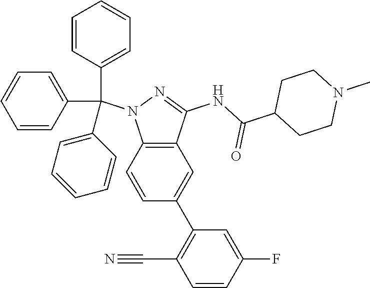 [SMILES: CN1CCC(C(=O)Nc2nn(C(c3ccccc3)(c3ccccc3)c3ccccc3)c3ccc(-c4cc(F)ccc4C#N)cc23)CC1]

Starting from N-(5-bromo-1-trityl-1H-indazol-3-yl)-1-methylpiperidine-4-carboxamide and (2-cyano-5-fluorophenyl)boronic acid. Flash column chromatography (DCM/MeOH 90:10). Obtained 0.053 g (93% of yield). ¹H NMR (500 MHz, DMSO-d₆) δ ppm 1.66-1.72 (m, 2H), 1.78-1.81 (m, 2H), 1.91-2.01 (m, 2H), 2.20 (br. s., 3H), 2.44 (m, 1H), 2.82-2.87 (m, 2H), 6.49 (d, J=9.00 Hz, 1H), 7.24 (d, J=7.63 Hz, 6H), 7.28-7.32 (m, 4H), 7.33-7.38 (m, 6H), 7.43 (td, J=8.39, 2.59 Hz, 1H), 7.49 (m, 1H), 7.98-8.05 (m, 2H), 10.57 (s, 1H). HRMS (ESI) calcd for C40H35FN5O [M+H]⁺ 620.2820, found 620.2830.

### N-[5-(2-Chloro-5-nitrophenyl)-1-trityl-1H-indazol-3-yl]-1-methylpiperidine-4-carboxamide [(XI), R1=2-chloro-5-nitrophenyl, R2=1-methylpiperidine, R9=1,1′,1″-methanetriyltribenzene]

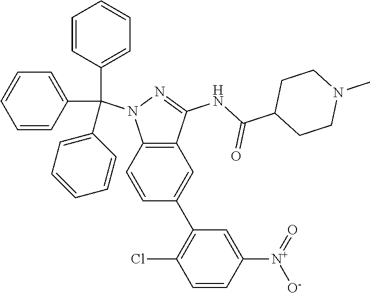 [SMILES: CN1CCC(C(=O)Nc2nn(C(c3ccccc3)(c3ccccc3)c3ccccc3)c3ccc(-c4cc([N+](=O)[O-])ccc4Cl)cc23)CC1]

Starting from N-(5-bromo-1-trityl-1H-indazol-3-yl)-1-methylpiperidine-4-carboxamide and (2-chloro-5-nitrophenyl)boronic acid. Flash column chromatography (DCM/MeOH 90:10). Obtained 0.040 g (61% of yield). LCMS (ESI) m/z 656 (M+H)⁺.

### N-[5-(2-Chloro-4-hydroxyphenyl)-1-trityl-1H-indazol-3-yl]-3-(dimethylamino)cyclobutanecarboxamide [(XI), R1=2-chloro-4-hydroxyphenyl, R2=(dimethylamino)cyclobutane, R9=1,1′,1″-methanetriyltribenzene]

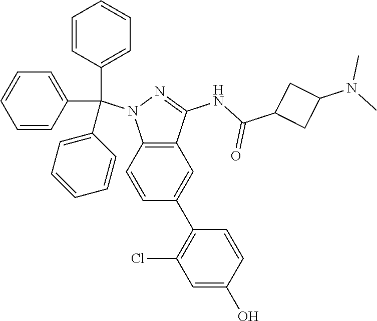 [SMILES: CN(C)C1CC(C(=O)Nc2nn(C(c3ccccc3)(c3ccccc3)c3ccccc3)c3ccc(-c4ccc(O)cc4Cl)cc23)C1]

Starting from N-(5-bromo-1-trityl-1H-indazol-3-yl)-3-(dimethylamino)cyclobutanecarboxamide and (2-chloro-4-hydroxyphenyl)boronic acid. Flash column chromatography (DCM/MeOH/33% aqueous ammonia 95:10:0.5). Obtained 0.050 g (54% of yield). ¹H NMR (500 MHz, DMSO-d₆) δ ppm 1.94-2.01 (m, 8H), 2.19 (m, 2H), 2.54 (m, 1H), 2.92 (m, 1H), 6.40 (d, J=8.85 Hz, 1H), 6.78 (dd, J=8.46, 2.52 Hz, 1H), 6.89 (d, J=2.44 Hz, 1H), 7.06 (d, J=8.85 Hz, 1H), 7.16 (d, J=8.39 Hz, 1H), 7.24 (d, J=7.63 Hz, 6H), 7.26-7.30 (m, 3H), 7.32-7.38 (m, 6H), 7.70 (s, 1H), 9.96 (s, 1H), 10.43 (s, 1H). HRMS (ESI) calcd for C39H36ClN4O2 [M+H]⁺ 627.2522, found 627.2520.

### N-[5-(2-Chloro-4-hydroxyphenyl)-1-trityl-1H-indazol-3-yl]-3-(methylamino)cyclobutanecarboxamide [(XI), R1=2-chloro-4-hydroxyphenyl, R2=(methylamino)cyclobutane, R9=1,1′,1″-methanetriyltribenzene]

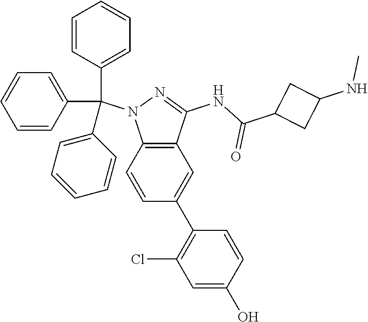 [SMILES: CNC1CC(C(=O)Nc2nn(C(c3ccccc3)(c3ccccc3)c3ccccc3)c3ccc(-c4ccc(O)cc4Cl)cc23)C1]

Starting from N-(5-bromo-1-trityl-1H-indazol-3-yl)-3-(methylamino)cyclobutanecarboxamide and (2-chloro-4-hydroxyphenyl)boronic acid. Flash column chromatography (DCM/MeOH/33% aqueous ammonia 90:10:0.5). Obtained 0.059 g (60% of yield). LCMS (ESI) m/z 613 (M+H)⁺.

### tert-Butyl (3R)-3-({5-[4-amino-2-chloro-5-(methoxycarbonyl)phenyl]-1-trityl-1H-indazol-3-yl}carbamoyl)-pyrrolidine-1-carboxylate [(XI), R1=4-amino-2-chloro-5-(methoxycarbonyl)phenyl, R2=tert-butyl pyrrolidine-1-carboxylate, R9=1,1′,1″-methanetriyltribenzene]

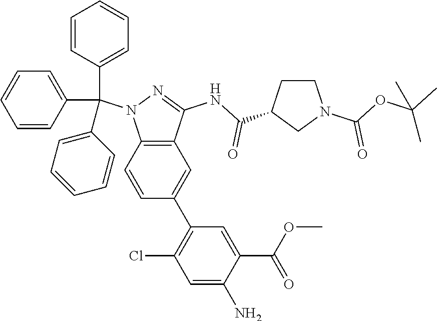 [SMILES: COC(=O)c1cc(-c2ccc3c(c2)c(NC(=O)[C@@H]2CCN(C(=O)OC(C)(C)C)C2)nn3C(c2ccccc2)(c2ccccc2)c2ccccc2)c(Cl)cc1N]

A solution of tert-butyl (3R)-3-{[5-(4,4,5,5-tetramethyl-1,3,2-dioxaborolan-2-yl)-1-trityl-1H-indazol-3-yl]carbamoyl}-pyrrolidine-1-carboxylate (0.070 g, 0.10 mmol) in mixture 1,4-dioxane/water 3:1 (4 mL) was evacuated with argon for 5 min. Methyl 2-amino-5-bromo-4-chlorobenzoate (0.053 g, 0.20 mmol), 1,1′-Bis(diphenylphosphino)-ferrocene]dichloropalladium(II)×DCM (10% mol, 0.0082 g, 0.01 mmol) and Cs₂CO₃ (0.064 g, 0.20 mmol) were added. The mixture was heated, under argon, at 80° C., for 1 h, then was cooled, diluted with EtOAc (20 mL) and washed with aqueous NaHCO₃ (20 mL), water (20 mL) and brine (20 mL). The organic phase was separated, dried with anhydrous Na₂SO₄, filtered and evaporated to dryness. The crude was purified by flash column chromatography (Hexane/EtOAc 65:35) to obtain tert-butyl (3R)-3-({5-[4-amino-2-chloro-5-(methoxycarbonyl)phenyl]-1-trityl-1H-indazol-3-yl)carbamoyl)pyrrolidine-1-carboxylate (0.064 g, 85% of yield) as white solid. ¹H NMR (500 MHz, DMSO-d₆) δ ppm 1.37 (s, 9H), 1.98-2.19 (m, 2H), 3.13-3.30 (m, 2H), 3.36-3.43 (m, 2H), 3.46-3.57 (m, 1H), 3.76 (s, 3H), 6.40 (d, J=9.00 Hz, 1H), 6.85 (s, 2H), 6.96 (s, 1H), 7.03 (d, J=9.15 Hz, 1H), 7.22 (d, J=7.63 Hz, 6H), 7.27-7.30 (m, 3H), 7.33-7.36 (m, 6H), 7.59 (s, 1H), 7.67 (s, 1H), 10.65 (d, J=8.24 Hz, 1H). HRMS (ESI) calcd for C44H43ClN5O5Na [M+H+Na]⁺778.2766, found 778.2772.

According to this same methodology, but employing suitable intermediates and reagents, the following compounds were prepared:

### tert-Butyl (3R)-3-({5-[2-cyano-5-(methylcarbamoyl)phenyl]-1-trityl-1H-indazol-3-yl}carbamoyl)piperidine-1-carboxylate [(XI), R1=2-cyano-5-(methylcarbamoyl)phenyl, R2=tert-butyl piperidine-1-carboxylate, R9=

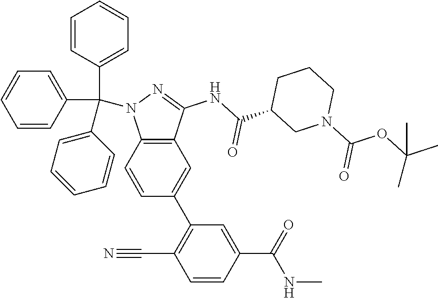 [SMILES: CNC(=O)c1ccc(C#N)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-{[5-(4,4,5,5-tetramethyl-1,3,2-dioxaborolan-2-yl)-1-trityl-1H-indazol-3-yl]carbamoyl}-piperidine-1-carboxylate and 3-bromo-4-cyano-N-methyl-benzamide. Flash column chromatography (Hexane/EtOAc 50:50). Obtained 0.035 g (56% of yield). LCMS (ESI) m/z 745 (M+H)⁺.

### tert-Butyl (3R)-3-[(5-{2-chloro-5-[(propan-2-yloxy)carbonyl]phenyl}-1-trityl-1H-indazol-3-yl)carbamoyl]-piperidine-1-carboxylate [(XI), R1=2-chloro-5-[(propan-2-yloxy)carbonyl]phenyl, R2=tert-butyl piperidine-1-carboxylate, R9=1,1′,1″-methanetriyltribenzene]

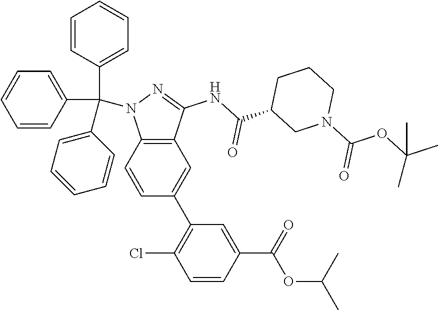 [SMILES: CC(C)OC(=O)c1ccc(Cl)c(-c2ccc3c(c2)c(NC(=O)[C@@H]2CCCN(C(=O)OC(C)(C)C)C2)nn3C(c2ccccc2)(c2ccccc2)c2ccccc2)c1]

Starting from tert-butyl (3R)-3-{[5-(4,4,5,5-tetramethyl-1,3,2-dioxaborolan-2-yl)-1-trityl-1H-indazol-3-yl]carbamoyl}-piperidine-1-carboxylate and propan-2-yl 3-bromo-4-chlorobenzoate. Flash column chromatography (Hexane/EtOAc 75:25). Obtained 0.050 g (77% of yield). ¹H NMR (500 MHz, DMSO-d₆) δ ppm 1.30 (d, J=6.25 Hz, 6H), 1.33-1.34 (m, 10H), 1.56-1.72 (m, 2H), 1.90-1.97 (m, 1H), 2.59 (m, 1H), 2.76-2.85 (m, 1H), 3.77 (d, J=13.12 Hz, 1H), 3.88-3.98 (m, 2H), 5.13 (quin, J=6.25 Hz, 1H), 6.47 (d, J=9.00 Hz, 1H), 7.15 (d, J=9.30 Hz, 1H), 7.24 (d, J=7.47 Hz, 6H), 7.28-7.31 (m, 3H), 7.34-7.37 (m, 6H), 7.70 (d, J=8.39 Hz, 1H), 7.80-7.73 (m, 2H), 7.90 (dd, J=8.24, 2.14 Hz, 1H), 10.65 (s, 1H). HRMS (ESI) calcd for C47H48ClN4O5 [M+H]⁺ 783.3308, found 783.3306. tert-Butyl (3R)-3-({5-[2,6-dichloro-3-(methoxycarbonyl)phenyl]-1-trityl-1H-indazol-3-yl)carbamoyl)piperidine-1-carboxylate [(XI), R1=2,6-dichloro-3-(methoxycarbonyl)phenyl, R2=tert-butyl piperidine-1-carboxylate, R9=1,1′,1″-methanetriyltribenzene]

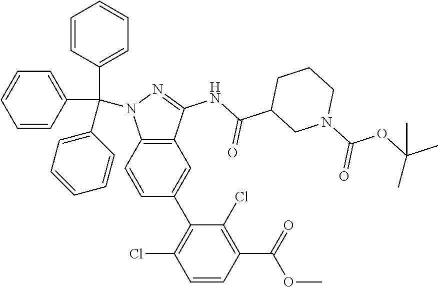 [SMILES: COC(=O)c1ccc(Cl)c(-c2ccc3c(c2)c(NC(=O)C2CCCN(C(=O)OC(C)(C)C)C2)nn3C(c2ccccc2)(c2ccccc2)c2ccccc2)c1Cl]

Starting from tert-butyl (3R)-3-{[5-(4,4,5,5-tetramethyl-1,3,2-dioxaborolan-2-yl)-1-trityl-1H-indazol-3-yl]carbamoyl}-piperidine-1-carboxylate and methyl 3-bromo-2,4-dichlorobenzoate. Flash column chromatography (Hexane/EtOAc 80:20). Obtained 0.056 g (72% of yield). ¹H NMR (500 MHz, DMSO-d₆) δ ppm 1.29-1.35 (m, 10H), 1.56-1.74 (m, 2H), 1.90-1.93 (m, 1H), 2.57 (m, 1H), 2.73-2.80 (m, 1H), 3.74 (d, J=14.03 Hz, 1H), 3.86 (s, 3H), 3.91-3.97 (m, 2H), 6.48 (d, J=9.00 Hz, 1H), 6.94 (d, J=10.07 Hz, 1H), 7.25-7.30 (m, 9H), 7.33-7.36 (m, 6H), 7.65-7.70 (m, 2H), 7.75 (d, J=8.39 Hz, 1H), 10.63 (s, 1H). HRMS (ESI) calcd for C45H43Cl2N4O5 [M+H]⁺ 789.2605, found 789.2589.

### tert-Butyl (3R)-3-({5-[2-chloro-5-(1,2,4-oxadiazol-3-yl)phenyl]-1-trityl-1H-indazol-3-yl}carbamoyl)piperidine-1-carboxylate [(XI), R1=2-chloro-5-(1,2,4-oxadiazol-3-yl)phenyl, R2=tert-butyl piperidine-1-carboxylate, R9=1,1′,1″-methanetriyltribenzene]

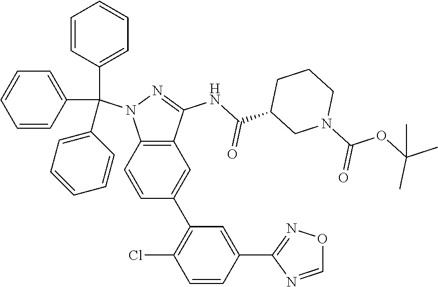 [SMILES: CC(C)(C)OC(=O)N1CCC[C@@H](C(=O)Nc2nn(C(c3ccccc3)(c3ccccc3)c3ccccc3)c3ccc(-c4cc(-c5ncon5)ccc4Cl)cc23)C1]

Starting from tert-butyl (3R)-3-{[5-(4,4,5,5-tetramethyl-1,3,2-dioxaborolan-2-yl)-1-trityl-1H-indazol-3-yl]carbamoyl}-piperidine-1-carboxylate and 3-(4-chloro-3-iodophenyl)-1,2,4-oxadiazole. Flash column chromatography (Hexane/EtOAc 70:30). Obtained 0.023 g (39% of yield). LCMS (ESI) m/z 765 (M+H)⁺.

### tert-Butyl (3R)-3-({5-[2-chloro-5-(1,3,4-oxadiazol-2-yl)phenyl]-1-trityl-1H-indazol-3-yl}carbamoyl)piperidine-1-carboxylate [(XI), R1=2-chloro-5-(1,3,4-oxadiazol-2-yl)phenyl, R2=tert-butyl piperidine-1-carboxylate, R9=1,1′,1″-methanetriyltribenzene]

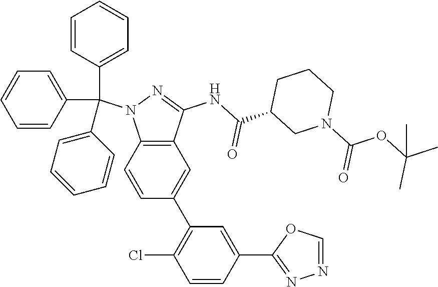 [SMILES: CC(C)(C)OC(=O)N1CCC[C@@H](C(=O)Nc2nn(C(c3ccccc3)(c3ccccc3)c3ccccc3)c3ccc(-c4cc(-c5nnco5)ccc4Cl)cc23)C1]

Starting from tert-butyl (3R)-3-{[5-(4,4,5,5-tetramethyl-1,3,2-dioxaborolan-2-yl)-1-trityl-1H-indazol-3-yl]carbamoyl}-piperidine-1-carboxylate and 2-(3-bromo-4-chlorophenyl)-1,3,4-oxadiazole. Flash column chromatography (Hexane/EtOAc 60:40). Obtained 0.061 g (95% of yield). ¹H NMR (500 MHz, DMSO-d₆) δ ppm 1.31-1.37 (m, 10H), 1.55-1.75 (m, 2H), 1.92-1.95 (m, 1H), 2.59 (m, 1H), 2.74-2.85 (m, 1H), 3.77 (d, J=13.12 Hz, 1H), 3.90-3.99 (m, 2H), 6.49 (d, J=8.85 Hz, 1H), 7.20 (d, J=9.30 Hz, 1H), 7.24 (d, J=7.63 Hz, 6H), 7.28-7.31 (m, 3H), 7.34-7.37 (m, 6H), 7.80 (d, J=8.39 Hz, 1H), 7.89-7.92 (m, 2H), 8.00 (dd, J=8.31, 2.21 Hz, 1H), 9.38 (s, 1H), 10.66 (s, 1H). HRMS (ESI) calcd for C45H42ClN6O4Na [M+H+Na]⁺787.2770, found 787.2787.

### tert-butyl (3R)-3-({5-[2-chloro-5-(5-methyl-1,3,4-oxadiazol-2-yl)phenyl]-1-trityl-1H-indazol-3-yl}carbamoyl)-piperidine-1-carboxylate [(XI), R1=2-chloro-5-(5-methyl-1,3,4-oxadiazol-2-yl)phenyl, R2=tert-butyl piperidine-1-carboxylate, R9=1,1′,1″-methanetriyltribenzene]

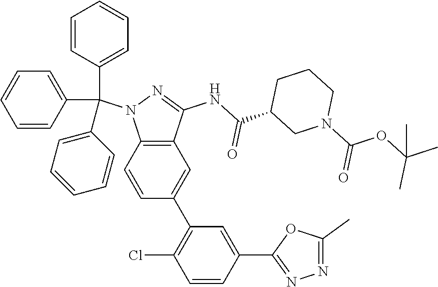 [SMILES: Cc1nnc(-c2ccc(Cl)c(-c3ccc4c(c3)c(NC(=O)[C@@H]3CCCN(C(=O)OC(C)(C)C)C3)nn4C(c3ccccc3)(c3ccccc3)c3ccccc3)c2)o1]

Starting from tert-butyl (3R)-3-{[5-(4,4,5,5-tetramethyl-1,3,2-dioxaborolan-2-yl)-1-trityl-1H-indazol-3-yl]carbamoyl}-piperidine-1-carboxylate and 2-(3-bromo-4-chlorophenyl)-5-methyl-1,3,4-oxadiazole. Flash column chromatography (Hexane/EtOAc 50:50). Obtained 0.066 g (99% of yield). 1H NMR (500 MHz, DMSO-d₆) δ ppm 1.30-1.37 (m, 10H), 1.59-1.70 (m, 2H), 1.91-1.96 (m, 1H), 2.57-2.60 (m, 4H), 2.74-2.83 (m, 1H), 3.77 (d, J=12.05 Hz, 1H), 3.86-3.98 (m, 2H), 6.49 (d, J=8.85 Hz, 1H), 7.18 (d, J=9.00 Hz, 1H), 7.24 (d, J=7.47 Hz, 6H), 7.28-7.31 (m, 3H), 7.34-7.37 (m, 6H), 7.78 (d, J=8.39 Hz, 1H), 7.84 (d, J=1.83 Hz, 1H), 7.89 (s, 1H), 7.95 (dd, J=8.39, 2.14 Hz, 1H), 10.66 (s, 1H). HRMS (ESI) calcd for C46H44ClN6O4 [M+H]⁺ 779.3107, found 779.3118.

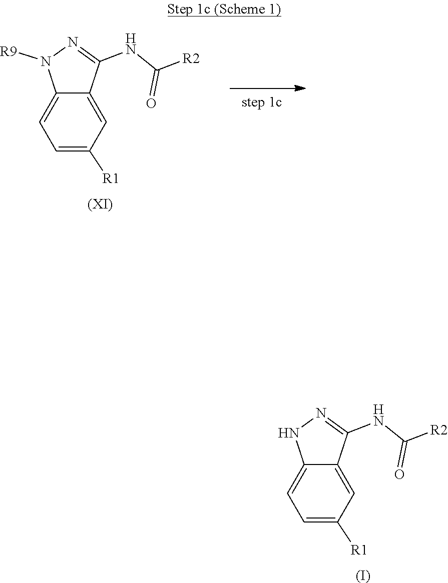

### N-[5-(4-Amino-2-chlorophenyl)-1H-indazol-3-yl]piperidine-3-carboxamide dihydrochloride (cpd 1) [(I), R1=4-amino-2-chlorophenyl, R2=piperidine, R3=H]

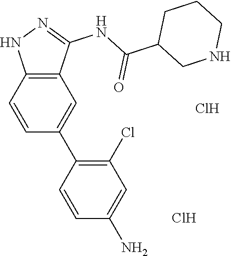 [SMILES: Nc1ccc(-c2ccc3[nH]nc(NC(=O)C4CCCNC4)c3c2)c(Cl)c1 ; Cl ; Cl]

To a solution of tert-butyl 3-{[5-(4-amino-2-chlorophenyl)-1-trityl-1H-indazol-3-yl]carbamoyl}piperidine-1-carboxylate (0.082 g, 0.115 mmol) in dry DCM (5 mL), triethylsilane (0,055 mL, 0.345 mmol) and 4M HCl in dioxane (0.29 mL, 1.15 mmol) were added. The mixture was stirred at room temperature for 3 h, then the resulting suspension was evaporated to dryness. The crude was treated with MTBE (10 mL), stirred for 10 min. and filtered. After drying at 40° C. under vacuo N-[5-(4-amino-2-chlorophenyl)-1H-indazol-3-yl]piperidine-3-carboxamide dihydrochloride (0.041 g, 87% of yield) was obtained as white solid. 1H NMR (500 MHz, DMSO-d₆) δ ppm 1.67-1.76 (m, 2H), 1.81-1.85 (m, 2H), 2.09 (m, 1H), 2.87-3.15 (m, 3H), 6.82 (d, J=7.47 Hz, 1H), 6.95 (br. s., 1H), 7.17 (d, J=8.24 Hz, 1H), 7.34 (dd, J=8.62, 1.30 Hz, 1H), 7.47 (d, J=8.69 Hz, 1H), 7.70 (s, 1H), 8.70 (br. s., 1H), 8.79 (d, J=8.69 Hz, 1H), 10.67 (s, 1H), 12.80 (br. s., 1H). HRMS (ESI) calcd for C19H21ClN5O [M+H]⁺ 370.1429, found 370.1438.

According to this same methodology, but employing suitable intermediates and in some cases trifluoroacetic acid or p-toluenesulfonic acid instead of 4M HCl in dioxane, the following compounds were prepared:

### N-[5-(2,4-Dichlorophenyl)-1H-indazol-3-yl]-1-methylpiperidine-3-carboxamide hydrochloride (cpd 2) [(I), R1=2,4-dichlorophenyl, R2=1-methylpiperidine, R3=H]

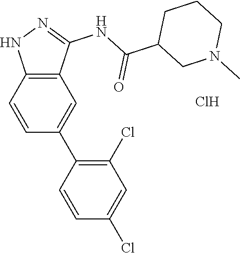 [SMILES: CN1CCCC(C(=O)Nc2n[nH]c3ccc(-c4ccc(Cl)cc4Cl)cc23)C1 ; Cl]

Obtained 0.007 g (70% of yield). ¹H NMR (500 MHz, DMSO-d₆) δ ppm 1.49-1.59 (m, 1H), 1.73-1.95 (m, 2H), 2.10-2.16 (m, 1H), 2.76 (d, J=4.73 Hz, 3H), 2.86-2.94 (m, 1H), 3.02-3.27 (m, 2H), 3.53-3.59 (m, 1H), 7.39 (dd, J=8.54, 1.53 Hz, 1H) 7.44 (d, J=8.24 Hz, 1H), 7.51-7.54 (m, 2H), 7.74 (d, J=2.14 Hz, 1H), 7.68-7.76 (m, 1H), 10.01 (br. s., 1H), 10.74 (br. s., 1H), 12.89 (br. s., 1H). HRMS (ESI) calcd for C20H21Cl2N4O [M+H]⁺ 403.1087, found 403.1095.

### N-[5-(2-Chloro-4-hydroxyphenyl)-1H-indazol-3-yl]piperidine-3-carboxamide hydrochloride (cpd 3) [(I), R1=2-chloro-4-hydroxyphenyl, R2=piperidine, R3=H]

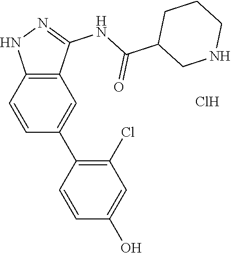 [SMILES: O=C(Nc1n[nH]c2ccc(-c3ccc(O)cc3Cl)cc12)C1CCCNC1 ; Cl]

Obtained 0.011 g (37% of yield). ¹H NMR (500 MHz, DMSO-d₆) δ ppm 1.63-1.90 (m, 3H), 2.08 (d, J=7.02 Hz, 1H), 2.87-3.21 (m, 4H), 3.29-3.37 (m, 2H), 6.83 (dd, J=8.39, 2.44 Hz, 1H), 6.94 (d, J=2.44 Hz, 1H), 7.21 (d, J=8.24 Hz, 1H), 7.34 (dd, J=8.77, 1.30 Hz, 1H), 7.47 (d, J=8.69 Hz, 1H), 7.71 (s, 1H), 8.52-8.76 (m, 2H), 9.99 (br. s., 1H), 10.67 (s, 1H), 12.80 (br. s., 1H). HRMS (ESI) calcd for C19H2OClN4O2 [M+H]⁺ 371.1270, found 371.1278.

### N-[5-(2,5-Dichlorophenyl)-1H-indazol-3-yl]piperidine-3-carboxamide hydrochloride (cpd 4) [(I), R1=2,5-dichlorophenyl, R2=piperidine, R3=H]

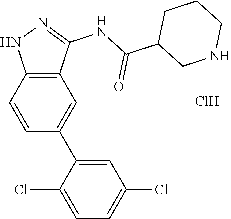 [SMILES: O=C(Nc1n[nH]c2ccc(-c3cc(Cl)ccc3Cl)cc12)C1CCCNC1 ; Cl]

Obtained 0.048 g (95% of yield). ¹H NMR (500 MHz, DMSO-d₆) δ ppm 1.62-1.88 (m, 3H), 2.04-2.16 (m, 1H), 2.83-3.01 (m, 2H), 3.04-3.20 (m, 3H), 7.41 (dd, J=8.69, 1.53 Hz, 1H), 7.46-7.51 (m, 2H), 7.54 (d, J=8.54 Hz, 1 H), 7.61 (dd, J=7.32, 1.22 Hz, 1H), 7.83 (s, 1H), 8.52-8.76 (m, 2H), 10.73 (s, 1H), 12.90 (br. s., 1H). HRMS (ESI) calcd for C19H19Cl2N4O [M+H]⁺ 389.0931, found 389.0929.

### (3R)-N-[5-(4-Amino-2-chlorophenyl)-1H-indazol-3-yl]piperidine-3-carboxamide dihydrochloride (cpd 5) [(I), R1=4-amino-2-chlorophenyl, R2=piperidine, R3=H]

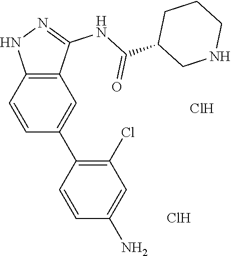 [SMILES: Nc1ccc(-c2ccc3[nH]nc(NC(=O)[C@@H]4CCCNC4)c3c2)c(Cl)c1 ; Cl ; Cl]

Obtained 0.029 g (83% of yield). ¹H NMR (500 MHz, DMSO-d₆) δ ppm 1.63-1.90 (m, 3H), 2.05-2.11 (m, 1H), 2.88-2.93 (m, 1H), 2.95-3.04 (m, 1H), 3.05-3.13 (m, 1H), 3.16 (d, J=11.59 Hz, 1H), 6.81 (d, J=7.93 Hz, 1H), 6.95 (br. s., 1H), 7.17 (d, J=8.24 Hz, 1H), 7.34 (dd, J=8.62, 1.45 Hz, 1H), 7.45-7.51 (m, 1H), 7.70 (s, 1H), 8.69 (br. s., 1H), 8.78 (d, J=9.76 Hz, 1H), 10.67 (s, 1H), 12.79 (br. s., 1H). HRMS (ESI) calcd for C19H21ClN5O [M+H]⁺370.1429, found 370.1436.

### (3R)-N-[5-(2-chloro-4-hydroxyphenyl)-1H-indazol-3-yl]piperidine-3-carboxamide hydrochloride (cpd 6) [(I), R1=2-chloro-4-hydroxyphenyl, R2=piperidine, R3=H]

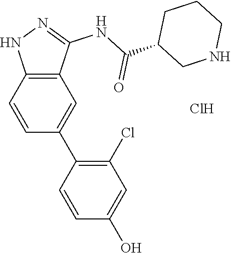 [SMILES: O=C(Nc1n[nH]c2ccc(-c3ccc(O)cc3Cl)cc12)[C@@H]1CCCNC1 ; Cl]

Obtained 0.030 g (97% of yield). 1H NMR (500 MHz, DMSO-d₆) δ ppm 1.63-1.90 (m, 3H), 2.08 (d, J=7.02 Hz, 1H), 2.87-3.21 (m, 4H), 3.29-3.37 (m, 2H), 6.83 (dd, J=8.39, 2.44 Hz, 1H), 6.94 (d, J=2.44 Hz, 1H), 7.21 (d, J=8.24 Hz, 1H), 7.34 (dd, J=8.77, 1.30 Hz, 1H), 7.47 (d, J=8.69 Hz, 1H), 7.71 (s, 1H), 8.52-8.76 (m, 2H), 9.99 (br. s., 1H), 10.67 (s, 1H), 12.80 (br. s., 1H). HRMS (ESI) calcd for C19H2OClN4O2 [M+H]⁺ 371.1270, found 371.1276.

### (3S)-N-[5-(4-Amino-2-chlorophenyl)-1H-indazol-3-yl]piperidine-3-carboxamide dihydrochloride (cpd 7) [(I), R1=4-amino-2-chlorophenyl, R2=piperidine, R3=H]

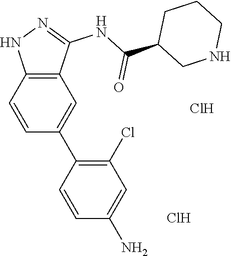 [SMILES: Nc1ccc(-c2ccc3[nH]nc(NC(=O)[C@H]4CCCNC4)c3c2)c(Cl)c1 ; Cl ; Cl]

Obtained 0.026 g (76% of yield). 1H NMR (500 MHz, DMSO-d₆) δ ppm 1.63-1.90 (m, 3H), 2.05-2.11 (m, 1H), 2.88-2.93 (m, 1H), 2.95-3.04 (m, 1H), 3.05-3.13 (m, 1H), 3.16 (d, J=11.59 Hz, 1H), 6.81 (d, J=7.93 Hz, 1H), 6.95 (br. s., 1H), 7.17 (d, J=8.24 Hz, 1H), 7.34 (dd, J=8.62, 1.45 Hz, 1H), 7.45-7.51 (m, 1H), 7.70 (s, 1H), 8.69 (br. s., 1H), 8.78 (d, J=9.76 Hz, 1H), 10.67 (s, 1H), 12.79 (br. s., 1H). HRMS (ESI) calcd for C19H21ClN5O [M+H]⁺370.1429, found 370.1431.

### (3R)-N-[5-(2-Chloro-4-methylphenyl)-1H-indazol-3-yl]piperidine-3-carboxamide hydrochloride (cpd 8) [(I), R1=2-chloro-4-methylphenyl, R2=piperidine, R3=H]

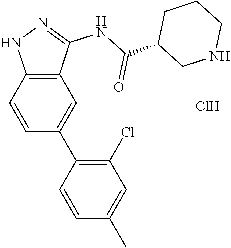 [SMILES: Cc1ccc(-c2ccc3[nH]nc(NC(=O)[C@@H]4CCCNC4)c3c2)c(Cl)c1 ; Cl]

Obtained 0.043 g (99% of yield). ¹H NMR (500 MHz, DMSO-d₆) δ ppm 1.65-1.88 (m, 3H), 2.06-2.12 (m, 1H), 2.36 (s, 3H), 2.92-3.02 (m, 2H), 3.06-3.12 (m, 1H), 3.16 (d, J=12.05 Hz, 1H), 3.31-3.36 (m, 1H), 7.23 (d, J=7.78 Hz, 1H), 7.28 (d, J=7.78 Hz, 1H), 7.37 (dd, J=8.69, 1.53 Hz, 1H), 7.40 (s, 1H), 7.51 (d, J=8.69 Hz, 1H), 7.76 (s, 1H), 8.54-8.84 (m, 2H), 10.70 (s, 1H), 12.84 (br. s., 1H). HRMS (ESI) calcd for C20H22ClN4O [M+H]⁺ 369.1477, found 369.1484.

### N-[5-(5-Carbamoyl-2-chlorophenyl)-1H-indazol-3-yl]piperidine-3-carboxamide hydrochloride (cpd 9) [(I), R1=5-carbamoyl-2-chlorophenyl, R2=piperidine, R3=H]

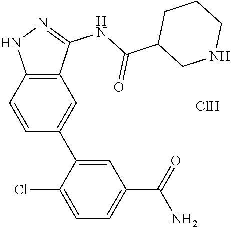 [SMILES: NC(=O)c1ccc(Cl)c(-c2ccc3[nH]nc(NC(=O)C4CCCNC4)c3c2)c1 ; Cl]

Obtained 0.025 g (96% of yield). ¹H NMR (500 MHz, DMSO-d₆) δ ppm 1.70-1.94 (m, 3H), 2.10-2.19 (m, 1H), 2.95 (d, J=11.74 Hz, 1H), 3.01 (m, 1H), 3.07-3.15 (m, 1H), 3.19 (d, J=9.91 Hz, 1H), 3.34-3.38 (m, 1H), 7.46 (dd, J=8.69, 1.53 Hz, 1H), 7.54 (s, 1H), 7.58 (d, J=8.69 Hz, 1H), 7.69 (d, J=8.24 Hz, 1H), 7.87 (s, 1H), 7.91 (dd, J=8.31, 2.21 Hz, 1H), 7.95 (d, J=2.14 Hz, 1H), 8.16 (s, 1H), 8.62-8.86 (m, 2H), 10.76 (s, 1H), 12.93 (br. s., 1H). HRMS (ESI) calcd for C20H21ClN5O2 [M+H]⁺ 398.1379, found 398.1387.

### (3S)-N-[5-(2-chloro-4-hydroxyphenyl)-1H-indazol-3-yl]piperidine-3-carboxamide hydrochloride (cpd 10) [(I), R1=2-chloro-4-hydroxyphenyl, R2=piperidine, R3=H]

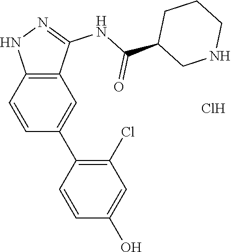 [SMILES: O=C(Nc1n[nH]c2ccc(-c3ccc(O)cc3Cl)cc12)[C@H]1CCCNC1 ; Cl]

Obtained 0.027 g (74% of yield). 1H NMR (500 MHz, DMSO-d₆) δ ppm 1.63-1.90 (m, 3H), 2.08 (d, J=7.02 Hz, 1H), 2.87-3.21 (m, 4H), 3.29-3.37 (m, 2H), 6.83 (dd, J=8.39, 2.44 Hz, 1H), 6.94 (d, J=2.44 Hz, 1H), 7.21 (d, J=8.24 Hz, 1H), 7.34 (dd, J=8.77, 1.30 Hz, 1H), 7.47 (d, J=8.69 Hz, 1H), 7.71 (s, 1H), 8.52-8.76 (m, 2H), 9.99 (br. s., 1H), 10.67 (s, 1H), 12.80 (br. s., 1H). HRMS (ESI) calcd for C19H2OClN4O2 [M+H]⁺ 371.1270, found 371.1272.

### N-[5-(3-Amino-2-fluorophenyl)-1H-indazol-3-yl]piperidine-3-carboxamide hydrochloride (cpd 11) [(I), R1=3-amino-2-fluorophenyl, R2=piperidine, R3=H]

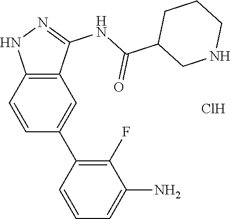 [SMILES: Nc1cccc(-c2ccc3[nH]nc(NC(=O)C4CCCNC4)c3c2)c1F ; Cl]

Obtained 0.036 g (99% of yield). 1H NMR (500 MHz, DMSO-d₆) δ ppm 1.63-1.95 (m, 3H), 2.07-2.17 (m, 1H), 2.92 (d, J=9.30 Hz, 1H), 3.00 (m, 1H), 3.08 (m, 1H), 3.18 (d, J=12.81 Hz, 2H), 6.73 (m, 1H), 6.88 (m, 1H), 7.01 (m, 1H), 7.45 (d, J=8.69 Hz, 1H), 7.51 (d, J=8.69 Hz, 1H), 7.89 (s, 1H), 8.61-8.90 (m, 2H), 10.71 (s, 1H), 12.83 (br. s., 1H). HRMS (ESI) calcd for C19H21FN5O [M+H]⁺ 354.1725, found 354.1730.

### (3S)-N-[5-(2-Chloro-4-methylphenyl)-1H-indazol-3-yl]piperidine-3-carboxamide hydrochloride (cpd 12) [(I), R1=2-chloro-4-methylphenyl, R2=piperidine, R3=H]

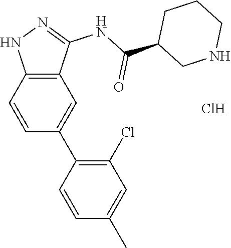 [SMILES: Cc1ccc(-c2ccc3[nH]nc(NC(=O)[C@H]4CCCNC4)c3c2)c(Cl)c1 ; Cl]

Obtained 0.037 g (98% of yield). ¹H NMR (500 MHz, DMSO-d₆) δ ppm 1.65-1.88 (m, 3H), 2.06-2.12 (m, 1H), 2.36 (s, 3H), 2.92-3.02 (m, 2H), 3.06-3.12 (m, 1H), 3.16 (d, J=12.05 Hz, 1H), 3.31-3.36 (m, 1H), 7.23 (d, J=7.78 Hz, 1H), 7.28 (d, J=7.78 Hz, 1H), 7.37 (dd, J=8.69, 1.53 Hz, 1H), 7.40 (s, 1H), 7.51 (d, J=8.69 Hz, 1H), 7.76 (s, 1H), 8.54-8.84 (m, 2H), 10.70 (s, 1H), 12.84 (br. s., 1H). HRMS (ESI) calcd for C20H22ClN4O [M+H]⁺ 369.1477, found 369.1484.

### (3R)-N-[5-(2,5-Dichlorophenyl)-1H-indazol-3-yl]piperidine-3-carboxamide hydrochloride (cpd 13) [(I), R1=2,5-dichlorophenyl, R2=piperidine, R3=H]

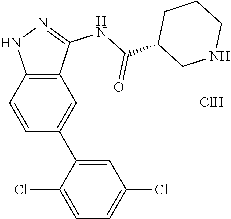 [SMILES: O=C(Nc1n[nH]c2ccc(-c3cc(Cl)ccc3Cl)cc12)[C@@H]1CCCNC1 ; Cl]

Obtained 0.016 g (47% of yield). ¹H NMR (500 MHz, DMSO-d₆) δ ppm 1.62-1.88 (m, 3H), 2.04-2.16 (m, 1H), 2.83-3.01 (m, 2H), 3.04-3.20 (m, 3H), 7.41 (dd, J=8.69, 1.53 Hz, 1H), 7.46-7.51 (m, 2H), 7.54 (d, J=8.54 Hz, 1H), 7.61 (dd, J=7.32, 1.22 Hz, 1H), 7.83 (s, 1H), 8.52-8.76 (m, 2H), 10.73 (s, 1H), 12.90 (br. s., 1H). HRMS (ESI) calcd for C19H19Cl2N4O [M+H]⁺389.0931, found 389.0937.

### (3R)-N-[5-(2,4-Dichlorophenyl)-1H-indazol-3-yl]piperidine-3-carboxamide hydrochloride (cpd 14) [(I), R1=2,4-dichlorophenyl, R2=piperidine, R3=H]

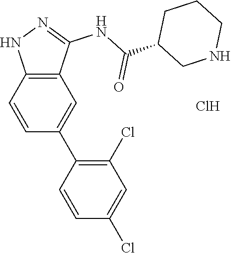 [SMILES: O=C(Nc1n[nH]c2ccc(-c3ccc(Cl)cc3Cl)cc12)[C@@H]1CCCNC1 ; Cl]

Obtained 0.027 g (93% of yield). 1H NMR (500 MHz, DMSO-d₆) δ ppm 1.60-1.77 (m, 3H), 1.83 (br. s., 1H), 2.04-2.14 (m, 1H), 2.83-3.03 (m, 2H), 3.04-3.13 (m, 1H), 3.17 (d, J=12.51 Hz, 1H), 7.39 (dd, J=8.69, 1.53 Hz, 1H), 7.44 (d, J=8.39 Hz, 1H), 7.51-7.55 (m, 2H), 7.74 (d, J=2.14 Hz, 1H), 7.80 (s, 1H), 8.58-8.77 (m, 2H), 10.73 (s, 1H), 12.89 (br. s., 1H).). HRMS (ESI) calcd for C19H19Cl2N4O [M+H]⁺ 389.0931, found 389.0941.

### N-{5-[5-Chloro-2-(trifluoromethyl)phenyl]-1H-indazol-3-yl}piperidine-3-carboxamide trifluoroacetate (cpd 15) [(I), R1=5-chloro-2-(trifluoromethyl)phenyl, R2=piperidine, R3=H]

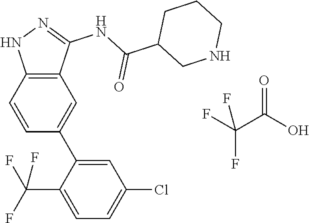

Obtained 0.030 g (88% of yield). ¹H NMR (500 MHz, DMSO-d₆) δ ppm 1.69-1.72 (m, 3H) 1.77-1.86 (m, 1H), 2.02-2.12 (m, 1H), 2.93 (m, 1H), 3.07-3.18 (m, 1H), 3.33-3.36 (m, 2H), 7.31 (d, J=8.85 Hz, 1H), 7.46-7.55 (m, 2H), 7.71 (dd, J=8.54, 1.53 Hz, 1H), 7.76 (s, 1H), 7.87 (d, J=8.54 Hz, 1H), 8.48 (br. s., 2H), 10.72 (s, 1H), 12.90 (br. s., 1H). HRMS (ESI) calcd for C20H19ClF3N4O [M+H]⁺ 423.1194, found 423.1190.

### N-[5-(5-Acetyl-2-fluorophenyl)-1H-indazol-3-yl]piperidine-3-carboxamide hydrochloride (cpd 16) [(I), R1=5-acetyl-2-fluorophenyl, R2=piperidine, R3=H]

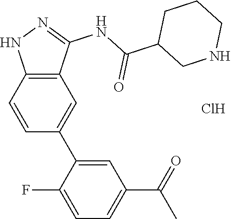 [SMILES: CC(=O)c1ccc(F)c(-c2ccc3[nH]nc(NC(=O)C4CCCNC4)c3c2)c1 ; Cl]

Obtained 0.070 g (95% of yield). ¹H NMR (500 MHz, DMSO-d₆) δ ppm 1.69-1.85 (m, 3H), 2.11 (m, 1H), 2.64 (s, 3H), 2.90-3.03 (m, 2H), 3.06-3.22 (m, 1H), 3.35-3.38 (m, 2H), 7.44-7.66 (m, 3H), 7.94-8.14 (m, 3H), 8.56-8.84 (m, 2H), 10.75 (s, 1H), 12.91 (br. s., 1H). HRMS (ESI) calcd for C21H22FN4O2 [M+H]⁺ 381.1722, found 381.1718.

### N-[5-(2-Chloro-5-methoxyphenyl)-1H-indazol-3-yl]piperidine-3-carboxamide hydrochloride (cpd 17) [(I), R1=2-chloro-5-methoxyphenyl, R2=piperidine, R3=H]

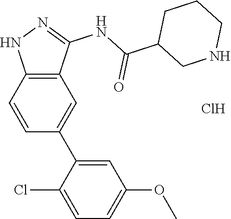 [SMILES: COc1ccc(Cl)c(-c2ccc3[nH]nc(NC(=O)C4CCCNC4)c3c2)c1 ; Cl]

Obtained 0.030 g (91% of yield). ¹H NMR (500 MHz, DMSO-d₆) δ ppm 1.66-1.90 (m, 3H), 2.05-2.15 (m, 1H), 2.86-3.02 (m, 2H), 3.04-3.21 (m, 2H), 3.30-3.38 (m, 1H), 3.79 (s, 3H), 6.94 (d, J=3.05 Hz, 1H), 6.99 (dd, J=8.77, 3.13 Hz, 1H), 7.40 (dd, J=8.69, 1.53 Hz, 1H), 7.47-7.49 (m, 1H), 7.52 (d, J=8.69 Hz, 1H), 7.80 (s, 1H), 8.55-8.81 (m, 2H), 10.71 (s, 1H), 12.86 (br. s., 1H). HRMS (ESI) calcd for C20H22ClN4O2 [M+H]⁺ 385.1426, found 385.1432.

### (3R)-N-[5-(2-Chloro-5-methoxyphenyl)-1H-indazol-3-yl]piperidine-3-carboxamide hydrochloride (cpd 18) [(I), R1=2-chloro-5-methoxyphenyl, R2=piperidine, R3=H]

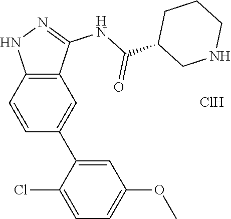 [SMILES: COc1ccc(Cl)c(-c2ccc3[nH]nc(NC(=O)[C@@H]4CCCNC4)c3c2)c1 ; Cl]

Obtained 0.035 g (94% of yield). ¹H NMR (500 MHz, DMSO-d₆) δ ppm 1.66-1.90 (m, 3H), 2.05-2.15 (m, 1H), 2.86-3.02 (m, 2H), 3.04-3.21 (m, 2H), 3.30-3.38 (m, 1H), 3.79 (s, 3H), 6.94 (d, J=3.05 Hz, 1H), 6.99 (dd, J=8.77, 3.13 Hz, 1H), 7.40 (dd, J=8.69, 1.53 Hz, 1H), 7.47-7.49 (m, 1H), 7.52 (d, J=8.69 Hz, 1H), 7.80 (s, 1H), 8.55-8.81 (m, 2H), 10.71 (s, 1H), 12.86 (br. s., 1H). HRMS (ESI) calcd for C20H22ClN4O2 [M+H]⁺ 385.1426, found 385.1430.

### (3R)-N-[5-(5-Acetyl-2-fluorophenyl)-1H-indazol-3-yl]piperidine-3-carboxamide hydrochloride (cpd 19) [(I), R1=5-acetyl-2-fluorophenyl, R2=piperidine, R3=H]

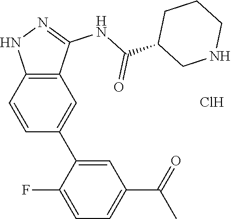 [SMILES: CC(=O)c1ccc(F)c(-c2ccc3[nH]nc(NC(=O)[C@@H]4CCCNC4)c3c2)c1 ; Cl]

Obtained 0.033 g (99% of yield). ¹H NMR (500 MHz, DMSO-d₆) δ ppm 1.69-1.85 (m, 3H), 2.11 (m, 1H), 2.64 (s, 3H), 2.90-3.03 (m, 2H), 3.06-3.22 (m, 1H), 3.35-3.38 (m, 2H), 7.44-7.66 (m, 3H), 7.94-8.14 (m, 3H), 8.56-8.84 (m, 2H), 10.75 (s, 1H), 12.91 (br. s., 1H). HRMS (ESI) calcd for C21H22FN4O2 [M+H]⁺ 381.1722, found 381.1724.

### (3R)-N-[5-(2-Fluoro-5-methoxyphenyl)-1H-indazol-3-yl]piperidine-3-carboxamide hydrochloride (cpd 20) [(I), R1=2-fluoro-5-methoxyphenyl, R2=piperidine, R3=H]

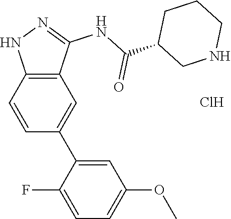 [SMILES: COc1ccc(F)c(-c2ccc3[nH]nc(NC(=O)[C@@H]4CCCNC4)c3c2)c1 ; Cl]

Obtained 0.029 g (90% of yield). ¹H NMR (500 MHz, DMSO-d₆) δ ppm 1.64-1.92 (m, 3H), 2.09 (m, 1H), 2.87-3.03 (m, 2H), 3.05-3.24 (m, 2H), 3.79 (s, 3H), 6.95 (dt, J=8.85, 3.43 Hz, 1H), 6.99 (dd, J=6.41, 3.05 Hz, 1H), 7.24 (t, J=6.41, 9.30 Hz, 1H), 7.50-7.57 (m, 2H), 7.92 (s, 1H), 8.46-8.89 (m, 2H), 10.71 (s, 1H), 12.86 (br. s., 1H). HRMS (ESI) calcd for C20H22FN4O2 [M+H]⁺ 369.1722, found 369.1725.

### (3R)-N-[5-(5-Chloro-2-fluorophenyl)-1H-indazol-3-yl]piperidine-3-carboxamide hydrochloride (cpd 21) [(I), R1=5-chloro-2-fluorophenyl, R2=piperidine, R3=H]

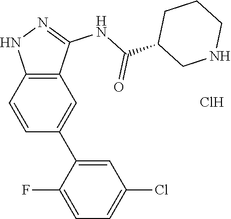 [SMILES: O=C(Nc1n[nH]c2ccc(-c3cc(Cl)ccc3F)cc12)[C@@H]1CCCNC1 ; Cl]

Obtained 0.030 g (87% of yield). ¹H NMR (500 MHz, DMSO-d₆) δ ppm 1.66-1.87 (m, 3H), 1.85 (br. s., 1H), 2.09 (m, 1H), 2.86-3.02 (m, 2H), 3.07-3.23 (m, 2H), 7.39 (m, 1H), 7.47 (ddd, J=6.56, 4.27, 2.14 Hz, 1H), 7.51-7.58 (m, 3H), 7.96 (s, 1H), 8.60-8.84 (m, 2H), 10.74 (s, 1H), 12.90 (br. s., 1H). HRMS (ESI) calcd for C19H19ClFN4O [M+H]⁺ 373.1226, found 373.1236.

### (3R)-N-[5-(5-Ethoxy-2-fluorophenyl)-1H-indazol-3-yl]piperidine-3-carboxamide hydrochloride (cpd 22) [(I), R1=5-ethoxy-2-fluorophenyl, R2=piperidine, R3=H]

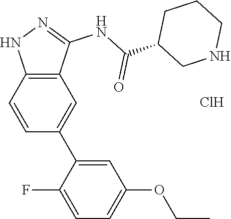 [SMILES: CCOc1ccc(F)c(-c2ccc3[nH]nc(NC(=O)[C@@H]4CCCNC4)c3c2)c1 ; Cl]

Obtained 0.028 g (84% of yield). ¹H NMR (500 MHz, DMSO-d₆) δ ppm 1.34 (t, J=7.02 Hz, 3H), 1.65-1.88 (m, 3H), 2.09 (m, 1H), 2.87-3.04 (m, 2H), 3.06-3.25 (m, 2H), 4.06 (q, J=7.02 Hz, 2H), 6.90-6.95 (m, 1H), 6.96-7.00 (m, 1H), 7.22 (m, 1H), 7.49-7.56 (m, 2H), 7.92 (s, 1H), 8.54-8.84 (m, 2H), 10.71 (s, 1H), 12.86 (br. s., 1H). HRMS (ESI) calcd for C21H24FN4O2 [M+H]⁺ 383.1878, found 383.1883.

### (3R)-N-{5-[2-Fluoro-5-(trifluoromethoxy)phenyl]-1H-indazol-3-yl}piperidine-3-carboxamide hydrochloride (cpd 23) [(I), R1=2-fluoro-5-(trifluoromethoxy)phenyl, R2=piperidine, R3=H]

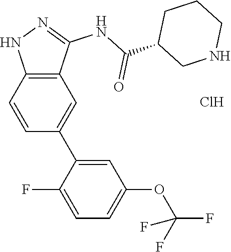 [SMILES: O=C(Nc1n[nH]c2ccc(-c3cc(OC(F)(F)F)ccc3F)cc12)[C@@H]1CCCNC1 ; Cl]

Obtained 0.029 g (87% of yield). ¹H NMR (500 MHz, DMSO-d₆) δ ppm 1.56-1.89 (m, 3H), 2.11 (m, 1H), 2.82-3.04 (m, 2H), 3.07-3.27 (m, 2H), 7.37-7.61 (m, 5H), 7.98 (s, 1H), 8.57-8.84 (m, 2H), 10.75 (s, 1H), 12.92 (br. s., 1H). HRMS (ESI) calcd for C20H19F4N4O2 [M+H]⁺ 423.1439, found 423.1443.

### (3R)-N-[5-(5-Cyano-2-methylphenyl)-1H-indazol-3-yl]piperidine-3-carboxamide hydrochloride (cpd 24) [(I), R1=5-cyano-2-methylphenyl, R2=piperidine, R3=H]

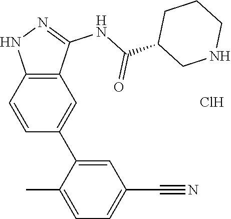 [SMILES: Cc1ccc(C#N)cc1-c1ccc2[nH]nc(NC(=O)[C@@H]3CCCNC3)c2c1 ; Cl]

Obtained 0.025 g (81% of yield). ¹H NMR (500 MHz, DMSO-d₆) δ ppm 1.63-1.89 (m, 3H), 2.02-2.14 (m, 1H), 2.30 (s, 3H), 2.83-3.02 (m, 2H), 3.05-3.18 (m, 2H), 7.36 (dd, J=8.54, 1.37 Hz, 1H), 7.51-7.57 (m, 2H), 7.65 (d, J=1.37 Hz, 1H), 7.72-7.77 (m, 2H), 8.56-8.76 (m, 2H), 10.71 (s, 1H), 12.87 (br. s., 1H). HRMS (ESI) calcd for C21H22N5O [M+H]⁺ 360.1819, found 360.1830.

### (3R)-N-[5-(5-Cyano-2-fluorophenyl)-1H-indazol-3-yl]piperidine-3-carboxamide hydrochloride (cpd 25) [(I), R1=5-cyano-2-fluorophenyl, R2=piperidine, R3=H]

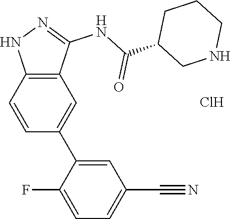 [SMILES: N#Cc1ccc(F)c(-c2ccc3[nH]nc(NC(=O)[C@@H]4CCCNC4)c3c2)c1 ; Cl]

Obtained 0.018 g (86% of yield). ¹H NMR (500 MHz, DMSO-d₆) δ ppm 1.65-1.92 (m, 3H), 2.11 (m, 1H), 2.84-3.03 (m, 2H), 3.09-3.21 (m, 2H), 7.53-7.62 (m, 3H), 7.94 (ddd, J=8.46, 4.50, 2.14 Hz, 1H), 7.99 (s, 1H), 8.05 (dd, J=7.24, 1.91 Hz, 1H), 8.61-8.80 (m, 2H), 10.75 (s, 1H), 12.94 (br. s., 1H). HRMS (ESI) calcd for C20H18FN5O [M+H]⁺ 364.1568, found 364.1568.

### (3R)-N-[5-(2-Chloro-5-nitrophenyl)-1H-indazol-3-yl]piperidine-3-carboxamide hydrochloride (cpd 26) [(I), R1=2-chloro-5-nitrophenyl, R2=piperidine, R3=H]

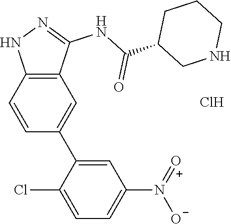

Obtained 0.010 g (31% of yield). ¹H NMR (500 MHz, DMSO-d₆) δ ppm 1.60-1.87 (m, 3H), 2.09 (m, 1H), 2.83-3.02 (m, 2H), 3.04-3.13 (m, 1H), 3.17 (d, J=12.35 Hz, 1H), 3.36 (m, 1H), 7.46 (dd, J=8.85, 1.53 Hz, 1H), 7.58 (d, J=8.69 Hz, 1H), 7.86-7.96 (m, 2H), 8.18 (d, J=2.75 Hz, 1H), 8.25 (dd, J=8.77, 2.82 Hz, 1H), 8.57-8.78 (m, 2H), 10.77 (s, 1H), 12.96 (br. s., 1H). HRMS (ESI) calcd for C19H19ClN5O3 [M+H]⁺ 400.1171, found 400.1174.

### N-[5-(2,6-Dichlorophenyl)-1H-indazol-3-yl]-6-oxopiperidine-3-carboxamide (cpd 27) [(I), R1=2,6-dichlorophenyl, R2=piperidin-2-one, R3=H]

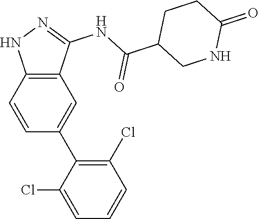 [SMILES: O=C1CCC(C(=O)Nc2n[nH]c3ccc(-c4c(Cl)cccc4Cl)cc23)CN1]

Obtained 0.021 g (62% of yield). ¹H NMR (500 MHz, DMSO-d₆) δ ppm 1.79-1.95 (m, 1H), 1.99-2.04 (m, 1H), 2.13-2.29 (m, 2H), 2.89 (m, 1H), 3.24-3.37 (m, 2H), 7.18 (dd, J=8.54, 1.37 Hz, 1H), 7.44 (m, 1H), 7.48-7.55 (m, 2H), 7.59 (d, J=8.08 Hz, 2H), 7.70 (s, 1H), 10.58 (s, 1H), 12.83 (br. s., 1H). HRMS (ESI) calcd for C19H17Cl2N4O2 [M+H]⁺ 403.0723, found 403.0723.

### N-[5-(5-Acetyl-2-fluorophenyl)-1H-indazol-3-yl]-6-oxopiperidine-3-carboxamide (cpd 28) [(I), R1=5-acetyl-2-fluorophenyl, R2=piperidin-2-one, R3=H]

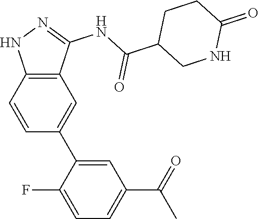 [SMILES: CC(=O)c1ccc(F)c(-c2ccc3[nH]nc(NC(=O)C4CCC(=O)NC4)c3c2)c1]

Obtained 0.046 g (99% of yield). ¹H NMR (500 MHz, DMSO-d₆) δ ppm 1.83-1.97 (m, 1H), 2.02-2.07 (m, 1H), 2.16-2.32 (m, 2H), 2.63 (s, 3H), 2.92 (dt, J=9.65, 4.86 Hz, 1H), 3.29-3.41 (m, 2H), 7.46 (m, 1H), 7.52-7.59 (m, 2H), 7.98 (s, 1H), 8.02 (ddd, J=8.50, 4.84, 2.21 Hz, 1H), 8.05 (dd, J=7.78, 1.83 Hz, 1H), 10.60 (s, 1H), 12.86 (br. s., 1H). HRMS (ESI) calcd for C21H20FN4O3 [M+H]⁺ 395.1514, found 395.1516.

### Methyl 4-chloro-3-(3-{[(3R)-piperidin-3-ylcarbonyl]amino}-1H-indazol-5-yl)benzoate hydrochloride (cpd 29) [(I), R1=methyl 4-chlorobenzoate, R2=piperidine, R3=H]

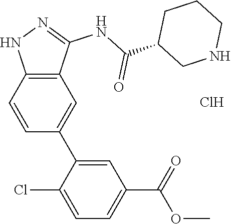 [SMILES: COC(=O)c1ccc(Cl)c(-c2ccc3[nH]nc(NC(=O)[C@@H]4CCCNC4)c3c2)c1 ; Cl]

Obtained 0.020 g (69% of yield). ¹H NMR (500 MHz, DMSO-d₆) δ ppm 1.65-1.91 (m, 3H), 2.08 (m, 1H), 2.84-3.02 (m, 2H), 3.05-3.13 (m, 1H), 3.14-3.20 (m, 1H), 3.87 (s, 3H), 7.42 (dd, J=8.62, 1.45 Hz, 1H), 7.55 (d, J=8.69 Hz, 1H), 7.75 (d, J=8.39 Hz, 1H), 7.86 (s, 1H), 7.92 (d, J=2.13 Hz, 1H), 7.96 (dd, J=8.31, 2.21 Hz, 1H), 8.56-8.76 (m, 2H), 10.74 (s, 1H), 12.91 (br. s., 1H). HRMS (ESI) calcd for C21H22ClN4O3 [M+H]⁺ 413.1375, found 413.1378.

### (3R)-N-[5-(2,4-Difluorophenyl)-1H-indazol-3-yl]piperidine-3-carboxamide hydrochloride (cpd 30) [(I), R1=2,4-difluorophenyl, R2=piperidine, R3=H]

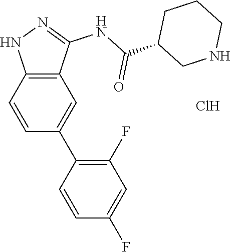 [SMILES: O=C(Nc1n[nH]c2ccc(-c3ccc(F)cc3F)cc12)[C@@H]1CCCNC1 ; Cl]

Obtained 0.029 g (99% of yield). ¹H NMR (500 MHz, DMSO-d₆) δ ppm 1.64-1.90 (m, 3H), 2.10 (m, 1H), 2.85-3.04 (m, 2H), 3.05-3.14 (m, 1H), 3.18 (d, J=11.90 Hz, 1H), 7.21 (td, J=8.43, 2.21 Hz, 1H), 7.37 (m, 1H), 7.47 (m, 1H), 7.52-7.58 (m, 2H), 7.90 (s, 1H), 8.69 (br. s., 2H), 10.72 (s, 1H), 12.87 (br. s., 1H). HRMS (ESI) calcd for C19H19F2N4O [M+H]⁺ 357.1522, found 357.1524.

### (3R)-N-{5-[4-Methoxy-2-(trifluoromethyl)phenyl]-1H-indazol-3-yl}piperidine-3-carboxamide hydrochloride (cpd 31) [(I), R1=4-Methoxy-2-(trifluoromethyl)phenyl, R2=piperidine, R3=H]

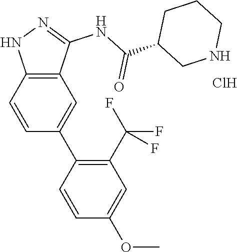 [SMILES: COc1ccc(-c2ccc3[nH]nc(NC(=O)[C@@H]4CCCNC4)c3c2)c(C(F)(F)F)c1 ; Cl]

Obtained 0.026 g (82% of yield). ¹H NMR (500 MHz, DMSO-d₆) δ ppm 1.67-1.84 (m, 3H), 2.07 (m, 1H), 2.90 (m, 1H), 2.93-3.00 (m, 2H), 3.07 (q, J=10.01 Hz, 1H), 3.16 (d, J=11.90 Hz, 1H), 3.32 (d, J=11.44 Hz, 1H), 3.88 (s, 3H), 7.25 (d, J=8.69 Hz, 1H), 7.27-7.32 (m, 2H), 7.33 (m, 1H), 7.48 (d, J=8.69 Hz, 1H), 7.68 (s, 1H), 8.51-8.77 (m, 2H), 10.68 (s, 1H), 12.84 (br. s., 1H). HRMS (ESI) calcd for C21H22F3N4O2 [M+H]⁺ 419.1690, found 419.1703.

### (3R)-N-[5-(2-Fluoro-5-methylphenyl)-1H-indazol-3-yl]piperidine-3-carboxamide hydrochloride (cpd 32) [(I), R1=2-fluoro-5-methylphenyl, R2=piperidine, R3=H]

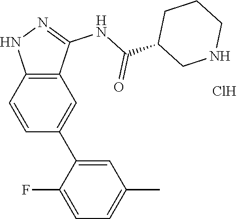 [SMILES: Cc1ccc(F)c(-c2ccc3[nH]nc(NC(=O)[C@@H]4CCCNC4)c3c2)c1 ; Cl]

Obtained 0.020 g (96% of yield). ¹H NMR (500 MHz, DMSO-d₆) δ ppm 1.64-1.92 (m, 3H), 2.11 (m, 1H), 2.35 (s, 3H), 2.83-3.02 (m, 2H), 3.06-3.13 (m, 1H), 3.18 (d, J=12.20 Hz, 1H), 7.18-7.20 (m, 2H), 7.29 (d, J=7.78 Hz, 1H), 7.45-7.56 (m, 2H), 7.90 (s, 1H), 8.59-8.79 (m, 2H), 10.70 (s, 1H), 12.85 (br. s., 1H). HRMS (ESI) calcd for C20H22FN4O [M+H]⁺ 353.1772, found 353.1777.

### (3R)-N-[5-(5-Acetyl-2-chlorophenyl)-1H-indazol-3-yl]piperidine-3-carboxamide hydrochloride (cpd 33) [(I), R1=5-acetyl-2-chlorophenyl, R2=piperidine, R3=H]

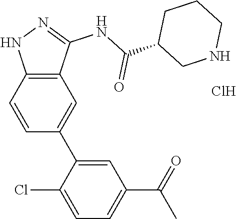 [SMILES: CC(=O)c1ccc(Cl)c(-c2ccc3[nH]nc(NC(=O)[C@@H]4CCCNC4)c3c2)c1 ; Cl]

Obtained 0.027 g (94% of yield). ¹H NMR (500 MHz, DMSO-d₆) δ ppm 1.62-1.89 (m, 3H), 2.05-2.14 (m, 1H), 2.62 (s, 3H), 2.88-3.00 (m, 2H), 3.09 (q, J=9.91 Hz, 1H), 3.16 (d, J=11.29 Hz, 1H), 3.31-3.36 (m, 1H), 7.43 (dd, J=8.69, 1.53 Hz, 1H), 7.56 (d, J=8.69 Hz, 1H), 7.75 (d, J=8.24 Hz, 1H), 7.85 (s, 1H), 7.92 (d, J=2.14 Hz, 1H), 7.97 (dd, J=8.39, 2.29 Hz, 1H), 8.64 (br. s., 1H), 8.71 (br. s., 1H), 10.74 (s, 1H), 12.91 (br. s., 1H). HRMS (ESI) calcd for C21H22ClN4O2 [M+H]⁺ 397.1426, found 397.1426.

### (3R)-N-[5-(5-Amino-2-chlorophenyl)-1H-indazol-3-yl]piperidine-3-carboxamide dihydrochloride (cpd 34) [(I), R1=5-amino-2-chlorophenyl, R2=piperidine, R3=H]

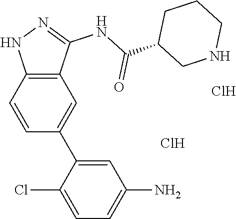 [SMILES: Nc1ccc(Cl)c(-c2ccc3[nH]nc(NC(=O)[C@@H]4CCCNC4)c3c2)c1 ; Cl ; Cl]

Obtained 0.021 g (81% of yield). 1H NMR (500 MHz, DMSO-d₆) δ ppm 1.67-1.86 (m, 3H), 2.05-2.16 (m, 1H), 2.87-2.95 (m, 1H), 2.99 (m, 1H), 3.05-3.12 (m, 1H), 3.17 (d, J=12.05 Hz, 1H), 6.88-6.96 (m, 2H), 7.32-7.42 (m, 2H), 7.51 (m, 1H), 7.77 (s, 1H), 8.66-8.75 (m, 1H), 8.77-8.88 (m, 1H), 10.73 (s, 1H), 12.87 (br. s., 1H). HRMS (ESI) calcd for C19H21ClN5O [M+H]⁺ 370.1429, found 370.1439.

### (3R)-N-[5-(2,5-Difluorophenyl)-1H-indazol-3-yl]piperidine-3-carboxamide hydrochloride (cpd 35) [(I), R1=2,5-difluorophenyl, R2=piperidine, R3=H]

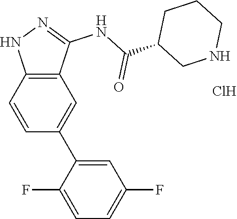 [SMILES: O=C(Nc1n[nH]c2ccc(-c3cc(F)ccc3F)cc12)[C@@H]1CCCNC1 ; Cl]

Obtained 0.024 g (99% of yield). ¹H NMR (500 MHz, DMSO-d₆) δ ppm 1.64-1.90 (m, 3H), 2.09 (m, 1H), 2.82-3.03 (m, 2H), 3.07-3.15 (m, 1H), 3.18 (d, J=11.90 Hz, 1H), 7.20-7.29 (m, 1H), 7.33-7.42 (m, 2H), 7.51-7.58 (m, 2H), 7.97 (s, 1H), 8.67 (br. s., 2H), 10.73 (s, 1H), 12.90 (br. s., 1H). HRMS (ESI) calcd for C19H19F2N4O [M+H]⁺ 357.1522, found 357.1525.

### (3R)-N-{5-[4-Methyl-2-(trifluoromethyl)phenyl]-1H-indazol-3-yl}piperidine-3-carboxamide hydrochloride (cpd 36) [(I), R1=4-methyl-2-(trifluoromethyl)phenyl, R2=piperidine, R3=H]

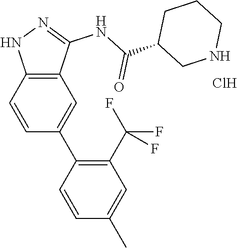 [SMILES: Cc1ccc(-c2ccc3[nH]nc(NC(=O)[C@@H]4CCCNC4)c3c2)c(C(F)(F)F)c1 ; Cl]

Obtained 0.009 g (90% of yield). ¹H NMR (500 MHz, DMSO-d₆) δ ppm 1.64-1.89 (m, 3H), 2.07 (m, 1H), 2.44 (s, 3H), 2.83-2.93 (m, 1H), 2.94-3.00 (m, 1H), 3.04-3.10 (m, 1H), 3.15 (d, J=12.81 Hz, 1H), 3.30-3.34 (m, 1H), 7.26 (d, J=8.69 Hz, 1H), 7.30 (d, J=7.63 Hz, 1H), 7.43 (d, J=8.54 Hz, 1H), 7.52 (d, J=7.78 Hz, 1H), 7.64 (s, 1H), 7.69 (s, 1H), 8.52-8.75 (m, 2H), 10.68 (s, 1H), 12.85 (br. s., 1H). HRMS (ESI) calcd for C21H22F3N4O [M+H]⁺ 403.1740, found 403.1743.

### Methyl 4-chloro-3-(3-{[(3R)-pyrrolidin-3-ylcarbonyl]amino}-1H-indazol-5-yl)benzoate hydrochloride (cpd 37) [(I), R1=methyl 4-chlorobenzoate, R2=pyrrolidine, R3=H]

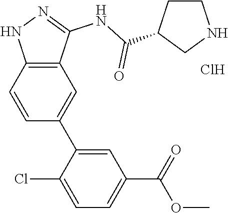 [SMILES: COC(=O)c1ccc(Cl)c(-c2ccc3[nH]nc(NC(=O)[C@@H]4CCNC4)c3c2)c1 ; Cl]

Obtained 0.026 g (88% of yield). ¹H NMR (500 MHz, DMSO-d₆) δ ppm 2.05-2.35 (m, 2H), 3.15-3.30 (m, 2H), 3.87 (s, 3H), 7.42 (dd, J=8.62, 1.60 Hz, 1H), 7.55 (d, J=8.69 Hz, 1H), 7.75 (d, J=8.39 Hz, 1H), 7.90-7.93 (m, 2H), 7.96 (dd, J=8.39, 2.14 Hz, 1H), 8.85-9.08 (m, 2H), 10.83 (s, 1H), 12.93 (br. s., 1H). HRMS (ESI) calcd for C20H2OClN4O3 [M+H]⁺ 399.1219, found 399.1219.

### (3R)-N-[5-(5-Acetyl-2-chlorophenyl)-1H-indazol-3-yl]pyrrolidine-3-carboxamide hydrochloride (cpd 38) [(I), R1=5-acetyl-2-chlorophenyl, R2=pyrrolidine, R3=H]

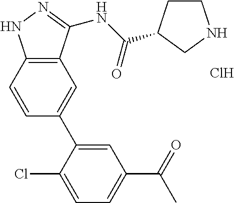 [SMILES: CC(=O)c1ccc(Cl)c(-c2ccc3[nH]nc(NC(=O)[C@@H]4CCNC4)c3c2)c1 ; Cl]

Obtained 0.021 g (89% of yield). 1H NMR (500 MHz, DMSO-d₆) δ ppm 2.05-2.35 (m, 2H), 2.62 (s, 3H), 3.15-3.30 (m, 2H), 7.43 (dd, J=8.69, 1.68 Hz, 1H), 7.56 (d, J=8.69 Hz, 1H), 7.75 (d, J=8.39 Hz, 1H), 7.89-7.93 (m, 2H), 7.97 (dd, J=8.31, 2.21 Hz, 1H), 8.80-9.11 (m, 2H), 10.82 (s, 1H), 12.92 (br. s., 1H). HRMS (ESI) calcd for C20H2OClN4O2 [M+H]⁺ 383.1270, found 383.1276.

### (3R)-N-{5-[2-Chloro-5-(methylcarbamoyl)phenyl]-1H-indazol-3-yl}pyrrolidine-3-carboxamide hydrochloride (cpd 39) [(I), R1=2-chloro-5-(methylcarbamoyl)phenyl, R2=pyrrolidine, R3=H]

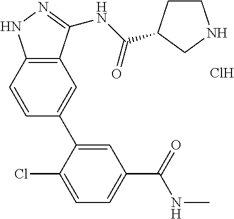 [SMILES: CNC(=O)c1ccc(Cl)c(-c2ccc3[nH]nc(NC(=O)[C@@H]4CCNC4)c3c2)c1 ; Cl]

Obtained 0.018 g (75% of yield). ¹H NMR (500 MHz, DMSO-d₆) δ ppm 2.04-2.18 (m, 1H), 2.26-2.35 (m, 1H), 2.78 (d, J=4.58 Hz, 3H), 3.23 (td, J=14.03, 6.56 Hz, 2H), 7.43 (dd, J=8.69, 1.53 Hz, 1H), 7.55 (d, J=8.54 Hz, 1H), 7.67 (d, J=8.24 Hz, 1H), 7.84 (dd, J=8.31, 2.21 Hz, 1H), 7.88 (d, J=2.14 Hz, 1H), 7.90 (d, J=0.76 Hz, 1H), 8.62 (q, J=4.17 Hz, 1H), 8.82-9.12 (m, 2H), 10.82 (s, 1H), 12.91 (br. s., 1H). HRMS (ESI) calcd for C20H21ClN5O2 [M+H]⁺ 398.1379, found 398.1386.

### Methyl 4-cyano-3-(3-{[(3R)-piperidin-3-ylcarbonyl]amino}-1H-indazol-5-yl)benzoate hydrochloride (cpd 40) [(I), R1=methyl 4-cyanobenzoate, R2=piperidine, R3=H]

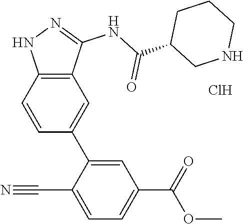 [SMILES: COC(=O)c1ccc(C#N)c(-c2ccc3[nH]nc(NC(=O)[C@@H]4CCCNC4)c3c2)c1 ; Cl]

Obtained 0.009 g (84% of yield). 1H NMR (500 MHz, DMSO-d₆) δ ppm 1.63-1.93 (m, 3H), 2.04-2.16 (m, 1H), 2.90-3.03 (m, 2H), 3.10 (q, J=9.81 Hz, 1H), 3.17 (d, J=11.74 Hz, 1H), 3.92 (s, 3H), 7.57-7.68 (m, 2H), 8.04 (s, 1H), 8.07-8.09 (m, 2H), 8.12 (d, J=8.69 Hz, 1H), 8.52-8.84 (m, 2H), 10.80 (s, 1H), 13.01 (br. s., 1H). HRMS (ESI) calcd for C22H22N5O3 [M+H]⁺ 404.1717, found 404.1717.

### (3R)-N-[5-(2-Chloro-5-hydroxyphenyl)-1H-indazol-3-yl]piperidine-3-carboxamide hydrochloride (cpd 41) [(I), R1=2-chloro-5-hydroxyphenyl, R2=piperidine, R3=H]

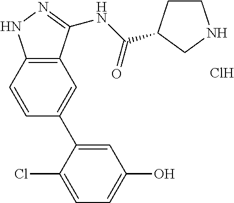 [SMILES: O=C(Nc1n[nH]c2ccc(-c3cc(O)ccc3Cl)cc12)[C@@H]1CCNC1 ; Cl]

Obtained 0.017 g (85% of yield). ¹H NMR (500 MHz, DMSO-d₆) δ ppm 1.65-1.92 (m, 3H), 2.07-2.12 (m, 1H), 2.88-2.94 (m, 1H), 2.95-3.03 (m, 1H), 3.10 (q, J=9.91 Hz, 1H), 3.16 (d, J=12.20 Hz, 1H), 3.31-3.36 (m, 1H), 6.75-6.82 (m, 2H), 7.32 (d, J=7.47 Hz, 1H), 7.37 (dd, J=8.69, 1.53 Hz, 1H), 7.49 (d, J=9.15 Hz, 1H), 7.76 (s, 1H), 8.57-8.85 (m, 2H), 9.83 (br. s., 1H), 10.70 (s, 1H), 12.84 (br. s., 1H). HRMS (ESI) calcd for C19H2OClN4O2 [M+H]⁺ 371.1270, found 371.1279.

### Methyl 2,4-dichloro-5-(3-{[(3R)-piperidin-3-ylcarbonyl]amino}-1H-indazol-5-yl)benzoate hydrochloride (cpd 42) [(I), R1=methyl 2,4-dichlorobenzoate, R2=piperidine, R3=H]

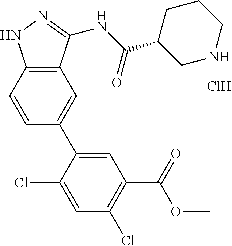 [SMILES: COC(=O)c1cc(-c2ccc3[nH]nc(NC(=O)[C@@H]4CCCNC4)c3c2)c(Cl)cc1Cl ; Cl]

Obtained 0.016 g (89% of yield). ¹H NMR (500 MHz, DMSO-d₆) δ ppm 1.64-1.89 (m, 3H), 2.06-2.14 (m, 1H), 2.86-3.00 (m, 2H), 3.03-3.14 (m, 2H), 3.17 (d, J=12.20 Hz, 1H), 3.87 (s, 3H), 7.41 (dd, J=8.62, 1.30 Hz, 1H), 7.55 (d, J=8.69 Hz, 1H), 7.81-7.86 (m, 2H), 7.93 (s, 1H), 8.51-8.81 (m, 2H), 10.74 (s, 1H), 12.93 (br. s., 1H). HRMS (ESI) calcd for C21H21C12N4O3 [M+H]⁺ 447.0985, found 447.0989.

### (3R)-N-[5-(2-Chloro-5-propanoylphenyl)-1H-indazol-3-yl]piperidine-3-carboxamide hydrochloride (cpd 43) [(I), R1=2-chloro-5-propanoylphenyl, R2=piperidine, R3=H]

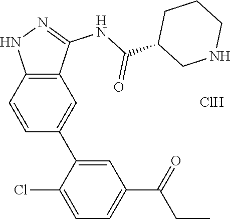 [SMILES: CCC(=O)c1ccc(Cl)c(-c2ccc3[nH]nc(NC(=O)[C@@H]4CCCNC4)c3c2)c1 ; Cl]

Obtained 0.007 g (93% of yield). ¹H NMR (500 MHz, DMSO-d₆) δ ppm 1.09 (m, 3H), 1.64-1.89 (m, 3H), 2.08 (d, J=12.35 Hz, 1H), 2.91-3.02 (m, 2H), 3.09 (m, 2H), 3.16 (d, J=11.29 Hz, 1H), 3.31-3.36 (m, 2H), 7.43 (dd, J=8.62, 1.45 Hz, 1H), 7.55 (d, J=8.54 Hz, 1H), 7.74 (d, J=8.24 Hz, 1H), 7.85 (s, 1H), 7.92 (d, J=1.98 Hz, 1H), 7.98 (dd, J=8.31, 2.21 Hz, 1H), 8.58-8.80 (m, 2H), 10.74 (s, 1H), 12.91 (br. s., 1H). HRMS (ESI) calcd for C22H24ClN4O2 [M+H]⁺ 411.1583, found 411.1591.

### (3R)-N-{5-[2-Chloro-4-(trifluoromethyl)phenyl]-1H-indazol-3-yl}piperidine-3-carboxamide hydrochloride (cpd 44) [(I), R1=2-chloro-4-(trifluoromethyl)phenyl, R2=piperidine, R3=H]

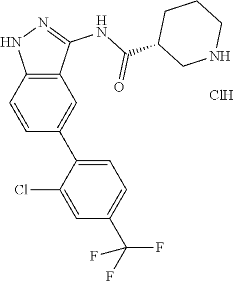 [SMILES: O=C(Nc1n[nH]c2ccc(-c3ccc(C(F)(F)F)cc3Cl)cc12)[C@@H]1CCCNC1 ; Cl]

Obtained 0.027 g (98% of yield). ¹H NMR (500 MHz, DMSO-d₆) δ ppm 1.65-1.91 (m, 3H), 2.02-2.16 (m, 1H), 2.88-2.93 (m, 1H), 2.99 (m, 1H), 3.09 (q, J=9.81 Hz, 1H), 3.17 (d, J=12.35 Hz, 1H), 3.30-3.34 (m, 2H), 7.45 (dd, J=8.62, 1.60 Hz, 1H), 7.57 (d, J=8.69 Hz, 1H), 7.66 (d, J=7.93 Hz, 1H), 7.82 (dd, J=8.01, 1.14 Hz, 1H), 7.87 (s, 1H), 8.00 (d, J=1.07 Hz, 1H), 8.42-8.79 (m, 2H), 10.76 (s, 1H), 12.93 (br. s., 1H). HRMS (ESI) calcd for C20H19ClF3N4O [M+H]⁺ 423.1194, found 423.1205.

### (3R)-N-{5-[4-Chloro-2-(trifluoromethyl)phenyl]-1H-indazol-3-yl}piperidine-3-carboxamide hydrochloride (cpd 45) [(I), R1=4-chloro-2-(trifluoromethyl)phenyl, R2=piperidine, R3=H]

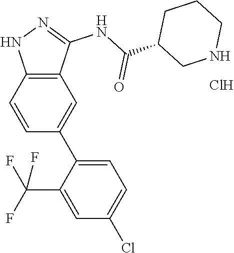 [SMILES: O=C(Nc1n[nH]c2ccc(-c3ccc(Cl)cc3C(F)(F)F)cc12)[C@@H]1CCCNC1 ; Cl]

Obtained 0.030 g (99% of yield). ¹H NMR (500 MHz, DMSO-d₆) δ ppm 1.63-1.89 (m, 3H), 2.06-2.10 (m, 1H), 2.86-2.92 (m, 1H), 2.97 (m, 1H), 3.07 (q, J=10.17 Hz, 1H), 3.16 (d, J=11.59 Hz, 1H), 3.31-3.34 (m, 1H), 7.28 (d, J=8.85 Hz, 1H), 7.46 (d, J=8.24 Hz, 1H), 7.51 (d, J=8.69 Hz, 1H), 7.73 (s, 1H), 7.82 (dd, J=8.24, 2.14 Hz, 1H), 7.91 (d, J=2.14 Hz, 1H), 8.55-8.77 (m, 2H), 10.72 (s, 1H), 12.90 (br. s., 1H). HRMS (ESI) calcd for C20H19ClF3N4O [M+H]⁺ 423.1194, found 423.1201.

### (3R)-N-{5-[5-Fluoro-2-(trifluoromethyl)phenyl]-1H-indazol-3-yl}piperidine-3-carboxamide hydrochloride (cpd 46) [(I), R1=5-fluoro-2-(trifluoromethyl)phenyl, R2=piperidine, R3=H]

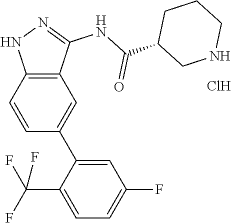 [SMILES: O=C(Nc1n[nH]c2ccc(-c3cc(F)ccc3C(F)(F)F)cc12)[C@@H]1CCCNC1 ; Cl]

Obtained 0.028 g (98% of yield). ¹H NMR (500 MHz, DMSO-d₆) δ ppm 1.62-1.91 (m, 3H), 2.02-2.14 (m, 1H), 2.87-2.92 (m, 1H), 2.96 (m, 1H), 3.07 (q, J=9.76 Hz, 1H), 3.16 (d, J=12.20 Hz, 1H), 3.31-3.36 (m, 1H), 7.29-7.32 (m, 2H), 7.45-7.50 (m, 1H), 7.52 (d, J=8.54 Hz, 1H), 7.76 (s, 1H), 7.92 (dd, J=8.92, 5.41 Hz, 1H), 8.49-8.78 (m, 1H), 10.72 (s, 1H), 12.90 (br. s., 1H). HRMS (ESI) calcd for C20H19F4N4O [M+H]⁺ 407.1490, found 407.1499.

### (3R)-N-[5-(2-Chlorophenyl)-1H-indazol-3-yl]piperidine-3-carboxamide hydrochloride (cpd 47) [(I), R1=2-chlorophenyl, R2=piperidine, R3=H]

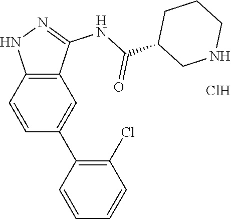 [SMILES: O=C(Nc1n[nH]c2ccc(-c3ccccc3Cl)cc12)[C@@H]1CCCNC1 ; Cl]

Obtained 0.018 g (86% of yield). ¹H NMR (500 MHz, DMSO-d₆) δ ppm 1.69-1.86 (m, 3H), 2.10 (d, J=8.24 Hz, 1H), 2.87-3.02 (m, 2H), 3.03-3.21 (m, 2H), 3.32-3.34 (m, 1H), 7.37-7.46 (m, 4H), 7.52 (d, J=8.69 Hz, 1H), 7.57 (d, J=7.63 Hz, 1H), 7.80 (s, 1H), 8.51-8.80 (m, 2H), 10.71 (s, 1H), 12.86 (br. s., 1H). HRMS (ESI) calcd for C19H2OClN4O [M+H]⁺ 355.1320, found 355.1326.

### (3R)-N-{5-[2-Chloro-5-(propan-2-yl)phenyl]-1H-indazol-3-yl}piperidine-3-carboxamide hydrochloride (cpd 48) [(I), R1=2-chloro-5-(propan-2-yl)phenyl, R2=piperidine, R3=H]

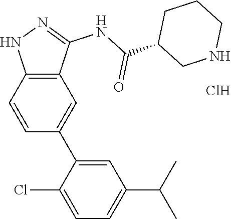 [SMILES: CC(C)c1ccc(Cl)c(-c2ccc3[nH]nc(NC(=O)[C@@H]4CCCNC4)c3c2)c1 ; Cl]

Obtained 0.020 g (91% of yield). 1H NMR (500 MHz, DMSO-d₆) δ ppm 1.22 (d, J=7.02 Hz, 6H), 1.56-1.95 (m, 3H), 2.07-2.12 (m, 1H), 2.84-3.02 (m, 3H), 3.05-3.13 (m, 1H), 3.16 (d, J=12.96 Hz, 1H), 3.32-3.36 (m, 1H), 7.25-7.31 (m, 2H), 7.37 (dd, J=8.69, 1.37 Hz, 1H), 7.47 (d, J=8.24 Hz, 1H), 7.52 (d, J=8.69 Hz, 1H), 7.78 (s, 1H), 8.54-8.85 (m, 2H), 10.70 (s, 1H), 12.85 (br. s., 1H). HRMS (ESI) calcd for C22H26ClN4O [M+H]⁺ 397.1790, found 397.1798.

### (3R)-N-(5-Phenyl-1H-indazol-3-yl)piperidine-3-carboxamide hydrochloride (cpd 49) [(I), R1=phenyl, R2=piperidine, R3=H]

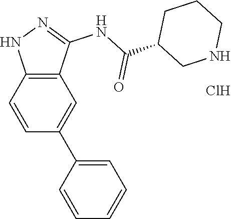 [SMILES: O=C(Nc1n[nH]c2ccc(-c3ccccc3)cc12)[C@@H]1CCCNC1 ; Cl]

Obtained 0.018 g (90% of yield). ¹H NMR (500 MHz, DMSO-d₆) δ ppm 1.69-1.91 (m, 3H), 2.10-2.18 (m, 1H), 2.90-3.02 (m, 2H), 3.08-3.15 (m, 1H), 3.19 (d, J=11.74 Hz, 1H), 7.35 (t, J=7.47 Hz, 1H), 7.47 (t, J=7.93 Hz, 2H), 7.54 (d, J=8.54 Hz, 1H), 7.61-7.67 (m, 3H), 7.99 (s, 1H), 8.71 (d, J=19.98 Hz, 2H), 10.70 (s, 1H), 12.81 (br. s., 1H). HRMS (ESI) calcd for C19H21N4O [M+H]⁺ 321.1710, found 321.1719.

### Methyl 2,4-difluoro-5-(3-{[(3R)-piperidin-3-ylcarbonyl]amino}-1H-indazol-5-yl)benzoate hydrochloride (cpd 50) [(I), R1=methyl 2,4-difluorobenzoate, R2=piperidine, R3=H]

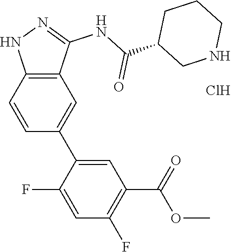 [SMILES: COC(=O)c1cc(-c2ccc3[nH]nc(NC(=O)[C@@H]4CCCNC4)c3c2)c(F)cc1F ; Cl]

Obtained 0.011 g (93% of yield). ¹H NMR (500 MHz, DMSO-d₆) δ ppm 1.62-1.90 (m, 3H), 2.03-2.18 (m, 1H), 2.89-3.04 (m, 2H), 3.10 (q, J=9.61 Hz, 1H), 3.18 (d, J=11.59 Hz, 1H), 3.33-3.38 (m, 2H), 3.88 (s, 3H), 7.49 (d, J=9.50 Hz, 1H), 7.55-7.66 (m, 2H), 7.93-8.04 (m, 2H), 8.72 (d, J=16.47 Hz, 2H), 10.75 (s, 1H), 12.92 (br. s., 1H). HRMS (ESI) calcd for C21H21F2N4O3 [M+H]⁺ 415.1576, found 415.1584.

### (3R)-N-[5-(5-tert-Butyl-2-chlorophenyl)-1H-indazol-3-yl]piperidine-3-carboxamide hydrochloride (cpd 51) [(I), R1=5-tert-Butyl-2-chlorophenyl, R2=piperidine, R3=H]

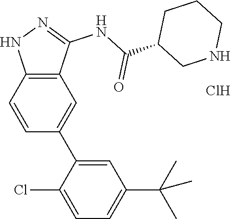 [SMILES: CC(C)(C)c1ccc(Cl)c(-c2ccc3[nH]nc(NC(=O)[C@@H]4CCCNC4)c3c2)c1 ; Cl]

Obtained 0.016 g (70% of yield). ¹H NMR (500 MHz, DMSO-d₆) δ ppm 1.31 (s, 9H), 1.65-1.92 (m, 3H), 2.06-2.15 (m, 1H), 2.89-3.01 (m, 2H), 3.05-3.11 (m, 1H), 3.16 (d, J=12.35 Hz, 1H), 3.32-3.35 (m, 1H), 7.37-7.44 (m, 3H), 7.47 (d, J=8.39 Hz, 1H), 7.52 (d, J=8.69 Hz, 1H), 7.79 (s, 1H), 8.54-8.78 (m, 2H), 10.70 (s, 1H), 12.85 (br. s., 1H). HRMS (ESI) calcd for C23H28ClN4O [M+H]⁺ 411.1946, found 411.1956.

### (3R)-N-[5-(2-Chloro-5-fluorophenyl)-1H-indazol-3-yl]piperidine-3-carboxamide hydrochloride (cpd 52) [(I), R1=2-chloro-5-fluorophenyl, R2=piperidine, R3=H]

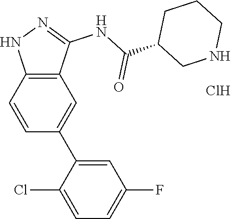 [SMILES: O=C(Nc1n[nH]c2ccc(-c3cc(F)ccc3Cl)cc12)[C@@H]1CCCNC1 ; Cl]

Obtained 0.021 g (99% of yield). ¹H NMR (500 MHz, DMSO-d₆) δ ppm 1.69-1.92 (m, 3H), 2.10-2.15 (m, 1H), 2.92-2.97 (m, 1H), 3.01 (m, 1H), 3.09-3.14 (m, 1H), 3.20 (d, J=12.20 Hz, 1H), 3.36-3.41 (m, 1H), 7.29-7.37 (m, 2H), 7.45 (dd, J=8.69, 1.53 Hz, 1H), 7.57 (d, J=8.69 Hz, 1H), 7.66 (dd, J=8.54, 5.19 Hz, 1H), 7.87 (s, 1H), 8.58-8.85 (m, 2H), 10.76 (s, 1H), 12.93 (br. s., 1H). HRMS (ESI) calcd for C19H19ClFN4O [M+H]⁺ 373.1226, found 373.1222.

### (3R)-N-[5-(2,5-Dicyanophenyl)-1H-indazol-3-yl]piperidine-3-carboxamide hydrochloride (cpd 53) [(I), R1=2,5-dicyanophenyl, R2=piperidine, R3=H]

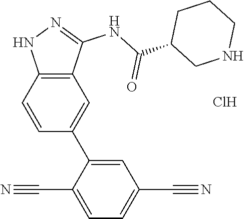 [SMILES: N#Cc1ccc(C#N)c(-c2ccc3[nH]nc(NC(=O)[C@@H]4CCCNC4)c3c2)c1 ; Cl]

Obtained 0.008 g (80% of yield). ¹H NMR (500 MHz, DMSO-d₆) δ ppm 1.62-1.91 (m, 3H), 2.04-2.15 (m, 1H), 2.86-3.04 (m, 2H), 3.06-3.15 (m, 1H), 3.18 (d, J=12.66 Hz, 1H), 7.57-7.66 (m, 2H), 8.04 (s, 1H), 8.05 (dd, J=8.08, 1.53 Hz, 1H), 8.15 (d, J=1.22 Hz, 1H), 8.19 (d, J=8.08 Hz, 1H), 8.54-8.77 (m, 2H), 10.80 (s, 1H), 13.03 (br. s., 1H). HRMS (ESI) calcd for C21H₁₉N₆O [M+H]⁺ 371.1615, found 371.1618.

### Methyl 2-amino-4-chloro-5-(3-{[(3R)-pyrrolidin-3-ylcarbonyl]amino}-1H-indazol-5-yl)benzoate dihydrochloride (cpd 54) [(I), R1=methyl 2-amino-4-chlorobenzoate, R2=pyrrolidine, R3=H]

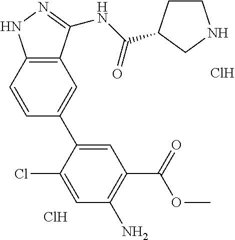 [SMILES: COC(=O)c1cc(-c2ccc3[nH]nc(NC(=O)[C@@H]4CCNC4)c3c2)c(Cl)cc1N ; Cl ; Cl]

Obtained 0.028 g (74% of yield). ¹H NMR (500 MHz, DMSO-d₆) δ ppm 2.06-2.35 (m, 2H), 3.14-3.33 (m, 3H), 3.79 (s, 3H), 7.01 (s, 1H), 7.31 (dd, J=8.62, 1.45 Hz, 1H), 7.46 (d, J=8.69 Hz, 1H), 7.67 (s, 1H), 7.75 (d, J=0.76 Hz, 1H), 8.88-9.14 (m, 2H), 10.76 (s, 1H), 12.82 (br. s., 1H). HRMS (ESI) calcd for C20H21ClN5O3 [M+H]⁺ 414.1328, found 414.1339.

### (3R)-N-{5-[2-Cyano-5-(methylcarbamoyl)phenyl]-1H-indazol-3-yl}piperidine-3-carboxamide hydrochloride (cpd 55) [(I), R1=2-cyano-5-(methylcarbamoyl)phenyl, R2=piperidine, R3=H]

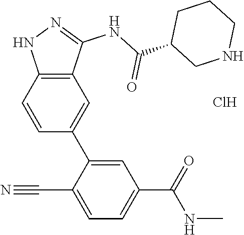 [SMILES: CNC(=O)c1ccc(C#N)c(-c2ccc3[nH]nc(NC(=O)[C@@H]4CCCNC4)c3c2)c1 ; Cl]

Obtained 0.016 g (88% of yield). ¹H NMR (500 MHz, DMSO-d₆) δ ppm 1.60-1.91 (m, 3H), 2.07-2.17 (m, 1H), 2.82 (d, J=4.58 Hz, 3H), 2.89-2.95 (m, 1H), 3.00 (m, 1H), 3.08-3.13 (m, 1H), 3.14-3.22 (m, 1H), 7.58-7.66 (m, 2H), 7.96 (dd, J=8.08, 1.68 Hz, 1H), 8.01-8.04 (m, 2H), 8.06 (d, J=8.08 Hz, 1H), 8.63 (br. s., 2H), 8.81 (m, 1H), 10.79 (s, 1H), 12.99 (br. s., 1H). HRMS (ESI) calcd for C22H23N6O2 [M+H]⁺ 403.1877, found 403.1879.

### Propan-2-yl 4-chloro-3-(3-{[(3R)-piperidin-3-ylcarbonyl]amino}-1H-indazol-5-yl)benzoate hydrochloride (cpd 56) [(I), R1=propan-2-yl 4-chlorobenzoate, R2=piperidine, R3=H]

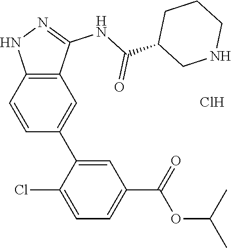 [SMILES: CC(C)OC(=O)c1ccc(Cl)c(-c2ccc3[nH]nc(NC(=O)[C@@H]4CCCNC4)c3c2)c1 ; Cl]

Obtained 0.018 g (67% of yield). ¹H NMR (500 MHz, DMSO-d₆) δ ppm 1.32 (d, J=6.25 Hz, 6H), 1.66-1.89 (m, 3H), 2.06-2.14 (m, 1H), 2.87-3.00 (m, 2H), 3.04-3.13 (m, 1H), 3.16 (d, J=11.90 Hz, 1H), 3.34-3.36 (m, 1H), 5.15 (quin, J=6.25 Hz, 1H), 7.41 (dd, J=8.69, 1.53 Hz, 1H), 7.56 (d, J=8.69 Hz, 1H), 7.74 (d, J=8.24 Hz, 1H), 7.84 (s, 1H), 7.89 (d, J=2.14 Hz, 1H), 7.94 (dd, J=8.31, 2.21 Hz, 1H), 8.59-8.78 (m, 2H), 10.74 (s, 1H), 12.91 (br. s., 1H). HRMS (ESI) calcd for C23H26ClN4O3 [M+H]⁺ 441.1688, found 441.1693.

### Methyl 2,4-dichloro-3-(3-{[(3R)-piperidin-3-ylcarbonyl]amino}-1H-indazol-5-yl)benzoate hydrochloride (cpd 57) [(I), R1=methyl 2,4-dichlorobenzoate, R2=piperidine, R3=H]

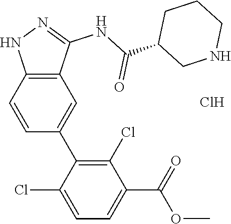 [SMILES: COC(=O)c1ccc(Cl)c(-c2ccc3[nH]nc(NC(=O)[C@@H]4CCCNC4)c3c2)c1Cl ; Cl]

Obtained 0.026 g (90% of yield). ¹H NMR (500 MHz, DMSO-d₆) δ ppm 1.64-1.85 (m, 3H), 2.04-2.12 (m, 1H), 2.86-2.99 (m, 2H), 3.04-3.12 (m, 1H), 3.15 (d, J=12.81 Hz, 1H), 3.29-3.35 (m, 1H), 3.88 (s, 3H), 7.20 (d, J=9.30 Hz, 1H), 7.56 (d, J=8.69 Hz, 1H), 7.70-7.73 (m, 2H), 7.78 (d, J=8.85 Hz, 1H), 8.55-8.78 (m, 1H), 10.73 (s, 1H), 12.92 (br. s., 1H). HRMS (ESI) calcd for C21H21C12N4O3 [M+H]⁺ 447.0985, found 447.0990.

### (3R)-N-{5-[2-Chloro-5-(1,2,4-oxadiazol-3-yl)phenyl]-1H-indazol-3-yl}piperidine-3-carboxamide dihydrochloride (cpd 58) [(I), R1=3-(4-chlorophenyl)-1,2,4-oxadiazole, R2=piperidine, R3=H]

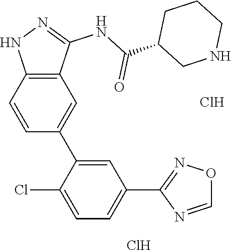 [SMILES: O=C(Nc1n[nH]c2ccc(-c3cc(-c4ncon4)ccc3Cl)cc12)[C@@H]1CCCNC1 ; Cl ; Cl]

Obtained 0.014 g (94% of yield). ¹H NMR (500 MHz, DMSO-d₆) δ ppm 1.65-1.91 (m, 3H), 2.01-2.16 (m, 1H), 2.84-3.01 (m, 2H), 3.02-3.18 (m, 2H), 3.33-3.36 (m, 1H), 7.47 (dd, J=8.69, 1.53 Hz, 1H), 7.57 (d, J=8.69 Hz, 1H), 7.82 (d, J=8.39 Hz, 1H), 7.90 (s, 1H), 8.01 (d, J=1.98 Hz, 1H), 8.05 (dd, J=8.39, 2.14 Hz, 1H), 8.57-8.78 (m, 2H), 9.78 (s, 1H), 10.75 (s, 1H), 12.93 (br. s., 1H). HRMS (ESI) calcd for C21H2OClN602 [M+H]⁺ 423.1331, found 423.1340.

### (3R)-N-{5-[2-Chloro-5-(1,3,4-oxadiazol-2-yl)phenyl]-1H-indazol-3-yl}piperidine-3-carboxamide trifluoroacetate (cpd 59) [(I), R1=2-(4-chlorophenyl)-1,3,4-oxadiazole, R2=piperidine, R3=H]

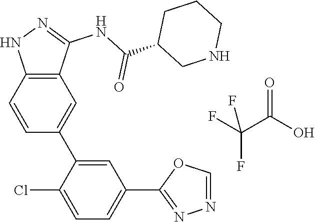

Obtained 0.004 g (13% of yield). ¹H NMR (500 MHz, DMSO-d₆) δ ppm 1.67-1.86 (m, 3H), 2.08-2.11 (m, 1H), 2.90-2.98 (m, 2H), 3.08-3.20 (m, 2H), 7.45 (m, 1H), 7.57 (m, 1H), 7.72 (m, 1H), 7.84-7.93 (m, 3H), 8.13 (s, 1H), 8.51 (br. s., 2H), 10.74 (s, 1H), 12.91 (br. s., 1H).). HRMS (ESI) calcd for C21H2OClN6O2 [M+H]⁺ 423.1331, found 423.1331.

### (3R)-N-{5-[2-Chloro-5-(5-methyl-1,2,4-oxadiazol-3-yl)phenyl]-1H-indazol-3-yl}piperidine-3-carboxamide dihydrochloride (cpd 60) [(I), R1=3-(4-chlorophenyl)-5-methyl-1,2,4-oxadiazole, R2=piperidine, R3=H]

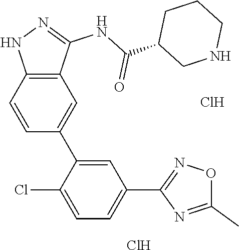 [SMILES: Cc1nc(-c2ccc(Cl)c(-c3ccc4[nH]nc(NC(=O)[C@@H]5CCCNC5)c4c3)c2)no1 ; Cl ; Cl]

Obtained 0.020 g (60% of yield). ¹H NMR (500 MHz, DMSO-d₆) δ ppm 1.66-1.84 (m, 3H) 2.02-2.15 (m, 1H) 2.68 (s, 3H), 2.84-2.93 (m, 1H), 2.98 (m, 1H), 3.05-3.13 (m, 1H), 3.14-3.22 (m, 1H), 7.45 (dd, J=8.08, 1.53 Hz, 1H), 7.57 (d, J=8.69 Hz, 1H), 7.79 (d, J=8.39 Hz, 1H), 7.89 (s, 1H), 7.96 (d, J=1.98 Hz, 1H), 8.00 (dd, J=8.39, 2.14 Hz, 1H), 8.63 (d, J=16.01 Hz, 2H), 10.75 (s, 1H), 12.92 (br. s., 1H).). HRMS (ESI) calcd for C22H22ClN6O2 [M+H]⁺437.1488, found 437.1488.

### N-[5-(2-Chloro-5-cyanophenyl)-1H-indazol-3-yl]piperidine-4-carboxamide hydrochloride (cpd 61) [(I), R1=2-chloro-5-cyanophenyl, R2=piperidine, R3=H]

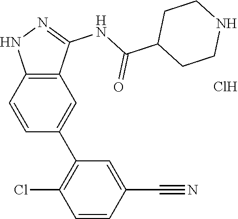 [SMILES: N#Cc1ccc(Cl)c(-c2ccc3[nH]nc(NC(=O)C4CCNCC4)c3c2)c1 ; Cl]

Obtained 0.029 g (85% of yield). ¹H NMR (500 MHz, DMSO-d₆) δ ppm 1.73-1.92 (m, 2H), 2.02 (d, J=12.05 Hz, 2H), 2.77 (tt, J=10.98, 3.51 Hz, 1H), 2.85-3.01 (m, 2H), 3.32-3.36 (m, 2H), 7.43 (dd, J=8.77, 1.30 Hz, 1H), 7.55 (d, J=8.69 Hz, 1H), 7.82 (d, J=8.40 Hz, 1H), 7.84 (s, 1H), 7.89 (dd, J=8.40, 2.00 Hz, 1H), 7.93 (d, J=1.98 Hz, 1H), 8.41 (q, J=8.80 Hz, 1H), 8.73 (d, J=10.07 Hz, 1H), 10.58 (s, 1H), 12.90 (br. s., 1H). HRMS (ESI) calcd for C20H19ClN5O [M+H]⁺ 380.1273, found 380.1281.

### N-[5-(2-Chloro-4-hydroxyphenyl)-1H-indazol-3-yl]-3-(dimethylamino)cyclobutanecarboxamide hydrochloride (cpd 62) [(I), R1=2-chloro-4-hydroxyphenyl, R2=(dimethylamino)cyclobutane, R3=H]

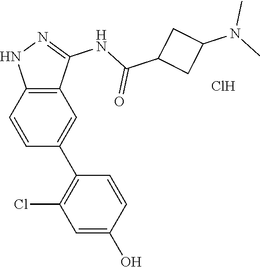 [SMILES: CN(C)C1CC(C(=O)Nc2n[nH]c3ccc(-c4ccc(O)cc4Cl)cc23)C1 ; Cl]

Obtained 0.028 g (90% of yield). ¹H NMR (500 MHz, DMSO-d₆) δ ppm 2.37-2.44 (m, 2H), 2.67 (d, J=4.88 Hz, 6H), 3.04 (m, 1H), 3.65 (m, 1H), 6.83 (dd, J=8.39, 2.44 Hz, 1H), 6.94 (d, J=2.44 Hz, 1H), 7.20 (d, J=8.39 Hz, 1H), 7.34 (dd, J=8.62, 1.45 Hz, 1H), 7.47 (d, J=8.69 Hz, 1H), 7.73 (s, 1H), 9.71-10.16 (m, 2H), 10.51 (s, 1H), 12.77 (br. s., 1H). HRMS (ESI) calcd for C20H22ClN4O2 [M+H]⁺ 385.1426, found 385.1428.

### N-[5-(2-Chloro-4-hydroxyphenyl)-1H-indazol-3-yl]-3-(methylamino)cyclobutanecarboxamide hydrochloride (cpd 63) [(I), R1=2-chloro-4-hydroxyphenyl, R2=(methylamino)cyclobutane, R3=H]

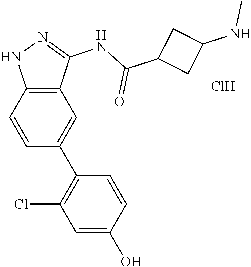 [SMILES: CNC1CC(C(=O)Nc2n[nH]c3ccc(-c4ccc(O)cc4Cl)cc23)C1 ; Cl]

Obtained 0.039 g (75% of yield). ¹H NMR (500 MHz, DMSO-d₆) δ ppm 2.26-2.36 (m, 2H), 2.41-2.48 (m, 4H), 3.12 (m, 1H), 3.60-3.69 (m, 1H), 3.81 (m, 1H), 6.83 (dd, J=8.31, 2.52 Hz, 1H), 6.94 (d, J=2.44 Hz, 1H), 7.22 (d, J=8.40 Hz, 1H), 7.33 (dd, J=8.62, 1.30 Hz, 1H), 7.47 (d, J=8.54 Hz, 1H), 7.73 (s, 1H), 8.67-8.93 (m, 2H), 9.99 (br. s., 1H), 10.49 (m, 1H), 12.76 (br. s., 1H). HRMS (ESI) calcd for C19H2OClN4O2 [M+H]⁺ 371.1270, found 371.1273.

### N-[5-(2-Chloro-6-fluorophenyl)-1H-indazol-3-yl]-1-methylpiperidine-4-carboxamide hydrochloride (cpd 64) [(XV), R1=2-chloro-6-fluorophenyl, R2=1-methylpiperidine, R3=H]

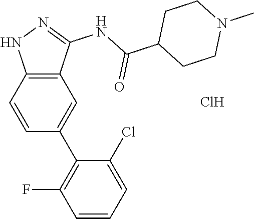 [SMILES: CN1CCC(C(=O)Nc2n[nH]c3ccc(-c4c(F)cccc4Cl)cc23)CC1 ; Cl]

Obtained 0.027 g (80% of yield). 1H NMR (500 MHz, DMSO-d₆) δ ppm 1.79-1.92 (m, 2H), 2.04-2.10 (m, 2H), 2.70 (t, J=11.90 Hz, 1H), 2.76 (d, J=4.73 Hz, 3H), 2.92-3.02 (m, 2H), 3.47 (m, J=11.59 Hz, 2H), 7.29 (d, J=9.00 Hz, 1H), 7.32-7.39 (m, 1H), 7.42-7.50 (m, 2H), 7.55 (d, J=8.54 Hz, 1H), 7.77 (s, 1H), 9.40-10.08 (m, 1H), 10.45-10.76 (m, 1H), 12.89 (br. s., 1H). HRMS (ESI) calcd for C20H21ClFN4O [M+H]⁺ 387.1383, found 387.1386.

### 1-Butyl-N-[5-(2-chloro-6-fluorophenyl)-1H-indazol-3-yl]piperidine-4-carboxamide hydrochloride (cpd 65) [(I), R1=2-chloro-6-fluorophenyl, R2=1-butylpiperidine, R3=H]

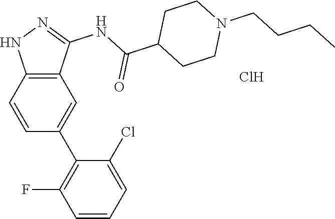 [SMILES: CCCCN1CCC(C(=O)Nc2n[nH]c3ccc(-c4c(F)cccc4Cl)cc23)CC1 ; Cl]

Obtained 0.031 g (89% of yield). ¹H NMR (500 MHz, DMSO-d₆) δ ppm 0.88-0.95 (m, 3H), 1.26-1.36 (m, 2H), 1.58-1.71 (m, 2H), 1.90 (q, J=13.42 Hz, 2H), 2.03-2.13 (m, 2H), 2.73 (t, J=12.12 Hz, 1H), 2.93 (q, J=10.88 Hz, 2H), 2.99-3.07 (m, 2H), 3.51-3.57 (m, 2H), 7.29 (d, J=7.93 Hz, 1H), 7.33-7.37 (m, 1H), 7.45-7.50 (m, 2H), 7.55 (d, J=8.54 Hz, 1H), 7.77 (s, 1H), 9.43 (br. s., 1H), 10.60 (s, 1H), 12.89 (br. s., 1H). HRMS (ESI) calcd for C23H27ClFN4O [M+H]⁺ 429.1852, found 429.1855.

### 1-(3-Aminopropyl)-N-[5-(2-chloro-6-fluorophenyl)-1H-indazol-3-yl]piperidine-4-carboxamide dihydrochloride (cpd 66) [(I), R1=2-chloro-6-fluorophenyl, R2 1-(3-aminopropyl)piperidine, R3=H]

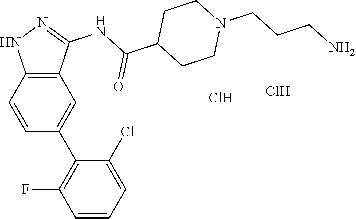 [SMILES: NCCCN1CCC(C(=O)Nc2n[nH]c3ccc(-c4c(F)cccc4Cl)cc23)CC1 ; Cl ; Cl]

Obtained 0.034 g (93% of yield). ¹H NMR (500 MHz, DMSO-d₆) δ ppm 1.89-2.05 (m, 3H), 2.09 (d, J=12.66 Hz, 3H), 2.75 (m, J=12.28, 1H), 2.82-3.03 (m, 4H), 3.10-3.23 (m, 2H), 3.53 (d, J=11.29 Hz, 2H), 7.29 (d, J=9.00 Hz, 1H), 7.32-7.40 (m, 1H), 7.43-7.48 (m, 2H), 7.55 (d, J=8.69 Hz, 1H), 7.76 (s, 1H), 8.00 (br. s., 3H), 10.11 (br. s., 1H), 10.61 (s, 1H), 12.89 (br. s., 1H). HRMS (ESI) calcd for C22H26ClFN5O [M+H]⁺ 430.1805, found 430.1802.

### N-[5-(2-Chloro-6-fluorophenyl)-1H-indazol-3-yl]-1-(1-methoxypropan-2-yl)piperidine-4-carboxamide hydrochloride (cpd 67) [(I), R1=2-chloro-6-fluorophenyl, R2=1-(1-methoxypropan-2-yl)piperidine, R3=H]

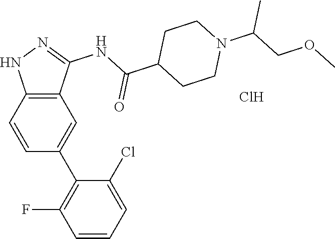 [SMILES: COCC(C)N1CCC(C(=O)Nc2n[nH]c3ccc(-c4c(F)cccc4Cl)cc23)CC1 ; Cl]

Obtained 0.018 g (80% of yield). ¹H NMR (500 MHz, DMSO-d₆) δ ppm 1.27 (d, J=6.56 Hz, 3H), 1.92-2.03 (m, 2H), 2.05-2.14 (m, 2H), 2.74 (t, J=12.05 Hz, 1H), 3.00-3.12 (m, 2H), 3.32 (s, 3H), 3.45-3.51 (m, 2H), 3.54-3.65 (m, 3H), 7.27-7.31 (m, 1H), 7.32-7.38 (m, 1H), 7.43-7.50 (m, 2H), 7.55 (d, J=8.69 Hz, 1H), 7.73-7.78 (m, 1H), 9.14-9.59 (m, 1H), 10.51-10.68 (m, 1H), 12.89 (br. s., 1H). HRMS (ESI) calcd for C23H27ClFN4O2 [M+H]⁺ 445.1801, found 445.1799.

### N-[5-(2,6-Dichlorophenyl)-1H-indazol-3-yl]-1-methylpiperidine-4-carboxamide (cpd 68) [(I), R1=2,6-dichlorophenyl, R2=1-methylpiperidine, R3=H]

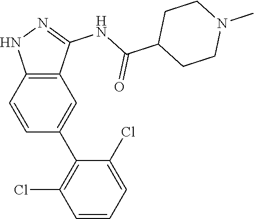 [SMILES: CN1CCC(C(=O)Nc2n[nH]c3ccc(-c4c(Cl)cccc4Cl)cc23)CC1]

After completion of the reaction, the suspension was diluted with DCM and washed with aqueous NaHCO₃ and brine. The organic phase was dried with anhydrous Na₂SO₄, filtered and evaporated to dryness. The crude was purified by flash column chromatography (DCM/MeOH/33% aqueous ammonia 90:10:1) to obtain 0.026 g (41% of yield). 1H NMR (500 MHz, DMSO-d₆) δ ppm 1.56-1.70 (m, 2H), 1.77 (d, J=12.35 Hz, 2H), 1.87 (br. s., 2H), 2.15 (s, 3H), 2.40 (br. s., 1H), 2.80 (d, J=10.37 Hz, 2H), 7.17 (d, J=8.69 Hz, 1H), 7.41-7.46 (m, 1H), 7.48-7.54 (m, 1H), 7.59 (d, J=8.08 Hz, 2H), 7.69 (s, 1H), 10.36 (s, 1H), 12.78 (s, 1H). HRMS (ESI) calcd for C20H21C12N4O [M+H]⁺ 403.1087, found 403.1084.

### 1-Butyl-N-[5-(2,6-dichlorophenyl)-1H-indazol-3-yl]piperidine-4-carboxamide (cpd 69) [(I), R1=2,6-dichlorophenyl, R2=1-butylpiperidine, R3=H]

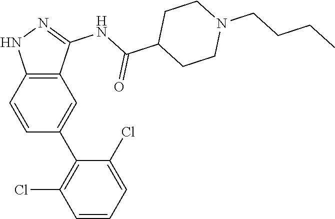 [SMILES: CCCCN1CCC(C(=O)Nc2n[nH]c3ccc(-c4c(Cl)cccc4Cl)cc23)CC1]

After completion of the reaction, the suspension was diluted with DCM and washed with aqueous NaHCO₃ and brine. The organic phase was dried with anhydrous Na₂SO₄, filtered and evaporated to dryness. The crude was purified by flash column chromatography (DCM/MeOH/33% aqueous ammonia 90:10:0.5) to obtain 0.048 g (53% of yield). 1H NMR (401 MHz, DMSO-d₆) δ ppm 0.87 (t, J=7.26 Hz, 3H), 1.22-1.33 (m, 2H), 1.36-1.44 (m, 2H), 1.57-1.70 (m, 2H), 1.78 (d, J=11.60 Hz, 2H), 1.83-1.92 (m, 2H), 2.25 (t, J=6.96 Hz, 2H), 2.37-2.47 (m, 1H), 2.88 (d, J=11.23 Hz, 2H), 7.17 (dd, J=8.54, 1.59 Hz, 1H), 7.43 (dd, J=8.67, 7.57 Hz, 1H), 7.52 (dd, J=8.67, 0.61 Hz, 1H), 7.57-7.61 (m, 2H), 7.69 (s, 1H), 10.31 (s, 1H), 12.75 (s, 1H). HRMS (ESI) calcd for C23H27C12N4O [M+H]⁺ 445.1557, found 445.1560.

### N-[5-(2,6-Dichlorophenyl)-1H-indazol-3-yl]-1-(2-methylpropyl)piperidine-4-carboxamide (cpd 70) [(I), R1=2,6-dichlorophenyl, R2=1-(2-methylpropyl)piperidine, R3=H]

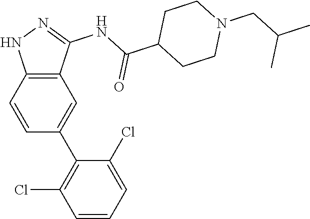 [SMILES: CC(C)CN1CCC(C(=O)Nc2n[nH]c3ccc(-c4c(Cl)cccc4Cl)cc23)CC1]

After completion of the reaction, the suspension was diluted with DCM and washed with aqueous NaHCO₃ and brine. The organic phase was dried with anhydrous Na₂SO₄, filtered and evaporated to dryness. The crude was purified by flash column chromatography (DCM/MeOH/33% aqueous ammonia 90:10:0.5) to obtain 0.041 g (63% of yield). ¹H NMR (401 MHz, DMSO-d₆) δ ppm 0.84 (d, J=6.59 Hz, 6H), 1.56-1.81 (m, 5H), 1.86 (t, J=11.11 Hz, 2H), 2.01 (d, J=7.08 Hz, 2H), 2.38-2.48 (m, 1H), 2.85 (d, J=10.86 Hz, 2H), 7.17 (d, J=8.67 Hz, 1H), 7.40-7.46 (m, 1H), 7.52 (d, J=8.67 Hz, 1H), 7.59 (d, J=8.06 Hz, 2H), 7.69 (s, 1H), 10.31 (s, 1H), 12.75 (s, 1H). HRMS (ESI) calcd for C23H27Cl2N4O [M+H]⁺ 445.1557, found 445.1559.

### N-[5-(2,6-Dichlorophenyl)-1H-indazol-3-yl]-1-(3-hydroxy-2,2-dimethylpropyl)piperidine-4-carboxamide (cpd 71) [(I), R1=2,6-dichlorophenyl, R2=1-(3-hydroxy-2,2-dimethylpropyl)piperidine, R3=H]

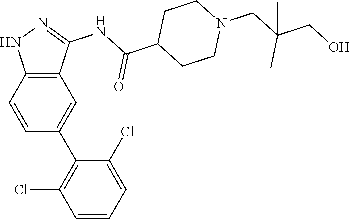 [SMILES: CC(C)(CO)CN1CCC(C(=O)Nc2n[nH]c3ccc(-c4c(Cl)cccc4Cl)cc23)CC1]

After completion of the reaction, the suspension was diluted with DCM and washed with aqueous NaHCO₃ and brine. The organic phase was dried with anhydrous Na₂SO₄, filtered and evaporated to dryness. The crude was purified by flash column chromatography (DCM/MeOH/33% aqueous ammonia 90:10:0.5) to obtain 0.017 g (35% of yield). ¹H NMR (401 MHz, DMSO-d₆) δ ppm 0.77 (s, 6H), 1.58-1.82 (m, 4H), 2.01-2.29 (m, 4H), 2.40 (br. s., 1H), 2.84 (d, J=9.64 Hz, 2H), 3.15 (s, 2H), 4.55 (br. s., 1H), 7.18 (dd, J=8.61, 1.53 Hz, 1H), 7.43 (dd, J=8.67, 7.57 Hz, 1H), 7.52 (d, J=8.79 Hz, 1H), 7.56-7.62 (m, 2H), 7.68 (br. s, 1H), 10.31 (s, 1H), 12.75 (s, 1H). HRMS (ESI) calcd for C24H29Cl2N4O2 [M+H]⁺ 475.1662, found 475.1639.

### N-[5-(6-Chloro-2-fluoro-3-methylphenyl)-1H-indazol-3-yl]-1-methylpiperidine-4-carboxamide hydrochloride (cpd 72) [(I), R1=6-chloro-2-fluoro-3-methylphenyl, R2=1-methylpiperidine, R3=H]

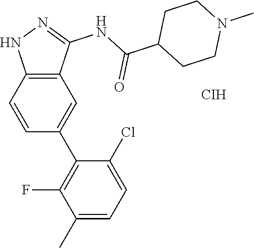

Obtained 0.030 g (79% of yield). ¹H NMR (500 MHz, DMSO-d₆) δ ppm 1.81-1.91 (m, 2H), 2.03-2.10 (m, 2H), 2.27 (d, J=1.98 Hz, 3H), 2.65-2.73 (m, 1H), 2.76 (d, J=4.58 Hz, 3H), 2.92-3.02 (m, 2H), 3.47 (d, J=12.66 Hz, 2H), 7.26 (d, J=9.15 Hz, 1H), 7.31-7.39 (m, 2H), 7.54 (d, J=8.69 Hz, 1H), 7.75 (s, 1H), 9.65 (br. s., 1H), 10.60 (s, 1H), 12.86 (br. s., 1H). HRMS (ESI) calcd for C21H23ClFN4O [M+H]⁺ 401.1539, found 401.1546.

### N-[5-(2-Chloro-6-fluoro-3-methylphenyl)-1H-indazol-3-yl]-1-methylpiperidine-4-carboxamide hydrochloride (cpd 73) [(I), R1=2-chloro-6-fluoro-3-methylphenyl, R2=1-methylpiperidine, R3=H]

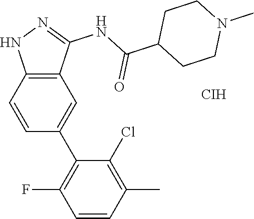

Obtained 0.037 g (90% of yield). ¹H NMR (500 MHz, DMSO-d₆) δ ppm 1.78-1.92 (m, 2H), 2.04-2.11 (m, 2H), 2.36-2.39 (m, 3H), 2.70 (t, J=11.90 Hz, 1H), 2.76 (d, J=4.73 Hz, 3H), 2.92-3.04 (m, 2H), 3.46 (d, J=11.90 Hz, 2H), 7.20-7.29 (m, 2H), 7.44 (dd, J=8.46, 6.02 Hz, 1H), 7.54 (d, J=8.69 Hz, 1H), 7.73 (s, 1H), 9.65 (br. s., 1H), 10.53-10.65 (m, 1H), 12.87 (br. s., 1H). HRMS (ESI) calcd for C21H23ClFN4O [M+H]⁺ 401.1539, found 401.1539.

### N-[5-(5-Chloro-2-fluoro-3-methylphenyl)-1H-indazol-3-yl]-1-methylpiperidine-4-carboxamide hydrochloride (cpd 74) [(I), R1=5-chloro-2-fluoro-3-methylphenyl, R2=1-methylpiperidine, R3=H]

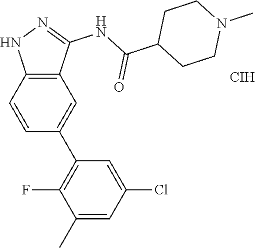

Obtained 0.032 g (71% of yield). ¹H NMR (500 MHz, DMSO-d₆) δ ppm 1.83-1.97 (m, 2H), 2.09 (d, J=14.03 Hz, 2H), 2.30 (d, J=1.37 Hz, 3H), 2.72 (tt, J=12.12, 3.30 Hz, 1H), 2.77 (d, J=4.58 Hz, 3H), 2.93-3.05 (m, 2H), 3.49 (d, J=10.37 Hz, 2H), 7.35 (dd, J=6.25, 2.44 Hz, 1H), 7.40 (dd, J=5.80, 2.44 Hz, 1H), 7.47-7.52 (m, 2H), 7.52-7.57 (m, 1H), 7.93 (s, 1H), 9.66 (br. s., 1H), 10.61 (s, 1H), 12.86 (br. s., 1H). HRMS (ESI) calcd for C21H23ClFN4O [M+H]⁺ 401.1539, found 401.1541.

### N-{5-[2-Methoxy-5-(propan-2-yl)phenyl]-1H-indazol-3-yl}-1-methylpiperidine-4-carboxamide hydrochloride (cpd 75) [(I), R1=2-methoxy-5-(propan-2-yl)phenyl, R2=1-methylpiperidine, R3=H]

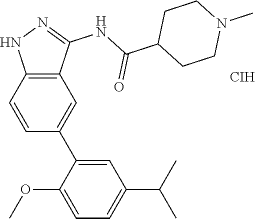

Obtained 0.035 g (89% of yield). ¹H NMR (500 MHz, DMSO-d₆) δ ppm 1.21 (d, J=6.86 Hz, 6H), 1.89 (qd, J=13.30, 3.80 Hz, 2H), 2.08 (d, J=14.18 Hz, 2H), 2.71 (tt, J=12.10, 3.40 Hz, 1H), 2.77 (d, J=4.58 Hz, 3H), 2.89 (m, J=6.90 Hz, 1H), 2.93-3.04 (m, 2H), 3.47 (t, J=11.20 Hz, 2H), 3.71 (s, 3H), 7.03 (d, J=8.39 Hz, 1H), 7.11 (d, J=2.14 Hz, 1H), 7.20 (dd, J=8.46, 2.06 Hz, 1H), 7.39-7.47 (m, 2H), 7.72 (s, 1H), 9.65 (br. s., 1H), 10.50 (s, 1H), 12.71 (br. s., 1H). HRMS (ESI) calcd for C24H31N4O2 [M+H]⁺ 407.2442, found 407.2443.

### 1-Methyl-N-{5-[3-(trifluoromethyl)phenyl]-1H-indazol-3-yl}piperidine-4-carboxamide hydrochloride (cpd 76) [(I), R1=3-(trifluoromethyl)phenyl, R2=1-methylpiperidine, R3=H]

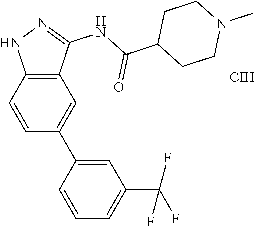

Obtained 0.017 g (95% of yield). ¹H NMR (500 MHz, DMSO-d₆) δ ppm 1.92 (qd, J=13.00, 2.90 Hz, 2H), 2.12 (d, J=13.88 Hz, 2H), 2.74 (tt, J=12.10, 3.60 Hz, 3H), 2.78 (d, J=4.42 Hz, 3H), 2.94-3.06 (m, 2H), 3.48 (t, J=13.40 Hz, 2H), 7.57 (d, J=8.85 Hz, 1H), 7.65-7.77 (m, 3H), 791-7.98 (m, 3H), 9.71 (br. s., 1H), 10.62 (s, 1H), 12.85 (br. s., 1H). HRMS (ESI) calcd for C21H22F3N4O [M+H]⁺ 403.1740, found 403.1742.

### N-{5-[2-Chloro-5-(hydroxymethyl)phenyl]-1H-indazol-3-yl}-1-methylpiperidine-4-carboxamide p-toluensulfonate (cpd 77) [(I), R1=2-chloro-5-(hydroxymethyl)phenyl, R2=1-methylpiperidine, R3=H]

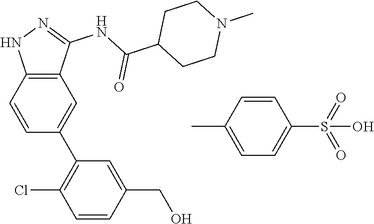 [SMILES: CN1CCC(C(=O)Nc2n[nH]c3ccc(-c4cc(CO)ccc4Cl)cc23)CC1 ; Cc1ccc(S(=O)(=O)O)cc1]

Obtained 0.055 g (96% of yield). ¹H NMR (500 MHz, DMSO-d₆) δ ppm 1.76-1.91 (m, 2H), 2.05-2.13 (m, 2H), 2.28 (s, 8H), 2.71 (t, J=12.12 Hz, 1H), 2.79 (d, J=4.73 Hz, 3H), 2.93-3.05 (m, 2H), 4.54 (s, 2H), 7.26-7.41 (m, 4H), 7.50-7.53 (m, 2H), 7.79 (d, J=0.76 Hz, 1H), 9.14 (br. s., 1H), 10.59 (s, 1H), 12.82 (br. s., 1H). HRMS (ESI) calcd for C21H24ClN4O2 [M+H]⁺ 399.1583, found 399.1585.

### N-[5-(2-Fluorophenyl)-1H-indazol-3-yl]-1-methylpiperidine-4-carboxamide hydrochloride (cpd 78) [(I), R1=2-fluorophenyl, R2=1-methylpiperidine, R3=H]

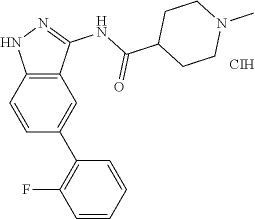

Obtained 0.038 g (86% of yield). ¹H NMR (500 MHz, DMSO-d₆) δ ppm 1.82-1.96 (m, 2H), 2.06-2.13 (m, 2H), 2.66-2.74 (m, 1H), 2.77 (d, J=4.73 Hz, 3H), 2.93-3.05 (m, 2H), 3.48 (d, J=12.20 Hz, 2H), 7.27-7.35 (m, 2H), 7.37-7.44 (m, 1H), 7.47-7.58 (m, 3H), 7.89-8.00 (m, 1H), 9.68 (br. s, 1H), 10.60 (s, 1H), 12.83 (br. s., 1H). HRMS (ESI) calcd for C20H22FN4O [M+H]⁺ 353.1772, found 353.1778.

### N-[5-(2-Cyanophenyl)-1H-indazol-3-yl]-1-methylpiperidine-4-carboxamide p-toluensulfonate (cpd 79) [(I), R1=2-cyanophenyl, R2=1-methylpiperidine, R3=H]

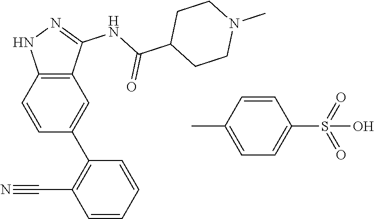

Obtained 0.044 g (90% of yield). ¹H NMR (500 MHz, DMSO-d₆) δ ppm 1.79-1.89 (m, 2H), 2.07-2.13 (m, 2H), 2.28 (s, 3H), 2.68-2.76 (m, 2H), 2.78-2.81 (m, 3H), 2.94-3.05 (m, 2H), 3.50 (d, J=11.90 Hz, 2H), 7.11 (d, J=7.93 Hz, 5H), 7.47 (d, J=7.93 Hz, 5H), 7.52-7.66 (m, 4H), 7.80 (td, J=7.74, 1.30 Hz, 1H), 7.93-7.99 (m, 2H), 9.15 (br. s., 1H), 12.92 (br. s., 1H).). HRMS (ESI) calcd for C21H₂₂N₅O [M+H]⁺ 360.1819, found 360.1821.

### N-[5-(2,5-Dichlorophenyl)-1H-indazol-3-yl]-1-methylpiperidine-4-carboxamide hydrochloride (cpd 80) [(I), R1=2,5-dichlorophenyl, R2=1-methylpiperidine, R3=H]

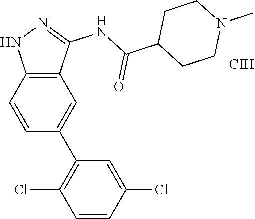

Obtained 0.034 g (79% of yield). ¹H NMR (500 MHz, DMSO-d₆) δ ppm 1.82-1.94 (m, 2H), 2.05-2.12 (m, 2H), 2.71 (t, J=12.05 Hz, 1H), 2.77 (d, J=4.73 Hz, 3H), 2.93-3.03 (m, 2H), 3.48 (d, J=10.83 Hz, 2H), 7.41 (dd, J=8.54, 1.37 Hz, 1H), 7.47-7.50 (m, 2H), 7.53 (d, J=8.69 Hz, 1H), 7.60-7.64 (m, 1H), 7.81-7.89 (m, 1H), 9.62 (br. s., 1H), 10.58-10.64 (m, 1H), 12.88 (br. s., 1H). HRMS (ESI) calcd for C20H21Cl2N4O [M+H]⁺ 403.1087, found 403.1091.

### N-[5-(3-Carbamoylphenyl)-1H-indazol-3-yl]-1-methylpiperidine-4-carboxamide hydrochloride (cpd 81) [(I), R1=3-carbamoylphenyl, R2=1-methylpiperidine, R3=H]

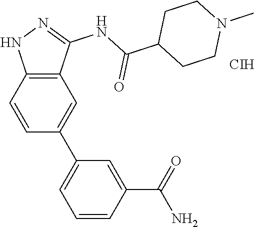

Obtained 0.041 g (82% of yield). ¹H NMR (500 MHz, DMSO-d₆) δ ppm 1.84-1.99 (m, 2H), 2.12 (d, J=13.57 Hz, 2H), 2.71-2.75 (m, 1H), 2.76-2.79 (m, 3H), 2.95-3.05 (m, 2H), 3.50 (d, J=11.74 Hz, 2H), 7.44 (s, 1H), 7.56 (d, J=7.93 Hz, 1H), 7.70-7.74 (m, 1H), 7.77 (d, J=7.78 Hz, 1H), 7.84 (d, J=7.78 Hz, 1H), 8.01-8.07 (m, 1H), 8.09-8.17 (m, 3H), 9.66 (br. s., 1H), 10.62 (s, 1H), 12.80 (br. s., 1H). HRMS (ESI) calcd for C21H24N5O2 [M+H]⁺ 378.1925, found 378.1930.

### N-{5-[4-(Hydroxymethyl)phenyl]-1H-indazol-3-yl}-1-methylpiperidine-4-carboxamide p-toluensulfonate (cpd 82) [(I), R1=4-(hydroxymethyl)phenyl, R2=1-methylpiperidine, R3=H]

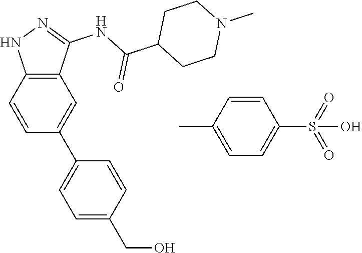

Obtained 0.068 g (98% of yield). ¹H NMR (500 MHz, DMSO-d₆) δ ppm 1.84-1.94 (m, 2H), 2.11 (d, J=13.42 Hz, 2H), 2.28 (s, 3H), 2.70-2.74 (m, 1H), 2.79-2.84 (m, 3H), 2.97-3.06 (m, 2H), 3.50 (d, J=11.44 Hz, 2H), 4.54 (s, 2H), 7.10 (d, J=7.78 Hz, 2H), 7.28-7.66 (m, 9H), 9.16 (br. s., 1H), 10.56 (s, 1H), 12.76 (br. s., 1H). HRMS (ESI) calcd for C21H25N4O2 [M+H]⁺ 365.1972, found 365.1977.

### N-{5-[3-(Hydroxymethyl)phenyl]-1H-indazol-3-yl}-1-methylpiperidine-4-carboxamide p-toluensulfonate (cpd 83) [(I), R1=3-(hydroxymethyl)phenyl, R2=1-methylpiperidine, R3=H]

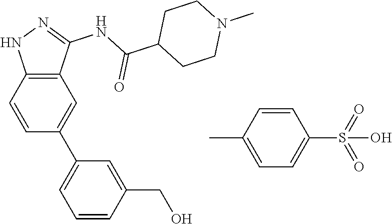 [SMILES: CN1CCC(C(=O)Nc2n[nH]c3ccc(-c4cccc(CO)c4)cc23)CC1 ; Cc1ccc(S(=O)(=O)O)cc1]

Obtained 0.061 g (97% of yield). ¹H NMR (500 MHz, DMSO-d₆) δ ppm 1.79-1.95 (m, 2H), 2.09-2.16 (m, 2H), 2.28 (s, 3H), 2.68-2.76 (m, 1H), 2.79-2.83 (m, 3H), 2.96-3.10 (m, 2H), 3.51 (d, J=11.29 Hz, 2H), 4.58 (s, 2H), 7.98 (s, 1H), 7.10 (d, J=7.78 Hz, 2H), 7.28-7.65 (m, 9H), 9.15 (br. s., 1H), 10.57 (s, 1H), 12.78 (br. s., 1H). HRMS (ESI) calcd for C21H25N4O2 [M+H]⁺ 365.1972, found 365.1970.

### 1-Methyl-N-[5-(pyridin-3-yl)-1H-indazol-3-yl]piperidine-4-carboxamide dihydrochloride (cpd 84) [(I), R1=pyridin-3-yl, R2=1-methylpiperidine, R3=H]

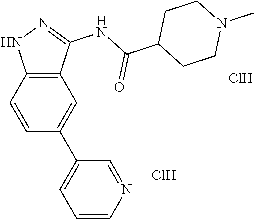 [SMILES: CN1CCC(C(=O)Nc2n[nH]c3ccc(-c4cccnc4)cc23)CC1 ; Cl ; Cl]

Obtained 0.023 g (73% of yield). ¹H NMR (500 MHz, DMSO-d₆) δ ppm 1.89-2.03 (m, 2H), 2.11 (d, J=13.27 Hz, 2H), 2.69-2.80 (m, 1H), 2.76 (d, J=4.73 Hz, 3H), 2.92-3.06 (m, 2H), 7.63 (d, J=8.69 Hz, 1H), 7.79 (dd, J=8.77, 1.45 Hz, 1H), 7.96 (dd, J=7.55, 6.02 Hz, 1H), 8.22 (s, 1H), 8.62 (d, J=7.47 Hz, 1H), 8.78 (d, J=4.88 Hz, 1H), 9.12 (s, 1H), 10.06 (br. s., 1H), 10.71 (s, 1H), 12.98 (br. s., 1H). HRMS (ESI) calcd for C19H22N5O [M+H]⁺ 336.1819, found 336.1817.

### N-[5-(2-Chlorophenyl)-1H-indazol-3-yl]-1-methylpiperidine-4-carboxamide hydrochloride (cpd 85) [(I), R1=2-chlorophenyl, R2=1-methylpiperidine, R3=H]

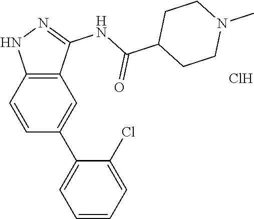 [SMILES: CN1CCC(C(=O)Nc2n[nH]c3ccc(-c4ccccc4Cl)cc23)CC1 ; Cl]

Obtained 0.027 g (78% of yield). ¹H NMR (500 MHz, DMSO-d₆) δ ppm 1.80-1.95 (m, 2H), 2.08 (d, J=13.73 Hz, 2H), 2.71 (tt, J=11.95, 3.60 Hz, 1H), 2.76 (d, J=4.73 Hz, 3H), 2.92-3.05 (m, 2H), 3.47 (d, J=12.51 Hz, 2H), 7.36-7.46 (m, 4H), 7.52 (d, J=8.69 Hz, 1H), 7.57 (ddd, J=7.45, 1.40, 0.70 Hz, 1H), 7.79 (dd, J=1.53, 0.91 Hz, 1H), 9.65 (br. s., 1H), 10.59 (s, 1H), 12.84 (br. s., 1H). HRMS (ESI) calcd for C20H22ClN4O [M+H]⁺ 369.1477, found 369.1483.

### N-[5-(2-Chloro-5-methylphenyl)-1H-indazol-3-yl]-1-methylpiperidine-4-carboxamide hydrochloride (cpd 86) [(I), R1=2-chloro-5-methylphenyl, R2=1-methylpiperidine, R3=H]

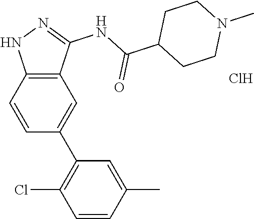 [SMILES: Cc1ccc(Cl)c(-c2ccc3[nH]nc(NC(=O)C4CCN(C)CC4)c3c2)c1 ; Cl]

Obtained 0.029 g (81% of yield). ¹H NMR (500 MHz, DMSO-d₆) δ ppm 1.80-1.94 (m, 2H), 2.04-2.13 (m, 2H), 2.34 (s, 3H), 2.66-2.75 (m, 1H), 2.76 (d, J=4.58 Hz, 3H), 2.93-3.03 (m, 2H), 3.47 (d, J=11.90 Hz, 2H), 7.18-7.26 (m, 2H), 7.37 (dd, J=8.62, 1.30 Hz, 1H), 7.44 (d, J=8.08 Hz, 1H), 7.49-7.54 (m, 1H), 7.77 (d, J=0.61 Hz, 1H), 9.66 (br. s., 1H), 10.58 (s, 1H), 12.82 (br. s., 1H). HRMS (ESI) calcd for C21H24ClN4O [M+H]⁺ 383.1633, found 383.1642.

### N-[5-(4-Fluorophenyl)-1H-indazol-3-yl]-1-methylpiperidine-4-carboxamide hydrochloride (cpd 87) [(I), R1=4-fluorophenyl, R2=1-methylpiperidine, R3=H]

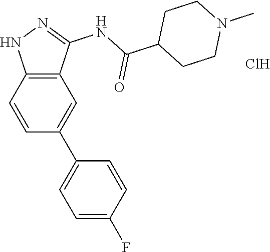 [SMILES: CN1CCC(C(=O)Nc2n[nH]c3ccc(-c4ccc(F)cc4)cc23)CC1 ; Cl]

Obtained 0.038 g (79% of yield). ¹H NMR (500 MHz, DMSO-d₆) δ ppm 1.88-2.03 (m, 2H), 2.14 (d, J=13.42 Hz, 2H), 2.70-2.79 (m, 1H), 2.80-2.83 (m, 3H), 2.98-3.09 (m, 2H), 3.52 (d, J=11.44 Hz, 2H), 7.30-7.38 (m, 2H), 7.56 (d, J=8.69 Hz, 1H), 7.62-7.74 (m, 3H), 7.99 (s, 1H), 9.75 (br. s., 1H), 10.52-10.66 (m, 1H), 12.82 (br. s., 1H). HRMS (ESI) calcd for C20H22FN4O [M+H]⁺ 353.1772, found 353.1773.

### N-[5-(2-Ethylphenyl)-1H-indazol-3-yl]-1-methylpiperidine-4-carboxamide hydrochloride (cpd 88) [(I), R1=2-ethylphenyl, R2=1-methylpiperidine, R3=H]

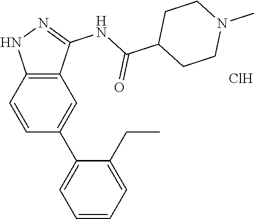 [SMILES: CCc1ccccc1-c1ccc2[nH]nc(NC(=O)C3CCN(C)CC3)c2c1 ; Cl]

Obtained 0.033 g (80% of yield). ¹H NMR (500 MHz, DMSO-d₆) δ ppm 0.99-1.06 (m, 3H), 1.76-1.94 (m, 2H), 2.03-2.11 (m, 2H), 2.53-2.61 (m, 2H), 2.66-2.73 (m, 1H), 2.76 (d, J=4.58 Hz, 3H), 2.92-3.02 (m, 2H), 3.46 (d, J=11.59 Hz, 2H), 7.17 (d, J=7.17 Hz, 1H), 7.22-7.26 (m, 1H), 7.27-7.36 (m, 3H), 7.46-7.52 (m, 1H), 7.62-7.75 (m, 1H), 9.62 (br. s., 1H), 10.55 (s, 1H), 12.77 (br. s., 1H). HRMS (ESI) calcd for C22H27N4O [M+H]⁺ 363.2180, found 363.2181.

### N-[5-(3-Cyanophenyl)-1H-indazol-3-yl]-1-methylpiperidine-4-carboxamide hydrochloride (cpd 89) [(I), R1=3-cyanophenyl, R2=1-methylpiperidine, R3=H]

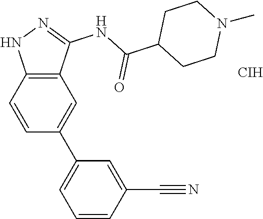

Obtained 0.034 g (81% of yield). ¹H NMR (500 MHz, DMSO-d₆) δ ppm 1.84-2.00 (m, 2H), 2.06-2.16 (m, 2H), 2.69-2.76 (m, 1H), 2.77-2.82 (m, 3H), 2.95-3.06 (m, 2H), 3.50 (d, J=11.74 Hz, 2H), 7.56 (d, J=8.69 Hz, 1H), 7.66-7.70 (m, 1H), 7.71-7.74 (m, 1H), 7.80-7.84 (m, 1H), 7.95-8.02 (m, 1H), 8.06-8.13 (m, 2H), 9.65 (br. s., 1H), 10.52-10.66 (m, 1H), 12.87 (br. s., 1H). HRMS (ESI) calcd for C21H22N5O [M+H]⁺ 360.1819, found 360.1818.

### N-[5-(4-Aminophenyl)-1H-indazol-3-yl]-1-methylpiperidine-4-carboxamide dihydrochloride (cpd 90) [(I), R1=4-aminophenyl, R2=1-methylpiperidine, R3=H]

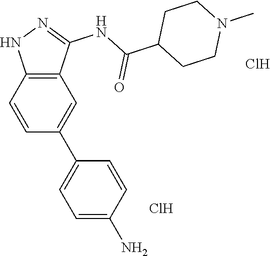 [SMILES: CN1CCC(C(=O)Nc2n[nH]c3ccc(-c4ccc(N)cc4)cc23)CC1 ; Cl ; Cl]

Obtained 0.023 g (76% of yield). ¹H NMR (500 MHz, DMSO-d₆) δ ppm 1.88-2.00 (m, 2H), 2.10 (d, J=13.73 Hz, 2H), 2.70-2.75 (m, 1H), 2.77 (d, J=4.73 Hz, 3H), 2.94-3.05 (m, 2H), 3.49 (d, J=2.14 Hz, 2H), 7.37 (d, J=7.02 Hz, 2H), 7.54 (d, J=8.69 Hz, 1H), 7.64 (d, J=8.54 Hz, 1H), 7.69 (d, J=8.24 Hz, 2H), 7.98-8.04 (m, 1H), 9.82-10.23 (m, 3H), 10.55-10.64 (m, 1H), 12.81 (br. s., 1H). HRMS (ESI) calcd for C20H24N5O [M+H]⁺ 350.1976, found 350.1981.

### N-[5-(2-Aminophenyl)-1H-indazol-3-yl]-1-methylpiperidine-4-carboxamide dihydrochloride (cpd 91) [(I), R1=2-aminophenyl, R2=1-methylpiperidine, R3=H]

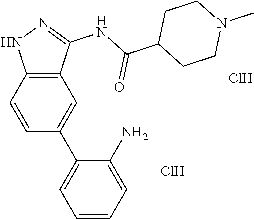 [SMILES: CN1CCC(C(=O)Nc2n[nH]c3ccc(-c4ccccc4N)cc23)CC1 ; Cl ; Cl]

Obtained 0.028 g (79% of yield). ¹H NMR (500 MHz, DMSO-d₆) δ ppm 1.87-1.99 (m, 2H), 2.07-2.15 (m, 2H), 2.70-2.76 (m, 1H), 2.78-2.81 (m, 3H), 2.97-3.06 (m, 2H), 7.12-7.40 (m, 6H), 7.46 (d, J=7.63 Hz, 1H), 7.57 (d, J=8.54 Hz, 1H), 7.77-7.96 (m, 1H), 9.89 (br. s., 1H), 10.57-10.65 (m, 1H), 12.87 (br. s., 1H). HRMS (ESI) calcd for C20H24N5O [M+H]⁺ 350.1976, found 350.1976.

### 1-Methyl-N-[5-(4-sulfamoylphenyl)-1H-indazol-3-yl]piperidine-4-carboxamide hydrochloride (cpd 92) [(I), R1=4-sulfamoylphenyl, R2=1-methylpiperidine, R3=H]

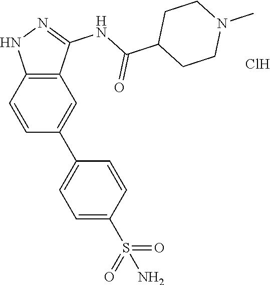 [SMILES: CN1CCC(C(=O)Nc2n[nH]c3ccc(-c4ccc(S(N)(=O)=O)cc4)cc23)CC1 ; Cl]

Obtained 0.036 g (86% of yield). ¹H NMR (500 MHz, DMSO-d₆) δ ppm 1.83-2.00 (m, 2H), 2.11 (d, J=13.88 Hz, 2H), 2.74 (tt, J=12.10, 3.20 Hz, 1H), 2.78 (d, J=4.73 Hz, 3H), 2.89-3.06 (m, 2H), 3.49 (d, J=12.05 Hz, 2H), 7.40 (s, 2H), 7.57 (d, J=8.85 Hz, 1H), 7.71 (dd, J=8.92, 1.30 Hz, 1H), 7.79-7.86 (m, 2H), 7.88-7.93 (m, 2H), 8.09 (s, 1H), 9.77 (br. s., 1H), 10.63 (s, 1H), 12.86 (br. s., 1H). HRMS (ESI) calcd for C20H24N5O3S [M+H]⁺ 414.1595, found 414.1594.

### 1-Methyl-N-(5-phenyl-1H-indazol-3-yl)piperidine-4-carboxamide hydrochloride (cpd 93) [(I), R1=phenyl, R7=R2=1-methylpiperidine, R3=H]

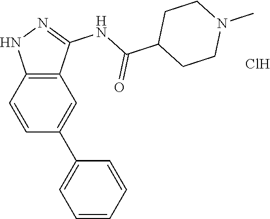 [SMILES: CN1CCC(C(=O)Nc2n[nH]c3ccc(-c4ccccc4)cc23)CC1 ; Cl]

Obtained 0.033 g (93% of yield). 1H NMR (500 MHz, DMSO-d₆) δ ppm 1.86-1.99 (m, 2H), 2.08-2.18 (m, 2H), 2.69-2.76 (m, 1H), 2.77-2.80 (m, 3H), 2.95-3.05 (m, 2H), 3.45-3.54 (m, 2H), 7.31-7.41 (m, 1H), 7.44-7.50 (m, 2H), 7.51-7.56 (m, 1H), 7.59-7.68 (m, 3H), 7.95-8.05 (m, 1H), 9.66-10.03 (m, 1H), 10.53-10.62 (m, 1H), 12.65-13.06 (m, 1H). HRMS (ESI) calcd for C20H23N4O [M+H]⁺ 335.1867, found 335.1865.

### N-{5-[2-Chloro-5-(trifluoromethyl)phenyl]-1H-indazol-3-yl}-1-methylpiperidine-4-carboxamide hydrochloride (cpd 94) [(I), R1=2-chloro-5-(trifluoromethyl)phenyl, R2=1-methylpiperidine, R3=H]

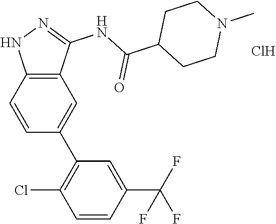 [SMILES: CN1CCC(C(=O)Nc2n[nH]c3ccc(-c4cc(C(F)(F)F)ccc4Cl)cc23)CC1 ; Cl]

Obtained 0.031 g (90% of yield). ¹H NMR (500 MHz, DMSO-d₆) δ ppm 1.82-1.93 (m, 2H), 2.04-2.11 (m, 2H), 2.71 (t, J=11.90 Hz, 1H), 2.76 (d, J=4.42 Hz, 3H), 2.93-3.03 (m, 2H), 3.45-3.52 (m, 2H), 7.41-7.46 (m, 1H), 7.55 (d, J=8.69 Hz, 1H), 7.72-7.76 (m, 1H), 7.77-7.80 (m, 1H), 7.83-7.93 (m, 2H), 9.61 (br. s., 1H), 10.58-10.65 (m, 1H), 12.90 (br. s., 1H). HRMS (ESI) calcd for C21H21ClF3N4O [M+H]⁺ 437.1351, found 437.1359.

### N-[5-(2-Fluoropyridin-3-yl)-1H-indazol-3-yl]-1-methylpiperidine-4-carboxamide dihydrochloride (cpd 95) [(I), R1=2-fluoropyridin-3-yl, R2=1-methylpiperidine, R3=H]

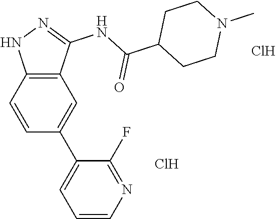 [SMILES: CN1CCC(C(=O)Nc2n[nH]c3ccc(-c4cccnc4F)cc23)CC1 ; Cl ; Cl]

Obtained 0.035 g (92% of yield). ¹H NMR (500 MHz, DMSO-d₆) δ ppm 1.84-1.98 (m, 2H), 2.09 (d, J=13.57 Hz, 2H), 2.68-2.74 (m, 1H), 2.77 (d, J=4.42 Hz, 3H), 2.94-3.06 (m, 2H), 3.47-3.52 (m, 2H), 7.46-7.51 (m, 1H), 7.57 (s, 2H), 8.00 (s, 1H), 8.09 (ddd, J=9.95, 7.66, 1.75 Hz, 1H), 8.23 (d, J=5.19 Hz, 1H), 9.73 (br. s., 1H), 10.64 (s, 1H), 12.90 (br. s., 1H). HRMS (ESI) calcd for C19H21FN5O [M+H]⁺ 354.1725, found 354.1729.

### N-[5-(2-Methoxyphenyl)-1H-indazol-3-yl]-1-methylpiperidine-4-carboxamide hydrochloride (cpd 96) [(I), R1=2-methoxyphenyl, R2=1-methylpiperidine, R3=H]

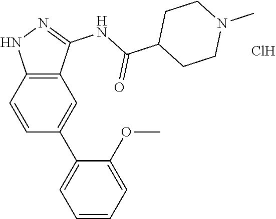 [SMILES: COc1ccccc1-c1ccc2[nH]nc(NC(=O)C3CCN(C)CC3)c2c1 ; Cl]

Obtained 0.034 g (94% of yield). ¹H NMR (500 MHz, DMSO-d₆) δ ppm 1.80-1.96 (m, 2H), 2.08 (d, J=14.03 Hz, 2H), 2.71 (tt, J=12.16, 3.85 Hz, 1H), 2.77 (d, J=4.73 Hz, 3H), 2.87-3.05 (m, 2H), 3.48 (d, J=10.68 Hz, 2H), 3.75 (s, 3H), 7.03 (td, J=7.44, 0.69 Hz, 1H), 7.11 (d, J=8.08 Hz, 1H), 7.26 (dd, J=7.47, 1.37 Hz, 1H), 7.34 (ddd, J=8.20, 7.50, 1.68 Hz, 1H), 7.45 (s, 2H), 7.74 (s, 1H), 9.63 (br. s., 1H), 10.52 (s, 1H), 12.72 (br. s., 1H). HRMS (ESI) calcd for C21H25N4O2 [M+H]⁺ 365.972, found 365.1968.

### N-[5-(2,3-Dichlorophenyl)-1H-indazol-3-yl]-1-methylpiperidine-4-carboxamide hydrochloride (cpd 97) [(I), R1=2,3-dichlorophenyl, R2=1-methylpiperidine, R3=H]

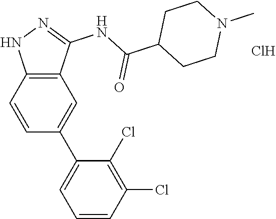 [SMILES: CN1CCC(C(=O)Nc2n[nH]c3ccc(-c4cccc(Cl)c4Cl)cc23)CC1 ; Cl]

Obtained 0.028 g (91% of yield). ¹H NMR (500 MHz, DMSO-d₆) δ ppm 1.77-1.94 (m, 2H), 2.07 (d, J=14.00 Hz, 2H), 2.71 (tt, J=12.32, 3.55 Hz, 1H), 2.76 (d, J=4.58 Hz, 3H), 2.88-3.04 (m, 2H), 3.44-3.52 (m, 2H), 7.36-7.42 (m, 2H), 7.45 (t, J=7.80 Hz, 1H), 7.53 (d, J=8.69 Hz, 1H), 7.67 (dd, J=7.93, 1.68 Hz, 1H), 7.80 (dd, J=1.37, 0.61 Hz, 1H), 9.62 (br. s., 1H), 10.62 (s, 1H), 12.87 (br. s., 1H). HRMS (ESI) calcd for C20H21Cl2N4O [M+H]⁺ 403.1087, found 403.1088.

### N-[5-(2-Chloro-6-methoxyphenyl)-1H-indazol-3-yl]-1-methylpiperidine-4-carboxamide hydrochloride (cpd 98) [(I), R1=2-chloro-6-methoxyphenyl, R2=1-methylpiperidine, R3=H]

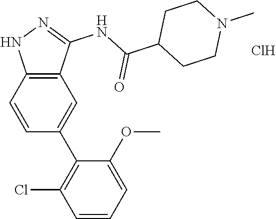 [SMILES: COc1cccc(Cl)c1-c1ccc2[nH]nc(NC(=O)C3CCN(C)CC3)c2c1 ; Cl]

Obtained 0.029 g (95% of yield). ¹H NMR (500 MHz, DMSO-d₆) δ ppm 1.76-1.93 (m, 2H), 2.06 (d, J=14.64 Hz, 2H), 2.69 (tt, J=12.09, 3.47 Hz, 1H), 2.76 (d, J=4.73 Hz, 3H), 2.91-3.04 (m, 2H), 3.44-3.52 (m, 2H), 3.67 (s, 3H), 7.10 (d, J=8.39 Hz, 1H), 7.12-7.18 (m, 2H), 7.34-7.41 (m, 1H), 7.47 (d, J=8.54 Hz, 1H), 7.59 (dd, J=1.37, 0.76 Hz, 1H), 9.61 (br. s., 1H), 10.53 (s, 1H), 12.76 (d, J=6.41 Hz, 1H). HRMS (ESI) calcd for C21H24ClN4O2 [M+H]⁺ 399.1583, found 399.1582.

### N-[5-(2,3-Difluorophenyl)-1H-indazol-3-yl]-1-methylpiperidine-4-carboxamide hydrochloride (cpd 99) [(I), R1=2,3-difluorophenyl, R2=1-methylpiperidine, R3=H]

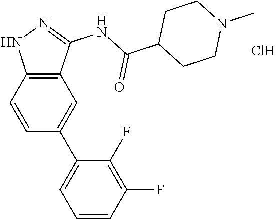 [SMILES: CN1CCC(C(=O)Nc2n[nH]c3ccc(-c4cccc(F)c4F)cc23)CC1 ; Cl]

Obtained 0.039 g (93% of yield). ¹H NMR (500 MHz, DMSO-d₆) δ ppm 1.82-1.96 (m, 2H), 2.06-2.18 (m, 2H), 2.68-2.75 (m, 1H), 2.77 (d, J=4.58 Hz, 3H), 2.90-3.04 (m, 2H), 3.48 (d, J=12.35 Hz, 2H), 7.27-7.38 (m, 2H), 7.39-7.47 (m, 1H), 7.51-7.59 (m, 2H), 7.95-8.02 (m, 1H), 9.64 (br. s., 1H), 10.59-10.67 (m, 1H), 12.89 (br. s., 1H). HRMS (ESI) calcd for C20H21F2N4O [M+H]⁺ 371.1678, found 371.1687.

### 1-Methyl-N-{5-[3-(methylsulfonyl)phenyl]-1H-indazol-3-yl}piperidine-4-carboxamide hydrochloride (cpd 100) [(1), R1=3-(methylsulfonyl)phenyl, R2=1-methylpiperidine, R3=H]

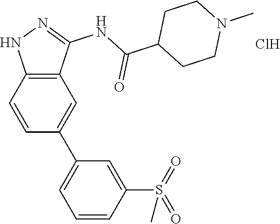 [SMILES: CN1CCC(C(=O)Nc2n[nH]c3ccc(-c4cccc(S(C)(=O)=O)c4)cc23)CC1 ; Cl]

Obtained 0.025 g (88% of yield). ¹H NMR (500 MHz, DMSO-d₆) δ ppm 1.81-1.99 (m, 2H), 2.12 (m, J=13.57 Hz, 2H), 2.65-2.82 (m, 4H), 2.95-3.08 (m, 2H), 3.30 (s, 3H), 3.50 (d, J=10.07 Hz, 2H), 7.59 (d, J=8.69 Hz, 1H), 7.69-7.80 (m, 2H), 7.90 (dd, J=7.24, 1.14 Hz, 1H), 7.99 (d, J=7.93 Hz, 1H), 8.10 (s, 1H), 8.12 (s, 1H), 9.67 (br. s., 1H), 10.22-11.04 (m, 1H), 12.88 (br. s., 1H). HRMS (ESI) calcd for C21H25N4O3S [M+H]⁺ 413.1642, found 413.1653.

### N-[5-(3-Fluorophenyl)-1H-indazol-3-yl]-1-methylpiperidine-4-carboxamide hydrochloride (cpd 101) [(I), R1=3-fluorophenyl, R2=1-methylpiperidine, R3=H]

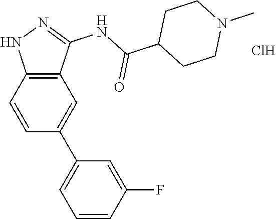 [SMILES: CN1CCC(C(=O)Nc2n[nH]c3ccc(-c4cccc(F)c4)cc23)CC1 ; Cl]

Obtained 0.031 g (89% of yield). ¹H NMR (500 MHz, DMSO-d₆) δ ppm 1.86-1.98 (m, 2H), 2.12 (m, J=14.18 Hz, 2H), 2.73 (m, J=3.97 Hz, 1H), 2.77-2.81 (m, 3H), 2.94-3.05 (m, 2H), 3.50 (m, J=12.51 Hz, 2H), 7.14-7.22 (m, 1H), 7.41-7.57 (m, 4H), 7.64-7.72 (m, 1H), 8.01-8.12 (m, 1H), 9.68 (br. s., 1H) 10.48-10.79 (m, 1H), 12.82 (br. s., 1H). HRMS (ESI) calcd for C20H20Cl2N3O [M+H]⁺ 388.0978, found 388.0981.

### N-[5-(2-Hydroxyphenyl)-1H-indazol-3-yl]-1-methylpiperidine-4-carboxamide hydrochloride (cpd 102) [(I), R1=2-hydroxyphenyl, R2=1-methylpiperidine, R3=H]

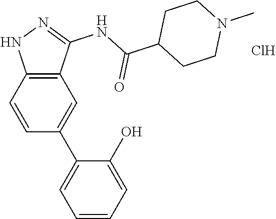 [SMILES: CN1CCC(C(=O)Nc2n[nH]c3ccc(-c4ccccc4O)cc23)CC1 ; Cl]

Obtained 0.030 g (95% of yield). ¹H NMR (500 MHz, DMSO-d₆) δ ppm 1.83-1.95 (m, 2H), 2.05-2.13 (m, 2H), 2.68-2.73 (m, 1H), 2.77 (d, J=4.73 Hz, 3H), 2.92-3.04 (m, 2H), 3.46-3.51 (m, 2H), 6.88 (td, J=7.44, 0.99 Hz, 1H), 6.94 (d, J=8.08 Hz, 1H), 7.15 (td, J=7.70, 1.68 Hz, 1H), 7.18-7.27 (m, 1H), 7.28-7.33 (m, 1H), 7.44 (d, J=8.85 Hz, 1H), 7.53 (dd, J=8.69, 1.22 Hz, 1H), 7.78-7.86 (m, 1H), 9.45 (br. s., 1H), 9.66 (br. s., 1H), 10.45-10.54 (m, 1H), 12.69 (br. s., 1H). HRMS (ESI) calcd for C20H23N4O2 [M+H]⁺ 351.1816, found 351.1811.

### N-[5-(Biphenyl-2-yl)-1H-indazol-3-yl]-1-methylpiperidine-4-carboxamide hydrochloride (cpd 103) [(I), R1=biphenyl-2-yl, R2=1-methylpiperidine, R3=H]

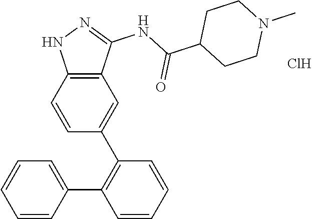 [SMILES: CN1CCC(C(=O)Nc2n[nH]c3ccc(-c4ccccc4-c4ccccc4)cc23)CC1 ; Cl]

Obtained 0.038 g (95% of yield). ¹H NMR (500 MHz, DMSO-d₆) δ ppm 1.79-1.93 (m, 2H), 1.97-2.13 (m, 2H), 2.64-2.72 (m, 1H), 2.74-2.82 (m, 3H), 2.92-3.05 (m, 2H), 3.47-3.53 (m, 2H), 6.84-6.98 (m, 1H), 7.06-7.13 (m, 2H), 7.15-7.27 (m, 4H), 7.38-7.51 (m, 4H), 7.60-7.67 (m, 1H), 9.71 (br. s., 1H), 10.47 (s, 1H), 12.65 (br. s., 1H). HRMS (ESI) calcd for C26H27N4O [M+H]⁺ 411.2180, found 411.2183.

### 1-Methyl-N-{5-[2-(trifluoromethyl)phenyl]-1H-indazol-3-yl}piperidine-4-carboxamide hydrochloride (cpd 104) [(I), R1=2-(trifluoromethyl)phenyl, R2=1-methylpiperidine, R3=H]

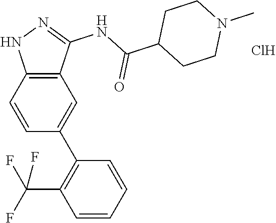 [SMILES: CN1CCC(C(=O)Nc2n[nH]c3ccc(-c4ccccc4C(F)(F)F)cc23)CC1 ; Cl]

Obtained 0.039 g (99% of yield). ¹H NMR (500 MHz, DMSO-d₆) δ ppm 1.81-1.92 (m, 2H), 2.00-2.15 (m, 2H), 2.65-2.73 (m, 1H), 2.75 (d, J=4.58 Hz, 3H), 2.85-3.03 (m, 2H), 7.28 (d, J=8.69 Hz, 1H), 7.43 (d, J=7.47 Hz, 1H), 7.47-7.51 (m, 1H), 7.59-7.65 (m, 1H), 7.68-7.76 (m, 2H), 7.84 (d, J=7.78 Hz, 1H), 9.68 (br. s., 1H), 10.58 (s, 1H), 12.85 (br. s., 1H). HRMS (ESI) calcd for C21H22F3N4O [M+H]⁺ 403.1740, found 403.1736.

### N-[5-(2,6-Difluorophenyl)-1H-indazol-3-yl]-1-methylpiperidine-4-carboxamide hydrochloride (cpd 105) [(I), R1=2,6-difluorophenyl, R2=1-methylpiperidine, R3=H]

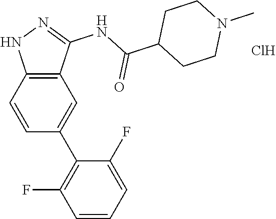 [SMILES: CN1CCC(C(=O)Nc2n[nH]c3ccc(-c4c(F)cccc4F)cc23)CC1 ; Cl]

Obtained 0.022 g (98% of yield). ¹H NMR (500 MHz, DMSO-d₆) δ ppm 1.82-1.94 (m, 2H), 2.04-2.13 (m, 2H), 2.71 (t, J=12.12 Hz, 1H), 2.75-2.79 (m, 2H), 2.93-3.05 (m, 2H), 3.47 (d, J=10.98 Hz, 3H), 7.16-7.27 (m, 2H), 7.38 (d, J=8.85 Hz, 1H), 7.42-7.52 (m, 1H), 7.56 (d, J=8.69 Hz, 1H), 7.86 (s, 1H), 9.64 (br. s., 1H), 10.59-10.64 (m, 1H), 12.89 (br. s., 1H). HRMS (ESI) calcd for C20H21F2N4O [M+H]⁺ 371.1678, found 371.1679.

### N-[5-(2,4-Dichlorophenyl)-1H-indazol-3-yl]-1-methylpiperidine-4-carboxamide hydrochloride (cpd 106) [(I), R1=2,4-dichlorophenyl, R2=1-methylpiperidine, R3=H]

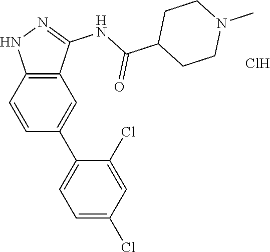 [SMILES: CN1CCC(C(=O)Nc2n[nH]c3ccc(-c4ccc(Cl)cc4Cl)cc23)CC1 ; Cl]

After completion of the reaction, the crude was purified by flash column chromatography (DCM/MeOH/33% aqueous ammonia 90:10:01) to obtain 0.028 g (75% of yield). ¹H NMR (500 MHz, DMSO-d₆) δ ppm 1.61-1.72 (m, 2H), 1.79 (d, J=11.59 Hz, 2H), 1.88 (t, J=10.75 Hz, 2H), 2.16 (s, 3H), 2.41 (br. s., 1H), 2.81 (d, J=11.29 Hz, 2H), 7.37 (dd, J=8.62, 1.60 Hz, 1H), 7.43-7.46 (m, 1H), 7.48-7.55 (m, 2H), 7.73 (d, J=2.14 Hz, 1H), 7.78-7.80 (m, 1H), 10.36 (s, 1H), 12.78 (br. s., 1H). HRMS (ESI) calcd for C20H21Cl2N4O [M+H]⁺ 403.1087, found 403.1087.

### N-[5-(4-Amino-2-chlorophenyl)-1H-indazol-3-yl]-1-methylpiperidine-4-carboxamide dihydrochloride (cpd 107) [(I), R1=4-amino-2-chlorophenyl, R2=1-methylpiperidine, R3=H]

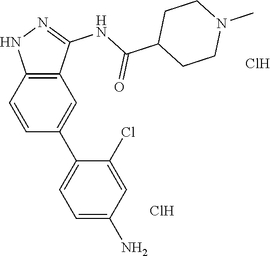 [SMILES: CN1CCC(C(=O)Nc2n[nH]c3ccc(-c4ccc(N)cc4Cl)cc23)CC1 ; Cl ; Cl]

Obtained 0.055 g (99% of yield). ¹H NMR (500 MHz, DMSO-d₆) δ ppm 1.82-1.95 (m, 2H), 2.03-2.11 (m, 2H), 2.66-2.73 (m, 1H), 2.77 (d, J=4.73 Hz, 3H), 2.93-3.04 (m, 2H), 3.14-3.25 (m, 2H), 6.83 (d, J=6.71 Hz, 1H), 6.96 (br. s., 1H), 7.18-7.20 (m, 1H), 7.33-7.38 (m, 1H), 7.46 (d, J=8.69 Hz, 1H), 7.68-7.76 (m, 1H), 9.77 (br. s., 1H), 10.50-10.58 (m, 1H), 12.58-13.12 (m, 1H). HRMS (ESI) calcd for C20H23ClN5O [M+H]⁺ 384.1586, found 384.1582.

### 1-Methyl-N-[5-(2,4,6-trichlorophenyl)-1H-indazol-3-yl]piperidine-4-carboxamide hydrochloride (cpd 108) [(I), R1=2,4,6-trichlorophenyl, R2=1-methylpiperidine, R3=H]

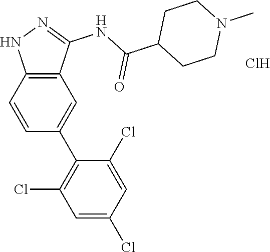 [SMILES: CN1CCC(C(=O)Nc2n[nH]c3ccc(-c4c(Cl)cc(Cl)cc4Cl)cc23)CC1 ; Cl]

Obtained 0.021 g (79% of yield). ¹H NMR (500 MHz, DMSO-d₆) δ ppm 1.78-2.01 (m, 2H), 2.04-2.17 (m, 2H), 2.70 (t, J=11.97 Hz, 1H), 2.76 (d, J=4.88 Hz, 3H), 2.88-3.02 (m, 2H), 3.47 (d, J=11.44 Hz, 2H), 7.19 (dd, J=8.69, 1.53 Hz, 1H), 7.55 (d, J=8.69 Hz, 1H), 7.76 (s, 1H), 7.81 (s, 2H), 9.63 (br. s., 1H), 10.52-10.66 (m, 1H), 12.89 (br. s., 1H). HRMS (ESI) calcd for C20H2OC13N4O [M+H]⁺ 437.0697, found 437.0691.

### N-[5-(4-Carbamoylphenyl)-1H-indazol-3-yl]-1-methylpiperidine-4-carboxamide hydrochloride (cpd 109) [(I), R1=4-carbamoylphenyl, R2=1-methylpiperidine, R3=H]

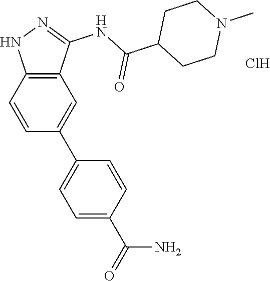 [SMILES: CN1CCC(C(=O)Nc2n[nH]c3ccc(-c4ccc(C(N)=O)cc4)cc23)CC1 ; Cl]

Obtained 0.032 g (92% of yield). 1H NMR (500 MHz, DMSO-d₆) δ ppm 1.86-2.06 (m, 2H), 2.12 (d, J=13.57 Hz, 2H), 2.69-2.76 (m, 1H), 2.76-2.81 (m, 3H), 2.92-3.06 (m, 2H), 3.50 (d, J=11.90 Hz, 2H), 7.39 (br. s., 1H), 7.55 (d, J=8.69 Hz, 1H), 7.69-7.75 (m, 3H), 7.97 (d, J=8.54 Hz, 2H), 8.02 (br. s., 1H), 8.06-8.13 (m, 1H), 9.69 (br. s., 1H), 10.56-10.64 (m, 1H), 12.95 (br. s., 1H). HRMS (ESI) calcd for C21H24N5O2 [M+H]⁺ 378.1925, found 378.1923.

### N-[5-(4-Cyanophenyl)-1H-indazol-3-yl]-1-methylpiperidine-4-carboxamide hydrochloride (cpd 110) [(I), R1=4-cyanophenyl, R2=1-methylpiperidine, R3=H]

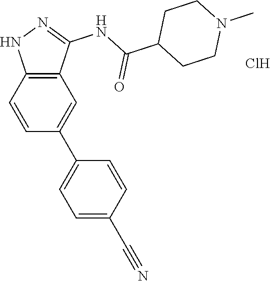 [SMILES: CN1CCC(C(=O)Nc2n[nH]c3ccc(-c4ccc(C#N)cc4)cc23)CC1 ; Cl]

Obtained 0.027 g (88% of yield). ¹H NMR (500 MHz, DMSO-d₆) δ ppm 1.87-2.00 (m, 2H), 2.11 (d, J=13.42 Hz, 2H), 2.69-2.76 (m, 1H), 2.76-2.80 (m, 3H), 2.90-3.06 (m, 2H), 7.57 (d, J=8.69 Hz, 1H), 7.73 (dd, J=8.77, 1.45 Hz, 1H), 7.84 (d, J=6.86 Hz, 2H), 7.94 (d, J=6.86 Hz, 2H), 8.14 (s, 1H), 9.79 (br. s., 1H), 10.59-10.75 (m, 1H), 12.90 (br. s., 1H). HRMS (ESI) calcd for C21H22N5O [M+H]⁺ 360.1819, found 360.1814.

### N-[5-(2-Chloro-4-hydroxyphenyl)-1H-indazol-3-yl]-1-methylpiperidine-4-carboxamide hydrochloride (cpd 111) [(I), R1=2-chloro-4-hydroxyphenyl, R2=1-methylpiperidine, R3=H]

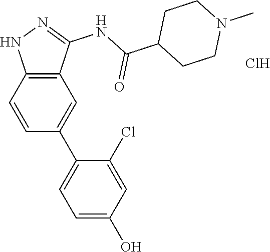 [SMILES: CN1CCC(C(=O)Nc2n[nH]c3ccc(-c4ccc(O)cc4Cl)cc23)CC1 ; Cl]

Obtained 0.029 g (75% of yield). ¹H NMR (500 MHz, DMSO-d₆) δ ppm 1.83-1.92 (m, 2H), 2.04-2.09 (m, 2H), 2.68-2.73 (m, 1H), 2.76 (d, J=4.58 Hz, 3H), 2.94-2.99 (m, 2H), 6.83 (dd, J=8.24, 2.44 Hz, 1H), 6.94 (d, J=2.44 Hz, 1H), 7.45-7.49 (m, 2H), 7.34-7.35 (m, 1H), 7.70 (dd, J=1.53, 0.76 Hz, 1H), 9.65 (br. s., 1H), 9.98 (br. s., 1H), 10.36-10.76 (m, 1H), 12.77 (br. s., 1H). HRMS (ESI) calcd for C20H22ClN4O2 [M+H]⁺ 385.1426, found 385.1427.

### N-[5-(4-Amino-3-fluorophenyl)-1H-indazol-3-yl]-1-methylpiperidine-4-carboxamide hydrochloride (cpd 112) [(I), R1=4-amino-3-fluorophenyl, R2=1-methylpiperidine, R3=H]

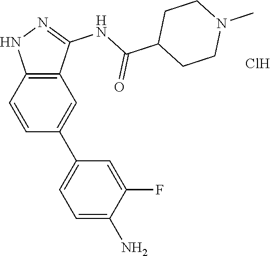 [SMILES: CN1CCC(C(=O)Nc2n[nH]c3ccc(-c4ccc(N)c(F)c4)cc23)CC1 ; Cl]

Obtained 0.031 g (99% of yield). 1H NMR (500 MHz, DMSO-d₆) δ ppm 1.74-1.83 (m, 2H), 1.89-1.99 (m, 2H), 2.02-2.14 (m, 2H), 2.70-2.78 (m, 4H), 2.91-3.03 (m, 2H), 6.95-6.03 (m, 1H), 7.25-7.38 (m, 1H), 7.44-7.49 (m, 1H), 7.54-7.86 (m, 1H), 7.83 (dd, J=9.00, 1.37 Hz, 1H), 7.89 (s, 1H), 9.87 (br. s., 2H), 10.54 (s, 1H), 12.77 (br. s., 1H). HRMS (ESI) calcd for C20H23FN5O [M+H]⁺ 368.1881, found 368.1882.

### N-[5-(4-Cyano-2-methylphenyl)-1H-indazol-3-yl]-1-methylpiperidine-4-carboxamide hydrochloride (cpd 113) [(I), R1=4-cyano-2-methylphenyl, R2=1-methylpiperidine, R3=H]

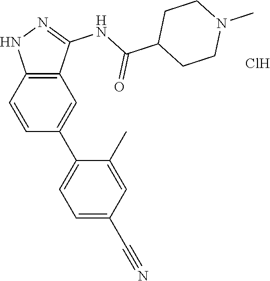 [SMILES: Cc1cc(C#N)ccc1-c1ccc2[nH]nc(NC(=O)C3CCN(C)CC3)c2c1 ; Cl]

Obtained 0.024 g (94% of yield). 1H NMR (500 MHz, DMSO-d₆) δ ppm 1.83-1.92 (m, 2H), 2.01-2.17 (m, 2H), 2.28 (s, 3H), 2.70 (m, 1H), 2.76 (d, J=4.58 Hz, 3H), 2.89-3.02 (m, 2H), 3.45-3.50 (m, 2H), 7.36 (dd, J=8.62, 1.60 Hz, 1H), 7.41 (d, J=7.93 Hz, 1H), 7.53 (d, J=8.69 Hz, 1H), 7.70-7.77 (m, 2H), 7.81 (d, J=1.22 Hz, 1H), 9.70 (br. s., 1H), 10.51-10.69 (m, 1H), 12.86 (br. s., 1H). HRMS (ESI) calcd for C22H24N5O [M+H]⁺ 374.1976, found 374.1976.

### N-[5-(2,6-Dimethylphenyl)-1H-indazol-3-yl]-1-methylpiperidine-4-carboxamide trifluoroacetate (cpd 114) [(I), R1=2,6-dimethylphenyl, R2=1-methylpiperidine, R3=H]

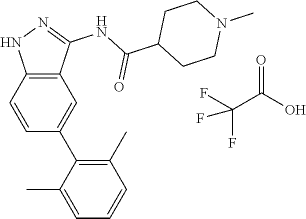

Obtained 0.066 g (90% of yield). ¹H NMR (500 MHz, DMSO-d₆) δ ppm 1.75-1.88 (m, 2H), 1.97 (s, 6H), 2.03-2.17 (m, 2H), 2.68 (m, 1H), 2.77 (d, J=4.58 Hz, 3H), 2.91-3.03 (m, 2H), 3.42-3.49 (m, 2H), 7.05-7.20 (m, 5H), 7.40-7.58 (m, 2H), 9.19 (br. s., 1H), 10.52 (s, 1H), 12.75 (br. s., 1H). HRMS (ESI) calcd for C22H27N4O [M+H]⁺ 363.2180, found 363.2176.

### N-[5-(2-Fluoro-5-nitrophenyl)-1H-indazol-3-yl]-1-methylpiperidine-4-carboxamide hydrochloride (cpd 115) [(I), R1=2-fluoro-5-nitrophenyl, R2=1-methylpiperidine, R3=H]

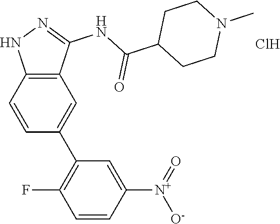 [SMILES: CN1CCC(C(=O)Nc2n[nH]c3ccc(-c4cc([N+](=O)[O-])ccc4F)cc23)CC1 ; Cl]

Obtained 0.047 g (98% of yield). ¹H NMR (500 MHz, DMSO-d₆) δ ppm 1.83-1.97 (m, 2H), 2.04-2.14 (m, 2H), 2.68-2.75 (m, 1H), 2.77 (d, J=4.88 Hz, 3H), 2.91-3.02 (m, 2H), 3.45-3.52 (m, 2H), 7.60-7.63 (m, 2H), 7.65 (t, J=9.38 Hz, 1H), 8.06 (s, 1H), 8.26-8.36 (m, 2H), 9.70 (br. s., 1H), 10.63-10.70 (m, 1H), 12.95 (br. s., 1H). HRMS (ESI) calcd for C20H21FN5O3 [M+H]⁺ 398.1623, found 398.1620.

### N-[5-(2-Chloro-5-cyanophenyl)-1H-indazol-3-yl]-1-methylpiperidine-4-carboxamide trifluoroacetate (cpd 116) [(I), R1=2-chloro-5-cyanophenyl, R2=1-methylpiperidine, R3=H]

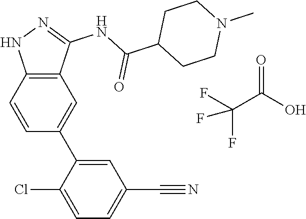

Obtained 0.035 g (91% of yield). ¹H NMR (500 MHz, DMSO-d₆) δ ppm 1.77-1.90 (m, 2H), 2.06-2.15 (m, 2H), 2.71 (m, 1H), 2.79 (br. s., 3H), 2.95-3.04 (m, 2H), 3.48-351 (m, 2H), 7.42 (dd, J=8.85, 1.22 Hz, 1H), 7.55 (d, J=8.54 Hz, 1H), 7.81 (d, J=8.24 Hz, 1H), 7.85 (s, 1H) 7.89 (dd, J=8.24, 1.98 Hz, 1H), 7.93 (d, J=1.98 Hz, 1H), 9.20 (br. s., 1H), 10.58-10.66 (m, 1H), 12.89 (br.s., 1H). HRMS (ESI) calcd for C21H21ClN5O [M+H]⁺ 394.1429, found 394.1435.

### N-[5-(5-Carbamoyl-2-chlorophenyl)-1H-indazol-3-yl]-1-methylpiperidine-4-carboxamide trifluoroacetate (cpd 117) [(I), R1=5-carbamoyl-2-chlorophenyl, R2=1-methylpiperidine, R3=H]

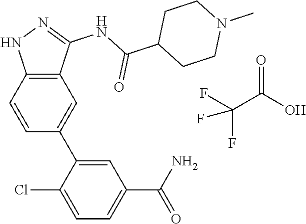

Obtained 0.058 g (77% of yield). ¹H NMR (500 MHz, DMSO-d₆) δ ppm 1.77-1.91 (m, 2H), 2.06-2.18 (m, 2H), 2.70 (m, 1H), 2.79 (d, J=4.42 Hz, 3H), 2.94-3.03 (m, 2H), 3.49 (d, J=11.74 Hz, 2H), 7.43 (dd, J=8.54, 1.22 Hz, 1H), 7.50 (s, 1H), 7.54 (d, J=8.54 Hz, 1H), 7.82 (s, 1H), 7.88 (dd, J=8.39, 2.14 Hz, 1H), 7.92 (d, J=1.98 Hz, 1H), 8.12 (s, 1H), 9.20 (br. s., 1H), 10.61 (s, 1H), 12.86 (s, 1H). HRMS (ESI) calcd for C21H23ClN5O2 [M+H]⁺ 412.1535, found 412.1537.

### N-[5-(2-Fluoro-4-formylphenyl)-1H-indazol-3-yl]-1-methylpiperidine-4-carboxamide hydrochloride (cpd 118) [(I), R1=2-fluoro-4-formylphenyl, R2=1-methylpiperidine, R3=H]

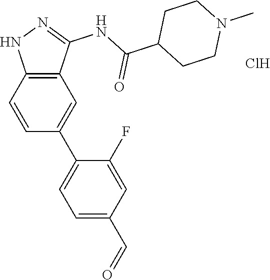 [SMILES: CN1CCC(C(=O)Nc2n[nH]c3ccc(-c4ccc(C=O)cc4F)cc23)CC1 ; Cl]

Obtained 0.024 g (90% of yield). ¹H NMR (500 MHz, DMSO-d₆) δ ppm 1.85-1.98 (m, 2H), 2.04-2.15 (m, 2H), 2.73 (m, 1H), 2.77 (d, J=4.58 Hz, 3H), 2.94-3.04 (m, 2H), 3.49 (d, J=11.29 Hz, 2H), 7.57-7.60 (m, 2H), 7.79 (m, 1H), 7.83 (d, J=10.68 Hz, 1H), 7.88 (dd, J=7.78, 1.37 Hz, 1H), 8.06 (s, 1H), 9.63 (br. s., 1H), 10.05 (s, 1H), 10.66 (s, 1H), 12.92 (br. s., 1H). HRMS (ESI) calcd for C21H22FN4O2 [M+H]⁺ 381.1722, found 378.1718.

### N-[5-(2-Fluoro-4-methylphenyl)-1H-indazol-3-yl]-1-methylpiperidine-4-carboxamide hydrochloride (cpd 119) [(I), R1=2-fluoro-4-methylphenyl, R2=1-methylpiperidine, R3=H]

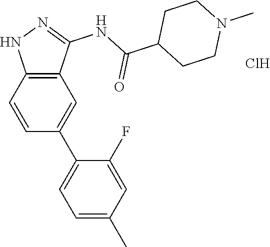 [SMILES: Cc1ccc(-c2ccc3[nH]nc(NC(=O)C4CCN(C)CC4)c3c2)c(F)c1 ; Cl]

Obtained 0.022 g (68% of yield). ¹H NMR (500 MHz, DMSO-d₆) δ ppm 1.82-1.95 (m, 2H), 2.06-2.15 (m, 2H), 2.36 (s, 3H), 2.72 (m, 1H), 2.77 (d, J=4.88 Hz, 2H), 2.94-3.06 (m, 1H), 3.48 (d, J=12.51 Hz, 2H), 7.09-7.16 (m, 2H), 7.39 (m, 1H), 7.46-7.55 (m, 2H), 7.88 (s, 1H), 9.68 (br. s., 1H), 10.58 (s, 1H), 12.81 (br. s., 1H). HRMS (ESI) calcd for C21H24FN4O [M+H]⁺ 367.1929, found 367.1922.

### N-[5-(2,6-Difluoro-4-methoxyphenyl)-1H-indazol-3-yl]-1-methylpiperidine-4-carboxamide hydrochloride (cpd 120) [(I), R1=2,6-difluoro-4-methoxyphenyl, R2=1-methylpiperidine, R3=H]

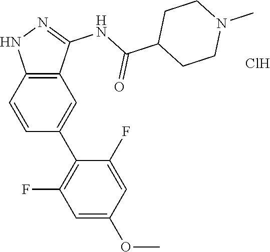 [SMILES: COc1cc(F)c(-c2ccc3[nH]nc(NC(=O)C4CCN(C)CC4)c3c2)c(F)c1 ; Cl]

Obtained 0.013 g (75% of yield). ¹H NMR (500 MHz, DMSO-d₆) δ ppm 1.83-1.97 (m, 2H), 2.05-2.13 (m, 2H), 2.71 (m, 1H), 2.77 (d, J=4.73 Hz, 3H), 2.94-3.04 (m, 2H), 3.48 (d, J=10.68 Hz, 2H), 3.83 (s, 3H), 6.82-6.89 (m, 2H), 7.33 (d, J=8.54 Hz, 1H), 7.53 (d, J=8.69 Hz, 1H), 7.79 (s, 1H), 9.60 (br. s., 1H), 10.59 (s, 1H), 12.85 (br. s., 1H). HRMS (ESI) calcd for C21H23F2N4O2 [M+H]401.1784, found 401.1778.

### 1-Methyl-N-[5-(1H-pyrazol-4-yl)-1H-indazol-3-yl]piperidine-4-carboxamide hydrochloride (cpd 121) [(I), R1=1H-pyrazol-4-yl, R2=1-methylpiperidine, R3=H]

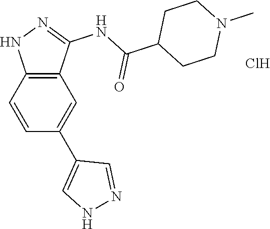 [SMILES: CN1CCC(C(=O)Nc2n[nH]c3ccc(-c4cn[nH]c4)cc23)CC1 ; Cl]

Obtained 0.036 g (88% of yield). 1H NMR (500 MHz, DMSO-d₆) δ ppm 1.83-2.00 (m, 2H), 2.08-2.14 (m, 2H), 2.75-2.81 (m, 4H), 2.90-3.06 (m, 2H), 7.44 (d, J=8.54 Hz, 1H), 7.60 (dd, J=8.62, 1.14 Hz, 1H), 7.83 (s, 1H), 7.96-8.01 (m, 2H), 9.72 (br. s., 1H), 10.44 (s, 1H), 12.69 (br. s., 1H). HRMS (ESI) calcd for C17H21N6O [M+H]⁺ 325.1772, found 325.1772.

### 1-Methyl-N-[5-(1-methyl-1H-pyrazol-4-yl)-1H-indazol-3-yl]piperidine-4-carboxamide hydrochloride (cpd 122) [(I), R1=1-methyl-1H-pyrazol-4-yl, R2=1-methylpiperidine, R3=H]

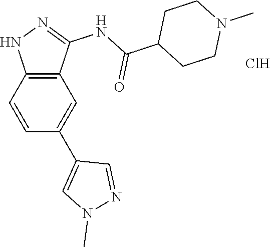 [SMILES: CN1CCC(C(=O)Nc2n[nH]c3ccc(-c4cnn(C)c4)cc23)CC1 ; Cl]

Obtained 0.045 g (87% of yield). 1H NMR (500 MHz, DMSO-d₆) δ ppm 1.85-2.02 (m, 2H), 2.04-2.16 (m, 2H), 2.67-2.75 (m, 1H), 2.77 d, J=4.73 Hz, 3H), 2.93-3.07 (m, 2H), 3.87 (s, 3H), 7.43 (d, J=8.69 Hz, 1H), 7.55 (dd, J=8.69, 1.37 Hz, 1H), 7.74-7.84 (m, 2H), 8.05 (s, 1H), 9.85 (br. s., 1H), 10.46 (s, 1H), 12.69 (br. s., 1H). HRMS (ESI) calcd for C18H23N6O [M+H]⁺ 339.1928, found 339.1926.

### 1-Methyl-N-[5-(2,4,6-trifluorophenyl)-1H-indazol-3-yl]piperidine-4-carboxamide hydrochloride (cpd 123) [(I), R1=2,4,6-trifluorophenyl, R2=1-methylpiperidine, R3=H]

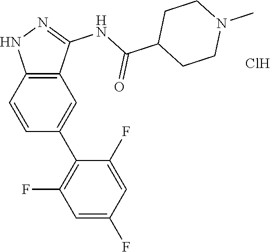 [SMILES: CN1CCC(C(=O)Nc2n[nH]c3ccc(-c4c(F)cc(F)cc4F)cc23)CC1 ; Cl]

Obtained 0.005 g (71% of yield). ¹H NMR (500 MHz, DMSO-d₆) δ ppm 1.80-1.96 (m, 2H), 2.05-2.13 (m, 2H), 2.71 (m, 1H), 2.77 (d, J=4.73 Hz, 3H), 2.93-3.03 (m, 2H), 3.48 (d, J=10.83 Hz, 2H), 7.27-7.41 (m, 3H), 7.56 (d, J=8.54 Hz, 1H), 7.84 (s, 1H), 9.59 (br. s., 1H), 10.62 (s, 1H), 12.90 (br. s., 1H). HRMS (ESI) calcd for C20H20F3N4O [M+H]⁺ 389.1584, found 389.1585.

### N-[5-(2,4-Difluorophenyl)-1H-indazol-3-yl]-1-methylpiperidine-4-carboxamide hydrochloride (cpd 124) [(I), R1=2,4-difluorophenyl, R2=1-methylpiperidine, R3=H]

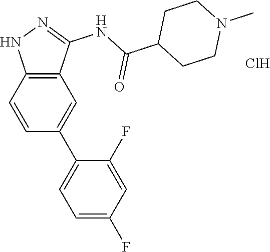 [SMILES: CN1CCC(C(=O)Nc2n[nH]c3ccc(-c4ccc(F)cc4F)cc23)CC1 ; Cl]

Obtained 0.037 g (84% of yield). ¹H NMR (500 MHz, DMSO-d₆) δ ppm 1.83-1.96 (m, 2H), 2.05-2.10 (m, 2H), 2.72 (m, 1H), 2.77 (d, J=4.58 Hz, 3H), 2.89-3.05 (m, 2H), 3.48 (d, J=12.96 Hz, 2H), 7.21 (td, J=8.50, 2.52 Hz, 1H), 7.37 (ddd, J=11.17, 9.19, 2.52 Hz, 1H), 7.45-7.49 (m, 1H), 7.54-7.60 (m, 2H), 7.89 (s, 1H), 9.66 (br. s., 1H), 10.61 (s, 1H), 12.85 (br. s., 1H). HRMS (ESI) calcd for C20H21F2N4O [M+H]⁺ 371.1678, found 371.1677.

### 1-Methyl-N-[5-(1H-pyrazol-3-yl)-1H-indazol-3-yl]piperidine-4-carboxamide hydrochloride (cpd 125) [(I), R1=1H-pyrazol-3-yl, R2=1-methylpiperidine, R3=H]

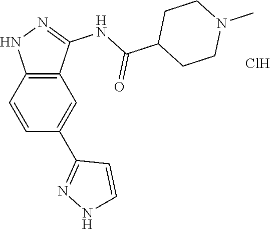 [SMILES: CN1CCC(C(=O)Nc2n[nH]c3ccc(-c4cc[nH]n4)cc23)CC1 ; Cl]

Obtained 0.022 g (88% of yield). ¹H NMR (500 MHz, DMSO-d₆) δ ppm 1.84-2.07 (m, 2H), 2.12 (d, J=13.57 Hz, 2H), 2.73 (m, 1H), 2.78 (t, J=11.97 Hz, 3H), 2.95-3.08 (m, 2H), 6.60-6.66 (m, 1H), 7.47 (d, J=8.85 Hz, 1H), 7.72 (s, 1H), 7.80 (d, J=8.69 Hz, 1H), 8.11 (s, 1H), 9.72 (br. s., 1H), 10.50 (s, 1H), 12.75 (br. s., 1H). HRMS (ESI) calcd for C17H21N6O [M+H]⁺ 325.1772, found 328.1769.

### 1-Methyl-N-[5-(2,4,5-trifluorophenyl)-1H-indazol-3-yl]piperidine-4-carboxamide hydrochloride (cpd 126) [(I), R1=2,4,5-trifluorophenyl, R2=1-methylpiperidine, R3=H]

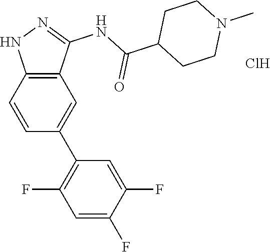 [SMILES: CN1CCC(C(=O)Nc2n[nH]c3ccc(-c4cc(F)c(F)cc4F)cc23)CC1 ; Cl]

Obtained 0.030 g (93% of yield). ¹H NMR (500 MHz, DMSO-d₆) δ ppm 1.81-2.04 (m, 2H), 2.04-2.23 (m, 2H), 2.72 (m, 1H), 2.77 (d, J=4.58 Hz, 3H), 2.86-3.07 (m, 2H), 3.49 (d, J=12.51 Hz, 2H), 7.49 (d, J=8.39 Hz, 1H), 7.54 (d, J=8.69 Hz, 1H), 7.60-7.73 (m, 2H), 7.93 (s, 1H), 9.65 (br. s., 1H), 10.62 (s, 1H), 12.88 (br. s., 1H). HRMS (ESI) calcd for C20H20F3N4O [M+H]⁺ 389.1584, found 389.1590.

### 1-Methyl-N-{5-[2,4,6-trifluoro-3-(propan-2-yloxy)phenyl]-1H-indazol-3-yl}piperidine-4-carboxamide hydrochloride (cpd 127) [(I), R1=2,4,6-trifluoro-3-(propan-2-yloxy)phenyl, R2=1-methylpiperidine, R3=H]

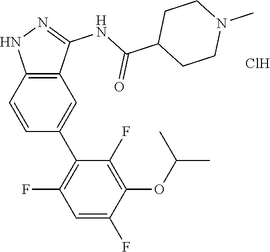 [SMILES: CC(C)Oc1c(F)cc(F)c(-c2ccc3[nH]nc(NC(=O)C4CCN(C)CC4)c3c2)c1F ; Cl]

Obtained 0.044 g (96% of yield). ¹H NMR (500 MHz, DMSO-d₆) δ ppm 1.29 (d, J=6.10 Hz, 6H), 1.77-2.04 (m, 2H), 2.04-2.21 (m, 2H), 2.71 (m, 1H), 2.77 (d, J=4.88 Hz, 3H), 2.88-3.05 (m, 2H), 3.48 (d, J=11.90 Hz, 2H), 4.35 (m, 1H), 7.32-7.45 (m, 2H), 7.56 (d, J=8.69 Hz, 1H), 7.85 (s, 1H), 9.62 (br. s., 1H), 10.62 (s, 1H), 12.90 (br. s., 1H). HRMS (ESI) calcd for C23H26F3N4O2 [M+H]⁺ 447.2003, found 447.20.

### 1-Methyl-N-[5-(2,3,4-trifluorophenyl)-1H-indazol-3-yl]piperidine-4-carboxamide hydrochloride (cpd 128) [(I), R1=2,3,4-trifluorophenyl, R2=1-methylpiperidine, R3=H]

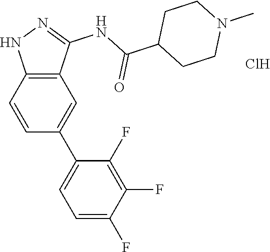 [SMILES: CN1CCC(C(=O)Nc2n[nH]c3ccc(-c4ccc(F)c(F)c4F)cc23)CC1 ; Cl]

Obtained 0.051 g (97% of yield). ¹H NMR (500 MHz, DMSO-d₆) δ ppm 1.84-1.96 (m, 2H), 2.06-2.19 (m, 2H), 2.72 (m, 1H), 2.77 (d, J=4.58 Hz, 3H), 2.94-3.07 (m, 2H), 3.43-3.54 (m, 2H), 7.34-7.47 (m, 2H), 7.49 (d, J=8.85 Hz, 1H), 7.56 (d, J=8.69 Hz, 1H), 7.91-8.00 (m, 1H), 9.67 (br. s., 1H), 10.64 (s, 1H), 12.90 (br. s., 1H). HRMS (ESI) calcd for C20H20F3N4O [M+H]⁺ 389.1584, found 389.1585.

### N-[5-(4-fluoro-2-methylphenyl)-1H-indazol-3-yl]-1-methylpiperidine-4-carboxamide trifluoroacetate (cpd 129) [(I), R1=4-fluoro-2-methylphenyl, R2=1-methylpiperidine, R3=H]

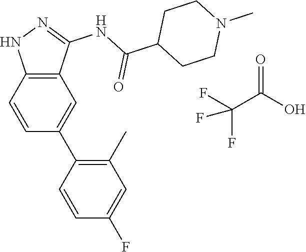

Obtained 0.034 g (88% of yield). 1H NMR (500 MHz, DMSO-d₆) δ ppm 1.77-1.92 (m, 2H), 2.04-2.13 (m, 2H), 2.22 (s, 3H), 2.70 (m, 1H) 2.79 (d, J=4.58 Hz, 3H), 2.93-3.04 (m, 2H), 3.49 (d, J=12.05 Hz, 2H), 7.09 (td, J=8.54, 2.75 Hz, 1H), 7.17 (dd, J=10.14, 2.67 Hz, 1H), 7.23 (dd, J=8.39, 6.25 Hz, 1H), 7.30 (dd, J=8.54, 1.37 Hz, 1H), 7.50 (d, J=8.54 Hz, 1H), 7.64-7.73 (m, 1H), 9.19 (br. s., 1H), 10.56 (s, 1H), 12.77 (br. s., 1H). HRMS (ESI) calcd for C21H24FN4O [M+H]⁺ 367.1929, found 367.1929.

### N-[5-(4-Amino-2-methylphenyl)-1H-indazol-3-yl]-1-methylpiperidine-4-carboxamide dihydrochloride (cpd 130) [(I), R1=4-amino-2-methylphenyl, R2=1-methylpiperidine, R3=H]

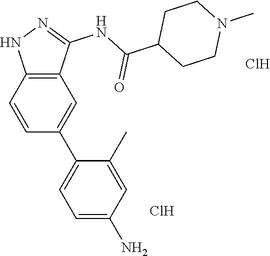 [SMILES: Cc1cc(N)ccc1-c1ccc2[nH]nc(NC(=O)C3CCN(C)CC3)c2c1 ; Cl ; Cl]

Obtained 0.028 g (91% of yield). ¹H NMR (500 MHz, DMSO-d₆) δ ppm 1.85-1.95 (m, 2H), 2.03-2.11 (m, 2H), 2.25 (s, 3H), 2.70 (m, 1H), 2.75 (d, J=4.27 Hz, 3H), 2.92-3.03 (m, 2H), 7.15-7.25 (m, 2H), 7.27-7.34 (m, 2H), 7.50 (d, J=8.69 Hz, 1H), 7.65-7.76 (m, 1H), 9.93 (br. s., 2H), 10.59 (s, 1H), 12.81 (br. s., 1H). HRMS (ESI) calcd for C21H26N5O [M+H]⁺ 364.2132, found 364.2133.

### 1-Methyl-N-[5-(2,4,6-trifluoro-3-methoxyphenyl)-1H-indazol-3-yl]piperidine-4-carboxamide hydrochloride (cpd 131) [(I), R1=2,4,6-trifluoro-3-methoxyphenyl, R2=1-methylpiperidine, R3=H]

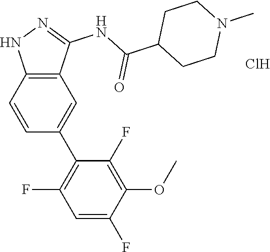 [SMILES: COc1c(F)cc(F)c(-c2ccc3[nH]nc(NC(=O)C4CCN(C)CC4)c3c2)c1F ; Cl]

Obtained 0.037 g (94% of yield). 1H NMR (500 MHz, DMSO-d₆) δ ppm 1.78-1.97 (m, 2H), 2.04-2.18 (m, 2H), 2.72 (m, 1H), 2.77 (d, J=4.58 Hz, 3H), 2.91-3.05 (m, 2H), 3.48 (d, J=12.05 Hz, 2H), 3.93 (s, 3H), 7.32-7.44 (m, 2H), 7.56 (d, J=8.69 Hz, 1H), 7.83-7.91 (m, 1H), 9.66 (br. s., 1H), 10.63 (s, 1H), 12.91 (br. s., 1H). HRMS (ESI) calcd for C21H22F3N4O2 [M+H]⁺ 419.1690, found 419.1698.

### N-[5-(2,3-Difluoro-4-methylphenyl)-1H-indazol-3-yl]-1-methylpiperidine-4-carboxamide hydrochloride (cpd 132) [(I), R1=2,3-difluoro-4-methylphenyl, R2=1-methylpiperidine, R3=H]

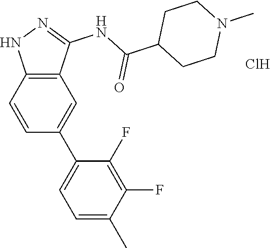 [SMILES: Cc1ccc(-c2ccc3[nH]nc(NC(=O)C4CCN(C)CC4)c3c2)c(F)c1F ; Cl]

Obtained 0.026 g (79% of yield). ¹H NMR (500 MHz, DMSO-d₆) δ ppm 1.83-1.96 (m, 2H), 2.05-2.15 (m, 2H), 2.33 (m, 3H), 2.72 (m, 1H), 2.77 (d, J=4.42 Hz, 3H), 2.92-3.04 (m, 2H), 3.48 (d, J=11.44 Hz, 2H), 7.15-7.27 (m, 2H), 7.49-7.56 (m, 2H), 7.92-7.98 (m, 1H), 9.67 (br. s., 1H), 10.61 (s, 1H), 12.86 (br. s., 1H). HRMS (ESI) calcd for C21H23F2N4O [M+H]⁺ 385.1835, found 385.1835.

### N-[5-(2-Chloro-4-fluoro-3-methylphenyl)-1H-indazol-3-yl]-1-methylpiperidine-4-carboxamide hydrochloride (cpd 133) [(I), R1=2-chloro-4-fluoro-3-methylphenyl, R2=1-methylpiperidine, R3=H]

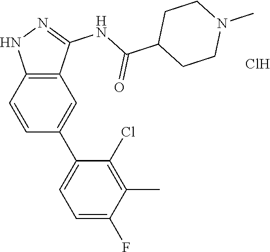 [SMILES: Cc1c(F)ccc(-c2ccc3[nH]nc(NC(=O)C4CCN(C)CC4)c3c2)c1Cl ; Cl]

Obtained 0.029 g (74% of yield). ¹H NMR (500 MHz, DMSO-d₆) δ ppm 1.79-1.93 (m, 2H), 2.02-2.12 (m, 2H), 2.34 (m, 3H), 2.70 (m, 1H), 2.76 (d, J=4.58 Hz, 3H), 2.92-3.05 (m, 2H), 3.47 (d, J=11.29 Hz, 2H), 7.28-7.29 (m, 2H), 7.34 (dd, J=8.62, 1.45 Hz, 1H), 7.50 (d, J=8.85 Hz, 1H), 7.72-7.81 (m, 1H), 9.65 (br. s., 1H), 10.59 (s, 1H), 12.83 (br. s., 1H). HRMS (ESI) calcd for C21H23ClFN4O [M+H]⁺ 401.1539, found 401.1533.

### N-[5-(3-Cyano-2,6-difluorophenyl)-1H-indazol-3-yl]-1-methylpiperidine-4-carboxamide (cpd 134) [(I), R1=3-cyano-2,6-difluorophenyl, R2=1-methylpiperidine, R3=H]

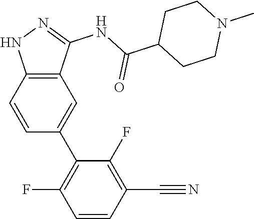 [SMILES: CN1CCC(C(=O)Nc2n[nH]c3ccc(-c4c(F)ccc(C#N)c4F)cc23)CC1]

After completion of the reaction, the crude was purified by flash column chromatography (DCM/MeOH/33% aqueous ammonia 90:10:0.1) to obtain 0.051 g (82% of yield). ¹H NMR (500 MHz, DMSO-d₆) δ ppm 1.61-1.71 (m, 2H), 1.79 (d, J=12.66 Hz, 2H), 1.82-1.91 (m, 2H), 2.15 (s, 3H), 2.40 (m, 1H), 2.80 (d, J=11.29 Hz, 2H), 7.41 (d, J=8.69 Hz, 1H), 7.49 (t, J=8.24 Hz, 1H), 7.58 (d, J=8.69 Hz, 1H), 7.93 (s, 1H), 8.06 (ddd, J=8.66, 7.44, 6.02 Hz, 1H), 10.40 (s, 1H), 12.89 (br. s., 1H). HRMS (ESI) calcd for C21H20F2N5O [M+H]⁺ 396.1631, found 396.1650.

### N-[5-(3-Cyano-4-hydroxyphenyl)-1H-indazol-3-yl]-1-methylpiperidine-4-carboxamide hydrochloride (cpd 135) [(I), R1=3-cyano-4-hydroxyphenyl, R2=1-methylpiperidine, R3=H]

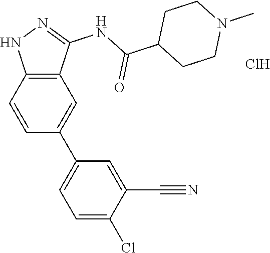 [SMILES: CN1CCC(C(=O)Nc2n[nH]c3ccc(-c4ccc(Cl)c(C#N)c4)cc23)CC1 ; Cl]

Obtained 0.032 g (98% of yield). 1H NMR (500 MHz, DMSO-d₆) δ ppm 1.88-2.03 (m, 2H), 2.11-2.19 (m, 2H), 2.76 (m, 1H), 2.81 (d, J=4.58 Hz, 3H), 2.97-3.09 (m, 2H), 3.53 (d, J=11.59 Hz, 2H), 7.17 (d, J=8.69 Hz, 1H), 7.54 (d, J=8.69 Hz, 1H), 7.65 (d, J=8.69 Hz, 1H), 7.79 (dd, J=8.69, 2.29 Hz, 1H), 7.86 (d, J=2.13 Hz, 1H), 7.94-8.00 (m, 1H), 9.69 (br. s., 1H), 10.59 (s, 1H), 11.24 (br. s., 1H), 12.81 (br. s., 1H). HRMS (ESI) calcd for C21H22N5O2 [M+H]⁺ 376.1768, found 376.1776.

### N-[5-(2-Cyano-5-fluorophenyl)-1H-indazol-3-yl]-1-methylpiperidine-4-carboxamide hydrochloride (cpd 136) [(I), R1=2-cyano-5-fluorophenyl, R2=1-methylpiperidine, R3=H]

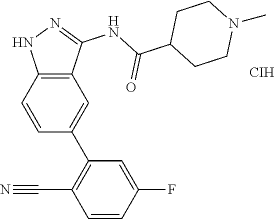

Obtained 0.052 g (87% of yield). ¹H NMR (500 MHz, DMSO-d₆) δ ppm 1.83-2.04 (m, 2H), 2.05-2.21 (m, 2H), 2.73 (m, 1H), 2.77 (d, J=4.58 Hz, 3H), 2.92-3.04 (m, 2H), 3.49 (d, J=11.59 Hz, 2H), 7.46 (td, J=8.46, 2.59 Hz, 1H), 7.53 (dd, J=9.68, 2.52 Hz, 1H), 7.55-7.63 (m, 2H), 8.01 (s, 1H), 8.06 (dd, J=8.69, 5.64 Hz, 1H), 9.62 (br. s., 1H), 10.68 (s, 1H), 12.97 (br. s., 1H). HRMS (ESI) calcd for C21H21FN5O [M+H]⁺ 378.1725, found 378.1730.

### N-[5-(2-Chloro-5-nitrophenyl)-1H-indazol-3-yl]-1-methylpiperidine-4-carboxamide trifluoroacetate (cpd 137) [(I), R1=2-chloro-5-nitrophenyl, R2=1-methylpiperidine, R3=H]

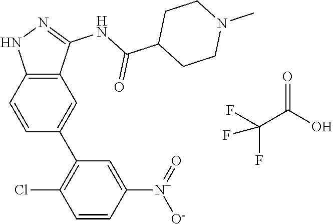 [SMILES: CN1CCC(C(=O)Nc2n[nH]c3ccc(-c4cc([N+](=O)[O-])ccc4Cl)cc23)CC1 ; O=C(O)C(F)(F)F]

Obtained 0.023 g (72% of yield). ¹H NMR (500 MHz, DMSO-d₆) δ ppm 1.76-1.92 (m, 2H), 2.04-2.15 (m, 2H), 2.71 (m, 1H), 2.79 (d, J=4.58 Hz, 3H), 2.93-3.06 (m, 2H), 3.49 (d, J=12.35 Hz, 3H), 7.47 (dd, J=8.69, 1.37 Hz, 1H), 7.58 (d, J=8.54 Hz, 1H), 7.86-7.97 (m, 2H), 8.18 (m, 1H), 8.25 (dd, J=8.77, 2.82 Hz, 1H), 9.19 (br. s., 1H), 10.65 (s, 1H), 12.93 (br. s., 1H). HRMS (ESI) calcd for C20H21ClN5O3 [M+H]⁺ 414.1328, found 414.1323.

### N-[5-(2,6-Difluoro-4-hydroxyphenyl)-1H-indazol-3-yl]-1-methylpiperidine-4-carboxamide trifluoroacetate (cpd 138) [(I), R1=2,6-difluoro-4-hydroxyphenyl, R2=1-methylpiperidine, R3=H]

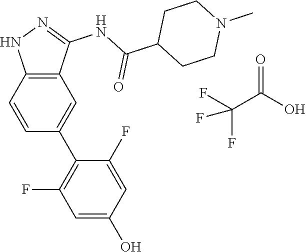

To a solution of N-[5-(2,6-difluoro-4-methoxyphenyl)-1-trityl-1H-indazol-3-yl]-1-methylpiperidine-4-carboxamide (0.076 g, 0.118 mmol) in dry DCM (2 mL), cooled at −10° C., boron tribromide (1.0 M DCM solution, (0.47 mL, 0.47 mmol) was added dropwise. The resulting suspension was stirred at room temperature overnight, then carefully basified with 33% aqueous ammonia (10 mL). The mixture was extracted with DCM (5×10 mL), the combined organic phase was dried with anhydrous Na₂SO₄, filtered and evaporated to dryness. The crude was purified by preparative HPLC to obtain N-[5-(2,6-difluoro-4-hydroxyphenyl)-1H-indazol-3-yl]-1-methylpiperidine-4-carboxamide as trifluoroacetate salt (0.045 g, 76% of yield). 1H NMR (500 MHz, DMSO-d₆) δ ppm 1.79-1.94 (m, 2H), 2.08-2.18 (m, 2H), 2.74 (m, 1H), 2.82 (d, J=4.42 Hz, 3H), 3.02 (m, 2H), 3.53 (d, J=12.20 Hz, 2H), 6.60 (d, J=9.61 Hz, 2H), 7.34 (d, J=8.54 Hz, 1H), 7.53 (d, J=8.69 Hz, 1H), 7.79 (s, 1H), 8.97-9.49 (m, 1H), 10.50 (br. s., 1H), 10.61 (s, 1H), 12.85 (br. s., 1H). HRMS (ESI) calcd for C20H21F2N4O2 [M+H]⁺ 387.1627, found 387.1631.

According to this same methodology, but employing suitable intermediate, the following compound was prepared:

### 1-Methyl-N-[5-(2,4,6-trifluoro-3-hydroxyphenyl)-1H-indazol-3-yl]piperidine-4-carboxamide (cpd 139) [(I), R1=2,4,6-trifluoro-3-hydroxyphenyl, R2=1-methylpiperidine, R3=H]

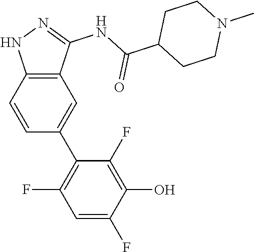 [SMILES: CN1CCC(C(=O)Nc2n[nH]c3ccc(-c4c(F)cc(F)c(O)c4F)cc23)CC1]

Starting from 1-methyl-N-[5-(2,4,6-trifluoro-3-methoxyphenyl)-1-trityl-1H-indazol-3-yl]piperidine-4-carboxamide. Flash column chromatography (DCM/MeOH/33% aqueous ammonia 90:10:0.1). Obtained 0.035 g (64% of yield). ¹H NMR (500 MHz, DMSO-d₆) δ ppm 1.57-1.71 (m, 2H), 1.78 (d, J=11.13 Hz, 2H), 1.86 (t, J=10.90 Hz, 2H), 2.15 (s, 3H), 2.39 (m, 1H), 2.80 (d, J=11.13 Hz, 2H), 7.24 (t, J=9.76 Hz, 1H), 7.33 (d, J=8.85 Hz, 1H), 7.52 (d, J=8.54 Hz, 1H), 7.82 (s, 1H), 10.36 (s, 1H), 12.80 (s, 1H). HRMS (ESI) calcd for C20H20F3N4O2 [M+H]⁺ 405.1533, found 405.1532.

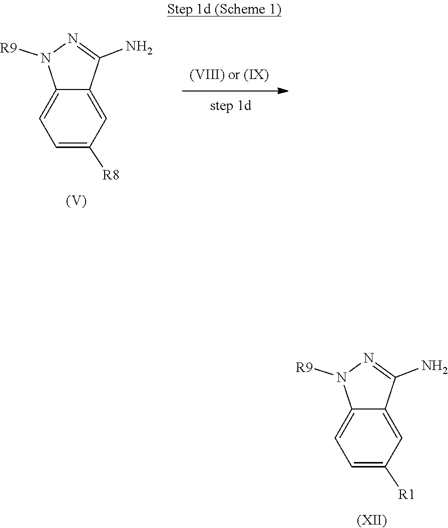

The compounds were prepared according to the same procedure reported for step 1b (Scheme 1).

### 5-(2,6-Dichloro-phenyl)-1-trityl-1H-indazol-3-ylamine [(XII), R1=2,6-Dichloro-phenyl, R9=methanetriyltribenzene]

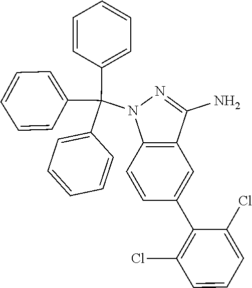 [SMILES: Nc1nn(C(c2ccccc2)(c2ccccc2)c2ccccc2)c2ccc(-c3c(Cl)cccc3Cl)cc12]

Starting from 5-bromo-1-trityl-1H-indazol-3-ylamine and 2-(2,6-dichlorophenyl)-4,4,5,5-tetramethyl-1,3,2-dioxaborolane. Flash column chromatography (Hexane/EtOAc 70:30). Obtained 3.13 g (91% of yield). ¹H NMR (500 MHz, DMSO-d₆) δ ppm 5.71 (s, 2H), 6.36 (d, J=8.69 Hz, 1H), 6.86 (dd, J=8.77, 1.75 Hz, 1H), 7.19-7.25 (m, 3H), 7.27-7.34 (m, 6H), 7.36-7.40 (m, 7H), 7.52-7.58 (m, 3H). HRMS (ESI) calcd for C32H24Cl2N3Na [M+H+Na]⁺542.1161, found 542.1164.

### 3-(3-Amino-1-trityl-1H-indazol-5-yl)-4-chloro-benzonitrile [(XII), R1=2-Chloro-5-cyanophenyl, R9=1,1′,1″-methanetriyltribenzene]

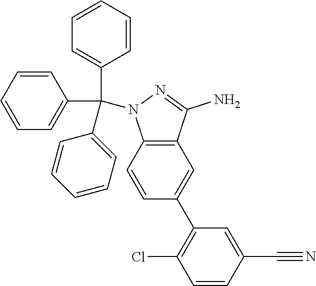 [SMILES: N#Cc1ccc(Cl)c(-c2ccc3c(c2)c(N)nn3C(c2ccccc2)(c2ccccc2)c2ccccc2)c1]

Starting from 5-bromo-1-trityl-1H-indazol-3-ylamine and (2-chloro-5-cyanophenyl)boronic acid. Flash column chromatography (Hexane/EtOAc 70:30). Obtained 0.750 g (85% of yield). 1H NMR (500 MHz, DMSO-d₆) δ ppm 5.75 (s, 2H), 6.36 (d, J=9.00 Hz, 1H), 7.12 (dd, J=8.77, 1.75 Hz, 1H), 7.20-7.25 (m, 3H), 7.28-7.33 (m, 7H), 7.35-7.39 (m, 6H), 7.75-7.78 (m, 1H), 7.82 (d, J=1.98 Hz, 2H), 7.88 (d, J=1.98 Hz, 1H). HRMS (ESI) calcd for C33H24ClN4Na [M+H+Na]⁺533.1503, found 533.1528.

### 5-(2-Chloro-6-fluorophenyl)-1-trityl-1H-indazol-3-amine [(XII), R1=2-chloro-6-fluorophenyl, R9=methanetriyltribenzene]

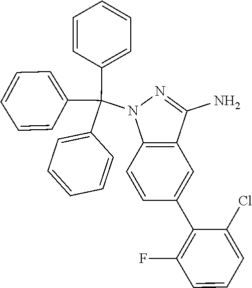 [SMILES: Nc1nn(C(c2ccccc2)(c2ccccc2)c2ccccc2)c2ccc(-c3c(F)cccc3Cl)cc12]

Starting from 5-bromo-1-trityl-1H-indazol-3-ylamine and (2-chloro-6-fluorophenyl)boronic acid. Flash column chromatography Hexane/EtOAc 80:20). Obtained 0.27 g (48% of yield). LCMS ESI) m/z 504 (M+H)⁺

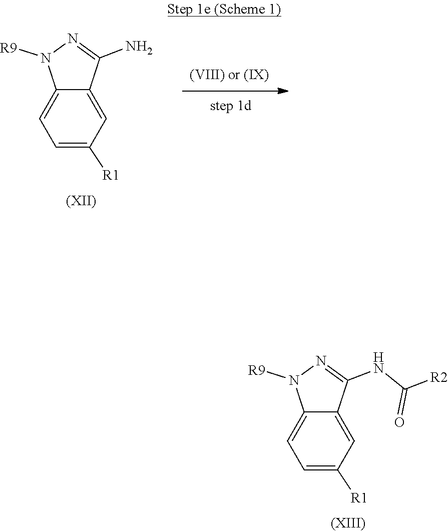

The compounds were prepared according to the same procedure reported for step 1a (Scheme 1).

### N-[5-(2,6-Dichlorophenyl)-1-trityl-1H-indazol-3-yl]-3-oxocyclobutanecarboxamide [(XIII), R1=2,6-dichlorophenyl, R2=3-oxocyclobutane, R9=1,1′,1″-methanetriyltribenzene]

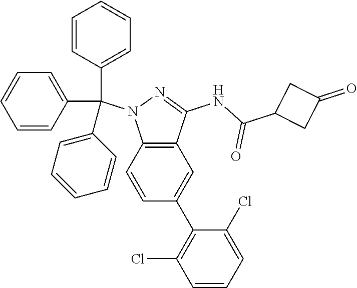 [SMILES: O=C1CC(C(=O)Nc2nn(C(c3ccccc3)(c3ccccc3)c3ccccc3)c3ccc(-c4c(Cl)cccc4Cl)cc23)C1]

Starting from 5-(2,6-dichlorophenyl)-1-trityl-1H-indazol-3-amine and 3-oxo-cyclobutanecarboxylic acid. Flash column chromatography (Hexane/EtOAc 70:30). Obtained 0.054 g (76% of yield). ¹H NMR (500 MHz, DMSO-d₆) δ ppm 3.26 (m, 4H), 3.45 (m, 1H), 6.48 (d, J=8.85 Hz, 1H), 6.94 (d, J=8.69 Hz, 1H), 7.23-7.38 (m, 15H), 7.40 (dd, J=8.46, 7.70 Hz, 1H), 7.56 (d, J=8.08 Hz, 2H), 7.77 (s, 1H), 10.87 (s, 1H). HRMS (ESI) calcd for C37H28Cl2N3O2Na [M+H+Na]⁺638.1372, found 638.1383.

### N-[5-(2-Chloro-5-cyanophenyl)-1-trityl-1H-indazol-3-yl]-3-oxocyclobutanecarboxamide [(XIII), R1=2-chloro-5-cyanophenyl, R2=3-oxocyclobutane, R9=1,1′,1″-methanetriyltribenzene]

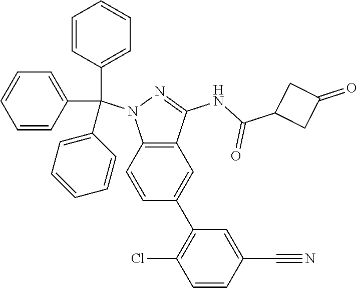 [SMILES: N#Cc1ccc(Cl)c(-c2ccc3c(c2)c(NC(=O)C2CC(=O)C2)nn3C(c2ccccc2)(c2ccccc2)c2ccccc2)c1]

Starting from 3-(3-amino-1-trityl-1H-indazol-5-yl)-4-chlorobenzonitrile and 3-oxo-cyclobutanecarboxylic acid. Flash column chromatography (Hexane/EtOAc 70:30). Obtained 0.580 g (89% of yield). ¹H NMR (500 MHz, DMSO-d₆) δ ppm 3.24-3.32 (m, 2H), 3.45 (m, 1H), 6.48 (d, J=8.85 Hz, 1H), 7.16 (d, J=8.85 Hz, 1H), 7.24 (d, J=7.47 Hz, 6H), 7.28-7.31 (m, 3H), 7.34-7.37 (m, 6H), 7.77 (d, J=8.39 Hz, 1H), 7.84 (dd, J=8.24, 1.98 Hz, 1H), 7.88 (s, 1H), 7.93 (s, 1H), 10.89 (s, 1H). HRMS (ESI) calcd for C38H28ClN4O2Na [M+H+Na]⁺629.1715, found 629.1725.

### N-[5-(2,6-Dichlorophenyl)-1-trityl-1H-indazol-3-yl]-4-oxocyclohexanecarboxamide [(XIII), R1=2,6-dichlorophenyl, R2=4-oxocyclohexane, R9=1,1′,1″-methanetriyltribenzene]

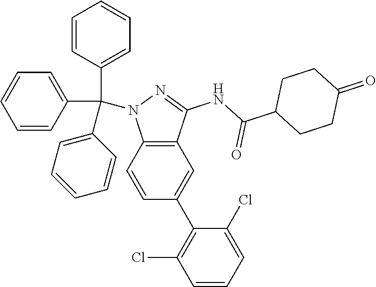 [SMILES: O=C1CCC(C(=O)Nc2nn(C(c3ccccc3)(c3ccccc3)c3ccccc3)c3ccc(-c4c(Cl)cccc4Cl)cc23)CC1]

Starting from 5-(2,6-dichloro-phenyl)-1-trityl-1H-indazol-3-ylamine and 4-oxo-cyclohexanecarboxylic acid. Flash column chromatography (Hexane/EtOAc 50:50). Obtained 0.121 g (49% of yield). ¹H NMR (500 MHz, DMSO-d₆) δ ppm 1.73-1.92 (m, 2H), 2.12 (d, J=3.36 Hz, 2H), 2.27 (d, J=14.18 Hz, 2H), 2.37-2.44 (m, 2H), 2.93 (m, 1H), 6.47 (d, J=8.85 Hz, 1H), 6.94 (d, J=8.54 Hz, 1H), 7.28 (dd, J=18.15, 7.47 Hz, 9H), 7.32-7.38 (m, 6H), 7.40 (dd, J=8.39, 7.78 Hz, 1H), 7.55 (d, J=8.08 Hz, 2H), 7.68 (s, 1H), 10.63 (s, 1H). HRMS (ESI) calcd for C39H32Cl2N3O2Na [M+H+Na]⁺666.1685, found 666.1703.

### N-[5-(2,6-Dichlorophenyl)-1-trityl-1H-indazol-3-yl]tetrahydro-2H-pyran-4-carboxamide [(XIII), R1=2,6-dichlorophenyl, R2=tetrahydro-2H-pyran, R9=1,1′,1″-methanetriyltribenzene]

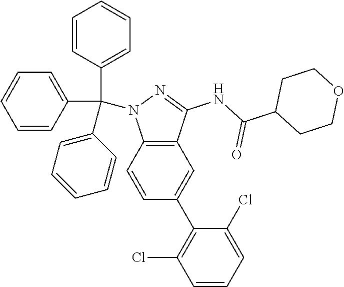 [SMILES: O=C(Nc1nn(C(c2ccccc2)(c2ccccc2)c2ccccc2)c2ccc(-c3c(Cl)cccc3Cl)cc12)C1CCOCC1]

Starting from 5-(2,6-dichlorophenyl)-1-trityl-1H-indazol-3-ylamine and tetrahydro-2H-pyran-4-carboxylic acid. Flash column chromatography (Hexane/EtOAc 50:50). Obtained 0.058 g (68% of yield). ¹H NMR (500 MHz, DMSO-d₆) δ ppm 1.56-1.67 (m, 2H), 1.67-1.78 (m, 2H), 2.71 (tt, J=10.98, 4.10 Hz, 1H), 3.30 (t, J=12.10 Hz, 2H), 3.87 (d, J=9.91 Hz, 2H), 6.47 (d, J=8.85 Hz, 1H), 6.94 (d, J=8.54 Hz, 1H), 7.21-7.28 (m, 6H), 7.28-7.32 (m, 3H), 7.32-7.38 (m, 6H), 7.41 (t, J=8.54 Hz, 1H), 7.56 (d, J=8.08 Hz, 2H), 7.67 (s, 1H), 10.51 (s, 1H). HRMS (ESI) calcd for C38H32Cl2N3O2 [M+H]⁺ 632.1866, found 632.1854.

### tert-Butyl 3-{[5-(2,6-dichlorophenyl)-1-trityl-1H-indazol-3-yl]carbamoyl}piperidine-1-carboxylate [(XIII), R1=2,6-dichlorophenyl, R2=tert-butyl piperidine-1-carboxylate, R9=1,1′,1″-methanetriyltribenzene]

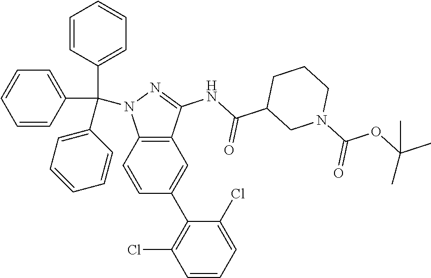 [SMILES: CC(C)(C)OC(=O)N1CCCC(C(=O)Nc2nn(C(c3ccccc3)(c3ccccc3)c3ccccc3)c3ccc(-c4c(Cl)cccc4Cl)cc23)C1]

Starting from 5-(2,6-dichlorophenyl)-1-trityl-1H-indazol-3-ylamine and 1-(tert-butoxycarbonyl)piperidine-3-carboxylic acid. Flash column chromatography (Hexane/EtOAc 75:25). Obtained 0.110 g (95% of yield). LCMS (ESI) m/z 731 (M+H)⁺.

### 1-Cyclopropyl-N-[5-(2,6-dichlorophenyl)-1-trityl-1H-indazol-3-yl]piperidine-4-carboxamide [(XIII), R1=2,6-dichlorophenyl, R2=1-cyclopropylpiperidine, R9=1,1′,1″-methanetriyltribenzene]

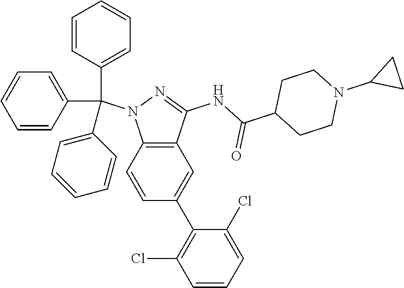 [SMILES: O=C(Nc1nn(C(c2ccccc2)(c2ccccc2)c2ccccc2)c2ccc(-c3c(Cl)cccc3Cl)cc12)C1CCN(C2CC2)CC1]

Starting from 5-(2,6-dichlorophenyl)-1-triyl-1H-indazol-3-ylamine and 1-cyclopropylpiperidine-4-carboxylic acid. Flash column chromatography (DCM/MeOH 95:5). Obtained 0.066 g (79% of yield). LCMS (ESI) m/z 671 (M+H)⁺.

### N-[5-(2,6-Dichlorophenyl)-1-trityl-1H-indazol-3-yl]-1-phenylpiperidine-4-carboxamide [(XIII), R1=2,6-dichlorophenyl, R2=1-phenylpiperidine, R9=1,1′,1″-methanetriyltribenzene]

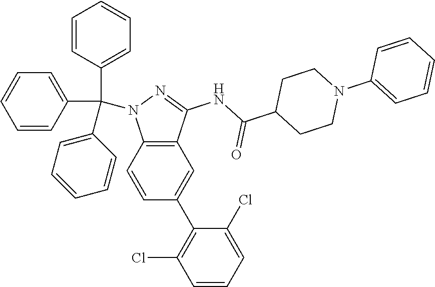 [SMILES: O=C(Nc1nn(C(c2ccccc2)(c2ccccc2)c2ccccc2)c2ccc(-c3c(Cl)cccc3Cl)cc12)C1CCN(c2ccccc2)CC1]

Starting from 5-(2,6-dichlorophenyl)-1-trityl-1H-indazol-3-ylamine and 1-phenylpiperidine-4-carboxylic acid. Flash column chromatography (DCM/MeOH 97:3). Obtained 0.041 g (90% of yield). LCMS (ESI) m/z 707 (M+H)⁺.

### N-[5-(2,6-Dichlorophenyl)-1-trityl-1H-indazol-3-yl]-1-azabicyclo[2.2.2]octane-4-carboxamide [(XIII), R1=2,6-dichlorophenyl, R2=1-azabicyclo[2.2.2]octane, R9=1,1′,1″-methanetriyltribenzene]

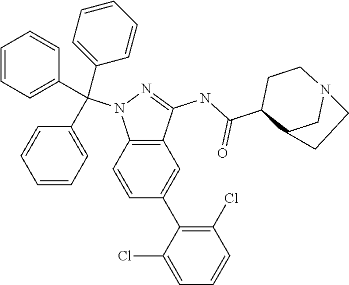 [SMILES: O=C(Nc1nn(C(c2ccccc2)(c2ccccc2)c2ccccc2)c2ccc(-c3c(Cl)cccc3Cl)cc12)[C@H]1CCN2CCC1C2]

Starting from 5-(2,6-dichlorophenyl)-1-trityl-1H-indazol-3-ylamine and 1-azabicyclo[2.2.2]octane-4-carboxylic acid. Flash column chromatography (DCM/MeOH/33% aqueous ammonia 95:5:0.2). Obtained 0.120 g (82% of yield). LCMS (ESI) m/z 657 (M+H)⁺.

### N-[5-(2-Chloro-6-fluorophenyl)-1-trityl-1H-indazol-3-yl]-1-methylpiperidine-3-carboxamide [(XIII), R1=2-chloro-6-fluorophenyl, R2=1-methylpiperidine, R9=1,1′,1″-methanetriyltribenzene]

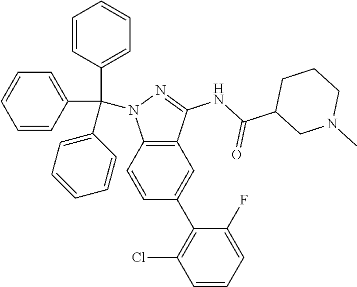 [SMILES: CN1CCCC(C(=O)Nc2nn(C(c3ccccc3)(c3ccccc3)c3ccccc3)c3ccc(-c4c(F)cccc4Cl)cc23)C1]

Starting from 5-(2-chloro-6-fluorophenyl)-1-trityl-1H-indazol-3-ylamine and 1-methylpiperidine-3-carboxylic acid. Flash column chromatography (DCM/MeOH/33% aqueous ammonia 95:5:0.5). Obtained 0.140 g (72% of yield). ¹H NMR (500 MHz, DMSO-d₆) δ ppm 1.32-1.56 (m, 2H), 1.65 (m, 1H), 1.81-1.88 (m, 2H), 2.03 (m, 1H), 2.17 (s, 3H), 2.68-2.76 (m, 2H), 2.87 (d, J=10.22 Hz, 1H), 6.50 (d, J=9.00 Hz, 1H), 7.06 (d, J=9.00 Hz, 1H), 7.24-7.42 (m, 16H), 7.43-7.51 (m, 2H), 7.74 (s, 1H), 10.58 (s, 1H). HRMS (ESI) calcd for C39H35ClFN4O [M+H]⁺ 629.2478, found 629.2484.

### tert-Butyl (2S)-2-(2-{[5-(2,6-dichlorophenyl)-1-trityl-1H-indazol-3-yl]amino}-2-oxoethyl)pyrrolidine-1-carboxylate [(XIII), R1=2,6-dichlorophenyl, R2=tert-butyl (2R)-2-methylpyrrolidine-1-carboxylate, R9=1,1′,1″-methanetriyltribenzene]

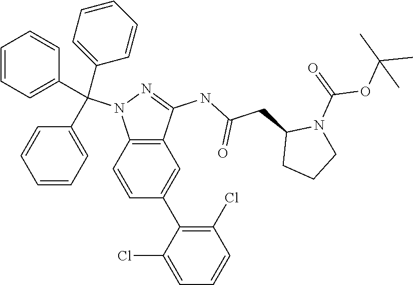 [SMILES: CC(C)(C)OC(=O)N1CCC[C@H]1CC(=O)Nc1nn(C(c2ccccc2)(c2ccccc2)c2ccccc2)c2ccc(-c3c(Cl)cccc3Cl)cc12]

Starting from 5-(2,6-dichlorophenyl)-1-trityl-1H-indazol-3-ylamine and [(2S)-1-(tert-butoxycarbonyl)pyrrolidin-2-yl]acetic acid. Flash column chromatography (Hexane/EtOAc 70:30). Obtained 0.051 g (88% of yield). LCMS (ESI) m/z 731 (M+H)⁺.

### tert-Butyl 4-(2-{[5-(2,6-dichlorophenyl)-1-trityl-1H-indazol-3-yl]amino}-2-oxoethyl)piperidine-1-carboxylate [(XIII), R1=2,6-dichlorophenyl, R2=tert-butyl 4-methylpiperidine-1-carboxylate, R9=methanetriyltribenzene]

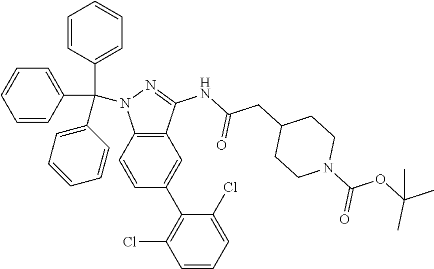 [SMILES: CC(C)(C)OC(=O)N1CCC(CC(=O)Nc2nn(C(c3ccccc3)(c3ccccc3)c3ccccc3)c3ccc(-c4c(Cl)cccc4Cl)cc23)CC1]

Starting from 5-(2,6-dichlorophenyl)-1-trityl-1H-indazol-3-ylamine and [1-(tert-butoxycarbonyl)piperidin-4-yl]acetic acid. Flash column chromatography (Hexane/EtOAc 50:50). Obtained 0.068 g (89% of yield). ¹H NMR (500 MHz, DMSO-d₆) δ ppm 1.05-1.14 (m, 2H), 1.37 (s., 9H), 1.61-1.66 (m, 2H), 2.28 (m, 1H), 3.46-3.52 (m, 2H), 3.86-3.90 (m, 2H), 6.46 (d, J=9.00 Hz, 1H), 6.94 (dd, J=9.15, 1.68 Hz, 1H), 7.25-7.31 (m, 9H), 7.33-7.36 (m, 6H), 7.41 (dd, J=8.54, 7.93 Hz, 1H), 7.55 (d, J=8.08 Hz, 2H), 7.69 (br s, 1H), 10.53 (br. s., 1H). HRMS (ESI) calcd for C44H43Cl2N4O3Na [M+H+Na]⁺767.2526, found 767.2520.

### tert-Butyl [(trans-4-{[5-(2,6-dichlorophenyl)-1-trityl-1H-indazol-3-yl]carbamoyl)cyclohexyl)methyl]carbamate [(XIII), R1=2,6-dichlorophenyl, R2=tert-butyl(cyclohexylmethyl)carbamate, R9=1,1′,1″-methanetriyltribenzene]

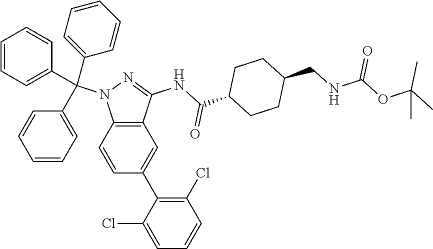 [SMILES: CC(C)(C)OC(=O)NC[C@H]1CC[C@H](C(=O)Nc2nn(C(c3ccccc3)(c3ccccc3)c3ccccc3)c3ccc(-c4c(Cl)cccc4Cl)cc23)CC1]

Starting from 5-(2,6-dichlorophenyl)-1-trityl-1H-indazol-3-ylamine and trans-4-{[(tert-butoxycarbonyl)amino]-methyl}cyclohexanecarboxylic acid. Flash column chromatography (Hexane/EtOAc 60:40). Obtained 0.093 g (80% of yield). LCMS (ESI) m/z 759 (M+H)⁺.

### tert-Butyl 3-{[5-(2-chloro-6-fluorophenyl)-1-trityl-1H-indazol-3-yl]carbamoyl}piperidine-1-carboxylate [(XIII), R1=2-chloro-6-fluorophenyl, R2=tert-butyl piperidine-1-carboxylate, R9=methanetriyltribenzene]

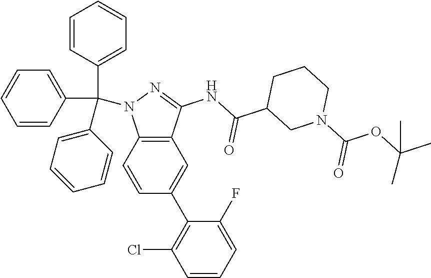 [SMILES: CC(C)(C)OC(=O)N1CCCC(C(=O)Nc2nn(C(c3ccccc3)(c3ccccc3)c3ccccc3)c3ccc(-c4c(F)cccc4Cl)cc23)C1]

Starting from 5-(2-chloro-6-fluorophenyl)-1-trityl-1H-indazol-3-ylamine and 1-(tert-butoxycarbonyl)piperidine-3-carboxylic acid. Flash column chromatography (Hexane/EtOAc 70:30). Obtained 0.097 g (96% of yield). ¹H NMR (401 MHz, DMSO-d₆) δ ppm 1.36 (s, 9H), 1.52-1.78 (m, 2H), 1.96 (d, J=11.74 Hz, 2H), 2.59 (m, 1H), 2.69-3.21 (m, 2H), 3.80 (d, J=12.81 Hz, 1H), 4.05 (q, J=7.12 Hz, 1H), 6.44 (m, 1H), 6.92-6.98 (m, 3H), 7.26-7.33 (m, 9H), 7.36-7.45 (m, 7H), 7.47-7.56 (m, 3H), 7.90 (d, J=9.03 Hz, 2H), 10.47 (s, 1H).

### tert-Butyl [3-(3-{[5-(2-chloro-6-fluorophenyl)-1-trityl-1H-indazol-3-yl]carbamoyl}piperidin-1-yl)propyl]-carbamate [(XIII), R1=2-chloro-6-fluorophenyl, R2=tert-butyl [3-(piperidin-1-yl)propyl]carbamate, R9=1,1′,1″-methanetriyltribenzene]

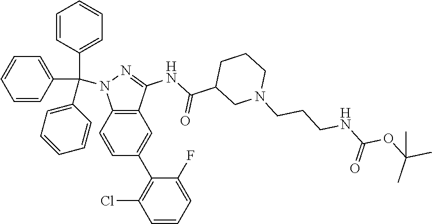 [SMILES: CC(C)(C)OC(=O)NCCCN1CCCC(C(=O)Nc2nn(C(c3ccccc3)(c3ccccc3)c3ccccc3)c3ccc(-c4c(F)cccc4Cl)cc23)C1]

Starting from 5-(2-chloro-6-fluorophenyl)-1-trityl-1H-indazol-3-ylamine and 1-{3-[(tert-butoxycarbonyl)amino]propyl}-piperidine-3-carboxylic acid. Flash column chromatography (DCM/MeOH 95:5). Obtained 0.093 g (93% of yield). ¹H NMR (500 MHz, DMSO-d₆) δ ppm 1.32-1.38 (m, 9H), 1.47-1.54 (m, 3H), 1.62-1.68 (m, 2H), 1.80-1.88 (m, 2H), 2.04 (m, 1H), 2.24 (m, 2H), 2.66-2.72 (m, 3H), 2.90 (m, 2H), 6.47 (d, J=9.00 Hz, 1H), 6.75 (t, J=6.41 Hz, 1H), 7.02 (d, J=9.00 Hz, 1H), 7.22-7.37 (m, 16H), 7.40-7.47 (m, 2H), 7.72 (s, 1H), 10.57 (s, 1H). HRMS (ESI) calcd for C46H48ClFN5O3 [M+H]⁺ 772.3424, found 772.3424.

### 4-(Benzyloxy)-N-[5-(2,6-dichlorophenyl)-1-trityl-1H-indazol-3-yl]cyclohexanecarboxamide [(XIII), R1=2,6-dichlorophenyl, R2=[(cyclohexyloxy)methyl]benzene, R9=1,1′,1″-methanetriyltribenzene]

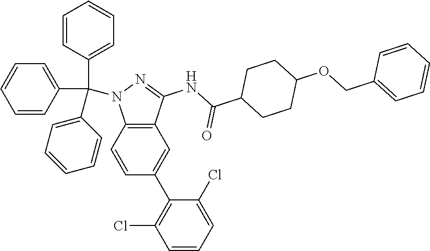 [SMILES: O=C(Nc1nn(C(c2ccccc2)(c2ccccc2)c2ccccc2)c2ccc(-c3c(Cl)cccc3Cl)cc12)C1CCC(OCc2ccccc2)CC1]

Starting from 5-(2,6-dichlorophenyl)-1-trityl-1H-indazol-3-ylamine and 4-(benzyloxy)cyclohexanecarboxylic acid. Flash column chromatography (Hexane/EtOAc 80:20). Obtained 0.024 g (22% of yield). ¹H NMR (500 MHz, DMSO-d₆) δ ppm 1.12-1.23 (m, 2H), 1.41-1.46 (m, 2H), 1.56-1.59 (m, 1H), 1.71-1.82 (m, 1H), 1.85-1.95 (m, 2H), 3.32 (m, 1H), 3.60 (m, 1H), 4.32-4.59 (m, 2H), 6.46 (d, J=9.15 Hz, 1H), 6.93 (d, J=9.00 Hz, 1H), 7.22-7.37 (m, 20H), 7.40 (m, 1H), 7.54 (d, J=8.08 Hz, 2H), 7.65 (s, 1H), 10.44 (d, J=19.98 Hz, 1H). HRMS (ESI) calcd for C46H40Cl2N3O2 [M+H]⁺ 736.2492, found 736.2489.

### tert-Butyl (3R)-3-{[5-(2,6-dichlorophenyl)-1-trityl-1H-indazol-3-yl]carbamoyl}pyrrolidine-1-carboxylate [(XIII), R1=2,6-dichlorophenyl, R2=tert-butyl pyrrolidine-1-carboxylate, R9=

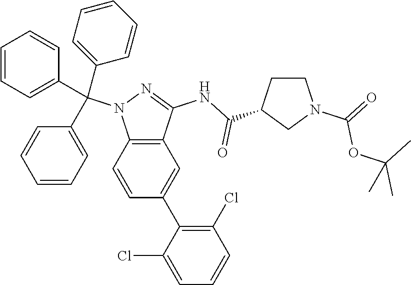 [SMILES: CC(C)(C)OC(=O)N1CC[C@@H](C(=O)Nc2nn(C(c3ccccc3)(c3ccccc3)c3ccccc3)c3ccc(-c4c(Cl)cccc4Cl)cc23)C1]

Starting from 5-(2,6-dichlorophenyl)-1-trityl-1H-indazol-3-ylamine and (3R)-1-(tert-butoxycarbonyl)pyrrolidine-3-carboxylic acid. Flash column chromatography (Hexane/EtOAc 70:30). Obtained 0.090 g (89% of yield). ¹H NMR (500 MHz, DMSO-d₆) δ ppm 1.33-1.41 (m, 9H), 1.95-2.13 (m, 2H), 3.14-3.27 (m, 2H), 3.36-3.42 (m, 1H), 3.39 (m, 1H), 3.49 (m, 1H), 6.47 (d, J=9.00 Hz, 1H), 6.94 (d, J=9.00 Hz, 1H), 7.21-7.27 (m, 6H), 7.28-7.31 (m, 3H), 7.32-7.37 (m, 6H), 7.40 (t, J=8.24 Hz, 1H), 7.55 (d, J=8.24 Hz, 2H), 7.66 (s, 1H), 10.69 (s, 1H). HRMS (ESI) calcd for C42H39Cl2N4O3Na [M+H+Na]⁺739.2213, found 739.2206.

### tert-Butyl (3S)-3-{[5-(2,6-dichlorophenyl)-1-trityl-1H-indazol-3-yl]carbamoyl}pyrrolidine-1-carboxylate [(XIII), R1=2,6-dichlorophenyl, R2=tert-butyl pyrrolidine-1-carboxylate, R9=methanetriyltribenzene]

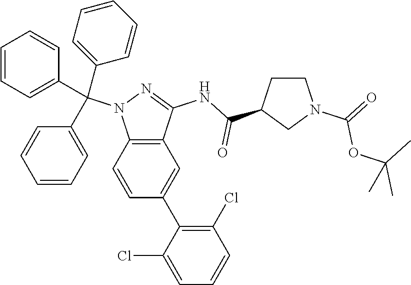 [SMILES: CC(C)(C)OC(=O)N1CC[C@H](C(=O)Nc2nn(C(c3ccccc3)(c3ccccc3)c3ccccc3)c3ccc(-c4c(Cl)cccc4Cl)cc23)C1]

Starting from 5-(2,6-dichlorophenyl)-1-trityl-1H-indazol-3-ylamine and (3S)-1-(tert-butoxycarbonyl)pyrrolidine-3-carboxylic acid. Flash column chromatography (Hexane/EtOAc 70:30). Obtained 0.095 g (91% of yield). ¹H NMR (500 MHz, DMSO-d₆) δ ppm 1.33-1.41 (m, 9H), 1.95-2.13 (m, 2H), 3.14-3.27 (m, 2H), 3.36-3.42 (m, 1H), 3.39 (m, 1H), 3.49 (m, 1H), 6.47 (d, J=9.00 Hz, 1H), 6.94 (d, J=9.00 Hz, 1H), 7.21-7.27 (m, 6H), 7.28-7.31 (m, 3H), 7.32-7.37 (m, 6H), 7.40 (t, J=8.24 Hz, 1H), 7.55 (d, J=8.24 Hz, 2H), 7.66 (s, 1H), 10.69 (s, 1H). HRMS (ESI) calcd for C42H39Cl2N4O3Na [M+H+Na]⁺739.2213, found 739.2208.

### tert-Butyl (3R)-3-{[5-(2,6-dichlorophenyl)-1-trityl-1H-indazol-3-yl]carbamoyl}piperidine-1-carboxylate [(XIII), R1=2,6-dichlorophenyl, R2=tert-butyl piperidine-1-carboxylate, R9=1,1′,1″-methanetriyltribenzene]

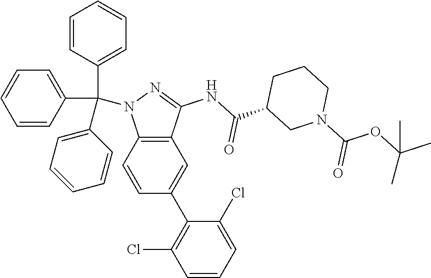 [SMILES: CC(C)(C)OC(=O)N1CCC[C@@H](C(=O)Nc2nn(C(c3ccccc3)(c3ccccc3)c3ccccc3)c3ccc(-c4c(Cl)cccc4Cl)cc23)C1]

Starting from 5-(2,6-dichlorophenyl)-1-trityl-1H-indazol-3-ylamine and (3R)-1-(tert-butoxycarbonyl)piperidine-3-carboxylic acid. Flash column chromatography (Hexane/EtOAc 70:30). Obtained 0.140 g (93% of yield). ¹H NMR (401 MHz, DMSO-d₆) δ ppm ppm 1.37 (s, 9H), 1.54-1.77 (m, 2H), 1.94 (d, J=11.74 Hz, 2H), 2.57 (m, 1H), 2.68-3.20 (m, 2H), 3.81 (d, J=12.81 Hz, 1H), 4.03 (q, J=7.12 Hz, 1H), 6.44 (dd, J=9.03, 0.85 Hz, 1H), 6.91-6.97 (m, 3H), 7.25-7.31 (m, 9H), 7.34-7.43 (m, 7H), 7.49-7.55 (m, 3H), 7.91 (d, J=9.03 Hz, 2H), 10.50 (s, 1H). HRMS (ESI) calcd for C43H41Cl2N4O3Na [M+H+Na]⁺753.7087, found 753.7082.

### tert-Butyl (3S)-3-{[5-(2,6-dichlorophenyl)-1-trityl-1H-indazol-3-yl]carbamoyl}piperidine-1-carboxylate [(XIII), R1=2,6-dichlorophenyl, R2=tert-butyl piperidine-1-carboxylate, R9=1,1′,1″-methanetriyltribenzene]

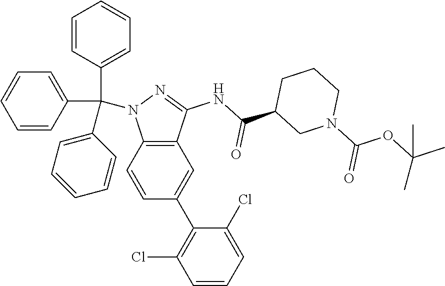 [SMILES: CC(C)(C)OC(=O)N1CCC[C@H](C(=O)Nc2nn(C(c3ccccc3)(c3ccccc3)c3ccccc3)c3ccc(-c4c(Cl)cccc4Cl)cc23)C1]

Starting from 5-(2,6-dichlorophenyl)-1-trityl-1H-indazol-3-ylamine and (3S)-1-(tert-butoxycarbonyl)piperidine-3-carboxylic acid. Flash column chromatography (Hexane/EtOAc 70:30). Obtained 0.075 g (91% of yield). ¹H NMR (401 MHz, DMSO-d₆) δ ppm ppm 1.37 (s, 9H), 1.54-1.77 (m, 2H), 1.94 (d, J=11.74 Hz, 2H), 2.57 (m, 1H), 2.68-3.20 (m, 2H), 3.81 (d, J=12.81 Hz, 1H), 4.03 (q, J=7.12 Hz, 1H), 6.44 (dd, J=9.03, 0.85 Hz, 1H), 6.91-6.97 (m, 3H), 7.25-7.31 (m, 9H), 7.34-7.43 (m, 7H), 7.49-7.55 (m, 3H), 7.91 (d, J=9.03 Hz, 2H), 10.50 (s, 1H). HRMS (ESI) calcd for C43H41Cl2N4O3Na [M+H+Na]⁺753.7087, found 753.7090.

### tert-Butyl 3-{[5-(2-chloro-5-cyanophenyl)-1-trityl-1H-indazol-3-yl]carbamoyl}piperidine-1-carboxylate [(XIII), R1=2-chloro-5-cyanophenyl, R2=tert-butyl piperidine-1-carboxylate, R9=methanetriyltribenzene]

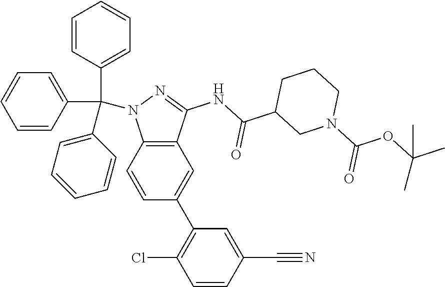 [SMILES: CC(C)(C)OC(=O)N1CCCC(C(=O)Nc2nn(C(c3ccccc3)(c3ccccc3)c3ccccc3)c3ccc(-c4cc(C#N)ccc4Cl)cc23)C1]

Starting from 3-(3-amino-1-trityl-1H-indazol-5-yl)-4-chlorobenzonitrile and 1-(tert-butoxycarbonyl)piperidine-3-carboxylic acid. Flash column chromatography (Hexane/EtOAc 80:20). Obtained 0.038 g (90% of yield). ¹H NMR (500 MHz, DMSO-d₆) δ ppm 1.36 (s, 9H), 1.57-1.74 (m, 2H), 1.90-1.96 (m, 1H), 2.58 (m., 1H), 2.75-2.83 (m, 1H), 3.77-3.80 (m, 1H), 6.46 (d, J=8.85 Hz, 1H), 7.16 (d, J=8.69 Hz, 1H), 7.23 (d, J=7.63 Hz, 6H), 7.28-7.31 (m, 3H), 7.33-7.36 (m, 6H), 7.77 (d, J=8.24 Hz, 1H), 7.82-7.91 (m, 3H), 10.65 (s, 1H). HRMS (ESI) calcd for C44H41ClN5O3Na [M+H+Na]⁺744.2712, found 744.2715.

### tert-Butyl (3R)-3-{[5-(2-chloro-6-fluorophenyl)-1-trityl-1H-indazol-3-yl]carbamoyl}piperidine-1-carboxylate [(XIII), R1=2-chloro-6-fluorophenyl, R2=tert-butyl piperidine-1-carboxylate, R9=methanetriyltribenzene]

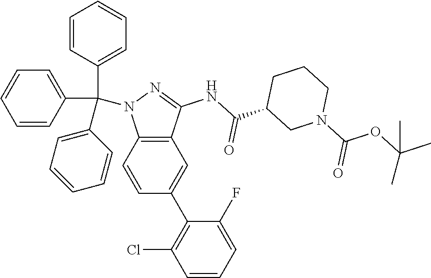 [SMILES: CC(C)(C)OC(=O)N1CCC[C@@H](C(=O)Nc2nn(C(c3ccccc3)(c3ccccc3)c3ccccc3)c3ccc(-c4c(F)cccc4Cl)cc23)C1]

Starting from 5-(2-chloro-6-fluorophenyl)-1-trityl-1H-indazol-3-ylamine and (3R)-1-(tert-butoxycarbonyl)piperidine-3-carboxylic acid. Flash column chromatography (Hexane/EtOAc 70:30). Obtained 0.120 g (89% of yield). LCMS (ESI) m/z 715 (M+H)⁺.

### tert-Butyl (3S)-3-{[5-(2-chloro-6-fluorophenyl)-1-trityl-1H-indazol-3-yl]carbamoyl}piperidine-1-carboxylate [(XIII), R1=2-chloro-6-fluorophenyl, R2=tert-butyl piperidine-1-carboxylate, R9=methanetriyltribenzene]

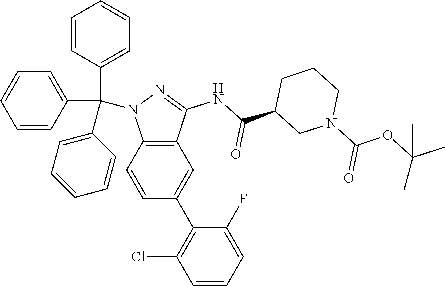 [SMILES: CC(C)(C)OC(=O)N1CCC[C@H](C(=O)Nc2nn(C(c3ccccc3)(c3ccccc3)c3ccccc3)c3ccc(-c4c(F)cccc4Cl)cc23)C1]

Starting from 5-(2-chloro-6-fluorophenyl)-1-trityl-1H-indazol-3-ylamine and (3S)-1-(tert-butoxycarbonyl)piperidine-3-carboxylic acid. Flash column chromatography (Hexane/EtOAc 70:30). Obtained 0.099 g (87% of yield). LCMS (ESI) m/z 715 (M+H)⁺.

### tert-Butyl (3R)-3-{[5-(2-chloro-5-cyanophenyl)-1-trityl-1H-indazol-3-yl]carbamoyl}piperidine-1-carboxylate [(XIII), R1=2-chloro-5-cyanophenyl, R2=tert-butyl piperidine-1-carboxylate, R9=methanetriyltribenzene]

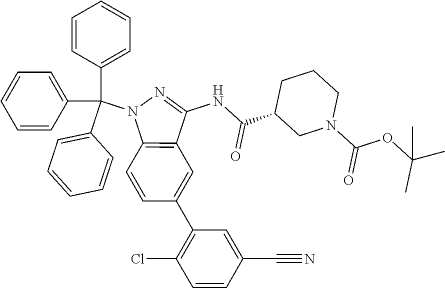 [SMILES: CC(C)(C)OC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from 3-(3-amino-1-trityl-1H-indazol-5-yl)-4-chlorobenzonitrile and (3R)-1-(tert-butoxycarbonyl)piperidine-3-carboxylic acid. Flash column chromatography (Hexane/EtOAc 70:30). Obtained 0.510 g (94% of yield). ¹H NMR (500 MHz, DMSO-d₆) δ ppm 1.36 (s, 9H), 1.57-1.74 (m, 2H), 1.90-1.96 (m, 1H), 2.58 (m., 1H), 2.75-2.83 (m, 1H), 3.77-3.80 (m, 1H), 6.46 (d, J=8.85 Hz, 1H), 7.16 (d, J=8.69 Hz, 1H), 7.23 (d, J=7.63 Hz, 6H), 7.28-7.31 (m, 3H), 7.33-7.36 (m, 6H), 7.77 (d, J=8.24 Hz, 1H), 7.82-7.91 (m, 3H), 10.65 (s, 1H). HRMS (ESI) calcd for C44H41ClN5O3Na [M+H+Na]⁺744.2712, found 744.2719.

### tert-Butyl (3-{[5-(2,6-dichlorophenyl)-1-trityl-1H-indazol-3-yl]amino}-3-oxopropyl)carbamate [(XIII), R1=2,6-dichlorophenyl, R2=tert-butyl ethylcarbamate, R9=1,1′,1″-methanetriyltribenzene]

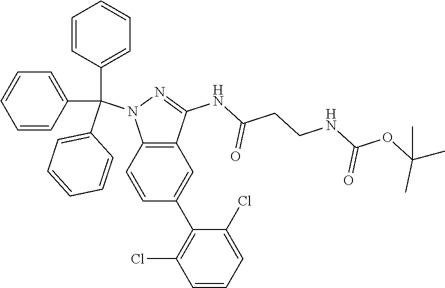 [SMILES: CC(C)(C)OC(=O)NCCC(=O)Nc1nn(C(c2ccccc2)(c2ccccc2)c2ccccc2)c2ccc(-c3c(Cl)cccc3Cl)cc12]

Starting from 5-(2,6-dichlorophenyl)-1-trityl-1H-indazol-3-ylamine and N-(tert-butoxycarbonyl)-β-alanine. Flash column chromatography (Hexane/EtOAc 70:30). Obtained 0.042 g (88% of yield). LCMS (ESI) m/z 691 (M+H)⁺.

### tert-Butyl (3R)-3-{[5-(2-chloro-5-cyanophenyl)-1-trityl-1H-indazol-3-yl]carbamoyl}pyrrolidine-1-carboxylate [(XIII), R1=2-chloro-5-cyanophenyl, R2=tert-butyl pyrrolidine-1-carboxylate, R9=methanetriyltribenzene]

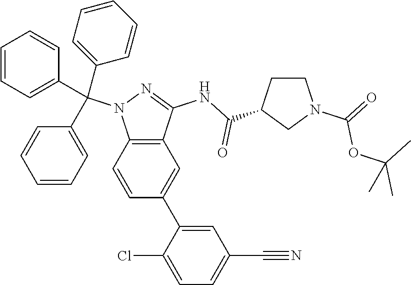 [SMILES: CC(C)(C)OC(=O)N1CC[C@@H](C(=O)Nc2nn(C(c3ccccc3)(c3ccccc3)c3ccccc3)c3ccc(-c4cc(C#N)ccc4Cl)cc23)C1]

Starting from 3-(3-amino-1-trityl-1H-indazol-5-yl)-4-chlorobenzonitrile and (3R)-1-(tert-butoxycarbonyl)pyrrolidine-3-carboxylic acid. Flash column chromatography (Hexane/EtOAc 70:30). Obtained 0.088 g (94% of yield). ¹H NMR (500 MHz, DMSO-d₆) δ ppm 1.37 (s, 9H), 2.00-2.17 (m, 2H), 3.15-3.30 (m, 2H), 3.36-3.45 (m, 1H), 3.51 (d, J=8.69 Hz, 1H), 6.47 (d, J=9.00 Hz, 1H), 7.17 (d, J=8.69 Hz, 1H), 7.24 (d, 6H), 7.28-7.31 (m, 3H), 7.34-7.37 (m, 6H), 7.76 (d, J=8.39 Hz, 1H), 7.81-7.92 (m, 3H), 10.72 (d, J=5.80 Hz, 1H). HRMS (ESI) calcd for C43H39ClN5O3Na [M+H+Na]⁺730.2555, found 730.2572.

### tert-Butyl (2S,5R)-5-{[5-(2,6-dichlorophenyl)-1-trityl-1H-indazol-3-yl]carbamoyl}-2-methylpiperidine-1-carboxylate [(XIII), R1=2,6-dichlorophenyl, R2=tert-butyl (2S)-2-methylpiperidine-1-carboxylate, R9=1,1′,1″-methanetriyltribenzene]

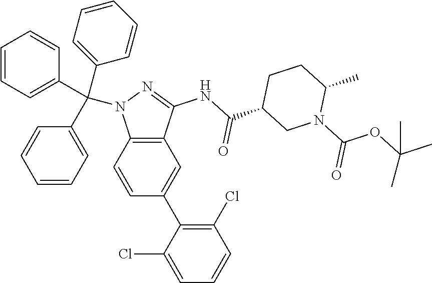 [SMILES: C[C@H]1CC[C@@H](C(=O)Nc2nn(C(c3ccccc3)(c3ccccc3)c3ccccc3)c3ccc(-c4c(Cl)cccc4Cl)cc23)CN1C(=O)OC(C)(C)C]

Starting from 5-(2,6-dichlorophenyl)-1-trityl-1H-indazol-3-ylamine and (3R,6S)-1-(tert-butoxycarbonyl)-6-methylpiperidine-3-carboxylic acid. Flash column chromatography (Hexane/EtOAc 80:20). Obtained 0.040 g (81% of yield). LCMS (ESI) m/z 745 (M+H)⁺.

### tert-Butyl (2R,5R)-5-{[5-(2,6-dichlorophenyl)-1-trityl-1H-indazol-3-yl]carbamoyl}-2-methylpiperidine-1-carboxylate [(XIII), R1=2,6-dichlorophenyl, R2=tert-butyl (2R)-2-methylpiperidine-1-carboxylate, R9=1,1′,1″-methanetriyltribenzene]

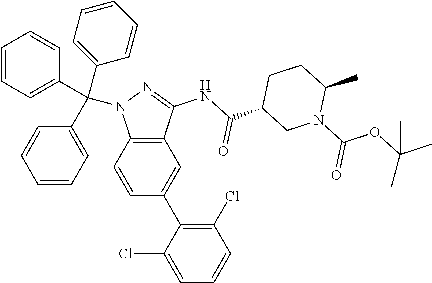 [SMILES: C[C@@H]1CC[C@@H](C(=O)Nc2nn(C(c3ccccc3)(c3ccccc3)c3ccccc3)c3ccc(-c4c(Cl)cccc4Cl)cc23)CN1C(=O)OC(C)(C)C]

Starting from 5-(2,6-dichlorophenyl)-1-trityl-1H-indazol-3-ylamine and (3R,6R)-1-(tert-butoxycarbonyl)-6-methylpiperidine-3-carboxylic acid. Flash column chromatography (Hexane/EtOAc 80:20). Obtained 0.061 g (86% of yield). LCMS (ESI) m/z 745 (M+H)⁺.

### tert-Butyl (2R,5R)-5-{[5-(2-chloro-5-cyanophenyl)-1-trityl-1H-indazol-3-yl]carbamoyl}-2-methylpiperidine-1-carboxylate [(XIII), R1=2-chloro-5-cyanophenyl, R2=tert-butyl (2R)-2-methylpiperidine-1-carboxylate, R9=1,1′,1″-methanetriyltribenzene]

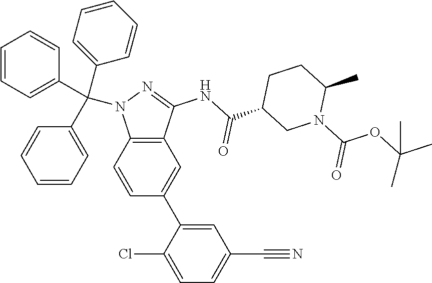 [SMILES: C[C@@H]1CC[C@@H](C(=O)Nc2nn(C(c3ccccc3)(c3ccccc3)c3ccccc3)c3ccc(-c4cc(C#N)ccc4Cl)cc23)CN1C(=O)OC(C)(C)C]

Starting from 3-(3-amino-1-trityl-1H-indazol-5-yl)-4-chlorobenzonitrile and (3R,6R)-1-(tert-butoxycarbonyl)-6-methylpiperidine-3-carboxylic acid. Flash column chromatography (Hexane/EtOAc 80:20). Obtained 0.075 g (83% of yield). LCMS (ESI) m/z 736 (M+H)⁺.

### Benzyl (2S,5R)-5-{[5-(2-chloro-5-cyanophenyl)-1-trityl-1H-indazol-3-yl]carbamoyl}-2-(trifluoromethyl)-piperidine-1-carboxylate [(XIII), R1=2-chloro-5-cyanophenyl, R2=benzyl (2S)-2-(trifluoromethyl)piperidine-1-carboxylate, R9=1,1′,1″-methanetriyltribenzene]

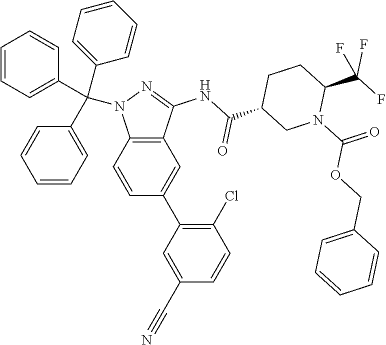 [SMILES: N#Cc1ccc(Cl)c(-c2ccc3c(c2)c(NC(=O)[C@@H]2CC[C@@H](C(F)(F)F)N(C(=O)OCc4ccccc4)C2)nn3C(c2ccccc2)(c2ccccc2)c2ccccc2)c1]

Starting from 3-(3-amino-1-trityl-1H-indazol-5-yl)-4-chlorobenzonitrile and (3R,6S)-1-[(benzyloxy)carbonyl]-6-(trifluoromethyl)piperidine-3-carboxylic acid. Flash column chromatography (Hexane/EtOAc 70:30). Obtained 0.090 g (70% of yield). LCMS (ESI) m/z 824 (M+H)⁺.

### tert-Butyl (3S)-3-{[5-(2-chloro-5-cyanophenyl)-1-trityl-1H-indazol-3-yl]carbamoyl}piperidine-1-carboxylate [(XIII), R1=2-chloro-5-cyanophenyl, R2=tert-butyl piperidine-1-carboxylate, R9=methanetriyltribenzene]

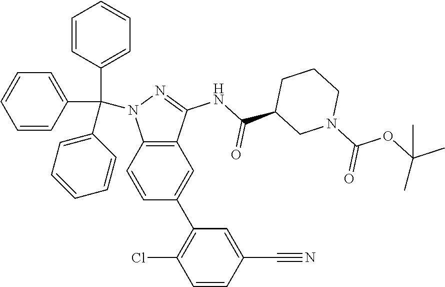 [SMILES: CC(C)(C)OC(=O)N1CCC[C@H](C(=O)Nc2nn(C(c3ccccc3)(c3ccccc3)c3ccccc3)c3ccc(-c4cc(C#N)ccc4Cl)cc23)C1]

Starting from 3-(3-amino-1-trityl-1H-indazol-5-yl)-4-chlorobenzonitrile and (3S)-1-(tert-butoxycarbonyl)piperidine-3-carboxylic acid. Flash column chromatography (Hexane/EtOAc 70:30). Obtained 0.180 g (92% of yield). ¹H NMR (500 MHz, DMSO-d₆) δ ppm 1.36 (s, 9H), 1.57-1.74 (m, 2H), 1.90-1.96 (m, 1H), 2.58 (m., 1H), 2.75-2.83 (m, 1H), 3.77-3.80 (m, 1H), 6.46 (d, J=8.85 Hz, 1H), 7.16 (d, J=8.69 Hz, 1H), 7.23 (d, J=7.63 Hz, 6H), 7.28-7.31 (m, 3H), 7.33-7.36 (m, 6H), 7.77 (d, J=8.24 Hz, 1H), 7.82-7.91 (m, 3H), 10.65 (s, 1H). HRMS (ESI) calcd for C44H41ClN5O3Na [M+H+Na]⁺744.2712, found 744.2720.

### tert-Butyl (2R)-2-{[5-(2-chloro-5-cyanophenyl)-1-trityl-1H-indazol-3-yl]carbamoyl}morpholine-4-carboxylate [(XIII), R1=2-chloro-5-cyanophenyl, R2=tert-butyl morpholine-4-carboxylate, R9=methanetriyltribenzene]

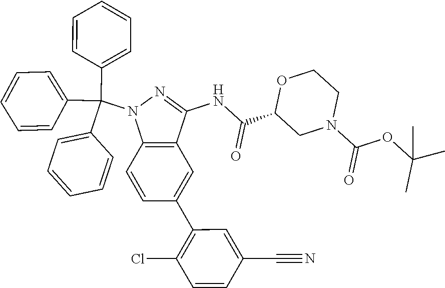 [SMILES: CC(C)(C)OC(=O)N1CCO[C@@H](C(=O)Nc2nn(C(c3ccccc3)(c3ccccc3)c3ccccc3)c3ccc(-c4cc(C#N)ccc4Cl)cc23)C1]

Starting from 3-(3-amino-1-trityl-1H-indazol-5-yl)-4-chlorobenzonitrile and (2R)-4-(tert-butoxycarbonyl)morpholine-2-carboxylic acid. Flash column chromatography (Hexane/EtOAc 70:30). Obtained 0.085 g (83% of yield). ¹H NMR (500 MHz, DMSO-d₆) δ ppm 1.38 (s, 9H), 2.97-3.05 (m, 2H), 3.51 (t, J=10.22 Hz, 1H), 3.67 (m, 1H), 3.87-4.06 (m, 2H), 4.16 (dd, J=9.99, 2.97 Hz, 1H), 6.47 (d, J=9.00 Hz, 1H), 7.19 (dd, J=8.92, 1.45 Hz, 1H), 7.23 (d, J=7.32 Hz, 6H), 7.28-7.31 (m, 3H), 7.33-7.36 (m, 6H), 7.76-7.81 (m, 2H), 7.84 (dd, J=8.24, 1.98 Hz, 1H), 7.88 (d, J=1.83 Hz, 1H), 10.36 (s, 1H). HRMS (ESI) calcd for C43H39ClN5O4Na [M+H+Na]⁺746.2504, found 746.2522.

### tert-Butyl 3-{[5-(2-chloro-5-cyanophenyl)-1-trityl-1H-indazol-3-yl]carbamoyl}azepane-1-carboxylate [(XIII), R1=2-chloro-5-cyanophenyl, R2=tert-butyl azepane-1-carboxylate, R9=1,1′,1″-methanetriyltribenzene]

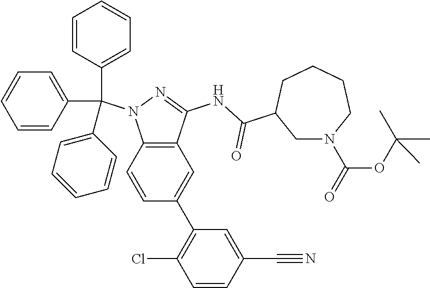 [SMILES: CC(C)(C)OC(=O)N1CCCCC(C(=O)Nc2nn(C(c3ccccc3)(c3ccccc3)c3ccccc3)c3ccc(-c4cc(C#N)ccc4Cl)cc23)C1]

Starting from 3-(3-amino-1-trityl-1H-indazol-5-yl)-4-chlorobenzonitrile and 1-(tert-butoxycarbonyl)azepane-3-carboxylic acid. Flash column chromatography (Hexane/EtOAc 70:30). Obtained 0.053 g (94% of yield). ¹H NMR (500 MHz, DMSO-d₆) δ ppm 1.37 (s, 10H), 6.46 (d, J=8.69 Hz, 1H), 7.16 (t, J=7.47 Hz, 1H), 7.24 (d, J=8.39 Hz, 6H), 7.28-7.31 (m, 3H), 7.33-7.38 (m, 6H), 7.73-7.90 (m, 4H), 10.61 (s, 1H). HRMS (ESI) calcd for C45H43ClN5O3Na [M+H+Na]⁺758.2868, found 758.2886.

### tert-Butyl 5-{[5-(2-chloro-5-cyanophenyl)-1-trityl-1H-indazol-3-yl]carbamoyl}-2,2-dimethylpiperidine-1-carboxylate [(XIII), R1=2-chloro-5-cyanophenyl, R2=tert-butyl 2,2-dimethylpiperidine-1-carboxylate, R9=1,1′,1″-methanetriyltribenzene]

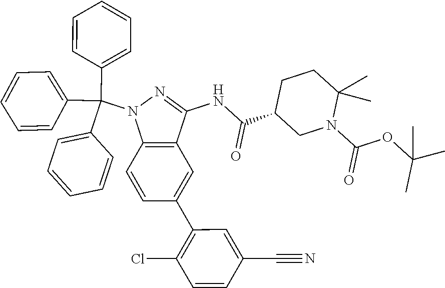 [SMILES: CC(C)(C)OC(=O)N1C[C@H](C(=O)Nc2nn(C(c3ccccc3)(c3ccccc3)c3ccccc3)c3ccc(-c4cc(C#N)ccc4Cl)cc23)CCC1(C)C]

Starting from 3-(3-amino-1-trityl-1H-indazol-5-yl)-4-chlorobenzonitrile and 1-(tert-butoxycarbonyl)-6,6-dimethyl-piperidine-3-carboxylic acid. Flash column chromatography (Hexane/EtOAc 80:20). Obtained 0.118 g (90% of yield). ¹H NMR (500 MHz, DMSO-d₆) δ ppm 1.28 (s, 9H), 1.31 (s, 3H), 1.37 (s, 3H), 1.50-1.67 (m, 2H), 1.69-1.93 (m, 2H), 2.81 (m, 1H), 3.39 (dd, J=13.27, 8.54 Hz, 1H), 3.75 (dt, J=13.54, 4.29 Hz, 1H), 6.46 (d, J=9.00 Hz, 1H), 7.16 (d, J=8.85 Hz, 1H), 7.23 (d, J=7.47 Hz, 6H), 7.28-7.31 (m, 3H), 7.33-7.36 (m, 6H), 7.75-7.79 (m, 1H), 7.82-7.87 (m, 3H), 10.60 (s, 1H). HRMS (ESI) calcd for C46H45ClN5O3Na [M+H+Na]⁺772.3025, found 772.3011.

### tert-Butyl 8-{[5-(2-chloro-5-cyanophenyl)-1-trityl-1H-indazol-3-yl]carbamoyl}-6-azaspiro[4.5]decane-6-carboxylate [(XIII), R1=2-chloro-5-cyanophenyl, R2=tert-butyl 6-azaspiro[4.5]decane-6-carboxylate, R9=1,1′,1″-methanetriyltribenzene]

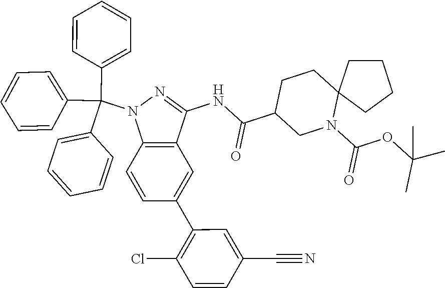 [SMILES: CC(C)(C)OC(=O)N1CC(C(=O)Nc2nn(C(c3ccccc3)(c3ccccc3)c3ccccc3)c3ccc(-c4cc(C#N)ccc4Cl)cc23)CCC12CCCC2]

Starting from 3-(3-amino-1-trityl-1H-indazol-5-yl)-4-chlorobenzonitrile and 6-(tert-butoxycarbonyl)-6-azaspiro[4.5]decane-8-carboxylic acid. Flash column chromatography (Hexane/EtOAc 80:20). Obtained 0.110 g (93% of yield). ¹H NMR (500 MHz, DMSO-d₆) δ ppm 1.33 (s, 9H), 1.46-1.85 (m, 10H), 2.07-2.21 (m, 1H), 2.72 (m, 1H), 3.25-3.32 (m, 1H), 3.83-3.89 (m, 1H), 6.46 (d, J=9.00 Hz, 1H), 7.16 (d, J=9.00 Hz, 1H), 7.23 (d, J=7.47 Hz, 6H), 7.28-7.31 (m, 3H), 7.33-7.36 (m, 6H), 7.77 (d, J=8.24 Hz, 1H), 7.83-7.88 (m, 3H), 10.64 (s, 1H). HRMS (ESI) calcd for C48H47ClN5O3Na [M+H+Na]⁺798.3181, found 798.3217.

### tert-Butyl 3-{[5-(2-chloro-5-cyanophenyl)-1-trityl-1H-indazol-3-yl]carbamoyl}-1-azaspiro[5.5]undecane-1-carboxylate [(XIII), R1=2-chloro-5-cyanophenyl, R2=tert-butyl 1-azaspiro[5.5]undecane-1-carboxylate, R9=1,1′,1″-methanetriyltribenzene]

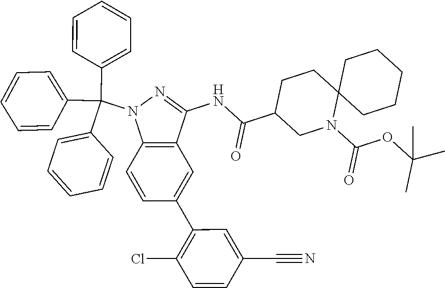 [SMILES: CC(C)(C)OC(=O)N1CC(C(=O)Nc2nn(C(c3ccccc3)(c3ccccc3)c3ccccc3)c3ccc(-c4cc(C#N)ccc4Cl)cc23)CCC12CCCCC2]

Starting from 3-(3-amino-1-trityl-1H-indazol-5-yl)-4-chlorobenzonitrile and 1-(tert-butoxycarbonyl)-1-azaspiro[5.5]undecane-3-carboxylic acid. Flash column chromatography (Hexane/EtOAc 80:20). Obtained 0.320 g (85% of yield). ¹H NMR (500 MHz, DMSO-d₆) δ ppm 1.28 (s, 9H), 1.32-1.90 (m, 10H), 2.41 (t, J=10.14 Hz, 1H), 2.81 (m, 1H), 3.53 (dd, J=13.88, 8.39 Hz, 1H), 3.76 (dd, J=13.65, 4.80 Hz, 1H), 6.46 (d, J=8.85 Hz, 1H), 7.16 (d, J=9.00 Hz, 1H), 7.24 (d, J=7.47 Hz, 6H), 7.27-7.32 (m, 3H), 7.32-7.37 (m, 6H), 7.76 (d, J=8.85 Hz, 1H), 7.81-7.87 (m, 3H), 10.60 (s, 1H). HRMS (ESI) calcd for C49H49ClN5O3Na [M+H+Na]⁺812.2520, found 812.2515.

### tert-Butyl 4-{[5-(2-chloro-5-cyanophenyl)-1-trityl-1H-indazol-3-yl]carbamoyl}-2-azabicyclo[2.2.2]octane-2-carboxylate [(XIII), R1=2-chloro-5-cyanophenyl, R2=tert-butyl 2-azabicyclo[2.2.2]octane-2-carboxylate, R9=1,1′,1″-methanetriyltribenzene]

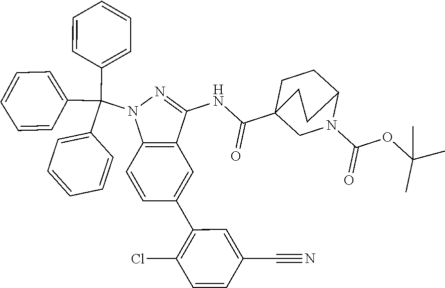 [SMILES: CC(C)(C)OC(=O)N1CC2(C(=O)Nc3nn(C(c4ccccc4)(c4ccccc4)c4ccccc4)c4ccc(-c5cc(C#N)ccc5Cl)cc34)CCC1CC2]

Starting from 3-(3-amino-1-trityl-1H-indazol-5-yl)-4-chlorobenzonitrile and 2-(tert-butoxycarbonyl)-2-azabicyclo[2.2.2]octane-4-carboxylic acid. Flash column chromatography (Hexane/EtOAc 70:30). Obtained 0.166 g (92% of yield). ¹H NMR (500 MHz, DMSO-d₆) δ ppm 1.39 (m, 9H), 1.56-2.11 (m, 8H), 3.23-3.43 (m, 3H), 6.45 (d, J=9.00 Hz, 1H), 7.15 (m, 1H), 7.20-7.25 (m, 6H), 7.28-7.31 (m, 3H), 7.33-7.38 (m, 6H), 7.61 (d, J=4.88 Hz, 1H), 7.76 (d, J=8.39 Hz, 1H), 7.84 (m, 1H), 7.88 (d, J=1.98 Hz, 1H), 10.10 (d, J=11.13 Hz, 1H). HRMS (ESI) calcd for C46H43ClN5O3Na [M+H+Na]⁺770.3049, found 770.3044.

### N-[5-(2-Chloro-5-cyanophenyl)-1-trityl-1H-indazol-3-yl]-1-azabicyclo[2.2.2]octane-3-carboxamide [(XIII), R1=2-chloro-5-cyanophenyl, R2=1-azabicyclo[2.2.2]octane, R9=1,1′,1″-methanetriyltribenzene]

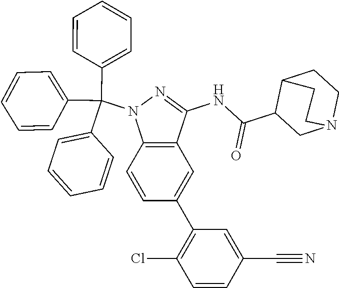 [SMILES: N#Cc1ccc(Cl)c(-c2ccc3c(c2)c(NC(=O)C2CN4CCC2CC4)nn3C(c2ccccc2)(c2ccccc2)c2ccccc2)c1]

Starting from 3-(3-amino-1-trityl-1H-indazol-5-yl)-4-chlorobenzonitrile and 1-azabicyclo[2.2.2]octane-3-carboxylic acid. Flash column chromatography (DCM/MeOH/33% aqueous ammonia 90:10:1). Obtained 0.077 g (81% of yield). LCMS (ESI) m/z 648 (M+H)⁺.

### N-[5-(2,6-Dichlorophenyl)-1-trityl-1H-indazol-3-yl]-4-(dimethylamino)cyclohexanecarboxamide [(XIII), R1=2,6-dichlorophenyl, R2=4-(dimethylamino)cyclohexane, R9=1,1′,1″-methanetriyltribenzene]

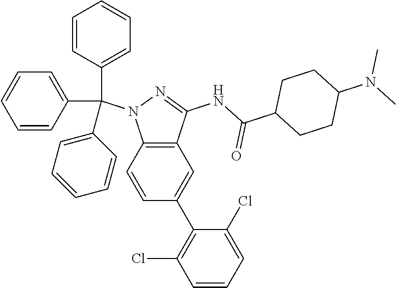 [SMILES: CN(C)C1CCC(C(=O)Nc2nn(C(c3ccccc3)(c3ccccc3)c3ccccc3)c3ccc(-c4c(Cl)cccc4Cl)cc23)CC1]

Starting from 5-(2,6-dichlorophenyl)-1-trityl-1H-indazol-3-ylamine and 4-(dimethylamino)cyclohexanecarboxylic acid. Flash column chromatography (DCM/MeOH 90:10). Obtained 0.144 g (89% of yield). LCMS (ESI) m/z 673 (M+H)⁺.

### 9H-Fluoren-9-ylmethyl 4-{[5-(2-chloro-5-cyanophenyl)-1-trityl-1H-indazol-3-yl]carbamoyl}piperidine-1-carboxylate [(XIII), R1=2-Chloro-5-cyanophenyl, R2=9H-fluoren-9-ylmethyl piperidine-1-carboxylate]

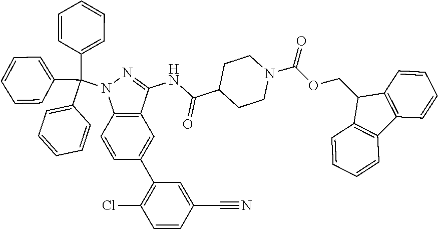 [SMILES: N#Cc1ccc(Cl)c(-c2ccc3c(c2)c(NC(=O)C2CCN(C(=O)OCC4c5ccccc5-c5ccccc54)CC2)nn3C(c2ccccc2)(c2ccccc2)c2ccccc2)c1]

Starting from 3-(3-amino-1-trityl-1H-indazol-5-yl)-4-chloro-benzonitrile and 1-[(9H-fluoren-9-ylmethoxy)carbonyl]-piperidine-4-carboxylic acid. Flash column chromatography (DCM/MeOH 92:8). Obtained 0.87 g (88% of yield). LCMS (ESI) m/z 844 (M+H)⁺.

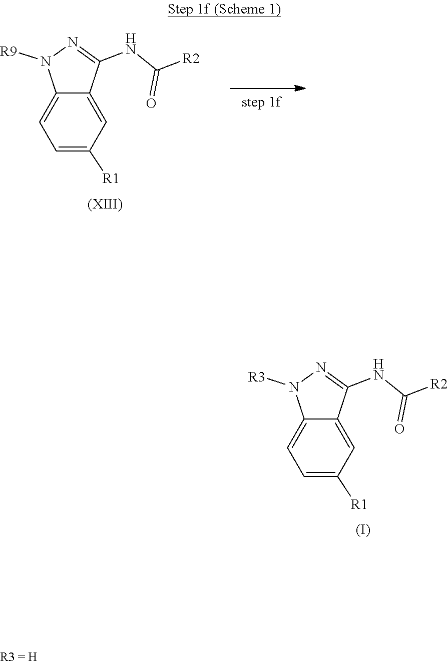

The compounds were prepared according to the same procedure reported for step 1c (Scheme 1).

### 1-Butyl-N-[5-(2-chloro-5-cyanophenyl)-1H-indazol-3-yl]piperidine-4-carboxamide hydrochloride (cpd 140) [(I), R1=2-chloro-5-cyanophenyl, R2=1-butylpiperidine, R3=H]

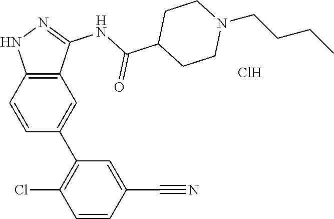 [SMILES: CCCCN1CCC(C(=O)Nc2n[nH]c3ccc(-c4cc(C#N)ccc4Cl)cc23)CC1 ; Cl]

Obtained 0.035 g (85% of yield). ¹H NMR (500 MHz, DMSO-d₆) δ ppm 0.92 (t, J=7.40 Hz, 3H), 1.32 (sxt, J=7.29 Hz, 2H), 1.65 (m, 2H), 1.86-1.99 (m, 2H), 2.09 (d, J=14.18 Hz, 2H), 2.74 (m, 1H), 2.87-2.99 (m, 2H), 3.03 (m, 2H), 3.55 (m, 2H), 7.43 (dd, J=8.69, 1.53 Hz, 1H), 7.55 (d, J=8.69 Hz, 1H), 7.82 (d, J=8.40 Hz, 1H), 7.85 (s, 1 H), 7.89 (dd, J=8.40, 2.00 Hz, 1H), 7.93 (d, J=1.98 Hz, 1H), 9.45 (br. s., 1H), 10.62 (s, 1H), 12.91 (br. s., 1H). HRMS (ESI) calcd for C24H27ClN5O [M+H]⁺ 436.1899, found 436.1904.

### N-[5-(2-Chloro-5-cyanophenyl)-1H-indazol-3-yl]-1-propylpiperidine-4-carboxamide hydrochloride (cpd 141) [(I), R1=2-chloro-5-cyanophenyl, R2=1-propylpiperidine, R3=H]

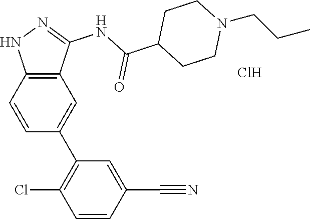 [SMILES: CCCN1CCC(C(=O)Nc2n[nH]c3ccc(-c4cc(C#N)ccc4Cl)cc23)CC1 ; Cl]

Obtained 0.037 g (88% of yield). ¹H NMR (500 MHz, DMSO-d₆) δ ppm 0.91 (t, J=7.32 Hz, 3H), 1.61-1.75 (m, 2H), 1.87-1.97 (m, 2H), 2.06-2.12 (m, 2H), 2.74 (m, 1H), 2.88-2.96 (m, 2H), 3.01 (m, 2H), 3.54 (m, 2H), 7.43 (dd, J=8.69, 1.37 Hz, 1H), 7.55 (d, J=8.69 Hz, 1H), 7.81 (d, J=8.39 Hz, 1H), 7.85 (s, 1H), 7.88 (dd, J=8.24, 1.98 Hz, 1H), 7.93 (d, J=1.98 Hz, 1H), 9.42 (br. s., 1H), 10.62 (s, 1H), 12.90 (br. s., 1H). HRMS (ESI) calcd for C23H25ClN5O [M+H]⁺ 422.1742, found 422.1751.

### N-[5-(2-Chloro-5-cyanophenyl)-1H-indazol-3-yl]-1-pentylpiperidine-4-carboxamide hydrochloride (cpd 142) [(I), R1=2-chloro-5-cyanophenyl, R2=1-pentylpiperidine, R3=H]

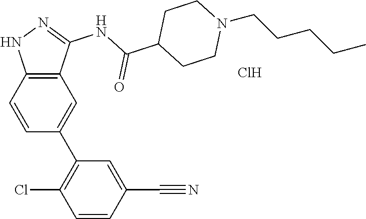 [SMILES: CCCCCN1CCC(C(=O)Nc2n[nH]c3ccc(-c4cc(C#N)ccc4Cl)cc23)CC1 ; Cl]

Obtained 0.043 g (86% of yield). ¹H NMR (500 MHz, DMSO-d₆) δ ppm 0.87-0.91 (m, 3H), 1.22-1.37 (m, 4H), 1.67 (m, 2H), 1.86-1.98 (m, 2H), 2.05-2.14 (m, 2H), 2.75 (m, 1H), 2.88-2.98 (m, 2H), 3.03 (m, 2H), 3.53 (m, 2H), 7.43 (dd, J=8.69, 1.37 Hz, 1H), 7.55 (d, J=8.69 Hz, 1H), 7.81 (d, J=8.39 Hz, 1H), 7.85 (s, 1H), 7.88 (dd, J=8.24, 1.98 Hz, 1H), 7.93 (d, J=1.98 Hz, 1H), 9.45 (br. s., 1H), 10.62 (s, 1H), 12.91 (br. s., 1H). HRMS (ESI) calcd for C25H29ClN5O [M+H]⁺ 450.2055, found 450.2062.

### N-[5-(2-Chloro-5-cyanophenyl)-1H-indazol-3-yl]-1-(propan-2-yl)piperidine-4-carboxamide hydrochloride (cpd 143) [(I), R1=2-chloro-5-cyanophenyl, R2=1-(propan-2-yl)piperidine, R3=H]

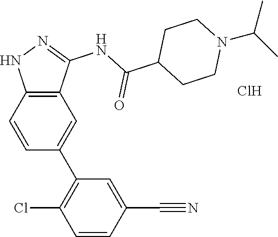 [SMILES: CC(C)N1CCC(C(=O)Nc2n[nH]c3ccc(-c4cc(C#N)ccc4Cl)cc23)CC1 ; Cl]

Obtained 0.040 g (99% of yield). ¹H NMR (500 MHz, DMSO-d₆) δ ppm 1.27 (d, J=6.71 Hz, 6H), 1.93-2.03 (m, 2H), 2.08-2.16 (m, 2H), 2.76 (m, 1H), 2.93-3.02 (m, 2H), 3.27 (m, 1H), 3.43-3.47 (m, 2H), 7.43 (dd, J=8.69, 1.53 Hz, 1H), 7.55 (d, J=8.54 Hz, 1H), 7.81-7.85 (m, 2H), 7.88 (dd, J=8.39, 2.13 Hz, 1H), 7.93 (d, J=1.83 Hz, 1H), 9.30 (br. s., 1H), 10.62 (s, 1H), 12.91 (br. s., 1H). HRMS (ESI) calcd for C23H25ClN5O [M+H]⁺ 422.1742, found 422.1747.

### N-[5-(2,6-Dichlorophenyl)-1H-indazol-3-yl]-4-(methylamino)cyclohexanecarboxamide hydrochloride (cpd 144) [(I), R1=2,6-dichlorophenyl, R2=4-(methylamino)cyclohexane, R3=H]

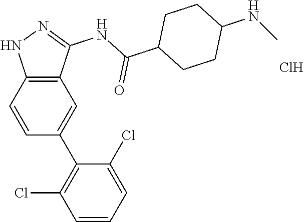 [SMILES: CNC1CCC(C(=O)Nc2n[nH]c3ccc(-c4c(Cl)cccc4Cl)cc23)CC1 ; Cl]

Obtained 0.025 g (86% of yield). ¹H NMR (500 MHz, DMSO-d₆) δ ppm 1.62-1.69 (m, 2H), 1.73-1.85 (m, 4H), 1.90-2.11 (m, 2H), 2.69 (m, 1H), 3.05 (m, 1H), 7.18 (dd, J=8.62, 1.60 Hz, 1H), 7.43 (t, J=7.78 Hz, 1H), 7.53 (d, J=8.54 Hz, 1H), 7.59 (d, J=8.08 Hz, 2H), 7.69 (s, 1H), 8.47 (br. s., 2H), 10.23-10.44 (m, 1H), 12.83 (br. s., 1H). HRMS (ESI) calcd for C21H23Cl2N4O [M+H]⁺ 417.1244, found 417.1253.

### N-[5-(2,6-Dichlorophenyl)-1H-indazol-3-yl]-3-(methylamino)cyclobutanecarboxamide hydrochloride (cpd 145) [(I), R1=2,6-dichlorophenyl, R2=3-(methylamino)cyclobutane, R3=H]

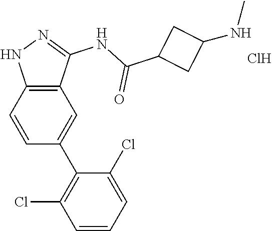 [SMILES: CNC1CC(C(=O)Nc2n[nH]c3ccc(-c4c(Cl)cccc4Cl)cc23)C1 ; Cl]

Obtained 0.015 g (86% of yield). ¹H NMR (500 MHz, DMSO-d₆) δ ppm 2.24-2.36 (m, 2H), 2.39-2.47 (m, 5H), 3.11 (t, J=8.39 Hz, 1H), 3.61-3.85 (m, 1H), 7.19 (dd, J=8.54, 1.53 Hz, 1H), 7.44 (dd, J=8.54, 7.78 Hz, 1H), 7.53 (d, J=8.54 Hz, 1H), 7.59 (d, J=7.93 Hz, 2H), 7.68-7.81 (m, 1H), 8.64-8.91 (m, 2H), 10.50-10.55 (m, 1H), 12.85 (br. s., 1H).). HRMS (ESI) calcd for C19H19Cl2N4O [M+H]⁺ 389.0931, found 389.0936.

### N-[5-(2-Chloro-5-cyanophenyl)-1H-indazol-3-yl]-3-(methylamino)cyclobutanecarboxamide hydrochloride (cpd 146) [(I), R1=2-chloro-5-cyanophenyl, R2=3-(methylamino)cyclobutane, R3=H]

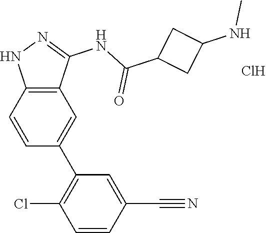 [SMILES: CNC1CC(C(=O)Nc2n[nH]c3ccc(-c4cc(C#N)ccc4Cl)cc23)C1 ; Cl]

Obtained 0.006 g (72% of yield). ¹H NMR (500 MHz, DMSO-d₆) δ ppm 2.30-2.38 (m, 2H), 2.42-2.47 (m, 5H), 3.13 (m, 1H), 3.57-3.69 (m, 1H), 7.43 (dd, J=8.62, 1.45 Hz, 1H), 7.55 (d, J=8.54 Hz, 1H), 7.81 (d, J=8.39 Hz, 1H), 7.88-7.91 (m, 2H), 7.93 (d, J=1.98 Hz, 1H), 8.63-8.98 (m, 2H), 10.56 (s, 1H), 12.90 (br. s., 1H). HRMS (ESI) calcd for C20H19ClN5O [M+H]⁺ 380.1273, found 380.1277.

### N-[5-(2-Chloro-5-cyanophenyl)-1H-indazol-3-yl]-3-(dimethylamino)cyclobutanecarboxamide hydrochloride (cpd 147) [(I), R1=2-chloro-5-cyanophenyl, R2=3-(dimethylamino)cyclobutane, R3=H]

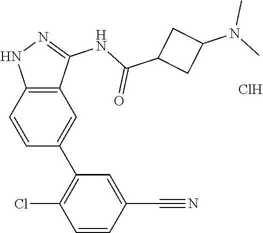 [SMILES: CN(C)C1CC(C(=O)Nc2n[nH]c3ccc(-c4cc(C#N)ccc4Cl)cc23)C1 ; Cl]

Obtained 0.018 g (78% of yield). 1H NMR (500 MHz, DMSO-d₆) δ ppm 2.38-2.46 (m, 4H), 2.67 (d, J=4.73 Hz, 6H), 3.05 (m, 1H), 3.59-3.71 (m, 1H), 7.43 (dd, J=8.69, 1.53 Hz, 1H), 7.55 (d, J=8.69 Hz, 1H), 7.81 (d, J=8.39 Hz, 1H), 7.87-7.91 (m, 2H,), 7.93 (d, J=1.98 Hz, 1H), 9.87 (br. s., 1H), 10.60 (s, 1H), 12.91 (br. s., 1H). HRMS (ESI) calcd for C21H21ClN5O [M+H]⁺ 394.1429, found 394.1429.

### N-[5-(2-Chloro-5-cyanophenyl)-1H-indazol-3-yl]-3-(propan-2-ylamino)cyclobutanecarboxamide hydrochloride (cpd 148) [(I), R1=2-chloro-5-cyanophenyl, R2=3-(propan-2-ylamino)cyclobutane, R3=H]

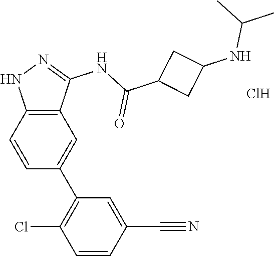 [SMILES: CC(C)NC1CC(C(=O)Nc2n[nH]c3ccc(-c4cc(C#N)ccc4Cl)cc23)C1 ; Cl]

Obtained 0.020 g (74% of yield). ¹H NMR (500 MHz, DMSO-d₆) δ ppm 1.22 (d, J=6.56 Hz, 6H), 2.42-2.51 (m, 2H), 3.12-3.32 (m, 2H), 3.77-3.88 (m, 1H), 7.47 (dd, J=8.69, 1.37 Hz, 1H), 7.58 (d, J=8.54 Hz, 1H), 7.85 (d, J=8.39 Hz, 1H), 7.92-7.97 (m, 3H), 8.74 (br. s., 2H), 10.61 (s, 1H), 12.93 (br. s., 1H). HRMS (ESI) calcd for C22H23ClN5O [M+H]⁺ 408.1586, found 408.1592.

### N-[5-(2-Chloro-5-cyanophenyl)-1H-indazol-3-yl]-3-(morpholin-4-yl)cyclobutanecarboxamide hydrochloride (cpd 149) [(I), R1=2-chloro-5-cyanophenyl, R2=3-(morpholin-4-yl)cyclobutane, R3=H]

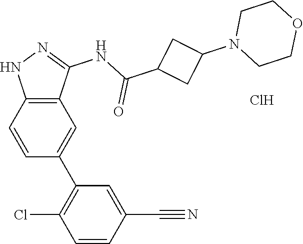 [SMILES: N#Cc1ccc(Cl)c(-c2ccc3[nH]nc(NC(=O)C4CC(N5CCOCC5)C4)c3c2)c1 ; Cl]

Obtained 0.029 g (83% of yield). ¹H NMR (500 MHz, DMSO-d₆) δ ppm 2.44-2.48 (m, 2H), 2.84-2.99 (m, 2H), 3.10 (m, 1H), 3.31-3.36 (m, 2H), 3.64 (t, J=11.67 Hz, 2H), 3.72 (m, 1H), 3.98 (d, J=10.83 Hz, 2H), 7.44 (dd, J=8.62, 1.45 Hz, 1H), 7.55 (d, J=8.69 Hz, 1H), 7.81 (d, J=8.24 Hz, 1H), 7.87-7.91 (m, 2H), 7.93 (d, J=1.98 Hz, 1H), 10.16 (d, J=6.41 Hz, 1H), 10.61 (s, 1H), 12.91 (br. s., 1H). HRMS (ESI) calcd for C23H23ClN5O2 [M+H]⁺ 436.1535, found 436.1543.

### N-[5-(2-Chloro-5-cyanophenyl)-1H-indazol-3-yl]-3-[(2,2,2-trifluoroethyl)amino]cyclobutanecarboxamide hydro-chloride (cpd 150) [(I), R1=2-chloro-5-cyanophenyl, R2=3-[(2,2,2-trifluoroethyl)amino]cyclobutane, R3=H]

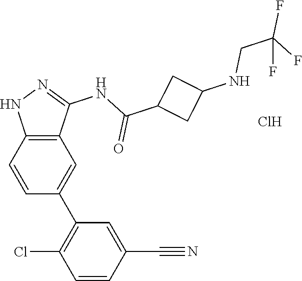 [SMILES: N#Cc1ccc(Cl)c(-c2ccc3[nH]nc(NC(=O)C4CC(NCC(F)(F)F)C4)c3c2)c1 ; Cl]

Obtained 0.017 g (55% of yield). ¹H NMR (500 MHz, DMSO-d₆) δ ppm 2.40-2.48 (m, 2H), 3.13 (quin, J=8.66 Hz, 1H), 3.77 (m, 1H), 4.00 (br. s., 2H), 7.43 (dd, J=8.77, 1.45 Hz, 1H), 7.55 (d, J=8.69 Hz, 1H), 7.81 (d, J=8.39 Hz, 1H), 7.87-7.96 (m, 3H), 9.99 (br. s., 1H), 10.54 (s, 1H), 12.90 (br. s., 1H).). HRMS (ESI) calcd for C21H18ClF3N5O [M+H]⁺ 448.1147, found 448.1160.

### N-[5-(2-Chloro-5-cyanophenyl)-1H-indazol-3-yl]-3-[(2-fluoroethyl)amino]cyclobutanecarboxamide hydrochloride (cpd 151) [(I), R1=2-chloro-5-cyanophenyl, R2=3-[(2-fluoroethyl)amino]cyclobutane, R3=H]

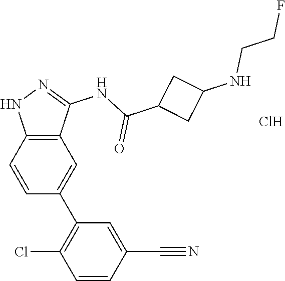 [SMILES: N#Cc1ccc(Cl)c(-c2ccc3[nH]nc(NC(=O)C4CC(NCCF)C4)c3c2)c1 ; Cl]

Obtained 0.022 g (81% of yield). ¹H NMR (500 MHz, DMSO-d₆) δ ppm 2.37-2.49 (m, 2H), 3.07-3.16 (m, 1H), 3.17-3.27 (m, 2H), 3.67-3.80 (m, 1H), 4.60-4.77 (m, 2H), 7.43 (dd, J=8.54, 1.22 Hz, 1H), 7.55 (d, J=8.69 Hz, 1H), 7.81 (d, J=8.39 Hz, 1H), 7.87-7.91 (m, 2H), 7.93 (d, J=1.83 Hz, 1H), 9.14 (br. s., 2H), 10.56 (s, 1H), 12.90 (br. s., 1H). HRMS (ESI) calcd for C21H20ClFN50 [M+H]⁺ 412.1335, found 412.1343.

### N-[5-(2-Chloro-5-cyanophenyl)-1H-indazol-3-yl]-3-(ethylamino)cyclobutanecarboxamide hydrochloride (cpd 152) [(I), R1=2-chloro-5-cyanophenyl, R2=3-(ethylamino)cyclobutane, R3=H]

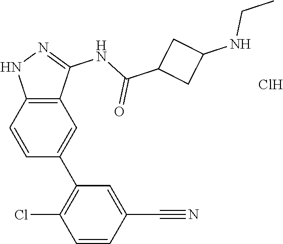 [SMILES: CCNC1CC(C(=O)Nc2n[nH]c3ccc(-c4cc(C#N)ccc4Cl)cc23)C1 ; Cl]

Obtained 0.014 g (62% of yield). ¹H NMR (500 MHz, DMSO-d₆) δ ppm 1.15 (t, J=7.24 Hz, 3H), 2.33-2.41 (m, 2H), 2.44-2.48 (m, 2H), 2.84 (m, 2H), 3.14 (m, 1H), 3.63-3.75 (m, 1H), 7.43 (dd, J=8.77, 1.30 Hz, 1H), 7.55 (d, J=8.69 Hz, 1H), 7.81 (d, J=8.39 Hz, 1H), 7.88-7.90 (m, 2H), 7.93 (d, J=1.83 Hz, 1H), 10.57 (s, 1H), 12.90 (br. s., 1H). HRMS (ESI) calcd for C21H21ClN5O [M+H]⁺ 394.1429, found 394.1434.

### N-[5-(2-Chloro-5-cyanophenyl)-1H-indazol-3-yl]-3-(cyclobutylamino)cyclobutanecarboxamide hydrochloride (cpd 153) [(I), R1=2-chloro-5-cyanophenyl, R2=3-(cyclobutylamino)cyclobutane, R3=H]

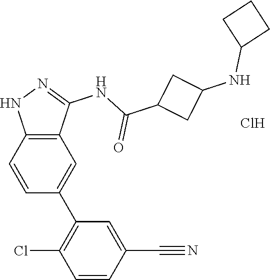 [SMILES: N#Cc1ccc(Cl)c(-c2ccc3[nH]nc(NC(=O)C4CC(NC5CCC5)C4)c3c2)c1 ; Cl]

Obtained 0.031 g (70% of yield). ¹H NMR (500 MHz, DMSO-d₆) δ ppm 1.65-1.86 (m, 2H), 2.03-2.20 (m, 4H), 2.32-2.49 (m, 4H), 3.14 (m, 1H), 3.55-3.77 (m, 2H), 7.43 (dd, J=8.69, 1.37 Hz, 1H), 7.55 (d, J=8.54 Hz, 1H), 7.81 (d, J=8.39 Hz, 1H), 7.87-7.91 (m, 2H), 7.93 (d, J=1.98 Hz, 1H), 9.07 (br. s., 2H), 10.50 (s, 1H), 12.90 (br. s., 1H). HRMS (ESI) calcd for C23H23ClN5O [M+H]⁺ 420.1586, found 420.1585.

### N-[5-(2-Chloro-5-cyanophenyl)-1H-indazol-3-yl]-3-(cyclopentylamino)cyclobutanecarboxamide hydrochloride (cpd 154) [(I), R1=2-chloro-5-cyanophenyl, R2=3-(cyclopentylamino)cyclobutane, R3=H]

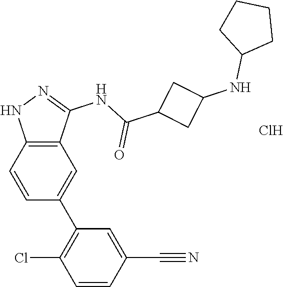 [SMILES: N#Cc1ccc(Cl)c(-c2ccc3[nH]nc(NC(=O)C4CC(NC5CCCC5)C4)c3c2)c1 ; Cl]

Obtained 0.035 g (76% of yield). ¹H NMR (500 MHz, DMSO-d₆) δ ppm 1.47-1.58 (m, 4H), 1.63-1.70 (m, 3H), 1.88-1.96 (m, 3H), 2.37-2.44 (m, 2H), 3.14 (t, J=8.39 Hz, 1H), 3.37 (m, 1H), 3.81 (m, 1H), 7.43 (dd, J=8.69, 1.22 Hz, 1H), 7.55 (d, J=8.69 Hz, 1H), 7.81 (d, J=8.39 Hz, 1H), 7.87-7.91 (m, 2H), 7.93 (d, J=1.98 Hz, 1H), 8.86 (br. s., 1H), 10.58 (s, 1H), 12.90 (br. s., 1H). HRMS (ESI) calcd for C24H25ClN5O [M+H]⁺ 434.1498, found 434.1504.

### N-[5-(2-Chloro-5-cyanophenyl)-1H-indazol-3-yl]-3-(cyclopropylamino)cyclobutanecarboxamide hydrochloride (cpd 155) [(I), R1=2-chloro-5-cyanophenyl, R2=3-(cyclopropylamino)cyclobutane, R3=H]

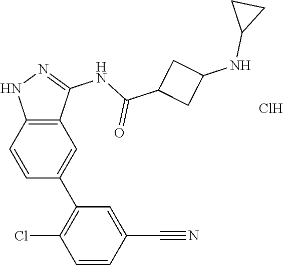 [SMILES: N#Cc1ccc(Cl)c(-c2ccc3[nH]nc(NC(=O)C4CC(NC5CC5)C4)c3c2)c1 ; Cl]

Obtained 0.025 g (79% of yield). ¹H NMR (500 MHz, DMSO-d₆) δ ppm 0.68-0.80 (m, 4H), 2.37-2.45 (m, 2H), 2.54-2.63 (m, 3H), 3.17 (t, J=8.46 Hz, 1H), 3.81 (m, 1H), 7.43 (dd, J=8.69, 1.37 Hz, 1H), 7.55 (d, J=8.54 Hz, 1H), 7.81 (d, J=8.24 Hz, 1H), 7.87-7.91 (m, 2H), 7.92 (m, 1H), 9.06 (br. s., 1H), 10.57 (s, 1H), 12.90 (br. s., 1H).). HRMS (ESI) calcd for C22H21ClN5O [M+H]⁺ 406.1429, found 406.1433.

### N-[5-(2,6-Dichlorophenyl)-1H-indazol-3-yl]tetrahydro-2H-pyran-4-carboxamide (cpd 156) [(I), R1=2,6-dichlorophenyl, R2=tetrahydro-2H-pyran, R3=H]

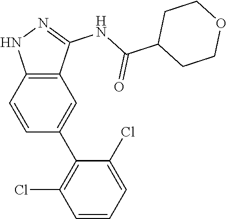 [SMILES: O=C(Nc1n[nH]c2ccc(-c3c(Cl)cccc3Cl)cc12)C1CCOCC1]

Obtained 0.022 g (67% of yield). 1H NMR (500 MHz, DMSO-d₆) δ ppm 1.61-1.70 (m, 2H), 1.71-1.78 (m, 2H), 2.66-2.76 (m, 1H), 3.29-3.38 (m, 2H), 3.89 (dd, J=11.44, 2.14 Hz, 2H), 7.18 (dd, J=8.69, 1.53 Hz, 1H), 7.44 (dd, J=8.54, 7.78 Hz, 1H), 7.52 (d, J=8.69 Hz, 1H), 7.59 (d, J=7.93 Hz, 2H), 7.69 (s, 1H), 10.40 (s, 1H), 12.80 (br. s., 1H). HRMS (ESI) calcd for C19H18Cl2N3O2 [M+H]⁺ 390.0771, found 390.0772.

### N-[5-(2,6-Dichlorophenyl)-1H-indazol-3-yl]piperidine-3-carboxamide hydrochloride (cpd 157) [(I), R1=2,6-dichlorophenyl, R2=piperidine, R3=H]

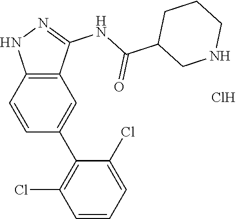 [SMILES: O=C(Nc1n[nH]c2ccc(-c3c(Cl)cccc3Cl)cc12)C1CCCNC1 ; Cl]

Obtained 0.018 g (83% of yield). ¹H NMR (500 MHz, DMSO-d₆) δ ppm 1.63-1.86 (m, 2H), 2.07 (br. s., 2H), 2.90 (d, J=9.15 Hz, 1H), 2.93-3.01 (m, 1H), 3.04-3.12 (m, 1H), 3.15 (d, J=11.59 Hz, 1H), 3.28-3.36 (m, 1H), 7.20 (dd, J=8.69, 1.53 Hz, 1H), 7.44 (dd, J=8.54, 7.78 Hz, 1H), 7.55 (d, J=8.69 Hz, 1H), 7.60 (d, J=7.93 Hz, 2H), 7.71 (d, J=0.76 Hz, 1H), 8.53-8.81 (m, 2H), 10.72 (s, 1H), 12.89 (br. s., 1H). HRMS (ESI) calcd for C19H19Cl2N4O [M+H]⁺389.0931, found 389.0931.

### 1-Cyclopropyl-N-[5-(2,6-dichlorophenyl)-1H-indazol-3-yl]piperidine-4-carboxamide hydrochloride (cpd 158) [(I), R1=2,6-dichlorophenyl, R2=1-cyclopropylpiperidine, R3=H]

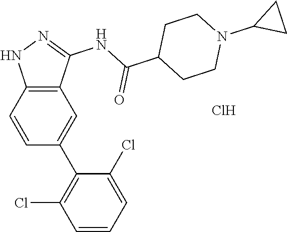 [SMILES: O=C(Nc1n[nH]c2ccc(-c3c(Cl)cccc3Cl)cc12)C1CCN(C2CC2)CC1 ; Cl]

Obtained 0.041 g (95% of yield). ¹H NMR (500 MHz, DMSO-d₆) δ ppm 0.67-0.89 (m, 2H), 0.92-1.05 (m, 2H), 1.81-1.98 (m, 2H), 2.00-2.13 (m, 2H), 2.67-2.80 (m, 1H), 3.10-3.24 (m, 2H), 3.35 (d, J=6.86 Hz, 1H), 3.53-3.59 (m, 2H), 7.19 (dd, J=8.54, 1.37 Hz, 1H), 7.42-7.46 (m, 1H), 7.54 (d, J=8.69 Hz, 1H), 7.57-7.62 (m, 2H), 7.66-7.71 (m, 1H), 9.40 (d, J=8.85 Hz, 1H), 10.59 (s, 1H), 12.87 (br. s., 1H). HRMS (ESI) calcd for C22H23Cl2N4O [M+H]⁺ 429.1244, found 429.1241.

### N-[5-(2,6-Dichlorophenyl)-1H-indazol-3-yl]-1-phenylpiperidine-4-carboxamide hydrochloride (cpd 159) [(I), R1=2,6-dichlorophenyl, R2=1-phenylpiperidine, R3=H]

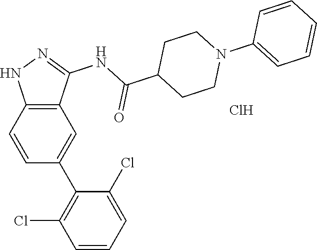 [SMILES: O=C(Nc1n[nH]c2ccc(-c3c(Cl)cccc3Cl)cc12)C1CCN(c2ccccc2)CC1 ; Cl]

Obtained 0.052 g (93% of yield). ¹H NMR (500 MHz, DMSO-d₆) δ ppm 2.09 (d, J=13.12 Hz, 4H), 2.83 (br. s., 1H), 3.71 (d, J=11.90 Hz, 3H), 7.20 (dd, J=8.69, 1.37 Hz, 1H), 7.42-7.49 (m, 6H), 7.55 (d, J=8.54 Hz, 1H), 7.60 (d, J=8.08 Hz, 2H), 7.70 (s, 1H), 10.59 (br. s., 1H), 12.87 (br. s., 1H). HRMS (ESI) calcd for C25H23Cl2N4O [M+H]⁺ 465.1244, found 465.1243.

### N-[5-(2,6-Dichlorophenyl)-1H-indazol-3-yl]-1-azabicyclo[2.2.2]octane-4-carboxamide (cpd 160) [(I), R1=2,6-dichlorophenyl, R2=1-azabicyclo[2.2.2]octane, R3=H]

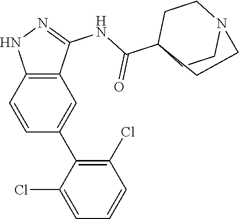 [SMILES: O=C(Nc1n[nH]c2ccc(-c3c(Cl)cccc3Cl)cc12)C12CCN(CC1)CC2]

Purified by flash column chromatography (DCM/MeOH/33% aqueous ammonia 80:20:2). Obtained 0.050 g (92% of yield). 1H NMR (500 MHz, DMSO-d₆) δ ppm 1.71-1.80 (m, 6H), 2.74-2.85 (m, 6H), 7.18 (dd, J=8.62, 1.60 Hz, 1H), 7.42-7.45 (m, 1H), 7.48 (s, 1H), 7.54 (dd, J=8.62, 0.69 Hz, 1H), 7.59 (d, J=8.24 Hz, 2H), 9.75 (s, 1H), 12.86 (s, 1H). HRMS (ESI) calcd for C21H21Cl2N4O [M+H]⁺ 415.1087, found 415.1091.

### N-[5-(2-Chloro-6-fluorophenyl)-1H-indazol-3-yl]-1-methylpiperidine-3-carboxamide hydrochloride (cpd 161) [(I), R1=2-chloro-6-fluorophenyl, R2=1-methylpiperidine, R3=H]

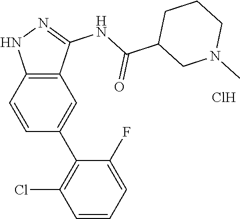 [SMILES: CN1CCCC(C(=O)Nc2n[nH]c3ccc(-c4c(F)cccc4Cl)cc23)C1 ; Cl]

Obtained 0.024 g (81% of yield). ¹H NMR (500 MHz, DMSO-d₆) δ ppm 1.49-1.58 (m, 1H), 1.73-1.94 (m, 2H), 2.11-2.14 (m, 1H), 2.73-2.79 (m, 3H), 3.00-3.15 (m, 3H), 3.54-3.60 (m, 2H), 7.29 (d, J=8.54 Hz, 1H), 7.33-7.40 (m, 1H), 7.43-7.50 (m, 2H), 7.52-7.59 (m, 1H), 7.71-7.89 (m, 1H), 9.98 (br. s., 1H), 10.67-11.00 (m, 1H), 12.79-12.99 (m, 1H). HRMS (ESI) calcd for C20H21ClFN4O [M+H]⁺ 387.1383, found 387.1380.

### N-[5-(2,6-Dichlorophenyl)-1H-indazol-3-yl]-2-[(2S)-pyrrolidin-2-yl]acetamide hydrochloride (cpd 162) [(I), R1=2,6-dichlorophenyl, R2=(2R)-2-methylpyrrolidine, R3=H]

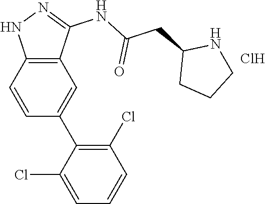 [SMILES: O=C(C[C@@H]1CCCN1)Nc1n[nH]c2ccc(-c3c(Cl)cccc3Cl)cc12 ; Cl]

Obtained 0.018 g (64% of yield). ¹H NMR (500 MHz, DMSO-d₆) δ ppm 1.55-1.69 (m, 1H), 1.77-1.90 (m, 1H), 1.91-2.03 (m, 1H), 2.11-2.20 (m, 1H), 2.86-2.94 (m, 1H), 2.97-3.03 (m, 1H), 3.11-3.21 (m, 2H), 3.76-3.87 (m, 1H), 7.23 (dd, J=8.62, 1.60 Hz, 1H), 7.45-7.50 (m, 1H), 7.58 (d, J=8.54 Hz, 1H), 7.61-7.67 (m, 2H), 7.84 (s, 1H), 8.65 (br. s., 1H), 9.02 (br. s., 1H), 10.81 (s, 1H), 12.91 (br. s., 1H). HRMS (ESI) calcd for C19H19Cl2N4O [M+H]⁺ 389.0931, found 389.0932.

### N-[5-(2,6-Dichlorophenyl)-1H-indazol-3-yl]-2-(piperidin-4-yl)acetamide hydrochloride (cpd 163) [(I), R1=2,6-dichlorophenyl, R2=4-methylpiperidine, R3=H]

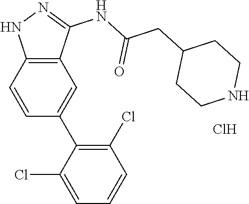 [SMILES: O=C(CC1CCNCC1)Nc1n[nH]c2ccc(-c3c(Cl)cccc3Cl)cc12 ; Cl]

Obtained 0.035 g (92% of yield). ¹H NMR (500 MHz, DMSO-d₆) δ ppm 1.32-1.48 (m, 2H), 1.83 (d, J=12.81 Hz, 2H), 1.99-2.11 (m, 1H), 2.34-2.38 (m, 2H), 2.78-2.92 (m, 2H), 3.24 (d, J=12.81 Hz, 2H), 7.19 (dd, J=8.62, 1.45 Hz, 1H), 7.44 (dd, J=8.46, 7.70 Hz, 1H), 7.53 (d, J=8.54 Hz, 1H), 7.60 (d, J=8.08 Hz, 2H), 7.73 (s, 1H), 8.36 (d, J=9.15 Hz, 1H), 8.62 (br. s., 1H), 10.47-10.56 (m, 1H), 12.82 (br. s., 1H). HRMS (ESI) calcd for C20H21Cl2N4O [M+H]⁺ 403.1087, found 403.1085.

### trans-4-(Aminomethyl)-N-[5-(2,6-dichlorophenyl)-1H-indazol-3-yl]cyclohexanecarboxamide hydrochloride (cpd 164) [(I), R1=2,6-dichlorophenyl, R2=1-cyclohexylmethanamine, R3=H]

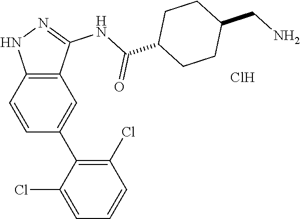

Obtained 0.027 g (73% of yield). ¹H NMR (500 MHz, DMSO-d₆) δ ppm 0.91-1.15 (m, 2H), 1.42 (qd, J=12.76, 3.05 Hz, 2H), 1.54 (dd, J=6.63, 3.89 Hz, 1H), 1.83 (d, J=11.13 Hz, 2H), 1.92 (d, J=10.83 Hz, 2H), 2.42 (t, J=12.05 Hz, 1H), 2.64-2.72 (m, 2H), 7.18 (dd, J=8.62, 1.45 Hz, 1H), 7.44 (dd, J=8.46, 7.70 Hz, 1H), 7.52 (d, J=8.54 Hz, 1H), 7.58-7.63 (m, 2H), 7.69 (s, 1H), 7.78 (br. s., 3H), 10.37 (s, 1H), 12.79 (br. s., 1H). HRMS (ESI) calcd for C21H22Cl2N4O [M+H]⁺ 417.1244, found 417.1243.

### N-[5-(2-Chloro-6-fluorophenyl)-1H-indazol-3-yl]piperidine-3-carboxamide hydrochloride (cpd 165) [(I), R1=2-chloro-6-fluorophenyl, R2=piperidine, R3=H]

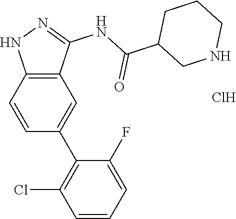 [SMILES: O=C(Nc1n[nH]c2ccc(-c3c(F)cccc3Cl)cc12)C1CCCNC1 ; Cl]

Obtained 0.056 g (88% of yield). ¹H NMR (500 MHz, DMSO-d₆) δ ppm 1.63-1.87 (m, 2H), 2.01-2.14 (m, 1H), 2.82-3.02 (m, 2H), 3.04-3.22 (m, 2H), 3.32 (br. s., 2H), 7.29 (d, J=8.39 Hz, 1H), 7.31-7.40 (m, 1H), 7.43-7.49 (m, 2H), 7.55 (d, J=8.69 Hz, 1H), 7.78 (s, 1H), 8.49-8.79 (m, 2H), 10.73 (s, 1H), 12.91 (br. s., 1H). HRMS (ESI) calcd for C19H19ClFN4O [M+H]⁺ 373.1226, found 373.1230.

### N-[5-(2,6-Dichlorophenyl)-1H-indazol-3-yl]-4-(dimethylamino)cyclohexanecarboxamide hydrochloride (cpd 166) [(I), R1=2,6-dichlorophenyl, R2=4-(dimethylamino)cyclohexane, R3=H]

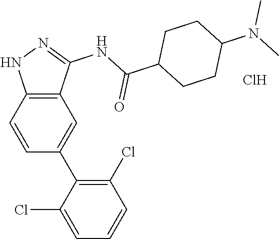 [SMILES: CN(C)C1CCC(C(=O)Nc2n[nH]c3ccc(-c4c(Cl)cccc4Cl)cc23)CC1 ; Cl]

Obtained 0.018 g (71% of yield). ¹H NMR (500 MHz, DMSO-d₆) δ ppm 1.42-1.65 (m, 3H), 1.78-1.88 (m, 2H), 2.00-2.16 (m, 3H), 2.69 (dd, J=14, 03, 5.03 Hz, 6H), 2.77 (m, 1H), 3.15 (m, 1H), 7.19 (ddd, J=8.58, 5.76, 1.53 Hz, 1H), 7.44 (t, J=8.39, 1H), 7.55 (m, 1H), 7.60 (d, J=8.16, Hz, 2H), 7.68 (s, 1H), 9.91 (br. s., 1H), 10.38-10.43 (m, 1H), 12.84 (br. .s, 1H). HRMS (ESI) calcd for C22H25Cl2N4O [M+H]⁺ 431.14, found 431.14.

### (3R)-N-[5-(2,6-Dichlorophenyl)-1H-indazol-3-yl]pyrrolidine-3-carboxamide hydrochloride (cpd 167) [(I), R1=2,6-dichlorophenyl, R2=pyrrolidine, R3=H]

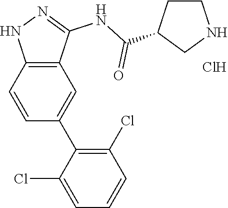 [SMILES: O=C(Nc1n[nH]c2ccc(-c3c(Cl)cccc3Cl)cc12)[C@@H]1CCNC1 ; Cl]

Obtained 0.045 g (94% of yield). ¹H NMR (500 MHz, DMSO-d₆) δ ppm 2.03-2.17 (m, 1H), 2.29 (dd, J=13.27, 6.86 Hz, 1H), 3.22 (dq, J=13.38, 6.77 Hz, 2H), 7.19 (dd, J=8.54, 1.53 Hz, 1H), 7.44 (t, J=8.39, Hz, 1H), 7.55 (d, J=8.69 Hz, 1H), 7.59 (d, J=8.08 Hz, 2H), 7.75 (s, 1H), 8.79-9.17 (m, 2H), 10.81 (s, 1H), 12.90 (br. s., 1H). HRMS (ESI) calcd for C18H17Cl2N4O [M+H]⁺ 375.0774, found 375.0771.

### (3S)-N-[5-(2,6-Dichlorophenyl)-1H-indazol-3-yl]pyrrolidine-3-carboxamide hydrochloride (cpd 168) [(I), R1=2,6-dichlorophenyl, R2=pyrrolidine, R3=H]

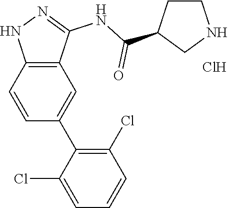 [SMILES: O=C(Nc1n[nH]c2ccc(-c3c(Cl)cccc3Cl)cc12)[C@H]1CCNC1 ; Cl]

Obtained 0.029 g (88% of yield). ¹H NMR (500 MHz, DMSO-d₆) δ ppm 2.03-2.17 (m, 1H), 2.29 (dd, J=13.27, 6.86 Hz, 1H), 3.22 (dq, J=13.38, 6.77 Hz, 2H), 7.19 (dd, J=8.54, 1.53 Hz, 1H), 7.44 (t, J=8.39, Hz, 1H), 7.55 (d, J=8.69 Hz, 1H), 7.59 (d, J=8.08 Hz, 2H), 7.75 (s, 1H), 8.79-9.17 (m, 2H), 10.81 (s, 1H), 12.90 (br. s., 1H). HRMS (ESI) calcd for C18H17Cl2N4O [M+H]⁺ 375.0774, found 375.0779.

### (3R)-N-[5-(2,6-Dichlorophenyl)-1H-indazol-3-yl]piperidine-3-carboxamide hydrochloride (cpd 169) [(I), R1=2,6-dichlorophenyl, R2=piperidine, R3=H]

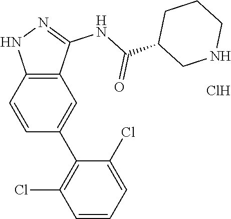 [SMILES: O=C(Nc1n[nH]c2ccc(-c3c(Cl)cccc3Cl)cc12)[C@@H]1CCCNC1 ; Cl]

Obtained 0.071 g (89% of yield). ¹H NMR (500 MHz, DMSO-d₆) δ ppm 1.57-1.90 (m, 3H), 2.06-2.10 (m, 1H), 2.83-2.92 (m, 1H), 2.94-3.02 (m, 1H), 3.09 (d, J=10.07 Hz, 1H), 3.15 (d, J=13.27 Hz, 1H), 3.33 (d, J=13.57 Hz, 1H), 7.19 (dd, J=8.62, 1.45 Hz, 1H), 7.44 (t, J=8.54, Hz, 1H), 7.55 (d, J=8.69 Hz, 1H), 7.60 (d, J=8.08 Hz, 2H), 7.70 (s, 1H), 8.54-8.82 (m, 2H), 10.72 (s, 1H), 12.89 (br. s., 1H). HRMS (ESI) calcd for C19H19Cl2N4O [M+H]⁺ 389.0931, found 389.0930.

### (3S)-N-[5-(2,6-Dichlorophenyl)-1H-indazol-3-yl]piperidine-3-carboxamide hydrochloride (cpd 170) [(I), R1=2,6-dichlorophenyl, R2=piperidine, R3=H]

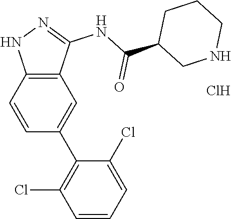 [SMILES: O=C(Nc1n[nH]c2ccc(-c3c(Cl)cccc3Cl)cc12)[C@H]1CCCNC1 ; Cl]

Obtained 0.031 g (38% of yield). ¹H NMR (500 MHz, DMSO-d₆) δ ppm 1.57-1.90 (m, 3H), 2.06-2.10 (m, 1H), 2.83-2.92 (m, 1H), 2.94-3.02 (m, 1H), 3.09 (d, J=10.07 Hz, 1H), 3.15 (d, J=13.27 Hz, 1H), 3.33 (d, J=13.57 Hz, 1H), 7.19 (dd, J=8.62, 1.45 Hz, 1H), 7.44 (t, J=8.54, Hz, 1H), 7.55 (d, J=8.69 Hz, 1H), 7.60 (d, J=8.08 Hz, 2H), 7.70 (s, 1H), 8.54-8.82 (m, 2H), 10.72 (s, 1H), 12.89 (br. s., 1H). HRMS (ESI) calcd for C19H19Cl2N4O [M+H]⁺ 389.0931, found 389.0934.

### N-[5-(2-Chloro-5-cyanophenyl)-1H-indazol-3-yl]piperidine-3-carboxamide hydrochloride (cpd 171) [(I), R1=2-chloro-5-cyanophenyl, R2=piperidine, R3=H]

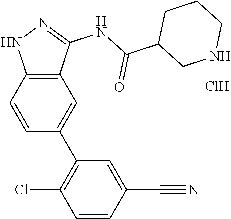 [SMILES: N#Cc1ccc(Cl)c(-c2ccc3[nH]nc(NC(=O)C4CCCNC4)c3c2)c1 ; Cl]

Obtained 0.022 g (77% of yield). ¹H NMR (500 MHz, DMSO-d₆) δ ppm 1.65-1.88 (m, 3H), 2.03-2.15 (m, 1H), 2.85-3.02 (m, 2H), 3.09 (m, 1H), 3.14-3.23 (m, 1H), 3.31-3.34 (m, 1H), 7.43 (dd, J=8.62, 1.60 Hz, 1H), 7.56 (d, J=8.54 Hz, 1H), 7.80-7.84 (m, 1H), 7.85 (s, 1H), 7.88-7.90 (m, 1H), 7.92 (d, J=1.98 Hz, 1H), 8.55-8.77 (m, 2H), 10.74 (s, 1H), 12.93 (br. s., 1H). HRMS (ESI) calcd for C20H19ClN5O [M+H]⁺ 380.1273, found 380.1278.

### (3R)-N-[5-(2-Chloro-6-fluorophenyl)-1H-indazol-3-yl]piperidine-3-carboxamide hydrochloride (cpd 172) [(I), R1=2-chloro-6-fluorophenyl, R2=piperidine, R3=H]

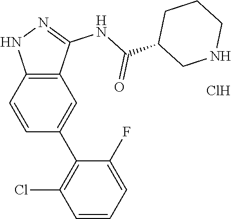 [SMILES: O=C(Nc1n[nH]c2ccc(-c3c(F)cccc3Cl)cc12)[C@@H]1CCCNC1 ; Cl]

Obtained 0.033 g (90% of yield). ¹H NMR (500 MHz, DMSO-d₆) δ ppm 1.64-1.86 (m, 3H), 2.03-2.14 (m, 1H), 2.90 (d, J=9.61 Hz, 1H), 2.95-3.01 (m, 1H), 3.09 (m, 1H), 3.15 (d, J=10.98 Hz, 1H), 3.33 (d, J=10.52 Hz, 1H), 7.29 (d, J=8.69 Hz, 1H), 7.35 (m, 1H), 7.43-7.50 (m, 2H), 7.55 (d, J=8.69 Hz, 1H), 7.78 (s, 1H), 8.56-8.83 (m, 2H), 10.73 (s, 1H), 12.91 (br. s., 1H). HRMS (ESI) calcd for C19H19ClFN4O [M+H]⁺ 373.1226, found 373.1228.

### (3S)-N-[5-(2-Chloro-6-fluorophenyl)-1H-indazol-3-yl]piperidine-3-carboxamide hydrochloride (cpd 173) [(I), R1=2-chloro-6-fluorophenyl, R2=piperidine, R3=H]

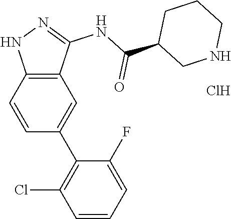 [SMILES: O=C(Nc1n[nH]c2ccc(-c3c(F)cccc3Cl)cc12)[C@H]1CCCNC1 ; Cl]

Obtained 0.029 g (88% of yield). ¹H NMR (500 MHz, DMSO-d₆) δ ppm 1.64-1.86 (m, 3H), 2.03-2.14 (m, 1H), 2.90 (d, J=9.61 Hz, 1H), 2.95-3.01 (m, 1H), 3.09 (m, 1H), 3.15 (d, J=10.98 Hz, 1H), 3.33 (d, J=10.52 Hz, 1H), 7.29 (d, J=8.69 Hz, 1H), 7.35 (m, 1H), 7.43-7.50 (m, 2H), 7.55 (d, J=8.69 Hz, 1H), 7.78 (s, 1H), 8.56-8.83 (m, 2H), 10.73 (s, 1H), 12.91 (br. s., 1H). HRMS (ESI) calcd for C19H19ClFN4O [M+H]⁺ 373.1226, found 373.1219.

### (3R)-N-[5-(2-Chloro-5-cyanophenyl)-1H-indazol-3-yl]piperidine-3-carboxamide hydrochloride (cpd 174) [(I), R1=2-chloro-5-cyanophenyl, R2=piperidine, R3=H]

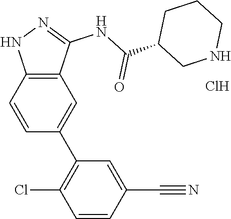 [SMILES: N#Cc1ccc(Cl)c(-c2ccc3[nH]nc(NC(=O)[C@@H]4CCCNC4)c3c2)c1 ; Cl]

Obtained 0.013 g (89% of yield). ¹H NMR (500 MHz, DMSO-d₆) δ ppm 1.65-1.88 (m, 3H), 2.03-2.15 (m, 1H), 2.85-3.02 (m, 2H), 3.09 (m, 1H), 3.14-3.23 (m, 1H), 3.31-3.34 (m, 1H), 7.43 (dd, J=8.62, 1.60 Hz, 1H), 7.56 (d, J=8.54 Hz, 1H), 7.80-7.84 (m, 1H), 7.85 (s, 1H), 7.88-7.90 (m, 1H), 7.92 (d, J=1.98 Hz, 1H), 8.55-8.77 (m, 2H), 10.74 (s, 1H), 12.93 (br. s., 1H). HRMS (ESI) calcd for C20H19ClN5O [M+H]⁺ 380.1273, found 380.1274.

### N-[5-(2,6-Dichlorophenyl)-1H-indazol-3-yl]-b-alaninamide hydrochloride (cpd 175) [(I), R1=2,6-dichlorophenyl, R2=ethylamine, R3=H]

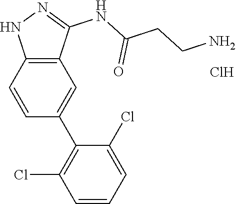 [SMILES: NCCC(=O)Nc1n[nH]c2ccc(-c3c(Cl)cccc3Cl)cc12 ; Cl]

Obtained 0.018 g (57% of yield). ¹H NMR (500 MHz, DMSO-d₆) δ ppm 2.79 (t, J=6.63 Hz, 2H), 3.09 (m, 2H), 7.19 (dd, J=8.62, 1.30 Hz, 1H), 7.44 (dd, J=8.39, 7.78 Hz, 1H), 7.54 (d, J=8.54 Hz, 1H), 7.60 (d, J=8.08 Hz, 2H), 7.77 (br. s., 3H), 7.81 (s, 1H), 10.72 (s, 1H), 12.85 (br. s., 1H). HRMS (ESI) calcd for C16H15Cl2N4O [M+H]⁺ 349.0618, found 349.0617.

### (3R)-N-[5-(2-Chloro-5-cyanophenyl)-1H-indazol-3-yl]pyrrolidine-3-carboxamide hydrochloride (cpd 176) [(I), R1=2-chloro-5-cyanophenyl, R2=pyrrolidine, R3=H]

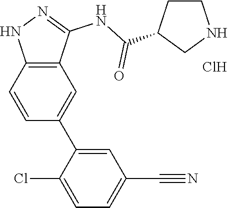 [SMILES: N#Cc1ccc(Cl)c(-c2ccc3[nH]nc(NC(=O)[C@@H]4CCNC4)c3c2)c1 ; Cl]

Obtained 0.047 g (98% of yield). ¹H NMR (500 MHz, DMSO-d₆) δ ppm 2.08-2.34 (m, 2H), 2.27-2.32 (m, 2H), 3.19-3.26 (m, 3H), 7.43 (dd, J=8.69, 1.53 Hz, 1H), 7.56 (d, J=8.69 Hz, 1H), 7.81 (d, J=8.24 Hz, 1H), 7.88-7.92 (m, 3H), 8.85-9.11 (m, 2H), 10.83 (s, 1H), 12.94 (br. s., 1H). HRMS (ESI) calcd for C19H17ClN5O [M+H]⁺ 366.1116, found 366.1120.

### (3R,6S)-N-[5-(2,6-Dichlorophenyl)-1H-indazol-3-yl]-6-methylpiperidine-3-carboxamide trifluoroacetate (cpd 177) [(1), R1=2,6-dichlorophenyl, R2=(2S)-2-methylpiperidine, R3=H]

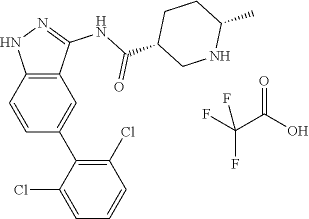

Obtained 0.005 g (30% of yield). ¹H NMR (500 MHz, DMSO-d₆) δ ppm 1.20 (d, J=6.56 Hz, 3H), 1.45-1.51 (m, 1H), 1.77-1.82 (m, 1H), 1.92-1.96 (m, 1H), 2.03-2.10 (m, 1H), 3.02 (t, J=4.35 Hz, 1H), 3.07-3.16 (m, 1H), 3.24 (m, 1H), 7.20 (dd, J=8.54, 1.53 Hz, 1H), 7.42-7.47 (m, 1H), 7.55 (d, J=8.54 Hz, 1H), 7.60 (d, J=7.93 Hz, 2H), 7.82 (s, 1H), 8.79 (d, J=10.07 Hz, 1H), 10.80 (s, 1H), 12.91 (s, 1H). HRMS (ESI) calcd for C20H21C12N4O [M+H]⁺403.1087, found 403.1092.

### (3R,6R)-N-[5-(2,6-Dichlorophenyl)-1H-indazol-3-yl]-6-methylpiperidine-3-carboxamide trifluoroacetate (cpd 178) [(I), R1=2,6-dichlorophenyl, R2=(2R)-2-methylpiperidine, R3=H]

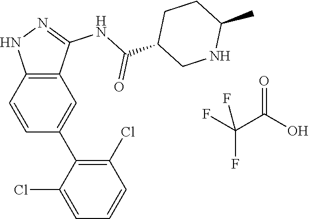

Obtained 0.010 g (49% of yield). ¹H NMR (500 MHz, DMSO-d₆) δ ppm 1.21 (d, J=6.41 Hz, 3H), 1.40-1.48 (m, 1H), 1.67 (dd, J=12.51, 3.05 Hz, 1H), 1.91 (d, J=12.81 Hz, 1H), 2.13 (d, J=12.35 Hz, 1H), 2.79-2.91 (m, 1H), 3.02-3.11 (m, 1H), 3.15 (m, 1H), 7.20 (dd, J=8.62, 1.45 Hz, 1H), 7.44 (dd, J=8.39, 7.78 Hz, 1H), 7.54 (d, J=8.69 Hz, 1H), 7.60 (d, J=8.08 Hz, 2H), 7.69 (s, 1H), 8.37 (d, J=10.22 Hz, 1H), 8.77 (d, J=10.52 Hz, 1H), 10.71 (s, 1H), 12.88 (s, 1H). HRMS (ESI) calcd for C20H21C12N4O [M+H]⁺ 403.1087, found 403.1086.

### (3R,6R)-N-[5-(2-Chloro-5-cyanophenyl)-1H-indazol-3-yl]-6-methylpiperidine-3-carboxamide trifluoroacetate (cpd 179) [(I), R1=2-chloro-5-cyanophenyl, R2=(2R)-2-methylpiperidine, R3=H]

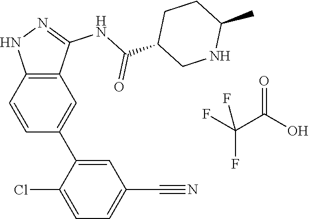

Obtained 0.012 g (54% of yield). ¹H NMR (500 MHz, DMSO-d₆) δ ppm 1.22 (d, J=6.41 Hz, 3H), 1.38-1.52 (m, 1H), 1.69 (dd, J=12.81, 3.36 Hz, 1H), 1.92 (d, J=14.18 Hz, 1H), 2.14 (d, J=11.44 Hz, 1H), 2.87 (m, 1H), 3.04-3.11 (m, 1H), 3.17 (m, 1H), 3.46 (d, J=11.13 Hz, 1H), 7.43 (dd, J=8.69, 1.53 Hz, 1H), 7.55 (d, J=8.54 Hz, 1H), 7.81-7.84 (m, 2H), 7.88-7.91 (m, 2H), 7.92 (d, J=1.83 Hz, 1H), 8.39 (d, J=11.29 Hz, 1H), 8.79 (d, J=10.52 Hz, 1H), 10.74 (s, 1H), 12.92 (s, 1H). HRMS (ESI) calcd for C21H21ClN5O [M+H]⁺ 394.1429, found 394.1432.

### (3R,6S)-N-[5-(2-Chloro-5-cyanophenyl)-1H-indazol-3-yl]-6-(trifluoromethyl)piperidine-3-carboxamide (cpd 180) [(1), R1=2-chloro-5-cyanophenyl, R2=(2S)-2-(trifluoromethyl)piperidine, R3=H]

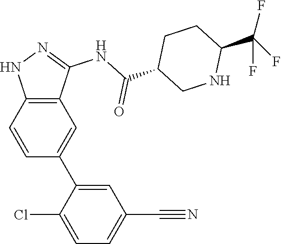 [SMILES: N#Cc1ccc(Cl)c(-c2ccc3[nH]nc(NC(=O)[C@@H]4CC[C@@H](C(F)(F)F)NC4)c3c2)c1]

Purified by flash column chromatography (DCM/MeOH/33% aqueous ammonia 80:20:2). Obtained 0.014 g (22% of yield). ¹H NMR (500 MHz, DMSO-d₆) δ ppm 1.30-1.43 (m, 1H), 1.58-1.74 (m, 1H), 1.83 (dd, J=12.51, 2.29 Hz, 1H), 2.01-2.10 (m, 1H), 2.56-2.69 (m, 2H), 3.16-3.24 (m, 2H), 7.41 (d, J=8.69 Hz, 1H), 7.53 (d, J=8.69 Hz, 1H), 7.80-7.83 (m, 2H), 7.87 (dd, J=8.24, 1.98 Hz, 1H), 7.93 (d, J=1.83 Hz, 1H), 10.46 (s, 1H), 12.84 (s, 1H). HRMS (ESI) calcd for C21H18ClF3N5O [M+H]⁺ 448.1147, found 448.1160.

### (3S)-N-[5-(2-Chloro-5-cyanophenyl)-1H-indazol-3-yl]piperidine-3-carboxamide hydrochloride (cpd 181) [(I), R1=2-chloro-5-cyanophenyl, R2=piperidine, R3=H]

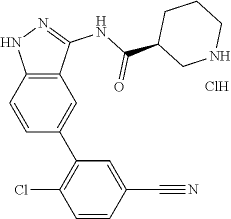 [SMILES: N#Cc1ccc(Cl)c(-c2ccc3[nH]nc(NC(=O)[C@H]4CCCNC4)c3c2)c1 ; Cl]

Obtained 0.105 g (99% of yield). ¹H NMR (500 MHz, DMSO-d₆) δ ppm 1.65-1.88 (m, 3H), 2.03-2.15 (m, 1H), 2.85-3.02 (m, 2H), 3.09 (m, 1H), 3.14-3.23 (m, 1H), 3.31-3.34 (m, 1H), 7.43 (dd, J=8.62, 1.60 Hz, 1H), 7.56 (d, J=8.54 Hz, 1H), 7.80-7.84 (m, 1H), 7.85 (s, 1H), 7.88-7.90 (m, 1H), 7.92 (d, J=1.98 Hz, 1H), 8.55-8.77 (m, 2H), 10.74 (s, 1H), 12.93 (br. s., 1H). HRMS (ESI) calcd for C20H19ClN5O [M+H]⁺ 380.1273, found 380.1276.

### (2R)-N-[5-(2-Chloro-5-cyanophenyl)-1H-indazol-3-yl]morpholine-2-carboxamide hydrochloride (cpd 182) [(I), R1=2-chloro-5-cyanophenyl, R2=morpholine]

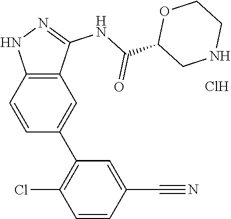 [SMILES: N#Cc1ccc(Cl)c(-c2ccc3[nH]nc(NC(=O)[C@H]4CNCCO4)c3c2)c1 ; Cl]

Obtained 0.038 g (94% of yield). ¹H NMR (500 MHz, DMSO-d₆) δ ppm 3.05-3.25 (m, 3H), 3.49 (d, J=11.59 Hz, 1H), 3.83-3.91 (m, 1H), 4.11 (d, J=13.12 Hz, 1H), 4.54 (dd, J=10.52, 2.75 Hz, 1H), 7.45 (dd, J=8.69, 1.53 Hz, 1H), 7.58 (d, J=8.69 Hz, 1H), 7.80-7.73 (m, 2H), 7.88 (dd, J=8.24, 1.98 Hz, 1H), 7.92 (d, J=1.98 Hz, 1H), 9.07-9.37 (m, 2H), 10.54 (s, 1H), 13.02 (br. s., 1H). HRMS (ESI) calcd for C19H17ClN5O2 [M+H]⁺ 382.1066, found 382.1067.

### N-[5-(2-Chloro-5-cyanophenyl)-1H-indazol-3-yl]azepane-3-carboxamide hydrochloride (cpd 183) [(I), R1=2-chloro-5-cyanophenyl, R2=azepane, R3=H]

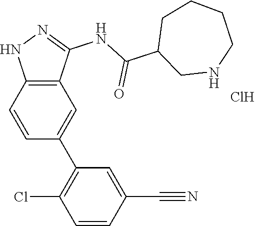 [SMILES: N#Cc1ccc(Cl)c(-c2ccc3[nH]nc(NC(=O)C4CCCCNC4)c3c2)c1 ; Cl]

Obtained 0.021 g (72% of yield). ¹H NMR (500 MHz, DMSO-d₆) δ ppm 1.56-1.90 (m, 4H), 2.09-2.21 (m, 1H), 3.01-3.17 (m, 3H), 3.29-3.37 (m, 3H), 7.43 (dd, J=8.62, 1.45 Hz, 1H), 7.55 (d, J=8.85 Hz, 1H), 7.81 (d, J=8.39 Hz, 1H), 7.87 (s, 1H), 7.88 (dd, J=8.24, 1.98 Hz, 1H), 7.92 (d, J=1.98 Hz, 1H), 8.65-8.97 (m, 2H), 10.71 (m, 1H), 12.93 (br. s., 1H). HRMS (ESI) calcd for C21H21ClN5O [M+H]⁺ 394.1429, found 394.1427.

### N-[5-(2-Chloro-5-cyanophenyl)-1H-indazol-3-yl]-6,6-dimethylpiperidine-3-carboxamide hydrochloride (cpd 184) [(I), R1=2-chloro-5-cyanophenyl, R2=2,2-dimethylpiperidine, R3=H]

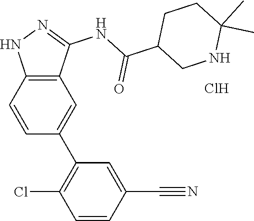 [SMILES: CC1(C)CCC(C(=O)Nc2n[nH]c3ccc(-c4cc(C#N)ccc4Cl)cc23)CN1 ; Cl]

Obtained 0.055 g (81% of yield). ¹H NMR (500 MHz, DMSO-d₆) δ ppm 1.29 (s, 3H), 1.35 (s, 3H), 1.60-1.69 (m, 1H), 1.70-1.76 (m, 1H), 1.76-1.85 (m, 1H), 2.02-2.06 (m, 1H), 2.93 (m, 1H), 3.08-3.21 (m, 1H), 3.30 (d, J=11.59 Hz, 1H), 7.43 (dd, J=8.69, 1.37 Hz, 1H), 7.55 (d, J=8.69 Hz, 1H), 7.81 (d, J=8.39 Hz, 1H), 7.87 (s, 1H), 7.88 (dd, J=8.39, 2.14 Hz, 1H), 7.92 (d, J=1.98 Hz, 1H), 8.71-8.98 (m, 2H), 10.75 (s, 1H), 12.93 (br. s., 1H). HRMS (ESI) calcd for C22H23ClN5O [M+H]⁺ 408.1586, found 408.1586.

### N-[5-(2-Chloro-5-cyanophenyl)-1H-indazol-3-yl]-6-azaspiro[4.5]decane-8-carboxamide hydrochloride (cpd 185) [(I), R1=2-chloro-5-cyanophenyl, R2=6-azaspiro[4.5]decane, R3=H]

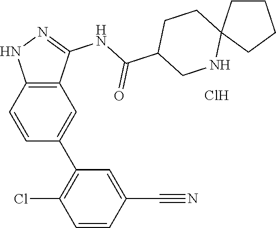 [SMILES: N#Cc1ccc(Cl)c(-c2ccc3[nH]nc(NC(=O)C4CCC5(CCCC5)NC4)c3c2)c1 ; Cl]

Obtained 0.072 g (88% of yield). ¹H NMR (500 MHz, DMSO-d₆) δ ppm 1.56-1.87 (m, 12H), 2.05-2.10 (m, 1H), 2.95 (m, 1H), 3.07-3.09 (m, 1H), 3.33-3.35 (m, 1H), 7.41 (dd, J=8.69, 1.52 Hz, 1H), 7.54 (d, J=8.69 Hz, 1H), 7.81 (d, J=8.24 Hz, 1H), 7.87 (s, 1H), 7.88 (dd, J=8.39, 2.13 Hz, 1H), 7.92 (d, J=1.83 Hz, 1H), 8.79 (d, J=10.07 Hz, 1H), 9.05 (br. s., 1H), 10.76 (s, 1H), 12.93 (br. s., 1H). HRMS (ESI) calcd for C24H25ClN5O [M+H]⁺ 434.1742, found 434.20.

### N-[5-(2-Chloro-5-cyanophenyl)-1H-indazol-3-yl]-1-azaspiro[5.5]undecane-3-carboxamide hydrochloride (cpd 186) [(I), R1=2-chloro-5-cyanophenyl, R2=1-azaspiro[5.5]undecane, R3=H]

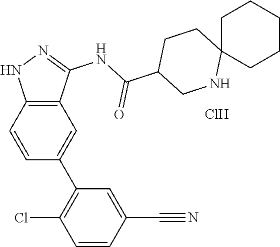 [SMILES: N#Cc1ccc(Cl)c(-c2ccc3[nH]nc(NC(=O)C4CCC5(CCCCC5)NC4)c3c2)c1 ; Cl]

Obtained 0.125 g (72% of yield). ¹H NMR (500 MHz, DMSO-d₆) δ ppm 1.23-1.64 (m, 9H), 1.72-1.80 (m, 2H), 1.91-2.12 (m, 3H), 2.96 (m, 1H), 3.14-3.21 (m, 1H), 3.24-3.29 (m, 1H), 7.41 (dd, J=8.85, 1.68 Hz, 1H), 7.54 (d, J=8.54 Hz, 1H), 7.81 (d, J=8.39 Hz, 1H), 7.87 (s, 1H), 7.88 (dd, J=8.24, 1.98 Hz, 1H), 7.91 (d, J=1.83 Hz, 1H), 8.68 (d, J=11.74 Hz, 1H), 8.95 (t, J=10.07 Hz, 1H), 10.75 (s, 1H), 12.93 (br. s., 1H). HRMS (ESI) calcd for C25H27ClN5O [M+H]⁺ 448.1899, found 448.1889.

### N-[5-(2-Chloro-5-cyanophenyl)-1H-indazol-3-yl]-2-azabicyclo[2.2.2]octane-4-carboxamide hydrochloride (cpd 187) [(I), R1=2-chloro-5-cyanophenyl, R2=2-azabicyclo[2.2.2]octane, R3=H]

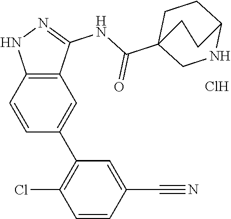 [SMILES: N#Cc1ccc(Cl)c(-c2ccc3[nH]nc(NC(=O)C45CCC(CC4)NC5)c3c2)c1 ; Cl]

Obtained 0.053 g (40% of yield). ¹H NMR (500 MHz, DMSO-d₆) δ ppm 1.70-2.18 (m, 10H), 3.12-3.17 (m, 1H), 3.27-3.29 (m, 1H), 7.42 (dd, J=8.69, 1.68 Hz, 1H), 7.55 (d, J=8.69 Hz, 1H), 7.68 (s, 1H), 7.81 (d, J=8.39 Hz, 1H), 7.88 (dd, J=8.24, 1.98 Hz, 1H), 7.92 (d, J=1.98 Hz, 1H), 8.74-8.93 (m, 2H), 10.19 (s, 1H), 12.99 (br. s., 1H). HRMS (ESI) calcd for C22H21ClN5O [M+H]⁺ 406.1429, found 406.1411.

### N-[5-(2-Chloro-5-cyanophenyl)-1H-indazol-3-yl]-1-azabicyclo[2.2.2]octane-3-carboxamide hydrochloride (cpd 188) [(I), R1=2-chloro-5-cyanophenyl, R2=1-azabicyclo[2.2.2]octane, R3=H]

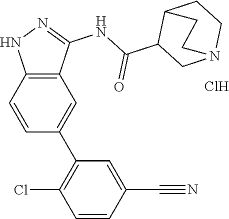 [SMILES: N#Cc1ccc(Cl)c(-c2ccc3[nH]nc(NC(=O)C4CN5CCC4CC5)c3c2)c1 ; Cl]

Obtained 0.027 g (56% of yield). ¹H NMR (500 MHz, DMSO-d₆) δ ppm 1.76-1.94 (m, 4H), 3.14-3.26 (m, 6H), 3.61-3.64 (m, 2H), 7.43 (dd, J=8.62, 1.60 Hz, 1H), 7.56 (d, J=8.69 Hz, 1H), 7.81 (d, J=8.39 Hz, 1H), 7.87-7.90 (m, 2H), 7.93 (d, J=1.98 Hz, 1H), 9.71 (br. s., 1H), 10.81 (s, 1H), 12.94 (br. s., 1H). HRMS (ESI) calcd for C22H21ClN5O [M+H]⁺ 406.1429, found 406.1427.

### 1-[(2,2-Dimethylpentanoyl)oxy]ethyl (3R)-3-{[5-(2-chloro-5-cyanophenyl)-1H-indazol-3-yl]carbamoyl}-piperidine-1-carboxylate (cpd 189) [(I), R1=2-chloro-5-cyanophenyl, R2=1-[(2,2-dimethylpentanoyl)oxy]ethyl piperidine-1-carboxylate, R3=H]

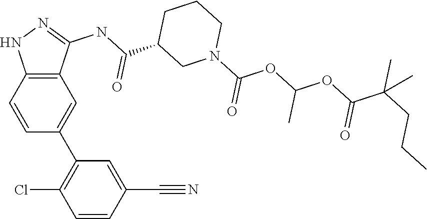 [SMILES: CCCC(C)(C)C(=O)OC(C)OC(=O)N1CCC[C@@H](C(=O)Nc2n[nH]c3ccc(-c4cc(C#N)ccc4Cl)cc23)C1]

Flash column chromatography (DCM/MeOH 95:5). Obtained 0.074 g (75% of yield) as white solid. 1H NMR (500 MHz, DMSO-d₆) δ ppm 0.73-0.86 (m, 3H), 1.02-1.19 (m, 8H), 1.36-143 (m, 6H), 1.63-1.77 (m, 2H), 2.00-2.09 (m, 1H), 2.60 (m, 1H), 2.82-3.02 (m, 2H), 3.82-3.98 (m, 2H), 6.61-6.65 (m, 1H), 7.42 (dd, J=8.69, 1.22 Hz, 1H), 7.54 (d, J=8.54 Hz, 1H), 7.82-7.86 (m, 2H), 7.87 (d, J=9.91 Hz, 1H), 7.92 (s, 1H), 10.58 (b. s., 1H), 12.87 (s, 1H). HRMS (ESI) calcd for C30H35ClN5O5 [M+H]⁺ 580.2321, found 580.2319.

### 1-[(2-Methylpropanoyl)oxy]ethyl (3R)-3-{[5-(2-chloro-5-cyanophenyl)-1H-indazol-3-yl]carbamoyl}piperidine-1-carboxylate (cpd 190) [(I), R1=2-chloro-5-cyanophenyl, R2=1-[(2-methylpropanoyl)oxy]ethyl piperidine-1-carboxylate, R3=H]

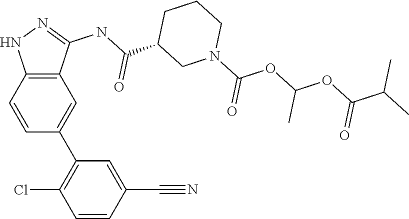 [SMILES: CC(OC(=O)C(C)C)OC(=O)N1CCC[C@@H](C(=O)Nc2n[nH]c3ccc(-c4cc(C#N)ccc4Cl)cc23)C1]

Flash column chromatography (DCM/MeOH 97:3). Obtained 0.070 g (41% of yield). 1H NMR (500 MHz, DMSO-d₆) δ ppm 0.92-1.14 (m, 6H), 1.36-1.44 (m, 4H), 1.63-1.75 (m, 2H), 1.99-2.04 (m, 1H), 2.60 (m, 1H), 2.86-3.04 (m, 2H), 3.78-4.14 (m, 2H), 6.65 (q, J=5.39 Hz, 1H), 7.42 (dd, J=8.69, 1.07 Hz, 1H), 7.54 (d, J=8.69 Hz, 1H), 7.80 (d, J=8.39 Hz, 1H), 7.84-7.89 (m, 2H), 7.93 (s, 1H), 10.56 (d, J=9.30 Hz, 1H), 12.87 (s, 1H). HRMS (ESI) calcd for C27H29ClN5O5Na [M+H+Na]⁺560.1671, found 560.1649.

### (Acetyloxy)methyl (3R)-3-{[5-(2-chloro-5-cyanophenyl)-1H-indazol-3-yl]carbamoyl}piperidine-1-carboxylate (cpd 191) [(I), R1=2-chloro-5-cyanophenyl, R2=(acetyloxy)methyl piperidine-1-carboxylate, R3=H]

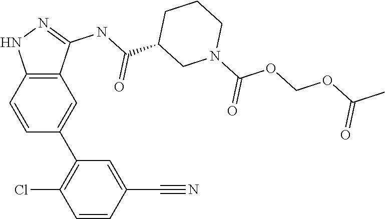 [SMILES: CC(=O)OCOC(=O)N1CCC[C@@H](C(=O)Nc2n[nH]c3ccc(-c4cc(C#N)ccc4Cl)cc23)C1]

Flash column chromatography (Hexane/EtOAc 70:30). Obtained 0.042 g (63% of yield). 1H NMR (500 MHz, DMSO-d₆) δ ppm 1.36-1.49 (m, 1H), 1.64-1.76 (m, 2H), 2.01-2.06 (m, 4H), 2.63 (m, 1H), 2.85-3.11 (m, 2H), 3.85-4.09 (m, 2H), 5.66 (m, 2H), 7.42 (dd, J=8.69, 1.37 Hz, 1H), 7.54 (d, J=8.69 Hz, 1H), 7.80-7.83 (m, 1H), 7.84-7.86 (m, 1H), 7.88 (m, 1H), 7.92-7.94 (m, 1H), 10.57 (s, 1H), 12.87 (s, 1H). HRMS (ESI) calcd for C24H23ClN5O5 [M+H]⁺ 496.9150, found 496.9132.

### [(2-Methylpropanoyl)oxy]methyl (3R)-3-{[5-(2-chloro-5-cyanophenyl)-1H-indazol-3-yl]carbamoyl}piperidine-1-carboxylate (cpd 192) [(I), R1=2-chloro-5-cyanophenyl, R2=[(2-methylpropanoyl)oxy]methyl piperidine-1-carboxylate, R3=H]

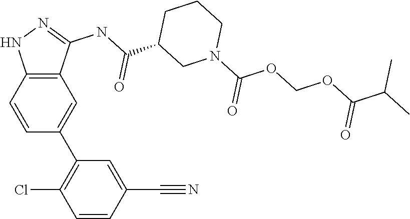 [SMILES: CC(C)C(=O)OCOC(=O)N1CCC[C@@H](C(=O)Nc2n[nH]c3ccc(-c4cc(C#N)ccc4Cl)cc23)C1]

Flash column chromatography (Hexane/EtOAc 70:30). Obtained 0.034 g (68% of yield). ¹H NMR (500 MHz, DMSO-d₆) δ ppm 1.09 (m, 6H), 1.34-1.40 (m, 1H), 1.64-1.77 (m, 2H), 2.03 (m, 1H), 2.61 (m, 1H), 2.84-3.11 (m, 2H), 3.84-4.12 (m, 2H), 5.61-5.74 (m, 2H), 7.43 (dd, J=8.69, 1.37 Hz, 1H), 7.55 (d, J=8.54 Hz, 1H), 7.82 (d, J=8.39 Hz, 1H), 7.85 (s, 1H), 7.88 (dd, J=8.24, 1.98 Hz, 1H), 7.94 (d, J=1.98 Hz, 1H), 10.58 (br. s., 1H), 12.88 (s, 1H).). HRMS (ESI) calcd for C26H27ClN5O5 [M+H]⁺ 524.9681, found 524.9702.

### [(2,2-Dimethylpropanoyl)oxy]methyl (3R)-3-{[5-(2-chloro-5-cyanophenyl)-1H-indazol-3-yl]carbamoyl}-piperidine-1-carboxylate (cpd 193) [(I), R1=2-chloro-5-cyanophenyl, R2=[(2,2-dimethylpropanoyl)oxy]methyl piperidine-1-carboxylate, R3=H]

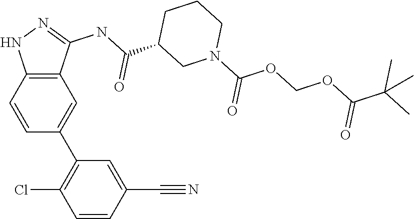 [SMILES: CC(C)(C)C(=O)OCOC(=O)N1CCC[C@@H](C(=O)Nc2n[nH]c3ccc(-c4cc(C#N)ccc4Cl)cc23)C1]

Flash column chromatography (Hexane/EtOAc 70:30). Obtained 0.040 g (63% of yield). ¹H NMR (500 MHz, DMSO-d₆) δ ppm 1.14 (s, 9H), 1.27-1.45 (m, 1H), 1.63-1.76 (m, 2H), 2.01-2.07 (m, 1H), 2.59 (m, 1H), 2.84-3.10 (m, 2H), 3.83-4.10 (m, 2H), 5.60-5.74 (m, 2H), 7.42 (dd, J=8.62, 1.30 Hz, 1H), 7.54 (d, J=8.69 Hz, 1H), 7.80 (d, J=8.39 Hz, 1H), 7.85 (s, 1H), 7.87 (dd, J=8.24, 1.98 Hz, 1H), 7.93 (d, J=1.98 Hz, 1H), 10.57 (br. s., 1H), 12.87 (s, 1H). HRMS (ESI) calcd for C27H29ClN5O5 [M+H]⁺ 538.9947, found 538.9933.

### 1-(Acetyloxy)ethyl (3R)-3-{[5-(2-chloro-5-cyanophenyl)-1H-indazol-3-yl]carbamoyl}piperidine-1-carboxylate (cpd 194) [(I), R1=2-chloro-5-cyanophenyl, R2=1-(acetyloxy)ethyl piperidine-1-carboxylate, R3=H]

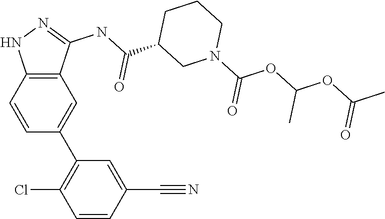 [SMILES: CC(=O)OC(C)OC(=O)N1CCC[C@@H](C(=O)Nc2n[nH]c3ccc(-c4cc(C#N)ccc4Cl)cc23)C1]

Flash column chromatography (Hexane/EtOAc 70:30). Obtained 0.047 g (62% of yield). ¹H NMR (500 MHz, DMSO-d₆) δ ppm 1.36-1.43 (m, 4H), 1.64-1.75 (m, 2H), 1.97-2.03 (m, 4H), 2.61 (m, 1H), 3.82-4.08 (m, 2H), 6.64 (br. s., 1H), 7.42 (dd, J=8.69, 1.07 Hz, 1H), 7.54 (d, J=8.69 Hz, 1H), 7.80 (d, J=8.24 Hz, 1H), 7.85 (s, 1H), 7.87 (dd, J=8.39, 1.98 Hz, 1H), 7.93 (d, J=1.83 Hz, 1H), 10.57 (s, 1H), 12.87 (s, 1H). HRMS (ESI) calcd for C25H25ClN5O5 [M+H]⁺ 510.1539, found 510.1532.

### 1-[(2,2-Dimethylpropanoyl)oxy]ethyl (3R)-3-{[5-(2-chloro-5-cyanophenyl)-1H-indazol-3-yl]carbamoyl}-piperidine-1-carboxylate (cpd 195) [(I), R1=2-chloro-5-cyanophenyl, R2=1-[(2,2-dimethylpropanoyl)oxy]ethyl piperidine-1-carboxylate, R3=H]

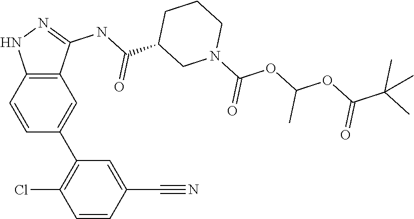 [SMILES: CC(OC(=O)N1CCC[C@@H](C(=O)Nc2n[nH]c3ccc(-c4cc(C#N)ccc4Cl)cc23)C1)OC(=O)C(C)(C)C]

Flash column chromatography (Hexane/EtOAc 70:30). Obtained 0.031 g (56% of yield). ¹H NMR (500 MHz, DMSO-d₆) δ ppm 1.07-1.15 (m, 9H), 1.37-1.43 (m, 4H), 1.64-1.75 (m, 2H), 2.00-2.06 (m, 1H), 2.59 (m, 1H), 2.85-3.13 (m, 2H), 3.83-4.04 (m, 2H), 6.63 (m, 1H), 7.41 (dd, J=8.69, 1.22 Hz, 1H), 7.54 (d, J=8.54 Hz, 1H), 7.80 (d, J=8.24 Hz, 1H), 7.84 (s, 1H), 7.87 (dd, J=8.24, 1.53 Hz, 1H), 7.92 (s, 1H), 10.45-10.74 (m, 1H), 12.87 (s, 1H). HRMS (ESI) calcd for C28H31ClN5O5 [M+H]⁺ 552.2008, found 552.1982.

### 2-Methyl-1-[(2-methylpropanoyl)oxy]propyl (3R)-3-{[5-(2-chloro-5-cyanophenyl)-1H-indazol-3-yl]carbamoyl}-piperidine-1-carboxylate (cpd 196) [(I), R1=2-chloro-5-cyanophenyl, R2=2-methyl-1-[(2-methylpropanoyl)oxy]propyl piperidine-1-carboxylate, R3=H]

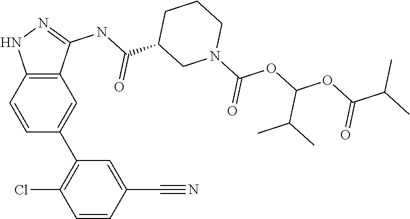 [SMILES: CC(C)C(=O)OC(OC(=O)N1CCC[C@@H](C(=O)Nc2n[nH]c3ccc(-c4cc(C#N)ccc4Cl)cc23)C1)C(C)C]

Flash column chromatography (Hexane/EtOAc 60:40). Obtained 0.054 g (51% of yield). ¹H NMR (500 MHz, DMSO-d₆) δ ppm 0.85-1.10 (m, 12H), 1.35-1.42 (m, 1H), 1.65-1.77 (m, 2H), 1.91-2.04 (m, 2H), 2.62 (m, 1H), 2.81-3.02 (m, 1H), 3.82-4.06 (m, 2H), 6.44 (d, J=4.58 Hz, 1H), 7.42 (d, J=8.85 Hz, 1H), 7.54 (d, J=8.69 Hz, 1H), 7.80 (d, J=8.39 Hz, 1H), 7.84 (s, 1H), 7.87 (dd, J=8.39, 1.83 Hz, 1H), 7.92 (s, 1H), 10.56 (d, J=19.37 Hz, 1H), 12.86 (s, 1H). HRMS (ESI) calcd for C29H31ClN5O5 [M+H]⁺ 566.2165, found 566.2159.

### 1-[(2,2-Dimethylpropanoyl)oxy]-2-methylpropyl (3R)-3-{[5-(2-chloro-5-cyanophenyl)-1H-indazol-3-yl]carbamoyl}piperidine-1-carboxylate (cpd 197) [(I), R1=2-chloro-5-cyanophenyl, R2=1-[(2,2-dimethylpropanoyl)oxy]-2-methylpropyl piperidine-1-carboxylate, R3=H]

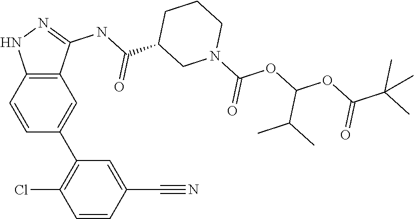 [SMILES: CC(C)C(OC(=O)N1CCC[C@@H](C(=O)Nc2n[nH]c3ccc(-c4cc(C#N)ccc4Cl)cc23)C1)OC(=O)C(C)(C)C]

Flash column chromatography (Hexane/EtOAc 50:50). Obtained 0.017 g (39% of yield). ¹H NMR (500 MHz, DMSO-d₆) δ ppm 0.85-0.91 (m, 6H), 1.07-1.17 (m, 9H), 1.34-1.42 (m, 1H), 1.64-1.77 (m, 2H), 1.95-2.08 (m, 2H), 2.60 (m, 1H), 2.89-3.05 (m, 1H), 3.86-4.09 (m, 2H), 6.41 (d, J=4.88 Hz, 1H), 7.42 (d, J=8.69 Hz, 1H), 7.54 (d, J=8.69 Hz, 1H), 7.80 (d, J=8.39 Hz, 1H), 7.84 (s, 1H), 7.87 (d, J=8.24 Hz, 1H), 7.92 (br. s., 1H), 10.42-10.67 (m, 1H), 12.86 (s, 1H). HRMS (ESI) calcd for C30H35ClN5O5 [M+H]⁺ 580.2321, found 580.2306.

### 1-{[(1-Methylcyclohexyl)carbonyl]oxy}ethyl (3R)-3-{[5-(2-chloro-5-cyanophenyl)-1H-indazol-3-yl]carbamoyl}-piperidine-1-carboxylate (cpd 198) [(I), R1=2-chloro-5-cyanophenyl, R2=1-{[(1-methylcyclohexyl)carbonyl]oxy}ethyl piperidine-1-carboxylate, R3=H]

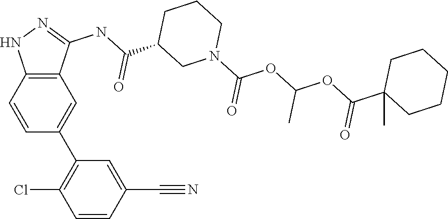 [SMILES: CC(OC(=O)N1CCC[C@@H](C(=O)Nc2n[nH]c3ccc(-c4cc(C#N)ccc4Cl)cc23)C1)OC(=O)C1(C)CCCCC1]

Flash column chromatography (DCM/MeOH 95:5). Obtained 0.063 g (83% of yield). ¹H NMR (500 MHz, DMSO-d₆) δ ppm 1.06-2.10 (m, 20H), 2.60 (m, 1H), 2.84-3.01 (m, 2H), 3.85-4.09 (m, 2H), 6.66 (m, 1H), 7.42 (dd, J=8.62, 1.30 Hz, 1H), 7.54 (d, J=8.69 Hz, 1H), 7.80-7.85 (m, 2H), 7.87 (d, J=8.69 Hz, 1H), 7.92 (br. s., 1H), 10.38-10.68 (m, 1H), 12.86 (s, 1H). HRMS (ESI) calcd for C31H35ClN5O5 [M+H]⁺ 592.2321, found 592.2307.

### 1-[(Tetrahydro-2H-pyran-4-ylcarbonyl)oxy]ethyl (3R)-3-{[5-(2-chloro-5-cyanophenyl)-1H-indazol-3-yl]carbamoyl}piperidine-1-carboxylate (cpd 199) [(I), R1=2-chloro-5-cyanophenyl, R2=1-[(tetrahydro-2H-pyran-4-ylcarbonyl)oxy]ethyl piperidine-1-carboxylate, R3=H]

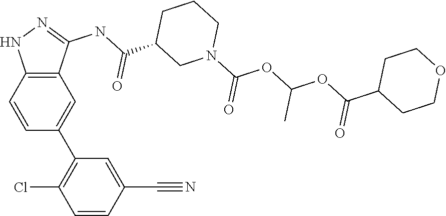 [SMILES: CC(OC(=O)C1CCOCC1)OC(=O)N1CCC[C@@H](C(=O)Nc2n[nH]c3ccc(-c4cc(C#N)ccc4Cl)cc23)C1]

Flash column chromatography (DCM/MeOH 95:5). Obtained 0.044 g (85% of yield). 1H NMR (500 MHz, DMSO-d₆) δ ppm 1.37-1.77 (m, 9H), 2.00-2.06 (m, 1H), 2.57-2.64 (m, 2H), 2.84-3.07 (m, 1H), 3.29-3.32 (m, 2H), 3.75-4.08 (m, 4H), 6.67 (m, 1H), 7.42 (d, J=8.85 Hz, 1H), 7.54 (d, J=8.54 Hz, 1H), 7.80 (d, J=8.24 Hz, 1H), 7.85 (s, 1H), 7.87 (dd, J=8.24, 1.83 Hz, 1H), 7.93 (s, 1H), 10.48-10.66 (m, 1H), 12.87 (s, 1H). HRMS (ESI) calcd for C29H31ClN5O6 [M+H]⁺ 581.0314, found 581.0355.

### 1-[(2,2-Dimethylpropanoyl)oxy]propyl (3R)-3-{[5-(2-chloro-5-cyanophenyl)-1H-indazol-3-yl]carbamoyl}-piperidine-1-carboxylate (cpd 200) [(I), R1=2-chloro-5-cyanophenyl, R2=1-[(2,2-dimethylpropanoyl)oxy]propyl piperidine-1-carboxylate, R3=H]

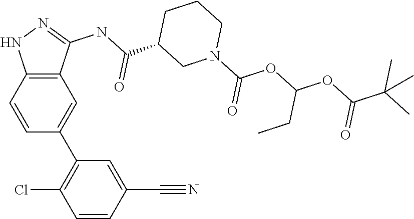 [SMILES: CCC(OC(=O)N1CCC[C@@H](C(=O)Nc2n[nH]c3ccc(-c4cc(C#N)ccc4Cl)cc23)C1)OC(=O)C(C)(C)C]

Flash column chromatography (DCM/MeOH 97:3). Obtained 0.037 g (79% of yield). 1H NMR (500 MHz, DMSO-d₆) δ ppm 0.81-0.89 (m, 3H), 1.07-1.15 (m, 9H), 1.33-1.42 (m, 1H), 1.65-1.79 (m, 4H), 1.99-2.09 (m, 1H), 2.60 (m, 1H), 2.85-3.19 (m, 2H), 3.80-4.08 (m, 2H), 6.53 (t, J=5.19 Hz, 1H), 7.42 (d, J=9.30 Hz, 1H), 7.54 (d, J=8.54 Hz, 1H), 7.80-7.85 (m, 2H), 7.87 (dd, J=8.39, 1.22 Hz, 1H), 7.92 (s, 1H), 10.34-10.71 (m, 1H), 12.86 (s, 1H). HRMS (ESI) calcd for C29H33ClN5O5 [M+H]⁺ 567.0479, found 567.0448.

### N-[5-(2,6-Dichlorophenyl)-1H-indazol-3-yl]-4-hydroxycyclohexanecarboxamide (cpd 201) [(I), R1=2,6-dichlorophenyl, R2=4-hydroxycyclohexane, R3=H]

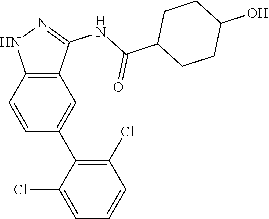 [SMILES: O=C(Nc1n[nH]c2ccc(-c3c(Cl)cccc3Cl)cc12)C1CCC(O)CC1]

To a solution of 4-(benzyloxy)-N-[5-(2,6-dichlorophenyl)-1-trityl-1H-indazol-3-yl]cyclohexanecarboxamide (0.02 g, 0.027 mmol) in MeOH (3 mL), 10% Pd/C (0.002 g, 20% w/w) and 1,4-cyclohexadiene (0.025 mL, 0.27 mmol) were added. The mixture was heated in a CEM explorer microwave™ at 120° C. for 15 min. After filtration and evaporation to dryness the crude was grinded with diethyl ether. Obtained 0.008 g (73% of yield). ¹H NMR (500 MHz, DMSO-d₆) δ ppm 1.10-1.24 (m, 2H), 1.39-1.68 (m, 4H), 1.80-1.92 (m, 2H), 2.33-2.39 (m, 1H), 3.74-3.78 (m, 1H), 4.55 (br. s., 1H), 7.16-7.19 (m, 1H), 7.44 (t, J=7.93 Hz, 1H), 7.52 (d, J=9.30 Hz, 1H), 7.58 (d, J=9.00 Hz, 2H), 7.77 (s, 1H), 10.31 (br. s., 1H), 12.76 (br. s., 1H). HRMS (ESI) calcd for C20H2OC12N3O2 [M+H]⁺ 404.0927, found 404.0930.

### Example 2 (Chiral Resolution)

### (+)-N-[5-(2-Chloro-5-cyanophenyl)-1H-indazol-3-yl]-6,6-dimethylpiperidine-3-carboxamide (cpd 202) [(I), R1=2-chloro-5-cyanophenyl, R2=2,2-dimethylpiperidine, R3=H]

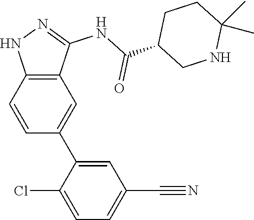 [SMILES: CC1(C)CC[C@@H](C(=O)Nc2n[nH]c3ccc(-c4cc(C#N)ccc4Cl)cc23)CN1]

Obtained by chiral resolution of (±)-N-[5-(2-chloro-5-cyanophenyl)-1H-indazol-3-yl]-6,6-dimethylpiperidine-3-carboxamide. Absolute stereochemistry was not determined.

Sample preparation: Racemic material was dissolved in ethanol to 5 mg/mL and filtrated through membrane with pore sized 0.45 μm.

### Analytical Separation Method:

Instrument: Shimadzu LC-20AB analytical HPLC system; Column: Daicel Chiralpak AD-3, 150×4.6 mm i.d., 3 μm; Mobile Phase: Mobile phase A for n-hexane and Mobile phase B for EtOH (0.05% IPAm, v/v); Gradient A:B=70:30 isocratic elution mode; Flow rate: 1.0 ml/min; Column temperature: 30° C.

### Preparative Separation Method

Instrument: Waters SFC80Q preparative SFC; Column: Daicel Chiralpak IG, 250×30 mm I.D. 10 μm; Mobile phase: A for CO₂ and B for EtOH (0.1% NH₃H₂O, v/v); Gradient: B %=60% isocratic elution mode; Flow rate: 80 g/min; Wavelength: 220 nm.

Faster eluting enantiomer: time 6.062 min., recovery 70%, ee 100%, HPLC purity 97.6%. ¹H NMR (500 MHz, DMSO-d₆) δ ppm 1.03-1.06 (m, 6H), 1.16-1.34 (m, 2H), 1.42-1.58 (m, 1H), 1.70-1.91 (m, 2H), 2.80-3.02 (m, 2H), 7.40 (dd, J=8.69, 1.37 Hz, 1H), 7.52 (d, J=8.69 Hz, 1H), 7.80 (d, J=8.24 Hz, 1H), 7.86 (s, 1H), 7.87 (dd, J=8.24, 1.98 Hz, 1H), 7.92 (d, J=2.14 Hz, 1H), 10.57 (s, 1H), 12.82 (br. s., 1H). HRMS (ESI) calcd for C22H23ClN5O [M+H]⁺ 408.1586, found 408.1587.

According to this same methodology, starting from the opportune racemates, the following chiral compounds were obtained:

### (−)-N-[5-(2-Chloro-5-cyanophenyl)-1H-indazol-3-yl]-6,6-dimethylpiperidine-3-carboxamide (cpd 203) [(I), R1=2-chloro-5-cyanophenyl, R2=2,2-dimethylpiperidine, R3=H]

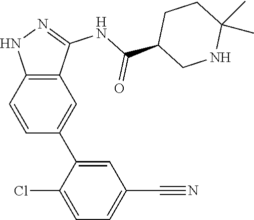 [SMILES: CC1(C)CC[C@H](C(=O)Nc2n[nH]c3ccc(-c4cc(C#N)ccc4Cl)cc23)CN1]

Obtained by chiral resolution of (±)-N-[5-(2-chloro-5-cyanophenyl)-1H-indazol-3-yl]-6,6-dimethylpiperidine-3-carboxamide. Absolute stereochemistry was not determined. Slower eluting enantiomer: time 9.604 min., recovery 73%, ee 99.7%, HPLC purity 98.1%. ¹H NMR (500 MHz, DMSO-d₆) δ ppm 1.03-1.06 (m, 6H), 1.16-1.34 (m, 2H), 1.42-1.58 (m, 1H), 1.70-1.91 (m, 2H), 2.80-3.02 (m, 2H), 7.40 (dd, J=8.69, 1.37 Hz, 1H), 7.52 (d, J=8.69 Hz, 1H), 7.80 (d, J=8.24 Hz, 1H), 7.86 (s, 1H), 7.87 (dd, J=8.24, 1.98 Hz, 1H), 7.92 (d, J=2.14 Hz, 1H), 10.57 (s, 1H), 12.82 (br. s., 1H).). HRMS (ESI) calcd for C22H23ClN5O [M+H]⁺ 408.1586, found 408.1586.

### (8+)-N-[5-(2-Chloro-5-cyanophenyl)-1H-indazol-3-yl]-6-azaspiro[4.5]decane-8-carboxamide hydrochloride (cpd 204) [(I), R1=2-chloro-5-cyanophenyl, R2=6-azaspiro[4.5]decane, R3=H]

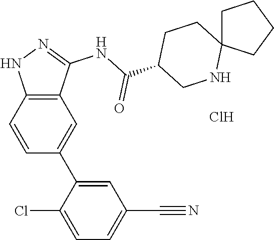 [SMILES: N#Cc1ccc(Cl)c(-c2ccc3[nH]nc(NC(=O)[C@@H]4CCC5(CCCC5)NC4)c3c2)c1 ; Cl]

Obtained by chiral resolution of (8±)-N-[5-(2-chloro-5-cyanophenyl)-1H-indazol-3-yl]-6-azaspiro[4.5]decane-8-carboxamide. Absolute stereochemistry was not determined. Faster eluting enantiomer: time 9.442 min., recovery 70%, ee 99.6%, HPLC purity 98.4%. Converted in the hydrochloride salt by treatment of the solution in DCM with 4 M HCl in dioxane (2 eq.). ¹H NMR (500 MHz, DMSO-d₆) δ ppm 1.56-1.87 (m, 12H), 2.05-2.10 (m, 1H), 2.95 (m, 1H), 3.07-3.09 (m, 1H), 3.33-3.35 (m, 1H), 7.41 (dd, J=8.69, 1.52 Hz, 1H), 7.54 (d, J=8.69 Hz, 1H), 7.81 (d, J=8.24 Hz, 1H), 7.87 (s, 1H), 7.88 (dd, J=8.39, 2.13 Hz, 1H), 7.92 (d, J=1.83 Hz, 1H), 8.79 (d, J=10.07 Hz, 1H), 9.05 (br. s., 1H), 10.76 (s, 1H), 12.93 (br. s., 1H). HRMS (ESI) calcd for C24H25ClN5O [M+H]⁺ 434.1742, found 434.1729.

### (8-)-N-[5-(2-Chloro-5-cyanophenyl)-1H-indazol-3-yl]-6-azaspiro[4.5]decane-8-carboxamide hydrochloride (cpd 205) [(I), R1=2-chloro-5-cyanophenyl, R2=6-azaspiro[4.5]decane, R3=H]

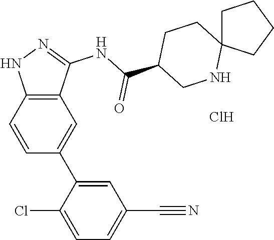 [SMILES: N#Cc1ccc(Cl)c(-c2ccc3[nH]nc(NC(=O)[C@H]4CCC5(CCCC5)NC4)c3c2)c1 ; Cl]

Obtained by chiral resolution of (8±)-N-[5-(2-chloro-5-cyanophenyl)-1H-indazol-3-yl]-6-azaspiro[4.5]decane-8-carboxamide. Absolute stereochemistry was not determined. Slower eluting enantiomer: time 11.505 min., recovery 64%, ee 99.6%, HPLC purity 97.6%. Converted in the hydrochloride salt by treatment of the solution in DCM with 4 M HCl in dioxane (2 eq.). ¹H NMR (500 MHz, DMSO-d₆) δ ppm 1.56-1.87 (m, 12H), 2.05-2.10 (m, 1H), 2.95 (m, 1H), 3.07-3.09 (m, 1H), 3.33-3.35 (m, 1H), 7.41 (dd, J=8.69, 1.52 Hz, 1H), 7.54 (d, J=8.69 Hz, 1H), 7.81 (d, J=8.24 Hz, 1H), 7.87 (s, 1H), 7.88 (dd, J=8.39, 2.13 Hz, 1H), 7.92 (d, J=1.83 Hz, 1H), 8.79 (d, J=10.07 Hz, 1H), 9.05 (br. s., 1H), 10.76 (s, 1H), 12.93 (br. s., 1H). HRMS (ESI) calcd for C24H25ClN5O [M+H]⁺ 434.1742, found 434.1731.

### (3+)-N-[5-(2-Chloro-5-cyanophenyl)-1H-indazol-3-yl]-1-azaspiro[5.5]undecane-3-carboxamide (cpd 206) [(I), R1=2-chloro-5-cyanophenyl, R2=1-azaspiro[5.5]undecane, R3=H]

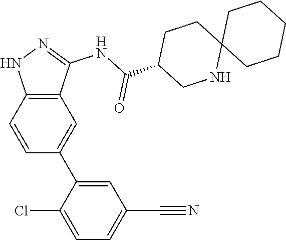 [SMILES: N#Cc1ccc(Cl)c(-c2ccc3[nH]nc(NC(=O)[C@@H]4CCC5(CCCCC5)NC4)c3c2)c1]

Obtained by chiral resolution of (3±)-N-[5-(2-chloro-5-cyanophenyl)-1H-indazol-3-yl]-1-azaspiro[5.5]undecane-3-carboxamide. Absolute stereochemistry was not determined. Faster eluting enantiomer: time 10.736 min., recovery 72%, ee 99.2%, HPLC purity 98.9%. ¹H NMR (500 MHz, DMSO-d₆) δ ppm 1.07-1.44 (m, 8H), 1.45-1.70 (m, 4H), 1.76-1.81 (m, 2H), 2.83-2.92 (m, 2H), 3.32 (m, 1H), 7.40 (dd, J=8.85, 1.37 Hz, 1H), 7.52 (d, J=8.69 Hz, 1H), 7.80 (d, J=8.39 Hz, 1H), 7.86 (s, 1H), 7.87 (dd, J=8.39, 2.14 Hz, 1H), 7.92 (d, J=1.83 Hz, 1H), 10.59 (s, 1H), 12.82 (s, 1H). HRMS (ESI) calcd for C25H27ClN5O [M+H]⁺ 448.1899, found 448.1897.

### (3-)-N-[5-(2-Chloro-5-cyanophenyl)-1H-indazol-3-yl]-1-azaspiro[5.5]undecane-3-carboxamide (cpd 207) [(I), R1=2-chloro-5-cyanophenyl, R2=1-azaspiro[5.5]undecane, R3=H]

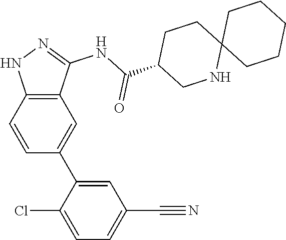 [SMILES: N#Cc1ccc(Cl)c(-c2ccc3[nH]nc(NC(=O)[C@@H]4CCC5(CCCCC5)NC4)c3c2)c1]

Obtained by chiral resolution of (3±)-N-[5-(2-chloro-5-cyanophenyl)-1H-indazol-3-yl]-1-azaspiro[5.5]undecane-3-carboxamide. Absolute stereochemistry was not determined. Slower eluting enantiomer: time 18.765 min., recovery 61%, ee 99.5%, HPLC purity 99.1%. 1H NMR (500 MHz, DMSO-d₆) δ ppm 1.07-1.44 (m, 8H), 1.45-1.70 (m, 4H), 1.76-1.81 (m, 2H), 2.83-2.92 (m, 2H), 3.32 (m, 1H), 7.40 (dd, J=8.85, 1.37 Hz, 1H), 7.52 (d, J=8.69 Hz, 1H), 7.80 (d, J=8.39 Hz, 1H), 7.86 (s, 1H), 7.87 (dd, J=8.39, 2.14 Hz, 1H), 7.92 (d, J=1.83 Hz, 1H), 10.59 (s, 1H), 12.82 (s, 1H). HRMS (ESI) calcd for C25H27ClN5O [M+H]⁺ 448.1899, found 448.1895.

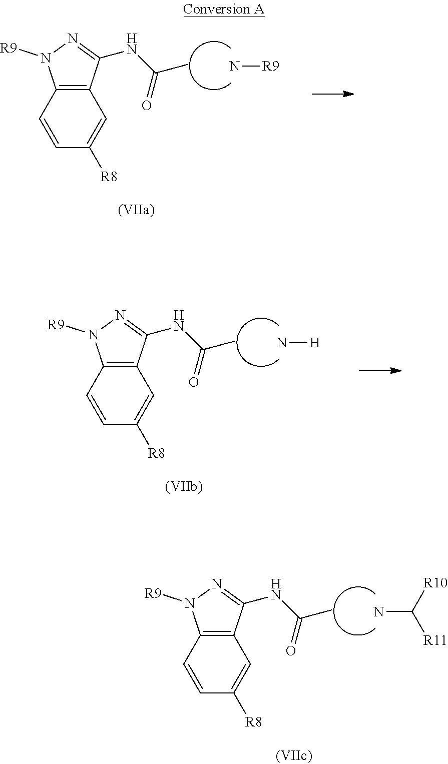

### N-(5-Bromo-1-trityl-1H-indazol-3-yl)piperidine-4-carboxamide [(VIIb), R1=Br, R2=piperidine, R9=1,1′,1″-methanetriyltribenzene]

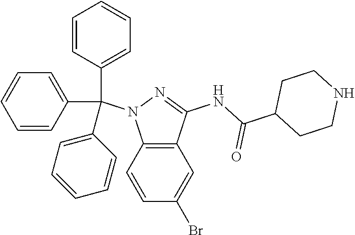 [SMILES: O=C(Nc1nn(C(c2ccccc2)(c2ccccc2)c2ccccc2)c2ccc(Br)cc12)C1CCNCC1]

To a partial solution of 9H-fluoren-9-ylmethyl 4-[(5-bromo-1-trityl-1H-indazol-3-yl)carbamoyl]piperidine-1-carboxylate (6.53 g, 8.30 mmol), in MeOH (200 mL), piperidine (10 mL) was added. The resulting mixture was stirred at room temperature for 24 h, then the solution was evaporated to dryness. The crude obtained was purified by flash column chromatography (DCM/MeOH/33% aqueous ammonia 90:10:1) to afford N-(5-bromo-1-trityl-1H-indazol-3-yl)piperidine-4-carboxamide (3.95 g, 84% of yield) as white solid. ¹H NMR (500 MHz, DMSO-d6) δ ppm 1.45-1.58 (m, 2H), 1.70 (d, J=11.74 Hz, 2H), 2.45 (m, 2H), 2.53-2.58 (m, 1H), 2.96 (d, J=12.20 Hz, 2H), 6.32 (d, J=9.15 Hz, 1H), 7.17-7.20 (m, 7H), 7.25-7.30 (m, 3H), 7.31-7.36 (m, 6H), 7.90 (s, 1H), 10.48 (s, 1H). HRMS (ESI) calcd for C32H30BrN4O [M+H]⁺ 587.1417, found 587.1415.

### N-(5-Bromo-1-trityl-1H-indazol-3-yl)-1-methylpiperidine-4-carboxamide [(VIIc), R1=Br, R2=1-methylpiperidine, R9=1,1′,1″-methanetriyltribenzene]

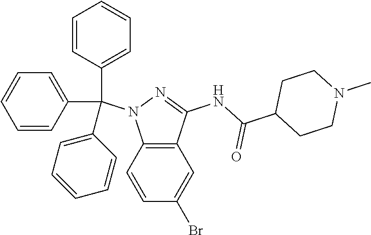 [SMILES: CN1CCC(C(=O)Nc2nn(C(c3ccccc3)(c3ccccc3)c3ccccc3)c3ccc(Br)cc23)CC1]

To a solution of N-(5-bromo-1-trityl-1H-indazol-3-yl)piperidine-4-carboxamide (3.95 g, 6.99 mmol) in DCM (150 mL), formaldehyde (37% water, 1.7 mL, 20.97 mmol), glacial acetic acid (5 drops) and sodium triacetoxyborohydride (4.44 g, 20.97 mmol) were added. The reaction was stirred at room temperature for 1 h, then washed with aqueous NaHCO₃ (50 mL), water (50 mL) and brine (50 mL). The organic phase was separated, dried with anhydrous Na₂SO₄, filtered and evaporated. The crude was purified by flash column chromatography (DCM/MeOH/33% aqueous ammonia 90:10:0.5) to give N-(5-bromo-1-trityl-1H-indazol-3-yl)-1-methylpiperidine-4-carboxamide (3.08 g, 76% of yield) as white solid. ¹H NMR (401 MHz, DMSO-d₆) δ ppm 1.62-1.87 (m, 6H), 2.19 (s, 3H), 2.52-2.54 (m, 1H), 2.76-2.94 (m, 2H), 6.32 (d, J=9.28 Hz, 1H), 7.16-7.21 (m, 6H), 7.27-7.36 (m, 9H), 7.93 (s, 1H), 10.45-10.55 (s, 1H). HRMS (ESI) calcd for C33H32BrN4O [M+H]⁺ 579.1754, found 579.1764. According to this same methodology, reacting N-(5-bromo-1-trityl-1H-indazol-3-yl)piperidine-4-carboxamide with the suitable reagents, the following compounds were prepared:

### N-(5-Bromo-1-trityl-1H-indazol-3-yl)-1-butylpiperidine-4-carboxamide [(VIIc), R1=Br, R2=1-butylpiperidine, R9=1,1′,1″-methanetriyltribenzene]

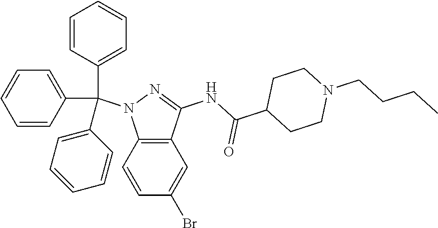 [SMILES: CCCCN1CCC(C(=O)Nc2nn(C(c3ccccc3)(c3ccccc3)c3ccccc3)c3ccc(Br)cc23)CC1]

Starting from N-(5-bromo-1-trityl-1H-indazol-3-yl)piperidine-4-carboxamide and butyraldehyde. Used as crude (0.11 g, 95% of yield). ¹H NMR (500 MHz, DMSO-d₆) δ ppm 0.87 (m, 3H), 1.23-1.44 (m, 4H), 1.78-2.00 (m, 6H), 2.20-2.28 (m, 2H), 2.53-2.56 (m, 1H), 2.87-2.89 (m, 2H), 6.32 (d, J=9.30 Hz, 1H), 7.15-7.21 (d, J=7.47 Hz, 7H), 7.26-7.30 (m, 3H), 7.30-7.36 (m, 6H), 7.91 (s, 1H), 10.52 (s, 1H). HRMS (ESI) calcd for C36H38BrN4O [M+H]⁺ 621.2224, found 621.2210.

### tert-Butyl (3-{4-[(5-bromo-1-trityl-1H-indazol-3-yl)carbamoyl]piperidin-1-yl}propyl)carbamate [(VIIc), R1=Br, R2=tert-butyl [3-(piperidin-1-yl)propyl]carbamate, R9=1,1′,1″-methanetriyltribenzene]

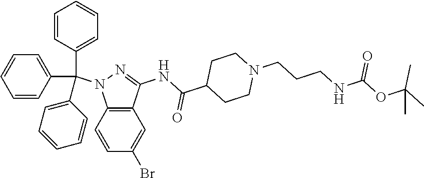 [SMILES: CC(C)(C)OC(=O)NCCCN1CCC(C(=O)Nc2nn(C(c3ccccc3)(c3ccccc3)c3ccccc3)c3ccc(Br)cc23)CC1]

Starting from N-(5-bromo-1-trityl-1H-indazol-3-yl)piperidine-4-carboxamide and tert-butyl (3-oxopropyl)carbamate. Flash column chromatography (DCM/MeOH 92:8). Obtained 0.11 g (72% of yield). ¹H NMR (500 MHz, DMSO-d₆) δ ppm 1.37 (s, 9H), 1.48-1.89 (m, 10H), 2.25 (m, 1H), 2.77-3.00 (m, 3H), 6.32 (d, J=9.15 Hz, 1H), 6.82 (br. s., 1H), 7.15-7.20 (m, 7H), 7.26-7.31 (m, 3H), 7.31-7.36 (m, 6H), 7.91 (s, 1H), 10.53 (br. s., 1H). HRMS (ESI) calcd for C40H45BrN5O3 [M+H]⁺ 722.2701, found 722.2684.

### N-(5-Bromo-1-trityl-1H-indazol-3-yl)-1-(1-methoxypropan-2-yl)piperidine-4-carboxamide carbamate [(VIIc), R1=Br, R2=1-(1-methoxypropan-2-yl)piperidine, R9=1,1′,1″-methanetriyltribenzene]

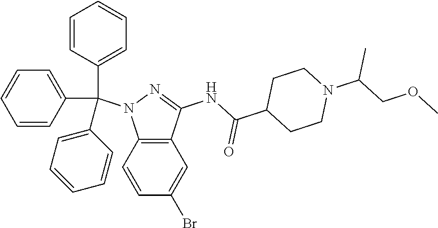 [SMILES: COCC(C)N1CCC(C(=O)Nc2nn(C(c3ccccc3)(c3ccccc3)c3ccccc3)c3ccc(Br)cc23)CC1]

Starting from N-(5-bromo-1-trityl-1H-indazol-3-yl)piperidine-4-carboxamide and 1-methoxypropan-2-one. Flash column chromatography (DCM/MeOH 90:10). Obtained 0.06 g (64% of yield). LCMS (ESI) m/z 638 (M+H)⁺.

### N-(5-Bromo-1-trityl-1H-indazol-3-yl)-1-(2-methylpropyl)piperidine-4-carboxamide [(VIIc), R1=Br, R2=1-(2-methylpropyl)piperidine, R9=1,1′,1″-methanetriyltribenzene]

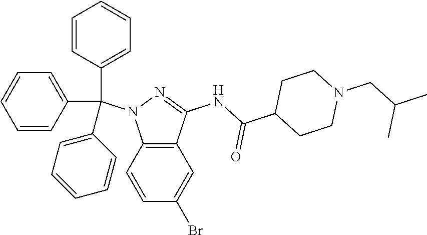 [SMILES: CC(C)CN1CCC(C(=O)Nc2nn(C(c3ccccc3)(c3ccccc3)c3ccccc3)c3ccc(Br)cc23)CC1]

Starting from N-(5-bromo-1-trityl-1H-indazol-3-yl)piperidine-4-carboxamide and 2-methylpropanal. Used as crude (0.14 g, 85% of yield). LCMS (ESI) m/z 622 (M+H)⁺.

### N-(5-Bromo-1-trityl-1H-indazol-3-yl)-1-(3-hydroxy-2,2-dimethylpropyl)piperidine-4-carboxamide [(VIIc), R1=Br, R2=1-(3-hydroxy-2,2-dimethylpropyl)piperidine, R9=1,1′,1″-methanetriyltribenzene]

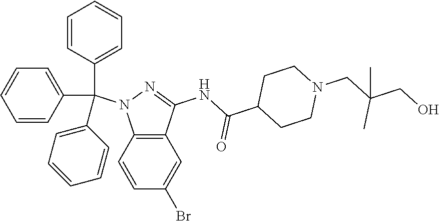 [SMILES: CC(C)(CO)CN1CCC(C(=O)Nc2nn(C(c3ccccc3)(c3ccccc3)c3ccccc3)c3ccc(Br)cc23)CC1]

Starting from N-(5-bromo-1-trityl-1H-indazol-3-yl)piperidine-4-carboxamide and 3-hydroxy-2,2-dimethylpropanal. Flash column chromatography (DCM/MeOH 93:7). Obtained 0.12 g (63% of yield). ¹H NMR (401 MHz, DMSO-d₆) δ ppm 0.78 (s, 6H), 1.55-1.81 (m, 4H), 1.98-2.24 (m, 4H), 2.40 (m, 1H), 2.81-2.86 (m, 2H), 3.16 (s, 2H), 4.56 (br. s., 1H), 6.32 (d, J=9.15 Hz, 1H), 7.18 (d, J=7.57 Hz, 7H), 7.24-7.39 (m, 9H), 7.91 (s, 1H), 10.48 (s, 1H). HRMS (ESI) calcd for C37H40BrN4O2 [M+H]⁺ 651.2329, found 651.2341.

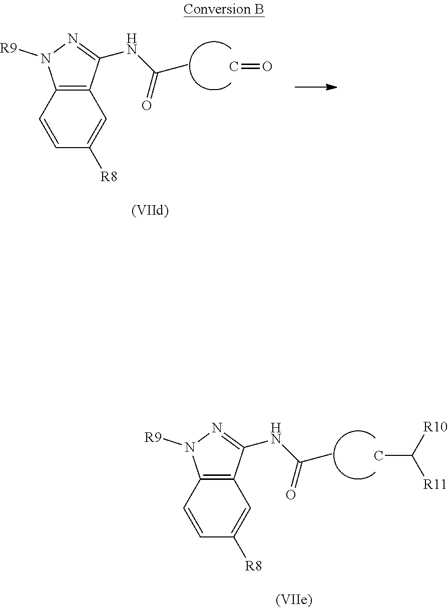

### N-(5-Bromo-1-trityl-1H-indazol-3-yl)-3-(methylamino)cyclobutanecarboxamide carboxamide [(Vile), R1=Br, R2=N-methylcyclobutanamine, R9=1,1′,1″-methanetriyltribenzene]

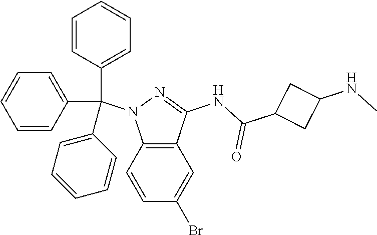 [SMILES: CNC1CC(C(=O)Nc2nn(C(c3ccccc3)(c3ccccc3)c3ccccc3)c3ccc(Br)cc23)C1]

To a solution of N-(5-bromo-1-trityl-1H-indazol-3-yl)-3-oxocyclobutanecarboxamide (0.06 g, 0.109 mmol) in DCM (5 mL), 2M methylamine in THE (0.55 mL, 1.09 mmol), glacial acetic acid (2 drops) and sodium triacetoxyborohydride (0.069 g, 0.327 mmol) were added. The reaction was stirred at room temperature for 2 h, then washed with aqueous NaHCO₃ (10 mL), water (10 mL) and brine (10 mL). The organic phase was separated, dried with anhydrous Na₂SO₄, filtered and evaporated. The crude was purified by flash column chromatography (DCM/MeOH/33% aqueous ammonia 95:5:0.5) to give N-(5-bromo-1-trityl-1H-indazol-3-yl)-3-(methylamino)cyclobutanecarboxamide carboxamide (0.048 g, 78% of yield) as white solid. ¹H NMR (500 MHz, DMSO-d₆) δ ppm 1.86-1.98 (m, 2H), 2.18 (s, 3H), 2.29-2.37 (m, 2H), 2.83-3.06 (m, 1H), 3.27 (m, 1H), 6.33 (d, J=9.15 Hz, 1H), 7.18 (d, J=7.47 Hz, 7H), 7.26-7.30 (m, 3H), 7.31-7.35 (m, 6H), 7.95 (d, J=14.95 Hz, 1H), 10.44-10.53 (m, 1H). HRMS (ESI) calcd for C32H30rN40 [M+H]⁺ 565.1598, found 565.1598.

According to this same methodology, but employing suitable intermediate, the following compound was prepared:

### N-(5-Bromo-1-trityl-1H-indazol-3-yl)-3-(dimethylamino)cyclobutanecarboxamide [(Vile), R1=Br, R2=N-dimethylcyclobutanamine, R9=1,1′,1″-methanetriyltribenzene]

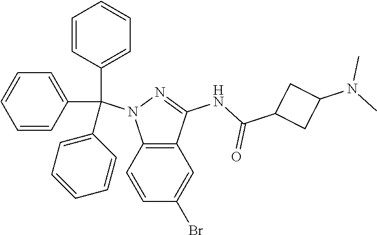 [SMILES: CN(C)C1CC(C(=O)Nc2nn(C(c3ccccc3)(c3ccccc3)c3ccccc3)c3ccc(Br)cc23)C1]

Starting from N-(5-bromo-1-trityl-1H-indazol-3-yl)-3-oxocyclobutanecarboxamide and 2M dimethylamine in THF. Flash column chromatography (DCM/MeOH/33% aqueous ammonia 95:5:0.5). Obtained 0.093 g (68% of yield). ¹H NMR (500 MHz, DMSO-d₆) δ ppm 1.93-2.07 (m, 8H), 2.16-2.26 (m, 2H), 2.54 (m, 1H), 2.92 (m, 1H), 6.33 (d, J=9.15 Hz, 1H), 7.18 (d, J=7.63 Hz, 7H), 7.26-7.30 (m, 3H), 7.31-7.35 (m, 6H), 7.95 (s, 1H), 10.52 (s, 1H). HRMS (ESI) calcd for C33H32BrN4O [M+H]⁺ 579.1754, found 579.1766.

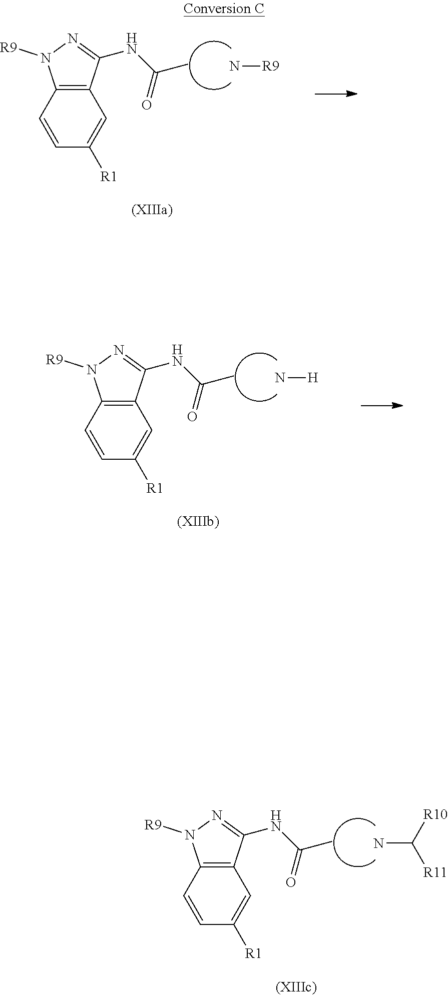

The compounds were prepared according to the same procedure reported for conversion A.

### N-[5-(2-Chloro-5-cyanophenyl)-1-trityl-1H-indazol-3-yl]piperidine-4-carboxamide [(XIIIb), R1=2-chloro-5-cyanophenyl, R2=piperidine, R9=1,1′,1″-methanetriyltribenzene]

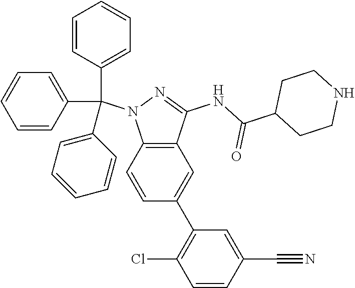 [SMILES: N#Cc1ccc(Cl)c(-c2ccc3c(c2)c(NC(=O)C2CCNCC2)nn3C(c2ccccc2)(c2ccccc2)c2ccccc2)c1]

Starting from 9H-fluoren-9-ylmethyl 4-{[5-(2-chloro-5-cyanophenyl)-1-trityl-1H-indazol-3-yl]carbamoyl}piperidine-1-carboxylate. Flash column chromatography (DCM/MeOH/33% aqueous ammonia 90:10:1). Obtained 0.59 g (95% of yield). ¹H NMR (500 MHz, DMSO-d₆) δ ppm 1.46-1.54 (m, 2H), 1.68-1.73 (m, 2H), 2.44 (t, J=11.74 Hz, 2H), 2.53-2.59 (m, 1H), 2.95 (d, J=12.20 Hz, 2H), 6.45 (d, J=9.00 Hz, 1H), 7.16 (d, J=8.69 Hz, 1H), 7.23-7.26 (m, 5H), 7.27-7.31 (m, 3H), 7.33-7.38 (m, 6H), 7.77 (d, J=8.24 Hz, 1H), 7.83-7.85 (m, 2H), 7.88 (s, 1H), 7.88 (d, J=1.53 Hz, 1H), 10.45 (s, 1H). HRMS (ESI) calcd for C39H33ClN5O [M+H]⁺ 622.2368, found 622.2368.

### 1-Butyl-N-[5-(2-chloro-5-cyanophenyl)-1-trityl-1H-indazol-3-yl]piperidine-4-carboxamide [(XIII c), R1=2-chloro-5-cyanophenyl, R2=1-butylpiperidine, R9=1,1′,1″-methanetriyltribenzene]

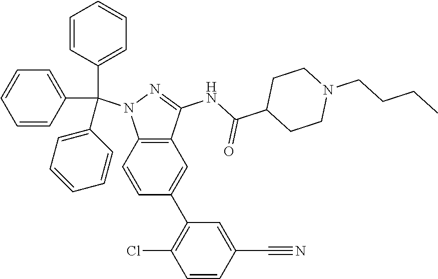 [SMILES: CCCCN1CCC(C(=O)Nc2nn(C(c3ccccc3)(c3ccccc3)c3ccccc3)c3ccc(-c4cc(C#N)ccc4Cl)cc23)CC1]

Starting from N-[5-(2-chloro-5-cyanophenyl)-1-trityl-1H-indazol-3-yl]piperidine-4-carboxamide and butyraldehyde. Flash column chromatography (DCM/MeOH 95:5). Obtained 0.063 g (83% of yield). ¹H NMR (500 MHz, DMSO-d₆) δ ppm 0.87 (t, J=7.32 Hz, 3H), 1.26 (dq, J=14.77, 7.23 Hz, 2H), 1.38 (quin, J=7.21 Hz, 2H), 1.53-1.70 (m, 2H), 1.76-1.89 (m, 4H), 2.22-2.24 (m, 2H), 2.43 (m, 1H), 2.86-2.88 (m, 2H), 6.46 (d, J=8.85 Hz, 1H), 7.16 (d, J=8.69 Hz, 1H), 7.23-7.25 (m, 6H), 7.27-7.32 (m, 3H), 7.32-7.38 (m, 6H), 7.77 (d, J=8.40 Hz, 1H), 7.83 (br. s., 1H), 7.85 (dd, J=8.20, 2.00 Hz, 1H), 7.88 (d, J=1.53 Hz, 1H), 10.48 (s, 1H). HRMS (ESI) calcd for C43H41ClN5O [M+H]⁺ 678.2994, found 678.3004.

### N-[5-(2-Chloro-5-cyanophenyl)-1-trityl-1H-indazol-3-yl]-1-(propan-2-yl)piperidine-4-carboxamide [(XIII c), R1=2-chloro-5-cyanophenyl, R2=1-(propan-2-yl)piperidine, R9=1,1′,1″-methanetriyltribenzene]

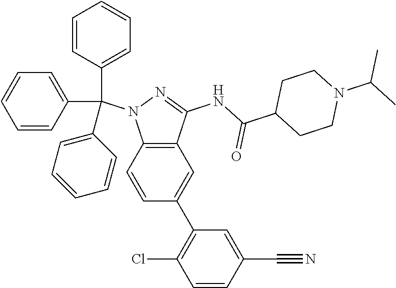 [SMILES: CC(C)N1CCC(C(=O)Nc2nn(C(c3ccccc3)(c3ccccc3)c3ccccc3)c3ccc(-c4cc(C#N)ccc4Cl)cc23)CC1]

Starting from N-[5-(2-chloro-5-cyanophenyl)-1-trityl-1H-indazol-3-yl]piperidine-4-carboxamide and acetone. Flash column chromatography (DCM/MeOH/33% aqueous ammonia95:5:0.5). Obtained 0.062 g (81% of yield). 1H NMR (500 MHz, DMSO-d₆) δ ppm 0.94 (d, J=6.41 Hz, 6H), 1.55-1.67 (m, 2H), 1.78 (d, J=11.90 Hz, 2H), 2.07-2.13 (m, 2H), 2.41 (m, 1H), 2.66 (m, 1H), 2.80 (d, J=9.30 Hz, 2H), 6.46 (d, J=8.85 Hz, 1H), 7.16 (d, J=8.85 Hz, 1H), 7.23-7.25 (m, 6H), 7.27-7.31 (m, 3H), 7.32-7.38 (m, 6H), 7.77 (d, J=8.39 Hz, 1H), 7.82 (s, 1H), 7.84 (dd, J=8.24, 1.98 Hz, 1H), 7.88 (s, 1H), 10.47 (s, 1H). HRMS (ESI) calcd for C42H39ClN5O [M+H]⁺ 664.2838, found 664.2860.

### N-[5-(2-Chloro-5-cyanophenyl)-1-trityl-1H-indazol-3-yl]-1-propylpiperidine-4-carboxamide [(XIII c), R1=2-chloro-5-cyanophenyl, R2=1-propylpiperidine, R9=1,1′,1″-methanetriyltribenzene]

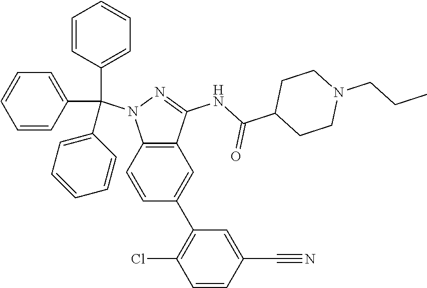 [SMILES: CCCN1CCC(C(=O)Nc2nn(C(c3ccccc3)(c3ccccc3)c3ccccc3)c3ccc(-c4cc(C#N)ccc4Cl)cc23)CC1]

Starting from N-[5-(2-chloro-5-cyanophenyl)-1-trityl-1H-indazol-3-yl]piperidine-4-carboxamide and propionaldehyde. Flash column chromatography (DCM/MeOH/33% aqueous ammonia 95:5:0.5). Obtained 0.064 g (83% of yield). ¹H NMR (500 MHz, DMSO-d₆) δ ppm 0.83 (t, J=7.32 Hz, 3H), 1.40 (m, 2H), 1.58-1.68 (m, 2H), 1.77 (d, J=11.44 Hz, 2H), 1.82-1.87 (m, 2H), 2.19 (t, J=7.17 Hz, 2H), 2.43 (m, 1H), 2.86 (d, J=10.37 Hz, 2H), 6.46 (d, J=9.15 Hz, 1H), 7.16 (d, J=9.30 Hz, 1H), 7.23-7.25 (m, 6H), 7.27-7.31 (m, 3H), 7.33-7.36 (m, 6H), 7.77 (d, J=8.39 Hz, 1H), 7.81-7.86 (m, 2H), 7.88 (s, 1H), 10.48 (s, 1H). HRMS (ESI) calcd for C42H39ClN5O [M+H]⁺ 664.2838, found 664.2851.

### N-[5-(2-Chloro-5-cyanophenyl)-1-trityl-1H-indazol-3-yl]-1-pentylpiperidine-4-carboxamide [(XIII c), R1=2-chloro-5-cyanophenyl, R2=1-pentylpiperidine, R9=1,1′,1″-methanetriyltribenzene]

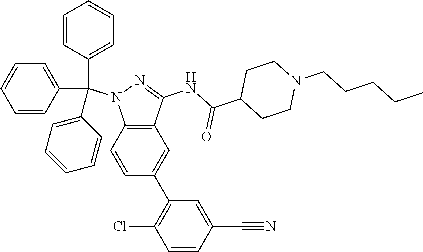 [SMILES: CCCCCN1CCC(C(=O)Nc2nn(C(c3ccccc3)(c3ccccc3)c3ccccc3)c3ccc(-c4cc(C#N)ccc4Cl)cc23)CC1]

Starting from N-[5-(2-chloro-5-cyanophenyl)-1-trityl-1H-indazol-3-yl]piperidine-4-carboxamide and pentanal. Flash column chromatography (DCM/MeOH/33% aqueous ammonia95:5:0.5). Obtained 0.076 g (95% of yield). 1H NMR (500 MHz, DMSO-d₆) δ ppm 0.86 (t, J=7.17 Hz, 3H), 1.15-1.33 (m, 4H), 1.40 (m, 2H), 1.57-1.69 (m, 2H), 1.73-1.85 (m, 4H), 2.22 (t, J=6.94 Hz, 2H), 2.43 (m, 1H), 2.87 (d, J=10.22 Hz, 2H), 6.45 (d, J=8.85 Hz, 1H), 7.15 (d, J=9.30 Hz, 1H), 7.23-7.25 (m, 6H), 7.28-7.31 (m, 3H), 7.33-7.36 (m, 6H), 7.76 (d, J=8.24 Hz, 1H), 7.82-7.87 (m, 2H), 7.88 (s, 1H), 10.48 (s, 1H). HRMS (ESI) calcd for C44H43ClN5O [M+H]⁺ 692.3151, found 692.3168.

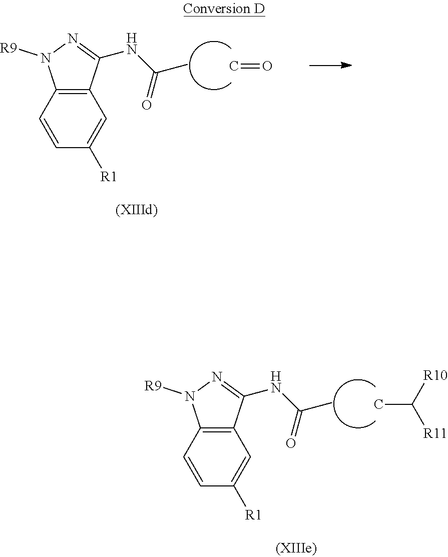

The compounds were prepared according to the same procedure reported for conversion B.

### N-[5-(2,6-Dichlorophenyl)-1-trityl-1H-indazol-3-yl]-4-(methylamino)cyclohexanecarboxamide [(XIIIe), R1=2,6-dichlorophenyl, R2=(methylamino)cyclohexane, R9=1,1′,1″-methanetriyltribenzene]

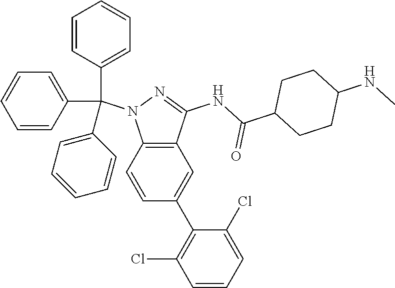 [SMILES: CNC1CCC(C(=O)Nc2nn(C(c3ccccc3)(c3ccccc3)c3ccccc3)c3ccc(-c4c(Cl)cccc4Cl)cc23)CC1]

Starting from N-[5-(2,6-Dichlorophenyl)-1-trityl-1H-indazol-3-yl]-4-oxocyclohexanecarboxamide and 2M methylamine in THF. Flash column chromatography (DCM/MeOH/33% aqueous ammonia 90:10:1). Obtained 0.047 (77% of yield). ¹H NMR (500 MHz, DMSO-d₆) δ ppm 1.38-1.49 (m, 4H), 1.64-1.68 (m, 2H), 1.77-1.90 (m, 2H), 2.21 (s, 3H), 6.46 (d, J=9.00 Hz, 1H), 6.93 (d, J=8.85 Hz, 1H), 7.25-7.30 (m, 9H), 7.33-7.36 (m, 6H), 7.41 (dd, J=8.54, 7.63 Hz, 1H), 7.55 (d, J=7.93 Hz, 2H), 7.62 (s, 1H), 10.35 (s, 1H). HRMS (ESI) calcd for C40H37Cl2N4ONa [M+Na+H]681.2158, found 681.2155.

### N-[5-(2,6-Dichlorophenyl)-1-trityl-1H-indazol-3-yl]-3-(methylamino)cyclobutanecarboxamide [(XIIIe), R1=2,6-dichlorophenyl, R2=(methylamino)cyclobutane, R9=1,1′,1″-methanetriyltribenzene]

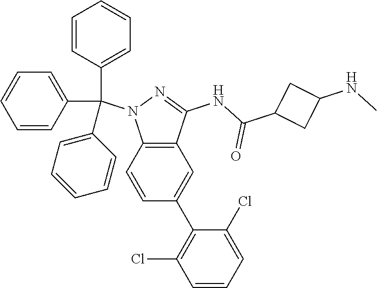 [SMILES: CNC1CC(C(=O)Nc2nn(C(c3ccccc3)(c3ccccc3)c3ccccc3)c3ccc(-c4c(Cl)cccc4Cl)cc23)C1]

Starting from N-[5-(2,6-dichlorophenyl)-1-trityl-1H-indazol-3-yl]-3-oxocyclobutanecarboxamide and 2M methylamine in THF. Flash column chromatography (DCM/MeOH/33% aqueous ammonia 95:5:0.5). Obtained 0.026 g (51% of yield). LCMS (ESI) m/z 631 (M+H)⁺.

### N-[5-(2-Chloro-5-cyanophenyl)-1-trityl-1H-indazol-3-yl]-3-(methylamino)cyclobutanecarboxamide [(XIIIe), R1=2-chloro-5-cyanophenyl, R2=(methylamino)cyclobutane, R9=1,1′,1″-methanetriyltribenzene]

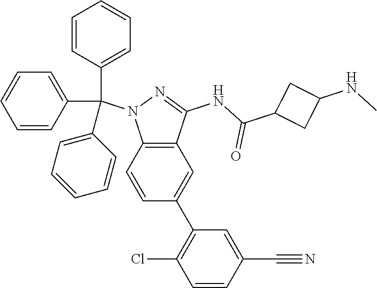 [SMILES: CNC1CC(C(=O)Nc2nn(C(c3ccccc3)(c3ccccc3)c3ccccc3)c3ccc(-c4cc(C#N)ccc4Cl)cc23)C1]

Starting from N-[5-(2-chloro-5-cyanophenyl)-1-trityl-1H-indazol-3-yl]-3-oxocyclobutanecarboxamide and 2M methylamine in THF. Flash column chromatography (DCM/MeOH 85:15). Obtained 0.012 g (59% of yield). LCMS (ESI) m/z 622 (M+H)⁺.

### N-[5-(2-Chloro-5-cyanophenyl)-1-trityl-1H-indazol-3-yl]-3-(dimethylamino)cyclobutanecarboxamide [(XIIIe), R1=2-chloro-5-cyanophenyl, R2=(dimethylamino)cyclobutane, R9=1,1′,1″-methanetriyltribenzene]

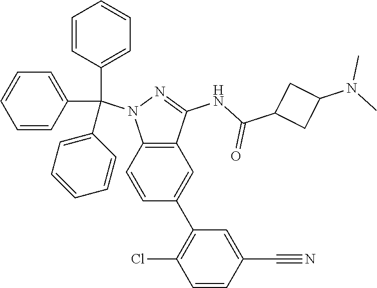 [SMILES: CN(C)C1CC(C(=O)Nc2nn(C(c3ccccc3)(c3ccccc3)c3ccccc3)c3ccc(-c4cc(C#N)ccc4Cl)cc23)C1]

Starting from N-[5-(2-chloro-5-cyanophenyl)-1-trityl-1H-indazol-3-yl]-3-oxocyclobutanecarboxamide and 2M dimethylamine in THF. Flash column chromatography (DCM/MeOH 85:15). Obtained 0.028 g (53% of yield). LCMS (ESI) m/z 636 (M+H)⁺.

### N-[5-(2-Chloro-5-cyanophenyl)-1-trityl-1H-indazol-3-yl]-3-(propan-2-ylamino)cyclobutanecarboxamide [(XIIIe), R1=2-chloro-5-cyanophenyl, R2=(propan-2-ylamino)cyclobutane, R9=methanetriyltribenzene]

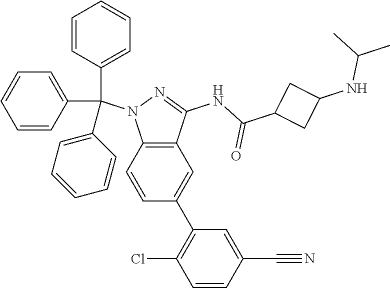 [SMILES: CC(C)NC1CC(C(=O)Nc2nn(C(c3ccccc3)(c3ccccc3)c3ccccc3)c3ccc(-c4cc(C#N)ccc4Cl)cc23)C1]

Starting from N-[5-(2-chloro-5-cyanophenyl)-1-trityl-1H-indazol-3-yl]-3-oxocyclobutanecarboxamide and isopropylamine. Flash column chromatography (DCM/MeOH 90:10). Obtained 0.044 g (82% of yield). ¹H NMR (500 MHz, DMSO-d₆) δ ppm 0.93 (d, J=6.10 Hz, 6H), 1.83-2.02 (m, 2H), 2.31-2.41 (m, 2H), 2.75 (m, 1H), 2.91 (m, 1H), 3.17 (m, 1H), 6.47 (d, J=8.85 Hz, 1H), 7.16 (d, J=8.85 Hz, 1H), 7.24 (d, J=7.63 Hz, 6H), 7.28-7.30 (m, 3H), 7.33-7.36 (m, 6H), 7.77 (d, J=8.24 Hz, 1H), 7.83-7.91 (m, 3H), 10.50 (s, 1H). HRMS (ESI) calcd for C41H37ClN5O [M+H]⁺ 650.2861, found 650.2695.

### N-[5-(2-Chloro-5-cyanophenyl)-1-trityl-1H-indazol-3-yl]-3-(morpholin-4-yl)cyclobutanecarboxamide [(XIIIe), R1=2-chloro-5-cyanophenyl, R2=(morpholin-4-yl)cyclobutane, R9=1,1′,1″-methanetriyltribenzene]

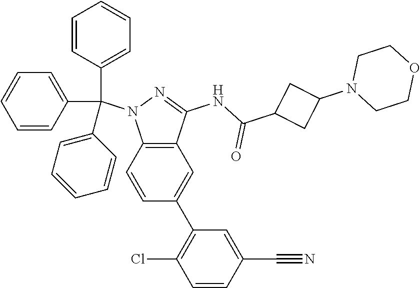 [SMILES: N#Cc1ccc(Cl)c(-c2ccc3c(c2)c(NC(=O)C2CC(N4CCOCC4)C2)nn3C(c2ccccc2)(c2ccccc2)c2ccccc2)c1]

Starting from N-[5-(2-chloro-5-cyanophenyl)-1-trityl-1H-indazol-3-yl]-3-oxocyclobutanecarboxamide and morpholine. Flash column chromatography (DCM/MeOH 95:5). Obtained 0.055 g (98% of yield). ¹H NMR (500 MHz, DMSO-d₆) δ ppm 1.96-2.06 (m, 2H), 2.12-2.30 (m, 6H), 2.61 (m, 1H), 2.98 (m, 1H), 3.54 (t, J=4.04 Hz, 4H), 6.47 (d, J=9.00 Hz, 1H), 7.16 (d, J=8.69 Hz, 1H), 7.25 (d, J=7.78 Hz, 6H), 7.28-7.30 (m, 3H), 7.33-7.36 (m, 6H), 7.77 (d, J=8.24 Hz, 1H), 7.84 (d, J=2.14 Hz, 1H), 7.87-7.91 (m, 2H), 10.54 (s, 1H). HRMS (ESI) calcd for C42H37ClN5O2 [M+H]⁺ 678.2631, found 678.2632.

### N-[5-(2-Chloro-5-cyanophenyl)-1-trityl-1H-indazol-3-yl]-3-[(2,2,2-trifluoroethyl)amino]cyclobutanecarboxamide [(XIIIe), R1=2-chloro-5-cyanophenyl, R2=[(2,2,2-trifluoroethyl)amino]cyclobutane, R9=1,1′,1″-methanetriyltribenzene]

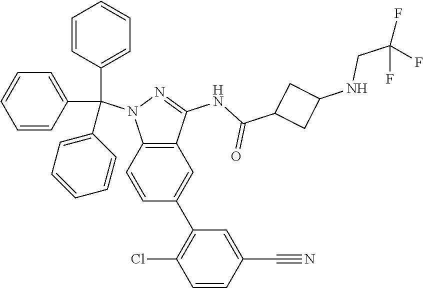 [SMILES: N#Cc1ccc(Cl)c(-c2ccc3c(c2)c(NC(=O)C2CC(NCC(F)(F)F)C2)nn3C(c2ccccc2)(c2ccccc2)c2ccccc2)c1]

Starting from N-[5-(2-chloro-5-cyanophenyl)-1-trityl-1H-indazol-3-yl]-3-oxocyclobutanecarboxamide and 2,2,2-trifluoro-ethylamine. Flash column chromatography (DCM/EtOAc 90:10). Obtained 0.048 g (85% of yield). 1H NMR (500 MHz, DMSO-d₆) δ ppm 1.90-1.97 (m, 2H), 2.33-2.36 (m, 2H), 2.64 (m, 1H), 2.86 (m, 1H), 3.14 (m, 2H), 6.47 (d, J=8.85 Hz, 1H), 7.16 (d, J=8.85 Hz, 1H), 7.24 (d, J=7.63 Hz, 6H), 7.28-7.30 (m, 3H), 7.33-7.36 (m, 6H), 7.77 (d, J=8.39 Hz, 1H), 7.84 (d, J=1.98 Hz, 1H), 7.86-7.89 (m, 2H), 10.47 (s, 1H). HRMS (ESI) calcd for C40H32ClF3N5O [M+H]⁺ 712.2061, found 712.2064.

### N-[5-(2-Chloro-5-cyanophenyl)-1-trityl-1H-indazol-3-yl]-3-[(2-fluoroethyl)amino]cyclobutanecarboxamide [(XIIIe), R1=2-chloro-5-cyanophenyl, R2=[(2-fluoroethyl)amino]cyclobutane, R9=methanetriyltribenzene]

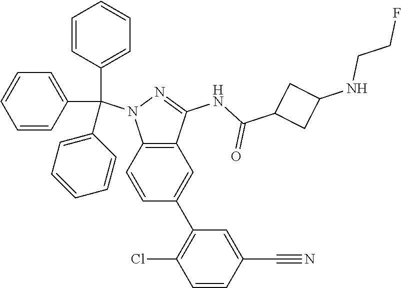 [SMILES: N#Cc1ccc(Cl)c(-c2ccc3c(c2)c(NC(=O)C2CC(NCCF)C2)nn3C(c2ccccc2)(c2ccccc2)c2ccccc2)c1]

Starting from N-[5-(2-chloro-5-cyanophenyl)-1-trityl-1H-indazol-3-yl]-3-oxocyclobutanecarboxamide and 2-fluoro-ethylamine. Flash column chromatography (DCM/MeOH 97:3). Obtained 0.045 g (84% of yield). ¹H NMR (500 MHz, DMSO-d₆) δ ppm 1.87-2.08 (m, 2H), 2.29-2.41 (m, 2H), 2.70-2.81 (m, 2H), 2.91 (m, 1H), 3.16 (m, 1H), 4.35-4.47 (m, 2H), 6.47 (d, J=9.00 Hz, 1H), 7.16 (d, J=9.00 Hz, 1H), 7.24 (d, J=7.47 Hz, 6H), 7.28-7.31 (m, 3H), 7.33-7.36 (m, 6H), 7.77 (d, J=8.24 Hz, 1H), 7.84 (d, J=2.14 Hz, 1H), 7.86-7.89 (m, 2H), 10.50 (s, 1H). HRMS (ESI) calcd for C40H34ClFN5O [M+H]⁺ 654.2431, found 654.2447.

### N-[5-(2-Chloro-5-cyanophenyl)-1-trityl-1H-indazol-3-yl]-3-(ethylamino)cyclobutanecarboxamide [(XIIIe), R1=2-chloro-5-cyanophenyl, R2=(ethylamino)cyclobutane, R9=1,1′,1″-methanetriyltribenzene]

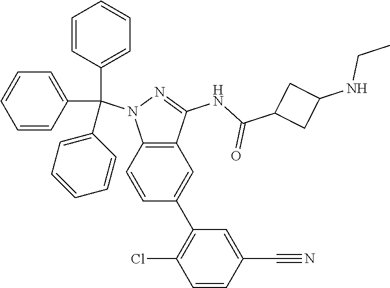 [SMILES: CCNC1CC(C(=O)Nc2nn(C(c3ccccc3)(c3ccccc3)c3ccccc3)c3ccc(-c4cc(C#N)ccc4Cl)cc23)C1]

Starting from N-[5-(2-chloro-5-cyanophenyl)-1-trityl-1H-indazol-3-yl]-3-oxocyclobutanecarboxamide and ethylamine hydrochloride. Flash column chromatography (DCM/MeOH 90:10). Obtained 0.038 g (73% of yield). ¹H NMR (500 MHz, DMSO-d₆) δ ppm 0.96 (t, J=7.17 Hz, 3H), 1.88-2.01 (m, 2H), 2.28-2.39 (m, 2H), 2.47 (m, 2H), 2.92 (m, 1H), 3.11 (m, 1H), 6.47 (d, J=9.00 Hz, 1H), 7.15 (d, J=8.69 Hz, 1H), 7.24 (d, J=7.47 Hz, 6H), 7.28-7.30 (m, 3H), 7.33-7.36 (m, 6H), 7.77 (d, J=8.39 Hz, 1H), 7.84 (d, J=1.98 Hz, 1H), 7.86-7.89 (m, 2H), 10.51 (s, 1H). HRMS (ESI) calcd for C40H35ClN5O [M+H]⁺ 636.2525, found 636.2548.

### N-[5-(2-Chloro-5-cyanophenyl)-1-trityl-1H-indazol-3-yl]-3-(cyclobutylamino)cyclobutanecarboxamide [(XIIIe), R1=2-chloro-5-cyanophenyl, R2=(cyclobutylamino)cyclobutane, R9=1,1′,1″-methanetriyltribenzene]

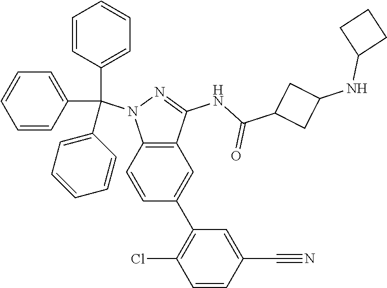 [SMILES: N#Cc1ccc(Cl)c(-c2ccc3c(c2)c(NC(=O)C2CC(NC4CCC4)C2)nn3C(c2ccccc2)(c2ccccc2)c2ccccc2)c1]

Starting from N-[5-(2-chloro-5-cyanophenyl)-1-trityl-1H-indazol-3-yl]-3-oxocyclobutanecarboxamide and cyclobutylamine. Flash column chromatography (DCM/MeOH 90:10). Obtained 0.051 g (94% of yield). ¹H NMR (500 MHz, DMSO-d₆) δ ppm 1.48-1.70 (m, 4H), 1.88-2.07 (m, 4H), 2.28 (m, 2H), 2.89 (m, 1H), 3.02-3.21 (m, 2H), 6.47 (d, J=9.00 Hz, 1H), 7.16 (d, J=9.00 Hz, 1H), 7.25 (d, J=7.63 Hz, 6H), 7.27-7.32 (m, 3H), 7.32-7.38 (m, 6H), 7.77 (d, J=8.24 Hz, 1H), 7.84 (d, J=2.13 Hz, 1H), 7.86-7.89 (m, 2H), 10.47 (s, 1H). HRMS (ESI) calcd for C42H37ClN5O [M+H]⁺ 662.2681, found 662.2692.

### N-[5-(2-Chloro-5-cyanophenyl)-1-trityl-1H-indazol-3-yl]-3-(cyclopentylamino)cyclobutanecarboxamide [(XIIIe), R1=2-chloro-5-cyanophenyl, R2=(cyclopentylamino)cyclobutane, R9=methanetriyltribenzene]

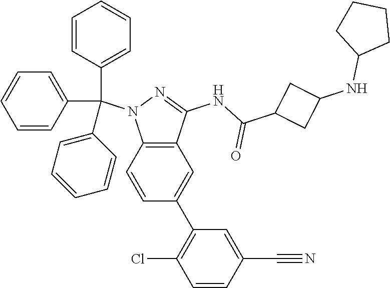 [SMILES: N#Cc1ccc(Cl)c(-c2ccc3c(c2)c(NC(=O)C2CC(NC4CCCC4)C2)nn3C(c2ccccc2)(c2ccccc2)c2ccccc2)c1]

Starting from N-[5-(2-chloro-5-cyanophenyl)-1-trityl-1H-indazol-3-yl]-3-oxocyclobutanecarboxamide and cyclopentylamine. Flash column chromatography (DCM/MeOH 90:10). Obtained 0.055 g (92% of yield). ¹H NMR (500 MHz, DMSO-d₆) δ ppm 1.27-1.34 (m, 2H), 1.42-1.51 (m, 2H), 1.58-1.65 (m, 2H), 1.67-1.74 (m, 2H), 1.95-2.04 (m, 2H), 2.36-2.42 (m, 2H), 2.94-3.07 (m, 2H), 3.16-3.25 (m, 1H), 6.51 (d, J=9.00 Hz, 1H), 7.20 (d, J=8.85 Hz, 1H), 7.27 (d, J=7.78 Hz, 6H), 7.31-7.34 (m, 3H), 7.37-7.40 (m, 6H), 7.81 (d, J=8.24 Hz, 1H), 7.88 (d, J=2.14 Hz, 1H), 7.89-7.92 (m, 2H), 10.55 (s, 1H). HRMS (ESI) calcd for C43H39ClN5O [M+H]⁺ 676.2838, found 676.2845.

### N-[5-(2-Chloro-5-cyanophenyl)-1-trityl-1H-indazol-3-yl]-3-(cyclopropylamino)cyclobutanecarboxamide [(XIIIe), R1=2-chloro-5-cyanophenyl, R2=(cyclopropylamino)cyclobutane, R9=methanetriyltribenzene]

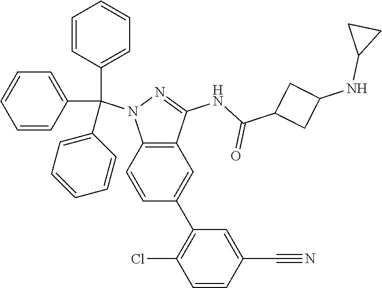 [SMILES: N#Cc1ccc(Cl)c(-c2ccc3c(c2)c(NC(=O)C2CC(NC4CC4)C2)nn3C(c2ccccc2)(c2ccccc2)c2ccccc2)c1]

Starting from N-[5-(2-chloro-5-cyanophenyl)-1-trityl-1H-indazol-3-yl]-3-oxocyclobutanecarboxamide and cyclopropylamine. Flash column chromatography (DCM/MeOH 94:6). Obtained 0.048 g (94% of yield). ¹H NMR (500 MHz, DMSO-d₆) δ ppm 0.15-0.17 (m, 2H), 0.28-0.33 (m, 2H), 1.90-2.06 (m, 3H), 2.28-2.36 (m, 2H), 2.90 (m, 1H), 3.13 (m, 1H), 6.47 (d, J=8.85 Hz, 1H), 7.16 (d, J=8.54 Hz, 1H), 7.24 (d, J=7.24 Hz, 6H), 7.28-7.30 (m, 3H), 7.33-7.36 (m, 6H), 7.77 (d, J=8.39 Hz, 1H), 7.84 (d, J=2.14 Hz, 1H), 7.86-7.89 (m, 2H), 10.47 (s, 1H). HRMS (ESI) calcd for C41H35ClN5O [M+H]⁺ 648.2525, found 648.2545.

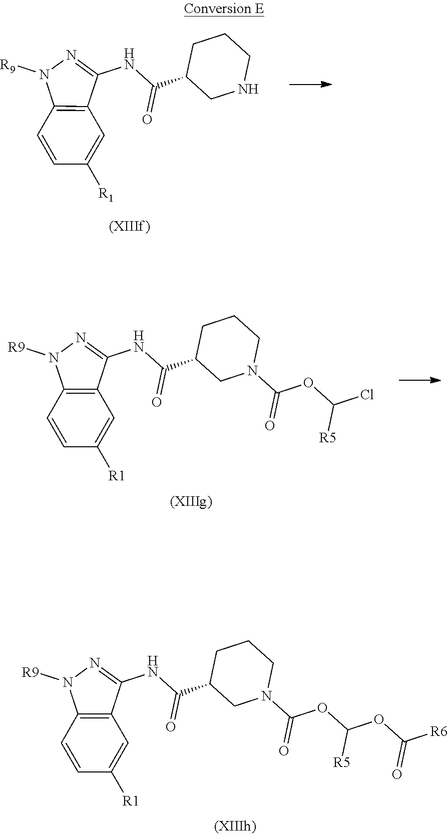

### Chloromethyl (3R)-3-{[5-(2-chloro-5-cyanophenyl)-1-trityl-1H-indazol-3-yl]carbamoyl}piperidine-1-carboxylate [(XIII g), R1=2-chloro-5-cyanophenyl, R2=chloromethyl piperidine-1-carboxylate, R9=1,1′,1″-methanetriyltribenzene]

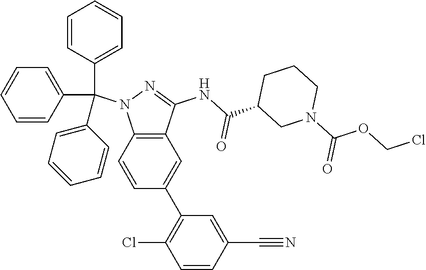 [SMILES: N#Cc1ccc(Cl)c(-c2ccc3c(c2)c(NC(=O)[C@@H]2CCCN(C(=O)OCCl)C2)nn3C(c2ccccc2)(c2ccccc2)c2ccccc2)c1]

To a solution of (3R)-N-[5-(2-chloro-5-cyanophenyl)-1-trityl-1H-indazol-3-yl]piperidine-3-carboxamide (0.4 g, 0.644 mmol) and N,N-diisopropylethylamine (0.228 mL, 1.288 mmol) in dry THF (10 mL), cooled at 0-5° C., a solution of chloromethyl chloroformate (0.063 mL, 0.708 mmol) in dry THF (5 mL) was added dropwise. The reaction was stirred at 0-5° C. for 1 h, then was diluted with EtOAc (20 mL) and washed with cold water (20 mL) and brine (20 mL). The organic phase was separated, dried with anhydrous sodium sulphate, filtered and evaporated to dryness to give chloromethyl (3R)-3-{[5-(2-chloro-5-cyanophenyl)-1-trityl-1H-indazol-3-yl]carbamoyl}piperidine-1-carboxylate (0.45 g. 98% of yield) as white solid. It was used for the next step without purification. LCMS (ESI) m/z 714(M+H)⁺.

According to this same methodology, starting from the appropriate reagents, the following compounds were obtained:

### 1-Chloroethyl (3R)-3-{[5-(2-chloro-5-cyanophenyl)-1-trityl-1H-indazol-3-yl]carbamoyl}piperidine-1-carboxylate [(XIII g), R1=2-chloro-5-cyanophenyl, R2=1-chloroethyl piperidine-1-carboxylate, R9=1,1′,1″-methanetriyltribenzene]

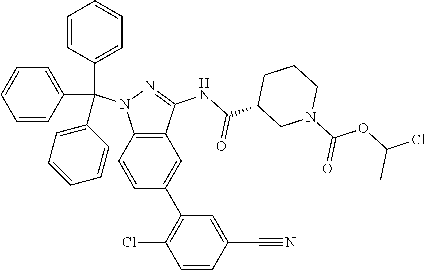 [SMILES: CC(Cl)OC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from (3R)-N-[5-(2-chloro-5-cyanophenyl)-1-trityl-1H-indazol-3-yl]piperidine-3-carboxamide and 1-chloroethyl chloroformate. Obtained 1.2 g (99% of yield). LCMS (ESI) m/z 728 (M+H)⁺.

### 1-Chloro-2-methylpropyl (3R)-3-{[5-(2-chloro-5-cyanophenyl)-1-trityl-1H-indazol-3-yl]carbamoyl}piperidine-1-carboxylate [(XIII g), R1=2-chloro-5-cyanophenyl, R2=1-chloro-2-methylpropyl piperidine-1-carboxylate, R9=1,1′,1″-methanetriyltribenzene]

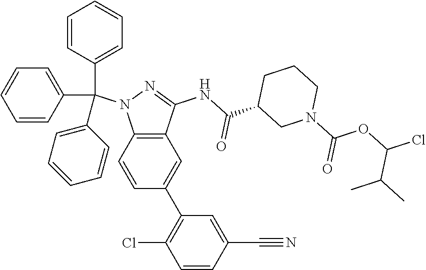 [SMILES: CC(C)C(Cl)OC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from (3R)-N-[5-(2-chloro-5-cyanophenyl)-1-trityl-1H-indazol-3-yl]piperidine-3-carboxamide and 1-chloro-2-methylpropyl chloroformate. Obtained 0.351 g (96% of yield). LCMS (ESI) m/z 756 (M+H)⁺.

### 1-Chloropropyl (3R)-3-{[5-(2-chloro-5-cyanophenyl)-1-trityl-1H-indazol-3-yl]carbamoyl}piperidine-1-carboxylate [(XIII g), R1=2-chloro-5-cyanophenyl, R2=1-chloropropyl piperidine-1-carboxylate, R9=1,1′,1″-methanetriyltribenzene]

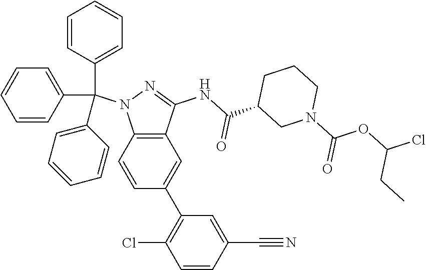 [SMILES: CCC(Cl)OC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from (3R)-N-[5-(2-chloro-5-cyanophenyl)-1-trityl-1H-indazol-3-yl]piperidine-3-carboxamide and 1-chloropropyl chloroformate. Obtained 0.675 g (94% of yield). LCMS (ESI) m/z 742 (M+H)⁺.

### 1-[(2,2-Dimethylpentanoyl)oxy]ethyl (3R)-3-{[5-(2-chloro-5-cyanophenyl)-1-trityl-1H-indazol-3-yl]carbamoyl}-piperidine-1-carboxylate [(XIIIh), R1=2-chloro-5-cyanophenyl, R2=1-[(2,2-dimethylpentanoyl)oxy]ethyl piperidine-1-carboxylate, R9=1,1′,1″-methanetriyltribenzene]

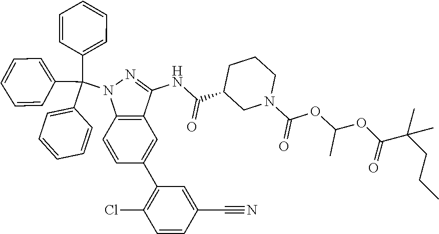 [SMILES: CCCC(C)(C)C(=O)OC(C)OC(=O)N1CCC[C@@H](C(=O)Nc2nn(C(c3ccccc3)(c3ccccc3)c3ccccc3)c3ccc(-c4cc(C#N)ccc4Cl)cc23)C1]

To a solution of 1-chloroethyl (3R)-3-{[5-(2-chloro-5-cyanophenyl)-1-trityl-1H-indazol-3-yl]carbamoyl}piperidine-1-carboxylate (0.2 g, 0.275 mmol) in dry DMF (3 mL), 2,2-dimethyl-pentanoic acid (0.078 mL, 0.55 mmol), cesium carbonate (0.179 g, 0.55 mmol) and sodium iodide (0.062 g, 0.413 mmol) were added. The mixture was stirred at room temperature for 24 h, then was diluted with EtOAc (20 mL) and washed with cold water (20 mL) and brine (20 mL). The organic phase was separated, dried with anhydrous sodium sulphate, filtered and evaporated to dryness. The crude was purified by flash column chromatography (Hexane/EtOAc 70:30) to furnish 1-[(2,2-dimethylpentanoyl)oxy]ethyl (3R)-3-{[5-(2-chloro-5-cyanophenyl)-1-trityl-1H-indazol-3-yl]carbamoyl}-piperidine-1-carboxylate (0.145 g, 64% of yield) as white solid. ¹H NMR (500 MHz, DMSO-d₆) δ ppm 0.71-0.85 (m, 4H), 1.02-1.18 (m, 8H), 1.33-141 (m, 6H), 1.61-1.74 (m, 2H), 1.95-2.04 (m, 1H), 2.60 (m, 1H), 2.86-3.02 (m, 2H), 3.80-3.94 (m, 1H), 6.46 (d, J=9.00 Hz, 1H), 6.62 (m, 1H), 7.16 (d, J=9.15 Hz, 1H), 7.24 (d, J=7.63 Hz, 6H), 7.28-7.31 (m, 3H), 7.32-7.36 (m, 6H), 7.76 (d, J=8.39 Hz, 1H), 7.82-7.87 (m, 3H), 10.70 (br. s., 1H). HRMS (ESI) calcd for C49H49ClN5O5 [M+H]⁺ 822.3417, found 822.3419.

### According to this same methodology, starting from the appropriate intermediates and reagents, the following compounds were obtained: 1-[(2-Methylpropanoyl)oxy]ethyl (3R)-3-{[5-(2-chloro-5-cyanophenyl)-1-trityl-1H-indazol-3-yl]carbamoyl}-piperidine-1-carboxylate [(XIIIh), R1=2-chloro-5-cyanophenyl, R2=1-[(2-methylpropanoyl)oxy]ethyl piperidine-1-carboxylate, R9=1,1′,1″-methanetriyltribenzene]

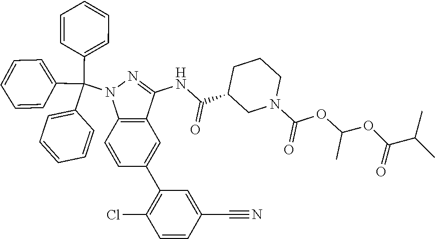 [SMILES: CC(OC(=O)C(C)C)OC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from 1-chloroethyl (3R)-3-{[5-(2-chloro-5-cyanophenyl)-1-trityl-1H-indazol-3-yl]carbamoyl}piperidine-1-carboxylate and 2-methylpropanoic acid. Flash column chromatography (Hexane/EtOAc 70:30). Obtained 0.251 g (70% of yield). ¹H NMR (500 MHz, DMSO-d₆) δ ppm 1.04 (d, J=6.86 Hz, 6H), 1.31-1.45 (m, 4H), 1.62-1.74 (m, 2H), 1.95-2.02 (m, 2H), 2.60 (m, 1H), 2.82-3.04 (m, 2H), 3.76-3.97 (m, 1H), 6.47 (d, J=8.85 Hz, 1H), 6.63 (m, 1H), 7.16 (d, J=9.15 Hz, 1H), 7.23 (d, J=7.63 Hz, 6H), 7.28-7.31 (m, 3H), 7.33-7.36 (m, 6H), 7.76 (d, J=8.39 Hz, 1H), 7.83-7.88 (m, 3H), 10.65-10.74 (m, 1H). HRMS (ESI) calcd for C46H43ClN5O5 [M+H]⁺ 780.3092, found 780.3085.

### (Acetyloxy)methyl (3R)-3-{[5-(2-chloro-5-cyanophenyl)-1-trityl-1H-indazol-3-yl]carbamoyl}piperidine-1-carboxylate [(XIIIh), R1=2-chloro-5-cyanophenyl, R2=(acetyloxy)methyl piperidine-1-carboxylate, R9=1,1′,1″-methanetriyltribenzene]

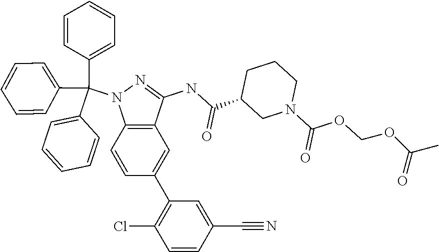 [SMILES: CC(=O)OCOC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from chloromethyl (3R)-3-{[5-(2-chloro-5-cyanophenyl)-1-trityl-1H-indazol-3-yl]carbamoyl}piperidine-1-carboxylate and acetic acid. Flash column chromatography (Hexane/EtOAc 70:30). Obtained 0.105 g (88% of yield). 1H NMR (500 MHz, DMSO-d₆) δ ppm 1.35-1.39 (m, 1H), 1.62-1.76 (m, 2H), 1.99-2.05 (m, 4H), 2.63 (m, 1H), 2.84-3.11 (m, 2H), 3.80-4.05 (m, 2H), 5.64 (s, 2H), 6.47 (d, J=9.00 Hz, 1H), 7.17 (d, J=8.85 Hz, 1H), 7.24 (d, J=7.63 Hz, 6H), 7.28-7.31 (m, 3H), 7.34-7.37 (m, 6H), 7.77 (d, J=8.39 Hz, 1H), 7.82-7.89 (m, 3H), 10.70 (br. s., 1H). HRMS (ESI) calcd for C43H37ClN5O5 [M+H]⁺ 738.2294, found 738.2299.

### [(2-Methylpropanoyl)oxy]methyl (3R)-3-{[5-(2-chloro-5-cyanophenyl)-1-trityl-1H-indazol-3-yl]carbamoyl}-piperidine-1-carboxylate [(XIIIh), R1=2-chloro-5-cyanophenyl, R2=[(2-methylpropanoyl)oxy]methyl piperidine-1-carboxylate, R9=1,1′,1″-methanetriyltribenzene]

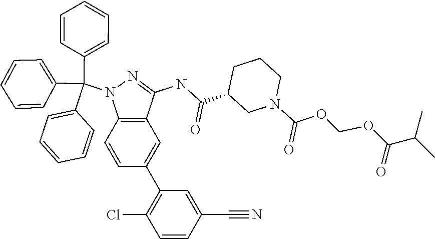 [SMILES: CC(C)C(=O)OCOC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from chloromethyl (3R)-3-{[5-(2-chloro-5-cyanophenyl)-1-trityl-1H-indazol-3-yl]carbamoyl}piperidine-1-carboxylate and 2-methylpropanoic acid. Flash column chromatography (Hexane/EtOAc 75:25). Obtained 0.074 g (81% of yield). LCMS (ESI) m/z 766 (M+H)⁺.

### [(2,2-Dimethylpropanoyl)oxy]methyl (3R)-3-{[5-(2-chloro-5-cyanophenyl)-1-trityl-1H-indazol-3-yl]carbamoyl}-piperidine-1-carboxylate [(XIIIh), R1=2-chloro-5-cyanophenyl, R2=[(2,2-dimethylpropanoyl)oxy]methyl piperidine-1-carboxylate, R9=1,1′,1″-methanetriyltribenzene]

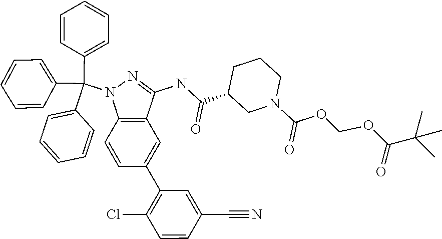 [SMILES: CC(C)(C)C(=O)OCOC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from chloromethyl (3R)-3-{[5-(2-chloro-5-cyanophenyl)-1-trityl-1H-indazol-3-yl]carbamoyl}piperidine-1-carboxylate and pivalic acid. Flash column chromatography (Hexane/EtOAc 75:25). Obtained 0.098 g (74% of yield). ¹H NMR (500 MHz, DMSO-d₆) δ ppm 1.12 (s, 9H), 1.29-1.40 (m, 1H), 1.57-1.77 (m, 2H), 1.93-2.03 (m, 1H), 2.60 (m, 1H), 2.88-3.15 (m, 2H), 3.77-3.98 (m, 2H), 5.66 (br. s., 2H), 6.47 (d, J=8.85 Hz, 1H), 7.17 (d, J=9.00 Hz, 1H), 7.23 (d, J=7.63 Hz, 6H), 7.28-7.31 (m, 3H), 7.33-7.36 (m, 6H), 7.76 (d, J=8.24 Hz, 1H), 7.81 (br. s., 1H), 7.83-7.87 (m, 1H), 7.88 (s, 1H), 10.58-10.84 (m, 1H). HRMS (ESI) calcd for C46H43ClN5O5Na [M+H+Na]⁺802.2766, found 802.2749.

### 1-(Acetyloxy)ethyl (3R)-3-{[5-(2-chloro-5-cyanophenyl)-1-trityl-1H-indazol-3-yl]carbamoyl}piperidine-1-carboxylate [(XIIIh), R1=2-chloro-5-cyanophenyl, R2=1-(acetyloxy)ethyl piperidine-1-carboxylate, R9=1,1′,1″-methanetriyltribenzene]

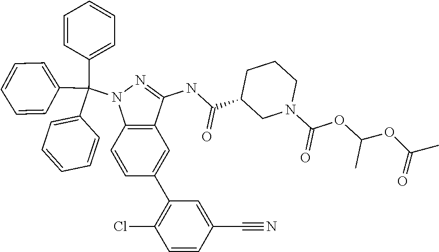 [SMILES: CC(=O)OC(C)OC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from 1-chloroethyl (3R)-3-{[5-(2-chloro-5-cyanophenyl)-1-trityl-1H-indazol-3-yl]carbamoyl}piperidine-1-carboxylate and acetic acid. Flash column chromatography (Hexane/EtOAc 70:30). Obtained 0.118 g (90% of yield). ¹H NMR (500 MHz, DMSO-d₆) δ ppm 1.32-1.45 (m, 4H), 1.63-1.76 (m, 2H), 1.96-2.04 (m, 4H), 2.63 (m, 1H), 2.93-3.08 (m, 2H), 3.81-4.00 (m, 2H), 6.48 (d, J=9.00 Hz, 1H), 6.63 (m, 1H), 7.18 (d, J=9.00 Hz, 1H), 7.25 (d, J=7.63 Hz, 6H), 7.29-7.32 (m, 3H), 7.35-7.38 (m, 6H), 7.78 (d, J=8.39 Hz, 1H), 7.83-7.88 (m, 2H), 7.89 (s, 1H), 10.71 (s, 1H). HRMS (ESI) calcd for C44H39ClN5O5Na [M+H+Na]⁺774.2453, found 774.2432.

### 1-[(2,2-Dimethylpropanoyl)oxy]ethyl (3R)-3-{[5-(2-chloro-5-cyanophenyl)-1-trityl-1H-indazol-3-yl]carbamoyl}-piperidine-1-carboxylate [(XIIIh), R1=2-chloro-5-cyanophenyl, R2=1-[(2,2-dimethylpropanoyl)oxy]ethyl piperidine-1-carboxylate, R9=1,1′,1″-methanetriyltribenzene]

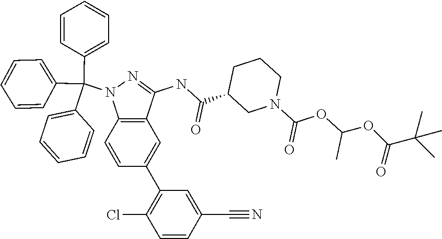 [SMILES: CC(OC(=O)N1CCC[C@@H](C(=O)Nc2nn(C(c3ccccc3)(c3ccccc3)c3ccccc3)c3ccc(-c4cc(C#N)ccc4Cl)cc23)C1)OC(=O)C(C)(C)C]

Starting from 1-chloroethyl (3R)-3-{[5-(2-chloro-5-cyanophenyl)-1-trityl-1H-indazol-3-yl]carbamoyl}piperidine-1-carboxylate and pivalic acid acid. Flash column chromatography (Hexane/EtOAc 75:25). Obtained 0.086 g (72% of yield). LCMS (ESI) m/z 794 (M+H)⁺.

### 2-Methyl-1-[(2-methylpropanoyl)oxy]propyl (3R)-3-{[5-(2-chloro-5-cyanophenyl)-1-trityl-1H-indazol-3-yl]carbamoyl}piperidine-1-carboxylate [(XIIIh), R1=2-chloro-5-cyanophenyl, R2=2-methyl-1-[(2-methylpropanoyl)oxy]propyl piperidine-1-carboxylate, R9=1,1′,1″-methanetriyltribenzene]

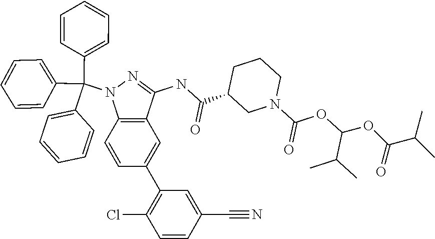 [SMILES: CC(C)C(=O)OC(OC(=O)N1CCC[C@@H](C(=O)Nc2nn(C(c3ccccc3)(c3ccccc3)c3ccccc3)c3ccc(-c4cc(C#N)ccc4Cl)cc23)C1)C(C)C]

Starting from 1-chloro-2-methylpropyl (3R)-3-{[5-(2-chloro-5-cyanophenyl)-1-trityl-1H-indazol-3-yl]carbamoyl}-piperidine-1-carboxylate and 2-methylpropanoic acid. Flash column chromatography (Hexane/EtOAc 75:25). Obtained 0.157 g (65% of yield). ¹H NMR (500 MHz, DMSO-d₆) δ ppm 0.90 (m, 6H), 1.07 (m, 6H), 1.31-1.41 (m, 1H), 1.63-1.75 (m, 2H), 1.93-2.03 (m, 2H), 2.63 (m, 1H), 2.91-3.01 (m, 1H), 3.78-3.94 (m, 1H), 3.99-4.04 (m, 1H), 6.43 (d, J=4.73 Hz, 1H), 6.47 (d, J=9.15 Hz, 1H), 7.17 (d, J=8.69 Hz, 1H), 7.25 (d, J=7.78 Hz, 6H), 7.29-7.32 (m, 3H), 7.35-7.38 (m, 6H), 7.78 (d, J=8.39 Hz, 1H), 7.85-7.89 (m, 3H), 10.69 (br. s., 1H). HRMS (ESI) calcd for C48H47ClN5O5 [M+H]⁺ 808.3260, found 808.3275.

### 1-[(2,2-Dimethylpropanoyl)oxy]-2-methylpropyl (3R)-3-{[5-(2-chloro-5-cyanophenyl)-1-trityl-1H-indazol-3-yl]carbamoyl}piperidine-1-carboxylate [(XIIIh), R1=2-chloro-5-cyanophenyl, R2=1-[(2,2-dimethylpropanoyl)oxy]-2-methylpropyl piperidine-1-carboxylate, R9=1,1′,1″-methanetriyltribenzene]

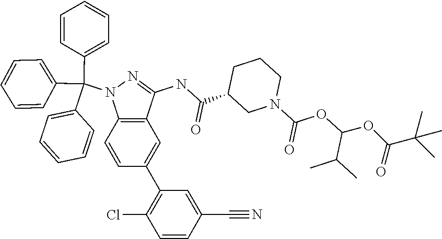 [SMILES: CC(C)C(OC(=O)N1CCC[C@@H](C(=O)Nc2nn(C(c3ccccc3)(c3ccccc3)c3ccccc3)c3ccc(-c4cc(C#N)ccc4Cl)cc23)C1)OC(=O)C(C)(C)C]

Starting from 1-chloro-2-methylpropyl (3R)-3-{[5-(2-chloro-5-cyanophenyl)-1-trityl-1H-indazol-3-yl]carbamoyl}-piperidine-1-carboxylate and pivalic acid. Flash column chromatography (Hexane/EtOAc 75:25). Obtained 0.129 g (57% of yield). LCMS (ESI) m/z 822 (M+H)⁺.

### 1-{[(1-Methylcyclohexyl)carbonyl]oxy}ethyl (3R)-3-{[5-(2-chloro-5-cyanophenyl)-1-trityl-1H-indazol-3-yl]carbamoyl}piperidine-1-carboxylate [(XIIIh), R1=2-chloro-5-cyanophenyl, R2=1-{[(1-methylcyclohexyl)carbonyl]oxy}ethyl piperidine-1-carboxylate, R9=1,1′,1″-methanetriyltribenzene]

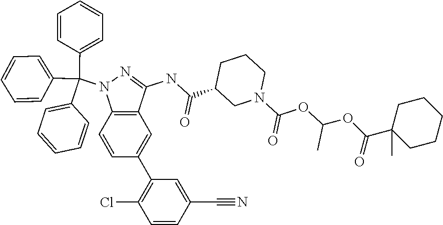 [SMILES: CC(OC(=O)N1CCC[C@@H](C(=O)Nc2nn(C(c3ccccc3)(c3ccccc3)c3ccccc3)c3ccc(-c4cc(C#N)ccc4Cl)cc23)C1)OC(=O)C1(C)CCCCC1]

Starting from 1-chloroethyl (3R)-3-{[5-(2-chloro-5-cyanophenyl)-1-trityl-1H-indazol-3-yl]carbamoyl}piperidine-1-carboxylate and 1-methyl-cyclohexanecarboxylic acid. Flash column chromatography (Hexane/EtOAc 70:30). Obtained 0.113 g (49% of yield). ¹H NMR (500 MHz, DMSO-d₆) δ ppm 1.02-1.25 (m, 10H), 1.34-1.49 (m, 8H), 1.63-1.74 (m, 2H), 1.85-1.93 (m, 2H), 2.61 (m, 1H), 2.85-2.97 (m, 2H), 3.78-3.94 (m, 2H), 6.45 (d, J=9.00 Hz, 1H), 6.64 (m, 1H), 7.15 (d, J=9.76 Hz, 1H), 7.23 (d, J=7.63 Hz, 6H), 7.28-7.31 (m, 3H), 7.33-7.36 (m, 6H), 7.76 (d, J=8.39 Hz, 1H), 7.80-7.87 (m, 3H), 10.68 (br. s., 1H). HRMS (ESI) calcd for C50H49ClN5O5 [M+H]⁺ 834.3417, found 834.3402.

### 1-[(Tetrahydro-2H-pyran-4-ylcarbonyl)oxy]ethyl (3R)-3-{[5-(2-chloro-5-cyanophenyl)-1-trityl-1H-indazol-3-yl]carbamoyl}piperidine-1-carboxylate [(XIIIh), R1=2-chloro-5-cyanophenyl, R2=1-[(tetrahydro-2H-pyran-4-ylcarbonyl)oxy]ethyl piperidine-1-carboxylate, R9=1,1′,1″-methanetriyltribenzene]

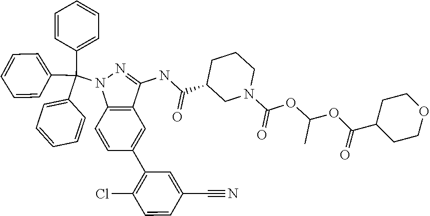 [SMILES: CC(OC(=O)C1CCOCC1)OC(=O)N1CCC[C@@H](C(=O)Nc2nn(C(c3ccccc3)(c3ccccc3)c3ccccc3)c3ccc(-c4cc(C#N)ccc4Cl)cc23)C1]

Starting from 1-chloroethyl (3R)-3-{[5-(2-chloro-5-cyanophenyl)-1-trityl-1H-indazol-3-yl]carbamoyl}piperidine-1-carboxylate and tetrahydro-pyran-4-carboxylic acid. Flash column chromatography (Hexane/EtOAc 50:50). Obtained 0.073 g (42% of yield). LCMS (ESI) m/z 822 (M+H)⁺.

### 1-[(2,2-Dimethylpropanoyl)oxy]propyl (3R)-3-{[5-(2-chloro-5-cyanophenyl)-1-trityl-1H-indazol-3-yl]carbamoyl}-piperidine-1-carboxylate [(XIIIh), R1=2-chloro-5-cyanophenyl, R2=1-[(2,2-dimethylpropanoyl)oxy]propyl piperidine-1-carboxylate, R9=1,1′,1″-methanetriyltribenzene]

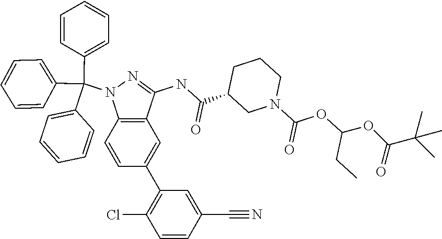 [SMILES: CCC(OC(=O)N1CCC[C@@H](C(=O)Nc2nn(C(c3ccccc3)(c3ccccc3)c3ccccc3)c3ccc(-c4cc(C#N)ccc4Cl)cc23)C1)OC(=O)C(C)(C)C]

Starting from 1-chloropropyl (3R)-3-{[5-(2-chloro-5-cyanophenyl)-1-trityl-1H-indazol-3-yl]carbamoyl}piperidine-1-carboxylate and pivalic acid. Flash column chromatography (Hexane/EtOAc 70:30). Obtained 0.055 g (71% of yield). LCMS (ESI) m/z 808 (M+H)⁺.

### Example 3

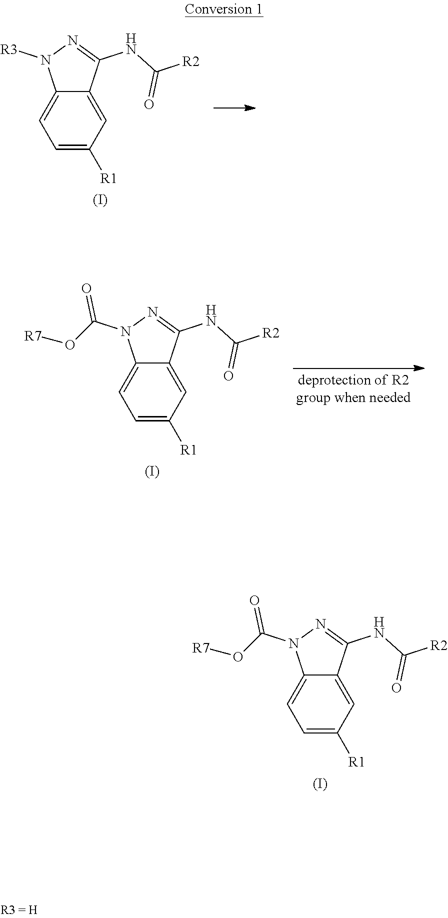

### tert-Butyl (3R)-3-{[5-(2-chloro-5-cyanophenyl)-1H-indazol-3-yl]carbamoyl}piperidine-1-carboxylate [(I), R1=2-chloro-5-cyanophenyl, R2=tert-butyl piperidine-1-carboxylate, R3=H]

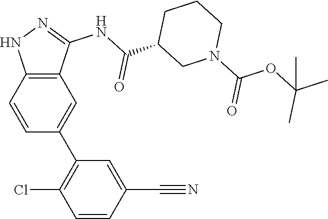 [SMILES: CC(C)(C)OC(=O)N1CCC[C@@H](C(=O)Nc2n[nH]c3ccc(-c4cc(C#N)ccc4Cl)cc23)C1]

To a mixture of (3R)-N-[5-(2-chloro-5-cyanophenyl)-1H-indazol-3-yl]piperidine-3-carboxamide hydrochloride (0.2 g, 0.48 mmol) and cesium carbonate (0.469 g, 1.44 mmol) in dry DMF (8 mL), cooled at 0-5° C., a solution of di-tert-butyl dicarbonate (0.094 g, 0.432 mmol) in dry THF (2 mL) was added dropwise. The reaction was stirred at 0-5° C. for 2 h, then was diluted with EtOAc (20 mL) and washed with water (40 mL) and brine (20 mL). The organic phase was separated, dried with anhydrous Na₂SO₄, filtered and evaporated. The crude was purified by flash column chromatography (DCM/MeOH 95:5) to give tert-butyl (3R)-3-{[5-(2-chloro-5-cyanophenyl)-1H-indazol-3-yl]carbamoyl}piperidine-1-carboxylate (0.204 g, 89% of yield) as white solid. ¹H NMR (500 MHz, DMSO-d₆) δ ppm 1.34-1.41 (m, 10H), 1.58-1.72 (m, 2H), 1.97-1.99 (m, 1H), 2.59 (m, 1H), 2.74-2.84 (m, 2H), 3.82 (d, J=12.66 Hz, 1H), 3.90-4.11 (m, 1H), 7.42 (dd, J=8.54, 1.37 Hz, 1H), 7.54 (d, J=8.69 Hz, 1H), 7.80 (d, J=8.39 Hz, 1H), 7.85 (s, 1H), 7.87 (dd, J=8.24, 1.98 Hz, 1H), 7.92 (d, J=1.83 Hz, 1H), 10.53 (s, 1H), 12.86 (s, 1H). HRMS (ESI) calcd for C25H27ClN5O3 [M+H]⁺ 480.1797, found 480.1779.

### tert-Butyl 3-({[(3+)-1-(tert-butoxycarbonyl)-6,6-dimethylpiperidin-3-yl]carbonyl}amino)-5-(2-chloro-5-cyanophenyl)-1H-indazole-1-carboxylate [(I), R1=2-chloro-5-cyanophenyl, R2=tert-butyl 2,2-dimethylpiperidine-1-carboxylate, R7=tert-butyl]

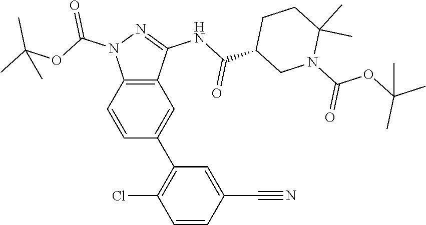 [SMILES: CC(C)(C)OC(=O)N1C[C@H](C(=O)Nc2nn(C(=O)OC(C)(C)C)c3ccc(-c4cc(C#N)ccc4Cl)cc23)CCC1(C)C]

To a solution of (+)-N-[5-(2-chloro-5-cyanophenyl)-1H-indazol-3-yl]-6,6-dimethylpiperidine-3-carboxamide hydrochloride (0.93 g, 2.093 mmol) and 4-(dimethylamino)pyridine (0.038 g, 0.314 mmol) in dichloromethane (28 mL) triethylamine (1.45 mL, 10.46 mmol) was added. To this solution was slowly added a solution of di-tert-butyl dicarbonate (1.142 g, 5.232 mmol) in DCM (6.0 mL). After 24 hours stirring at room temperature a further addition of di-tert-butyl dicarbonate (0.228 g, 1.047 mmol) in dichloromethane (1.5 mL) was done and the reaction mixture was stirred at room temperature for 24 hours. Then, the reaction mixture was washed with 5% citric acid solution (45 mL) and the aqueous layer was back extracted with dichloromethane (2×25 mL). The combined extracts were washed with brine (30 mL), dried with anhydrous sodium sulfate and concentrated under reduced pressure. This material was purified by flash chromatography (Hexane/EtOAc 70:30) to afford tert-butyl 3-({[(3+)-1-(tert-butoxycarbonyl)-6,6-dimethylpiperidin-3-yl]carbonyl}amino)-5-(2-chloro-5-cyanophenyl)-1H-indazole-1-carboxylate (0.942 g, 74% of yield) as white solid. Absolute stereochemistry was not determined. 1H NMR (600 MHz, DMSO-d₆) δ ppm 1.31 (s, 9H), 1.32 (s, 3H), 1.39 (s, 3H), 1.54-1.59 (m, 1H), 1.63-1.65 (m, 10H), 1.79-1.90 (m, 2H), 2.85 (m, 1H), 3.41 (dd, J=13.53, 8.45 Hz, 1H), 3.80 (dd, J=13.62, 5.09 Hz, 1H), 7.70 (dd, J=8.72, 1.63 Hz, 1H), 7.84 (d, J=8.36 Hz, 1H), 7.92 (dd, J=8.36, 2.00 Hz, 1H), 7.96 (d, J=2.00 Hz, 1H), 8.03 (s, 1H), 8.17 (d, J=8.72 Hz, 1H), 11.05 (br. s., 1H). HRMS (ESI) calcd for C32H39ClN5O5 [M+H]⁺ 608.2634, found 608.2627.

### tert-Butyl (5+)-5-{[5-(2-chloro-5-cyanophenyl)-1H-indazol-3-yl]carbamoyl}-2,2-dimethylpiperidine-1-carboxylate [(I), R1=2-chloro-5-cyanophenyl, R2=tert-butyl 2,2-dimethylpiperidine-1-carboxylate, R3=H]

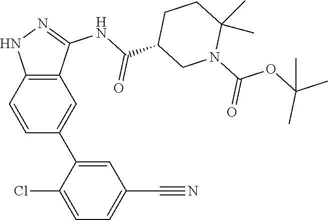 [SMILES: CC(C)(C)OC(=O)N1C[C@H](C(=O)Nc2n[nH]c3ccc(-c4cc(C#N)ccc4Cl)cc23)CCC1(C)C]

A solution of tert-butyl 3-({[(3+)-1-(tert-butoxycarbonyl)-6,6-dimethylpiperidin-3-yl]carbonyl}amino)-5-(2-chloro-5-cyanophenyl)-1H-indazole-1-carboxylate (0.935 g, 1.538 mmol) in 1,4-dioxane (25 mL) was treated with 2M sodium hydroxide (2.3 mL, 4.613 mmol) and heated to 80° C. for 3 h. The reaction mixture was concentrated up to ⅕ of initial volume and then diluted with EtOAc (25 mL) and washed with 5% aqueous citric acid (25 mL). The aqueous layer was back extracted with EtOAc (15 mL). The combined organic extracts were washed with brine (30 mL), dried with anhydrous sodium sulfate and concentrated. The crude was triturated with a mixture of tert-butylmethylether (7.5 mL) and Hexane (3.5 mL) at room temperature, aged at +5° C. for 2 h, filtered and finally dried in vacuum at +40° C. tert-Butyl (5+)-5-{[5-(2-chloro-5-cyanophenyl)-1H-indazol-3-yl]carbamoyl}-2,2-dimethylpiperidine-1-carboxylate (0.65 g, 83% of yield) was obtained as pink solid. yield. Absolute stereochemistry was not determined. 1H NMR (500 MHz, DMSO-d₆) δ ppm 1.31 (s, 3H), 1.33 (s, 9H), 1.40 (s, 3H), 1.52-1.57 (m, 1H), 1.63-1.69 (m, 1H), 1.76-1.90 (m, 2H), 2.79 (m, 1H), 3.83 (dd, J=13.57, 5.03 Hz, 1H), 7.41 (dd, J=8.77, 1.45 Hz, 1H), 7.52 (d, J=8.69 Hz, 1H), 7.80 (d, J=8.24 Hz, 1H), 7.85 (s, 1H), 7.87 (dd, J=8.24, 1.83 Hz, 1H), 7.91 (d, J=1.83 Hz, 1H), 10.49 (s, 1H), 12.84 (s, 1H). HRMS (ESI) calcd for C27H31ClN5O3 [M+H]⁺ 580.0118, found 580.0112.

### Butyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=butyl]

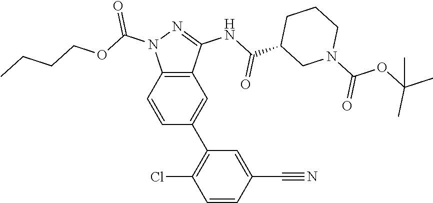 [SMILES: CCCCOC(=O)n1nc(NC(=O)[C@@H]2CCCN(C(=O)OC(C)(C)C)C2)c2cc(-c3cc(C#N)ccc3Cl)ccc21]

To a solution of tert-butyl (3R)-3-{[5-(2-chloro-5-cyanophenyl)-1H-indazol-3-yl]carbamoyl}piperidine-1-carboxylate (0.09 g, 0.188 mmol) and pyridine (0.046 mL, 0.564 mmol) in dry THF (3 mL), cooled at 0-5° C., a solution of butyl chloroformate (0.029 mL, 0.226 mmol) in dry THF (2 mL) was added dropwise. The reaction was stirred at 0-5° C. for 1 h, then was diluted with EtOAc (15 mL) and washed with water (10 mL) and brine (10 mL). The organic phase was separated, dried with anhydrous Na₂SO₄, filtered and evaporated. The crude was purified by flash column chromatography (Hexane/EtOAc 70:30) to give butyl 3-({[(3R)-1-(tert-butoxycarbonyl)piperidin-3-yl]carbonyl}amino)-5-(2-chloro-5-cyanophenyl)-1H-indazole-1-carboxylate (0.086 g, 79% of yield) as white solid. ¹H NMR (500 MHz, DMSO-d₆) δ ppm 0.95 (t, J=7.40 Hz, 3H), 1.30-1.39 (m, 10H), 1.45 (dq, J=14.98, 7.46 Hz, 2H), 1.64-1.83 (m, 4H), 1.97-2.00 (m, 1H), 2.63 (m, 1H), 2.81-2.95 (m, 2H), 3.79 (d, J=12.35 Hz, 1H), 3.94-4.06 (m, 1H), 4.46 (t, J=6.56 Hz, 2H), 7.73 (dd, J=8.69, 1.68 Hz, 1H), 7.85 (d, J=8.39 Hz, 1H), 7.93 (dd, J=8.39, 1.98 Hz, 1H), 7.99 (d, J=1.98 Hz, 1H), 8.10 (s, 1H), 8.20 (d, J=8.69 Hz, 1H), 11.12 (br. s., 1H). HRMS (ESI) calcd for C30H35ClN5O5 [M+H]⁺ 580.2321, found 580.2318.

According to this same methodology, by using the opportune intermediates and reagents, the following compounds were obtained:

### Ethyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=ethyl]

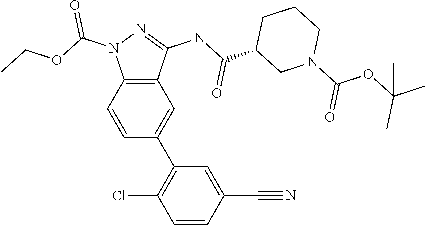 [SMILES: CCOC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and ethyl chloroformate. Flash column chromatography (Hexane/EtOAc 60:40). Obtained 0.083 g (63% of yield). ¹H NMR (500 MHz, DMSO-d₆) δ ppm 1.31-1.44 (m, 13H), 1.59-1.76 (m, 2H), 1.97-2.00 (m, 1H), 2.63 (m, 1H), 2.79-2.90 (m, 2H), 3.79 (d, J=13.42 Hz, 1H), 3.93-4.05 (m, 1H), 4.50 (q, J=7.12 Hz, 2H), 7.73 (dd, J=8.69, 1.68 Hz, 1H), 7.85 (d, J=8.39 Hz, 1H), 7.92 (dd, J=8.39, 1.98 Hz, 1H), 7.99 (d, J=2.14 Hz, 1H), 8.10 (s, 1H), 8.21 (d, J=8.85 Hz, 1H), 11.13 (br. s., 1H). HRMS (ESI) calcd for C28H31ClN5O5 [M+H]⁺ 552.2008, found 552.2001.

### Propan-2-yl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=propan-2-yl]

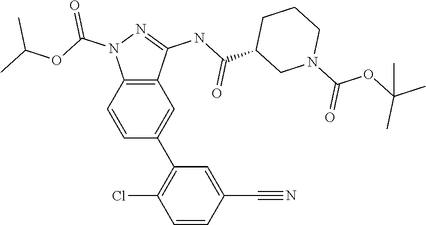 [SMILES: CC(C)OC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and isopropyl chloroformate. Flash column chromatography (Hexane/EtOAc 60:40). Obtained 0.076 g (81% of yield). ¹H NMR (500 MHz, DMSO-d₆) δ ppm 1.34-1.40 (m, 10H), 1.44 (d, J=6.25 Hz, 6H), 1.64-1.75 (m, 2H), 1.97-2.01 (m, 1H), 2.63 (m, 1H), 2.81-2.92 (m, 2H), 3.78 (d, J=14.18 Hz, 1H), 3.94-4.03 (m, 1H), 5.26 (spt, J=6.28 Hz, 1H), 7.73 (dd, J=8.77, 1.75 Hz, 1H), 7.86 (d, J=8.39 Hz, 1H), 7.93 (dd, J=8.39, 1.98 Hz, 1H), 8.00 (d, J=1.98 Hz, 1H), 8.11 (s, 1H), 8.23 (d, J=8.69 Hz, 1H), 11.16 (br. s., 1H). HRMS (ESI) calcd for C29H33ClN5O5 [M+H]⁺ 566.2165, found 566.2161.

### Hexyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=hexyl]

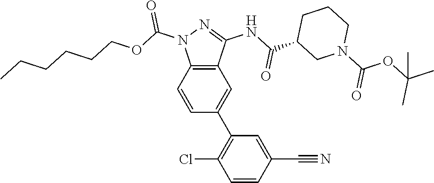 [SMILES: CCCCCCOC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and hexyl chloroformate. Flash column chromatography (Hexane/EtOAc 70:30). Obtained 0.088 g (87% of yield). ¹H NMR (500 MHz, DMSO-d₆) δ ppm 0.88 (t, J=7.17 Hz, 3H), 1.30-1.45 (m, 16H) 1.64-1.74 (m, 2H), 1.80 (m, 2H), 1.98-2.02 (m, 1H), 2.65 (m, 1H), 2.81-2.94 (m, 2H), 3.79 (d, J=12.20 Hz, 1H), 3.92-4.06 (m, 1H), 4.47 (t, J=6.63 Hz, 2H), 7.74 (dd, J=8.69, 1.68 Hz, 1H), 7.86 (d, J=8.39 Hz, 1H), 7.93 (dd, J=8.39, 2.14 Hz, 1H), 8.00 (d, J=1.98 Hz, 1H), 8.11 (s, 1H), 8.21 (d, J=8.85 Hz, 1H), 11.14 (br. s., 1H). HRMS (ESI) calcd for C32H39ClN5O5 [M+H]⁺ 608.2634, found 608.2629.

### 2,2-Dimethylpropyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=2,2-dimethylpropyl]

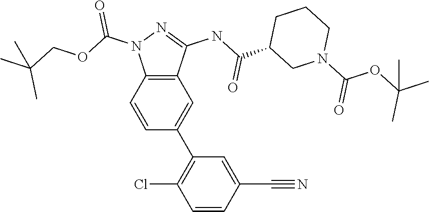 [SMILES: CC(C)(C)COC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and neopentyl chloroformate. Flash column chromatography (Hexane/EtOAc 70:30). Obtained 0.094 g (89% of yield). ¹H NMR (500 MHz, DMSO-d₆) δ ppm 1.04 (s, 9H), 1.32-1.41 (m, 10H), 1.63-1.74 (m, 2H), 1.97-2.01 (m, 1H), 2.63 (m, 1H), 2.81-2.90 (m, 2H), 3.77 (d, J=14.03 Hz, 1H), 3.94-4.05 (m, 1H), 4.21 (s, 2H), 7.75 (dd, J=8.69, 1.53 Hz, 1H), 7.85 (d, J=8.39 Hz, 1H), 7.92 (dd, J=8.39, 2.14 Hz, 1H), 7.99 (d, J=1.98 Hz, 1H), 8.07 (s, 1H), 8.20 (d, J=8.85 Hz, 1H), 11.09 (br. s., 1H).). HRMS (ESI) calcd for C31H37ClN5O5 [M+H]⁺ 594.2478, found 594.2468.

### 2-Methylpropyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=2-methylpropyl]

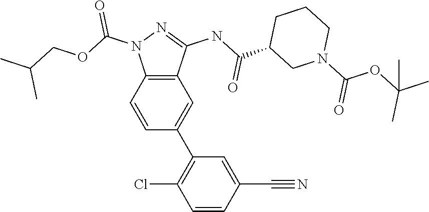 [SMILES: CC(C)COC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and isobutyl chloroformate. Flash column chromatography (Hexane/EtOAc 70:30). Obtained 0.156 g (86% of yield). ¹H NMR (500 MHz, DMSO-d₆) δ ppm 1.00 (d, J=6.71 Hz, 6H), 1.31-1.45 (m, 10H), 1.62-1.73 (m, 2H), 1.93-2.04 (m, 1H), 1.97-2.00 (m, 1H), 2.12 (m, 1H), 2.64 (m, 1H), 2.81-2.91 (m, 2H), 3.77 (d, J=12.51 Hz, 1H), 3.93-4.03 (m, 1H), 4.26 (d, J=6.71 Hz, 2H), 7.74 (dd, J=8.69, 1.53 Hz, 1H), 7.85 (d, J=8.39 Hz, 1H), 7.92 (dd, J=8.24, 1.98 Hz, 1H), 7.99 (d, J=1.98 Hz, 1H), 8.09 (s, 1H), 8.20 (d, J=8.69 Hz, 1H), 11.12 (br. s., 1H). HRMS (ESI) calcd for C30H35ClN5O5 [M+H]P 581.0745, found 581.0740.

### Pentyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=pentyl]

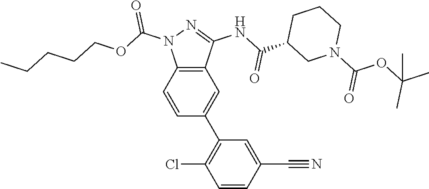 [SMILES: CCCCCOC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and pentyl chloroformate. Flash column chromatography (Hexane/EtOAc 60:40). Obtained 0.099 g (80% of yield). ¹H NMR (500 MHz, DMSO-d₆) δ ppm 0.90 (t, J=7.17 Hz, 3H), 1.31-1.44 (m, 14H), 1.62-1.73 (m, 2H), 1.79 (m, 2H), 1.75-1.83 (m, 2H), 1.97-2.00 (m, 1H), 2.64 (m, 1H), 2.80-2.92 (m, 2H), 3.78 (d, J=12.51 Hz, 1H), 3.92-4.05 (m, 1H), 4.45 (t, J=6.63 Hz, 2H), 7.73 (dd, J=8.77, 1.75 Hz, 1H), 7.85 (d, J=8.39 Hz, 1H), 7.92 (dd, J=8.39, 2.14 Hz, 1H), 7.99 (d, J=1.98 Hz, 1H), 8.10 (s, 1H), 8.20 (d, J=8.69 Hz, 1H), 11.12 (br. s., 1H). HRMS (ESI) calcd for C31H37ClN5O5 [M+H]⁺ 594.1010, found 594.1002.

### 2-Methoxyethyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=2-methoxyethyl]

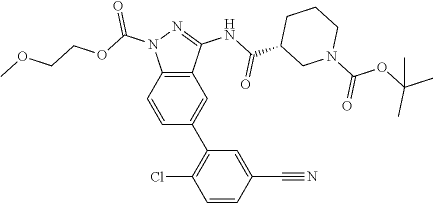 [SMILES: COCCOC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and 2-methoxyethyl chloroformate. Flash column chromatography (Hexane/EtOAc 60:40). Obtained 0.110 g (89% of yield). ¹H NMR (500 MHz, DMSO-d₆) δ ppm 1.31-1.40 (m, 10H), 1.62-1.73 (m, 2H), 1.97-2.01 (m, 1H), 2.64 (m, 1H), 2.80-2.90 (m, 2H), 3.32 (s, 3H), 3.72 (t, J=4.73 Hz, 2H), 3.78 (d, J=12.81 Hz, 1H), 3.92-4.06 (m, 1H), 4.58 (t, J=4.73 Hz, 2H), 7.73 (dd, J=8.77, 1.75 Hz, 1H), 7.85 (d, J=8.39 Hz, 1H), 7.92 (dd, J=8.39, 1.98 Hz, 1H), 8.00 (d, J=1.98 Hz, 1H), 8.11 (s, 1H), 8.21 (d, J=8.69 Hz, 1H), 11.15 (br. s., 1H). HRMS (ESI) calcd for C29H33ClN5O6 [M+H]⁺ 582.0473, found 582.0465.

### Cyclobutyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=cyclobutyl]

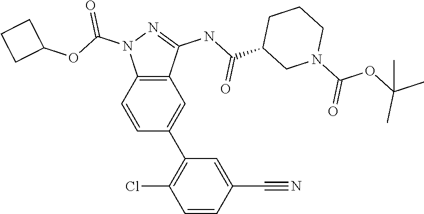 [SMILES: CC(C)(C)OC(=O)N1CCC[C@@H](C(=O)Nc2nn(C(=O)OC3CCC3)c3ccc(-c4cc(C#N)ccc4Cl)cc23)C1]

Starting from tert-butyl (3R)-3-{[5-(2-chloro-5-cyanophenyl)-1H-indazol-3-yl]carbamoyl}piperidine-1-carboxylate and cyclobutyl chloroformate. Flash column chromatography (Hexane/EtOAc 70:30). Obtained 0.077 g (91% of yield). ¹H NMR (500 MHz, DMSO-d₆) δ ppm 1.32-1.43 (m, 10H), 1.63-1.73 (m, 3H), 1.86 (m, 1H), 1.97-2.00 (m, 1H), 2.26 (m, 2H), 2.45 (m, 2H), 2.63 (m, 1H), 2.79-2.95 (m, 2H), 3.78 (d, J=12.96 Hz, 1H), 3.93-4.07 (m, 1H), 5.26 (m, 1H), 7.72 (dd, J=8.77, 1.75 Hz, 1H), 7.85 (d, J=8.24 Hz, 1H), 7.93 (dd, J=8.39, 1.98 Hz, 1H), 7.99 (d, J=1.98 Hz, 1H), 8.11 (s, 1H), 8.21 (d, J=8.69 Hz, 1H), 11.15 (br. s., 1H). HRMS (ESI) calcd for C30H33ClN5O5 [M+H]⁺ 578.0586, found 578.0591.

### Pentan-3-yl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=pentan-3-yl]

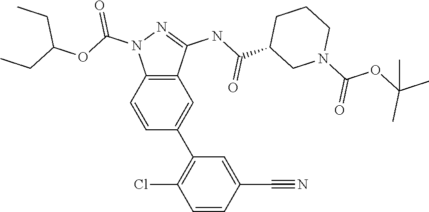 [SMILES: CCC(CC)OC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and pentan-3-yl chloroformate. Flash column chromatography (Hexane/EtOAc 60:40). Obtained 0.066 g (81% of yield). LCMS (ESI) m/z 594 (M+H)⁺.

### Cyclohexylmethyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=cyclohexylmethyl]

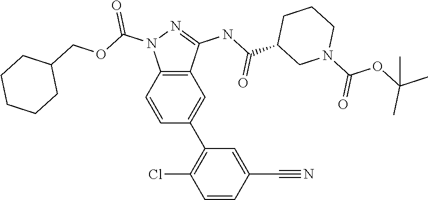 [SMILES: CC(C)(C)OC(=O)N1CCC[C@@H](C(=O)Nc2nn(C(=O)OCC3CCCCC3)c3ccc(-c4cc(C#N)ccc4Cl)cc23)C1]

Starting from tert-butyl (3R)-3-{[5-(2-chloro-5-cyanophenyl)-1H-indazol-3-yl]carbamoyl}piperidine-1-carboxylate and cyclohexylmethyl chloroformate. Flash column chromatography (Hexane/EtOAc 70:30). Obtained 0.091 g (88% of yield). ¹H NMR (500 MHz, DMSO-d₆) δ ppm 1.03-1.28 (m, 6H), 1.33-1.40 (m, 10H), 1.63-1.81 (m, 7H), 1.97-2.00 (m, 1H), 2.63 (m, 1H), 2.81-2.94 (m, 2H), 3.77 (d, J=13.27 Hz, 1H), 3.89-4.05 (m, 1H), 4.29 (d, J=6.41 Hz, 2H), 7.73 (dd, J=8.69, 1.68 Hz, 1H), 7.85 (d, J=8.39 Hz, 1H), 7.92 (dd, J=8.39, 2.14 Hz, 1H), 7.99 (d, J=1.98 Hz, 1H), 8.10 (br. s., 1H), 8.20 (d, J=8.54 Hz, 1H), 11.12 (br. s., 1H). HRMS (ESI) calcd for C33H39ClN5O5 [M+H]⁺620.1383, found 620.1371.

### 4-Methylpentyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=4-methylpentyl]

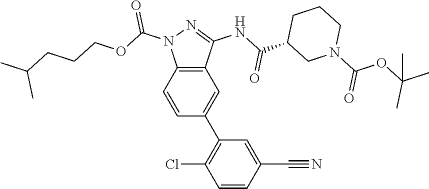 [SMILES: CC(C)CCCOC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and 4-methylpentyl chloroformate. Flash column chromatography (Hexane/EtOAc 70:30). Obtained 0.065 g (82% of yield). LCMS (ESI) m/z 608 (M+H)⁺.

### Cyclopropylmethyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=cyclopropylmethyl]

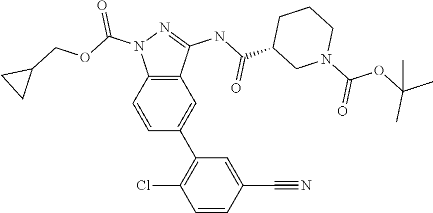 [SMILES: CC(C)(C)OC(=O)N1CCC[C@@H](C(=O)Nc2nn(C(=O)OCC3CC3)c3ccc(-c4cc(C#N)ccc4Cl)cc23)C1]

Starting from tert-butyl (3R)-3-{[5-(2-chloro-5-cyanophenyl)-1H-indazol-3-yl]carbamoyl}piperidine-1-carboxylate and cyclopropylmethyl chloroformate. Flash column chromatography (Hexane/EtOAc 70:30). Obtained 0.045 g (76% of yield). LCMS (ESI) m/z 578 (M+H)⁺.

### 2-(Ethylsulfanyl)ethyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=2-(ethylsulfanyl)ethyl]

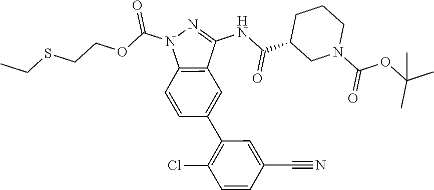 [SMILES: CCSCCOC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and 2-(ethylsulfanyl)ethyl chloroformate. Flash column chromatography (Hexane/EtOAc 80:20). Obtained 0.038 g (79% of yield). LCMS (ESI) m/z 612 (M+H)⁺.

### 3-(3-Methoxypropoxy)propyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=3-(3-methoxypropoxy)propyl]

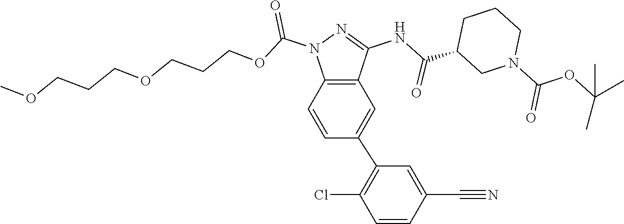 [SMILES: COCCCOCCCOC(=O)n1nc(NC(=O)[C@@H]2CCCN(C(=O)OC(C)(C)C)C2)c2cc(-c3cc(C#N)ccc3Cl)ccc21]

Starting from tert-butyl (3R)-3-{[5-(2-chloro-5-cyanophenyl)-1H-indazol-3-yl]carbamoyl}piperidine-1-carboxylate and 3-(3-methoxypropoxy)propyl chloroformate. Flash column chromatography (Hexane/EtOAc 50:50). Obtained 0.027 g (61% of yield). LCMS (ESI) m/z 654 (M+H)⁺.

### Cyclopentyl 3-({[(3R)-1-(tert-butoxycarbonyl)piperidin-3-yl]carbonyl}amino)-5-(2-chloro-5-cyanophenyl)-1H-indazole-1-carboxylate [(I), R1=2-chloro-5-cyanophenyl, R2=tert-butyl piperidine-1-carboxylate, R7=cyclopentyl]

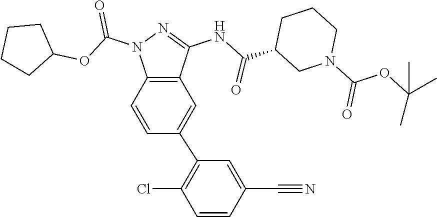 [SMILES: CC(C)(C)OC(=O)N1CCC[C@@H](C(=O)Nc2nn(C(=O)OC3CCCC3)c3ccc(-c4cc(C#N)ccc4Cl)cc23)C1]

Starting from tert-butyl (3R)-3-{[5-(2-chloro-5-cyanophenyl)-1H-indazol-3-yl]carbamoyl}piperidine-1-carboxylate and cyclopentyl chloroformate. Flash column chromatography (Hexane/EtOAc 70:30). Obtained 0.041 g (83% of yield). LCMS (ESI) m/z 592 (M+H)⁺.

### 2,2-Dimethylpropyl 3-({[(3+)-1-(tert-butoxycarbonyl)-6,6-dimethylpiperidin-3-yl]carbonyl}amino)-5-(2-chloro-5-cyanophenyl)-1H-indazole-1-carboxylate [(I), R1=2-chloro-5-cyanophenyl, R2=tert-butyl 2,2-dimethylpiperidine-1-carboxylate, R7=2,2-dimethylpropyl]

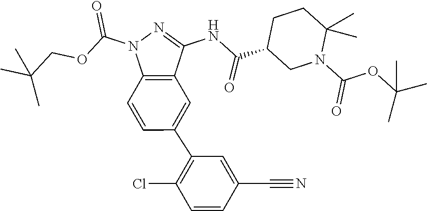 [SMILES: CC(C)(C)COC(=O)n1nc(NC(=O)[C@@H]2CCC(C)(C)N(C(=O)OC(C)(C)C)C2)c2cc(-c3cc(C#N)ccc3Cl)ccc21]

Starting from tert-butyl (5+)-5-{[5-(2-chloro-5-cyanophenyl)-1H-indazol-3-yl]carbamoyl}-2,2-dimethylpiperidine-1-carboxylate and neopentyl chloroformate. Flash column chromatography (Hexane/EtOAc 75:25). Obtained 0.121 g (66% of yield). Absolute stereochemistry was not determined. ¹H NMR (500 MHz, DMSO-d₆) δ ppm 1.04 (s, 9H), 1.30 (s, 9H), 1.33 (s, 3H), 1.38 (s, 3H), 1.50-1.73 (m, 2H), 1.76-1.94 (m, 2H), 2.88 (br. s., 1H), 3.43 (dd, J=13.57, 8.39 Hz, 1H), 3.80 (dd, J=13.65, 4.96 Hz, 1H), 4.21 (s, 2H), 7.74 (dd, J=8.77, 1.60 Hz, 1H), 7.85 (d, J=8.24 Hz, 1H), 7.92 (dd, J=8.39, 2.14 Hz, 1H), 7.97 (d, J=1.98 Hz, 1H), 8.05 (s, 1H), 8.20 (d, J=8.69 Hz, 1H), 11.06 (br. s., 1H). HRMS (ESI) calcd for C33H41ClN5O5 [M+H]P 621.1542, found 621.1535.

### Propan-2-yl 3-({[(3+)-1-(tert-butoxycarbonyl)-6,6-dimethylpiperidin-3-yl]carbonyl}amino)-5-(2-chloro-5-cyanophenyl)-1H-indazole-1-carboxylate [(I), R1=2-chloro-5-cyanophenyl, R2=tert-butyl 2,2-dimethylpiperidine-1-carboxylate, R7=propan-2-yl]

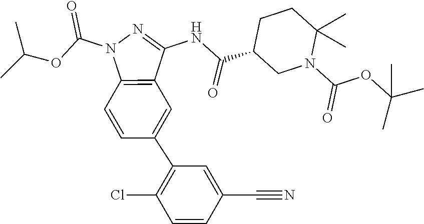 [SMILES: CC(C)OC(=O)n1nc(NC(=O)[C@@H]2CCC(C)(C)N(C(=O)OC(C)(C)C)C2)c2cc(-c3cc(C#N)ccc3Cl)ccc21]

Starting from tert-butyl (5+)-5-{[5-(2-chloro-5-cyanophenyl)-1H-indazol-3-yl]carbamoyl}-2,2-dimethylpiperidine-1-carboxylate and isopropyl chloroformate. Flash column chromatography (Hexane/EtOAc 70:30). Obtained 0.075 g (79% of yield). Absolute stereochemistry was not determined. ¹H NMR (600 MHz, DMSO-d₆) δ ppm 1.31 (s, 9H), 1.32 (s, 3H), 1.38 (s, 3H), 1.42 (d, J=6.36 Hz, 6H), 1.52-1.68 (m, 2H), 1.78-1.93 (m, 2H), 2.84 (br. s., 1H), 3.37-3.45 (m, 1H), 3.80 (dd, J=13.62, 5.09 Hz, 1H), 5.25 (spt, J=6.24 Hz, 1H), 7.72 (dd, J=8.72, 1.82 Hz, 1H), 7.85 (dd, J=8.36, 0.36 Hz, 1H), 7.92 (dd, J=8.36, 2.18 Hz, 1H), 7.96 (d, J=1.82 Hz, 1H), 8.08 (s, 1H), 8.21 (d, J=8.72 Hz, 1H), 11.11 (br. s., 1H). HRMS (ESI) calcd for C31H37ClN5O5 [M+H]⁺ 594.1010, found 594.1020.

### Hexyl 3-({[(3+)-1-(tert-butoxycarbonyl)-6,6-dimethylpiperidin-3-yl]carbonyl}amino)-5-(2-chloro-5-cyanophenyl)-1H-indazole-1-carboxylate [(I), R1=2-chloro-5-cyanophenyl, R2=tert-butyl 2,2-dimethylpiperidine-1-carboxylate, R7=hexyl]

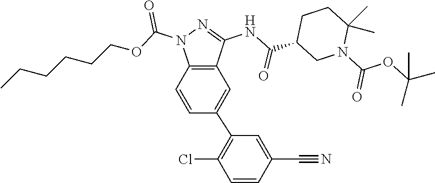 [SMILES: CCCCCCOC(=O)n1nc(NC(=O)[C@@H]2CCC(C)(C)N(C(=O)OC(C)(C)C)C2)c2cc(-c3cc(C#N)ccc3Cl)ccc21]

Starting from tert-butyl (5+)-5-{[5-(2-chloro-5-cyanophenyl)-1H-indazol-3-yl]carbamoyl}-2,2-dimethylpiperidine-1-carboxylate and hexyl chloroformate. Flash column chromatography (Hexane/EtOAc 75:25). Obtained 0.092 g (77% of yield). Absolute stereochemistry was not determined. ¹H NMR (500 MHz, DMSO-d₆) δ ppm 0.88 (t, J=7.02 Hz, 3H), 1.30-1.32 (m, 18H), 1.39 (s, 3H), 1.43 (t, J=7.40 Hz, 2H), 1.51-1.71 (m, 2H), 1.74-1.94 (m, 4H), 2.85 (br. s., 1H), 3.41 (dd, J=13.57, 8.39 Hz, 1H), 3.80 (dd, J=13.65, 4.96 Hz, 1H), 4.45 (t, J=6.63 Hz, 2H), 7.72 (dd, J=8.85, 1.68 Hz, 1H), 7.85 (d, J=8.39 Hz, 1H), 7.92 (dd, J=8.39, 1.98 Hz, 1H), 7.97 (d, J=1.98 Hz, 1H), 8.08 (s, 1H), 8.20 (d, J=8.69 Hz, 1H), 11.10 (br. s., 1H). HRMS (ESI) calcd for C34H43ClN5O5 [M+H]⁺ 636.1808, found 636.1815.

### Butyl 5-(2-chloro-5-cyanophenyl)-3-{[(3R)-piperidin-3-ylcarbonyl]amino}-1H-indazole-1-carboxylate hydrochloride (cpd 208) [(I), R1=2-chloro-5-cyanophenyl, R2=piperidine, R7=butyl]

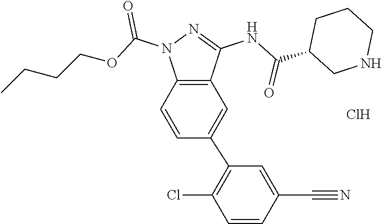 [SMILES: CCCCOC(=O)n1nc(NC(=O)[C@@H]2CCCNC2)c2cc(-c3cc(C#N)ccc3Cl)ccc21 ; Cl]

To a solution of butyl 3-({[(3R)-1-(tert-butoxycarbonyl)piperidin-3-yl]carbonyl}amino)-5-(2-chloro-5-cyanophenyl)-1H-indazole-1-carboxylate (0.080 g, 0.138 mmol) in dry DCM (5 mL), 4M HCl in dioxane (0.69 mL, 2.76 mmol) was added. The mixture was stirred at room temperature for 4 h, then the resulting solution was evaporated to dryness. The crude was treated with ethyl ether (10 mL), stirred for 10 min. and filtered. After drying at 40° C. under vacuo butyl 5-(2-chloro-5-cyanophenyl)-3-{[(3R)-piperidin-3-ylcarbonyl]amino}-1H-indazole-1-carboxylate hydrochloride (0.057 g, 80% of yield) was obtained as white solid. ¹H NMR (500 MHz, DMSO-d₆) δ ppm 0.96 (t, J=7.40 Hz, 3H), 1.46 (m, 2H), 1.64-1.74 (m, 2H), 1.74-1.81 (m, 2H), 1.81-1.88 (m, 1H), 2.10 (m, 1H), 2.90-3.03 (m, 2H), 3.07-3.22 (m, 2H), 4.47 (t, J=6.56 Hz, 2H), 7.75 (dd, J=8.69, 1.68 Hz, 1H), 7.86 (d, J=8.39 Hz, 1H), 7.93 (dd, J=8.24, 1.98 Hz, 1H), 7.98 (d, J=1.98 Hz, 1H), 8.10 (s, 1H), 8.21 (d, J=8.85 Hz, 1H), 8.64 (br. s., 2H), 11.32 (br. s., 1H). HRMS (ESI) calcd for C25H27ClN5O3 [M+H]⁺ 480.1797, found 480.18.

According to this same methodology, starting from the appropriate intermediates, the following compounds were obtained:

### Ethyl 5-(2-chloro-5-cyanophenyl)-3-{[(3R)-piperidin-3-ylcarbonyl]amino}-1H-indazole-1-carboxylate hydrochloride (cpd 209) [(I), R1=2-chloro-5-cyanophenyl, R2=piperidine, R7=ethyl]

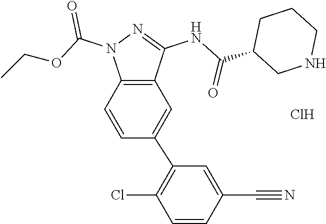 [SMILES: CCOC(=O)n1nc(NC(=O)[C@@H]2CCCNC2)c2cc(-c3cc(C#N)ccc3Cl)ccc21 ; Cl]

Obtained 0.055 g (79% of yield). ¹H NMR (500 MHz, DMSO-d₆) δ ppm 1.41 (t, J=7.09 Hz, 3H), 1.68-1.85 (m, 3H), 2.11 (m, J=8.54 Hz, 1H), 2.45 (m, 1H), 2.86-3.21 (m, 3H), 4.50 (q, J=7.17 Hz, 2H), 7.74 (dd, J=8.69, 1.68 Hz, 1H), 7.86 (d, J=8.24 Hz, 1H), 7.93 (dd, J=8.39, 2.14 Hz, 1H), 7.99 (d, J=2.13 Hz, 1H), 8.11 (s, 1H), 8.22 (d, J=8.69 Hz, 1H), 8.62 (br. s., 2H), 11.33 (br. s., 1H). HRMS (ESI) calcd for C23H23ClN5O3 [M+H]⁺ 452.1484, found 452.1478.

### Propan-2-yl 5-(2-chloro-5-cyanophenyl)-3-{[(3R)-piperidin-3-ylcarbonyl]amino}-1H-indazole-1-carboxylate hydrochloride (cpd 210) [(I), R1=2-chloro-5-cyanophenyl, R2=piperidine, R7=propan-2-yl]

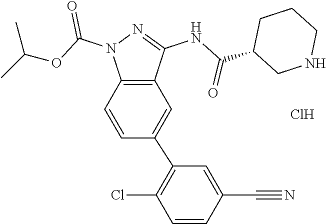 [SMILES: CC(C)OC(=O)n1nc(NC(=O)[C@@H]2CCCNC2)c2cc(-c3cc(C#N)ccc3Cl)ccc21 ; Cl]

Obtained 0.048 g (77% of yield). ¹H NMR (500 MHz, DMSO-d₆) δ ppm 1.44 (d, J=6.25 Hz, 6H), 1.68-1.85 (m, 3H), 2.11 (m, J=8.54 Hz, 1H), 2.90-3.21 (m, 4H), 5.26 (spt, J=6.25 Hz, 1H), 7.75 (dd, J=8.77, 1.75 Hz, 1H), 7.87 (d, J=8.39 Hz, 1H), 7.95 (dd, J=8.39, 2.14 Hz, 1H), 8.00 (d, J=1.98 Hz, 1H), 8.11 (s, 1H), 8.24 (d, J=8.85 Hz, 1H), 8.63 (br. s., 2H), 11.36 (br. s., 1H). HRMS (ESI) calcd for C24H25ClN5O3 [M+H]⁺ 466.1641, found 466.1627.

### Hexyl 5-(2-chloro-5-cyanophenyl)-3-{[(3R)-piperidin-3-ylcarbonyl]amino}-1H-indazole-1-carboxylate hydrochloride (cpd 211) [(I), R1=2-chloro-5-cyanophenyl, R2=piperidine, R7=hexyl]

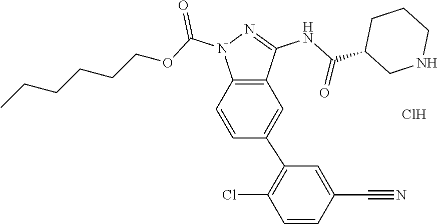 [SMILES: CCCCCCOC(=O)n1nc(NC(=O)[C@@H]2CCCNC2)c2cc(-c3cc(C#N)ccc3Cl)ccc21 ; Cl]

Obtained 0.058 g (82% of yield). 1H NMR (500 MHz, DMSO-d₆) δ ppm 0.88 (t, J=7.02 Hz, 3H), 1.26-1.38 (m, 4H), 1.43 (m, 2H), 1.63-1.89 (m, 5H), 2.10 (m, 1H), 2.52 (m, 1H), 2.87-2.95 (m, 1H), 3.00 (m, 1H), 3.06-3.13 (m, 1H), 3.18 (m, J=12.81 Hz, 1H), 4.46 (t, J=6.63 Hz, 2H), 7.74 (dd, J=8.85, 1.68 Hz, 1H), 7.86 (d, J=8.39 Hz, 1H), 7.93 (dd, J=8.39, 2.14 Hz, 1H), 7.99 (d, J=1.83 Hz, 1H), 8.10 (s, 1H), 8.21 (d, J=8.69 Hz, 1H), 8.63 (br. s., 2H), 11.32 (br. s., 1H). HRMS (ESI) calcd for C27H31ClN5O3 [M+H]⁺ 508.2110, found 508.2106.

### 2,2-Dimethylpropyl 5-(2-chloro-5-cyanophenyl)-3-{[(3R)-piperidin-3-ylcarbonyl]amino}-1H-indazole-1-carboxylate hydrochloride (cpd 212) [(I), R1=2-chloro-5-cyanophenyl, R2=piperidine, R7=2,2-dimethylpropyl]

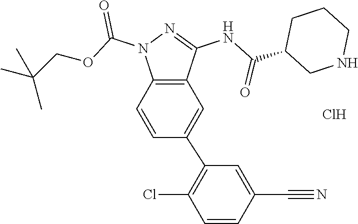 [SMILES: CC(C)(C)COC(=O)n1nc(NC(=O)[C@@H]2CCCNC2)c2cc(-c3cc(C#N)ccc3Cl)ccc21 ; Cl]

Obtained 0.064 g (80% of yield). ¹H NMR (500 MHz, DMSO-d₆) δ ppm 1.04 (s, 9H), 1.68-1.85 (m, 3H), 2.11 (m, 1H), 2.88-3.19 (m, 4H), 4.22 (s, 2H), 7.76 (dd, J=8.69, 1.68 Hz, 1H), 7.86 (d, J=8.39 Hz, 1H), 7.93 (dd, J=8.39, 1.98 Hz, 1H), 7.99 (d, J=1.98 Hz, 1H), 8.07 (s, 1H), 8.21 (d, J=8.69 Hz, 1H), 8.63 (br. s., 2H), 11.28 (br. s., 1H). HRMS (ESI) calcd for C26H29ClN5O3 [M+H]⁺ 494.1954, found 494.1930.

### 2-Methylpropyl 5-(2-chloro-5-cyanophenyl)-3-{[(3R)-piperidin-3-ylcarbonyl]amino}-1H-indazole-1-carboxylate hydrochloride (cpd 213) [(I), R1=2-chloro-5-cyanophenyl, R2=piperidine, R7=2-methylpropyl]

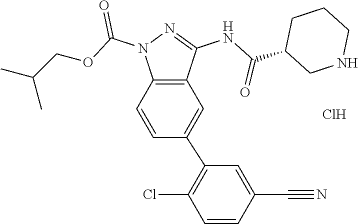 [SMILES: CC(C)COC(=O)n1nc(NC(=O)[C@@H]2CCCNC2)c2cc(-c3cc(C#N)ccc3Cl)ccc21 ; Cl]

Obtained 0.116 g (87% of yield). ¹H NMR (500 MHz, DMSO-d₆) δ ppm 1.01 (d, J=6.71 Hz, 6H), 1.58-1.78 (m, 2H), 1.78-1.90 (m, 1H), 2.09-2.15 (m, 2H), 2.83-2.95 (m, 1H), 3.01 (m, 1H), 3.10 (t, J=11.21 Hz, 1H), 3.17 (d, J=12.35 Hz, 1H), 4.27 (d, J=6.56 Hz, 2H), 7.75 (dd, J=8.77, 1.60 Hz, 1H), 7.86 (d, J=8.39 Hz, 1H), 7.93 (dd, J=8.39, 1.98 Hz, 1H), 7.99 (d, J=1.98 Hz, 1H), 8.09 (s, 1H), 8.21 (d, J=8.69 Hz, 1H), 8.65 (br. s., 2H), 11.32 (br. s., 1H). HRMS (ESI) calcd for C25H27ClN5O3 [M+H]⁺ 480.9586, found 480.9580.

### Pentyl 5-(2-chloro-5-cyanophenyl)-3-{[(3R)-piperidin-3-ylcarbonyl]amino}-1H-indazole-1-carboxylate hydrochloride (cpd 214) [(I), R1=2-chloro-5-cyanophenyl, R2=piperidine, R7=pentyl]

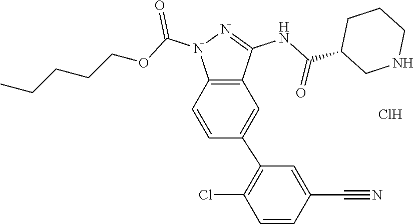 [SMILES: CCCCCOC(=O)n1nc(NC(=O)[C@@H]2CCCNC2)c2cc(-c3cc(C#N)ccc3Cl)ccc21 ; Cl]

Obtained 0.070 g (83% of yield). ¹H NMR (500 MHz, DMSO-d₆) δ ppm 0.90 (t, J=7.02 Hz, 3H), 1.31-1.47 (m, 4H), 1.63-1.91 (m, 5H), 2.11 (m, 1H), 2.83-3.23 (m, 4H), 4.46 (t, J=6.71 Hz, 2H), 7.75 (dd, J=8.77, 1.45 Hz, 1H), 7.86 (d, J=8.39 Hz, 1H), 7.93 (dd, J=8.39, 1.98 Hz, 1H), 7.99 (d, J=1.98 Hz, 1H), 8.10 (s, 1H), 8.21 (d, J=8.69 Hz, 1H), 8.65 (br. s., 2H), 11.32 (br. s., 1H). HRMS (ESI) calcd for C26H29ClN5O3 [M+H]⁺ 494.9852, found 494.9843.

### 2-Methoxyethyl 5-(2-chloro-5-cyanophenyl)-3-{[(3R)-piperidin-3-ylcarbonyl]amino}-1H-indazole-1-carboxylate hydrochloride (cpd 215) [(I), R1=2-chloro-5-cyanophenyl, R2=piperidine, R7=2-methoxyethyl]

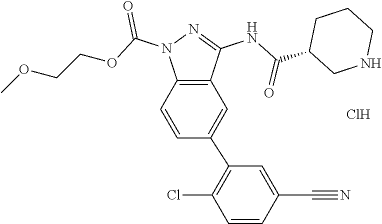 [SMILES: COCCOC(=O)n1nc(NC(=O)[C@@H]2CCCNC2)c2cc(-c3cc(C#N)ccc3Cl)ccc21 ; Cl]

Obtained 0.083 g (85% of yield). ¹H NMR (500 MHz, DMSO-d₆) δ ppm 1.66-1.86 (m, 3H), m (br. s., 1H), 2.89-3.19 (m, 4H), 3.33 (s, 3H), 3.72 (m, 2H), 4.59 (m, 2H), 7.75 (dd, J=8.77, 1.75 Hz, 1H), 7.85 (d, J=8.39 Hz, 1H), 7.93 (dd, J=8.39, 1.98 Hz, 1H), 7.99 (d, J=1.98 Hz, 1H), 8.11 (s, 1H), 8.22 (d, J=8.69 Hz, 1H), 8.66 (br. s., 2H), 11.35 (s, 1H). HRMS (ESI) calcd for C24H25ClN5O4 [M+H]⁺ 482.9315, found 482.9321.

### Cyclobutyl 5-(2-chloro-5-cyanophenyl)-3-{[(3R)-piperidin-3-ylcarbonyl]amino}-1H-indazole-1-carboxylate hydrochloride (cpd 216) [(I), R1=2-chloro-5-cyanophenyl, R2=piperidine, R7=cyclobutyl]

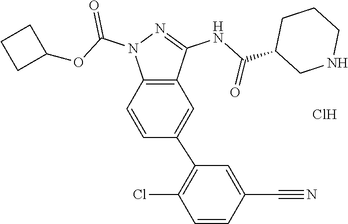 [SMILES: N#Cc1ccc(Cl)c(-c2ccc3c(c2)c(NC(=O)[C@@H]2CCCNC2)nn3C(=O)OC2CCC2)c1 ; Cl]

Obtained 0.031 g (84% of yield). ¹H NMR (500 MHz, DMSO-d₆) δ ppm 1.67-1.75 (m, 3H), 1.85 (m, 2H), 2.10 (m, 1H), 2.06-2.16 (m, 1H), 2.17-2.32 (m, 2H), 2.39-2.48 (m, 2H), 2.80-3.27 (m, 4H), 5.26 (quin, J=7.40 Hz, 1H), 7.74 (dd, J=8.77, 1.75 Hz, 1H), 7.85 (d, J=8.39 Hz, 1H), 7.93 (dd, J=8.39, 1.98 Hz, 1H), 7.99 (d, J=1.83 Hz, 1H), 8.11 (s, 1H), 8.22 (d, J=8.85 Hz, 1H), 8.67 (br. s., 2H), 11.35 (br. s., 1H). HRMS (ESI) calcd for C25H25ClN5O3 [M+H]⁺ 478.9428, found 478.9420.

### Pentan-3-yl 5-(2-chloro-5-cyanophenyl)-3-{[(3R)-piperidin-3-ylcarbonyl]amino}-1H-indazole-1-carboxylate hydrochloride (cpd 217) [(I), R1=2-chloro-5-cyanophenyl, R2=piperidine, R7=pentan-3-yl]

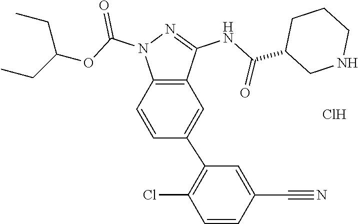 [SMILES: CCC(CC)OC(=O)n1nc(NC(=O)[C@@H]2CCCNC2)c2cc(-c3cc(C#N)ccc3Cl)ccc21 ; Cl]

Obtained 0.034 g (88% of yield). ¹H NMR (500 MHz, DMSO-d₆) δ ppm 0.95 (t, J=7.47 Hz, 6H), 1.68-1.84 (m, 7H), 2.11 (m, 1H), 2.88-3.19 (m, 4H), 5.01 (m, 1H), 7.74 (d, J=8.85 Hz, 1H), 7.85 (d, J=8.39 Hz, 1H) 7.94 (d, J=8.39 Hz, 1H), 7.98 (s, 1H), 8.10 (s, 1H), 8.22 (d, J=8.85 Hz, 1H), 8.66 (br. s., 2H), 11.36 (br. s., 1H). HRMS (ESI) calcd for C26H29ClN5O3 [M+H]⁺ 494.9852, found 494.9862.

### Cyclohexylmethyl 5-(2-chloro-5-cyanophenyl)-3-{[(3R)-piperidin-3-ylcarbonyl]amino}-1H-indazole-1-carboxylate hydrochloride (cpd 218) [(I), R1=2-chloro-5-cyanophenyl, R2=piperidine, R7=cyclohexylmethyl]

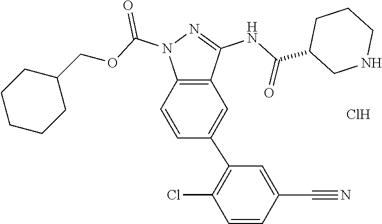 [SMILES: N#Cc1ccc(Cl)c(-c2ccc3c(c2)c(NC(=O)[C@@H]2CCCNC2)nn3C(=O)OCC2CCCCC2)c1 ; Cl]

Obtained 0.058 g (91% of yield). ¹H NMR (500 MHz, DMSO-d₆) δ ppm 1.03-1.30 (m, 6H), 1.60-1.85 (m, 8H), 2.11 (m, 1H), 2.89-3.18 (m, 4H), 4.29 (d, J=6.25 Hz, 2H), 7.75 (dd, J=8.77, 1.60 Hz, 1H), 7.86 (d, J=8.24 Hz, 1H), 7.93 (dd, J=8.24, 1.98 Hz, 1H), 7.99 (d, J=1.98 Hz, 1H), 8.09 (s, 1H), 8.20 (d, J=8.85 Hz, 1H), 8.65 (br. s., 2H), 11.32 (br. s., 1H). HRMS (ESI) calcd for C28H31ClN5O3 [M+H]⁺ 520.0225, found 520.0231.

### Cyclopropylmethyl 5-(2-chloro-5-cyanophenyl)-3-{[(3R)-piperidin-3-ylcarbonyl]amino}-1H-indazole-1-carboxylate formate (cpd 219) [(I), R1=2-chloro-5-cyanophenyl, R2=piperidine, R7=cyclopropylmethyl]

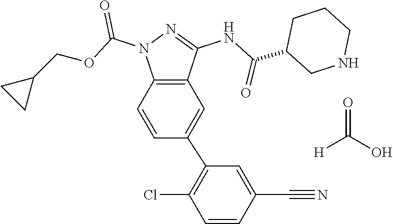

Purified by preparative HPLC. Obtained 0.020 g (72% of yield). ¹H NMR (500 MHz, DMSO-d₆) δ ppm 0.43 (m, 2H), 0.63 (m, 2H), 1.31 (m, 1H), 1.45-1.54 (m, 1H), 1.60-1.72 (m, 2H), 1.92-2.04 (m, 1H), 2.60-2.67 (m, 1H), 2.74 (m, 1H), 2.79-2.85 (m, 1H), 2.95 (d, J=12.35 Hz, 1H), 3.12-3.19 (m, 1H), 4.30 (d, J=7.47 Hz, 2H), 7.72 (dd, J=8.69, 1.68 Hz, 1H), 7.85 (d, J=8.39 Hz, 1H), 7.93 (dd, J=8.39, 1.98 Hz, 1H), 8.00 (d, J=1.98 Hz, 1H), 8.11 (d, J=0.92 Hz, 1H), 8.22 (d, J=8.54 Hz, 1H), 8.29 (s, 1H), 11.20 (br. s., 1H). HRMS (ESI) calcd for C25H25ClN5O3 [M+H]⁺ 478.9428, found 478.9425.

### 2-(Ethylsulfanyl)ethyl 5-(2-chloro-5-cyanophenyl)-3-{[(3R)-piperidin-3-ylcarbonyl]amino}-1H-indazole-1-carboxylate formate (cpd 220) [(I), R1=2-chloro-5-cyanophenyl, R2=piperidine, R7=2-(ethylsulfanyl)ethyl]

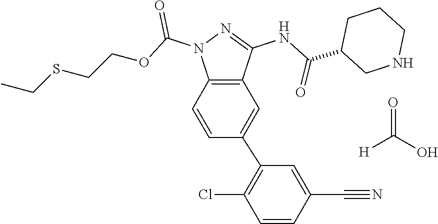

Purified by preparative HPLC. Obtained 0.018 g (75% of yield). ¹H NMR (500 MHz, DMSO-d₆) δ ppm 1.23 (t, J=7.40 Hz, 3H), 1.49-1.56 (m, 1H), 1.63-1.71 (m, 2H), 1.99-2.02 (m, 1H), 2.66 (m, 2H), 2.97 (t, J=6.71 Hz, 2H), 4.60 (t, J=6.71 Hz, 2H), 7.73 (dd, J=8.85, 1.83 Hz, 1H), 7.86 (d, J=8.39 Hz, 1H), 7.94 (dd, J=8.39, 1.98 Hz, 1H), 8.01 (d, J=1.98 Hz, 1H), 8.11 (d, J=1.07 Hz, 1H), 8.25 (d, J=8.69 Hz, 1H), 8.29 (s, 1H), 11.20 (br. s., 1H). HRMS (ESI) calcd for C25H27ClN5O3S [M+H]⁺ 512.0236, found 512.0245.

### 3-(3-Methoxypropoxy)propyl 5-(2-chloro-5-cyanophenyl)-3-{[(3R)-piperidin-3-ylcarbonyl]amino}-1H-indazole-1-carboxylate formate (cpd 221) [(I), R1=2-chloro-5-cyanophenyl, R2=piperidine, R7=3-(3-methoxypropoxy)propyl]

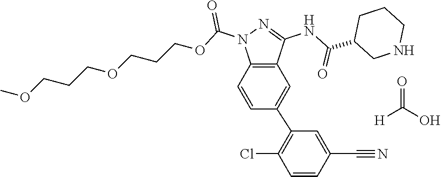

Purified by preparative HPLC. Obtained 0.015 g (58% of yield). 1H NMR (500 MHz, DMSO-d₆) δ ppm 0.99-1.07 (m, 2H), 1.37-1.39 (m, 2H), 1.51 (m, 1H), 1.61-1.71 (m, 2H), 1.99-2.01 (m, 1H), 2.63 (m, 1H), 2.71-2.84 (m, 2H), 2.93-2.99 (m, 1H), 3.68 (m, 4H), 5.26 (m, 2H), 7.72 (dd, J=8.85, 1.68 Hz, 1H), 7.85 (d, J=8.39 Hz, 1H), 7.93 (dd, J=8.39, 1.98 Hz, 1H), 8.00 (d, J=1.98 Hz, 1H), 8.09 (s, 1H), 8.21 (d, J=8.69 Hz, 1H), 8.28 (s, 1H), 11.19 (br. s., 1H). HRMS (ESI) calcd for C28H33ClN5O5 [M+H]⁺ 554.0372, found 554.0379.

### 4-Methylpentyl 5-(2-chloro-5-cyanophenyl)-3-{[(3R)-piperidin-3-ylcarbonyl]amino}-1H-indazole-1-carboxylate formate (cpd 222) [(I), R1=2-chloro-5-cyanophenyl, R2=piperidine, R7=4-methylpentyl]

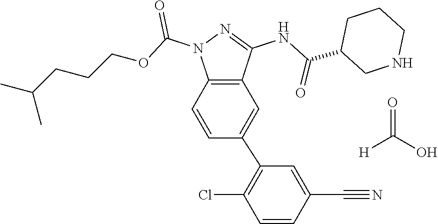

Purified by preparative HPLC. Obtained 0.023 g (62% of yield). ¹H NMR (500 MHz, DMSO-d₆) δ ppm 0.90 (d, J=6.71 Hz, 6H), 1.30 (m, 2H), 1.51-1.74 (m, 4H), 1.79 (m, 2H), 1.99-2.01 (m, 1H), 2.63-2.68 (m, 1H), 2.77 (m, 1H), 2.82-2.87 (m, 1H), 2.97-2.99 (m, 1H), 3.19 (d, J=9.61 Hz, 1H), 4.45 (t, J=6.71 Hz, 2H), 7.73 (dd, J=8.69, 1.68 Hz, 1H), 7.85 (d, J=8.24 Hz, 1H), 7.91 (dd, J=8.39, 1.98 Hz, 1H), 8.00 (d, J=1.98 Hz, 1H), 8.09 (s, 1H), 8.20 (d, J=8.69 Hz, 1H), 8.26 (s, 1H), 11.19 (br. s., 1H). HRMS (ESI) calcd for C27H31ClN5O3 [M+H]⁺ 508.0118, found 508.0112.

### Cyclopentyl 5-(2-chloro-5-cyanophenyl)-3-{[(3R)-piperidin-3-ylcarbonyl]amino}-1H-indazole-1-carboxylate formate (cpd 223) [(I), R1=2-chloro-5-cyanophenyl, R2=piperidine, R7=cyclopentyl]

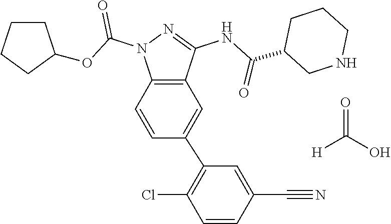

Purified by preparative HPLC. Obtained 0.029 g (69% of yield). ¹H NMR (500 MHz, DMSO-d₆) δ ppm 1.49-1.55 (m, 1H), 1.62-1.80 (m, 5H), 1.86-1.91 (m, 2H), 1.97-2.06 (m, 3H), 2.63-2.69 (m, 1H), 2.75-2.88 (m, 2H), 2.98-3.02 (m, 1H), 3.16-3.21 (m, 2H), 5.45 (tt, J=5.81, 2.80 Hz, 1H), 7.72 (dd, J=8.85, 1.68 Hz, 1H), 7.85 (d, J=8.39 Hz, 1H), 7.93 (dd, J=8.39, 1.98 Hz, 1H), 7.99 (d, J=1.98 Hz, 1H), 8.07 (s, 1H), 8.17 (d, J=8.85 Hz, 1H), 8.25 (s, 1H), 11.19 (br. s., 1H). HRMS (ESI) calcd for C26H27ClN5O3 [M+H]⁺ 492.9693, found 495.9689.

### 2,2-Dimethylpropyl 5-(2-chloro-5-cyanophenyl)-3-({[(3+)-6,6-dimethylpiperidin-3-yl]carbonyl}amino)-1H-indazole-1-carboxylate hydrochloride (cpd 224) [(I), R1=2-chloro-5-cyanophenyl, R2=tert-butyl 2,2-dimethylpiperidine-1-carboxylate, R7=2,2-dimethylpropyl]

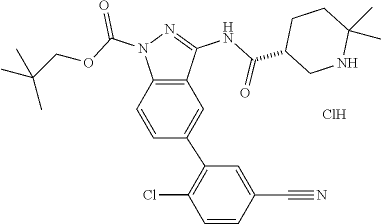 [SMILES: CC(C)(C)COC(=O)n1nc(NC(=O)[C@@H]2CCC(C)(C)NC2)c2cc(-c3cc(C#N)ccc3Cl)ccc21 ; Cl]

Obtained 0.081 g (75% of yield). Absolute stereochemistry was not determined. ¹H NMR (500 MHz, DMSO-d₆) δ ppm 1.05 (s, 9H), 1.30 (s, 3H), 1.34 (s, 3H), 1.61-1.85 (m, 3H), 2.05-2.09 (m, 1H), 2.98 (m, 1H), 3.11-3.23 (m, 1H), 4.22 (s, 2H), 7.76 (dd, J=8.77, 1.60 Hz, 1H), 7.86 (d, J=8.39 Hz, 1H), 7.94 (dd, J=8.39, 2.13 Hz, 1H), 7.99 (d, J=2.13 Hz, 1H), 8.09 (s, 1H), 8.21 (d, J=8.69 Hz, 1H), 8.85 (br. s., 2H), 11.29 (s, 1H). HRMS (ESI) calcd for C28H33ClN5O3 [M+H]⁺ 522.0384, found 522.0378.

### Hexyl 5-(2-chloro-5-cyanophenyl)-3-({[(3R)-6,6-dimethylpiperidin-3-yl]carbonyl}amino)-1H-indazole-1-carboxylate hydrochloride (cpd 225) [(I), R1=2-chloro-5-cyanophenyl, R2=tert-butyl 2,2-dimethylpiperidine-1-carboxylate, R7=hexyl]

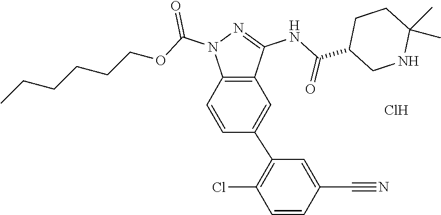 [SMILES: CCCCCCOC(=O)n1nc(NC(=O)[C@@H]2CCC(C)(C)NC2)c2cc(-c3cc(C#N)ccc3Cl)ccc21 ; Cl]

Obtained 0.055 g (70% of yield). Absolute stereochemistry was not determined. ¹H NMR (500 MHz, DMSO-d₆) δ ppm 0.88 (t, J=7.02 Hz, 3H), 1.29-1.34 (m, 10H), 1.43 (m, 2H), 1.61-1.83 (m, 5H), 2.02-2.08 (m, 1H), 2.92-2.99 (m, 1H), 3.14-3.19 (m, 1H), 3.32 (m, 1H), 4.46 (t, J=6.63 Hz, 2H), 7.74 (dd, J=8.69, 1.68 Hz, 1H), 7.86 (d, J=8.24 Hz, 1H), 7.94 (dd, J=8.39, 1.98 Hz, 1H), 7.98 (d, J=1.98 Hz, 1H), 8.12 (s, 1H), 8.21 (d, J=8.69 Hz, 1H), 8.84 (br. s., 2H), 11.33 (s, 1H). HRMS (ESI) calcd for C29H35ClN5O3 [M+H]⁺ 536.0650, found 536.0664.

### Propan-2-yl 5-(2-chloro-5-cyanophenyl)-3-({[(3R)-6,6-dimethylpiperidin-3-yl]carbonyl}amino)-1H-indazole-1-carboxylate hydrochloride (cpd 226) [(I), R1=2-chloro-5-cyanophenyl, R2=tert-butyl 2,2-dimethylpiperidine-1-carboxylate, R7=propan-2-yl]

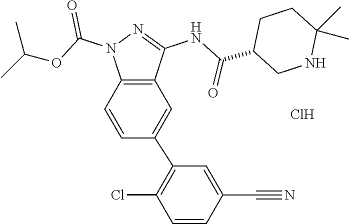 [SMILES: CC(C)OC(=O)n1nc(NC(=O)[C@@H]2CCC(C)(C)NC2)c2cc(-c3cc(C#N)ccc3Cl)ccc21 ; Cl]

Obtained 0.063 g (79% of yield). Absolute stereochemistry was not determined. 1H NMR (600 MHz, DMSO-d₆) δ ppm 1.29 (s, 3H), 1.34 (s, 3H), 1.43 (d, J=6.18 Hz, 6H), 1.61-1.67 (m, 1H), 1.72-1.76 (m, 1H), 1.78-1.84 (m, 1H), 2.02-2.08 (m, 1H), 2.91-2.97 (m, 1H), 3.15-3.19 (m, 1H), 3.37 (m, 1H), 5.25 (spt, J=6.27 Hz, 1H), 7.73 (dd, J=8.81, 1.73 Hz, 1H), 7.86 (dd, J=8.36, 0.36 Hz, 1H), 7.94 (dd, J=8.36, 2.18 Hz, 1H), 7.97 (d, J=2.18 Hz, 1H), 8.12 (s, 1H), 8.23 (d, J=8.72 Hz, 1H), 8.83 (br. s., 2H), 11.35 (br. s., 1H). HRMS (ESI) calcd for C26H29ClN5O3 [M+H]⁺ 494.9852, found 494.9842.

### Example 4

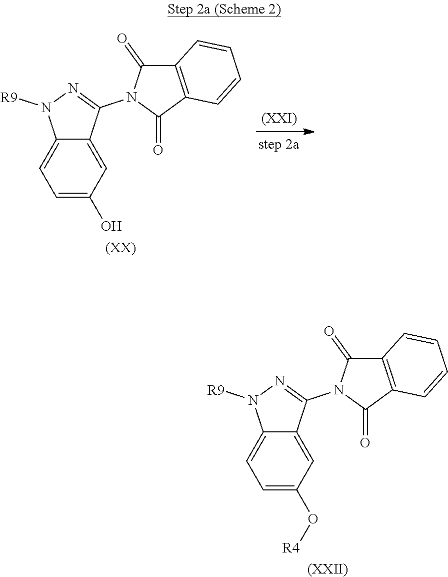

### 2-[5-(2-Fluoroethoxy)-1-trityl-1H-indazol-3-yl]-1H-isoindole-1,3(2H)-dione [(XXII), R4=fluoroethyl, R9=1,1′,1″-methanetriyltribenzene]

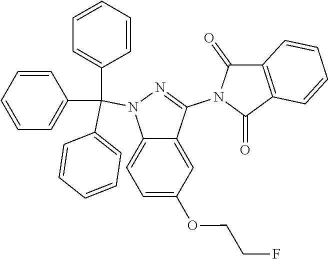 [SMILES: O=C1c2ccccc2C(=O)N1c1nn(C(c2ccccc2)(c2ccccc2)c2ccccc2)c2ccc(OCCF)cc12]

To a solution of 2-(5-hydroxy-1-trityl-1H-indazol-3-yl)-1H-isoindole-1,3(2H)-dione (0.953 g, 1.83 mmol) in dry DMF (15 mL), cesium carbonate (1.193 g, 3.66 mmol) and 1-fluoro-2-iodoethane (0.61 mL, 7.32 mmol) were added. The suspension was stirred at room temperature for 6 h, then diluted with EtOAc (20 mL) and washed with water (100 mL) and brine (50 mL). The organic phase was dried with anhydrous Na₂SO₄, filtered and evaporated to dryness. The white solid obtained (0.953 g, 95% of yield) was used for the next step without purification. ¹H NMR (500 MHz, DMSO-d₆) δ ppm 4.07-4.17 (m, 2H), 4.60-4.73 (m, 2H), 6.38 (d, J=9.30 Hz, 1H), 6.83 (dd, J=9.38, 2.52 Hz, 1H), 7.19-7.24 (m, 7H), 7.27-7.31 (m, 3H), 7.32-7.39 (m, 6H), 7.93-7.98 (m, 2H), 7.99-8.04 (m, 2H). HRMS (ESI) calcd for C36H27FN3O3Na [M+Na]⁺590.1850, found 590.1860.

According to this same methodology, using the appropriate reagents, the following intermediates were obtained:

### 2-[5-(2-Methoxyethoxy)-1-trityl-1H-indazol-3-yl]-1H-isoindole-1,3(2H)-dione [(XXII), R4=methoxyethyl, R9=1,1′,1″-methanetriyltribenzene]

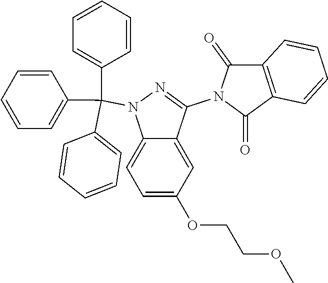 [SMILES: COCCOc1ccc2c(c1)c(N1C(=O)c3ccccc3C1=O)nn2C(c1ccccc1)(c1ccccc1)c1ccccc1]

Starting from 2-(5-hydroxy-1-trityl-1H-indazol-3-yl)-1H-isoindole-1,3(2H)-dione and 1-bromo-2-methoxyethane. Flash column chromatography (DCM/EtOAc 70:30). Obtained 0.150 g (70% of yield). ¹H NMR (500 MHz, DMSO-d₆) δ ppm 3.25 (s, 3H), 3.59 (dd, J=5.34, 3.81 Hz, 1H), 3.98 (dd, J=5.41, 3.89 Hz, 1H), 6.36 (d, J=9.30 Hz, 1H), 6.80 (dd, J=9.30, 2.44 Hz, 1H), 7.17-7.24 (m, 7H), 7.26-7.31 (m, 3H), 7.32-7.38 (m, 6H), 7.91-7.97 (m, 2H), 7.98-8.03 (m, 2H). HRMS (ESI) calcd for C37H30N3O4Na [M+Na]⁺602.2050, found 602.2056.

### 2-(5-Ethoxy-1-trityl-1H-indazol-3-yl)-1H-isoindole-1,3(2H)-dione [(XXII), R4=ethyl, R9=

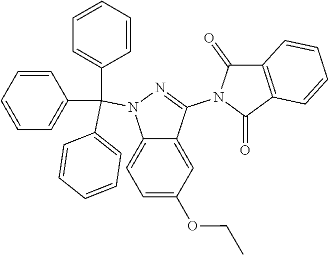 [SMILES: CCOc1ccc2c(c1)c(N1C(=O)c3ccccc3C1=O)nn2C(c1ccccc1)(c1ccccc1)c1ccccc1]

Starting from 2-(5-hydroxy-1-trityl-1H-indazol-3-yl)-1H-isoindole-1,3(2H)-dione and 1-bromoethane. Flash column chromatography (DCM/EtOAc 80:20). Obtained 0.115 g (75% of yield). LCMS (ESI) m/z 574 (M+Na)⁺.

### 2-[5-(Propan-2-yloxy)-1-trityl-1H-indazol-3-yl]-1H-isoindole-1,3(2H)-dione [(XXII), R4=2-propyl, R9=1,1′,1 methanetriyltribenzene]

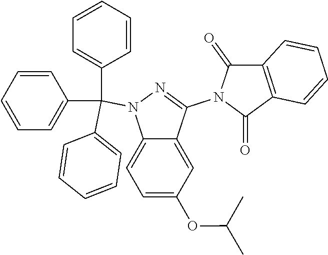 [SMILES: CC(C)Oc1ccc2c(c1)c(N1C(=O)c3ccccc3C1=O)nn2C(c1ccccc1)(c1ccccc1)c1ccccc1]

Starting from 2-(5-hydroxy-1-trityl-1H-indazol-3-yl)-1H-isoindole-1,3(2H)-dione and 2-iodopropane. Flash column chromatography (DCM/EtOAc 80:20). Obtained 0.210 g (78% of yield). LCMS (ESI) m/z 586 (M+Na)⁺.

### 2-(5-Butoxy-1-trityl-1H-indazol-3-yl)-1H-isoindole-1,3(2H)-dione [(XXII), R4=butyl, R9=methanetriyltribenzene]

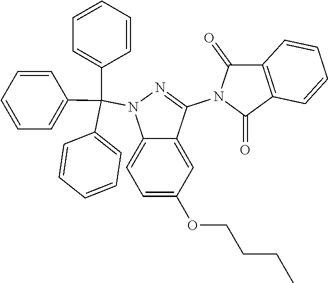 [SMILES: CCCCOc1ccc2c(c1)c(N1C(=O)c3ccccc3C1=O)nn2C(c1ccccc1)(c1ccccc1)c1ccccc1]

Starting from 2-(5-hydroxy-1-trityl-1H-indazol-3-yl)-1H-isoindole-1,3(2H)-dione and 1-bromobutane. Flash column chromatography (DCM/EtOAc 80:20). Obtained 0.312 g (81% of yield). LCMS (ESI) m/z 600 (M+Na)⁺.

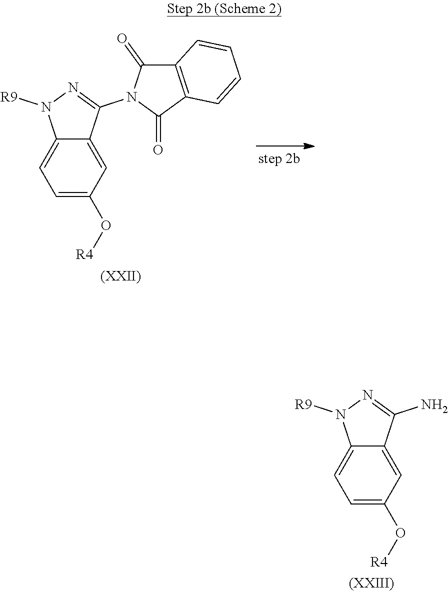

### 5-(2-Fluoroethoxy)-1-trityl-1H-indazol-3-amine [(XXIII), R4=fluoroethyl, R9=1,1′,1″-methanetriyltribenzene]

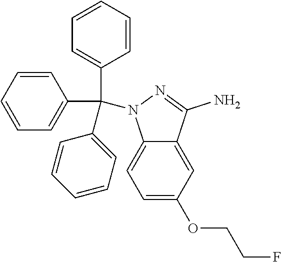 [SMILES: Nc1nn(C(c2ccccc2)(c2ccccc2)c2ccccc2)c2ccc(OCCF)cc12]

A solution of 2-[5-(2-fluoroethoxy)-1-trityl-1H-indazol-3-yl]-1H-isoindole-1,3(2H)-dione (0.983 g, 1.73 mmol) in 1M hydrazine in THE (17 mL, 17 mmol) was heated at reflux temperature for 1 h. After cooling at room temperature, the solid precipitated was filtered off washing with THE (10 mL) and the solution was evaporated to dryness. The crude obtained was dissolved in DCM (20 mL) and washed with 1M HCl (20 mL), aqueous NaHCO₃ (20 mL), water (20 mL) and brine (20 mL). The organic phase was separated, dried with anhydrous Na₂SO₄, filtered and evaporated to dryness.

5-(2-fluoroethoxy)-1-trityl-1H-indazol-3-amine (0.75 g, 99% of yield) was obtained as ivory solid and used for the next step without purification. 1H NMR (500 MHz, DMSO-d₆) δ ppm 4.08-4.14 (m, 2H), 4.65-4.74 (m, 2H), 5.5 (s, 2H), 6.24 (d, J=9.00 Hz, 1H), 6.67 (dd, J=9.15, 2.44 Hz, 1H), 7.15-7.21 (m, 7H), 7.25-7.28 (m, 3H), 7.33-7.36 (m, 6H). HRMS (ESI) calcd for C28H25FN3ONa [M+Na]⁺460.1795, found 460.1789.

According to this same methodology, starting from the appropriate intermediates, the following compounds were obtained:

### 5-(2-Methoxyethoxy)-1-trityl-1H-indazol-3-amine [(XXIII), R4=methoxyethyl, R9=1,1′,1″-methane-triyltribenzene]

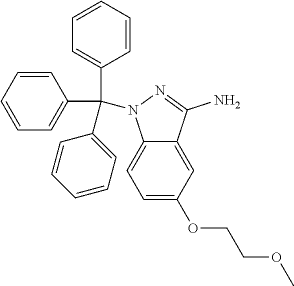 [SMILES: COCCOc1ccc2c(c1)c(N)nn2C(c1ccccc1)(c1ccccc1)c1ccccc1]

Starting from 2-[5-(2-methoxyethoxy)-1-trityl-1H-indazol-3-yl]-1H-isoindole-1,3(2H)-dione. Obtained 0.115 g (86% of yield). ¹H NMR (500 MHz, DMSO-d₆) δ ppm 3.28 (s, 3H), 3.59-3.65 (m, 2H), 3.98 (dd, J=5.41, 4.04 Hz, 2H), 5.48 (s, 2H), 6.24 (d, J=9.15 Hz, 1H), 6.65 (dd, J=9.15, 2.59 Hz, 1H), 7.14 (d, J=2.44 Hz, 1H), 7.17-7.22 (m, 3H), 7.26 (t, J=7.63 Hz, 6H), 7.31-7.37 (m, 6H).). HRMS (ESI) calcd for C29H28N3O2 [M+H]⁺ 450.2176, found 450.2164.

### 5-Ethoxy-1-trityl-1H-indazol-3-amine [(XXIII), R4=ethyl, R9=1,1′,1″-methanetriyltribenzene]

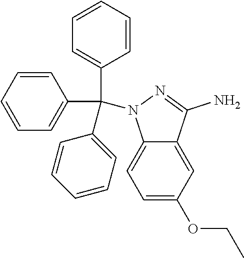 [SMILES: CCOc1ccc2c(c1)c(N)nn2C(c1ccccc1)(c1ccccc1)c1ccccc1]

Starting from 2-(5-ethoxy-1-trityl-1H-indazol-3-yl)-1H-isoindole-1,3(2H)-dione. Obtained 0.175 g (91% of yield). LCMS (ESI) m/z 420 (M+H)⁺.

### 5-(Propan-2-yloxy)-1-trityl-1H-indazol-3-amine [(XXIII), R4=2-propyl, R9=1,1′,1″-methanetriyltribenzene]

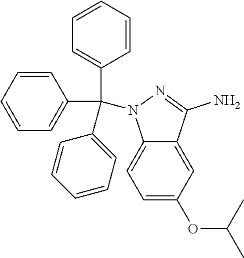 [SMILES: CC(C)Oc1ccc2c(c1)c(N)nn2C(c1ccccc1)(c1ccccc1)c1ccccc1]

Starting from 2-[5-(propan-2-yloxy)-1-trityl-1H-indazol-3-yl]-1H-isoindole-1,3(2H)-dione. Obtained 0.192 g (88% of yield). LCMS (ESI) m/z 434 (M+H)⁺.

### 5-Butoxy-1-trityl-1H-indazol-3-amine [(XXIII), R4=butyl, R9=1,1′,1″-methanetriyltribenzene]

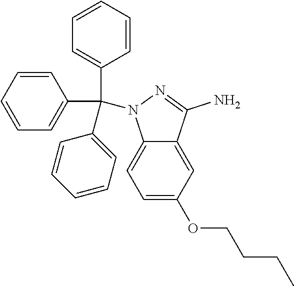 [SMILES: CCCCOc1ccc2c(c1)c(N)nn2C(c1ccccc1)(c1ccccc1)c1ccccc1]

Starting from 2-(5-butoxy-1-trityl-1H-indazol-3-yl)-1H-isoindole-1,3(2H)-dione. Obtained 0.175 g (91% of yield). LCMS (ESI) m/z 448 (M+H)⁺.

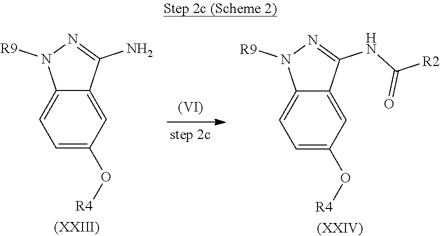

### tert-Butyl (3R)-3-{[5-(2-fluoroethoxy)-1-trityl-1H-indazol-3-yl]carbamoyl}piperidine-1-carboxylate [(XXIV), R2=tert-butyl piperidine-1-carboxylate, R4=fluoroethyl, R9=1,1′,1″-methanetriyltribenzene]

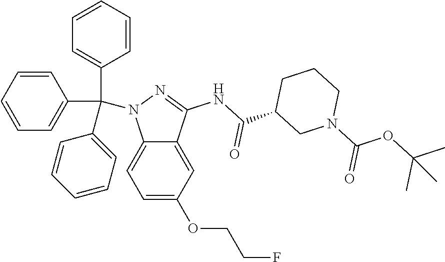 [SMILES: CC(C)(C)OC(=O)N1CCC[C@@H](C(=O)Nc2nn(C(c3ccccc3)(c3ccccc3)c3ccccc3)c3ccc(OCCF)cc23)C1]

To a solution of 1-(tert-butoxycarbonyl)piperidine-3-carboxylic acid (0.188 g, 0.823 mmol) in dry DCM (10 mL), cooled at 0-5° C., 1-chloro-N,N,2-trimethyl-1-propenylamine (Ghosez's reagent) (0.163 mL, 0.1.235 mmol) was added. The mixture was stirred at room temperature for 2 h, then was added dropwise to a solution of 5-(2-fluoroethoxy)-1-trityl-1H-indazol-3-amine (0.30 g, 0.686 mmol) and pyridine (0.20 mL, 2.469 mmol) in dry THF (20 mL), cooled at 0-5° C. The reaction was stirred at room temperature for 3 h, then diluted with DCM (40 mL) and washed with 10% aqueous citric acid (40 mL), water (40 mL) and brine (40 mL). The organic phase was dried with anhydrous Na₂SO₄, filtered and evaporated to dryness. The crude was purified by flash chromatography (Hexane/EtOAc 70:30) to obtain tert-butyl (3R)-3-{[5-(2-fluoroethoxy)-1-trityl-1H-indazol-3-yl]carbamoyl}piperidine-1-carboxylate (0.372 g, 95% of yield) as white solid. ¹H NMR (500 MHz, DMSO-d₆) δ ppm 1.30-1.39 (m, 10H), 1.62-1.72 (m, 2H), 1.96-2.02 (m, 1H) 2.56 (m, 1H), 2.76-2.87 (m, 2H), 3.84 (d, J=14.49 Hz, 1H), 4.08-4.18 (m, 2H), 4.63-4.79 (m, 2H), 6.27 (d, J=9.30 Hz, 1H), 6.77 (dd, J=9.30, 2.44 Hz, 1H), 7.07 (d, J=2.14 Hz, 1H), 7.18 (d, J=7.47 Hz, 6H), 7.33-7.26 (m, 9H), 10.47 (s, 1H). HRMS (ESI) calcd for C39H42FN4O4 [M+H]⁺ 649.3185, found 649.3187.

According to this same methodology, starting from the appropriate intermediates, the following compounds were obtained:

### tert-Butyl (3R)-3-{[5-(2-methoxyethoxy)-1-trityl-1H-indazol-3-yl]carbamoyl}piperidine-1-carboxylate [(XXIV), R2=tert-butyl piperidine-1-carboxylate, R4=methoxyethyl, R9=1,1′,1″-methanetriyltribenzene]

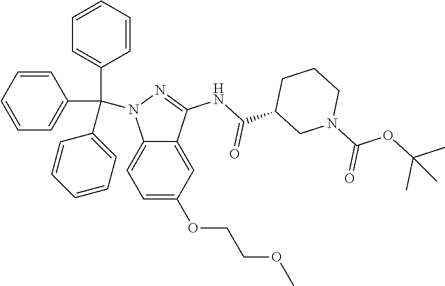 [SMILES: COCCOc1ccc2c(c1)c(NC(=O)[C@@H]1CCCN(C(=O)OC(C)(C)C)C1)nn2C(c1ccccc1)(c1ccccc1)c1ccccc1]

Starting from 5-(2-methoxyethoxy)-1-trityl-1H-indazol-3-amine. Flash column chromatography (Hexane/EtOAc 65:35). Obtained 0.098 g (92% of yield). LCMS (ESI) m/z 661 (M+H)⁺.

### tert-Butyl (3R)-3-[(5-ethoxy-1-trityl-1H-indazol-3-yl)carbamoyl]piperidine-1-carboxylate [(XXIV), R2=tert-butyl piperidine-1-carboxylate, R4=ethyl, R9=1,1′,1″-methanetriyltribenzene]

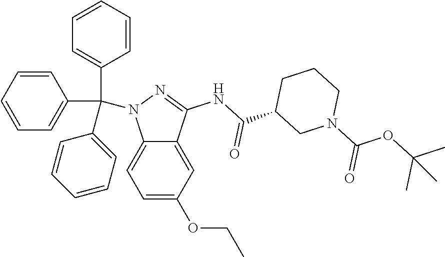 [SMILES: CCOc1ccc2c(c1)c(NC(=O)[C@@H]1CCCN(C(=O)OC(C)(C)C)C1)nn2C(c1ccccc1)(c1ccccc1)c1ccccc1]

Starting from 5-ethoxy-1-trityl-1H-indazol-3-amine. Flash column chromatography (Hexane/EtOAc 70:30). Obtained 0.105 g (97% of yield). LCMS (ESI) m/z 631 (M+H)⁺.

### tert-Butyl (3R)-3-{[5-(propan-2-yloxy)-1-trityl-1H-indazol-3-yl]carbamoyl}piperidine-1-carboxylate [(XXIV), R2=tert-butyl piperidine-1-carboxylate, R4=2-propyl, R9=1,1′,1″-methanetriyltribenzene]

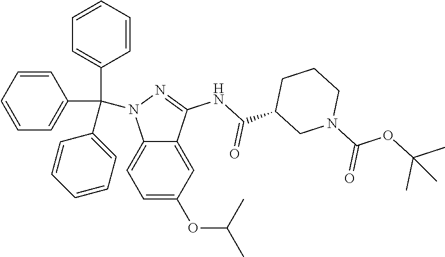 [SMILES: CC(C)Oc1ccc2c(c1)c(NC(=O)[C@@H]1CCCN(C(=O)OC(C)(C)C)C1)nn2C(c1ccccc1)(c1ccccc1)c1ccccc1]

Starting from 5-(propan-2-yloxy)-1-trityl-1H-indazol-3-amine. Flash column chromatography (Hexane/EtOAc 70:30). Obtained 0.173 g (96% of yield). LCMS (ESI) m/z 645 (M+H)⁺.

### tert-Butyl (3R)-3-[(5-butoxy-1-trityl-1H-indazol-3-yl)carbamoyl]piperidine-1-carboxylate [(XXIV), R2=tert-butyl piperidine-1-carboxylate, R4=butyl, R9=1,1′,1″-methanetriyltribenzene]

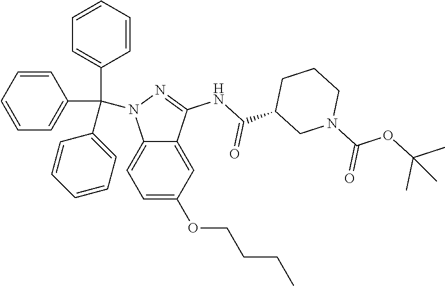 [SMILES: CCCCOc1ccc2c(c1)c(NC(=O)[C@@H]1CCCN(C(=O)OC(C)(C)C)C1)nn2C(c1ccccc1)(c1ccccc1)c1ccccc1]

Starting from 5-butoxy-1-trityl-1H-indazol-3-amine. Flash column chromatography (Hexane/EtOAc 75:25). Obtained 0.123 g (94% of yield). LCMS (ESI) m/z 659 (M+H)⁺.

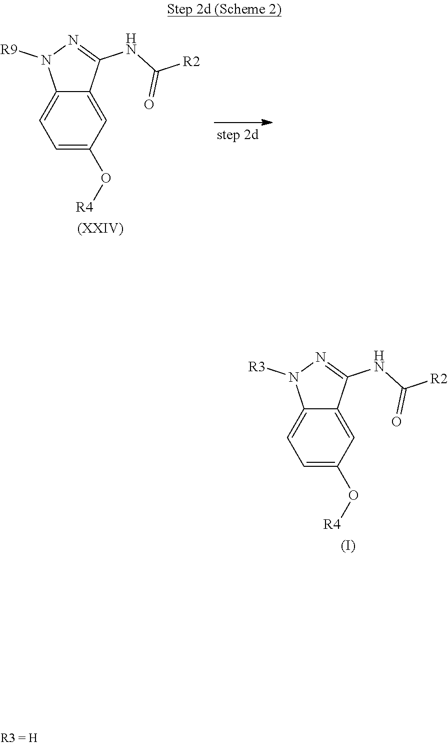

### (3R)-N-[5-(2-Fluoroethoxy)-1H-indazol-3-yl]piperidine-3-carboxamide hydrochloride (cpd 227) [(I), R2=piperidine, R4=fluoroethyl, R3=H]

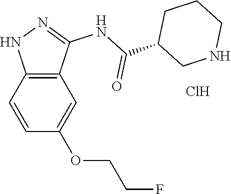 [SMILES: O=C(Nc1n[nH]c2ccc(OCCF)cc12)[C@@H]1CCCNC1 ; Cl]

To a solution of tert-butyl (3R)-3-{[5-(2-fluoroethoxy)-1-trityl-1H-indazol-3-yl]carbamoyl}piperidine-1-carboxylate (0.090 g, 0.139 mmol) in dry DCM (5 mL), triethylsilane (0,041 mL, 0.278 mmol) and 4M HCl in dioxane (0.52 mL, 2.09 mmol) were added. The mixture was stirred at room temperature for 4 h, then the resulting suspension was evaporated to dryness. The crude was treated with MTBE (10 mL), stirred for 10 min. and filtered. After drying at 40° C. under vacuo (3R)-N-[5-(2-fluoroethoxy)-1H-indazol-3-yl]piperidine-3-carboxamide hydrochloride (0.036 g, 75% of yield) was obtained as white solid. ¹H NMR (500 MHz, DMSO-d₆) δ ppm 1.71-1.90 (m, 3H), 2.11 (m, 1H), 2.91-3.01 (m, 2H), 3.10 (q, J=10.07 Hz, 1H), 3.19 (d, J=12.96 Hz, 1H), 3.35 (m, 1H), 4.11-4.27 (m, 2H), 4.67-4.85 (m, 2H), 7.06 (dd, J=9.00, 2.29 Hz, 1H), 7.14 (d, J=2.14 Hz, 1H), 7.38 (d, J=9.00 Hz, 1H), 8.71-8.86 (m, 2H), 10.53 (s, 1H), 12.63 (br. s., 1H). HRMS (ESI) calcd for C15H20FN4O2 [M+H]⁺ 307.1565, found 307.1564. According to this same methodology, starting from the appropriate intermediates, the following compounds were obtained:

### (3R)-N-[5-(2-Methoxyethoxy)-1H-indazol-3-yl]piperidine-3-carboxamide hydrochloride (cpd 228) [(I), R2=piperidine, R4=methoxyethyl, R3=H]

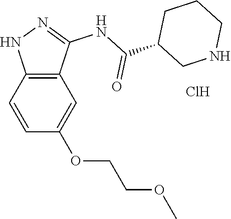 [SMILES: COCCOc1ccc2[nH]nc(NC(=O)[C@@H]3CCCNC3)c2c1 ; Cl]

Obtained 0.011 g (56% of yield). ¹H NMR (500 MHz, DMSO-d₆) δ ppm 1.71-1.77 (m, 2H), 1.85 (m, 1H), 2.11 (m, 1H), 2.91-3.00 (m, 2H), 3.12 (m, 1H), 3.17 (d, J=13.27 Hz, 1H), 3.32 (s, 3H), 3.36 (m, 1H), 3.67 (t, J=4.73 Hz, 2H), 4.04 (t, J=4.73 Hz, 2H), 7.03 (dd, J=9.00, 2.29 Hz, 1H), 7.10 (d, J=2.13 Hz, 1H), 7.36 (d, J=9.00 Hz, 1H), 8.66-8.79 (m, 2H), 10.51 (s, 1H), 12.60 (br. s., 1H). HRMS (ESI) calcd for C16H23N4O3 [M+H]⁺ 319.1765, found 319.1758.

### (3R)-N-(5-Ethoxy-1H-indazol-3-yl)piperidine-3-carboxamide hydrochloride (cpd 229) [(I), R2=piperidine, R4=ethyl, R3=H]

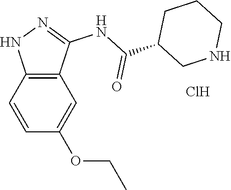 [SMILES: CCOc1ccc2[nH]nc(NC(=O)[C@@H]3CCCNC3)c2c1 ; Cl]

Obtained 0.015 g (81% of yield). 1H NMR (500 MHz, DMSO-d₆) δ ppm 1.34 (t, J=7.02 Hz, 3H), 1.70-1.77 (m, 2H), 1.85 (m, 1H), 2.10 (m, 1H), 2.92-2.99 (m, 2H), 3.12 (m, 1H), 3.19 (d, J=11.90 Hz, 1H), 3.35 (m, 1H), 3.98 (q, J=7.02 Hz, 2H), 7.00 (dd, J=8.92, 2.36 Hz, 1H), 7.08 (d, J=2.14 Hz, 1H), 7.35 (d, J=9.00 Hz, 1H), 8.71 (d, J=16.47 Hz, 2H), 10.50 (s, 1H), 12.58 (br. s., 1H). HRMS (ESI) calcd for C15H21N4O2 [M+H]⁺ 289.1659, found 289.1661.

### (3R)-N-[5-(Propan-2-yloxy)-1H-indazol-3-yl]piperidine-3-carboxamide hydrochloride (cpd 230) [(I), R2=piperidine, R4=2-propyl, R3=H]

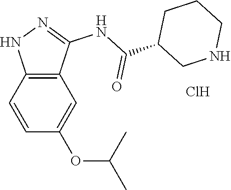 [SMILES: CC(C)Oc1ccc2[nH]nc(NC(=O)[C@@H]3CCCNC3)c2c1 ; Cl]

Obtained 0.033 g (92% of yield). ¹H NMR (500 MHz, DMSO-d₆) δ ppm 1.26 (d, J=5.95 Hz, 6H), 1.68-1.76 (m, 2H), 1.85 (m, 1H), 2.10 (m, 1H), 2.91-2.98 (m, 2H), 3.08-3.14 (m, 1H), 3.19 (d, J=12.20 Hz, 1H), 3.37 (m, 1H), 4.50 (dt, J=12.09, 6.08 Hz, 1H), 6.99 (dd, J=9.00, 2.29 Hz, 1H), 7.11 (d, J=2.14 Hz, 1H), 7.34 (d, J=9.00 Hz, 1H), 8.72 (d, J=19.83 Hz, 2H), 10.50 (s, 1H), 12.57 (br. s., 1H). HRMS (ESI) calcd for C16H23N4O2 [M+H]⁺ 303.1816, found 303.1814.

### (3R)-N-(5-Butoxy-1H-indazol-3-yl)piperidine-3-carboxamide hydrochloride (cpd 231) [(I), R2=piperidine, R4=butyl, R3=H]

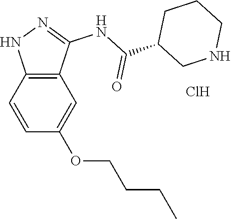 [SMILES: CCCCOc1ccc2[nH]nc(NC(=O)[C@@H]3CCCNC3)c2c1 ; Cl]

Obtained 0.025 g (81% of yield). ¹H NMR (500 MHz, DMSO-d₆) δ ppm 0.94 (t, J=7.40 Hz, 3H), 1.45 (m, 2H), 1.69-1.77 (m, 4H), 1.85 (m, 1H), 2.11 (m, 1H), 2.90-3.00 (m, 2H), 3.11 (m, 1H), 3.18 (d, J=11.29 Hz, 1H), 3.35 (m, 1H), 3.93 (t, J=6.41 Hz, 2H), 7.00 (dd, J=9.00, 2.29 Hz, 1H), 7.09 (d, J=1.98 Hz, 1H), 7.35 (d, J=9.00 Hz, 1H), 8.73 (d, J=19.67 Hz, 2H), 10.49 (s, 1H), 12.58 (br. s., 1H). HRMS (ESI) calcd for C17H25N4O2 [M+H]⁺ 317.1972, found 317.1966.

### Intermediates Preparation

### 3-Bromo-4-cyano-N-methylbenzamide

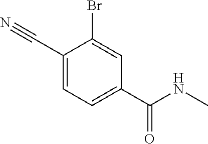

To a solution of 3-bromo-4-cyano-benzoic acid (0.13 g, 0.57 mmol) in dry DCM (7 mL), cooled at 0-5° C., oxalyl chloride (0.097 mL, 0.86 mmol) and catalytic DMF (2 drops) were added. The ice bath was removed and the reaction was stirred at room temperature for 2 h, then the solution was evaporated to dryness. The residue was dissolved in dry THF (8 mL), methylamine 2.0 M in THE (0.86 mL, 1.72 mmol) was added and the resulting mixture was stirred at room temperature overnight. The reaction was diluted with EtOAc (20 mL) and washed with aqueous NaHCO₃ (20 mL), water (20 mL) and brine (20 mL). The organic phase was dried with anhydrous Na₂SO₄, filtered and evaporated to dryness to give 3-bromo-4-cyano-N-methylbenzamide (0.110 g, 81% of yield) that was used for the next step as crude. LCMS (ESI) m/z 238 (M−H)⁻.

### 3-(4-Chloro-3-iodophenyl)-1,2,4-oxadiazole

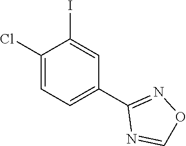 [SMILES: Clc1ccc(-c2ncon2)cc1I]

To a solution of 4-chloro-3-iodo-benzonitrile (0.2 g, 0.76 mmol) in EtOH (5 mL) and water (0.5 mL), hydroxylamine hydrochloride (0.148 g, 2.13 mmol) and K₂CO₃(0.294 g, 2.13 mmol) were added. The mixture was heated at reflux for 5 h, then was cooled, diluted with water (20 mL) and extracted with EtOAc (3×15 mL). The combined organic phase was washed with brine (20 mL), dried with anhydrous Na₂SO₄, filtered and evaporated to dryness to give 4-chloro-N-hydroxy-3-iodo-benzamidine (0.22 g, 96% of yield) that was used for the next step as crude. To this intermediate, triethyl orthoformate (2 mL) was added and the mixture was heated at 140° C. for 8 h. After concentration of the reaction under vacuum the crude was purified by flash column chromatography (hexane/EtOAc 90:10) to give 3-(4-chloro-3-iodophenyl)-1,2,4-oxadiazole (0.119 g, 51% of overall yield) as white solid. ¹H NMR (500 MHz, DMSO-d₆) δ ppm 7.79 (d, J=8.39 Hz, 1H), 8.05 (dd, J=8.31, 2.06 Hz, 1H), 8.51 (d, J=1.98 Hz, 1H), 9.77 (s, 1H). HRMS (ESI) calcd for C8H5ClIN2O [M+H]⁺ 306.9130, found 306.6125.

### 2-(3-Bromo-4-chlorophenyl)-1,3,4-oxadiazole

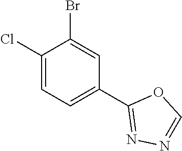 [SMILES: Clc1ccc(-c2nnco2)cc1Br]

A mixture of 3-bromo-4-chlorobenzohydrazide (0.17 g, 0.683 mmol) and triethyl orthoformate (2 mL) was heated at 140° C. for 5 h. After concentration of the reaction under vacuum the crude was purified by flash column chromatography (hexane/EtOAc 80:20) to give 2-(3-bromo-4-chlorophenyl)-1,3,4-oxadiazole (0.13 g, 73% of yield) as brown solid. ¹H NMR (500 MHz, DMSO-d₆) δ ppm 7.88 (d, J=8.39 Hz, 1H), 8.03-8.06 (m, 1H), 8.34 (d, J=1.98 Hz, 1H), 9.42 (s, 1H). HRMS (ESI) calcd for C8H5BrClN2O [M+H]⁺ 258.9269, found 258.9277.

According to this same methodology, starting from the appropriate reagent, the following compound was obtained:

### 2-(3-Bromo-4-chlorophenyl)-5-methyl-1,3,4-oxadiazole

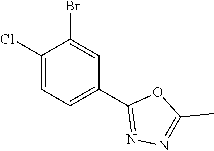

Starting from 3-bromo-4-chlorobenzohydrazide and triethyl orthoacetate. Flash column chromatography (Hexane/EtOAc 70:30). Obtained 0.160 g (73% of yield). ¹H NMR (500 MHz, DMSO-d₆) δ ppm 2.59 (s, 3H), 7.86 (d, J=8.54 Hz, 1H), 7.98 (dd, J=8.31, 2.06 Hz, 1H), 8.27 (d, J=1.98 Hz, 1H). HRMS (ESI) calcd for C9H7BrClN2O [M+H]⁺ 272.9425, found 272.9430.

### Biochemical Kinase Inhibition Assay on CDK11 in Displacement Assay Format

### CDK11A 346-783 Cloning, Protein Expression and Purification

The CDK11A_346-783 DNA fragment was amplified from a Placenta cDNA library and cloned as a cleavable GST fusion protein into a Baculovirus transfer vector for expression in Insect cells. The N-terminal tagged GST-CDK11A_346-783 recombinant protein was expressed in SF21 cells and purified by Affinity-Chromatography on a GSH-column.

The GST tag was removed on column by addition of Prescission protease and resulting cleaved CDK11A_346-783 protein (Uniprot: Q9UQ88-1) was concentrated to reach a final concentration of 2-3 mg/ml. The so concentrated protein was then loaded on a Superdex 200 size exclusion column and the fractions corresponding to the monomeric peak were pooled. At the end, the CDK11A_346-783 protein identity and phosphorylation pattern distribution were analyzed by Mass spectrometry.

### Synthesis of SNS-032-Atto590 Conjugate Probe

SNS-032_Atto590 conjugate probe can be prepared as reported in Scheme 3 below.

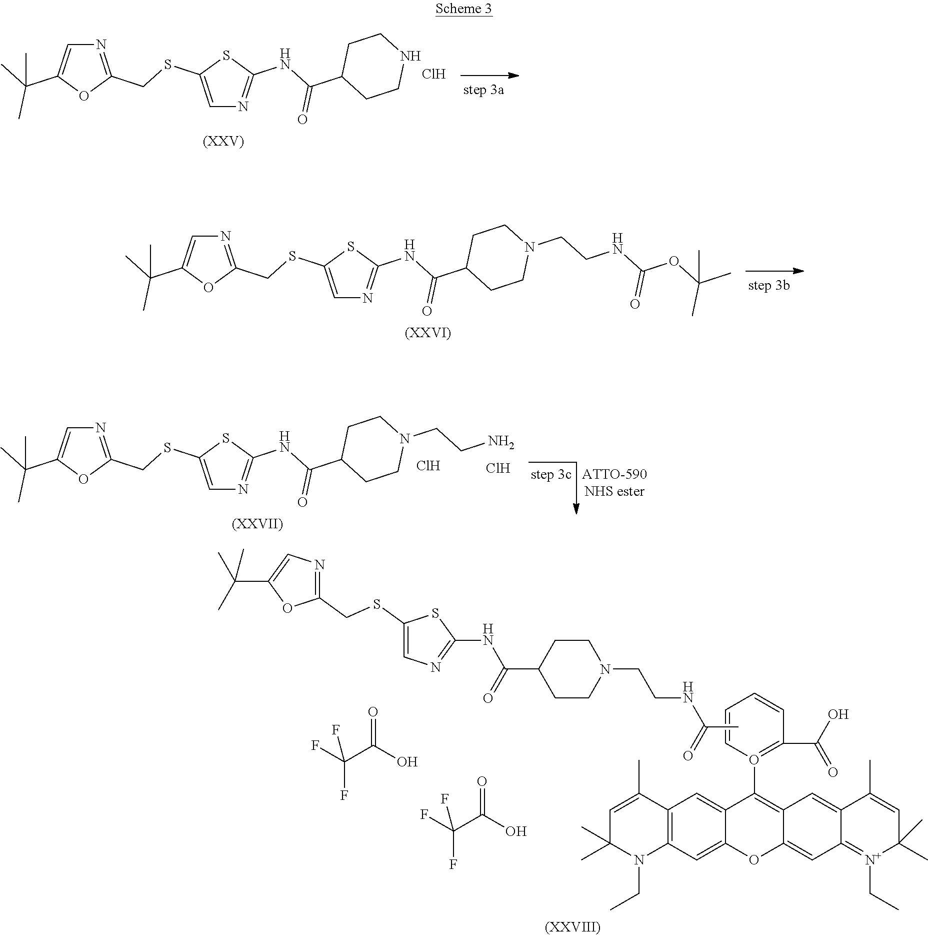

### Step 3a

### tert-Butyl (2-{4-[(5-{[(5-tert-butyl-1,3-oxazol-2-yl)methyl]sulfanyl}-1,3-thiazol-2-yl)carbamoyl]piperidin-1-yl}ethyl)carbamate [XXVI]

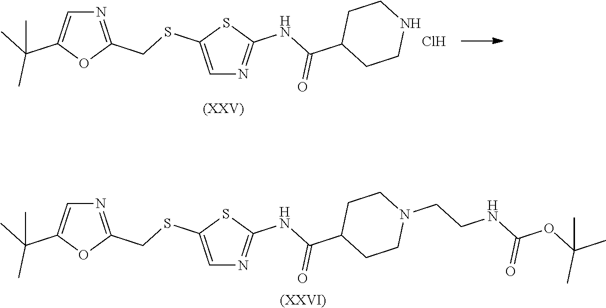

To a solution of piperidine-4-carboxylic acid N-(5-{[(5-tert-butyl-1,3-oxazol-2-yl)methyl]sulfanyl}-1,3-thiazol-2-yl)piperidine-4-carboxamide hydrochloride (SNS-032, MedChemExpress, 0.020 g, 0.048 mmol) in dry DMF (2 mL), tert-butyl (2-oxoethyl)carbamate (0.012 g, 0.072 mmol), tetramethylammonium triacetoxyborohydride (0.038 g, 0.144 mmol) and glacial acetic acid (0.014 mL, 0.240 mmol) were added. The mixture was stirred at room temperature for 1 h, then was diluted with EtOAc (10 mL) and washed with aqueous NaHCO₃ (10 mL), water (10 mL) and brine (10 mL). The organic phase was separated, dried with anhydrous Na₂SO₄, filtered and evaporated. The crude was purified by flash column chromatography (DCM/MeOH 92:8) to give tert-butyl (2-{4-[(5-{[(5-tert-butyl-1,3-oxazol-2-yl)methyl]sulfanyl}-1,3-thiazol-2-yl)carbamoyl]piperidin-1-yl}ethyl)carbamate (0.016 g, 67% of yield) as white solid. LCMS (ESI) m/z 524 (M+H)⁺.

### Step 3b

### 1-(2-Aminoethyl)-N-(5-{[(5-tert-butyl-1,3-oxazol-2-yl)methyl]sulfanyl}-1,3-thiazol-2-yl)piperidine-4-carboxamide dihydrochloride [XXVII]

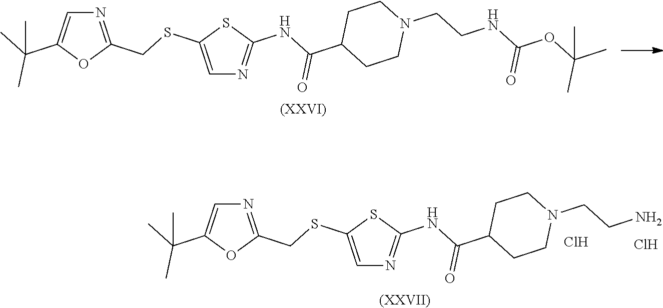

To a solution of tert-butyl (2-{4-[(5-{[(5-tert-butyl-1,3-oxazol-2-yl)methyl]sulfanyl}-1,3-thiazol-2-yl)carbamoyl]piperidin-1-yl}ethyl)carbamate (0.021 g, 0.040 mmol) in dioxane (3 mL), 4M HCl in dioxane (0.3 mL, 1.2 mmol) was added. The reaction was stirred at room temperature overnight. The suspension obtained was evaporated under vacuum to afford 1-(2-aminoethyl)-N-(5-{[(5-tert-butyl-1,3-oxazol-2-yl)methyl]sulfanyl}-1,3-thiazol-2-yl)piperidine-4-carboxamide dihydrochloride (0.020 g, 100% of yield) as ivory solid. ¹H NMR (401 MHz, DMSO-d₆) δ ppm 1.18 (s, 9H), 1.94-2.13 (m, 4H), 2.76 (m, 1H), 2.98-3.05 (m, 2H), 3.24-3.38 (m, 4H), 3.62-3.68 (m, 2H), 4.06 (s, 2H), 6.72 (s, 1H), 7.41 (s, 1H), 8.26 (br. s., 3H), 10.65 (br. s., 1H), 12.42 (s, 1H). HRMS (ESI) calcd for C19H30N5O2S2 [M+H]⁺ 424.1936, found 424.1841.

### Step 3c

### 6-{5-[(2-{4-[(5-{[(5-tert-Butyl-1,3-oxazol-2-yl)methyl]sulfanyl}-1,3-thiazol-2-yl)carbamoyl]piperidin-1-yl)ethyl)-carbamoyl]-2-carboxyphenyl}-1,11-diethyl-2,2,4,8,10,10-hexamethyl-10,11-dihydro-2H-pyrano[3,2-g:5,6-g]diquinolin-1-ium and 6-{6-[(2-{4-[(5-{[(5-tert-butyl-1,3-oxazol-2-yl)methyl]sulfanyl}-1,3-thiazol-2-yl)carbamoyl]-piperidin-1-yl}ethyl)carbamoyl]-2-carboxyphenyl}-1,11-diethyl-2,2,4,8,10,10-hexamethyl-10,11-dihydro-2H-pyrano[3,2-g:5,6-g]diquinolin-1-ium ditrifluoroacetate [XXVIII]

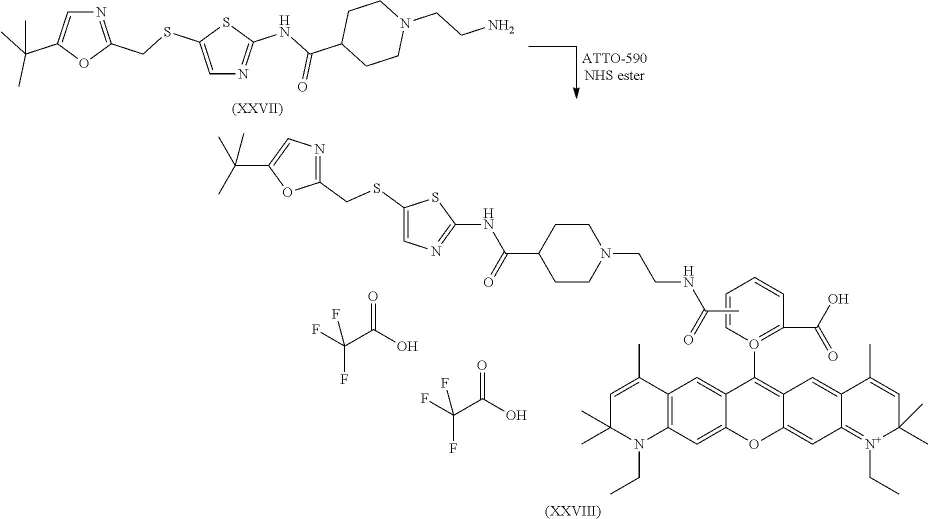

To a solution of Atto 590 NHS ester (mixture of 5- and 6-isomers, 0.001 g, 0.0013 mmol) in dry DMF (0.5 mL), 1-(2-aminoethyl)-N-(5-{[(5-tert-butyl-1,3-oxazol-2-yl)methyl]sulfanyl}-1,3-thiazol-2-yl)piperidine-4-carboxamide dihydrochloride (0.0097 g, 0.002 mmol) and diisopropylethylamine (0.0011 mL, 0.006 mmol) were added. The mixture was stirred at room temperature for 3 h, then the solvent was removed at room temperature under high vacuum. The residue obtained was purified by preparative HPLC to afford 6-{5-[(2-{4-[(5-{[(5-tert-butyl-1,3-oxazol-2-yl)methyl]sulfanyl}-1,3-thiazol-2-yl)carbamoyl]piperidin-1-yl}ethyl)-carbamoyl]-2-carboxyphenyl}-1,11-diethyl-2,2,4,8,10,10-hexamethyl-10,11-dihydro-2H-pyrano[3,2-g:5,6-g]diquinolin-1-ium and 6-{6-[(2-{4-[(5-{[(5-tert-butyl-1,3-oxazol-2-yl)methyl]sulfanyl}-1,3-thiazol-2-yl)carbamoyl]-piperidin-1-yl)ethyl)carbamoyl]-2-carboxyphenyl}-1,11-diethyl-2,2,4,8,10,10-hexamethyl-10,11-dihydro-2H-pyrano[3,2-g:5,6-g]diquinolin-1-ium ditrifluoroacetate (0.0011 g, 69% of yield) as blue solid. LCMS (ESI) m/z 997 (M+H)⁺.

### Displacement Assay Format

Fluorescence Polarization (FP) experiments were carried out at room temperature using no-binding black 384-well microplates (Corning, NY), in 50 mM Tris HCl, pH 8, 10 mM MgCl2, 1 mM DTT, 1 mM CHAPS in a final volume of 60 μl. The plate reader was the Pherastar microplate reader (BMG). Polarization values were read using excitation and emission wavelengths of 590 and 675 nm, respectively. All FP values are expressed as mP units. Displacement experiments were carried out at a constant concentration of SNS-032-Atto590 conjugate probe (0.002 μM) and CDK11A-p58 (0.032 uM), typically equal to its affinity for the probe.

Compounds were 4-folds serially diluted in 100% DMSO and added directly to the CDK11A-p58 solution using the pin tool (V & P Scientific): 0.2 ul of the different compound concentrations in 20 ul of enzyme solution. After an incubation of 30 minutes at r.t., 40 ul of probe solution were added. The final compound dose-curve concentration was from 10 uM up to 0.013 nM. The Pherastar reading is performed after 15 minutes of all components incubation. For each assay, high controls (equivalent to 0% displacement) contain probe, CDK11A-p58, and 0.33% DMSO, whereas low controls (equivalent to 100% displacement) contain only probe and 0.33% DMSO. Controls are used for the Z′ and signal to background evaluation.

All information about plate dilution, distribution, type of assay, targets and raw data of displacement are tracked via barcode reading and stored in an Oracle DB.

The data per each molecule are analyzed by Prism SW or an internally customized version of the SW package “Assay Explorer” which provides sigmoidal fittings of the ten-dilution curves for DC50 determination using a 4 parameters logistic equation:

$$y = bottom + ( top - bottom ) / ( 1 + 1 {0}^{\hat{}} ( ( log DC 50 - x ) * slope ) )$$

where x is the logarithm of the inhibitor concentration, y is the response; y starts at bottom and goes to top with a sigmoid shape.

Table A below, reports the in vitro activity data, performed as reported above, of the compounds of formula (I) against CDK11 kinase. As can be appreciated by the skilled person, most of the compounds show an DC₅₀ value <1.0 μM on CDK11A, and are thus particularly advantageous in therapy against diseases caused by and/or associated with dysregulated CDK11 kinase activity, such as cancer.

TABLE A

| | DC50 |
|---|---|
| Cpd | (μM) |
| 1 | 0.049 |
| 3 | 0.051 |
| 4 | 0.050 |
| 5 | 0.018 |
| 6 | 0.023 |
| 8 | 0.125 |
| 9 | 0.131 |
| 11 | 0.183 |
| 13 | 0.036 |
| 14 | 0.087 |
| 15 | 0.130 |
| 16 | 0.072 |
| 17 | 0.052 |
| 18 | 0.025 |
| 19 | 0.027 |
| 20 | 0.089 |
| 21 | 0.046 |
| 22 | 0.055 |
| 23 | 0.075 |
| 24 | 0.043 |
| 25 | 0.031 |
| 26 | 0.023 |
| 27 | 0.338 |
| 29 | 0.024 |
| 30 | 0.038 |
| 31 | 0.094 |
| 32 | 0.023 |
| 33 | 0.019 |
| 34 | 0.019 |
| 35 | 0.035 |
| 36 | 0.065 |
| 37 | 0.026 |
| 38 | 0.016 |
| 39 | 0.044 |
| 40 | 0.045 |
| 41 | 0.033 |
| 42 | 0.057 |
| 43 | 0.019 |
| 44 | 0.156 |
| 45 | 0.088 |
| 46 | 0.037 |
| 47 | 0.038 |
| 48 | 0.310 |
| 49 | 0.078 |
| 50 | 0.164 |
| 52 | 0.042 |
| 53 | 0.030 |
| 54 | 0.102 |
| 55 | 0.090 |
| 56 | 0.100 |
| 57 | 0.045 |
| 58 | 0.020 |
| 59 | 0.057 |
| 60 | 0.163 |
| 61 | 0.139 |
| 62 | 0.148 |
| 63 | 0.048 |
| 64 | 0.066 |
| 65 | 0.110 |
| 66 | 0.058 |
| 67 | 0.151 |
| 68 | 0.085 |
| 69 | 0.122 |
| 70 | 0.136 |
| 71 | 0.135 |
| 72 | 0.166 |
| 73 | 0.476 |
| 77 | 0.446 |
| 78 | 0.308 |
| 79 | 0.277 |
| 80 | 0.216 |
| 85 | 0.393 |
| 86 | 0.502 |
| 87 | 0.988 |
| 88 | 0.822 |
| 89 | 0.784 |
| 90 | 0.592 |
| 92 | 0.799 |
| 93 | 0.938 |
| 94 | 0.592 |
| 97 | 0.643 |
| 99 | 0.173 |
| 101 | 0.692 |
| 104 | 0.330 |
| 105 | 0.364 |
| 106 | 0.259 |
| 107 | 0.200 |
| 108 | 0.150 |
| 109 | 0.967 |
| 111 | 0.141 |
| 113 | 0.534 |
| 115 | 0.165 |
| 116 | 0.063 |
| 117 | 0.153 |
| 118 | 0.467 |
| 119 | 0.665 |
| 120 | 0.619 |
| 123 | 0.665 |
| 124 | 0.637 |
| 126 | 0.913 |
| 128 | 0.468 |
| 131 | 0.438 |
| 132 | 0.409 |
| 134 | 0.125 |
| 135 | 0.786 |
| 136 | 0.486 |
| 137 | 0.070 |
| 138 | 0.376 |
| 140 | 0.068 |
| 141 | 0.079 |
| 142 | 0.095 |
| 143 | 0.089 |
| 144 | 0.110 |
| 145 | 0.044 |
| 146 | 0.023 |
| 147 | 0.108 |
| 148 | 0.020 |
| 151 | 0.061 |
| 152 | 0.031 |
| 153 | 0.041 |
| 154 | 0.040 |
| 155 | 0.053 |
| 157 | 0.059 |
| 158 | 0.695 |
| 160 | 0.221 |
| 162 | 0.792 |
| 163 | 0.576 |
| 164 | 0.151 |
| 165 | 0.036 |
| 166 | 0.133 |
| 167 | 0.027 |
| 168 | 0.098 |
| 169 | 0.021 |
| 170 | 0.177 |
| 171 | 0.028 |
| 172 | 0.042 |
| 173 | 0.391 |
| 174 | 0.023 |
| 175 | 0.339 |
| 176 | 0.018 |
| 177 | 0.054 |
| 178 | 0.028 |
| 179 | 0.021 |
| 181 | 0.064 |
| 182 | 0.140 |
| 183 | 0.073 |
| 184 | 0.029 |
| 185 | 0.021 |
| 186 | 0.065 |
| 188 | 0.275 |
| 190 | 0.706 |
| 193 | 0.340 |
| 197 | 0.900 |
| 202 | 0.015 |
| 203 | 0.181 |
| 204 | 0.030 |
| 205 | 0.278 |
| 206 | 0.023 |
| 207 | 0.229 |
| 212 | 0.821 |
| 224 | 0.528 |
| 226 | 1.005 |
| 227 | 0.071 |
| 228 | 0.406 |
| 229 | 0.020 |
| 230 | 0.027 |
| 231 | 0.071 |

### Cell Lines and Cell Cultures

The human leukemic cell lines used in this study, their source and growth medium compositions are listed in the Table B below:

TABLE B

| Cell line | Species | Source | Tissue | Tumor type | Culture medium |
|---|---|---|---|---|---|
| Eol-1 | Human | DSMZ | Blood | Leukemia acute myeloid | RPMI 1640 + 2 mM L-Glutamine + 10% FCS |
| MOLM-13 | Human | DSMZ | Blood | Leukemia acute myeloid | RPMI 1640 + 2 mM L-Glutamine + 10-20% FBS |
| NB-4 | HUman | DSMZ | Blood | Leukemia acute myeloid | RPMI 1640 + 2 mM L-Glutamine + 10% FCS |

Exponentially growing cells were seeded and incubated at 37° C. in a humidified 5% CO2 atmosphere using media and maintenance schedules recommended by the supplier.

### Cytotoxicity Assay

All cell lines were seeded at a density ranging from 2,400 to 8,000 cells/well in 384-well cell culture microplates and incubated at 3700 in a humidified 5% 002 atmosphere 24 hours before treatment. Compound solutions were directly administered to the cells using the D300e digital dispenser (Tecan) to obtain scalar final concentrations ranging from 5 nM to 10 μM (duplicate wells for each point), with 80 uL final volume per well. The final DMSO concentration in test wells was adjusted to 0.1% (v/v); control wells were mock-treated and contained the same final concentration of DMSO.

Cell viability was determined after 72 hours treatment by ATP determination in each test well with a luciferase-based detection system (CellTiterGlo, Promega, Madison, WI, USA), following manufacturer's instructions. The intensity of emitted light was measured using an EnVision reader (PerkinElmer, Waltham, MA USA) and expressed as RLU. One to three independent experiments were performed for each cell line, as indicated.

### Analysis of Antiproliferative Data

Cell growth inhibitory activity was evaluated comparing treated versus control data using the Assay Explorer software suite (Symix Technologies Inc.). IC₅₀ₛ values were calculated by sigmoidal fitting interpolation using the following quadriparametric Hill equation:

$$Activity = Bottom + \frac{Top - Bottom}{1 + 10 ( Log {IC}_{50} - Conc ) HillSlope}$$

The Top, Bottom and Hill slope parameters were left free to assume any value, but only curves whose Hill slope had values between 0.5 and 4 were arbitrarily considered acceptable and used to calculate the average IC₅₀ₛ. Curves too steep (HillSlope≥4) or too slight (HillSlope≤0.5) were not included (Table C).

TABLE C

| | Cell line IC₅₀ (μM) | | |
|---|---|---|---|
| Cpd | EoL-1-cell | MOLM-13 | NB-4 |
| 24 | 0.423 | 0.563 | 0.595 |
| 25 | 1.725 | 2.860 | 1.655 |
| 26 | 0.126 | 0.154 | 0.141 |
| 32 | 2.035 | 1.510 | 2.480 |
| 33 | 1.380 | 1.350 | 1.560 |
| 145 | 0.663 | 0.483 | 0.520 |
| 152 | 0.191 | 0.203 | 0.226 |
| 157 | 0.590 | 0.521 | 0.512 |
| 165 | 0.484 | 0.463 | 0.629 |
| 167 | 0.651 | 0.610 | 0.525 |
| 169 | 0.281 | 0.219 | 0.317 |
| 171 | 0.332 | 0.520 | 0.416 |
| 174 | 0.165 | 0.190 | 0.171 |
| 176 | 0.425 | 0.505 | 0.384 |
| 179 | 0.259 | 0.414 | 0.335 |
| 184 | 0.160 | 0.164 | 0.151 |
| 185 | 0.294 | 0.261 | 0.204 |
| 186 | 0.315 | 0.303 | 0.365 |
| 202 | 0.073 | 0.077 | 0.056 |
| 203 | 1.250 | 1.385 | 1.320 |
| 204 | 0.237 | 0.194 | 0.211 |
| 206 | 0.180 | 0.194 | 0.205 |

The data reported above in Table C clearly show that the compounds of the present invention are also active in in vitro proliferation assay against different cell lines, wherein an IC₅₀ activity below 5 micro molar has been found.

1. A compound of formula (I): 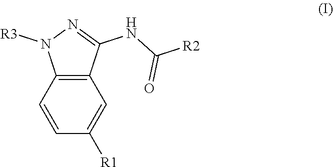
wherein:
R1 is:
optionally substituted straight or branched (C1-C6) alkyl;
optionally substituted (C3-C7) cycloalkyl;
optionally substituted 5 to 7-membered heterocyclyl;
optionally substituted aryl;
5 or 6-membered heteroaryl selected from the group consisting of pyridyl, pyrimidinyl, pyrrolyl and pyrazolyl; or
a group of formula (II): 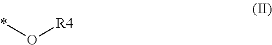
wherein R4 is:
optionally substituted straight or branched (C2-C6) alkyl;
optionally substituted (C3-C7) cycloalkyl; or
optionally substituted 5 to 7-membered heterocyclyl;
R2 is:
straight or branched (C2-C6) alkyl;
optionally substituted (C3-C7) cycloalkyl;
optionally substituted heterocyclyl;
5 to 6-membered heterocyclylmethyl group [—CH2-Het] wherein the heterocyclyl is selected from the group consisting of pirrolydinyl, methylpiperazinyl and piperidinyl; or
a group of formula (III): 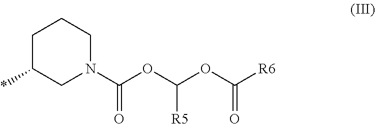
wherein:
R5 is:
hydrogen;
an optionally substituted straight or branched (C1-C6) alkyl;
optionally substituted (C3-C7) cycloalkyl;
optionally substituted aryl;
optionally substituted heterocycly; or
optionally substituted heteroaryl;
R6 is:
an optionally substituted straight or branched (C1-C6) alkyl;
optionally substituted (C3-C7) cycloalkyl;
optionally substituted aryl;
optionally substituted heterocyclyl; or
optionally substituted heteroaryl;
R3 is hydrogen or a group of formula (IV): 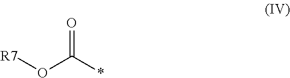
wherein:
R7 is:
an optionally substituted straight or branched (C1-C6) alkyl;
optionally substituted (C3-C7) cycloalkyl;
optionally substituted aryl;
optionally substituted heterocycly; or
optionally substituted heteroaryl;
or pharmaceutically acceptable salt thereof;
provided that R1 is not:

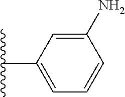 [SMILES: *c1cccc(N)c1]

2. A compound of formula (I) or a pharmaceutically acceptable salt thereof, according to claim 1 wherein:
R1 is:
mono, di or three substituted phenyl;
optionally substituted 5 or 6-membered heteroaryl selected from the group consisting of pyridyl, pyrrolyl and pyrazolyl; wherein the substituents are each independently selected from the group consisting of halogens, straight or branched (C1-C6) alkyl, alkoxy, hydroxyalkyl, polyfluorinated alkyl, polyfluorinated alkoxy, cyano, amino, alkylsulfonyl, aminosulfonyl, phenyl, hydroxy, aminocarbonyl, alkylaminocarbonyl, hydroxyalkylaminocarbonyl, formyl, formylamino, nitro, alkylcarbonyl, alkyloxycarbonyl and oxadiazolyl; or
a group of formula (II): 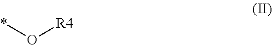
wherein R4 is:
straight or branched (C2-C6) alkyl, C3-C7) cycloalkyl, optionally substituted by halogen or alkoxy;
R2 is:
(C3-C7) cycloalkyl optionally substituted with hydroxy, aminoalkyl, alkylamino, dialkylamino, alkylaminoalkyl, heterocyclyl, C3-C7cycloalkylamino, haloalkylamino;
optionally substituted 5 to 7-membered heterocyclyl with from 1 to 2 heteroatoms selected among N or 0;
1-azabicyclo[2.2.2]octanyl;
2-azabicyclo[2.2.2]octanyl; or
a group of formula (III): 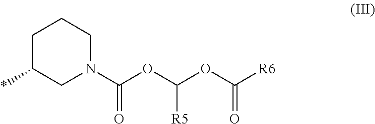
wherein R5 is:
hydrogen;
optionally substituted straight or branched (C1-C6) alkyl; or
optionally substituted (C3-C7) cycloalkyl;
R6 is:
optionally substituted straight or branched (C1-C6) alkyl;
optionally substituted (C3-C7) cycloalkyl;
optionally substituted phenyl;
optionally substituted heterocyclyl; or
optionally substituted 5 or 6-membered heteroaryl with from 1 to 2 nitrogen atoms;
R3 is:
hydrogen or a group of formula (IV) wherein R7 is:
straight or branched (C1-C6) alkyl, optionally substituted with (C3-C7) cycloalkyl,
alkylthio, alkoxy or
alkoxy (C1-C6) alkoxy; or
(C3-C7) cycloalkyl;
provided that R1 is not

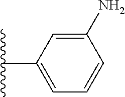 [SMILES: *c1cccc(N)c1]

3. A compound of formula (I) or a pharmaceutically acceptable salt thereof, according to claim 2 wherein:
R1 is:
a disubstituted phenyl;
optionally substituted 5 or 6-membered heteroaryl selected from the group consisting of pyridyl and pyrazolyl;
wherein the substituents are each independently selected from the group consisting of halogens, straight or branched (C1-C6) alkyl alkoxy, hydroxyalkyl, polyfluorinated alkyl, polyfluorinated alkoxy, cyano, amino, alkylsulfonyl, aminosulfonyl, phenyl, hydroxy, aminocarbonyl, alkylaminocarbonyl, hydroxyalkylaminocarbonyl, formyl, formylamino, nitro, alkylcarbonyl, alkyloxycarbonyl and oxadiazolyl; or
a group of formula (II);
R2 is:
(C3-C7) cycloalkyl selected from the group consisting of cyclopropyl, cyclobutyl and cyclohexyl, optionally substituted with hydroxy, aminoalkyl, alkylamino, dialkylamino, haloalkylamino, alkylaminoalkyl, morpholinyl, (C3-C7)cycloalkylamino;
heterocyclyl selected from the group consisting of pyrrolidinyl, piperidinyl, morfolinyl, tetrahydropyranyl and azepanyl, optionally substituted with straight or branched (C1-C6) alkyl, (C3-C6) cycloalkyl, spiro (C4-C6) cycloalkyl, aminoalkyl, alkoxyalkyl, hydroxyalkyl, phenyl, polyfluorinated alkyl, C3-C7 cycloalkylamino and hydroxy; or
a group of formula (III).

4. A compound or a pharmaceutically acceptable salt thereof according to claims 1, 2 or 3, selected from the group consisting of:
N-[5-(4-Amino-2-chlorophenyl)-1H-indazol-3-yl]piperidine-3-carboxamide dihydrochloride (cpd 1);
N-[5-(2,4-Dichlorophenyl)-1H-indazol-3-yl]-1-methylpiperidine-3-carboxamide hydrochloride (cpd 2);
N-[5-(2-Chloro-4-hydroxyphenyl)-1H-indazol-3-yl]piperidine-3-carboxamide hydrochloride (cpd 3);
N-[5-(2,5-Dichlorophenyl)-1H-indazol-3-yl]piperidine-3-carboxamide hydrochloride (cpd 4);
(3R)-N-[5-(4-Amino-2-chlorophenyl)-1H-indazol-3-yl]piperidine-3-carboxamide dihydrochloride (cpd 5);
(3R)-N-[5-(2-chloro-4-hydroxyphenyl)-1H-indazol-3-yl]piperidine-3-carboxamide hydrochloride (cpd 6);
(3S)-N-[5-(4-Amino-2-chlorophenyl)-1H-indazol-3-yl]piperidine-3-carboxamide dihydrochloride (cpd 7);
(3R)-N-[5-(2-Chloro-4-methylphenyl)-1H-indazol-3-yl]piperidine-3-carboxamide hydrochloride (cpd 8);
N-[5-(5-Carbamoyl-2-chlorophenyl)-1H-indazol-3-yl]piperidine-3-carboxamide hydrochloride (cpd 9);
(3S)-N-[5-(2-chloro-4-hydroxyphenyl)-1H-indazol-3-yl]piperidine-3-carboxamide hydrochloride (cpd 10);
N-[5-(3-Amino-2-fluorophenyl)-1H-indazol-3-yl]piperidine-3-carboxamide hydrochloride (cpd 11);
(3S)-N-[5-(2-Chloro-4-methylphenyl)-1H-indazol-3-yl]piperidine-3-carboxamide hydrochloride (cpd 12);
(3R)-N-[5-(2,5-Dichlorophenyl)-1H-indazol-3-yl]piperidine-3-carboxamide hydrochloride (cpd 13);
(3R)-N-[5-(2,4-Dichlorophenyl)-1H-indazol-3-yl]piperidine-3-carboxamide hydrochloride (cpd 14);
N-{5-[5-Chloro-2-(trifluoromethyl)phenyl]-1H-indazol-3-yl}piperidine-3-carboxamide trifluoroacetate (cpd 15);
N-[5-(5-Acetyl-2-fluorophenyl)-1H-indazol-3-yl]piperidine-3-carboxamide hydrochloride (cpd 16);
N-[5-(2-Chloro-5-methoxyphenyl)-1H-indazol-3-yl]piperidine-3-carboxamide hydrochloride (cpd 17);
(3R)-N-[5-(2-Chloro-5-methoxyphenyl)-1H-indazol-3-yl]piperidine-3-carboxamide hydrochloride (cpd 18);
(3R)-N-[5-(5-Acetyl-2-fluorophenyl)-1H-indazol-3-yl]piperidine-3-carboxamide hydrochloride (cpd 19);
(3R)-N-[5-(2-Fluoro-5-methoxyphenyl)-1H-indazol-3-yl]piperidine-3-carboxamide hydrochloride (cpd 20);
(3R)-N-[5-(5-Chloro-2-fluorophenyl)-1H-indazol-3-yl]piperidine-3-carboxamide hydrochloride (cpd 21);
(3R)-N-[5-(5-Ethoxy-2-fluorophenyl)-1H-indazol-3-yl]piperidine-3-carboxamide hydrochloride (cpd 22);
(3R)-N-{5-[2-Fluoro-5-(trifluoromethoxy)phenyl]-1H-indazol-3-yl}piperidine-3-carboxamide hydrochloride (cpd 23);
(3R)-N-[5-(5-Cyano-2-methylphenyl)-1H-indazol-3-yl]piperidine-3-carboxamide hydrochloride (cpd 24);
(3R)-N-[5-(5-Cyano-2-fluorophenyl)-1H-indazol-3-yl]piperidine-3-carboxamide hydrochloride (cpd 25);
(3R)-N-[5-(2-Chloro-5-nitrophenyl)-1H-indazol-3-yl]piperidine-3-carboxamide hydrochloride (cpd 26);
N-[5-(2,6-Dichlorophenyl)-1H-indazol-3-yl]-6-oxopiperidine-3-carboxamide (cpd 27);
N-[5-(5-Acetyl-2-fluorophenyl)-1H-indazol-3-yl]-6-oxopiperidine-3-carboxamide (cpd 28);
Methyl 4-chloro-3-(3-{[(3R)-piperidin-3-ylcarbonyl]amino}-1H-indazol-5-yl)benzoate hydrochloride (cpd 29);
(3R)-N-[5-(2,4-Difluorophenyl)-1H-indazol-3-yl]piperidine-3-carboxamide hydrochloride (cpd 30);
(3R)-N-{5-[4-Methoxy-2-(trifluoromethyl)phenyl]-1H-indazol-3-yl}piperidine-3-carboxamide hydrochloride (cpd 31);
(3R)-N-[5-(2-Fluoro-5-methylphenyl)-1H-indazol-3-yl]piperidine-3-carboxamide hydrochloride (cpd 32);
(3R)-N-[5-(5-Acetyl-2-chlorophenyl)-1H-indazol-3-yl]piperidine-3-carboxamide hydrochloride (cpd 33);
(3R)-N-[5-(5-Amino-2-chlorophenyl)-1H-indazol-3-yl]piperidine-3-carboxamide dihydrochloride (cpd 34);
(3R)-N-[5-(2,5-Difluorophenyl)-1H-indazol-3-yl]piperidine-3-carboxamide hydrochloride (cpd 35);
(3R)-N-{5-[4-Methyl-2-(trifluoromethyl)phenyl]-1H-indazol-3-yl}piperidine-3-carboxamide hydrochloride (cpd 36);
Methyl 4-chloro-3-(3-{1[(3R)-pyrrolidin-3-ylcarbonyl]amino}-1H-indazol-5-yl)benzoate hydrochloride (cpd 37);
(3R)-N-[5-(5-Acetyl-2-chlorophenyl)-1H-indazol-3-yl]pyrrolidine-3-carboxamide hydrochloride (cpd 38);
(3R)-N-{5-[2-Chloro-5-(methylcarbamoyl)phenyl]-1H-indazol-3-yl}pyrrolidine-3-carboxamide hydrochloride (cpd 39);
Methyl 4-cyano-3-(3-{[(3R)-piperidin-3-ylcarbonyl]amino}-1H-indazol-5-yl)benzoate hydrochloride (cpd 40);
(3R)-N-[5-(2-Chloro-5-hydroxyphenyl)-1H-indazol-3-yl]piperidine-3-carboxamide hydrochloride (cpd 41);
Methyl 2,4-dichloro-5-(3-{[(3R)-piperidin-3-ylcarbonyl]amino}-1H-indazol-5-yl)benzoate hydrochloride (cpd 42);
(3R)-N-[5-(2-Chloro-5-propanoylphenyl)-1H-indazol-3-yl]piperidine-3-carboxamide hydrochloride (cpd 43);
(3R)-N-{5-[2-Chloro-4-(trifluoromethyl)phenyl]-1H-indazol-3-yl}piperidine-3-carboxamide hydrochloride (cpd 44);
(3R)-N-{5-[4-Chloro-2-(trifluoromethyl)phenyl]-1H-indazol-3-yl}piperidine-3-carboxamide hydrochloride (cpd 45);
(3R)-N-{5-[5-Fluoro-2-(trifluoromethyl)phenyl]-1H-indazol-3-yl}piperidine-3-carboxamide hydrochloride (cpd 46);
(3R)-N-[5-(2-Chlorophenyl)-1H-indazol-3-yl]piperidine-3-carboxamide hydrochloride (cpd 47);
(3R)-N-{5-[2-Chloro-5-(propan-2-yl)phenyl]-1H-indazol-3-yl}piperidine-3-carboxamide hydrochloride (cpd 48);
(3R)-N-(5-Phenyl-1H-indazol-3-yl)piperidine-3-carboxamide hydrochloride (cpd 49);
Methyl 2,4-difluoro-5-(3-{[(3R)-piperidin-3-ylcarbonyl]amino}-1H-indazol-5-yl)benzoate hydrochloride (cpd 50);
(3R)-N-[5-(5-tert-Butyl-2-chlorophenyl)-1H-indazol-3-yl]piperidine-3-carboxamide hydrochloride (cpd 51);
(3R)-N-[5-(2-Chloro-5-fluorophenyl)-1H-indazol-3-yl]piperidine-3-carboxamide hydrochloride (cpd 52);
(3R)-N-[5-(2,5-Dicyanophenyl)-1H-indazol-3-yl]piperidine-3-carboxamide hydrochloride (cpd 53);
Methyl 2-amino-4-chloro-5-(3-{[(3R)-pyrrolidin-3-ylcarbonyl]amino}-1H-indazol-5-yl)benzoate dihydrochloride (cpd 54);
(3R)-N-{5-[2-Cyano-5-(methylcarbamoyl)phenyl]-1H-indazol-3-yl}piperidine-3-carboxamide hydrochloride (cpd 55);
Propan-2-yl 4-chloro-3-(3-{[(3R)-piperidin-3-ylcarbonyl]amino}-1H-indazol-5-yl)benzoate hydrochloride (cpd 56);
Methyl 2,4-dichloro-3-(3-{[(3R)-piperidin-3-ylcarbonyl]amino}-1H-indazol-5-yl)benzoate hydrochloride (cpd 57);
(3R)-N-{5-[2-Chloro-5-(1,2,4-oxadiazol-3-yl)phenyl]-1H-indazol-3-yl}piperidine-3-carboxamide dihydrochloride (cpd 58);
(3R)-N-{5-[2-Chloro-5-(1,3,4-oxadiazol-2-yl)phenyl]-1H-indazol-3-yl}piperidine-3-carboxamide trifluoroacetate (cpd 59);
(3R)-N-{5-[2-Chloro-5-(5-methyl-1,2,4-oxadiazol-3-yl)phenyl]-1H-indazol-3-yl}piperidine-3-carboxamide dihydrochloride (cpd 60);
N-[5-(2-Chloro-5-cyanophenyl)-1H-indazol-3-yl]piperidine-4-carboxamide hydrochloride (cpd 61);
N-[5-(2-Chloro-4-hydroxyphenyl)-1H-indazol-3-yl]-3-(dimethylamino)cyclobutanecarboxamide hydrochloride (cpd 62);
N-[5-(2-Chloro-4-hydroxyphenyl)-1H-indazol-3-yl]-3-(methylamino)cyclobutanecarboxamide hydrochloride (cpd 63);
N-[5-(2-Chloro-6-fluorophenyl)-1H-indazol-3-yl]-1-methylpiperidine-4-carboxamide hydrochloride (cpd 64);
1-Butyl-N-[5-(2-chloro-6-fluorophenyl)-1H-indazol-3-yl]piperidine-4-carboxamide hydrochloride (cpd 65);
1-(3-Aminopropyl)-N-[5-(2-chloro-6-fluorophenyl)-1H-indazol-3-yl]piperidine-4-carboxamide dihydrochloride (cpd 66);
N-[5-(2-Chloro-6-fluorophenyl)-1H-indazol-3-yl]-1-(1-methoxypropan-2-yl)piperidine-4-carboxamide hydrochloride (cpd 67);
N-[5-(2,6-Dichlorophenyl)-1H-indazol-3-yl]-1-methylpiperidine-4-carboxamide (cpd 68);
1-Butyl-N-[5-(2,6-dichlorophenyl)-1H-indazol-3-yl]piperidine-4-carboxamide (cpd 69);
N-[5-(2,6-Dichlorophenyl)-1H-indazol-3-yl]-1-(2-methylpropyl)piperidine-4-carboxamide (cpd 70);
N-[5-(2,6-Dichlorophenyl)-1H-indazol-3-yl]-1-(3-hydroxy-2,2-dimethylpropyl)piperidine-4-carboxamide (cpd 71);
N-[5-(6-Chloro-2-fluoro-3-methylphenyl)-1H-indazol-3-yl]-1-methylpiperidine-4-carboxamide hydrochloride (cpd 72);
N-[5-(2-Chloro-6-fluoro-3-methylphenyl)-1H-indazol-3-yl]-1-methylpiperidine-4-carboxamide hydrochloride (cpd 73);
N-[5-(5-Chloro-2-fluoro-3-methylphenyl)-1H-indazol-3-yl]-1-methylpiperidine-4-carboxamide hydrochloride (cpd 74);
N-{5-[2-Methoxy-5-(propan-2-yl)phenyl]-1H-indazol-3-yl}-1-methylpiperidine-4-carboxamide hydrochloride (cpd 75);
1-Methyl-N-{5-[3-(trifluoromethyl)phenyl]-1H-indazol-3-yl}piperidine-4-carboxamide hydrochloride (cpd 76);
N-{5-[2-Chloro-5-(hydroxymethyl)phenyl]-1H-indazol-3-yl}-1-methylpiperidine-4-carboxamide p-toluensulfonate (cpd 77);
N-[5-(2-Fluorophenyl)-1H-indazol-3-yl]-1-methylpiperidine-4-carboxamide hydrochloride (cpd 78);
N-[5-(2-Cyanophenyl)-1H-indazol-3-yl]-1-methylpiperidine-4-carboxamide p-toluensulfonate (cpd 79);
N-[5-(2,5-Dichlorophenyl)-1H-indazol-3-yl]-1-methylpiperidine-4-carboxamide hydrochloride (cpd 80);
N-[5-(3-Carbamoylphenyl)-1H-indazol-3-yl]-1-methylpiperidine-4-carboxamide hydrochloride (cpd 81);
N-{5-[4-(Hydroxymethyl)phenyl]-1H-indazol-3-yl}-1-methylpiperidine-4-carboxamide p-toluensulfonate (cpd 82);
N-{5-[3-(Hydroxymethyl)phenyl]-1H-indazol-3-yl}-1-methylpiperidine-4-carboxamide p-toluensulfonate (cpd 83);
1-Methyl-N-[5-(pyridin-3-yl)-1H-indazol-3-yl]piperidine-4-carboxamide dihydrochloride (cpd 84);
N-[5-(2-Chlorophenyl)-1H-indazol-3-yl]-1-methylpiperidine-4-carboxamide hydrochloride (cpd 85);
N-[5-(2-Chloro-5-methylphenyl)-1H-indazol-3-yl]-1-methylpiperidine-4-carboxamide hydrochloride (cpd 86);
N-[5-(4-Fluorophenyl)-1H-indazol-3-yl]-1-methylpiperidine-4-carboxamide hydrochloride (cpd 87);
N-[5-(2-Ethylphenyl)-1H-indazol-3-yl]-1-methylpiperidine-4-carboxamide hydrochloride (cpd 88);
N-[5-(3-Cyanophenyl)-1H-indazol-3-yl]-1-methylpiperidine-4-carboxamide hydrochloride (cpd 89);
N-[5-(4-Aminophenyl)-1H-indazol-3-yl]-1-methylpiperidine-4-carboxamide dihydrochloride (cpd 90);
N-[5-(2-Aminophenyl)-1H-indazol-3-yl]-1-methylpiperidine-4-carboxamide dihydrochloride (cpd 91);
1-Methyl-N-[5-(4-sulfamoylphenyl)-1H-indazol-3-yl]piperidine-4-carboxamide hydrochloride (cpd 92);
1-Methyl-N-(5-phenyl-1H-indazol-3-yl)piperidine-4-carboxamide hydrochloride (cpd 93);
N-{5-[2-Chloro-5-(trifluoromethyl)phenyl]-1H-indazol-3-yl}-1-methylpiperidine-4-carboxamide hydrochloride (cpd 94);
N-[5-(2-Fluoropyridin-3-yl)-1H-indazol-3-yl]-1-methylpiperidine-4-carboxamide dihydrochloride (cpd 95);
N-[5-(2-Methoxyphenyl)-1H-indazol-3-yl]-1-methylpiperidine-4-carboxamide hydrochloride (cpd 96);
N-[5-(2,3-Dichlorophenyl)-1H-indazol-3-yl]-1-methylpiperidine-4-carboxamide hydrochloride (cpd 97);
N-[5-(2-Chloro-6-methoxyphenyl)-1H-indazol-3-yl]-1-methylpiperidine-4-carboxamide hydrochloride (cpd 98);
N-[5-(2,3-Difluorophenyl)-1H-indazol-3-yl]-1-methylpiperidine-4-carboxamide hydrochloride (cpd 99);
1-Methyl-N-{5-[3-(methylsulfonyl)phenyl]-1H-indazol-3-yl}piperidine-4-carboxamide hydrochloride (cpd 100);
N-[5-(3-Fluorophenyl)-1H-indazol-3-yl]-1-methylpiperidine-4-carboxamide hydrochloride (cpd 101);
N-[5-(2-Hydroxyphenyl)-1H-indazol-3-yl]-1-methylpiperidine-4-carboxamide hydrochloride (cpd 102);
N-[5-(Biphenyl-2-yl)-1H-indazol-3-yl]-1-methylpiperidine-4-carboxamide hydrochloride (cpd 103);
1-Methyl-N-{5-[2-(trifluoromethyl)phenyl]-1H-indazol-3-yl}piperidine-4-carboxamide hydrochloride (cpd 104);
N-[5-(2,6-Difluorophenyl)-1H-indazol-3-yl]-1-methylpiperidine-4-carboxamide hydrochloride (cpd 105);
N-[5-(2,4-Dichlorophenyl)-1H-indazol-3-yl]-1-methylpiperidine-4-carboxamide hydrochloride (cpd 106);
N-[5-(4-Amino-2-chlorophenyl)-1H-indazol-3-yl]-1-methylpiperidine-4-carboxamide dihydrochloride (cpd 107);
1-Methyl-N-[5-(2,4,6-trichlorophenyl)-1H-indazol-3-yl]piperidine-4-carboxamide hydrochloride (cpd 108);
N-[5-(4-Carbamoylphenyl)-1H-indazol-3-yl]-1-methylpiperidine-4-carboxamide hydrochloride (cpd 109);
N-[5-(4-Cyanophenyl)-1H-indazol-3-yl]-1-methylpiperidine-4-carboxamide hydrochloride (cpd 110);
N-[5-(2-Chloro-4-hydroxyphenyl)-1H-indazol-3-yl]-1-methylpiperidine-4-carboxamide hydrochloride (cpd 111);
N-[5-(4-Amino-3-fluorophenyl)-1H-indazol-3-yl]-1-methylpiperidine-4-carboxamide hydrochloride (cpd 112);
N-[5-(4-Cyano-2-methylphenyl)-1H-indazol-3-yl]-1-methylpiperidine-4-carboxamide hydrochloride (cpd 113);
N-[5-(2,6-Dimethylphenyl)-1H-indazol-3-yl]-1-methylpiperidine-4-carboxamide trifluoroacetate (cpd 114);
N-[5-(2-Fluoro-5-nitrophenyl)-1H-indazol-3-yl]-1-methylpiperidine-4-carboxamide hydrochloride (cpd 115);
N-[5-(2-Chloro-5-cyanophenyl)-1H-indazol-3-yl]-1-methylpiperidine-4-carboxamide trifluoroacetate (cpd 116);
N-[5-(5-Carbamoyl-2-chlorophenyl)-1H-indazol-3-yl]-1-methylpiperidine-4-carboxamide trifluoroacetate (cpd 117);
N-[5-(2-Fluoro-4-formylphenyl)-1H-indazol-3-yl]-1-methylpiperidine-4-carboxamide hydrochloride (cpd 118);
N-[5-(2-Fluoro-4-methylphenyl)-1H-indazol-3-yl]-1-methylpiperidine-4-carboxamide hydrochloride (cpd 119);
N-[5-(2,6-Difluoro-4-methoxyphenyl)-1H-indazol-3-yl]-1-methylpiperidine-4-carboxamide hydrochloride (cpd 120);
1-Methyl-N-[5-(1H-pyrazol-4-yl)-1H-indazol-3-yl]piperidine-4-carboxamide hydrochloride (cpd 121);
1-Methyl-N-[5-(1-methyl-1H-pyrazol-4-yl)-1H-indazol-3-yl]piperidine-4-carboxamide hydrochloride (cpd 122);
1-Methyl-N-[5-(2,4,6-trifluorophenyl)-1H-indazol-3-yl]piperidine-4-carboxamide hydrochloride (cpd 123);
N-[5-(2,4-Difluorophenyl)-1H-indazol-3-yl]-1-methylpiperidine-4-carboxamide hydrochloride (cpd 124);
1-Methyl-N-[5-(1H-pyrazol-3-yl)-1H-indazol-3-yl]piperidine-4-carboxamide hydrochloride (cpd 125);
1-Methyl-N-[5-(2,4,5-trifluorophenyl)-1H-indazol-3-yl]piperidine-4-carboxamide hydrochloride (cpd 126);
1-Methyl-N-{5-[2,4,6-trifluoro-3-(propan-2-yloxy)phenyl]-1H-indazol-3-yl}piperidine-4-carboxamide hydrochloride (cpd 127);
1-Methyl-N-[5-(2,3,4-trifluorophenyl)-1H-indazol-3-yl]piperidine-4-carboxamide hydrochloride (cpd 128);
N-[5-(4-fluoro-2-methylphenyl)-1H-indazol-3-yl]-1-methylpiperidine-4-carboxamide trifluoroacetate (cpd 129);
N-[5-(4-Amino-2-methylphenyl)-1H-indazol-3-yl]-1-methylpiperidine-4-carboxamide dihydrochloride (cpd 130);
1-Methyl-N-[5-(2,4,6-trifluoro-3-methoxyphenyl)-1H-indazol-3-yl]piperidine-4-carboxamide hydrochloride (cpd 131);
N-[5-(2,3-Difluoro-4-methylphenyl)-1H-indazol-3-yl]-1-methylpiperidine-4-carboxamide hydrochloride (cpd 132);
N-[5-(2-Chloro-4-fluoro-3-methylphenyl)-1H-indazol-3-yl]-1-methylpiperidine-4-carboxamide hydrochloride (cpd 133);
N-[5-(3-Cyano-2,6-difluorophenyl)-1H-indazol-3-yl]-1-methylpiperidine-4-carboxamide (cpd 134);
N-[5-(3-Cyano-4-hydroxyphenyl)-1H-indazol-3-yl]-1-methylpiperidine-4-carboxamide hydrochloride (cpd 135);
N-[5-(2-Cyano-5-fluorophenyl)-1H-indazol-3-yl]-1-methylpiperidine-4-carboxamide hydrochloride (cpd 136);
N-[5-(2-Chloro-5-nitrophenyl)-1H-indazol-3-yl]-1-methylpiperidine-4-carboxamide trifluoroacetate (cpd 137);
N-[5-(2,6-Difluoro-4-hydroxyphenyl)-1H-indazol-3-yl]-1-methylpiperidine-4-carboxamide trifluoroacetate (cpd 138);
1-Methyl-N-[5-(2,4,6-trifluoro-3-hydroxyphenyl)-1H-indazol-3-yl]piperidine-4-carboxamide (cpd 139);
1-Butyl-N-[5-(2-chloro-5-cyanophenyl)-1H-indazol-3-yl]piperidine-4-carboxamide hydrochloride (cpd 140);
N-[5-(2-Chloro-5-cyanophenyl)-1H-indazol-3-yl]-1-propylpiperidine-4-carboxamide hydrochloride (cpd 141);
N-[5-(2-Chloro-5-cyanophenyl)-1H-indazol-3-yl]-1-pentylpiperidine-4-carboxamide hydrochloride (cpd 142);
N-[5-(2-Chloro-5-cyanophenyl)-1H-indazol-3-yl]-1-(propan-2-yl)piperidine-4-carboxamide hydrochloride (cpd 143);
N-[5-(2,6-Dichlorophenyl)-1H-indazol-3-yl]-4-(methylamino)cyclohexanecarboxamide hydrochloride (cpd 144);
N-[5-(2,6-Dichlorophenyl)-1H-indazol-3-yl]-3-(methylamino)cyclobutanecarboxamide hydrochloride (cpd 145);
N-[5-(2-Chloro-5-cyanophenyl)-1H-indazol-3-yl]-3-(methylamino)cyclobutanecarboxamide hydrochloride (cpd 146);
N-[5-(2-Chloro-5-cyanophenyl)-1H-indazol-3-yl]-3-(dimethylamino)cyclobutanecarboxamide hydrochloride (cpd 147);
N-[5-(2-Chloro-5-cyanophenyl)-1H-indazol-3-yl]-3-(propan-2-ylamino)cyclobutanecarboxamide hydrochloride (cpd 148);
N-[5-(2-Chloro-5-cyanophenyl)-1H-indazol-3-yl]-3-(morpholin-4-yl)cyclobutanecarboxamide hydrochloride (cpd 149);
N-[5-(2-Chloro-5-cyanophenyl)-1H-indazol-3-yl]-3-[(2,2,2-trifluoroethyl)amino]cyclobutanecarboxamide hydro-chloride (cpd 150);
N-[5-(2-Chloro-5-cyanophenyl)-1H-indazol-3-yl]-3-[(2-fluoroethyl)amino]cyclobutanecarboxamide hydrochloride (cpd 151);
N-[5-(2-Chloro-5-cyanophenyl)-1H-indazol-3-yl]-3-(ethylamino)cyclobutanecarboxamide hydrochloride (cpd 152);
N-[5-(2-Chloro-5-cyanophenyl)-1H-indazol-3-yl]-3-(cyclobutylamino)cyclobutanecarboxamide hydrochloride (cpd 153);
N-[5-(2-Chloro-5-cyanophenyl)-1H-indazol-3-yl]-3-(cyclopentylamino)cyclobutanecarboxamide hydrochloride (cpd 154);
N-[5-(2-Chloro-5-cyanophenyl)-1H-indazol-3-yl]-3-(cyclopropylamino)cyclobutanecarboxamide hydrochloride (cpd 155);
N-[5-(2,6-Dichlorophenyl)-1H-indazol-3-yl]tetrahydro-2H-pyran-4-carboxamide (cpd 156);
N-[5-(2,6-Dichlorophenyl)-1H-indazol-3-yl]piperidine-3-carboxamide hydrochloride (cpd 157);
1-Cyclopropyl-N-[5-(2,6-dichlorophenyl)-1H-indazol-3-yl]piperidine-4-carboxamide hydrochloride (cpd 158);
N-[5-(2,6-Dichlorophenyl)-1H-indazol-3-yl]-1-phenylpiperidine-4-carboxamide hydrochloride (cpd 159);
N-[5-(2,6-Dichlorophenyl)-1H-indazol-3-yl]-1-azabicyclo[2.2.2]octane-4-carboxamide (cpd 160);
N-[5-(2-Chloro-6-fluorophenyl)-1H-indazol-3-yl]-1-methylpiperidine-3-carboxamide hydrochloride (cpd 161);
N-[5-(2,6-Dichlorophenyl)-1H-indazol-3-yl]-2-[(2S)-pyrrolidin-2-yl]acetamide hydrochloride (cpd 162);
N-[5-(2,6-Dichlorophenyl)-1H-indazol-3-yl]-2-(piperidin-4-yl)acetamide hydrochloride (cpd 163);
trans-4-(Aminomethyl)-N-[5-(2,6-dichlorophenyl)-1H-indazol-3-yl]cyclohexanecarboxamide hydrochloride (cpd 164);
N-[5-(2-Chloro-6-fluorophenyl)-1H-indazol-3-yl]piperidine-3-carboxamide hydrochloride (cpd 165);
N-[5-(2,6-Dichlorophenyl)-1H-indazol-3-yl]-4-(dimethylamino)cyclohexanecarboxamide hydrochloride (cpd 166);
(3R)-N-[5-(2,6-Dichlorophenyl)-1H-indazol-3-yl]pyrrolidine-3-carboxamide hydrochloride (cpd 167);
(3S)-N-[5-(2,6-Dichlorophenyl)-1H-indazol-3-yl]pyrrolidine-3-carboxamide hydrochloride (cpd 168);
(3R)-N-[5-(2,6-Dichlorophenyl)-1H-indazol-3-yl]piperidine-3-carboxamide hydrochloride (cpd 169);
(3S)-N-[5-(2,6-Dichlorophenyl)-1H-indazol-3-yl]piperidine-3-carboxamide hydrochloride (cpd 170);
N-[5-(2-Chloro-5-cyanophenyl)-1H-indazol-3-yl]piperidine-3-carboxamide hydrochloride (cpd 171);
(3R)-N-[5-(2-Chloro-6-fluorophenyl)-1H-indazol-3-yl]piperidine-3-carboxamide hydrochloride (cpd 172);
(3S)-N-[5-(2-Chloro-6-fluorophenyl)-1H-indazol-3-yl]piperidine-3-carboxamide hydrochloride (cpd 173);
(3R)-N-[5-(2-Chloro-5-cyanophenyl)-1H-indazol-3-yl]piperidine-3-carboxamide hydrochloride (cpd 174);
N-[5-(2,6-Dichlorophenyl)-1H-indazol-3-yl]-b-alaninamide hydrochloride (cpd 175);
(3R)-N-[5-(2-Chloro-5-cyanophenyl)-1H-indazol-3-yl]pyrrolidine-3-carboxamide hydrochloride (cpd 176);
(3R,6S)-N-[5-(2,6-Dichlorophenyl)-1H-indazol-3-yl]-6-methylpiperidine-3-carboxamide trifluoroacetate (cpd 177);
(3R,6R)-N-[5-(2,6-Dichlorophenyl)-1H-indazol-3-yl]-6-methylpiperidine-3-carboxamide trifluoroacetate (cpd 178);
(3R,6R)-N-[5-(2-Chloro-5-cyanophenyl)-1H-indazol-3-yl]-6-methylpiperidine-3-carboxamide trifluoroacetate (cpd 179);
(3R,6S)-N-[5-(2-Chloro-5-cyanophenyl)-1H-indazol-3-yl]-6-(trifluoromethyl)piperidine-3-carboxamide (cpd 180);
(3S)-N-[5-(2-Chloro-5-cyanophenyl)-1H-indazol-3-yl]piperidine-3-carboxamide hydrochloride (cpd 181);
(2R)-N-[5-(2-Chloro-5-cyanophenyl)-1H-indazol-3-yl]morpholine-2-carboxamide hydrochloride (cpd 182);
N-[5-(2-Chloro-5-cyanophenyl)-1H-indazol-3-yl]azepane-3-carboxamide hydrochloride (cpd 183);
N-[5-(2-Chloro-5-cyanophenyl)-1H-indazol-3-yl]-6,6-dimethylpiperidine-3-carboxamide hydrochloride (cpd 184);
N-[5-(2-Chloro-5-cyanophenyl)-1H-indazol-3-yl]-6-azaspiro[4.5]decane-8-carboxamide hydrochloride (cpd 185);
N-[5-(2-Chloro-5-cyanophenyl)-1H-indazol-3-yl]-1-azaspiro [5.5]undecane-3-carboxamide hydrochloride (cpd 186);
N-[5-(2-Chloro-5-cyanophenyl)-1H-indazol-3-yl]-2-azabicyclo[2.2.2]octane-4-carboxamide hydrochloride (cpd 187);
N-[5-(2-Chloro-5-cyanophenyl)-1H-indazol-3-yl]-1-azabicyclo [2.2.2]octane-3-carboxamide hydrochloride (cpd 188);
1-[(2,2-Dimethylpentanoyl)oxy]ethyl (3R)-3-{1[5-(2-chloro-5-cyanophenyl)-1H-indazol-3-yl]carbamoyl}-piperidine-1-carboxylate (cpd 189);
1-[(2-Methylpropanoyl)oxy]ethyl (3R)-3-{[5-(2-chloro-5-cyanophenyl)-1H-indazol-3-yl]carbamoyl}piperidine-1-carboxylate (cpd 190);
(Acetyloxy)methyl (3R)-3-{[5-(2-chloro-5-cyanophenyl)-1H-indazol-3-yl]carbamoyl}piperidine-1-carboxylate (cpd 191);
[(2-Methylpropanoyl)oxy]methyl (3R)-3-{[5-(2-chloro-5-cyanophenyl)-1H-indazol-3-yl]carbamoyl}piperidine-1-carboxylate (cpd 192);
[(2,2-Dimethylpropanoyl)oxy]methyl (3R)-3-{[5-(2-chloro-5-cyanophenyl)-1H-indazol-3-yl]carbamoyl}-piperidine-1-carboxylate (cpd 193);
1-(Acetyloxy)ethyl (3R)-3-{[5-(2-chloro-5-cyanophenyl)-1H-indazol-3-yl]carbamoyl}piperidine-1-carboxylate (cpd 194);
1-[(2,2-Dimethylpropanoyl)oxy]ethyl (3R)-3-{[5-(2-chloro-5-cyanophenyl)-1H-indazol-3-yl]carbamoyl}-piperidine-1-carboxylate (cpd 195);
2-Methyl-1-[(2-methylpropanoyl)oxy]propyl (3R)-3-{[5-(2-chloro-5-cyanophenyl)-1H-indazol-3-yl]carbamoyl}-piperidine-1-carboxylate (cpd 196);
1-[(2,2-Dimethylpropanoyl)oxy]-2-methylpropyl (3R)-3-{[5-(2-chloro-5-cyanophenyl)-1H-indazol-3-yl]carbamoyl}-piperidine-1-carboxylate (cpd 197);
1-{[(1-Methylcyclohexyl)carbonyl]oxy}ethyl (3R)-3-{[5-(2-chloro-5-cyanophenyl)-1H-indazol-3-yl]carbamoyl}-piperidine-1-carboxylate (cpd 198);
1-[(Tetrahydro-2H-pyran-4-ylcarbonyl)oxy]ethyl (3R)-3-{[5-(2-chloro-5-cyanophenyl)-1H-indazol-3-yl]carbamoyl}piperidine-1-carboxylate (cpd 199);
1-[(2,2-Dimethylpropanoyl)oxy]propyl (3R)-3-{[5-(2-chloro-5-cyanophenyl)-1H-indazol-3-yl]carbamoyl}-piperidine-1-carboxylate (cpd 200);
N-[5-(2,6-Dichlorophenyl)-1H-indazol-3-yl]-4-hydroxycyclohexanecarboxamide (cpd 201);
(+)-N-[5-(2-Chloro-5-cyanophenyl)-1H-indazol-3-yl]-6,6-dimethylpiperidine-3-carboxamide (cpd 202);
(−)-N-[5-(2-Chloro-5-cyanophenyl)-1H-indazol-3-yl]-6,6-dimethylpiperidine-3-carboxamide (cpd 203);
(8+)-N-[5-(2-Chloro-5-cyanophenyl)-1H-indazol-3-yl]-6-azaspiro[4.5]decane-8-carboxamide hydrochloride (cpd 204);
(8-)-N-[5-(2-Chloro-5-cyanophenyl)-1H-indazol-3-yl]-6-azaspiro[4.5]decane-8-carboxamide hydrochloride (cpd 205);
(3+)-N-[5-(2-Chloro-5-cyanophenyl)-1H-indazol-3-yl]-1-azaspiro[5.5]undecane-3-carboxamide (cpd 206);
(3-)-N-[5-(2-Chloro-5-cyanophenyl)-1H-indazol-3-yl]-1-azaspiro[5.5]undecane-3-carboxamide (cpd 207);
Butyl 5-(2-chloro-5-cyanophenyl)-3-{1[(3R)-piperidin-3-ylcarbonyl]amino}-1H-indazole-1-carboxylate hydrochloride (cpd 208);
Ethyl 5-(2-chloro-5-cyanophenyl)-3-{[(3R)-piperidin-3-ylcarbonyl]amino}-1H-indazole-1-carboxylate hydrochloride (cpd 209);
Propan-2-yl 5-(2-chloro-5-cyanophenyl)-3-{[(3R)-piperidin-3-ylcarbonyl]amino}-1H-indazole-1-carboxylate hydrochloride (cpd 210);
Hexyl 5-(2-chloro-5-cyanophenyl)-3-{[(3R)-piperidin-3-ylcarbonyl]amino}-1H-indazole-1-carboxylate hydrochloride (cpd 211);
2,2-Dimethylpropyl 5-(2-chloro-5-cyanophenyl)-3-{[(3R)-piperidin-3-ylcarbonyl]amino}-1H-indazole-1-carboxylate hydrochloride (cpd 212);
2-Methylpropyl 5-(2-chloro-5-cyanophenyl)-3-{[(3R)-piperidin-3-ylcarbonyl]amino}-1H-indazole-1-carboxylate hydrochloride (cpd 213);
Pentyl 5-(2-chloro-5-cyanophenyl)-3-{[(3R)-piperidin-3-ylcarbonyl]amino}-1H-indazole-1-carboxylate hydrochloride (cpd 214);
2-Methoxyethyl 5-(2-chloro-5-cyanophenyl)-3-{[(3R)-piperidin-3-ylcarbonyl]amino}-1H-indazole-1-carboxylate hydrochloride (cpd 215);
Cyclobutyl 5-(2-chloro-5-cyanophenyl)-3-{[(3R)-piperidin-3-ylcarbonyl]amino}-1H-indazole-1-carboxylate hydrochloride (cpd 216);
Pentan-3-yl 5-(2-chloro-5-cyanophenyl)-3-{[(3R)-piperidin-3-ylcarbonyl]amino}-1H-indazole-1-carboxylate hydrochloride (cpd 217);
Cyclohexylmethyl 5-(2-chloro-5-cyanophenyl)-3-{[(3R)-piperidin-3-ylcarbonyl]amino}-1H-indazole-1-carboxylate hydrochloride (cpd 218);
Cyclopropylmethyl 5-(2-chloro-5-cyanophenyl)-3-{[(3R)-piperidin-3-ylcarbonyl]amino}-1H-indazole-1-carboxylate formate (cpd 219);
2-(Ethylsulfanyl)ethyl 5-(2-chloro-5-cyanophenyl)-3-{[(3R)-piperidin-3-ylcarbonyl]amino}-1H-indazole-1-carboxylate formate (cpd 220);
3-(3-Methoxypropoxy)propyl 5-(2-chloro-5-cyanophenyl)-3-{1[(3R)-piperidin-3-ylcarbonyl]amino}-1H-indazole-1-carboxylate formate (cpd 221);
4-Methylpentyl 5-(2-chloro-5-cyanophenyl)-3-{[(3R)-piperidin-3-ylcarbonyl]amino}-1H-indazole-1-carboxylate formate (cpd 222);
Cyclopentyl 5-(2-chloro-5-cyanophenyl)-3-{[(3R)-piperidin-3-ylcarbonyl]amino}-1H-indazole-1-carboxylate formate (cpd 223);
2,2-Dimethylpropyl 5-(2-chloro-5-cyanophenyl)-3-({[(3+)-6,6-dimethylpiperidin-3-yl]carbonyl}amino)-1H-indazole-1-carboxylate hydrochloride (cpd 224);
Hexyl 5-(2-chloro-5-cyanophenyl)-3-({[(3R)-6,6-dimethylpiperidin-3-yl]carbonyl}amino)-1H-indazole-1-carboxylate hydrochloride (cpd 225);
Propan-2-yl 5-(2-chloro-5-cyanophenyl)-3-({[(3R)-6,6-dimethylpiperidin-3-yl]carbonyl}amino)-1H-indazole-1-carboxylate hydrochloride (cpd 226);
(3R)-N-[5-(2-Fluoroethoxy)-1H-indazol-3-yl]piperidine-3-carboxamide hydrochloride (cpd 227);
(3R)-N-[5-(2-Methoxyethoxy)-1H-indazol-3-yl]piperidine-3-carboxamide hydrochloride (cpd 228);
(3R)-N-(5-Ethoxy-1H-indazol-3-yl)piperidine-3-carboxamide hydrochloride (cpd 229);
(3R)-N-[5-(Propan-2-yloxy)-1H-indazol-3-yl]piperidine-3-carboxamide hydrochloride (cpd 230); and
(3R)-N-(5-Butoxy-1H-indazol-3-yl)piperidine-3-carboxamide hydrochloride (cpd 231).

5. A process for the preparation of a compound of formula (I) or a pharmaceutical acceptable salt thereof, as defined in claim 1, said process comprises:
st. 1) mixing the compound of formula (III):
Step 1a) reacting a compound of formula (V): 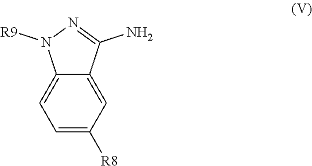
wherein R8 is bromine or 4,4,5,5-tetramethyl-[1,3,2]dioxaborolane and R9 is a protecting group, such as 1,1′,1″-methanetriyltribenzene, tert-butyloxycarbonyl, with a compound of formula (VI): 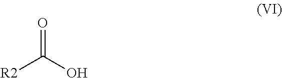
wherein R2 is as defied in claim 1;
Step 1b′) mixing the obtained intermediate of formula (VII): 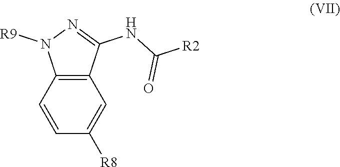
wherein R2 and R9 are defined as above in step 1a and R8 is bromine, with a compound of formula (VIII) or (IX): 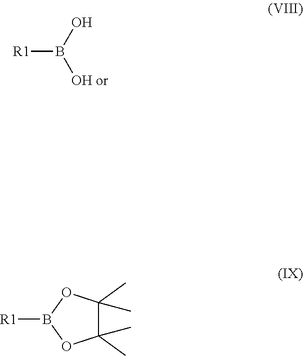
wherein R1 is an optionally substituted straight or branched (C1-C6) alkyl, (C3-C7) cycloalkyl, heterocyclyl, aryl and 5 or 6-membered heteroaryl;
or
Step 1b″) reacting the intermediate of formula (VII) wherein R8 is 4,4,5,5-tetramethyl-[1,3,2]dioxaborolane and R2 and R9 are defined above in step 1a with a compound of formula (X):
R1-X (X)
wherein R1 is defined as above in step 1b′ and X is halogen;
Step 1c) reacting the obtained intermediate (XI): 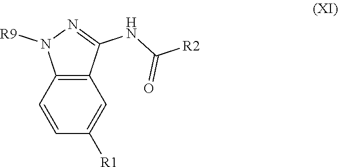
wherein R1, R2 and R9 are defined as above in step 1b′ and 1b″, with the appropriate deprotecting agent to obtain a compound of formula (I): 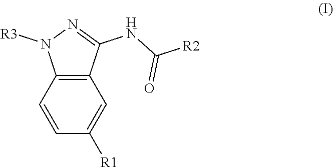
wherein R 3 is hydrogen, R1 is as defined in claim 1 and R2 is defined as above;
or
Step 1d) reacting a compound of formula (V): 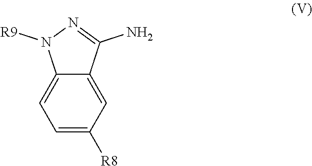
wherein R8 is bromine and R9 is as defined above in step 1a, with a compound of formula (VIII) or (IX): 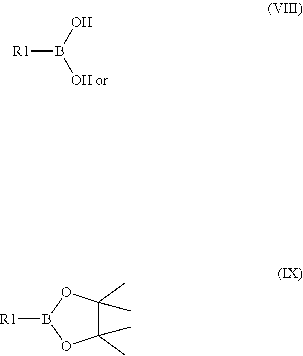
wherein R1 is defined as above in step 1b′;
Step 1e) mixing the resulted intermediate (XII): 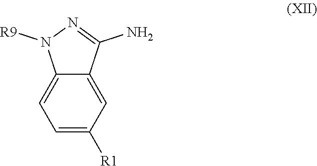
wherein R1 and R9 are as defined above in step 1c, with a compound of formula (VI): 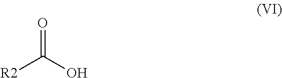
wherein R2 is defined as above in step 1a;
Step 1f) reacting the obtained intermediate (XIII): 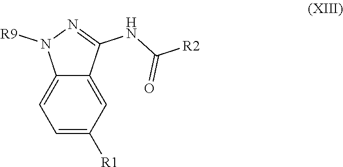
wherein R1, R2 and R9 are defined as above, with the appropriate deprotecting agent thus to obtain a compound of formula (I): 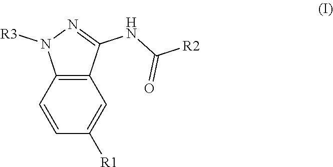
wherein R3 is hydrogen and R1 and R2 are defined as above in step 1c;
Alternatively, a first compound of formula (VII) or (XII) can be converted into a second compound of formula (VII) or (XII), respectively, by operating according conversions reported below:
conv. A) converting a compound of formula (VIIa): 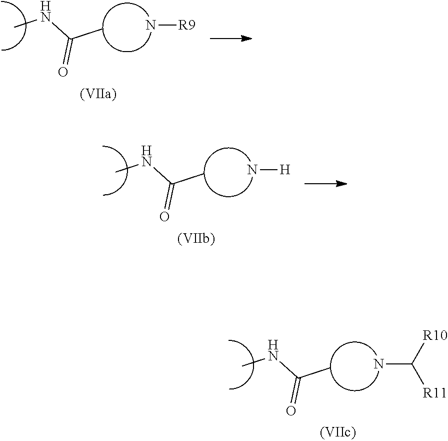
wherein R2 is an optionally substituted heterocyclyl and R9 is defined as above in step 1a, into a compound of formula (VIIb), by first removing the protecting group then, reacting the resulting derivative with a compound of formula R10R11CO (XIV), wherein R10 and R11 are hydrogen, an optionally substituted group selected from straight or branched (C1-C6) alkyl, (C3-C7) cycloalkyl or R10 and R11 taken together may form an optionally substituted (C3-C7) cycloalkyl and heterocyclyl group, under reductive amination conditions in the presence of a suitable reducing agents;
conv. B) converting a compound of formula (VIId): 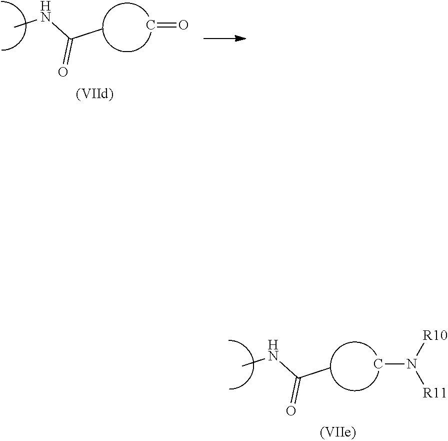
wherein R2 is a (C4-C6) cycloalkyloxo, into a compound of formula (VIIe), by reacting with a compound of formula R10R11NH (XV) wherein R10 and R 11 are defined as above in conv. A, under reductive amination conditions in the presence of a suitable reducing agents;
conv. C) converting a compound of formula (XIIIa): 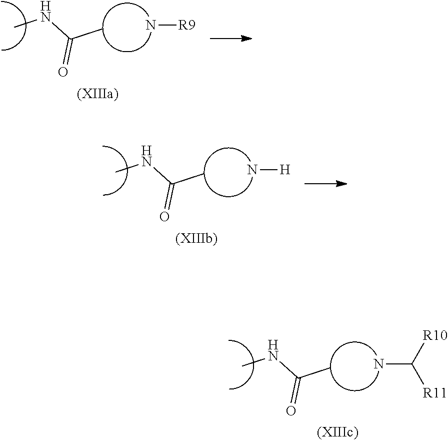
wherein R2 is an optionally substituted heterocycly and R9 is defined as above in step 1a, into a compound of formula (XIIIb), by first removing the protecting group then, reacting the resulting derivative with a compound of formula R10R11CO (XIV), wherein R10 and R11 are defined as above in conv. A, under reductive amination conditions in the presence of a suitable reducing agents;
conv. D) converting a compound of formula (XIIId): 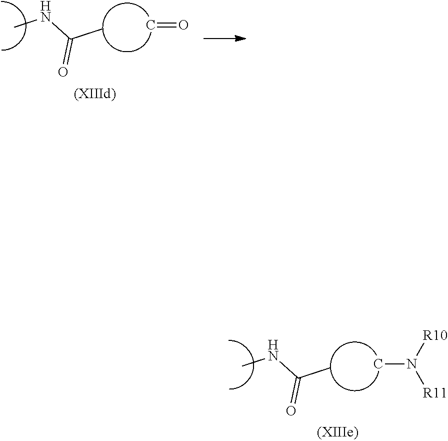
wherein R2 is a (C4-C6) cycloalkyloxo, into a compound of formula (XIIIe), by reacting with a compound of formula R10R11NH (XV) wherein R10 and R11 are defined as above in conv.A, under reductive amination conditions in the presence of a suitable reducing agents;
conv. E) converting a compound of formula (XIIIf): 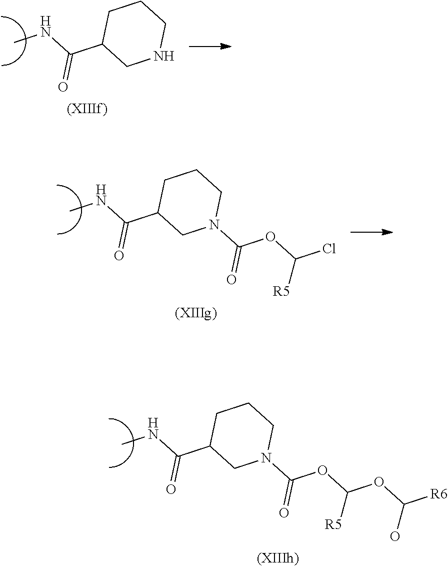
first into a compound of formula (XIIIg), reacting with a compound of formula R5CHClOCOCl (XVI) wherein R5 is as defined in claim 1, then into a compound of formula (XIIIh), wherein R6 is as defined in claim 1, reacting under basic condition with a compound of formula R6COOH (XVII);
or
a first compound of formula (I) can be conveniently converted into a second compound of formula (I) by operating according to conversions reported below:
conv. 1) converting a compound of formula (I): 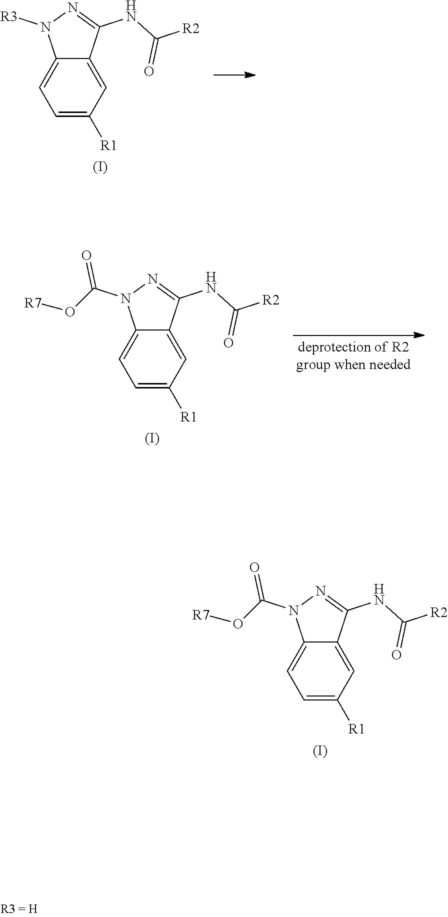
wherein R1 is defined as above in step 1b′ and R2 is an optionally substituted straight or branched (C2-C6) alkyl, (C3-C7) cycloalkyl, heterocyclyl and 5 to 6-membered heterocyclylmethyl wherein the heterocyclyl is selected from the group consisting of pirrolydinyl, and piperidinyl, into a compound of formula (I), by reacting with a compound of formula R7OCOCl (XIX) wherein R7 is defined as in claim 1, under basic condition;
or
compounds of formula (I) wherein R1 is a group of formula (II) wherein R4 is an optionally substituted straight or branched (C2-C6) alkyl, (C3-C7) cycloalkyl or heterocyclyl, can be prepared accordingly, the process comprises the following steps:
Step 2a) reacting a compound of formula (XX): 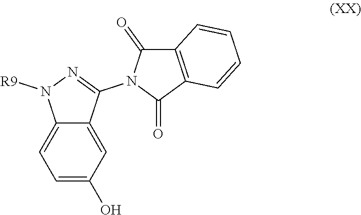
wherein R9 is defined as above in step 1a, with a compound of formula (XXI):
R4-Y (XXI)
wherein R4 is as defined in claim 1;
Step 2b) reacting the obtained intermediate (XXII): 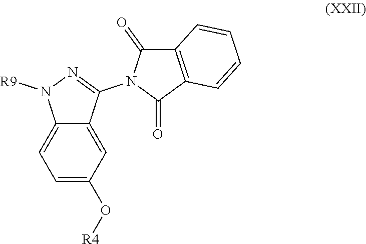
with the suitable deprotecting agent;
Step 2c) mixing the obtained intermediate (XXIII): 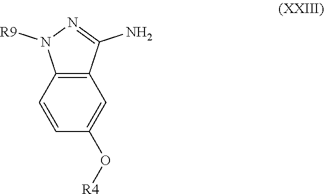
with a compound of formula (VI): 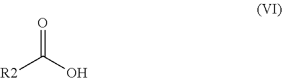
wherein R2 is defined as above in step 1a;
Step 2d) reacting the obtained intermediate (XXIV): 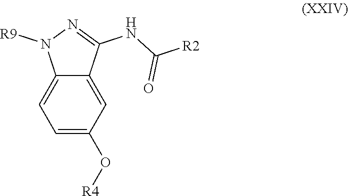
with the suitable deprotecting agent to obtain a compound of formula (I): 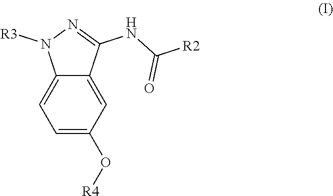
wherein R3 is hydrogen and R2 and R4 are as defined in claim 1.

6. (canceled)

7. (canceled)

8. (canceled)

9. (canceled)

10. (canceled)

11. A method of treating a disease caused by and/or associated with dysregulated CDK11 kinase activity which comprises administering to a mammal, preferably a human, in need thereof, an effective amount of a compound of formula (I) as defined in claim 1.

12. The method, according to claim 11, wherein the disease is selected from the group consisting of cancer, cell proliferative disordes and immune-related disorders.

13. The method, according to claim 12, wherein the disease is cancer.

14. The method according to claim 11, wherein said cancer is selected from the group consisting of: carcinomas, such as carcinomas, such as carcinomas, such as bladder, breast, kidney, liver, colon, lung, including small cell lung cancer, esophagus, gall-bladder, ovary, pancreas, stomach, cervix, prostate, and skin, including squamous cell carcinoma; hematopoietic tumors of lymphoid lineage including leukemia, acute lymphocitic leukemia, acute lymphoblastic leukemia, chronic lymphocytic leukemia, B-cell lymphoma, angioimmunoblastic T-cell lymphoma, Hodgkin's lymphoma, non-Hodgkin's lymphoma, hairy cell lymphoma and Burkitt's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including glioma, glioblastoma, glioblastoma multiforme, astrocytoma, oligodendroglioma, paraglioma, neuroblastoma, and schwannomas; and other tumors, including melanoma, seminoma, teratocarcinoma, osteosarcoma, xeroderma pigmentosum, keratoxanthoma, thyroid cancers, such as papillary thyroid carcinoma and medullary thyroid carcinoma, Kaposi's sarcoma, chondrosarcoma, and cholangiocarcinoma.

15. A pharmaceutical composition comprising a compound of formula (I) or a pharmaceutically acceptable salt thereof, as defined in claim 1, in association with a pharmaceutically acceptable excipient, carrier or diluent.

16. A pharmaceutical composition according to claim 15 further comprising one or more chemotherapeutic agents.

17. A product or kit comprising a compound of formula (I) or a pharmaceutically acceptable salt thereof, as defined in claim 1, and one or more chemotherapeutic agents, as a combined preparation for simultaneous, separate or sequential use in anticancer therapy.

18. (canceled)

19. The method according to claim 10 in combination with radiation therapy or with a chemotherapy regimen.
